(19) United States
(12) Patent Application Publication
(10) Pub. No.: US 2023/0245716 A1
(43) Pub. Date: Aug. 3, 2023

(54) Systems and Methods for Stable and Heritable Alteration by Precision Editing (SHAPE)

(71) Applicant: The General Hospital Corporation, Boston, MA (US)

(72) Inventors: J. Keith Joung, Winchester, MA (US); Luca Pinello, Jamaica Plain, MA (US); Jonathan Hsu, Cambridge, MA (US); Julian Grunewald, Charlestown, MA (US); Y. Esther Tak, Charlestown, MA (US); Md Nafiz Hamid, Quincy, MA (US)

(21) Appl. No.: 17/928,105

(22) PCT Filed: May 28, 2021
(86) PCT No.: PCT/US2021/034996

§ 371 Date: Nov. 28, 2022

Related U.S. Application Data
(60) Provisional application No. 63/032,486, filed on May 29, 2020.

(51) Int. Cl. G16B 20/30; G16B 30/10; C12N 9/22; C12N 15/90

(52) U.S. Cl. CPC G16B 20/30; G16B 30/10; C12N 9/22; C12N 15/907; C12N 2310/20

(57) ABSTRACT

Described herein are systems, methods, and compositions for the precise editing of DNA sequence(s) at specific loci to alter expression of target gene products at the pre-transcriptional or post-transcriptional level in a durable fashion, termed Stable and Heritable Alteration by Precision Editing (SHAPE). The SHAPE platform utilizes genetic modifiers (e.g., nucleases, (CRISPR guided) transposases, recombinases, base editors, and prime editors) to install specific sequence motifs at target sequences through precision genome engineering.

### CLAIM OF PRIORITY

This application is a U.S. National Phase Application under 35 U.S.C. § 371 of International Patent Application No. PCT/US2021/034996, filed on May 28, 2021, which claims the benefit of U.S. Provisional patent application Ser. No. 63/032,486, filed on May 29, 2020. The entire contents of the foregoing are hereby incorporated by reference.

### FEDERALLY SPONSORED RESEARCH OR DEVELOPMENT

This invention was made with Government support under Grant Nos. GM118158, CA211707, CA204954, and HG010717 awarded by the National Institutes of Health. The Government has certain rights in the invention.

### SEQUENCE LISTING

This application contains a Sequence Listing that has been submitted electronically as an ASCII text file named ‘29539 0527US1 Sequence Listing’. The ASCII text file, created on Nov. 21, 2022, is 891 kilobytes in size. The material in the ASCII text file is hereby incorporated by reference in its entirety.

### TECHNICAL FIELD

Described herein are systems, methods, and compositions for the precise editing of DNA sequence(s) at specific loci to alter expression of target gene products at the pre-transcriptional or post-transcriptional level in a durable fashion, termed Stable and Heritable Alteration by Precision Editing (SHAPE). The SHAPE platform utilizes genetic modifiers (e.g., nucleases, (CRISPR guided) transposases, recombinases, base editors, and prime editors) to install specific sequence motifs at target sequences through precision genome engineering.

### BACKGROUND

Precisely controlling gene expression in biosystems has important applications in biotechnology and therapeutic settings^{5 6 6, 7}. Pre-transcriptional strategies for gene regulation include the use of artificial transcription factors (ATFs) where a programmable DNA-binding domain (e.g., zinc fingers, transcription activator-like effectors, CRISPR-Cas) is coupled with an effector domain (e.g., VP64, p65, KRAB) to alter gene transcription⁶, whereas post-transcriptional strategies include targeted protein degradation (TPD) and RNA interference (RNAi). These gene regulation strategies are transient in nature and require re-dosing or constitutive expression of the exogenous biomolecules to have durable and sustained effect(s). There remains a need for durable gene regulation strategies that don't require constitutive presence of exogenous biomolecules.

### SUMMARY

Described herein are genome engineering strategies for the precise installation of sequence motifs at specific loci to alter expression of target gene products at the pre-transcriptional or post-transcriptional level, termed Stable and Heritable Alteration by Precision Editing (SHAPE). The SHAPE platform includes the identification of 1) functional sequence motifs with regulatory potential, 2) target regions for sequence modification to take place, and 3) genetic modifiers to use to achieve the precise edit of interest to ultimately induce targeted gene expression change(s) for a cell type or cell types of interest (Table 1 and 2).

Thus, provided herein are methods for identifying a method for altering expression of target genes in selected cell types.

Thus, provided herein are methods for identifying a genetic modifier to alter expression of a target gene in a selected cell type. The methods include: providing, optionally from a database, one or more candidate regulatory motif sequences with regulatory potential (as described herein, e.g., binding sites for transcription factors or other factors that affect gene expression and are expressed in the cell, e.g., endogenous factors) in the selected cell type; selecting a sequence of a putative regulatory region of the target gene, preferably wherein the putative regulatory region is in a promoter, enhancer, insulator, untranslated region (UTR), or intron, optionally in a non-coding region of the target gene; comparing the sequence of the putative regulatory region to the candidate regulatory motif sequences, identifying a candidate regulatory motif sequence that has either little to no identity at all (e.g., for the insertion strategy) as a potential insertion site or that has at least 50% identity and at least one mismatch (i.e., not 100% identity, for the substitution strategy) to a (corresponding) portion of the sequence of the regulatory sequence as a potential substitution site;

determining sequence alterations needed to make the putative regulatory region match (e.g., to include or have 100% identity with) the candidate regulatory motif sequence; and

identifying one or more genetic modifiers capable of making the sequence alterations needed to make the putative regulatory region match the candidate regulatory motif sequence. In some embodiments, identifying a genetic modifier comprises using a computer or an algorithm that compares the putative regulatory region and candidate regulatory motif sequences from a database and identifies candidate regulatory motif sequences that differ from the putative regulatory region by at least one nucleotide and up to 100% as a potential insertion site, or that differ from the putative regulatory region by at least one nucleotide and up to a selected amount, optionally at least 50% identity, as a potential substitution site, determines sequence alterations needed to make the putative regulatory region match the candidate regulatory motif sequence, compares the sequence alterations to a database of modifications that could be made by a set of genetic modifiers, and identifying one or more genetic modifiers that can alter the putative regulatory region to match the candidate regulatory motif sequence, to thereby introduce a functional regulatory motif.

In some embodiments, the candidate regulatory sequence motif has regulatory potential to affect target gene expression at the pre-transcriptional or post-transcriptional level.

In some embodiments, the candidate regulatory sequence motif is a transcription factor binding sequence that can recruit endogenous transcription factors within a cell type or cell types of interest (e.g., cell type-specific factors), where the sequence motif may or may not exist in the genome of the selected cell type.

In some embodiments, the candidate regulatory sequence motif alters spacing of endogenous transcription factor binding sites in the putative regulatory region.

In some embodiments, the candidate regulatory sequence motif is a response element that is activated by a receptor-ligand complex through binding of an exogenously delivered small molecule, hormone, or drug for inducible target gene activation.

In some embodiments, the candidate regulatory sequence motif either stabilizes or de-stabilizes target gene transcripts, where the candidate regulatory sequence motif may or may not exist in the genome of the selected cell type.

In some embodiments, the candidate regulatory sequence motif is a hybridization target for endogenous non-coding RNAs (e.g., miRNAs, siRNAs, lncRNAs), where the sequence motif may or may not exist in the genome of interest.

In some embodiments, the candidate regulatory sequence motif modifies the translation initiation and/or elongation efficiency for target gene transcripts (e.g., Kozak sequence, optimal codon structure), and wherein the candidate regulatory sequence motif may or may not exist in the genome of the selected cell type.

In some embodiments, the putative regulatory region has the potential to modify expression of the target gene at the pre-transcriptional or post-transcriptional level.

In some embodiments, the putative regulatory region is a non-coding DNA sequence within 1 Mb or more of a target gene of interest, or spatially-proximal as determined by chromosome conformation capture assays.

In some embodiments, the putative regulatory region is a promoter of a target gene of interest, e.g., a proximal regions e.g., 1000 bp upstream and 500 bp downstream of the transcription start site (TSS).

In some embodiments, the putative regulatory region comprises putative enhancer elements of a target gene of interest as defined by histone marks associated and/or chromatin accessibility features associated with functional enhancer elements (e.g., H3K4me1, H3K27ac); putative insulator elements of the target gene of interest as defined by histone marks associated and/or chromatin accessibility features associated with functional insulator elements; and/or putative silencer elements of the target genes of interest as defined by histone marks associated and/or chromatin accessibility features associated with functional silencer elements.

In some embodiments, the putative regulatory region comprises untranslated regions (UTRs) of the target gene transcripts.

In some embodiments, the putative regulatory regions comprise an intronic region of the target gene transcripts.

In some embodiments, the putative regulatory regions comprises a coding sequence of target gene transcripts.

In some embodiments, the identified genetic modifier can introduce a specific sequence motif or modification at the target genomic region.

In some embodiments, the genetic modifier comprises a CRISPR-Cas domain, a zinc-finger DNA binding domain, or a transcription activator-like (TAL) effector domain.

In some embodiments, the CRISPR-Cas domain is used with a gRNA, wherein the gRNA comprises a sequence complementary to a sequence of the target cis-regulatory element of interest.

In some embodiments, the genetic modifier is a programmable nuclease (e.g., zinc finger nucleases, transcription activator-like effector nucleases, Cas9, CasX, Cas12), a base editor (e.g., ABE, CBE), or a prime editor (e.g., SpCas9H840A-MMLV-RT).

In some embodiments, the CRISPR-Cas prime editor further comprises a prime editing gRNA (pegRNA) and nicking sgRNA (ngRNA) wherein the pegRNA and ngRNA comprise a sequence complementary to a sequence of the target cis-regulatory element of interest.

Also provided herein are methods for altering expression of a target gene in a selected cell type. The methods include providing, optionally from a database, one or more candidate regulatory motif sequences with regulatory potential in the selected cell type; selecting a sequence of a putative regulatory region of the target gene, preferably wherein the putative regulatory region is in a promoter, enhancer, insulator, untranslated region (UTR), or intron, optionally in a non-coding region of the target gene; comparing the sequence of the putative regulatory region to the candidate regulatory motif sequences, identifying a candidate regulatory motif sequence that has either no identity at all as a potential insertion site or that has at least 50% identity and at least one mismatch to a portion of the sequence of the regulatory sequence as a potential substitution site; determining sequence alterations needed to make the putative regulatory region match the candidate regulatory motif sequence; and identifying one or more genetic modifiers capable of making the sequence alterations needed to make the putative regulatory region match the candidate regulatory motif sequence, and contacting the cell with the one or more genetic modifiers under conditions and for a time sufficient for the one or more genetic modifiers to make the putative regulatory region match the candidate regulatory motif sequence.

In some embodiments, identifying a genetic modifier comprises using a computer or an algorithm that compares the putative regulatory region and candidate regulatory motif sequences from a database and identifies candidate regulatory motif sequences that differ from the putative regulatory region by at least one nucleotide and up to 100% as a potential insertion site, or that differ from the putative regulatory region by at least one nucleotide and up to a selected amount, optionally at least 50% identity, as a potential substitution site, determines sequence alterations needed to make the putative regulatory region match the candidate regulatory motif sequence, compares the sequence alterations to a database of modifications that could be made by a set of genetic modifiers, and identifying one or more genetic modifiers that can alter the putative regulatory region to match the candidate regulatory motif sequence, to thereby introduce a functional regulatory motif.

Also provided herein are methods for altering expression of a target gene in a selected cell, the method comprising contacting the selected cell with a genetic modifier identified using a method described herein, under conditions sufficient to increase the target gene expression in the cell.

Additionally, provided herein are methods for heterotopic activation of a target gene expression in a selected cell, the method comprising contacting the cell with a genetic modifier identified using a method described herein, under conditions sufficient to increase the target gene expression in the cell.

In some embodiments, the candidate regulatory sequence motif is introduced into the putative regulatory region as a single motif or a repetitive sequence with multiple copies of the single motif, optionally with linker sequences therebetween.

In some embodiments, the genetic modifier introduces multiplex edits (e.g., installation of multiple transcription factor binding sites) in order to induce more robust modification of a single target gene expression. For example, a single type of modifier such as a prime editor or CRISPR Cas domain containing protein can be used, wherein that modifier is guided to multiple locations in the genome via multiple guide RNAs to enable multiplex edits.

In some embodiments, the genetic modifier introduces multiplex edits (e.g., installation of multiple transcription factor binding sites) in order to perform multi-gene expression control.

In some embodiments, the cell is a eukaryotic cell, e.g., a mammalian cell, e.g., a human cell.

Also provided herein are methods for treating or reducing risk of a condition or a disease in a subject, wherein the condition or the disease is caused, at least in part, by insufficient expression of the target gene, the method comprising administering to the subject an effective amount of a genetic modifier identified using a method described herein, under conditions sufficient to increase the target gene expression in the cell, thereby treating or reducing risk of the condition or the disease in the subject.

In some embodiments, the condition or the disease is caused, at least in part, by insufficient expression of the target gene on an allele.

In some embodiments, the condition or the disease is related to haploinsufficiency.

In some embodiments, the condition or the disease is caused, at least in part, by a dominant-negative gene.

In some embodiments, the condition or the disease is caused, at least in part, by insufficient expression of a target gene that is under the control of an enhancer, wherein the enhancer controls the expression of a plurality of genes.

In some embodiments, the method causes an increase in the expression of the target gene in the cell or in the cell of the subject by at least 1.1 fold as measured by mRNA expression.

Also provided herein are method for treating or reducing risk of a condition or a disease in a subject, wherein the condition or the disease is caused, at least in part, by overexpression of the target gene, the method comprising administering to the subject an effective amount of a genetic modifier identified using a method as described herein, under conditions sufficient to increase the target gene expression in the cell, thereby treating or reducing risk of the condition or the disease in the subject.

In some embodiments, the method causes a decrease in the expression of the target gene in the cell or in the cell of the subject by at least 1.1 fold.

In some embodiments, the subject is a mammal, e.g., a human.

In some embodiments, the present methods include:
Identifying one or more regulatory motif sequences with regulatory potential in the selected cell type or tissue;
Identifying a sequence of a putative regulatory region of the target gene;
Comparing the sequence of the regulatory region to the regulatory motif sequences, and identifying a candidate motif sequence that has:
(A) For a substitution-based strategy: at least 5% identity and at least one mismatch (i.e., not 100% identity) to a portion of the sequence of the regulatory sequence;
   identifying a genetic modifier (e.g. base or prime editors) capable of altering the regulatory region to match (have 50-100% identity with) the candidate motif sequence, sufficient to create a binding site.
(B) For an insertion-based strategy: no homology between the endogenous target sequence and the intended created motif is needed. The nucleotides/bases that include the regulatory sequence or motif will be inserted into a regulatory region of any gene of interest. Following the identification of the regulatory motif and target sequence for genome engineering, a genetic modifier (e.g. prime editors, nuclease for homology directed repair (HDR) strategy, targeted transposases or recombinases) is identified that is capable of altering the regulatory region to match (have 100% identity with) the candidate motif sequence.
(C) For an indel-based strategy: DSBs via genetic modifiers (e.g. SpCas9) and the resulting insertion and deletion (indel) mutations are often predictable (e.g. +1 insertions, MMEJ patterns) to a certain degree and can be utilized to introduce new binding sites (50-100% identity with candidate motifs) for the recruitment of transcription factors (activators or repressors). Furthermore, indel mutations can also introduce different spacing between endogenous transcription factor binding sites, leading to an alternative way to modulate target gene expression.

The methods can include using an algorithm that compares the target regulatory regions and regulatory motif sequences identified above and identifies candidate regulatory motif sequences that differ from the target gene regulatory region by up to 100% for the insertion strategy and less than a selected amount, e.g., by 95% for the insertion strategy, and compares the candidate regulatory motifs to the possible modifications that would be made by a set of genetic modifiers (e.g., to predict the modification(s) made by each of a set of genetic modifiers, to identify one or more genetic modifiers that can be used to modify the target regulatory region to introduce a functional regulatory motif.

The methods can include CRISPR-guided multiplex gene editing with guide RNAs targeting a nuclease or base/prime editor at two, three, or more, e.g., up to 25, endogenous target sites to introduce multiple regulatory sequences in a given cell type or tissue to modify gene regulation at one or multiple genes in parallel (Campa et al, Nature Methods 2019, Vol 16, pp 887-893). In the referenced paper Cas12a is used for gene editing. This enzyme has been shown to work efficiently in the context of 2^{nd} generation CRISPR tools as well, such as e.g. base editors (Richter et al, Nat Biotechnol (2020). doi.org/10.1038/s41587-020-0453-z).

Also provided herein are methods for identifying a genetic modifier to alter expression of a target gene in a selected cell type, the methods comprising:

Identifying one or more regulatory motif sequences with regulatory potential in the selected cell type;
Identifying a sequence of a putative regulatory region of the target gene, preferably wherein the regulatory region is in a promoter, enhancer, insulator, UTR, or intron, optionally in a non-coding region of the target gene;
Comparing the sequence of the regulatory region to the regulatory motif sequences, and identifying a candidate motif sequence that has either no homology at all in case of the insertion strategy or that has at least 50% identity and at least one mismatch (i.e., not 100% identity) to a portion of the sequence of the regulatory sequence in case of the substitution strategy; and Identifying a genetic modifier capable of altering the regulatory region to match (have 100% identity with) the candidate motif sequence, preferably wherein the genetic modifier is a zinc finger nuclease, CRISPR-Cas9 nuclease, base editor, or prime editor
optionally comprising using an algorithm that compares the target regulatory regions and regulatory motif sequences and identifies candidate regulatory motif sequences that differ from the target gene regulatory region by up to 1-100% for the insertion strategy and less than a selected amount, e.g., by 95% for the substitution strategy and compares the candidate regulatory motifs to the possible modifications that would be made by a set of genetic modifiers, to identify one or more genetic modifiers that can be used to modify the target regulatory region to introduce a functional regulatory motif.

In some embodiments, the identified or discovered sequence motif or modification has regulatory potential to affect target gene expression at the pre-transcriptional or post-transcriptional level.

In some embodiments, the identified or discovered sequence motif is a transcription factor binding sequence that can recruit endogenous transcription factors within a cell type or cell types of interest (cell type-specific), where the sequence motif that may or may not exist in the genome of interest.

In some embodiments, the identified or discovered sequence modification alters the spacing of endogenous transcription factor binding sites in the genome.

In some embodiments, the sequence motif is a response element that is activated by a receptor-ligand complex through binding of an exogenously delivered small molecule, hormone, or drug for inducible target gene activation.

In some embodiments, the identified or discovered sequence motif or modification either stabilizes or de-stabilizes target gene transcripts, where the sequence motif that may or may not exist in the genome of interest.

In some embodiments, the identified or discovered sequence motif is a hybridization target for endogenous non-coding RNAs (e.g., miRNAs, siRNAs, lncRNAs), where the sequence motif that may or may not exist in the genome of interest.

In some embodiments, the identified or discovered sequence motif modifies the translation initiation and/or elongation efficiency for target gene transcripts (e.g., Kozak sequence, optimal codon structure), where the sequence motif that may or may not exist in the genome of interest.

In some embodiments, the identified or discovered sequence motif or modification is determined by a combination of gene expression (e.g., RNA-seq), chromatin accessibility (e.g., ATAC-seq, DNase-seq), DNA-protein interaction (e.g., ChIP-seq), and/or primary DNA sequence data from a single cell type or set of cell types of interest.

In some embodiments, the identification or discovery of sequence motifs can be performed by integrative analysis of genomics data across different cell types, e.g., using a computational strategy wherein regions of interest are uncovered based on their cell type specific activity for a particular class of functional regions and on genomic data e.g., chromatin marks (e.g., H3k27ac, H3k27me3), chromatin accessibility (e.g., DNase-seq or ATAC-seq) or DNA methylation; based on the recovered regions and a list of known TF motifs, regions are searched for enriched patterns and their significance evaluated; integrating gene expression data a short list of candidate TF are provided to account for their endogenous expression across the different cell types and their expected potency based on genes that are downstream of the regions uncovered in the second step; and a ranked list of TF sequences is generated based on this integrative approach for each cell type.

In some embodiments, the discovery of the sequence motifs is done by de novo motif discovery analysis within a single cell type or set of cell types of interest.

In some embodiments, the discovery of sequence motifs is done by analyzing cis-regulatory DNA sequence composition of top-expressing genes (e.g., top 1%, 5%, 20%, 50%) ranked by normalized expression values (e.g., RPKM, FPKM, TPM, fold-change) in a single cell type or set of cell types of interest.

In some embodiments, the discovery of the sequence motifs is done by analyzing cis-regulatory DNA sequence composition of bottom-expressing genes (e.g., bottom 1%, 5%, 20%, 50%) ranked by normalized expression values (e.g., RPKM, FPKM, TPM, fold-change) in a single cell type or set of cell types of interest.

In some embodiments, the discovery of the sequence motifs is done through frequency-based methods including the construction of position-weight matrices for a single cell type or set of cell types of interest.

In some embodiments, the discovery of the sequence motifs is done through neural network architectures to identify sequence motifs that may or may not exist in a single cell type or set of cell types of interest.

In some embodiments, the discovery of the sequence motifs is done through generation of synthetic DNA sequences using language models—a generative deep learning technique—where the sequence motifs may or may not exist in a single cell type or set of cell types of interest.

In some embodiments, the discovery of the sequence motifs is done through generation of synthetic DNA sequences using deep variational autoencoders—a generative deep learning model—where the sequence motifs may or may not exist in a single cell type or set of cell types of interest.

In some embodiments, the discovery of the sequence motifs is done through generation of synthetic DNA sequences using Generative Adversarial Networks (GANs)—a generative deep learning model—where the sequence motifs may or may not exist in a single cell type or set of cell types of interest.

In some embodiments, the discovery of the sequence motifs is done through the identification of transcription factor binding sequence motifs with dependencies with other sequence motifs (e.g., pairwise, triwise interactions).

In some embodiments, the discovery of the sequence motifs is done through the identification of transcription factor binding sequence motifs that recruit additional transcriptional machinery through protein-protein interactions.

In some embodiments, the identified sequence motif is from an online database for transcription factor binding motifs (e.g., JASPAR, HOCOMOCO).

In some embodiments, the sequence motif is introduced as a single motif or a repetitive sequence with multiple copies of the single motif that may or may not have linker sequences interspaced.

In some embodiments, the sequence motif is introduced as a combination of different sequence motifs with predicted additive or synergistic effects on target gene expression at the pre-transcriptional and/or post-transcriptional level, where the multiple sequence motifs may or may not have linker sequences interspaced.

In some embodiments, the target genomic region to introduce the sequence motif or modification is able to or has the potential to modify target gene expression at the pre-transcriptional or post-transcriptional level.

In some embodiments, the target genomic regions are non-coding DNA sequences within 1 Mb or more of the target gene(s) of interest, or spatially-proximal as determined by chromosome conformation capture assays.

In some embodiments, the target genomic regions are promoters of the target gene(s) of interest, defined as proximal regions e.g., 1000 bp upstream and 500 bp downstream of the transcription start site (TSS).

In some embodiments, the target genomic regions are putative enhancer elements of the target gene(s) of interest defined by histone marks associated and/or chromatin accessibility features associated with functional enhancer elements (e.g., H3K4me1, H3K27ac).

In some embodiments, the target genomic regions are putative insulator elements of the target gene(s) of interest defined by histone marks associated and/or chromatin accessibility features associated with functional insulator elements.

In some embodiments, the target genomic regions are putative silencer elements of the target gene(s) of interest defined by histone marks associated and/or chromatin accessibility features associated with functional silencer elements.

In some embodiments, the target genomic regions are untranslated regions (UTRs) of target gene transcripts.

In some embodiments, the target genomic regions are intronic regions of target gene transcripts.

In some embodiments, the target genomic regions are coding sequences of target gene transcripts.

In some embodiments, the genetic modifier to use is able to introduce the specific sequence motif or modification at the target genomic region with sufficient efficiency and precision.

In some embodiments, the genetic modifier comprises a CRISPR-Cas domain, a zinc-finger DNA binding domain, or a transcription activator-like (TAL) effector domain.

In some embodiments, the genetic modifier is a programmable nuclease (e.g., zinc finger nucleases, transcription activator-like effector nucleases, Cas9, CasX, Cas12).

In some embodiments, the CRISPR-Cas domain further comprises a gRNA wherein the gRNA comprises a sequence complementary to a sequence of the target cis-regulatory element of interest.

In some embodiments, the genetic modifier is a base editor (e.g., ABE, CBE).

In some embodiments, the genetic modifier is a prime editor (e.g., SpCas9H840A-MMLV-RT).

In some embodiments, the CRISPR-Cas prime editor further comprises a prime editing gRNA (pegRNA) and nicking sgRNA (ngRNA) wherein the pegRNA and ngRNA comprise a sequence complementary to a sequence of the target cis-regulatory element of interest.

In some embodiments, the genetic modifier introduces multiplex edits (e.g., installation of multiple transcription factor binding sites) in order to induce more robust modification of a single target gene expression.

In some embodiments, the genetic modifier introduces multiplex edits (e.g., installation of multiple transcription factor binding sites) in order to perform multi-gene expression control.

In some embodiments, the use of unbiased saturation mutagenesis screening to empirically determine genetic editing modalities (e.g., programmable nucleases, base editors, prime editors) and target sites (e.g., promoter, enhancers, UTRs) to modify target gene(s) expression.

The present methods can be used for increasing a target gene expression in a cell, and for heterotopic activation of a target gene expression in a cell, by contacting the cell with a genetic modifier identified using a method described herein.

In some embodiments, the cell is a eukaryotic cell.

In some embodiments, the cell is a mammalian cell.

In some embodiments, the cell is a human cell.

Also provided are methods for treating or preventing a condition or a disease in a subject, the method comprising administering to the subject an effective amount of a genetic modifier identified by a method described herein, e.g., in a pharmaceutical composition, thereby treating or preventing the condition or the disease.

In some embodiments, the condition or the disease is caused, at least in part, by insufficient expression of the target gene.

In some embodiments, the condition or the disease is caused, at least in part, by insufficient expression of the target gene on an allele.

In some embodiments, the condition or the disease is related to haploinsufficiency.

In some embodiments, the condition or the disease is caused, at least in part, by a dominant-negative gene.

In some embodiments, the administration of the pharmaceutical composition increases expression of the target gene, thereby treating the condition or the disease.

In some embodiments, the condition or the disease is caused, at least in part, by insufficient expression of a target gene that is under the control of an enhancer, wherein the enhancer controls the expression of a plurality of genes.

In some embodiments, the method causes increase in the expression of the target gene in the cell or in the cell of the subject by at least 1.1 fold, 2 fold, at least 3 fold, at least 4 fold, at least 5 fold, at least 6 fold, at least 7 fold, at least 8 fold, at least 9 fold, at least 10 fold, at least 15 fold, at least 20 fold, at least 25 fold, at least 30 fold, at least 35 fold, at least 40 fold, at least 45 fold, at least 50 fold, at least 60 fold, at least 70 fold, at least 80 fold, at least 90 fold, at least 100 fold, at least 150 fold, at least 200 fold, at least 300 fold, at least 350 fold, at least 400 fold, at least 450 fold, at least 500 fold, at least 600 fold, at least 700 fold, at least 800 fold, at least 900 fold, at least 1000 fold, at least 1100 fold, at least 1200 fold, at least 1300 fold, at least 1400 fold, at least 1500 fold, at least 1600 fold, at least 1700 fold, at least 1800 fold, at least 1900 fold, at least 2000 fold, at least 2500 fold, or at least 3000 fold, as measured by mRNA expression.

In some embodiments, the method causes a decrease in the expression of the target gene in the cell or in the cell of the subject by at least 1.1 fold, 2 fold, at least 3 fold, at least 4 fold, at least 5 fold, at least 6 fold, at least 7 fold, at least 8 fold, at least 9 fold, at least 10 fold, at least 15 fold, at least 20 fold, at least 25 fold, at least 30 fold, at least 35 fold, at least 40 fold, at least 45 fold, at least 50 fold, at least 60 fold, at least 70 fold, at least 80 fold, at least 90 fold, at least 100 fold, at least 150 fold, at least 200 fold, at least 300 fold, at least 350 fold, at least 400 fold, at least 450 fold, at least 500 fold, at least 600 fold, at least 700 fold, at least 800 fold, at least 900 fold, at least 1000 fold, at least 1100 fold, at least 1200 fold, at least 1300 fold, at least 1400 fold, at least 1500 fold, at least 1600 fold, at least 1700 fold, at least 1800 fold, at least 1900 fold, at least 2000 fold, at least 2500 fold, or at least 3000 fold, as measured by mRNA expression.

In some embodiments, the methods include providing a cell of the selected cell type and contacting the cell with the genetic modifier to alter the regulatory region to match the candidate motif sequence.

In some embodiments, the methods alter expression of gene products at the pre-transcriptional (e.g., recruitment of endogenous transcription factors, transcription activation or repression) or post-transcriptional level (e.g., sequence motifs that modify transcript stability, sequence motifs that modify translation initiation and/or elongation efficiency), in the context of a single cell type or set of cell types of interest.

In some embodiments, identifying one or more candidate motif sequences with regulatory potential in the selected cell type comprises referring to a database comprising a plurality of regulatory sequence motifs, e.g., endogenous regulatory sequences that are present in the selected cell type (e.g., in the species of the cell) but not in the target gene, or exogenous regulatory sequences (e.g., not present in the cell, from a different species, or artificial regulatory sequences) that bind to a factor present in the cell (e.g., a transcription factor, target sequence for endogenous non-coding RNAs, a sequence motif in the untranslated regions (UTR) of a transcript that increases or decreases the stability and/or affects the transcription of the RNA molecules, and/or sequence motif that modifies the translation initiation or elongation efficiency of transcripts, in the selected cell type.

In some embodiments, the candidate sequence motif is a transcription factor binding sequence, e.g., a binding sequence that is endogenous (present in the genome, but not in the gene, of the cell of interest), or exogenous (e.g., not present in the cell of interest, e.g., an artificial TF binding site or a TF binding site from another cell type or species that binds an endogenous TF that is expressed in the cell) of interest (Table 3A).

In some embodiments, the candidate sequence motif is a range for spacing between endogenous transcription factor binding sites that modifies gene expression.

In some embodiments, the candidate sequence motif is a known sequence motif in the untranslated regions (UTR) of transcripts that increases or decreases the stability and/or affects the transcription of these RNA molecules in cells, either endogenous or exogenous.

In some embodiments, the candidate sequence motif is a known target sequence for endogenous non-coding RNAs (e.g., miRNAs, siRNAs, lncRNAs) that affect the target transcript stability.

In some embodiments, the candidate sequence motif is a sequence motif that is exogenous (not present in the genome of interest), but that recruits endogenous non-coding RNAs (e.g., miRNAs, siRNAs, lncRNAs) that affect the target transcript stability (Table G, H, I, and J).

In some embodiments, the candidate sequence motif is an (endogenous or exogenous) sequence motif that modifies the translation initiation (e.g., Kozak sequence) or elongation (e.g., codon optimization) efficiency of transcripts.

In some embodiments, the candidate sequence motif encodes a 2A self-cleaving peptide (e.g., T2A, P2A, E2A, F2A).

In some embodiments, the candidate sequence motif encodes an intein sequence.

In one aspect, the present application includes identifying target genomic regions to modify that, upon the introduction of specific sequence motifs within this genomic region, may alter expression of a target gene or set of target genes at the pre-transcriptional or post-transcriptional level, in the context of a cell type or set of cell types of interest.

In some embodiments, the target genomic regions are non-coding DNA sequences within 1 Mb or more of the target gene(s) of interest.

In some embodiments, the target genomic regions are promoters of the target gene(s) of interest, defined as proximal regions e.g., 1000 bp upstream and 500 bp downstream of the transcription start site (TSS).

In some embodiments, the target genomic regions are putative enhancer elements of the target gene(s) of interest defined by histone marks and/or chromatin accessibility features associated with functional enhancer elements (e.g., H3K4me1, H3K27ac)¹⁷.

In some embodiments, the target genomic regions are putative insulator elements of the target gene(s) of interest defined by histone marks and/or chromatin accessibility features associated with functional insulator elements^{17,18}.

In some embodiments, the target genomic regions are putative silencer elements of the target gene(s) of interest defined by histone marks and/or chromatin accessibility features associated with functional silencer elements¹⁹.

In some embodiments, the target genomic regions are untranslated regions (UTRs) of target gene transcripts.

In some embodiments, the target genomic regions are intronic regions of target gene transcripts.

In some embodiments, the target genomic regions are coding sequences of target gene transcripts.

In some embodiments, the endogenous regulatory region of a gene (e.g., the promoter) is targeted to modify or enhance downstream transcription of translation machinery. This can be achieved e.g., by installing or modifying a TATA box (also known as Goldberg-Hogness box) in archae or eukaryotes or a Pribnow box in prokaryotes or by installing enhanced Kozak ((gcc)gccRccAUGG (SEQ ID NO: 478)) in eukaryotes, Shine-Dalgarno (AGGAGGU) in prokaryotes, start codon (AUG and CUG in mammalian cells, AUA and AUU in mitochondria, GUG and UUG in E. coli) or stop codon (UGA, UAG, UAA) sequences²⁰.

In some embodiments binding sites of non-coding RNAs (ncRNAs), such as microRNAs (miRNAs) or long non-coding RNAs (lncRNAs) are installed or modified to alter the binding of said ncRNAs to DNA or RNA with the result of altered gene expression, and/or RNA abundance, and/or protein expression.

The methods can include producing a list of candidate sequence modifications that can be made to add a candidate motif to the target sequence.

The present methods also include identifying genetic modifiers that can introduce specific candidate sequence motifs into target genomic regions with high predicted precision and efficiency that may alter expression of a target gene or set of genes at the pre-transcriptional or post-transcriptional level, in the context of a cell type or set of cell types of interest. Genetic modifiers can include a programmable nuclease (e.g., zinc finger nucleases, transcription activator-like effector nucleases, and CRISPR-Cas systems, e.g., Cas9, CasX, Cas12); base editors (e.g. ABEs, CBEs, and CGBEs); and prime editors, inter alia (Table 2).

In some embodiments, algorithms can be used to identify a genetic modifier, e.g., based on comparing the desired sequence modifications to be made to the changes that could be made by a number of genetic modifiers.

In some embodiments, the sequence motif is to be introduced as a single motif or a repetitive sequence with multiple copies of the single motif that may or may not have linker sequences interspaced.

In some embodiments, the sequence motif is to be introduced as a combination of different sequence motifs with predicted additive or synergistic effects on target gene expression at the pre-transcriptional and/or post-transcriptional level, where the multiple sequence motifs may or may not have linker sequences interspaced.

In some embodiments, the methods can include predicting a sequence modification that would be caused by one genetic modifiers, e.g., using an algorithm, e.g., a computer-based method.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims

### DESCRIPTION OF DRAWINGS

FIG. 1. Illustration of the MYOD1 locus and experimental designs for prime editing. Illustration of the human MYOD1 gene, open chromatin features at MYOD1 locus from ATAC-seq from HEK293T cell line, and pegRNAs and nicking sgRNAs locations. Three pegRNAs (Table B) were designed upstream of MYOD1, and two nicking sgRNAs (Table C) were designed downstream of each pegRNA.

FIG. 2. Quantification of the desired transcription factor motif insertion event by prime editing across multiple motif sequences. Only perfect insertion events by prime editing were included in this analysis. A total of 3 pegRNAs (A, B, and C) and 10 transcription factor motifs (Table D) were tested. The length of insertions were either single copies of the transcription factor binding motif (1×) or two copies of the single motifs without an intervening linker sequence (2×). Each pegRNA was paired with 3 different nicking sgRNAs (1 upstream, 2 downstream) and the editing efficiencies are grouped into each bar.

FIG. 3. MYOD1 gene expression changes with ELF motif insertion. RNA expression levels of the endogenous MYOD1 gene in HEK293T in the presence of PE3, pegRNA that has ELF motif, and nicking sgRNA. For motif #, 1× and 2× indicate that pegRNA has single or double ELF motifs, respectively. Three pegRNAs (A, B, C) and two nicking sgRNAs for each pegRNA were tested. MYOD1 transcript levels were measured by RT-qPCR, normalized to HPRT1 levels, and values shown are normalized relative to a control sample (labelled Empty) in which pegRNA cassette was expressed.

FIG. 4. MYOD1 gene expression changes with NFY motif insertion. RNA expression levels of the endogenous MYOD1 gene in HEK293T in the presence of PE3, pegRNA that has NFY motif, and nicking sgRNA. For motif #, 1× and 2× indicate that pegRNA has single or double NFY motifs, respectively. Three pegRNAs (A, B, C) and two nicking sgRNAs for each pegRNA were tested. MYOD1 transcript levels were measured by RT-qPCR, normalized to HPRT1 levels, and values shown are normalized relative to a control sample (labelled Empty) in which pegRNA cassette was expressed.

FIG. 5. MYOD1 gene expression changes with GATA motif insertion. RNA expression levels of the endogenous MYOD1 gene in HEK293T in the presence of PE3, pegRNA that has GATA1 motif, nicking sgRNA, and exogenous expression of GATA1. For motif #, 1×, 2×, and 3× indicate that pegRNA has single or double or triple GATA1 motifs, respectively. Three pegRNAs (A, B, C) and two nicking sgRNAs for each pegRNA were tested. MYOD1 transcript levels were measured by RT-qPCR, normalized to HPRT1 levels, and values shown are normalized relative to a control sample (labelled Empty) in which pegRNA cassette was expressed.

FIG. 6. MYOD1 gene expression changes with SP motif insertion. RNA expression levels of the endogenous MYOD1 gene in HEK293T in the presence of PE3, pegRNA that has SP1 motif, and nicking sgRNA. For motif #, 1× indicates that pegRNA has a single SP1 motif. Three pegRNAs (A, B, C) and two nicking sgRNAs for each pegRNA were tested. MYOD1 transcript levels were measured by RT-qPCR, normalized to HPRT1 levels, and values shown are normalized relative to a control sample (labelled Empty) in which pegRNA cassette was expressed.

FIG. 7. MYOD1 gene expression changes with EWS-FLI1 motif insertion. RNA expression levels of the endogenous MYOD1 gene in HEK293T in the presence of PE3, pegRNA that has EWS-FLI1 motif, nicking sgRNA, and exogenous EWS-FLI1. For motif #, 3× and 6× indicate that pegRNA has triple or sextuple of GGAA motifs. Three pegRNAs (A, B, C) and two nicking sgRNAs for each pegRNA were tested. MYOD1 transcript levels were measured by RT-qPCR, normalized to HPRT1 levels, and values shown are normalized relative to a control sample (labelled Empty) in which pegRNA cassette was expressed.

FIGS. 8A-B. Genome-wide analysis of possible CCA>TTG base edits in the human genome in gene promoters that could create transcription factor binding sites. A) Bar plots showing computationally determined number of genes (y-axis) that could be targeted by SPACE to install 1-10 (or more) transcription factor (TF) binding sites (x-axis) in proximity of the transcription start site of the coding genes in the human genome. Sites were filtered to contain a preferential SPACE editing window (C3-C4-A5 with 1 being the most PAM-distal position) and a canonical NGG-PAM. B) Bar plots showing computationally determined number of genes (y-axis) that could be targeted by SPACE to install 1-5 (or more) transcription factor binding sites (x-axis) in proximity of the transcription start site of coding genes in the human genome for 4 transcription factors. Sites were filtered to contain a preferential SPACE editing window (C3-C4-A5) and a canonical NGG-PAM (Table A).

FIG. 9. Additional genome-wide analysis to detect possible CCA>TTG base edits in the human genome in gene promoters that could create transcription factor binding sites by using the dual-deaminase base editor SPACE (BE-SHAPE). Bar plots showing computationally determined number of genes (y-axis) that could be targeted by SPACE to install 1-5 (or more) transcription factor binding sites (x-axis) in proximity of the transcription start site of coding genes in the human genome for additional 6 transcription factors. Sites were filtered to contain a preferential SPACE editing window (C3-C4-A5) and a canonical NGG-PAM.

FIGS. 10A-C. Base editing with the dual-deaminase base editor SPACE establishes synthetic transcription factor binding sites at target promoters by installing highly complex combinatorial substitutions. A) Allele frequency table from next-generation sequencing (NGS) data shows one allele with SPACE-induced dual base edits (black rectangle; 3.32% frequency) that introduces a binding site for the transcription factor CEBPA in the promoter of the gene RCC1. Four more alleles with edits that might enhance TF binding but are less congruent to the canonical TF binding site are depicted in the rectangles with black dashed lines (frequencies of 30.18%, 2.47%, 2.09%, and 2.04% respectively). B) Allele frequency table from next-generation sequencing (NGS) data shows one allele with SPACE-induced dual base edits (black rectangle; 10.78% frequency) that introduces a binding site for the transcription factor NFIX in the promoter of the gene RARA. One more allele with edits that might enhance TF binding but is less congruent to the canonical TF binding site is depicted in the rectangle with black dashed lines (frequency of 1.96%). C) Allele frequency table from next-generation sequencing (NGS) data shows one allele with SPACE-induced dual base edits (black rectangle; 10.15% frequency) that introduces a binding site for the transcription factor PBX1 in the promoter of the gene IVD. One more allele with edits that might enhance TF binding but is less congruent to the canonical TF binding site is depicted in the rectangle with black dashed lines (frequency of 12.5%).

FIGS. 11A-C. Editing with the base editor SPACE results in gene expression changes of target genes (BE-SHAPE). A) Allele frequency table from next-generation sequencing (NGS) data shows two alleles with SPACE-induced dual base edits (black rectangles; 13.1% and 3% frequency, respectively) that introduce a binding site for the transcription factor NFIX in the promoter of the gene COMT. B) Allele frequency table from next-generation sequencing (NGS) data shows one allele with SPACE-induced dual base edits (black rectangles; 9.8% frequency) that introduce a binding site for the transcription factor CEBPA in the promoter of the gene SAE1.Three more alleles with edits that might enhance TF binding but are less congruent to the canonical TF binding site are depicted in the rectangles with black dashed lines (frequencies of 18.4%, 13.4%, and 3.0% respectively). C) Fold-change of expression (y-axis) observed in SPACE-edited HEK293T cells for five tested genes (x-axis). For each gene, nCas9 or SPACE (left and right bar) was targeted to the promoter region (˜500 bp to transcription start site, TSS) in two independent replicates, measured with 3 separate RT-qPCR measurements each. Dots represent separate RT-qPCR measurements. Error bars represent standard deviation (SD).

FIG. 12. Overview of the SHAPE workflow. The SHAPE strategy is aimed at introducing genetic edits to modify the gene expression of a target gene or genes at either the pre- or post-transcriptional level. A) Given a target gene or genes of interest (e.g. haploinsufficiency disease), the SHAPE pipeline identifies B) cell type-specific regulatory motifs (e.g. TF motifs, miRNA targets), C) genetic editors (e.g. nucleases, base editors, prime editors) to install these identified regulatory motifs with high precision, and D) regions to perform the precise genetic editing (e.g. promoters, enhancers, insulators, UTRs) to affect target gene(s) expression. Following the identification of these elements, E) precise genetic editing is performed to F) induce target gene(s) expression changes.

FIG. 13A-B. Stable MYOD1 expression by ELF motif insertion via prime editing but not with dCas9-VPR recruitment in HEK293T. A) Stable ELF(2×) motif insertion at the promoter of MYOD1 over 30 days post-transfection with prime editor (PE) and GFP construct. B) A comparison of PE and dCas9-VPR in stable activation of MYOD1 expression over 30 days post-transfection. U: unsorted, S: sorted

FIGS. 14A-B. Stable IL2RA expression by ELF motif insertion via prime editing but not with dCas9-VPR recruitment in HEK239T. A) Stable ELF(2×) motif insertion at the promoter of IL2RA over 30 days post-transfection with prime editor (PE) and GFP construct. B) A comparison of PE and dCas9-VPR in stable activation of IL2RA expression over 30 days post-transfection. U: unsorted, S: sorted

FIGS. 15A-B. Stable EpCAM expression by ELF motif insertion via prime editing but not with dCas9-VPR recruitment in HEK239T. A) Stable ELF(2×) motif insertion at the promoter of EpCAM over 30 days post-transfection with prime editor (PE) and GFP construct. B) A comparison of PE and dCas9-VPR in stable activation of EpCAM expression over 30 days post-transfection. U: unsorted, S: sorted

FIGS. 16A-B. Stable MYOD1 expression by ELF motif insertion via prime editing but not with dCas9-VPR recruitment in K562. A) Stable ELF(2×) motif insertion at the promoter of MYOD1 over 30 days post-transfection with prime editor (PE) and GFP construct. B) Prime editing led stable activation of MYOD1 expression over 30 days post-transfection. U: unsorted, S: sorted

FIGS. 17A-B. Stable HBB expression by ELF motif insertion via prime editing but not with dCas9-VPR recruitment in K562. A) Stable ELF(2×) motif insertion at the promoter of HBB over 30 days post-transfection with prime editor (PE) and GFP construct. B) Prime editing led stable activation of HBB expression over 30 days post-transfection. U: unsorted, S: sorted.

FIGS. 18A-B. Stable IL2RA expression by ELF motif insertion via prime editing but not with dCas9-VPR recruitment in K562. A) Stable ELF(2×) motif insertion at the promoter of IL2RA over 30 days post-transfection with prime editor (PE) and GFP construct. B) Prime editing led stable activation of IL2RA expression over 30 days post-transfection. U: unsorted, S: sorted

FIGS. 19A-C. Stable HER2 and protein expression by ELF motif insertion via prime editing but not with dCas9-VPR recruitment in K562. A) Stable ELF(2×) motif insertion at the promoter of HER2 over 30 days post-transfection with PE. B) Prime editing led stable activation of HER2 expression over 30 days post-transfection. C) A comparison of PE and dCas9-VPR in stable activation of HER2 protein expression. U: unsorted, S: sorted

FIGS. 20A-B. Stable IL2RA mRNA and protein expression by ELF motif insertion via prime editing but not with dCas9-VPR recruitment in K562. A) Stable ELF(2×) motif insertion at the promoter of IL2RA over 30 days post-transfection with PE. B) A comparison of PE and dCas9-VPR in stable activation of IL2RA protein expression. U: unsorted, S: sorted

FIGS. 21A-C. Stable EpCAM mRNA and protein expression by ELF motif insertion via prime editing but not with dCas9-VPR recruitment in HEK239T. A) Stable ELF(2×) motif insertion at the promoter of EpCAM over 30 days post-transfection with PE. B) A comparison of PE and dCas9-VPR in stable activation of EpCAM expression. C) A comparison of PE and dCas9-VPR in stable activation of EpCAM protein expression. U: unsorted, S: sorted

FIG. 22. Pooled screening of transcription factor cluster motif insertions via prime editing for activation and repression of IL2RA expression in HEK239T. A library of 100 archetype sequences derived from hierarchal-clustered transcription factor motifs (Table E) and relevant controls were inserted via prime editing into the IL2RA promoter to assess each motif's regulatory potential for either activation or repression of IL2RA expression. A total of 5 negative sequences, generated via a random sequence generator, were included in the screen to serve as negative controls (dark grey). A positive control of ELF(2×) motif was also included (black). Following prime editing of the insertion library, cells were sorted based on IL2RA protein expression at the cell surface into negative and positive populations. Next-generation DNA sequencing was performed to assess the proportion of each insertion sequence within the two populations and read counts were used to calculate enrichment (log 2 fold-change) for the insertion sequences.

FIG. 23. Pooled screening of single-base mutagenized ELF motif insertion via prime editing for tuneable IL2RA expression in HEK293T. A library of single-base mutagenized ELF(2×) motif sequences (Table F) were inserted via prime editing into the IL2RA promoter to assess single-base genetic variants of the ELF(2×) motif for the tuning of IL2RA expression. A total of 5 negative sequences, generated via a random sequence generator, were included in the screen to serve as negative controls (dark grey). A positive control of ELF(2×) motif was also included (black). Following prime editing of the insertion library, cells were sorted based on IL2RA protein expression at the cell surface into negative and positive populations. Next-generation DNA sequencing was performed to assess the proportion of each insertion sequence within the two populations and read counts were used to calculate enrichment (log 2 fold-change) for the inserted sequences.

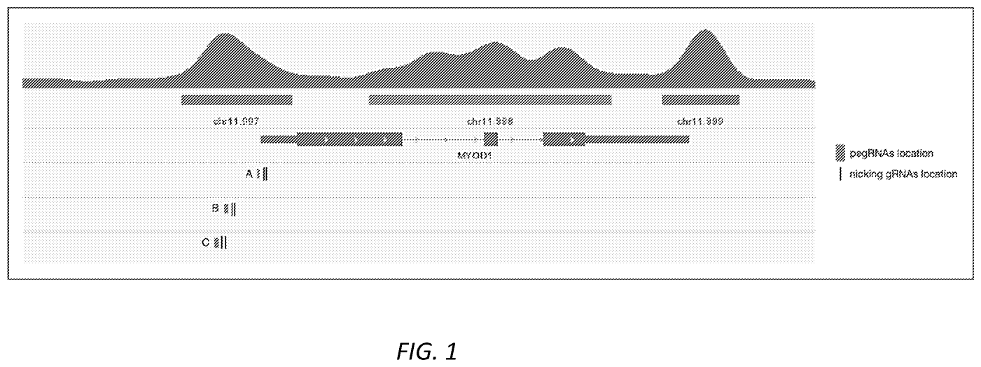

### DETAILED DESCRIPTION

Precisely controlling gene expression in biosystems has important applications in biotechnology and therapeutic settings. Pre-transcriptional strategies for gene regulation include the use of artificial transcription factors (ATFs), whereas post-transcriptional strategies include targeted protein degradation (TPD) and RNA interference (RNAi). These gene regulation strategies are transient in nature and require re-dosing or constitutive expression of the exogenous biomolecules to have durable effect(s).

The regulation of endogenous gene expression is not a simple process, with dynamic transcriptional changes occurring across biological processes such as differentiation and disease. The DNA primary sequence plays a role in this gene regulation control, but how it encodes robust or dynamic gene expression modules is not clear. For example, many sequence motifs exist in the genome, however only a subset of these sequence motifs are bound by transcription factors. Previously, it was not clear that the introduction of a sequence motif into a new context can lead to the modification of gene expression.

Installing a single TF motif at an inactive promoter could lead to increased gene expression. It is well known that a lot of TFs work in concert to bring co-factors and RNA polymerases for gene expression, which might require complex TF motifs at the target promoters or enhancers.

Here we describe a strategy to introduce sequence motifs into non-endogenous contexts, where these sequence motifs recruit endogenous transcription factors that lead to the modification of gene expression. As shown herein, a single TF motif insertion modulates gene expression at a target gene. A single ELF motif which is ˜12 bp inserted at MYOD1 promoter increased ˜20 fold of MYOD1 gene expression in HEK293T. These results suggest we can modulate gene expression by introducing a single TF motif for endogenously active TF in cell types of interest and titrate gene expressions by inserting multiple copies of this single TF motif.

Described herein are strategies for the targeted modification (e.g., activation or repression) of gene expression in a stable and heritable manner, which we call Stable and Heritable Alteration by Precision Editing (SHAPE). The SHAPE methodology is based on the targeted and precise introduction of sequence motifs at regions in the genome that enable changes in target gene expression at either the pre-transcriptional or post-transcriptional level.

The SHAPE platform utilizes genetic modifiers (e.g., nucleases, (CRISPR guided) transposases, recombinases, base editors, and prime editors) to install specific sequence motifs at target sequences through precision genome engineering. Insertion and deletion (indel) mutations from programmable nucleases (e.g., zinc finger nucleases, transcription activator-like effector nucleases, Cas9, CasX, Cas12), base edits from cytosine and/or adenine base editors (e.g., CBEs, ABEs), and all types of prime edits from prime editors (e.g., SpCas9H840A-MMLV-RTPE3) can be utilized to introduce specific sequence motifs at target non-coding DNA sequences (e.g., promoters, enhancers, insulators) or coding (e.g., exons) to influence the gene expression or untranslated regions (UTRs) of target genes. The newly-introduced edits can take the form of different regulatory elements, including: transcription factor binding sites (TFBSs) that recruit endogenous transcription factors to modify target gene transcription, sequence modifications that alter the spacing of endogenous TFBSs, sequence elements that modify the stability (e.g., stabilize, de-stabilize) of target gene transcripts, or sequence compositions that modify target gene translation initiation and/or elongation. Following the introduction of these genetic edits, target gene expression can be modified in a stable and heritable fashion. If the newly introduced motif recruits endogenous transcriptional machinery (e.g., transcription factors), stabilizes the RNA of target genes, or drives expression of novel transcriptional products, the expression of target genes can be altered in a stable and heritable fashion.

SHAPE Platform

The SHAPE platform provides a framework, e.g., a computational (computer-implemented) framework, for the identification or generation of 1) functional DNA sequence motifs with regulatory potential, 2) target regions for sequence modification, and 3) genetic modifiers to use to ultimately achieve the precise installation of sequence motifs that induce targeted gene expression change(s) at the pre-transcriptional or post-transcriptional level. Overall, the SHAPE platform enables genetically-encoded gene regulation through the precise installation of sequence motifs within endogenous DNA sequence, offering a differentiated strategy for stable and heritable transcriptional modulation at the pre-transcriptional or post-transcriptional level with the transient expression of genetic modifiers.

An overview of different exemplary strategies of SHAPE for heritable targeted gene activation is outlined in Table 1, where gene activation strategies are mapped to the possible edit types. These edit types are then mapped to potential genetic modifiers that have the ability to introduce the specific edit in Table 2.

TABLE 1
Overview of SHAPE strategies

| | | | Gene expression | |
|---|---|---|---|---|
| | Level of | | modification | |
| System | regulation | Target elements | strategy | Edit type(s) |
| Mammalian, insect, | Pre- | Non-coding sequences | De novo TFBS | Substitution |
| bacterial, | transcriptional | (e.g., promoters, | creation | Insertion |
| plant, yeast | | enhancers, insulators, | | Deletion |
| | | silencer/repressors, | | Combination |
| | | introns) | TFBS sequence | Substitution |
| | | | modification(s) | |
| | | | TFBS spacing | Insertion |
| | | | modification(s) | Deletion |
| | | | | Combination |
| | Post- | 5′ and/or 3′ | Transcript | Substitution |
| | transcriptional | untranslated regions | stabilization/ | Insertion |
| | | (UTRs) | destabilization | Deletion |
| | | | | Combination |
| | | Coding sequences | Translation | Substitution |
| | | (e.g., translation start | initiation/elongation | Insertion |
| | | sites, codon | | Deletion |
| | | structures) | | Combination |

TABLE 2
Edit type breakdown with relevant genetic modifiers

| Edit type(s) | Specific edit type | Genetic modifier type |
|---|---|---|
| Substitution | Transition | Base editors, prime editors |
| | Transversion | Base editors, prime editors |
| | Mixed | Prime editors |
| Insertion | 1-2 bp | Nucleases, prime editors |
| | >2 bp | Prime editors, nucleases + ssODNs |
| | | (HDR), nucleases + dsODNs |
| | | (NHEJ) |
| Deletion | MMEJ-driven | Nucleases, prime editors |
| | Non-MMEJ | Dual nucleases, prime editors |
| Combination | Substitution, | Prime editors |
| | insertion, deletion | |
| Inversion | Inversion | Dual nucleases |

Generation of novel synthetic DNA sequence motifs that can recruit endogenous transcription factors can be done using a generative technique known as language modeling. A language model is typically a probability distribution over sequence of words that can occur in a natural language e.g., English or French. They are typically trained to predict the next word in a sentence. After training is done, the model can be used to generate novel sentences that are semantically meaningful. This type of modeling can also be used to generate de novo DNA sequences. The model typically uses a neural network where the layers consist of Long Short Term Memory (LSTM) units. The neural network can be trained on a large corpora of DNA sequences, and then fine-tuned on known DNA sequence motifs in a conditional manner to generate novel synthetic DNA motifs that are functional in terms of recruiting transcription factors.

The modification of endogenous transcription factor sequence composition by genetic editing is a viable strategy for gene activation. While transcription factors typically bind a core DNA sequence, there can be minor differences in the totality of sequences transcription factors bind across a genome. In addition to a range of different DNA sequences a transcription factor is capable of binding, the transcription factor may also exhibit different binding strength for each DNA sequence variant.′ The identification of the optimal binding sequence of transcription factors and downstream regulatory potential of this binding event can be determined through integration of chromatin accessibility (e.g., ATAC-seq, DNase-seq), protein-DNA interaction (e.g., ChIP-seq) and gene expression (e.g., RNA-seq) data. Following the identification of top DNA sequence binding motifs for all transcription factors, the identification of sub-optimal endogenous transcription factor binding motifs in cis-regulatory regions of a target gene can be performed. The utilization of genetic modifiers to introduce substitution edits (e.g., base editors, prime editors) can introduce more optimal transcription factor binding sites to promote heritable target gene activation.

Identification of Candidate Regulatory Sequence Motifs

The identification or generation of regulatory sequence motifs to be introduced into the genome can be determined through integrative analysis of by gene expression, chromatin accessibility, and/or DNA-protein interactions data, and de novo motif discovery, or generative neural network frameworks within a single cell type or set of cell types of interest. These sequence motifs can take the form of binding motifs for the recruitment of endogenous transcription factors, sequence motifs that promote the stabilization of RNA molecules, or sequence motifs that enable the expression of non-coding RNA for target gene repression.

Exemplary candidate regulatory sequence motifs can include one or more of a transcription factor binding sequence that has been determined experimentally in-vitro (e.g., SELEX)⁸, experimentally in-vivo (e.g., ChIP-seq, ChIP-qPCR)⁹, or computationally^{10 11} based on experimental data; a sequence motif that is not present in the genome of interest, but has been predicted to facilitate transcription factor binding; a known range for spacing between endogenous transcription factor binding sites that has been shown to modify gene expression; a predicted range for spacing between endogenous transcription factor binding sites that has been predicted to modify gene expression; a known sequence motif in the untranslated regions (UTR) of transcripts that increases or decreases the stability and/or affects the transcription of these RNA molecules in cells¹²; a sequence motif that is not present in the genome of interest, but has been predicted to modify the stability and/or affects the transcription of RNA molecules when placed in 5′ and/or 3′ untranslated regions (UTRs); a known target sequence for endogenous non-coding RNAs (e.g., miRNAs, siRNAs, lncRNAs) that affects the target transcript stability¹³; a sequence motif that is not present in the genome of interest, but has been predicted to recruit endogenous non-coding RNAs (e.g., miRNAs, siRNAs, lncRNAs) that affects the target transcript stability; a known sequence motif that modifies the translation initiation (e.g., Kozak sequence) or elongation (e.g., codon optimization) efficiency of transcripts¹⁴; a sequence motif that is not present in the genome of interest, but has been predicted to modify the translation initiation (e.g., Kozak sequence) or elongation (e.g., codon optimization) efficiency of transcripts; a sequence encoding a 2A self-cleaving peptide (e.g., T2A, P2A, E2A, F2A); and/or a sequence encoding an intein sequence.

A number of methods are known in the art for identifying candidate sequence motifs.

In some embodiments, the identification of candidate DNA sequence motifs is performed by integrative analysis of genomics data across different cell types and with a computational strategy we have recently proposed called Haystack¹⁵. First, regions of interest are uncovered based on their cell type specific activity for a particular class of functional regions and on genomic data e.g., chromatin marks (e.g., H3k27ac, H3k27me3), chromatin accessibility (e.g., DNase-seq or ATAC-seq) or DNA methylation. Second, based on the recovered regions and a list of known TF motifs, regions are searched for enriched patterns and their significance evaluated. Third, integrating gene expression data a short list of candidate TF are provided to account for their endogenous expression across the different cell types and their expected potency based on genes that are downstream of the regions uncovered in the second step. Finally, a ranked list of TF sequences is also generated based on this integrative approach for each cell type.

As previously described (Pinello et al. Bioinformatics 2018) by exploiting chromatin accessibility (or histone marks) and gene expression variability across cell types key regulatory regions and regulators were extracted that are cell type specific. By the integration of available data from large consortia (Roadmap Epigenomic and ENCODE projects) and using Haystack (github.com/pinellolab/haystack bio) we have curated a list of regions and transcription factors for several human primary cells and cell lines. Importantly, this strategy can be applied to any organism for which a reference genome is available.

In some embodiments, the discovery of the sequence motifs is done by de novo motif discovery analysis within a single cell type or set of cell types of interest.

In some embodiments, the discovery of sequence motifs is done by analyzing cis-regulatory DNA sequence composition of top-expressing genes (e.g., top 1%, 5%, 20%, 50%) ranked by normalized expression values (e.g., RPKM, FPKM, TPM, fold-change) in a single cell type or set of cell types of interest.

In some embodiments, the discovery of the sequence motifs is done by analyzing cis-regulatory DNA sequence composition of bottom-expressing genes (e.g., bottom 1%, 5%, 20%, 50%) ranked by normalized expression values (e.g., RPKM, FPKM, TPM, fold-change) in a single cell type or set of cell types of interest.

In some embodiments, the discovery of the sequence motifs is done through frequency-based methods including the construction of position-weight matrices for a single cell type or set of cell types of interest.

In some embodiments, the discovery of the sequence motifs is done through neural network architectures to identify sequence motifs that may or may not exist in a single cell type or set of cell types of interest.

In some embodiments, the discovery of the sequence motifs is done through generation of synthetic DNA sequences using language models—a generative deep learning technique—where the sequence motifs may or may not exist in a single cell type or set of cell types of interest.

In some embodiments, the discovery of the sequence motifs is done through generation of synthetic DNA sequences using deep variational autoencoders—a generative deep learning model—where the sequence motifs may or may not exist in a single cell type or set of cell types of interest.

In some embodiments, the discovery of the sequence motifs is done through generation of synthetic DNA sequences using Generative Adversarial Networks (GANs)—a generative deep learning model—where the sequence motifs may or may not exist in a single cell type or set of cell types of interest.

In some embodiments, the discovery of the sequence motifs is done through the identification of transcription factor binding sequence motifs with dependencies with other sequence motifs (e.g., pairwise, triwise interactions)¹⁶.

In some embodiments, the discovery of the sequence motifs is done through the identification of transcription factor binding sequence motifs that recruit additional transcriptional machinery through protein-protein interactions.

In addition, a number of regulatory sequence motifs, e.g., transcription factors and transcription factor binding sequence motifs, are known, including those that are cell-type specific and those that are hormone responsive. For example, Table 3A provides a list of TFs and their entry number in the JASPAR database, which provides their recognition sequences.

Activating target genes in a cell-type-specific manner can be achieved by recruiting cell-type specific endogenous TFs (many of which are known in the art) at the promoters, enhancers or both of genes of interest. First, a list of cell-type-specific TFs for the target cell lines can be determined based on the expression levels from RNA-seq of native cell lines³³. Second, individual or repeats of a cell-type specific TF motif or combination of multiple TF motifs can be introduced at the promoters, enhancers or both of genes of interest in the target cell lines via genetic modifiers. For controls, cell lines that do not or lowly express cell-type-specific TFs that express in target cell lines should be tested to see if target genes are not expressed even though same genomic modifications were installed.

A number of databases provide lists of cell-specific transcription factors, including TRANSFAC and TFregulome®; see also D'Alessio, A. C. et al. A Systematic Approach to Identify Candidate Transcription Factors that Control Cell Identity. Stem Cell Reports 5, 763-Instead of the recruitment of endogenous transcription factors, SHAPE can also be used to introduce de novo transcription factor binding sites that act as response elements for TFs or TF-like proteins that can be activated (e.g., by expression or reduced degradation) by an exogenous small molecule, hormone or drug following the subsequent addition of the exogenous small molecule, hormone, or drug. Upon induction of this exogenous ligand, the response element would recruit the receptor-exogenous ligand complexes to the target locus and give rise to target gene activation. Examples include the use of vitamin D that interact with vitamin D receptor (VDR) transcription factor by installing VDR response motif at the promoters or enhancers at target genes of interest.

Table 3B provides a list of examples of hormone responsive transcription factors and elements.

TABLE 3A
Examples of transcription factors from the JASPAR database that
may be recruited using SHAPE

| JASPAR ID | Transcription factor |
|---|---|
| MA0206.1 | abd-A |
| MA0165.1 | Abd-B |
| MA0265.1 | ABF1 |
| MA0570.2 | ABF1 |
| MA0266.1 | ABF2 |
| MA0941.1 | ABF2 |
| MA0930.1 | ABF3 |
| MA1659.1 | ABF4 |
| MA0564.1 | ABI3 |
| MA0123.1 | abi4 |
| MA0931.1 | ABI5 |
| MA1244.1 | ABR1 |
| MA0267.1 | ACE2 |
| MA0207.1 | achi |
| MA1277.1 | Adof1 |
| MA0268.1 | ADR1 |
| MA0269.1 | AFT1 |
| MA0270.1 | AFT2 |
| MA0005.2 | AG |
| MA0585.1 | AGL1 |
| MA1204.1 | AGL13 |
| MA0548.2 | AGL15 |
| MA1199.1 | AGL16 |
| MA1200.1 | AGL25 |
| MA1012.1 | AGL27 |
| MA0001.2 | AGL3 |
| MA1201.1 | AGL42 |
| MA1202.1 | AGL55 |
| MA1205.1 | AGL6 |
| MA1203.1 | AGL63 |
| MA0932.1 | AHL12 |
| MA0933.1 | AHL20 |
| MA0934.1 | AHL25 |
| MA0006.1 | Ahr::Arnt |
| MA0959.1 | AIB |
| MA1235.1 | AIL7 |
| MA0208.1 | al |
| MA0854.1 | Alx1 |
| MA0634.1 | ALX3 |
| MA0853.1 | Alx4 |
| MA1663.1 | ANAC050 |
| MA1660.1 | ANAC13 |
| MA1375.1 | ANL2 |
| MA0571.1 | ANT |
| MA0166.1 | Antp |
| MA0209.1 | ap |
| MA0940.1 | AP1 |
| MA0556.1 | AP3 |
| MA0007.3 | Ar |
| MA0210.1 | ara |
| MA1098.1 | ARALYDRAFT_484486 |
| MA1097.1 | ARALYDRAFT_493022 |
| MA1095.1 | ARALYDRAFT_495258 |
| MA1096.1 | ARALYDRAFT_496250 |
| MA1054.1 | ARALYDRAFT_897773 |
| MA1338.1 | AREB3 |
| MA0942.1 | ARF1 |
| MA1685.1 | ARF10 |
| MA1686.1 | ARF13 |
| MA1687.1 | ARF14 |
| MA1688.1 | ARF16 |
| MA1689.1 | ARF18 |
| MA1206.1 | ARF2 |
| MA1690.1 | ARF25 |
| MA1691.1 | ARF27 |
| MA1692.1 | ARF29 |
| MA1009.1 | ARF3 |
| MA1693.1 | ARF34 |
| MA1694.1 | ARF35 |
| MA1695.1 | ARF36 |
| MA1696.1 | ARF39 |
| MA1697.1 | ARF4 |
| MA0943.1 | ARF5 |
| MA1698.1 | ARF7 |
| MA0944.1 | ARF8 |
| MA0271.1 | ARG80 |
| MA0272.1 | ARG81 |
| MA1463.1 | ARGFX |
| MA0151.1 | Arid3a |
| MA0601.1 | Arid3b |
| MA0602.1 | Arid5a |
| MA0004.1 | Arnt |
| MA0259.1 | ARNT::HIF1A |
| MA1464.1 | ARNT2 |
| MA0603.1 | Amtl |
| MA0273.1 | ARO80 |
| MA0274.1 | ARR1 |
| MA0945.1 | ARR1 |
| MA0121.1 | ARR10 |
| MA0946.1 | ARR11 |
| MA0947.1 | ARR14 |
| MA0948.1 | ARR18 |
| MA0949.1 | ARR2 |
| MA0874.1 | Arx |
| MA1100.2 | ASCL1 |
| MA1631.1 | ASCL1(var. 2) |
| MA0816.1 | Ascl2 |
| MA0275.1 | ASG1 |
| MA0276.1 | ASH1 |
| MA1661.1 | AT-GTL1 |
| MA1259.1 | AT1G01250 |
| MA1387.1 | AT1G13300 |
| MA1160.1 | AT1G14580 |
| MA1400.1 | At1g19000 |
| MA1255.1 | AT1G22810 |
| MA1263.1 | AT1G36060 |
| MA1222.1 | AT1G44830 |
| MA1275.1 | AT1G47655 |
| MA1186.1 | At1g49010 |
| MA1268.1 | AT1G69570 |
| MA1288.1 | At1g72010 |
| MA1353.1 | AT1G72740 |
| MA1397.1 | At1g74840 |
| MA1250.1 | AT1G75490 |
| MA1367.1 | AT1G76870 |
| MA1366.1 | AT1G76880 |
| MA1260.1 | AT1G77640 |
| MA1380.1 | AT2G20110 |
| MA1272.1 | AT2G28810 |
| MA1245.2 | AT2G33710 |
| MA1193.1 | At2g38090 |
| MA1385.1 | AT2G40260 |
| MA1229.1 | AT2G44940 |
| MA1285.1 | At2g45680 |
| MA1182.1 | At3g09600 |
| MA1662.1 | AT3G10030 |
| MA1191.1 | AT3G10113 |
| MA1194.1 | AT3G10580 |
| MA1188.1 | At3g11280 |
| MA1253.1 | AT3G16280 |
| MA1388.1 | AT3G24120 |
| MA1270.1 | AT3G45610 |
| MA1381.1 | AT3G46070 |
| MA1276.1 | AT3G52440 |
| MA1242.1 | AT3G57600 |
| MA1354.1 | AT4G12670 |
| MA1237.1 | AT4G16750 |
| MA1228.1 | AT4G18450 |
| MA1232.1 | AT4G28140 |
| MA1241.1 | AT4G32800 |
| MA1281.1 | AT5G02460 |
| MA1196.1 | At5g05790 |
| MA1399.1 | At5g08520 |
| MA1226.1 | AT5G18450 |
| MA1365.2 | AT5G47660 |
| MA1195.1 | AT5G56840 |
| MA1192.1 | At5g58900 |
| MA1189.1 | AT5G61620 |
| MA1267.1 | AT5G66940 |
| MA1261.1 | AT5G67000 |
| MA1438.1 | atf-7 |
| MA0604.1 | Atf1 |
| MA1632.1 | ATF2 |
| MA0605.2 | ATF3 |
| MA0833.2 | ATF4 |
| MA1466.1 | ATF6 |
| MA834.1 | ATF7 |
| MA0950.1 | ATHB-12 |
| MA0951.1 | ATHB-16 |
| MA0110.3 | ATHB-5 |
| MA0952.1 | ATHB-51 |
| MA0953.1 | ATHB-6 |
| MA0573.1 | ATHB-9 |
| MA1212.1 | ATHB13 |
| MA1026.2 | ATHB15 |
| MA1211.1 | ATHB18 |
| MA1209.1 | ATHB20 |
| MA1327.2 | ATHB23 |
| MA1328.1 | ATHB34 |
| MA1406.1 | ATHB4 |
| MA1214.1 | ATHB40 |
| MA1215.1 | ATHB53 |
| MA0954.2 | ATHB7 |
| MA0461.2 | Atoh1 |
| MA1467.1 | ATOH1(var. 2) |
| MA1468.1 | ATOH7 |
| MA0167.1 | Awh |
| MA0277.1 | AZF1 |
| MA0168.1 | B-H1 |
| MA0169.1 | B-H2 |
| MA1633.1 | BACH1 |
| MA0591.1 | Bach1::Mafk |
| MA1101.2 | BACH 2 |
| MA1470.1 | BACH2(var. 2) |
| MA0211.1 | bap |
| MA0877.2 | BARHL1 |
| MA0635.1 | BARHL2 |
| MA0875.1 | BARX1 |
| MA1471.1 | BARX2 |
| MA0278.1 | BAS1 |
| MA1634.1 | BATF |
| MA0462.2 | BATF::JUN |
| MA0835.2 | BATF3 |
| MA0212.1 | bcd |
| MA0463.2 | BCL6 |
| MA0731.1 | BCL6B |
| MA0529.2 | BEAF-32 |
| MA0956.1 | BEE2 |
| MA1332.1 | BEH2 |
| MA1333.1 | BEH3 |
| MA1331.1 | BEH4 |
| MA0242.1 | Bgb::run |
| MA0960.1 | BHLH104 |
| MA0961.1 | BHLH112 |
| MA0958.1 | BHLH13 |
| MA1358.1 | bHLH130 |
| MA1361.1 | bHLH18 |
| MA0957.1 | BHLH3 |
| MA1359.1 | bHLH31 |
| MA0962.1 | BHLH34 |
| MA1363.1 | bHLH69 |
| MA1360.1 | bHLH74 |
| MA1362.1 | bHLH77 |
| MA0963.1 | BHLH78 |
| MA1357.1 | bHLH80 |
| MA0607.1 | Bhlha15 |
| MA1472.1 | BHLHA15(var. 2) |
| MA0818.1 | BHLHE22 |
| MA1635.1 | BHLHE22(var. 2) |
| MA0817.1 | BHLHE23 |
| MA0464.2 | BHLHE40 |
| MA0636.1 | BHLHE41 |
| MA0964.1 | BIM1 |
| MA0965.1 | BIM2 |
| MA0966.1 | BIM3 |
| MA0537.1 | blmp-1 |
| MA1404.1 | BPC1 |
| MA1403.1 | BPC5 |
| MA1402.1 | BPC6 |
| MA0010.1 | br |
| MA0011.1 | br(var. 2) |
| MA0012.1 | br(var. 3) |
| MA0013.1 | br(var. 4) |
| MA0213.1 | brk |
| MA0214.1 | bsh |
| MA0876.1 | BSX |
| MA0443.1 | btd |
| MA0215.1 | btn |
| MA1407.1 | bZIP14 |
| MA1349.1 | bZIP16 |
| MA1344.1 | bZIP28 |
| MA1340.1 | bZIP3 |
| MA1350.1 | bZIP42 |
| MA1339.1 | bZIP43 |
| MA1337.1 | bZIP44 |
| MA1345.1 | bZIP48 |
| MA1342.1 | bZIP50 |
| MA1343.1 | bZIP52 |
| MA1341.1 | bZIP53 |
| MA0967.1 | BZIP60 |
| MA0968.2 | bZIP68 |
| MA0096.1 | bZIP910 |
| MA0097.1 | bZIP911 |
| MA0550.2 | BZR1 |
| MA0549.1 | BZR2 |
| MA0170.1 | C15 |
| MA0216.2 | cad |
| MA0279.1 | CAD1 |
| MA1197.1 | CAMTA1 |
| MA0280.1 | CAT8 |
| MA0217.1 | caup |
| MA0281.1 | CBF1 |
| MA1224.1 | CBF1 |
| MA1217.1 | CBF2 |
| MA1218.1 | CBF4 |
| MA0972.1 | CCA1 |
| MA0579.1 | CDC5 |
| MA0973.1 | CDF2 |
| MA0974.2 | CDF3 |
| MA0878.2 | CDX1 |
| MA0465.2 | CDX2 |
| MA1473.1 | CDX4 |
| MA1444.1 | cebp-1 |
| MA0102.4 | CEBPA |
| MA0466.2 | CEBPB |
| MA0836.2 | CEBPD |
| MA0837.1 | CEBPE |
| MA0838.1 | CEBPG |
| MA1636.1 | CEBPG(var. 2) |
| MA0263.1 | ceh-10::ttx-3 |
| MA0264.1 | ceh-22 |
| MA1445.1 | ceh-28 |
| MA1699.1 | ceh-38 |
| MA0921.1 | ceh-48 |
| MA1219.1 | CEJ1 |
| MA0637.1 | CENPB |
| MA0282.1 | CEP3 |
| MA0922.1 | ces-2 |
| MA0015.1 | Cf2 |
| MA0171.1 | CG11085 |
| MA0172.1 | CG11294 |
| MA0173.1 | CG11617 |
| MA0176.1 | CG15696-RA |
| MA0177.1 | CG18599 |
| MA0178.1 | CG32105 |
| MA0179.1 | CG32532 |
| MA0444.1 | CG34031 |
| MA0182.1 | CG4328-RA |
| MA0184.1 | CG9876 |
| MA0283.1 | CHA4 |
| MA0260.1 | che-1 |
| MA0284.1 | CIN5 |
| MA1700.1 | Clamp |
| MA0819.1 | CLOCK |
| MA0969.1 | CMTA2 |
| MA0970.1 | CMTA3 |
| MA0530.1 | cnc::maf-S |
| MA1279.1 | COG1 |
| MA1432.1 | cre-1 |
| MA0018.4 | CREB1 |
| MA0638.1 | CREB3 |
| MA0839.1 | CREB3L1 |
| MA0608.1 | Creb3l2 |
| MA1474.1 | CREB3L4 |
| MA1475.1 | CREB3L4(var. 2) |
| MA0840.1 | Creb5 |
| MA0609.2 | CREM |
| MA0975.1 | CRF2 |
| MA0976.2 | CRF4 |
| MA0467.1 | Crx |
| MA0285.1 | CRZ1 |
| MA0286.1 | CST6 |
| MA0218.1 | Ct |
| MA0139.1 | CTCF |
| MA0531.1 | CTCF |
| MA1102.2 | CTCFL |
| MA0287.1 | CUP2 |
| MA0288.1 | CUP9 |
| MA0754.1 | CUX1 |
| MA0755.1 | CUX2 |
| MA0445.1 | D |
| MA0538.1 | daf-12 |
| MA1446.1 | daf-16 |
| MA1271.1 | DAG2 |
| MA0289.1 | DAL80 |
| MA0290.1 | DAL81 |
| MA0291.1 | DAL82 |
| MA0639.1 | DBP |
| MA0174.1 | Dbx |
| MA0019.1 | Ddit3::Cebpa |
| MA0185.1 | Deaf1 |
| MA1376.1 | DEAR3 |
| MA1251.1 | DEAR5 |
| MA0186.1 | Dfd |
| MA0022.1 | dl |
| MA0023.1 | dl(var. 2) |
| MA0187.1 | Dll |
| MA0879.1 | Dlx1 |
| MA0885.1 | Dlx2 |
| MA0880.1 | Dlx3 |
| MA0881.1 | Dlx4 |
| MA1476.1 | DLX5 |
| MA0882.1 | DLX6 |
| MA0883.1 | Dmbx1 |
| MA1603.1 | Dmrt1 |
| MA0610.1 | DMRT3 |
| MA1455.1 | dmrt99B |
| MA1478.1 | DMRTA2 |
| MA1479.1 | DMRTC2 |
| MA0981.1 | DOF1.8 |
| MA0020.1 | Dof2 |
| MA0982.1 | DOF2.4 |
| MA0977.1 | DOF2.5 |
| MA0021.1 | Dof3 |
| MA1273.1 | dof4.2 |
| MA1269.1 | dof4.5 |
| MA1071.1 | DOF5.3 |
| MA0983.1 | DOF5.6 |
| MA0984.1 | DOF5.7 |
| MA0351.1 | DOT6 |
| MA1668.1 | DPBF3 |
| MA1480.1 | DPRX |
| MA0540.1 | dpy-27 |
| MA0188.1 | Dr |
| MA0985.1 | DRE1C |
| MA1243.1 | DREB19 |
| MA0971.1 | DREB1A |
| MA1669.1 | DREB1B |
| MA1670.1 | DREB1C |
| MA0978.1 | DREB1E |
| MA1032.1 | DREB1G |
| MA1258.1 | DREB2 |
| MA1248.1 | DREB26 |
| MA0986.1 | DREB2C |
| MA1456.1 | Dref |
| MA1481.1 | DRGX |
| MA0919.1 | dsc-1 |
| MA0611.1 | Dux |
| MA0468.1 | DUX4 |
| MA0884.1 | DUXA |
| MA0915.1 | dve |
| MA0580.1 | DYT1 |
| MA0024.3 | E2F1 |
| MA0864.2 | E2F2 |
| MA0469.3 | E2F3 |
| MA0470.2 | E2F4 |
| MA0471.2 | E2F6 |
| MA0758.1 | E2F7 |
| MA0865.1 | E2F8 |
| MA1414.1 | E2FA |
| MA0189.1 | E5 |
| MA0154.4 | EBF1 |
| MA1604.1 | Ebf2 |
| MA1637.1 | EBF3 |
| MA0292.1 | ECM22 |
| MA0293.1 | ECM23 |
| MA0534.1 | EcR::usp |
| MA0294.1 | EDS1 |
| MA0990.1 | EDT1 |
| MA0541.1 | efl-1 |
| MA1167.1 | EFM |
| MA0162.4 | EGR1 |
| MA0472.2 | EGR2 |
| MA0732.1 | EGR3 |
| MA0733.1 | EGR4 |
| MA0598.3 | EHF |
| MA0026.1 | Eip74EF |
| MA0473.3 | ELF1 |
| MA1483.1 | ELF2 |
| MA0640.2 | ELF3 |
| MA0641.1 | ELF4 |
| MA0136.2 | ELF5 |
| MA0028.2 | ELK1 |
| MA0759.1 | ELK3 |
| MA0076.2 | ELK4 |
| MA1701.1 | elt-2 |
| MA0542.1 | elt-3 |
| MA1439.1 | elt-6 |
| MA0128.1 | EmBP-1 |
| MA0219.1 | ems |
| MA0612.2 | EMXI |
| MA0886.1 | EMX2 |
| MA0220.1 | en |
| MA0027.2 | EN1 |
| MA0642.1 | EN2 |
| MA0800.1 | EOMES |
| MA0543.1 | eor-1 |
| MA1401.1 | EPR1 |
| MA0760.1 | ERF |
| MA0979.1 | ERF008 |
| MA1265.2 | ERF015 |
| MA1234.1 | ERF017 |
| MA1048.1 | ERF018 |
| MA1424.1 | ERF019 |
| MA1233.2 | ERF021 |
| MA1227.2 | ERF027 |
| MA1223.1 | ERF035 |
| MA1230.1 | ERF037 |
| MA0995.2 | ERF039 |
| MA0996.1 | ERF043 |
| MA0997.1 | ERF069 |
| MA1247.1 | ERF087 |
| MA1049.1 | ERF094 |
| MA0998.1 | ERF096 |
| MA0999.1 | ERF098 |
| MA1240.1 | ERF10 |
| MA1239.1 | ERF104 |
| MA1000.2 | ERF105 |
| MA1053.1 | ERF109 |
| MA1001.2 | ERF11 |
| MA1002.1 | ERF112 |
| MA1671.1 | ERF118 |
| MA1004.1 | ERF13 |
| MA1231.1 | ERF15 |
| MA0567.1 | ERF1B |
| MA1262.1 | ERF2 |
| MA1005.2 | ERF3 |
| MA1238.2 | ERF38 |
| MA0992.2 | ERF4 |
| MA1225.1 | ERF5 |
| MA1006.1 | ERF6 |
| MA0993.1 | ERF7 |
| MA0994.1 | ERF8 |
| MA1257.1 | ERF9 |
| MA0474.2 | ERG |
| MA0420.1 | ERT1 |
| MA1264.1 | ESE1 |
| MA1236.1 | ESE3 |
| MA0112.3 | ESR1 |
| MA0258.2 | ESR2 |
| MA0592.3 | ESRRA |
| MA0141.3 | ESRRB |
| MA0643.1 | Esrrg |
| MA0644.1 | ESX1 |
| MA0098.3 | ETS1 |
| MA1484.1 | ETS2 |
| MA0916.1 | Ets21C |
| MA0761.2 | ETV1 |
| MA0762.1 | ETV2 |
| MA0763.1 | ETV3 |
| MA0764.2 | ETV4 |
| MA0765.2 | ETV5 |
| MA0645.1 | ETV6 |
| MA0221.1 | eve |
| MA0887.1 | EVX1 |
| MA0888.1 | EVX2 |
| MA0149.1 | EWSR1-FLI1 |
| MA0222.1 | exd |
| MA0224.1 | exex |
| MA0575.1 | F3A4.140 |
| MA1408.1 | FaEOBII |
| MA1382.1 | FAR1 |
| MA1485.1 | FERD3L |
| MA0156.2 | FEV |
| MA0295.1 | FHL1 |
| MA0557.1 | FHY3 |
| MA0820.1 | FIGLA |
| MA0446.1 | fkh |
| MA0920.1 | fkh-2 |
| MA1440.1 | fkh-9 |
| MA0296.1 | FKH1 |
| MA0297.1 | FKH2 |
| MA0558.1 | FLC |
| MA0475.2 | FLI1 |
| MA0476.1 | FOS |
| MA1448.1 | fos-1 |
| MA0099.3 | FOS::JUN |
| MA1126.1 | FOS::JUN(var. 2) |
| MA1134.1 | FOS::JUNB |
| MA1141.1 | FOS::JUND |
| MA1127.1 | FOSB::JUN |
| MA1135.1 | FOSB::JUNB |
| MA1136.1 | FOSB::JUNB(var. 2) |
| MA0477.2 | FOSL1 |
| MA1128.1 | FOSL1::JUN |
| MA1129.1 | FOSLl::JUN(var. 2) |
| MA1137.1 | FOSL1::JUNB |
| MA1142.1 | FOSL1::JUND |
| MA1143.1 | FOSLl::JUND(var. 2) |
| MA0478.1 | FOSL2 |
| MA1130.1 | FOSL2::JUN |
| MA1131.1 | FOSL2::JUN(var. 2) |
| MA1138.1 | FOSL2::JUNB |
| MA1139.1 | FOSL2::JUNB(var. 2) |
| MA1144.1 | FOSL2::JUND |
| MA1145.1 | FOSL2::JUND(var. 2) |
| MA0148.4 | FOXA1 |
| MA0047.3 | FOXA2 |
| MA1683.1 | FOXA3 |
| MA0845.1 | FOXB1 |
| MA0032.2 | FOXC1 |
| MA0846.1 | FOXC2 |
| MA0031.1 | FOXD1 |
| MA0847.2 | FOXD2 |
| MA0041.1 | Foxd3 |
| MA1487.1 | FOXE1 |
| MA1606.1 | Foxf1 |
| MA0030.1 | FOXF2 |
| MA0613.1 | FOXG1 |
| MA0479.1 | FOXH1 |
| MA0042.2 | FOXl1 |
| MA0614.1 | Foxj2 |
| MA0851.1 | Foxj3 |
| MA0852.2 | FOXK1 |
| MA1103.2 | FOXK2 |
| MA0033.2 | FOXL1 |
| MA1607.1 | Foxl2 |
| MA1684.1 | Foxn1 |
| MA1489.1 | FOXN3 |
| MA0480.1 | Foxo1 |
| MA0157.2 | FOXO3 |
| MA0848.1 | FOXO4 |
| MA0849.1 | FOXO6 |
| MA0481.3 | FOXP1 |
| MA0593.1 | FOXP2 |
| MA0850.1 | FOXP3 |
| MA0040.1 | Foxq1 |
| MA0225.1 | ftz |
| MA0565.2 | FUS3 |
| MA0298.1 | FZF1 |
| MA0062.3 | GABPA |
| MA0299.1 | GAL4 |
| MA0034.1 | Gam1 |
| MA0300.1 | GAT1 |
| MA0301.1 | GAT3 |
| MA0302.1 | GAT4 |
| MA0035.4 | GATA1 |
| MA0140.2 | GATA1::TAL1 |
| MA1013.1 | GATA10 |
| MA1014.1 | GATA11 |
| MA1015.1 | GATA12 |
| MA1325.1 | GATA14 |
| MA1016.1 | GATA15 |
| MA1323.1 | GATA19 |
| MA0036.3 | GATA2 |
| MA1324.1 | GATA20 |
| MA0037.3 | GATA3 |
| MA0482.2 | GATA4 |
| MA0766.2 | GATA5 |
| MA1396.1 | GATA6 |
| MA1104.2 | GATA6 |
| MA1017.1 | GATA8 |
| MA1018.1 | GATA9 |
| MA1672.1 | GBF2 |
| MA1351.1 | GBF3 |
| MA1334.1 | GBF6 |
| MA0889.1 | GBX1 |
| MA0890.1 | GBX2 |
| MA0646.1 | GCM1 |
| MA0767.1 | GCM2 |
| MA0917.1 | gcm2 |
| MA0303.1 | GCN4 |
| MA0304.1 | GCR1 |
| MA0305.1 | GCR2 |
| MA0038.2 | GFl1 |
| MA0483.1 | Gfi1b |
| MA0306.1 | GIS1 |
| MA0734.2 | GLI2 |
| MA1491.1 | GLI3 |
| MA0735.1 | GLIS1 |
| MA0736.1 | GLIS2 |
| MA0737.1 | GLIS3 |
| MA0307.1 | GLN3 |
| MA1019.1 | Glyma19g26560.1 |
| MA0615.1 | Gmeb1 |
| MA0862.1 | GMEB2 |
| MA1457.1 | grh |
| MA0647.1 | GRHL1 |
| MA1105.2 | GRHL2 |
| MA0190.1 | Gsc |
| MA0648.1 | GSC |
| MA0891.1 | GSC2 |
| MA0308.1 | GSM1 |
| MA0892.1 | GSX1 |
| MA0893.2 | GSX2 |
| MA0447.1 | gt |
| MA1020.1 | GT-1 |
| MA1208.1 | GT-2 |
| MA1368.2 | GT-4 |
| MA1207.1 | GT-A3 |
| MA0309.1 | GZF3 |
| MA0449.1 | h |
| MA0448.1 | H2.0 |
| MA0310.1 | HAC1 |
| MA0311.1 | HAL9 |
| MA0092.1 | Hand1::Tcf3 |
| MA1638.1 | HAND2 |
| MA0312.1 | HAP1 |
| MA0313.1 | HAP2 |
| MA0314.1 | HAP3 |
| MA0316.1 | HAP5 |
| MA1024.1 | HAT1 |
| MA1198.1 | HAT2 |
| MA1210.1 | HAT22 |
| MA0008.2 | HAT5 |
| MA0049.1 | hb |
| MA1025.1 | HBI1 |
| MA0226.1 | hbn |
| MA0317.1 | HCM1 |
| MA1369.1 | HDG1 |
| MA1099.2 | HES1 |
| MA0616.2 | HES2 |
| MA0821.1 | HES5 |
| MA1493.1 | HES6 |
| MA0822.1 | HES7 |
| MA0894.1 | HESX1 |
| MA0823.1 | HEY1 |
| MA0649.1 | HEY2 |
| MA0191.1 | HGTX |
| MA0183.1 | HHEX |
| MA1390.1 | HHO2 |
| MA1386.1 | HHO3 |
| MA1164.1 | HHO5 |
| MA1165.1 | HHO6 |
| MA0739.1 | Hic1 |
| MA0738.1 | HIC2 |
| MA1106.1 | HIF1A |
| MA0131.2 | HINFP |
| MA0450.1 | hkb |
| MA0043.3 | HLF |
| MA0545.1 | hlh-1 |
| MA1449.1 | hlh-30 |
| MA0109.1 | HLTF |
| MA0895.1 | HMBOX1 |
| MA0044.1 | HMG-1 |
| MA0045.1 | HMG-I/Y |
| MA0327.1 | HMRAl |
| MA0318.1 | HMRA2 |
| MA0192.1 | Hmx |
| MA0896.1 | Hmx1 |
| MA0897.1 | Hmx2 |
| MA0898.1 | Hmx3 |
| MA0046.2 | HNF1A |
| MA0153.2 | HNF1B |
| MA0114.4 | HNF4A |
| MA1494.1 | HNF4A(var. 2) |
| MA0484.2 | HNF4G |
| MA1495.1 | HOXA1 |
| MA0899.1 | HOXA10 |
| MA0911.1 | Hoxa11 |
| MA0650.2 | HOXA13 |
| MA0900.2 | HOXA2 |
| MA1496.1 | HOXA4 |
| MA0158.2 | HOXA5 |
| MA1497.1 | HOXA6 |
| MA1498.1 | HOXA7 |
| MA0594.2 | HOXA9 |
| MA0901.2 | HOXB13 |
| MA0902.2 | HOXB2 |
| MA0903.1 | HOXB3 |
| MA1499.1 | HOXB4 |
| MA0904.2 | HOXB5 |
| MA1500.1 | HOXB6 |
| MA1501.1 | HOXB7 |
| MA1502.1 | HOXB8 |
| MA1503.1 | HOXB9 |
| MA0905.1 | HOXC10 |
| MA0651.1 | HOXC11 |
| MA0906.1 | HOXC12 |
| MA0907.1 | HOXC13 |
| MA1504.1 | HOXC4 |
| MA1505.1 | HOXC8 |
| MA0485.2 | HOXC9 |
| MA1506.1 | HOXD10 |
| MA0908.1 | HOXD11 |
| MA0873.1 | HOXD12 |
| MA0909.2 | HOXD13 |
| MA0912.2 | HOXD3 |
| MA1507.1 | HOXD4 |
| MA0910.2 | HOXD8 |
| MA0913.2 | HOXD9 |
| MA1458.1 | Hsf |
| MA0319.1 | HSF1 |
| MA0486.2 | HSF1 |
| MA0770.1 | HSF2 |
| MA0771.1 | HSF4 |
| MA1664.1 | HSFA6B |
| MA1665.1 | HSFB2A |
| MA1666.1 | HSFB2B |
| MA1667.1 | HSFC1 |
| MA0227.1 | hth |
| MA0551.1 | HY5 |
| MA1425.1 | HYH |
| MA0120.1 | id1 |
| MA1373.1 | IDD2 |
| MA1371.1 | IDD4 |
| MA1370.1 | IDD5 |
| MA1374.1 | IDD7 |
| MA1508.1 | IKZF1 |
| MA0320.1 | IME1 |
| MA0228.1 | ind |
| MA0321.1 | INO2 |
| MA0322.1 | INO4 |
| MA0155.1 | INSM1 |
| MA0229.1 | inv |
| MA0050.2 | IRF1 |
| MA0051.1 | IRF2 |
| MA1418.1 | IRF3 |
| MA1419.1 | IRF4 |
| MA1420.1 | IRF5 |
| MA1509.1 | IRF6 |
| MA0772.1 | IRF7 |
| MA0652.1 | IRF8 |
| MA0653.1 | IRF9 |
| MA1608.1 | Isl1 |
| MA0914.1 | ISL2 |
| MA0654.1 | ISX |
| MA0323.1 | IXRI |
| MA0655.1 | JDP2 |
| MA0656.1 | JDP2(var. 2) |
| MA1156.1 | JKD |
| MA0488.1 | JUN |
| MA1132.1 | JUN::JUNB |
| MA1133.1 | JUN::JUNB(var. 2) |
| MA0489.1 | JUN(var. 2) |
| MA0490.2 | JUNB |
| MA1140.2 | JUNB(var. 2) |
| MA0491.2 | JUND |
| MA0492.1 | JUND(var. 2) |
| MA1027.1 | KAN1 |
| MA1383.1 | KAN2 |
| MA1028.1 | KAN4 |
| MA0493.1 | KIf1 |
| MA1511.1 | KLF10 |
| MA1512.1 | KLF11 |
| MA0742.1 | Klf12 |
| MA0657.1 | KLF13 |
| MA0740.1 | KLF14 |
| MA1513.1 | KLF15 |
| MA0741.1 | KLF16 |
| MA1514.1 | KLF17 |
| MA1515.1 | KLF2 |
| MA1516.1 | KLF3 |
| MA0039.4 | KLF4 |
| MA0599.1 | KLF5 |
| MA1517.1 | KLF6 |
| MA1107.2 | KLF9 |
| MA0451.1 | kni |
| MA0452.2 | Kr |
| MA1398.2 | KUA1 |
| MA0230.1 | lab |
| MA1673.1 | LBD18 |
| MA0231.1 | Ibe |
| MA0232.1 | Ibl |
| MA0618.1 | LBX1 |
| MA0699.1 | LBX2 |
| MA1187.1 | LCL1 |
| MA0581.1 | LEC2 |
| MA0768.1 | LEF1 |
| MA1246.1 | LEP |
| MA0324.1 | LEU3 |
| MA0590.1 | LFY |
| MA1518.1 | LHX1 |
| MA0700.2 | LHX2 |
| MA0135.1 | Lhx3 |
| MA0704.1 | Lhx4 |
| MA1519.1 | LHX5 |
| MA0658.1 | LHX6 |
| MA0705.1 | Lhx8 |
| MA0701.2 | LHX9 |
| MA1185.1 | LHY1 |
| MA0923.1 | lim-4 |
| MA1441.1 | lim-7 |
| MA0194.1 | Lim1 |
| MA0195.1 | Lim3 |
| MA0261.1 | lin-14 |
| MA1450.1 | lin-54 |
| MA0619.1 | LIN54 |
| MA0175.1 | Ims |
| MA0702.2 | LMX1A |
| MA0703.2 | LMX1B |
| MA0325.1 | LYS14 |
| MA1459.1 | M1BP |
| MA0262.1 | mab-3 |
| MA1442.1 | mab-5 |
| MA0326.1 | MAC1 |
| MA0118.1 | Macho-1 |
| MA0535.1 | Mad |
| MA1520.1 | MAF |
| MA0501.1 | MAF::NFE2 |
| MA1521.1 | MAFA |
| MA0117.2 | Mafb |
| MA0495.3 | MAFF |
| MA0659.2 | MAFG |
| MA0496.3 | MAFK |
| MA0328.2 | MATALPHA2 |
| MA0058.3 | MAX |
| MA0059.1 | MAX::MYC |
| MA1522.1 | MAZ |
| MA0329.1 | MBP1 |
| MA0330.1 | MBP1::SWI6 |
| MA0331.1 | MCM1 |
| MA0029.1 | Mecom |
| MA0052.4 | MEF2A |
| MA0660.1 | MEF2B |
| MA0497.1 | MEF2C |
| MA0773.1 | MEF2D |
| MA0498.2 | MEIS1 |
| MA1639.1 | MEIS1(var. 2) |
| MA0774.1 | MEIS2 |
| MA1640.1 | MEIS2(var. 2) |
| MA0775.1 | MEIS3 |
| MA0661.1 | MEOX1 |
| MA0706.1 | MEOX2 |
| MA0332.1 | MET28 |
| MA0333.1 | MET31 |
| MA0334.1 | MET32 |
| MA0335.1 | MET4 |
| MA0801.1 | MGA |
| MA0336.1 | MGA1 |
| MA1158.1 | MGP |
| MA0337.1 | MIG1 |
| MA0338.1 | MIG2 |
| MA0339.1 | MIG3 |
| MA0233.1 | mirr |
| MA0620.3 | MITF |
| MA0621.1 | mix-a |
| MA0662.1 | MIXL1 |
| MA0663.1 | MLX |
| MA0622.1 | MIxip |
| MA0664.1 | MLXIPL |
| MA0053.1 | MNB1A |
| MA0825.1 | MNT |
| MA0707.1 | MNX1 |
| MA0379.1 | MOT2 |
| MA0340.1 | MOT3 |
| MA1523.1 | MSANTD3 |
| MA0665.1 | MSC |
| MA1524.1 | MSGN1 |
| MA1433.1 | msn-1 |
| MA0341.1 | MSN2 |
| MA0342.1 | MSN4 |
| MA0666.1 | MSX1 |
| MA0708.1 | MSX2 |
| MA0709.1 | Msx3 |
| MA0863.1 | MTF1 |
| MA1108.2 | MXI1 |
| MA0100.3 | MYB |
| MA0054.1 | myb.Ph3 |
| MA1179.1 | MYB1 |
| MA1173.1 | MYB101 |
| MA1169.1 | MYB105 |
| MA1036.1 | MYB111 |
| MA1181.1 | MYB113 |
| MA1170.1 | MYB118 |
| MA1176.1 | MYB119 |
| MA1426.1 | MYB124 |
| MA0574.1 | MYB15 |
| MA1037.1 | MYB24 |
| MA1292.1 | MYB27 |
| MA1038.1 | MYB3 |
| MA1391.2 | MYB33 |
| MA1178.1 | MYB3R1 |
| MA1180.1 | MYB3R4 |
| MA1172.1 | MYB3R5 |
| MA1039.1 | MYB4 |
| MA1040.1 | MYB46 |
| MA1171.1 | MYB52 |
| MA1041.1 | MYB55 |
| MA1174.1 | MYB56 |
| MA1293.1 | MYB57 |
| MA1042.1 | MYB59 |
| MA1294.1 | MYB62 |
| MA1177.1 | MYB65 |
| MA1393.2 | MYB70 |
| MA1394.2 | MYB73 |
| MA1395.1 | MYB77 |
| MA1175.1 | MYB81 |
| MA1392.1 | MYB98 |
| MA0776.1 | MYBL1 |
| MA0777.1 | MYBL2 |
| MA0147.3 | MYC |
| MA0566.1 | MYC2 |
| MA0568.1 | MYC3 |
| MA0569.1 | MYC4 |
| MA0104.4 | MYCN |
| MA1641.1 | MYF5 |
| MA0667.1 | MYF6 |
| MA0499.2 | MYOD1 |
| MA0500.2 | MYOG |
| MA1168.1 | MYR2 |
| MA0056.2 | MZF1 |
| MA0057.1 | MZF1(var. 2) |
| MA1674.1 | NAC017 |
| MA0935.1 | NAC025 |
| MA1427.1 | NAC028 |
| MA1675.1 | NAC029 |
| MA1045.1 | NAC043 |
| MA0937.1 | NAC055 |
| MA0938.1 | NAC058 |
| MA1676.1 | NAC062 |
| MA1677.1 | NAC078 |
| MA0939.1 | NAC080 |
| MA1043.1 | NAC083 |
| MA1044.1 | NAC92 |
| MA0929.1 | NCU00019 |
| MA1434.1 | NCU02182 |
| MA0343.1 | NDT80 |
| MA1109.1 | NEUROD1 |
| MA0668.1 | NEUROD2 |
| MA0623.2 | NEUROG1 |
| MA0669.1 | NEUROG2 |
| MA1642.1 | NEUROG2(var. 2) |
| MA0606.1 | NFAT5 |
| MA0624.1 | NFATC1 |
| MA0152.1 | NFATC2 |
| MA0625.1 | NFATC3 |
| MA1525.1 | NFATC4 |
| MA0841.1 | NFE2 |
| MA0089.2 | NFE2L1 |
| MA0150.2 | Nfe2l2 |
| MA0670.1 | NFIA |
| MA1643.1 | NFIB |
| MA0161.2 | NFIC |
| MA0119.1 | NFIC::TLX1 |
| MA1527.1 | NFIC(var. 2) |
| MA0025.2 | NFIL3 |
| MA0671.1 | NFIX |
| MA1528.1 | NFIX(var. 2) |
| MA0105.4 | NFKB1 |
| MA0778.1 | NFKB2 |
| MA0060.3 | NFYA |
| MA0502.2 | NFYB |
| MA1644.1 | NFYC |
| MA0048.2 | NHLH1 |
| MA1529.1 | NHLH2 |
| MA0344.1 | NHP10 |
| MA0345.1 | NHP6A |
| MA0346.1 | NHP6B |
| MA1451.1 | nhr-6 |
| MA1435.1 | nit-4 |
| MA0196.1 | NK7.1 |
| MA1645.1 | NKX2-2 |
| MA0672.1 | NKX2-3 |
| MA0063.2 | NKX2-5 |
| MA0503.1 | Nkx2-5(var. 2) |
| MA0673.1 | NKX2-8 |
| MA0124.2 | Nkx3-1 |
| MA0122.3 | Nkx3-2 |
| MA0674.1 | NKX6-1 |
| MA0675.1 | NKX6-2 |
| MA1530.1 | NKX6-3 |
| MA0125.1 | Nobox |
| MA0710.1 | NOTO |
| MA0626.1 | Npas2 |
| MA1531.1 | NR1D1 |
| MA1532.1 | NR1D2 |
| MA0115.1 | NR1H2::RXRA |
| MA0494.1 | Nr1h3::Rxra |
| MA1110.1 | NR1H4 |
| MA1146.1 | NR1H4::RXRA |
| MA1533.1 | NR1I2 |
| MA1534.1 | NR1I3 |
| MA1535.1 | NR2C1 |
| MA0504.1 | NR2C2 |
| MA1536.1 | NR2C2(var. 2) |
| MA0676.1 | Nr2e1 |
| MA0164.1 | Nr2e3 |
| MA0017.2 | NR2F1 |
| MA1537.1 | NR2F1(var. 2) |
| MA1538.1 | NR2F1(var. 3) |
| MA1111.1 | NR2F2 |
| MA0677.1 | Nr2f6 |
| MA0728.1 | Nr2f6(var. 2) |
| MA1539.1 | NR2F6(var. 3) |
| MA0113.3 | NR3C1 |
| MA0727.1 | NR3C2 |
| MA1112.2 | NR4A1 |
| MA0160.1 | NR4A2 |
| MA1147.1 | NR4A2::RXRA |
| MA1540.1 | NR5A1 |
| MA0505.1 | Nr5a2 |
| MA1541.1 | NR6A1 |
| MA0506.1 | NRF1 |
| MA0347.1 | NRG1 |
| MA0842.2 | NRL |
| MA0421.1 | NSI1 |
| MA1678.1 | NTL8 |
| MA1046.1 | NTL9 |
| MA0197.2 | nub |
| MA1157.1 | NUC |
| MA1436.1 | nuc-1 |
| MA1417.1 | O2 |
| MA0348.1 | OAF1 |
| MA1278.1 | OBP1 |
| MA1274.1 | OBP3 |
| MA1280.1 | OBP4 |
| MA0234.1 | oc |
| MA0454.1 | odd |
| MA0198.1 | OdsH |
| MA1033.1 | OJ1058_F05.8 |
| MA1031.1 | OJ1581_H09.2 |
| MA0826.1 | OLIG1 |
| MA0678.1 | OLIG2 |
| MA0827.1 | OLIG3 |
| MA0235.1 | onecut |
| MA0679.2 | ONECUT1 |
| MA0756.1 | ONECUT2 |
| MA0757.1 | ONECUT3 |
| MA0456.1 | opa |
| MA0349.1 | OPI1 |
| MA0199.1 | Optix |
| MA1034.1 | 0s05g0497200 |
| MA1050.1 | Osl_08196 |
| MA1542.1 | OSR1 |
| MA1646.1 | OSR2 |
| MA1409.1 | OsRR22 |
| MA0236.1 | otp |
| MA0711.1 | OTX1 |
| MA0712.2 | OTX2 |
| MA0126.1 | ovo |
| MA1544.1 | OVOL1 |
| MA1545.1 | OVOL2 |
| MA1030.1 | P0510F09.23 |
| MA0924.1 | pal-1 |
| MA0237.2 | pan |
| MA0779.1 | PAX1 |
| MA0067.1 | Pax2 |
| MA0780.1 | PAX3 |
| MA1546.1 | PAX3(var. 2) |
| MA0068.2 | PAX4 |
| MA0014.3 | PAX5 |
| MA0069.1 | PAX6 |
| MA0680.1 | PAX7 |
| MA0781.1 | PAX9 |
| MA0238.1 | pb |
| MA0064.1 | PBF |
| MA0070.1 | PBX1 |
| MA1113.2 | PBX2 |
| MA1114.1 | PBX3 |
| MA1702.1 | Pdp1 |
| MA0352.1 | PDR1 |
| MA0353.1 | PDR3 |
| MA0354.1 | PDR8 |
| MA0132.2 | PDX1 |
| MA0127.1 | PEND |
| MA0546.1 | pha-4 |
| MA0355.1 | PHD1 |
| MA0457.1 | PHDP |
| MA1389.1 | PHL1 |
| MA1163.1 | PHL11 |
| MA1166.1 | PHL12 |
| MA1384.1 | PHL7 |
| MA1460.1 | pho |
| MA0356.1 | PHO2 |
| MA0357.1 | PHO4 |
| MA0713.1 | PHOX2A |
| MA0681.2 | PHOX2B |
| MA0987.1 | PHYPADRAFT_140773 |
| MA0988.1 | PHYPADRAFT_143875 |
| MA0989.1 | PHYPADRAFT_153324 |
| MA1007.1 | PHYPADRAFT_173530 |
| MA1008.1 | PHYPADRAFT_182268 |
| MA1023.1 | PHYPADRAFT_28324 |
| MA1022.1 | PHYPADRAFT_38837 |
| MA1021.1 | PHYPADRAFT_48267 |
| MA1010.1 | PHYPADRAFT_64121 |
| MA1011.1 | PHYPADRAFT_72483 |
| MA0559.1 | PI |
| MA0552.1 | PIF1 |
| MA0560.1 | PIF3 |
| MA0561.1 | PIF4 |
| MA0562.1 | PIF5 |
| MA1364.1 | PIF7 |
| MA0682.2 | PITX1 |
| MA1547.1 | PITX2 |
| MA0714.1 | PITX3 |
| MA0782.2 | PKNOX1 |
| MA0783.1 | PKNOX2 |
| MA0163.1 | PLAG1 |
| MA1615.1 | Plagl1 |
| MA1548.1 | PLAGL2 |
| MA1377.1 | PLT1 |
| MA1378.1 | PLT3 |
| MA0536.1 | pnr |
| MA0955.1 | PQPTR_0002s00440g |
| MA0784.1 | POU1F1 |
| MA0785.1 | POU2F1 |
| MA0507.1 | POU2F2 |
| MA0627.2 | POU2F3 |
| MA0786.1 | POU3F1 |
| MA0787.1 | POU3F2 |
| MA0788.1 | POU3F3 |
| MA0789.1 | POU3F4 |
| MA0790.1 | POU4F1 |
| MA0683.1 | POU4F2 |
| MA0791.1 | POU4F3 |
| MA1115.1 | POU5F1 |
| MA0142.1 | Pou5f1::Sox2 |
| MA0792.1 | POU5F1B |
| MA0628.1 | POU6F1 |
| MA1549.1 | POU6F1(var. 2) |
| MA0793.1 | POU6F2 |
| MA1148.1 | PPARA::RXRA |
| MA1550.1 | PPARD |
| MA0066.1 | PPARG |
| MA0065.2 | Pparg::Rxra |
| MA0200.1 | Pph13 |
| MA1703.1 | pqm-1 |
| MA0239.1 | prd |
| MA0508.3 | PRDM1 |
| MA1616.1 | Prdm15 |
| MA1647.1 | PRDM4 |
| MA0715.1 | PROP1 |
| MA0794.1 | PROX1 |
| MA0716.1 | PRRX1 |
| MA0075.3 | PRRX2 |
| MA1282.1 | PTF1 |
| MA1618.1 | Ptf1a |
| MA1619.1 | Ptf1a(var. 2) |
| MA1620.1 | Ptf1a(var. 3) |
| MA0201.1 | Ptx1 |
| MA1252.1 | PUCHI |
| MA0358.1 | PUT3 |
| MA1416.1 | RAMOSA1 |
| MA0359.1 | RAP1 |
| MA1679.1 | RAP2-1 |
| MA0980.2 | RAP2-10 |
| MA1051.1 | RAP2-3 |
| MA1052.1 | RAP2-6 |
| MA1216.1 | RAP21 |
| MA1249.1 | RAP210 |
| MA1266.1 | RAP211 |
| MA1256.1 | RAP212 |
| MA1221.1 | RAP26 |
| MA0729.1 | RARA |
| MA0159.1 | RARA::RXRA |
| MA1149.1 | RARA::RXRG |
| MA0730.1 | RARA(var. 2) |
| MA0857.1 | Rarb |
| MA0858.1 | Rarb(var. 2) |
| MA1552.1 | RARB(var. 3) |
| MA0859.1 | Rarg |
| MA0860.1 | Rarg(var. 2) |
| MA1553.1 | RARG(var. 3) |
| MA0582.1 | RAV1 |
| MA0583.1 | RAV1(var. 2) |
| MA0718.1 | RAX |
| MA0717.1 | RAX2 |
| MA0576.1 | RAX3 |
| MA1116.1 | RBPJ |
| MA1621.1 | Rbpj1 |
| MA0360.1 | RDR1 |
| MA0361.1 | RDS1 |
| MA0362.1 | RDS2 |
| MA0363.1 | REB1 |
| MA1415.1 | REF6 |
| MA0364.1 | REI1 |
| MA0101.1 | REL |
| MA0107.1 | RELA |
| MA1117.1 | RELB |
| MA0240.1 | repo |
| MA0138.2 | REST |
| MA0365.1 | RFX1 |
| MA0509.2 | RFX1 |
| MA0600.2 | RFX2 |
| MA0798.2 | RFX3 |
| MA0799.1 | RFX4 |
| MA0510.2 | RFX5 |
| MA1554.1 | RFX7 |
| MA0366.1 | RGM1 |
| MA0367.1 | RGT1 |
| MA0629.1 | Rhox11 |
| MA0719.1 | RHOXF1 |
| MA0368.1 | RIM101 |
| MA0369.1 | RLM1 |
| MA0370.1 | RME1 |
| MA0241.1 | ro |
| MA0071.1 | RORA |
| MA0072.1 | RORA(var. 2) |
| MA1150.1 | RORB |
| MA1151.1 | RORC |
| MA0371.1 | ROX1 |
| MA0372.1 | RPH1 |
| MA0373.1 | RPN4 |
| MA0073.1 | RREB1 |
| MA0374.1 | RSC3 |
| MA0375.1 | RSC30 |
| MA1431.1 | rst2 |
| MA0376.1 | RTG3 |
| MA0002.2 | RUNX1 |
| MA0511.2 | RUNX2 |
| MA0684.2 | RUNX3 |
| MA1184.1 | RVE1 |
| MA1190.1 | RVE5 |
| MA1183.1 | RVE6 |
| MA1680.1 | RVE7 |
| MA0202.1 | Rx |
| MA0512.2 | Rxra |
| MA0074.1 | RXRA::VDR |
| MA0855.1 | RXRB |
| MA1555.1 | RXRB(var. 2) |
| MA0856.1 | RXRG |
| MA1556.1 | RXRG(var. 2) |
| MA0193.1 | schlank |
| MA0203.1 | Scr |
| MA0743.2 | SCRT1 |
| MA0744.2 | SCRT2 |
| MA0243.1 | sd |
| MA0584.1 | 1-Sep |
| MA0563.1 | 3-Sep |
| MA0377.1 | SFL1 |
| MA0378.1 | SFP1 |
| MA1159.1 | SGR5 |
| MA0630.1 | SHOX |
| MA0720.1 | Shox2 |
| MA0380.1 | SIP4 |
| MA1118.1 | SIX1 |
| MA1119.1 | SIX2 |
| MA0631.1 | Six3 |
| MA0204.1 | Six4 |
| MA1405.1 | SIZF2 |
| MA0547.1 | skn-1 |
| MA0381.1 | SKN7 |
| MA0382.1 | SKO1 |
| MA0244.1 | slbo |
| MA0245.1 | slou |
| MA0458.1 | slp1 |
| MA0925.1 | sma-4 |
| MA1622.1 | Smad2::Smad3 |
| MA0513.1 | SMAD2::SMAD3::SMAD4 |
| MA0795.1 | SMAD3 |
| MA1153.1 | Smad4 |
| MA1557.1 | SMAD5 |
| MA0383.1 | SMP1 |
| MA0553.1 | SMZ |
| MA0086.2 | sna |
| MA1558.1 | SNAI1 |
| MA0745.2 | SNAI2 |
| MA1559.1 | SNAI3 |
| MA0544.1 | snpc-4 |
| MA0384.1 | SNT2 |
| MA0246.1 | so |
| MA0554.1 | SOC1 |
| MA1560.1 | SOHLH2 |
| MA0385.1 | SOK2 |
| MA1379.1 | SOL1 |
| MA0870.1 | Sox1 |
| MA0442.2 | SOX10 |
| MA0869.1 | Sox11 |
| MA1561.1 | SOX12 |
| MA1120.1 | SOX13 |
| MA1562.1 | SOX14 |
| MA1152.1 | SOX15 |
| MA0078.1 | Sox17 |
| MA1563.1 | SOX18 |
| MA0143.4 | SOX2 |
| MA0866.1 | SOX21 |
| MA0514.1 | Sox3 |
| MA0867.2 | SOX4 |
| MA0087.1 | Sox5 |
| MA0515.1 | Sox6 |
| MA0868.2 | SOX8 |
| MA0077.1 | SOX9 |
| MA0079.4 | SP1 |
| MA0516.2 | SP2 |
| MA0746.2 | SP3 |
| MA0685.1 | SP4 |
| MA0747.1 | SP8 |
| MA1564.1 | SP9 |
| MA0686.1 | SPDEF |
| MA0080.5 | SPI1 |
| MA0081.2 | SPIB |
| MA0687.1 | SPIC |
| MA1055.1 | SPL1 |
| MA1056.1 | SPL11 |
| MA1057.1 | SPL12 |
| MA1321.1 | SPL13 |
| MA0586.2 | SPL14 |
| MA1320.1 | SPL15 |
| MA0577.2 | SPL3 |
| MA1058.1 | SPL4 |
| MA1059.2 | SPL5 |
| MA1060.1 | SPL7 |
| MA0578.1 | SPL8 |
| MA1322.1 | SPL9 |
| MA1061.1 | SPT |
| MA0386.1 | SPT15 |
| MA0387.1 | SPT2 |
| MA0388.1 | SPT23 |
| MA0111.1 | Spz1 |
| MA0082.1 | squamosa |
| MA0389.1 | SRD1 |
| MA0595.1 | SREBF1 |
| MA0829.2 | SREBF1(var. 2) |
| MA0596.1 | SREBF2 |
| MA0828.1 | SREBF2(var. 2) |
| MA0083.3 | SRF |
| MA1681.1 | SRM1 |
| MA0084.1 | SRY |
| MA0137.3 | STAT1 |
| MA0517.1 | STAT1::STAT2 |
| MA1623.1 | Stat2 |
| MA0144.2 | STAT3 |
| MA0518.1 | Stat4 |
| MA1624.1 | Stat5a |
| MA0519.1 | Stat5a::Stat5b |
| MA1625.1 | Stat5b |
| MA0520.1 | Stat6 |
| MA0532.1 | Stat92E |
| MA0390.1 | STB3 |
| MA0391.1 | STB4 |
| MA0392.1 | STB5 |
| MA1410.1 | StBRC1 |
| MA0393.1 | STE12 |
| MA0394.1 | STP1 |
| MA0395.1 | STP2 |
| MA0396.1 | STP3 |
| MA0397.1 | STP4 |
| MA1372.1 | STZ |
| MA0085.1 | Su(H) |
| MA0533.1 | su(Hw) |
| MA0398.1 | SUM1 |
| MA0399.1 | SUT1 |
| MA0400.1 | SUT2 |
| MA1461.1 | SV |
| MA0555.1 | SVP |
| MA0401.1 | SWI4 |
| MA0402.1 | SWI5 |
| MA0936.1 | T11\|18.17 |
| MA0091.1 | TAL1::TCF3 |
| MA1430.1 | TB1 |
| MA0403.1 | TBF1 |
| MA0108.2 | TBP |
| MA1355.1 | TBP3 |
| MA0802.1 | TBR1 |
| MA0404.1 | TBS1 |
| MA0805.1 | TBX1 |
| MA0803.1 | TBX15 |
| MA1565.1 | TBX18 |
| MA0804.1 | TBX19 |
| MA0688.1 | TBX2 |
| MA0689.1 | TBX20 |
| MA0690.1 | TBX21 |
| MA1566.1 | TBX3 |
| MA0806.1 | TBX4 |
| MA0807.1 | TBX5 |
| MA1567.1 | TBX6 |
| MA0009.2 | TBXT |
| MA0521.1 | Tcf12 |
| MA1648.1 | TCF12(var. 2) |
| MA0832.1 | Tcf21 |
| MA1568.1 | TCF21(var. 2) |
| MA0522.3 | TCF3 |
| MA0830.2 | TCF4 |
| MA0769.2 | TCF7 |
| MA1421.1 | TCF7L1 |
| MA0523.1 | TCF7L2 |
| MA0632.2 | TCFL5 |
| MA1284.1 | TCP1 |
| MA1283.1 | TCP14 |
| MA1062.2 | TCP15 |
| MA0587.1 | TCP16 |
| MA1290.1 | TCP17 |
| MA1063.1 | TCP19 |
| MA1064.1 | TCP2 |
| MA1065.2 | TCP20 |
| MA1287.1 | TCP21 |
| MA1066.1 | TCP23 |
| MA1286.1 | TCP24 |
| MA1289.1 | TCP3 |
| MA1035.1 | TCP4 |
| MA1067.1 | TCP5 |
| MA1291.1 | TCP7 |
| MA1428.1 | TCP8 |
| MA1162.1 | TCX2 |
| MA1682.1 | TCX3 |
| MA0431.1 | TDA9 |
| MA0405.1 | TEA1 |
| MA0090.3 | TEAD1 |
| MA1121.1 | TEAD2 |
| MA0808.1 | TEAD3 |
| MA0809.2 | TEAD4 |
| MA0406.1 | TEC1 |
| MA0843.1 | TEF |
| MA0003.4 | TFAP2A |
| MA0810.1 | TFAP2A(var. 2) |
| MA0872.1 | TFAP2A(var. 3) |
| MA0811.1 | TFAP2B |
| MA0812.1 | TFAP2B(var. 2) |
| MA0813.1 | TFAP2B(var. 3) |
| MA0524.2 | TFAP2C |
| MA0814.2 | TFAP2C(var. 2) |
| MA0815.1 | TFAP2C(var. 3) |
| MA1569.1 | TFAP2E |
| MA0691.1 | TFAP4 |
| MA1570.1 | TFAP4(var. 2) |
| MA0145.3 | TFCP2 |
| MA1122.1 | TFDP1 |
| MA0831.2 | TFE3 |
| MA0692.1 | TFEB |
| MA0871.2 | TFEC |
| MA0588.1 | TGA1 |
| MA1346.1 | TGA10 |
| MA0129.1 | TGA1A |
| MA1068.1 | TGA2 |
| MA1336.1 | TGA3 |
| MA1335.1 | TGA4 |
| MA1047.2 | TGA5 |
| MA1069.1 | TGA6 |
| MA1070.1 | TGA7 |
| MA1348.1 | TGA9 |
| MA0796.1 | TGIF1 |
| MA0797.1 | TGIF2 |
| MA1571.1 | TGIF2LX |
| MA1572.1 | TGIF2LY |
| MA0597.1 | THAP1 |
| MA1573.1 | THAP11 |
| MA0407.1 | THI2 |
| MA1574.1 | THRB |
| MA1575.1 | THRB(var. 2) |
| MA1576.1 | THRB(var. 3) |
| MA0247.2 | tin |
| MA1220.1 | TINY |
| MA0459.1 | tll |
| MA1577.1 | TLX2 |
| MA0350.1 | TOD6 |
| MA0408.1 | TOS8 |
| MA0106.3 | TP53 |
| MA0525.2 | TP63 |
| MA0861.1 | TP73 |
| MA0205.2 | Trl |
| MA1352.1 | TRP1 |
| MA1356.1 | TRP2 |
| MA1411.1 | TSAR1 |
| MA1412.1 | TSAR2 |
| MA1161.1 | TSO1 |
| MA0460.1 | ttk |
| MA0248.1 | tup |
| MA0249.2 | twi |
| MA1123.2 | TWIST1 |
| MA0633.1 | Twist2 |
| MA0409.1 | TYE7 |
| MA0094.2 | Ubx |
| MA0410.1 | UGA3 |
| MA1413.1 | UIF1 |
| MA0412.1 | UME6 |
| MA1443.1 | unc-30 |
| MA0250.1 | unc-4 |
| MA0918.1 | unc-62 |
| MA0926.1 | unc-86 |
| MA0721.1 | UNCX |
| MA1074.1 | UNE10 |
| MA0251.1 | unpg |
| MA0411.1 | UPC2 |
| MA0422.1 | URC2 |
| MA0093.3 | USF1 |
| MA0526.3 | UMA0256.1 |
| MA0257.1 | zen2 |
| MA0928.1 | zfh-2 |
| MA1651.1 | ZFP42 |
| MA1583.1 | ZFP57 |
| MA0146.2 | Zfx |
| MA1329.2 | ZHD1 |
| MA1213.1 | ZHD3 |
| MA1326.1 | ZHD5 |
| MA1330.1 | ZHD6 |
| MA0696.1 | ZIC1 |
| MA1628.1 | Zic1::Zic2 |
| MA1629.1 | Zic2 |
| MA0697.1 | ZIC3 |
| MA0751.1 | ZIC4 |
| MA1584.1 | ZIC5 |
| MA1704.1 | zip-8 |
| MA1585.1 | ZKSCAN1 |
| MA1652.1 | ZKSCAN5 |
| MA0441.1 | ZMS1 |
| MA1587.1 | ZNF135 |
| MA1588.1 | ZNF136 |
| MA1589.1 | ZNF140 |
| MA0088.2 | ZNF143 |
| MA1653.1 | ZNF148 |
| MA1654.1 | ZNF16 |
| MA1124.1 | ZNF24 |
| MA0528.2 | ZNF263 |
| MA1592.1 | ZNF274 |
| MA1630.1 | Znf281 |
| MA1154.1 | ZNF282 |
| MA1593.1 | ZNF317 |
| MA1655.1 | ZNF341 |
| MA0130.1 | ZNF354C |
| MA1594.1 | ZNF382 |
| MA1125.1 | ZNF384 |
| MA0752.1 | ZNF410 |
| MA0116.1 | Znf423 |
| MA1656.1 | ZNF449 |
| MA1596.1 | ZNF460 |
| MA1597.1 | ZNF528 |
| MA1657.1 | ZNF652 |
| MA1599.1 | ZNF682 |
| 1MA1600.1 | ZNF684 |
| MA0753.2 | ZNF740 |
| MA1601.1 | ZNF75D |
| MA1602.1 | ZSCAN29 |
| MA1155.1 | ZSCAN4 |
| KY48 | |
| MA1317.1 | WRKY50 |
| MA1305.1 | WRKY55 |
| MA1089.1 | WRKY57 |
| MA1304.1 | WRKY59 |
| MA1300.1 | WRKY6 |
| MA1090.1 | WRKY60 |
| MA1091.1 | WRKY62 |
| MA1092.1 | WRKY63 |
| MA1302.1 | WRKY65 |
| MA1313.1 | WRKY7 |
| MA1308.1 | WRKY70 |
| MA1316.1 | WRKY71 |
| MA1093.1 | WRKY75 |
| MA1094.1 | WRKY8 |
| MA1627.1 | Wt1 |
| MA0414.1 | XBP1 |
| MA0844.1 | XBP1 |
| MA0415.1 | YAP1 |
| MA0416.1 | YAP3 |
| MA0417.1 | YAP5 |
| MA0418.1 | YAP6 |
| MA0419.1 | YAP7 |
| MA0423.1 | YER130C |
| MA0424.1 | YER184C |
| MA0425.1 | YGR067C |
| MA0426.1 | YHPI |
| MA0428.1 | YKL222C |
| MA0429.1 | YLL054C |
| MA0430.1 | YLR278C |
| MA0432.1 | YNR063W |
| MA0433.1 | YOX1 |
| MA0434.1 | YPR013C |
| MA0435.1 | YPR015C |
| MA0436.1 | YPR022C |
| MA0437.1 | YPR196W |
| MA0438.1 | YRM1 |
| MA0439.1 | YRR1 |
| MA0095.2 | YYl |
| MA0748.2 | YY2 |
| MA0255.1 | z |
| MA0440.1 | ZAP1 |
| MA0589.1 | ZAP1 |
| MA0749.1 | ZBED1 |
| MA1649.1 | ZBTB12 |
| MA1650.1 | ZBTB14 |
| MA0698.1 | ZBTB18 |
| MA1579.1 | ZBTB26 |
| MA1580.1 | ZBTB32 |
| MA0527.1 | ZBTB33 |
| MA1581.1 | ZBTB6 |
| MA0750.2 | ZBTB7A |
| MA0694.1 | ZBTB7B |
| MA0695.1 | ZBTB7C |
| MA0103.3 | ZEB1 |
| MA0927.1 | vab-7 |
| MA0722.1 | VAX1 |
| MA0723.1 | VAX2 |
| MA0693.2 | VDR |
| MA0724.1 | VENTX |
| MA1578.1 | VEZF1 |
| MA1462.1 | vfl |
| MA0252.1 | vis |
| MA0253.1 | vnd |
| MA0181.1 | Vsx1 |
| MA0725.1 | VSX1 |
| MA0180.1 | Vsx2 |
| MA0726.1 | VSX2 |
| MA0254.1 | vvl |
| MA1437.1 | wc-1 |
| MA1306.1 | WRKY11 |
| MA1075.1 | WRKY12 |
| MA1314.1 | WRKY14 |
| MA1076.2 | WRKY15 |
| MA1299.1 | WRKY17 |
| MA1077.1 | WRKY18 |
| MA1078.1 | WRKY2 |
| MA1295.1 | WRKY20 |
| MA1079.1 | WRKY21 |
| MA1303.1 | WRKY22 |
| MA1080.1 | WRKY23 |
| MA1315.1 | WRKY24 |
| MA1081.1 | WRKY25 |
| MA1297.1 | WRKY26 |
| MA1318.1 | WRKY27 |
| MA1311.2 | WRKY28 |
| MA1298.1 | WRKY29 |
| MA1309.1 | WRKY3 |
| MA1083.1 | WRKY30 |
| MA1307.1 | WRKY31 |
| MA1301.1 | WRKY33 |
| MA1084.1 | WRKY38 |
| MA1085.2 | WRKY40 |
| MA1310.1 | WRKY42 |
| MA1086.1 | WRKY43 |
| MA1087.1 | WRKY45 |
| MA1296.1 | WRKY46 |
| MA1312.1 | WRKY47 |
| MA1088.1 | WRKY48 |
| MA1317.1 | WRKY50 |
| MA1305.1 | WRKY55 |
| MA1089.1 | WRKY57 |
| MA1304.1 | WRKY59 |
| MA1300.1 | WRKY6 |
| MA1090.1 | WRKY60 |
| MA1091.1 | WRKY62 |
| MA1092.1 | WRKY63 |
| MA1302.1 | WRKY65 |
| MA1313.1 | WRKY7 |
| MA1308.1 | WRKY70 |
| MA1316.1 | WRKY71 |
| MA1093.1 | WRKY75 |
| MA1094.1 | WRKY8 |
| MA1627.1 | Wt1 |
| MA0414.1 | XBP1 |
| MA0844.1 | XBP1 |
| MA0415.1 | YAP1 |
| MA0416.1 | YAP3 |
| MA0417.1 | YAP5 |
| MA0418.1 | YAP6 |
| MA0419.1 | YAP7 |
| MA0423.1 | YER130C |
| MA0424.1 | YER184C |
| MA0425.1 | YGR067C |
| MA0426.1 | YHP1 |
| MA0428.1 | YKL222C |
| MA0429.1 | YLL054C |
| MA0430.1 | YLR278C |
| MA0432.1 | YNR063W |
| MA0433.1 | YOX1 |
| MA0434.1 | YPR013C |
| MA0435.1 | YPR015C |
| MA0436.1 | YPR022C |
| MA0437.1 | YPR196W |
| MA0438.1 | YRM1 |
| MA0439.1 | YRR1 |
| MA0095.2 | YYl |
| MA0748.2 | YY2 |
| MA0255.1 | z |
| MA0440.1 | ZAP1 |
| MA0589.1 | ZAP1 |
| MA0749.1 | ZBED1 |
| MA1649.1 | ZBTB12 |
| MA1650.1 | ZBTB14 |
| MA0698.1 | ZBTB18 |
| MA1579.1 | ZBTB26 |
| MA1580.1 | ZBTB32 |
| MA0527.1 | ZBTB33 |
| MA1581.1 | ZBTB6 |
| MA0750.2 | ZBTB7A |
| MA0694.1 | ZBTB7B |
| MA0695.1 | ZBTB7C |
| MA0103.3 | ZEB1 |
| MA0256.1 | zen |
| MA0257.1 | zen2 |
| MA0928.1 | zfh-2 |
| MA1651.1 | ZFP42 |
| MA1583.1 | ZFP57 |
| MA0146.2 | Zfx |
| MA1329.2 | ZHD1 |
| MA1213.1 | ZHD3 |
| MA1326.1 | ZHD5 |
| MA1330.1 | ZHD6 |
| MA0696.1 | ZIC1 |
| MA1628.1 | Zic1::Zic2 |
| MA1629.1 | Zic2 |
| MA0697.1 | ZIC3 |
| MA0751.1 | ZIC4 |
| MA1584.1 | ZIC5 |
| MA1704.1 | zip-8 |
| MA1585.1 | ZKSCAN1 |
| MA1652.1 | ZKSCAN5 |
| MA0441.1 | ZMS1 |
| MA1587.1 | ZNF135 |
| MA1588.1 | ZNF136 |
| MA1589.1 | ZNF140 |
| MA0088.2 | ZNF143 |
| MA1653.1 | ZNF148 |
| MA1654.1 | ZNF16 |
| MA1124.1 | ZNF24 |
| MA0528.2 | ZNF263 |
| MA1592.1 | ZNF274 |
| MA1630.1 | Znf281 |
| MA1154.1 | ZNF282 |
| MA1593.1 | ZNF317 |
| MA1655.1 | ZNF341 |
| MA0130.1 | ZNF354C |
| MA1594.1 | ZNF382 |
| MA1125.1 | ZNF384 |
| MA0752.1 | ZNF410 |
| MA0116.1 | Znf423 |
| MA1656.1 | ZNF449 |
| MA1596.1 | ZNF460 |
| MA1597.1 | ZNF528 |
| MA1657.1 | ZNF652 |
| MA1599.1 | ZNF682 |
| MA1600.1 | ZNF684 |
| MA0753.2 | ZNF740 |
| MA1601.1 | ZNF75D |
| MA1602.1 | ZSCAN29 |
| MA1155.1 | ZSCAN4 |

TABLE 3B
Examples of hormone responsive transcription factors and elements ²⁸

| Associating | | |
|---|---|---|
| Nuclear | | |
| Receptor | Ligand | Response Element |
| RORα,β,γ | retinoids | RORαRE |
| LXRα,β | oxysterols, including 22(R)- | LXRE |
| | hydroxycholesterol, 24(S)- | |
| | hydroxycholesterol, and 24(S),25- | |
| | epoxycholesterol | |
| FXR | Chenodeoxycholic acid and other | AGGTCA |
| | bile acids | |
| VDR | Vitamin D (1,25(OH)2D) | VDRE |
| | Lithocholic acid | |
| ERα,β | Estrogen | ERE-TATA, ERE |
| GR | cortisol and other glucocorticoids | decoy GRE, GRE, plfG |

The modification of endogenous transcription factor spacing by genetic editing is another viable strategy for gene activation. Transcription factor cooperativity^{30 31} is a well-known phenomenon where multiple transcription factors bound in proximity leads to greater overall binding stabilization and downstream regulatory effect. First, the analysis of endogenous transcription factor spacing for optimal regulatory potential can be determined based on primary DNA sequence in functional cis-regulatory elements or based on studies of transcription factor pair binding data in vitro (e.g., CAP-SELEX)¹⁶. Next, the mapping of endogenous transcription factor binding sites in cis-regulatory elements of a target gene of interest can be performed to determine sub-optimal endogenous transcription factor binding spacing³². The utilization of genetic modifiers that can introduce insertion or deletion edits (e.g., programmable nucleases, prime editors) can then be targeted to modify the endogenous transcription factor binding site distances to promote heritable target gene activation.

Selection of Target Genomic Regulatory Regions

The identification and selection of target genomic regulatory regions can be made based on identification or prediction (e.g., bioinformatic or empirical) of cis-regulatory elements (e.g., promoters, enhancers, insulators, silencers/repressors) that regulate a target gene or set of target genes of interest, untranslated regions (UTRs) of target gene transcripts that can modify transcript stability, or regions along the transcript that can modify translation initiation and/or elongation efficiency of the target gene transcript, or locations in the genome where expression of an non-coding RNA could lead to targeted gene repression.

In some embodiments, the target genomic regions are non-coding DNA sequences within 1 Mb or more of the target gene(s) of interest.

In some embodiments, the target genomic regions are promoters of the target gene(s) of interest, defined as proximal regions e.g., 1000 bp upstream and 500 bp downstream of the transcription start site (TSS).

In some embodiments, the target genomic regions are putative enhancer elements of the target gene(s) of interest defined by histone marks and/or chromatin accessibility features associated with functional enhancer elements (e.g., H3K4me1, H3K27ac)¹⁷.

In some embodiments, the target genomic regions are putative insulator elements of the target gene(s) of interest defined by histone marks and/or chromatin accessibility features associated with functional insulator elements^{17, 18}.

In some embodiments, the target genomic regions are putative silencer elements of the target gene(s) of interest defined by histone marks and/or chromatin accessibility features associated with functional silencer elements¹⁹.

In some embodiments, the target genomic regions are untranslated regions (UTRs) of target gene transcripts.

In some embodiments, the target genomic regions are intronic regions of target gene transcripts.

In some embodiments, the target genomic regions are coding sequences of target gene transcripts.

In some embodiments, the endogenous regulatory region of a gene (e.g., the promoter) is targeted to modify or enhance downstream transcription of translation machinery. This can be achieved e.g., by installing or modifying a TATA box (also known as Goldberg-Hogness box) in archae or eukaryotes or a Pribnow box in prokaryotes or by installing enhanced Kozak ((gcc)gccRccAUGG (SEQ ID NO: 478)) in eukaryotes, Shine-Dalgarno (AGGAGGU) in prokaryotes, start codon (AUG and CUG in mammalian cells, AUA and AUU in mitochondria, GUG and UUG in E. coli) or stop codon (UGA, UAG, UAA) sequences²⁰.

In some embodiments binding sites of non-coding RNAs (ncRNAs), such as microRNAs (miRNAs) or long non-coding RNAs (lncRNAs) are installed or modified to alter the binding of said ncRNAs to DNA or RNA with the result of altered gene expression, and/or RNA abundance, and/or protein expression.

Selection of Genetic Modifiers

The identification of genetic modifiers can be determined based on predictions (e.g., bioinformatic or empirical) of ability, efficiency and precision of a given modifier to introduce specific sequence motifs at a target region of interest (Table 1). The methods can include identifying genetic modifiers to introduce specific sequence motifs into target genomic regions with high predicted precision and efficiency to alter expression of a target gene or set of genes at the pre-transcriptional or post-transcriptional level, in the context of a cell type or set of cell types of interest.

In some embodiments, the methods can include using an algorithm that compares the target regulatory regions and regulatory motif sequences identified above and identifies candidate regulatory motif sequences. and compares the candidate regulatory motifs to the possible modifications that would be made by a set of genetic modifiers (e.g., to predict the modification(s) made by each of a set of genetic modifiers, to identify one or more genetic modifiers that can be used to modify the target regulatory region to introduce a functional regulatory motif. In some embodiments, the candidate regulatory motif sequences differ from the target gene regulatory region by less than a selected amount, e.g., by 1-50%, and a Base Editor or prime editor can be selected to make the changes. In some embodiments, there is no identity between the target regulatory regions and regulatory motif sequences, and a prime editor is selected for inserting a regulatory motif into the target regulatory region.

The identification of a genetic modifier (e.g., programmable nuclease, base editor, prime editor) can be performed through the unbiased saturating mutagenesis of regulatory regions (e.g., promoters, enhancers) associated with a target gene. Comprehensive genetic tiling of these regulatory elements and downstream readout of the editing effects on gene activation can be used to empirically identify combinations of genetic modifiers and target sites that yield varying levels of target gene activation. This strategy can be performed in a pooled library format where target gene activation is a selectable or sortable feature, allowing for the high-throughput identification of candidates for SHAPE.

In some embodiments, the sequence motif is introduced as a single motif or a repetitive sequence with multiple copies of the single motif that may or may not have linker sequences interspaced.

In some embodiments, the sequence motif is introduced as a combination of different sequence motifs with predicted additive or synergistic effects on target gene expression at the pre-transcriptional and/or post-transcriptional level, where the multiple sequence motifs may or may not have linker sequences interspaced.

In some embodiments, algorithms to predict sequence alleles following nuclease genome editing events are used to identify a programmable nuclease type (e.g., zinc finger nucleases, transcription activator-like effector nucleases, Cas9, CasX, Cas12) and target cleavage indice(s) within a DNA sequence of interest to produce an allele that resembles a sequence motif or modification of interest.

In some embodiments, algorithms focused on patterns of microhomology-mediated end joining (MMEJ) and/or non-homologous end joining (NHEJ) following nuclease-induced DBA double-strand breaks (DSBs) can be used for the prediction of alleles following genome editing by a programmable nuclease (e.g., zinc finger nucleases, transcription activator-like effector nucleases, Cas9, CasX, Cas12)²¹.

In some embodiments, the use of the predominant +1 insertion allele from Cas9 editing can be used to precisely install sequence motifs of interest²².

In some embodiments, algorithms to predict sequence alleles following base editing events can be used to identify the base editor type (e.g., ABEs, CBEs) and base editing window(s) within a DNA sequence of interest to produce an allele that resembles a sequence motif or modification of interest^{23,24 25}.

In some embodiments, algorithms to predict sequence alleles following base editing events can be used to identify the base editor type (e.g., ABEs, CBEs) and base editing window(s) within a DNA sequence of interest to produce an allele that modifies (e.g., strengthens, weakens) the regulatory potential of an existing sequence motif.

In some embodiments, algorithms to predict sequence alleles following prime editing events can be used to identify the prime editor type (e.g., SpCas9H840A-MMLV-RT), prime editing guide RNAs (pegRNAs), and nicking sgRNAs (ngRNAs) to install a specific sequence motif or modification that can affect the expression of the target gene at the pre-transcriptional or post-transcriptional level².

In some embodiments, prime editing can be used to install specific substitution edits within an endogenous sequence context to introduce specific sequence motifs of interest. In some embodiments, prime editing can be used to install specific insertion edits within an endogenous sequence context to introduce specific sequence motifs of interest.

In some embodiments, prime editing can be used to install specific insertion edits to modify endogenous sequence motifs within untranslated regions (UTRs) of target gene transcripts or insert sequences that contain RNA-stabilizing or de-stabilizing sequence motifs into untranslated regions (UTRs) of target gene transcripts to affect the abundance and/or expression of the target gene products (e.g., RNA, protein).

In some embodiments, prime editing can be used to install specific deletion edits within an endogenous sequence context to introduce specific sequence motifs of interest.

In some embodiments, prime editing can be used to install specific combination edits (e.g., substitution, insertion, and/or deletion edits) within an endogenous sequence context to introduce specific sequence motifs of interest

In some embodiments, the design of pegRNAs and ngRNAs for prime editing will introduce silent mutations into the protospacer adjacent motif (PAM).

In some embodiments, the design of ngRNAs for prime editing will preferentially target the genome following the editing event (known as PE3b).

In some embodiments, the introduction of a transcription factor (TF) binding site in promoter or enhancer sequences or any other DNA sequence that might affect transient or heritable gene activation can be induced using a base editor (BE). In this case an endogenous sequence would be altered by BEs to allow TF binding by increasing homology of the endogenous sequence to a known TF binding site, thereby enabling a SHAPE event (BE-SHAPE).

In some embodiments, the unbiased saturation mutagenesis across genomic sequences (e.g., promoter, enhancers, untranslated regions) can be performed to empirically determine the strategy for SHAPE.

In some embodiments, multiplex genetic editing can be utilized to induce more robust modification of target gene expression at the pre-transcriptional and/or post-transcriptional level.

In some embodiments, multiplex genetic editing can be utilized to modify multiple target gene expressions in parallel at the pre-transcriptional and/or post-transcriptional level.

In some embodiments, the installation of sequence motifs that act as response elements (Table 3C) can be used to perform inducible modification of gene expression (e.g., activation, repression)following the introduction of an exogenous small molecule, hormone, or drug.

In some embodiments, the installation of cell-type-specific sequence motifs can be used to achieve cell-type-specific modification of the expression of a target gene or set of target genes.

In some embodiments, the use of homology directed repair (HDR) through programmable nucleases and a donor DNA (e.g., ssODN, dsODN) can be used to introduce sequence motifs of interest into a target site²³.

In some embodiments, the use of NHEJ-mediated DNA sequence integration through programmable nucleases and double-stranded oligodeoxynucleotides (dsODN) or circular DNA donors (e.g., plasmids, minicircles) can be used to introduce sequence motifs of interest into a target site^{26 27}.

In some embodiments, a DNA sequence alteration described herein can be performed using CRISPR-guided DNA base editors such as the cytosine base editor (CBE) that allows for the introduction of C-to-T and G-to-A modifications, the adenine base editor (ABE) that allows for the introduction of A-to-G and T-to-C modifications, the cytosine-to-guanine transversion base editor (CGBE) that allows for the introduction of C-to-G and G-to-C modifications, as well as the synchronous programming of adenine and cytosine editor (SPACE) that allows for the simultaneous introduction of A-to-G (T-to-C on opposite strand) and C-to-T (G-to-A on opposite strand) modifications within the same editing window at the ssDNA bubble generated by RNA-guided fusion proteins.

While CBEs and CGBEs are comprised of a cytidine deaminase (e.g., pmCDA1, hAPOBEC3A, hAID, hAPOBEC3G or rAPOBEC1) as well as a CRISPR Cas RGN or a variant thereof, ABEs contain an adenosine deaminase (e.g., E. coli TadA or variants thereof as momoners or dimers) and a CRISPR Cas protein, and SPACE contains both adenine (e.g., E. coli TadA) and cytosine deaminases (e.g., pmCDA1) as well as CRISPR-Cas proteins (e.g., S. pyogenes Cas9).

In one aspect, the present invention relates to the use of an ABE or SPACE comprising: an adenosine deaminase, e.g., a wild type and/or engineered adenosine deaminase (e.g., ABEs 0.1, 0.2, 1.1, 1.2, 2.1, 2.2, 2.3, 2.4, 2.5, 2.6, 2.7, 2.8, 2.9, 2.10, 2.11, 2.12, 3.1, 3.2, 3.3, 3.4, 3.5, 3.6, 3.7, 3.8, 4.1, 4.2, 4.3, 5.1, 5.2, 5.3, 5.4, 5.5, 5.6, 5.7, 5.8, 5.9, 5.10, 5.11, 5.12, 5.13, 5.14, 6.1, 6.2, 6.3, 6.4, 6.5, 6.6, 7.1, 7.2, 7.3, 7.4, 7.5, 7.6, 7.7, 7.8, 7.9, 7.10, or ABEmax), E. coli TadA monomer, or variations of homo- or heterodimers thereof, bearing one or more mutations in either or both monomers (e.g., the TadA mutant used in miniABEmax-V82G, miniABEmax-K20A/R21A, miniABEmax-V106W, that decrease RNA editing activity while preserving DNA editing activity; a cytidine deaminase (e.g., pmCDA1, rat APOBEC1, human APOBEC3A, or human AID) or variations thereof with reduced RNA off-target editing, one or multiple uracil-n-clycosalyse inhibitors (UGIs); and a programmable DNA binding domain (e.g., Cas9-D10A); and optionally further comprising one or more nuclear localization sequences (e.g., SV40 large T antigen NLS (PKKKRRV, SEQ ID NO: 1), bipartite NLS (KRTADGSEFESPKKKRKV, SEQ ID NO. 2), or nucleoplasmin NLS (KRPAATKKAGQAKKKK, SEQ ID NO. 3). Other NLSs are known in the art; see, e.g., Cokol et al., EMBO Rep. 2000 Nov. 15; 1(5): 411-415; Freitas and Cunha, Curr Genomics. 2009 December; 10(8): 550-557.

In some embodiments, the dual-deaminase BE SPACE would be used and it would include a heterodimeric combined N-terminal adenosine and cytidine deaminase fusion (e.g., pmCDA1 or rAPOBEC1 or hA3A or AID fused to TadA monomers or dimers with a linker) or a heterodimeric combined C-terminal adenosine and cytidine deaminase fusion (e.g., pmCDA1 or rAPOBEC1 or hA3A or AID fused to TadA monomers or dimers with a linker). In both N- and C-terminal positions of these “hybrid fusion deaminase designs” the deaminases can be fused in either of these two orders: NH2-cytidine deaminase-linker-adenosine deaminase or NH2-adenosine deaminase-linker-cytidine deaminase.

In some embodiments, the programmable DNA binding domain is selected from the group consisting of engineered C2H2 zinc-fingers, transcription activator effector-like effectors (TALEs), and Clustered Regularly Interspaced Short Palindromic Repeats (CRISPR) Cas RNA-guided nucleases (RGNs) and variants thereof (e.g., Tables F L and MG).

In some embodiments, the CRISPR RGN is an ssDNA nickase or is catalytically inactive, e.g., a Cas9, CasX or Cas12a that has ssDNA nickase activity or is catalytically inactive.

In one aspect, the present invention relates to a base editing system comprising: (i) ABE, CBE, CGBE, or SPACE, wherein the programmable DNA binding domain is a CRISPR Cas RGN or a variant thereof; and (ii) at least one guide RNA compatible with the base editor that directs the base editor to a target sequence which is can then be deaminated in order to generate e.g., a TF binding site or any other modification that allows for the transcriptional activation of a targeted gene.

In one aspect, the present invention relates to an isolated nucleic acid encoding any of the CBEs, ABEs, CGBEs, and SPACE or other base editing systems described herein to induce a BE-SHAPE event.

In one aspect, the present invention relates to a vector comprising an isolated nucleic acid described herein.

In one aspect, the present invention relates to an isolated host cell, preferably a mammalian host cell, comprising any of the nucleic acids described herein.

In some embodiments, the isolated host cell described herein expresses a base editor and a gRNA for DNA modification to induce a BE-SHAPE event described herein.

In one aspect, the present invention relates to a method of deaminating a selected adenine and/or cytosine in a nucleic acid, the method comprising contacting the nucleic acid with SPACE, a base editing system, an isolated nucleic acid, a vector, or an isolated host cell described herein.

In one aspect, the present invention relates to a composition comprising a purified CBE, ABE, SPACE, or CGBE, a base editing system, an isolated nucleic acid, a vector, or an isolated host cell described herein.

In some embodiments, the composition includes one or more ribonucleoprotein (RNP) complexes.

In some embodiments, CBE, ABE, CGBE, or SPACE that are being used for BE-SHAPE comprise one or more uracil-N-glycosylase inhibitors (UGIs). In some embodiments, the base editors comprise a linker between the adenosine deaminase and the programmable DNA binding domain as well as the cytidine deaminase and the DNA binding domain, or both in the case of SPACE. In some embodiments the TadA domain can be monomeric, homodimeric or heterodimeric and contain all combinations of wild type (WT) E. coli TadA, or mutant variants of TadA.

In some embodiments one or two deaminase domains can be located at the C-terminus (e.g., pmCDA1) and N-terminus (TadA) or vice versa or they can both be located at the C- or N-terminus.

In some embodiments, the programmable DNA binding domain is selected from the group consisting of engineered C2H2 zinc-fingers, transcription activator effector-like effectors (TALEs), and Clustered Regularly Interspaced Short Palindromic Repeats (CRISPR)-Cas RNA-guided nucleases (RGNs) and variants thereof.

In some embodiments, the CRISPR-Cas RGN is an ssDNA nickase or is catalytically inactive, e.g., a Cas9 or Cas12a that is catalytically inactive or has ssDNA nickase activity (Table 4A).

In some embodiments, the methods include the use of base editing systems comprising (i) the adenine base editors described herein, wherein the programmable DNA binding domain is a CRISPR Cas RGN or a variant thereof (Table 4B); and (ii) at least one guide RNA compatible with the base editor that directs the base editor to a target sequence.

In some embodiments, the methods include the use of nucleic acids encoding ABE, CBE, CGBE or SPACE; vectors comprising the isolated nucleic acids; and isolated host cells, preferably mammalian host cells, comprising the nucleic acids. In some embodiments, the isolated host cell expresses an adenine base editor.

In some embodiments, the methods include the use of methods for deaminating a selected adenine in a nucleic acid, the method comprising contacting the nucleic acid with an adenine base editor or base editing system as described herein.

In some embodiments, the methods include the use of compositions comprising a purified ABE, CBE, CGBE or SPACE or base editing system as described herein. In some embodiments, the composition comprises one or more ribonucleoprotein (RNP) complexes.

In some embodiments, the guide RNAs that are used to target BEs for BE-SHAPE can be truncated by reducing spacer length to <20 nucleotides, e.g., to 17-18 nucleotides, which has been shown to enhance specificity of CRISPR-Cas nucleases (Fu & Sander et al, Nature Biotechnology 2014, 32(3):279-284) and may also affect base editing windows (Kim et al, Nature Biotechnology 2017, 35(4): 371-376).

In some embodiments, multiplex base editing with CBE, ABE, CGBE, and SPACE can be used to enhance the efficiency of BE-SHAPE. In this aspect, one or more gRNAs can be guiding one or more base editors (e.g., CBE and ABE, or ABE and CGBE, or SPACE and CGBE) to one or more genomic target sites to install multiple TF binding sites (or other sequence changes that drive transcriptional activation) at once in one cell or a population of cells, or a tissue, both in vivo or ex vivo.

In some embodiments, anti-CRISPR tools (Bondy-Denomy et al, Nature 2013, 493(7432):429-32 and Nature 2015, 526(7571):136-9; Pawluk et al, Cell 2016, 167(7):1829-1838.e9.) can be used to control the efficiency of CRISPR-based SHAPE platforms by altering the capacity of DNA binding and/or cleavage of the CRISPR-Cas protein when used as a nuclease or within a base or prime editor.

TABLE 4A
List of Exemplary Cas9 or Cas12a Orthologs

| | UniProt or GenBank | Nickase Mutations/Catalytic |
|---|---|---|
| Ortholog | Accession Number | residues |
| S. pyogenes Cas9 (SpCas9) | Q99ZW2.1 | D10A, E762A, H840A, N854A, |
| | | N863A, D986A¹⁷ |
| S. aureus Cas9 (SaCas9) | J7RUA5.1 | D10A and N580¹⁸ |
| S. thermophilus Cas9 | G3ECR1.2 | D31A and N891A¹⁹ |
| (St1Cas9) | | |
| S. pasteurianus Cas9 | BAK30384.1 | D10, H599* |
| (SpaCas9) | | |
| C. jejuni Cas9 (CjCas9) | Q0P897.1 | D8A, H559A²⁰ |
| F. novicida Cas9 (FnCas9) | A0Q5Y3.1 | D11, N995²¹ |
| P. lavamentivorans Cas9 | A7HP89.1 | D8, H601* |
| (PlCas9) | | |
| C. lari Cas9 (ClCas9) | G1UFN3.1 | D7, H567* |
| Pasteurella multocida Cas9 | Q9CLT2.1 | |
| F. novicida Cpf1 (FnCpf1) | A0Q7Q2.1 | D917, E1006, D1255²¹ |
| M. bovoculi Cpf1 (MbCpf1) | WP 052585281.1 | D986A** |
| A. sp. BV3L6 Cpf1 (AsCpf1) | U2UMQ6.1 | D908, 993E, Q1226, D1263²³ |
| L. bacterium N2006 (LbCpf1) | A0A182DWE3.1 | D832A²⁴ |

*predicted based on UniRule annotation on the UniProt database.
**Unpublished but deposited at addgene by Ervin Welker: pTE4565 (Addgene plasmid No 88903)

TABLE 4B
List of Exemplary High Fidelity and/or PAM-relaxed RGN Orthologs

| Published HF/PAM- | | |
|---|---|---|
| RGN variants | PMID | Mutations* |
| S. pyogenes Cas9 | 26628643 | K810A/K1003A/R1060A (1.0); |
| (SpCas9) eSpCas9 | | K848A/K1003A/R1060A(1.1) |
| S. pyogenes Cas9 | 29431739 | M495V/Y515N/K526E/R661Q; |
| (SpCas9) evoCas9 | | (M495V/Y515N/K526E/R661S; |
| | | M495V/Y515N/K526E/R661L) |
| S. pyogenes Cas9 | 26735016 | N497A/R661A/Q695A/Q926A |
| (SpCas9) HF1 | | |
| S. pyogenes Cas9 | 30082871 | R691A |
| (SpCas9) HiFi Cas9 | | |
| S. pyogenes Cas9 | 28931002 | N692A, M694A, Q695A, H698A |
| (SpCas9) HypaCas9 | | |
| S. pyogenes Cas9 | 30082838 | F539S, M763I, K890N |
| (SpCas9) Sniper- | | |
| Cas9 | | |
| S. pyogenes Cas9 | 29512652 | A262T, R324L, S409I, E480K, E543D, M694I, E1219V |
| (SpCas9) xCas9 | | |
| S. pyogenes Cas9 | 30166441 | R1335V, L1111R, D1135V, G1218R, |
| (SpCas9) SpCas9- | | E1219F, A1322R, T1337R |
| NG | | |
| S. pyogenes Cas9 | 26098369 | D1135V, R1335Q, T1337R; |
| (SpCas9) | | D1135V/G1218R/R1335E/T1337R |
| VQR/VRER | | |
| S. aureus Cas9 | 26524662 | E782K/N968K/R1015H |
| (SaCas9)-KKH | | |
| enAsCas12a | USSN | One or more of: E174R, S170R, S542R, K548R, |
| | 15/960,271 | K548V, N551R, N552R, K607R, K607H, e.g., |
| | | E174R/S542R/K548R, E174R/S542R/K607R, |
| | | E174R/S542R/K548V/N552R, S170R/S542R/K548R, |
| | | S170R/E174R, E174R/S542R, S170R/S542R, |
| | | E174R/S542R/K548R/N551R, E174R/S542R/K607H, |
| | | S170R/S542R/K607R, or |
| | | S170R/S542R/K548V/N552R |
| enAsCas12a-HF | USSN | One or more of: E174R, S542R, K548R, e.g., |
| | 15/960,271 | E174R/S542R/K548R, E174R/S542R/K607R, |
| | | E174R/S542R/K548V/N552R, S170R/S542R/K548R, |
| | | S170R/E174R, E174R/S542R, S170R/S542R, |
| | | E174R/S542R/K548R/N551R, E174R/S542R/K607H, |
| | | S170R/S542R/K607R, or |
| | | S170R/S542R/K548V/N552R, with the addition of one |
| | | or more of: N282A, T315A, N515A and K949A |
| enLbCas12a(HF) | USSN | One or more of T152R, T152K, D156R, D156K, |
| | 15/960,271 | Q529K, G532R, G532K, G532Q, K538R, K538V, |
| | | D541R, Y542R, M592A, K595R, K595H, K595S or |
| | | K595Q, e.g., D156R/G532R/K538R, |
| | | D156R/G532R/K595R, D156R/G532R/K538V/Y542R, |
| | | T152R/G532R/K538R, T152R/D156R, D156R/G532R, |
| | | T152R/G532R, D156R/G532R/K538R/D541R, |
| | | D156R/G532R/K595H, T152R/G532R/K595R, |
| | | T152R/G532R/K538V/Y542R, optionally with the |
| | | addition of one or more of: N260A, N256A, K514A, |
| | | D505A, K881A, S286A, K272A, K897A |
| enFnCas12a(HF) | USSN | One or more of T177A, K180R, K180K, E184R, |
| | 15/960,271 | E184K, T604K, N607R, N607K, N607Q, K613R, |
| | | K613V, D616R, N617R, M668A, K671R, K671H, |
| | | K671S, or K671Q, e.g., E184R/N607R/K613R, |
| | | E184R/N607R/K671R, E184R/N607R/K613V/N617R, |
| | | K180R/N607R/K613R, K180R/E184R, E184R/N607R, |
| | | K180R/N607R, E184R/N607R/K613R/D616R, |
| | | E184R/N607R/K671H, K180R/N607R/K671R, |
| | | K180R/N607R/K613V/N617R, optionally with the |
| | | addition of one or more of: N305A, N301A, K589A, |
| | | N580A, K962A, S334A, K320A, K978A |
| chimeric Cas9 | 30718489 | S. aureus Cas9 with PAM interaction domain from |
| cCas9 | | SaCas9 orthologues, expands recognition and |
| | | targetability of NNVRRN, NNVACT, NNVATG, |
| | | NNVATT, NNVGCT, NNVGTG, and NNVGTT PAM |
| | | sequences |
| Streptococcus | doi.org/10.1 | Recognizes 5’-NAA-3’ PAM |
| macacae (Smac) | 101/429654 | |
| Cas9 NCTC 11558 | | |
| Spy-mac Cas9, | doi.org/10.1 | Recognizes 5’-NAA-3’ PAM |
| Smac-py Cas9 | 101/429654 | |
| N. meningitidis | 30581144 | Recognizes N₄CC PAM |
| Nme2Cas9 | | |
| SpG Cas9 (SEQ-ID | 32217751 | SpCas9 variant capable of targeting NGN PAMs |
| 158) | | D1135L/S1136W/G1218K/E1219Q/R1335Q/T1337R |
| | | Also as SpG-HF1 in combination with |
| | | N497A/R661A/Q695A/Q926A |
| SpRY Cas9 (SEQ-ID | 32217751 | SpCas9 variant capable of targeting NRN>NYN PAMs |
| 157) | | SpRY(A61R/L1111R/D1135L/S1136W/G1218K/E121 |
| | | 9Q/N1317R/A1322R/R1333P/R1335Q/T1337R); also |
| | | as SpRY-HF1 in combination with |
| | | N497A/R661A/Q695A/Q926A |

*predicted based on UniRule annotation on the UniProt database.

SHAPE Applications

Examples of applications of the SHAPE strategy include SHAPE-TF (transcription factor), SHAPE-UTR (untranslated region), SHAPE-RNAi (RNA interference), and SHAPE-protein (e.g., Kozak sequence, codon optimization).

SHAPE-TF

In some instances, the SHAPE strategy can alter target gene expression at the pre-transcriptional level through the targeted introduction of transcription factor binding motifs (SHAPE-TF) into regions in the genome. Following the introduction of these transcription factor binding motifs (e.g., identified using a method described herein, or from a database such as the JASPAR database (e.g., 8th release—Fornes et al, “JASPAR 2020: update of the open-access database of transcription factor binding profiles”, Nucleic Acids Research Volume 48, Issue D1, 8 Jan. 2020, Pages D87—D92.), the recruitment of endogenous transcription factors to the target locus can enable the activation or repression of target gene expression through the increase or decrease in gene transcription, respectively. In addition to the introduction or modification of transcription factor binding sites, SHAPE-TF can also work through the modification of spacing between endogenous transcription factor binding sites to achieve alteration of target gene expression. To achieve SHAPE-TF, there are several steps that include 1) the identification/discovery of sequence motifs that can actively recruit transcription factors or modify endogenous transcription factor spacing, respectively, 2) the identification of target genomic sequence(s) to introduce the sequence motif or modification to affect target gene expression, and 3) identification of the genetic modifier to use to install the precise edit.

SHAPE-UTR

In some instances, the SHAPE strategy can alter target gene expression at the post-transcriptional level through the targeted introduction of sequence motifs into untranslated regions (SHAPE-UTR) of target gene transcripts to affect transcript stability and downstream protein expression. Following the introduction of these sequence motifs into UTRs, the stabilization or de-stabilization of target gene transcripts can enable the activation or repression of target gene expression through the increase or decrease in gene translation, respectively. To achieve SHAPE-UTR, there are several steps that include 1) the identification or discovery of sequence motifs that can modify a transcript stability when placed at the 5′ and/or 3′ UTRs, 2) the identification of specific regions within the 5′ and/or 3′ UTRs to introduce a sequence motif to affect transcript stability, and 3) identification of the genetic modifier to use to install the precise edit.

SHAPE-RNAi

In some instances, the SHAPE strategy can alter target gene expression at the post-transcriptional level through the targeted introduction of sequence motifs (e.g., microRNA binding sites on DNA/RNA, e.g., from the mirtarbase database (e.g., miRTarBase Release 8.0—Chou et al, “miRTarBase update 2018: A resource for experimentally validated microRNA-target interactions” Nucleic Acids Research 2018 Jan. 4; 46(D1):D296-D302)) that are targeted by endogenous non-coding RNAs (e.g., miRNAs, siRNAs, lncRNAs) to achieve RNA interference (SHAPE-RNAi). Following the introduction of these sequence motifs, endogenous non-coding RNAs may bind the introduced sequence and de-stabilize or inhibit translation of the target transcripts. To achieve SHAPE-RNAi, there are several steps that include 1) the identification or discovery of sequence motifs that can be targeted by endogenous non-coding RNA molecules, 2) the identification of specific regions in the target transcript that can promote RNA interference, and 3) identification of the genetic modifier to use to install the precise edit.

SHAPE-Protein

In some instances, the SHAPE strategy can alter target gene expression at the post-transcriptional level through sequence optimization for target gene transcripts (SHAPE-protein) to increase translation efficiency. The introduction of sequence motifs can be performed through the de novo introduction of new regulatory elements or optimization of endogenous regulatory elements that play a role in transcription or translation initiation and/or elongation. These sequence motifs can be related to elements such as the Kozak sequence or optimal codon structures for the coding regions of target gene transcripts. To achieve SHAPE-protein, there are several steps that include 1) the identification or discovery of sequence motifs that can modify translation initiation and/or elongation efficiency, 2) the identification of specific regions in genome to introduce a sequence motif for modification of translation initiation and/or elongation, and 3) identification of the genetic modifier to use to install the precise edit.

### Methods of Altering Expression of Disease-Related Target Genes

The present methods can include using SHAPE to alter expression of disease related target genes. Thus in some embodiments, the target gene is associated with a disease. Examples of haploinsufficiency diseases (Table 5; from Matharu N, Rattanasopha S, Tamura S, et al. Science. 2019; 363(6424)) and diseases caused by non-coding mutations (Table 6) are listed below. Rather than correcting a mutation that leads to haploinsufficiency, the present methods can be used to rescue haploinsufficiency with synthetic upregulation of a healthy allele—without reverting the “damaged” allele to WT sequence. Table 6 shows disease-related enhancer SNPs that cause other diseases based on down-regulated or upregulated expression. Again, rather than correcting the exact SNPs/genetic variants that cause disease, the present methods can be used to mitigate the effects of those SNPs.

TABLE 5
Examples of genes leading to haploinsufficiency
disease targetable by SHAPE

| Gene name | Gene ID PMID | Transcript ID |
|---|---|---|
| AAGAB | ENSG00000103591 | ENST00000261880 |
| ABCA4 | ENSG00000198691 | ENST00000370225 |
| ABCA7 | ENSG00000064687 | ENST00000263094 |
| ABCC6 | ENSG00000275331 | ENST00000620078 |
| ABCC9 | ENSG00000069431 | ENST00000261201 |
| ABCD1 | ENSG00000101986 | ENST00000218104 |
| ACSL4 | ENSG00000068366 | ENST00000340800 |
| ACTB | ENSG00000075624 | ENST00000331789 |
| ACTC1 | ENSG00000159251 | ENST00000290378 |
| ACTN2 | ENSG00000077522 | ENST00000366578 |
| ACVRL1 | ENSG00000139567 | ENST00000388922 |
| ADAR | ENSG00000160710 | ENST00000368474 |
| AFF2 | ENSG00000155966 | ENST00000370460 |
| AFG3L2 | ENSG00000141385 | ENST00000269143 |
| AGRP | ENSG00000159723 | ENST00000290953 |
| AHSP | ENSG00000169877 | ENST00000302312 |
| AKT3 | ENSG00000275199 | ENST00000613395 |
| ALDH1A2 | ENSG00000128918 | ENST00000249750 |
| ALX4 | ENSG00000052850 | ENST00000329255 |
| ANK2 | ENSG00000145362 | ENST00000503423 |
| ANKRD11 | ENSG00000167522 | ENST00000301030 |
| ANOS1 | ENSG00000011201 | ENST00000262648 |
| ANXA7 | ENSG00000138279 | ENST00000372921 |
| AP1S2 | ENSG00000182287 | ENST00000329235 |
| APC | ENSG00000134982 | ENST00000257430 |
| AR | ENSG00000169083 | ENST00000374690 |
| ARID2 | ENSG00000189079 | ENST00000334344 |
| ARSE | ENSG00000157399 | ENST00000381134 |
| ARX | ENSG00000004848 | ENST00000379044 |
| ASXL1 | ENSG00000171456 | ENST00000375687 |
| ASXL2 | ENSG00000143970 | ENST00000435504 |
| ATM | ENSG00000149311 | ENST00000278616 |
| ATP1A1 | ENSG00000163399 | ENST00000295598 |
| ATP1A2 | ENSG00000018625 | ENST00000361216 |
| ATP2A2 | ENSG00000174437 | ENST00000539276 |
| ATP2C1 | ENSG00000017260 | ENST00000359644 |
| ATP7A | ENSG00000165240 | ENST00000341514 |
| ATP8A2 | ENSG00000132932 | ENST00000381655 |
| ATR | ENSG00000175054 | ENST00000350721 |
| ATRX | ENSG00000085224 | ENST00000373344 |
| AUTS2 | ENSG00000158321 | ENST00000342771 |
| AVPR2 | ENSG00000126895 | ENST00000358927 |
| B4GALT7 | ENSG00000027847 | ENST00000029410 |
| BAG1 | ENSG00000107262 | ENST00000634734 |
| BAG3 | ENSG00000151929 | ENST00000369085 |
| BCL11A | ENSG00000119866 | ENST00000335712 |
| BCL11B | ENSG00000127152 | ENST00000357195 |
| BCO1 | ENSG00000135697 | ENST00000258168 |
| BCOR | ENSG00000183337 | ENST00000342274 |
| BECN1 | ENSG00000126581 | ENST00000361523 |
| BLM | ENSG00000197299 | ENST00000355112 |
| BMP4 | ENSG00000125378 | ENST00000559087 |
| BMPR1A | ENSG00000107779 | ENST00000372037 |
| BMPR2 | ENSG00000204217 | ENST00000374580 |
| BRAF | ENSG00000157764 | ENST00000288602 |
| BRCA1 | ENSG00000012048 | ENST00000471181 |
| BRCA2 | ENSG00000139618 | ENST00000380152 |
| BRWD3 | ENSG00000165288 | ENST00000373275 |
| BTK | ENSG00000010671 | ENST00000308731 |
| BUB1B | ENSG00000156970 | ENST00000287598 |
| BUB3 | ENSG00000154473 | ENST00000368865 |
| C3 | ENSG00000125730 | ENST00000245907 |
| CACNA1A | ENSG00000141837 | ENST00000360228 |
| CACNA1C | ENSG00000151067 | ENST00000347598 |
| CACNB2 | ENSG00000165995 | ENST00000324631 |
| CAMTA1 | ENSG00000171735 | ENST00000303635 |
| CASK | ENSG00000147044 | ENST00000378166 |
| CAV1 | ENSG00000105974 | ENST00000341049 |
| CAV3 | ENSG00000182533 | ENST00000343849 |
| CBFB | ENSG00000067955 | ENST00000290858 |
| CCNA2 | ENSG00000145386 | ENST00000618014 |
| CD2AP | ENSG00000198087 | ENST00000359314 |
| CDC73 | ENSG00000134371 | ENST00000367435 |
| CDH1 | ENSG00000039068 | ENST00000261769 |
| CDKL5 | ENSG00000008086 | ENST00000379989 |
| CDKN1B | ENSG00000111276 | ENST00000228872 |
| CDKN1C | ENSG00000273707 | ENST00000616138 |
| CDKN2A | ENSG00000147889 | ENST00000304494 |
| CDKN2B | ENSG00000147883 | ENST00000276925 |
| CDKN2C | ENSG00000123080 | ENST00000262662 |
| CEP152 | ENSG00000103995 | ENST00000399334 |
| CHD2 | ENSG00000173575 | ENST00000577670 |
| CHD5 | ENSG00000116254 | ENST00000262450 |
| CHD7 | ENSG00000171316 | ENST00000423902 |
| CHD8 | ENSG00000100888 | ENST00000430710 |
| CHL1 | ENSG00000134121 | ENST00000256509 |
| CHM | ENSG00000188419 | ENST00000357749 |
| CHRDL1 | ENSG00000101938 | ENST00000394797 |
| CHRM3 | ENSG00000133019 | ENST00000255380 |
| CHRNB2 | ENSG00000160716 | ENST00000368476 |
| CIC | ENSG00000079432 | ENST00000575354 |
| CLCN5 | ENSG00000171365 | ENST00000376108 |
| CNKSR2 | ENSG00000149970 | ENST00000425654 |
| CNTN4 | ENSG00000144619 | ENST00000418658 |
| CNTNAP2 | ENSG00000278728 | ENST00000613345 |
| COCH | ENSG00000100473 | ENST00000396618 |
| COL10A1 | ENSG00000123500 | ENST00000243222 |
| COL11A1 | ENSG00000060718 | ENST00000370096 |
| COL1A1 | ENSG00000108821 | ENST00000225964 |
| COL1A2 | ENSG00000164692 | ENST00000297268 |
| COL2A1 | ENSG00000139219 | ENST00000380518 |
| COL3A1 | ENSG00000168542 | ENST00000304636 |
| COL4A1 | ENSG00000187498 | ENST00000375820 |
| COL5A1 | ENSG00000130635 | ENST00000371817 |
| COL6A1 | ENSG00000142156 | ENST00000361866 |
| COMT | ENSG00000093010 | ENST00000585066 |
| COPS3 | ENSG00000141030 | ENST00000268717 |
| COPS5 | ENSG00000121022 | ENST00000357849 |
| CREBBP | ENSG00000005339 | ENST00000262367 |
| CRX | ENSG00000105392 | ENST00000221996 |
| CRYAB | ENSG00000109846 | ENST00000527950 |
| CRYBB2 | ENSG00000244752 | ENST00000398215 |
| CSF1R | ENSG00000182578 | ENST00000286301 |
| CSF2RA | ENSG00000198223 | ENST00000417535 |
| CSH1 | ENSG00000136488 | ENST00000316193 |
| CSRP3 | ENSG00000129170 | ENST00000533783 |
| CTCF | ENSG00000102974 | ENST00000264010 |
| CTLA4 | ENSG00000163599 | ENST00000302823 |
| CTNND2 | ENSG00000169862 | ENST00000304623 |
| CUL4B | ENSG00000158290 | ENST00000404115 |
| CYBB | ENSG00000165168 | ENST00000378588 |
| CYP11A1 | ENSG00000140459 | ENST00000268053 |
| DCC | ENSG00000187323 | ENST00000442544 |
| DCX | ENSG00000077279 | ENST00000356220 |
| DDX3X | ENSG00000215301 | ENST00000629496 |
| DEAF1 | ENSG00000282712 | ENST00000634194 |
| DEPDC5 | ENSG00000100150 | ENST00000382112 |
| DFFB | ENSG00000169598 | ENST00000378209 |
| DFNA5 | ENSG00000105928 | ENST00000342947 |
| DGCR2 | ENSG00000070413 | ENST00000263196 |
| DICER1 | ENSG00000100697 | ENST00000526495 |
| DIRC2 | ENSG00000138463 | ENST00000261038 |
| DKC1 | ENSG00000130826 | ENST00000626756 |
| DLG3 | ENSG00000082458 | ENST00000374360 |
| DLL4 | ENSG00000128917 | ENST00000249749 |
| DMD | ENSG00000198947 | ENST00000357033 |
| DMPK | ENSG00000104936 | ENST00000291270 |
| DMRT1 | ENSG00000137090 | ENST00000382276 |
| DMRT2 | ENSG00000173253 | ENST00000382251 |
| DSC2 | ENSG00000134755 | ENST00000280904 |
| DSG1 | ENSG00000134760 | ENST00000257192 |
| DSG2 | ENSG00000046604 | ENST00000261590 |
| DSP | ENSG00000096696 | ENST00000379802 |
| DYRK1A | ENSG00000157540 | ENST00000398960 |
| EBF3 | ENSG00000108001 | ENST00000368648 |
| EBP | ENSG00000147155 | ENST00000495186 |
| EDA | ENSG00000158813 | ENST00000374553 |
| EDN3 | ENSG00000124205 | ENST00000337938 |
| EDNRB | ENSG00000136160 | ENST00000334286 |
| EEF1E1 | ENSG00000124802 | ENST00000379715 |
| EFNB1 | ENSG00000090776 | ENST00000204961 |
| EFTUD2 | ENSG00000108883 | ENST00000426333 |
| EGR1 | ENSG00000120738 | ENST00000239938 |
| EHHADH | ENSG00000113790 | ENST00000231887 |
| EHMT1 | ENSG00000181090 | ENST00000629335 |
| ELAVL4 | ENSG00000162374 | ENST00000371819 |
| ELN | ENSG00000049540 | ENST00000252034 |
| ELOVL4 | ENSG00000118402 | ENST00000369816 |
| EMX2 | ENSG00000170370 | ENST00000553456 |
| ENAM | ENSG00000132464 | ENST00000396073 |
| ENG | ENSG00000106991 | ENST00000344849 |
| EP300 | ENSG00000100393 | ENST00000408233 |
| ERF | ENSG00000105722 | ENST00000222329 |
| ERMARD | ENSG00000276187 | ENST00000621205 |
| ETV6 | ENSG00000139083 | ENST00000396373 |
| EXT1 | ENSG00000182197 | ENST00000378204 |
| EXT2 | ENSG00000151348 | ENST00000533608 |
| EYA1 | ENSG00000104313 | ENST00000340726 |
| EYA4 | ENSG00000112319 | ENST00000531901 |
| F8 | ENSG00000185010 | ENST00000360256 |
| F9 | ENSG00000101981 | ENST00000218099 |
| FAM58A | ENSG00000262919 | ENST00000440428 |
| FANCB | ENSG00000181544 | ENST00000324138 |
| FAS | ENSG00000026103 | ENST00000355740 |
| FBLN1 | ENSG00000077942 | ENST00000327858 |
| FBN1 | ENSG00000166147 | ENST00000316623 |
| FBN2 | ENSG00000138829 | ENST00000508053 |
| FBXO38 | ENSG00000145868 | ENST00000340253 |
| FBXW7 | ENSG00000109670 | ENST00000281708 |
| FECH | ENSG00000066926 | ENST00000262093 |
| FEN1 | ENSG00000168496 | ENST00000305885 |
| FGD1 | ENSG00000102302 | ENST00000375135 |
| FGF10 | ENSG00000070193 | ENST00000264664 |
| FGF3 | ENSG00000186895 | ENST00000334134 |
| FGF8 | ENSG00000107831 | ENST00000320185 |
| FGFR1 | ENSG00000077782 | ENST00000447712 |
| FGFR3 | ENSG00000068078 | ENST00000440486 |
| FIGLA | ENSG00000183733 | ENST00000332372 |
| FKBP6 | ENSG00000077800 | ENST00000252037 |
| FLCN | ENSG00000154803 | ENST00000285071 |
| FLG | ENSG00000143631 | ENST00000368799 |
| FLI1 | ENSG00000151702 | ENST00000527786 |
| FLNA | ENSG00000196924 | ENST00000422373 |
| FOXC1 | ENSG00000054598 | ENST00000380874 |
| FOXC2 | ENSG00000176692 | ENST00000320354 |
| FOXE3 | ENSG00000186790 | ENST00000335071 |
| FOXF1 | ENSG00000103241 | ENST00000262426 |
| FOXG1 | ENSG00000176165 | ENST00000313071 |
| FOXL2 | ENSG00000183770 | ENST00000330315 |
| FOXO1 | ENSG00000150907 | ENST00000379561 |
| FOXP1 | ENSG00000114861 | ENST00000318789 |
| FOXP2 | ENSG00000128573 | ENST00000393491 |
| FRMD7 | ENSG00000165694 | ENST00000298542 |
| FSCN2 | ENSG00000186765 | ENST00000417245 |
| FSHR | ENSG00000170820 | ENST00000406846 |
| FTL | ENSG00000087086 | ENST00000331825 |
| FTSJ1 | ENSG00000068438 | ENST00000348411 |
| FUS | ENSG00000089280 | ENST00000254108 |
| FZD4 | ENSG00000174804 | ENST00000531380 |
| GALR1 | ENSG00000166573 | ENST00000299727 |
| GATA2 | ENSG00000179348 | ENST00000341105 |
| GATA3 | ENSG00000107485 | ENST00000346208 |
| GATA4 | ENSG00000136574 | ENST00000335135 |
| GATA6 | ENSG00000141448 | ENST00000269216 |
| GCH1 | ENSG00000131979 | ENST00000491895 |
| GCK | ENSG00000106633 | ENST00000403799 |
| GCKR | ENSG00000084734 | ENST00000264717 |
| GCNT1 | ENSG00000187210 | ENST00000442371 |
| GDF5 | ENSG00000125965 | ENST00000374372 |
| GDI1 | ENSG00000203879 | ENST00000447750 |
| GDNF | ENSG00000168621 | ENST00000515058 |
| GHRL | ENSG00000157017 | ENST00000335542 |
| GJA5 | ENSG00000265107 | ENST00000621517 |
| GJA8 | ENSG00000121634 | ENST00000369235 |
| GK | ENSG00000198814 | ENST00000378943 |
| GLA | ENSG00000102393 | ENST00000218516 |
| GLI2 | ENSG00000074047 | ENST00000452319 |
| GLI3 | ENSG00000106571 | ENST00000395925 |
| GLMN | ENSG00000174842 | ENST00000370360 |
| GNAS | ENSG00000087460 | ENST00000371102 |
| GPC3 | ENSG00000147257 | ENST00000370818 |
| GREB1L | ENSG00000141449 | ENST00000424526 |
| GRIA3 | ENSG00000125675 | ENST00000620443 |
| GRIN1 | ENSG00000176884 | ENST00000371553 |
| GRIN2A | ENSG00000183454 | ENST00000396573 |
| GRIN2B | ENSG00000273079 | ENST00000609686 |
| GRN | ENSG00000030582 | ENST00000053867 |
| GTF2I | ENSG00000263001 | ENST00000573035 |
| GTF2IRD1 | ENSG00000006704 | ENST00000265755 |
| H2AFX | ENSG00000188486 | ENST00000530167 |
| HCCS | ENSG00000004961 | ENST00000321143 |
| HCN4 | ENSG00000138622 | ENST00000261917 |
| HDAC4 | ENSG00000068024 | ENST00000345617 |
| HIC1 | ENSG00000177374 | ENST00000322941 |
| HIRA | ENSG00000100084 | ENST00000263208 |
| HIVEP2 | ENSG00000010818 | ENST00000367603 |
| HMGA1 | ENSG00000137309 | ENST00000311487 |
| HMGA2 | ENSG00000149948 | ENST00000403681 |
| HNF1A | ENSG00000135100 | ENST00000257555 |
| HNF1B | ENSG00000276194 | ENST00000617811 |
| HNF4A | ENSG00000101076 | ENST00000609795 |
| HNRNPK | ENSG00000165119 | ENST00000384871 |
| HNRNPU | ENSG00000153187 | ENST00000475997 |
| HOXD13 | ENSG00000128714 | ENST00000392539 |
| HPRT1 | ENSG00000165704 | ENST00000298556 |
| HRG | ENSG00000113905 | ENST00000232003 |
| HUWE1 | ENSG00000086758 | ENST00000262854 |
| ID2 | ENSG00000115738 | ENST00000234091 |
| IDS | ENSG00000010404 | ENST00000340855 |
| IGF1 | ENSG00000017427 | ENST00000337514 |
| IGF1R | ENSG00000140443 | ENST00000268035 |
| IGFALS | ENSG00000099769 | ENST00000215539 |
| IKBKG | ENSG00000269335 | ENST00000594239 |
| IKZF1 | ENSG00000185811 | ENST00000331340 |
| IL1RAPL1 | ENSG00000169306 | ENST00000378993 |
| IRF3 | ENSG00000126456 | ENST00000377139 |
| IRF6 | ENSG00000117595 | ENST00000367021 |
| JAG1 | ENSG00000101384 | ENST00000254958 |
| KANSL1 | ENSG00000278458 | ENST00000627698 |
| KAT6B | ENSG00000156650 | ENST00000287239 |
| KCNAB2 | ENSG00000069424 | ENST00000378097 |
| KCNE1 | ENSG00000180509 | ENST00000337385 |
| KCNE2 | ENSG00000159197 | ENST00000290310 |
| KCNH2 | ENSG00000055118 | ENST00000262186 |
| KCNJ18 | ENSG00000260458 | ENST00000567955 |
| KCNJ2 | ENSG00000123700 | ENST00000243457 |
| KCNQ1 | ENSG00000282076 | ENST00000632153 |
| KCNQ2 | ENSG00000281151 | ENST00000627948 |
| KCNQ4 | ENSG00000117013 | ENST00000347132 |
| KDM5C | ENSG00000126012 | ENST00000375401 |
| KDM6A | ENSG00000147050 | ENST00000377967 |
| KHDRBS2 | ENSG00000112232 | ENST00000281156 |
| KIF11 | ENSG00000138160 | ENST00000260731 |
| KIF1B | ENSG00000054523 | ENST00000263934 |
| KLF1 | ENSG00000105610 | ENST00000264834 |
| KLF6 | ENSG00000067082 | ENST00000497571 |
| KLHL10 | ENSG00000161594 | ENST00000293303 |
| KMT2A | ENSG00000118058 | ENST00000389506 |
| KMT2B | ENSG00000272333 | ENST00000420124 |
| KMT2D | ENSG00000167548 | ENST00000301067 |
| KRAS | ENSG00000133703 | ENST00000256078 |
| KRIT1 | ENSG00000001631 | ENST00000394507 |
| KRT5 | ENSG00000186081 | ENST00000252242 |
| KRT74 | ENSG00000170484 | ENST00000305620 |
| L1CAM | ENSG00000198910 | ENST00000370060 |
| LAMA4 | ENSG00000112769 | ENST00000522006 |
| LAMP2 | ENSG00000005893 | ENST00000434600 |
| LDLR | ENSG00000130164 | ENST00000619864 |
| LEMD3 | ENSG00000174106 | ENST00000308330 |
| LETM1 | ENSG00000168924 | ENST00000302787 |
| LGI1 | ENSG00000108231 | ENST00000371418 |
| LHX4 | ENSG00000121454 | ENST00000263726 |
| LIG4 | ENSG00000174405 | ENST00000405925 |
| LIMK1 | ENSG00000106683 | ENST00000336180 |
| LMNA | ENSG00000160789 | ENST00000368300 |
| LMX1B | ENSG00000136944 | ENST00000373474 |
| LRP5 | ENSG00000162337 | ENST00000294304 |
| LZTR1 | ENSG00000099949 | ENST00000215739 |
| MAD2L1 | ENSG00000164109 | ENST00000296509 |
| MAGEL2 | ENSG00000254585 | ENST00000532292 |
| MAGT1 | ENSG00000102158 | ENST00000358075 |
| MAOA | ENSG00000189221 | ENST00000338702 |
| MAP2K1 | ENSG00000169032 | ENST00000307102 |
| MAP2K2 | ENSG00000126934 | ENST00000262948 |
| MAPK10 | ENSG00000109339 | ENST00000395169 |
| MAPKAPK3 | ENSG00000114738 | ENST00000446044 |
| MAPT | ENSG00000277956 | ENST00000626571 |
| MBD5 | ENSG00000204406 | ENST00000407073 |
| MC3R | ENSG00000124089 | ENST00000243911 |
| MC4R | ENSG00000166603 | ENST00000299766 |
| MECP2 | ENSG00000169057 | ENST00000303391 |
| MED13L | ENSG00000123066 | ENST00000281928 |
| MED15 | ENSG00000099917 | ENST00000292733 |
| MEF2C | ENSG00000081189 | ENST00000340208 |
| MEIS2 | ENSG00000134138 | ENST00000424352 |
| MEN1 | ENSG00000133895 | ENST00000394376 |
| MFAP5 | ENSG00000197614 | ENST00000359478 |
| MIB1 | ENSG00000101752 | ENST00000261537 |
| MID1 | ENSG00000101871 | ENST00000380787 |
| MITF | ENSG00000187098 | ENST00000394351 |
| MLH1 | ENSG00000076242 | ENST00000231790 |
| MLLT3 | ENSG00000171843 | ENST00000380338 |
| MME | ENSG00000196549 | ENST00000492661 |
| MNX1 | ENSG00000130675 | ENST00000469500 |
| MPZ | ENSG00000158887 | ENST00000533357 |
| MSH2 | ENSG00000095002 | ENST00000233146 |
| MSH6 | ENSG00000116062 | ENST00000234420 |
| MSX1 | ENSG00000163132 | ENST00000382723 |
| MSX2 | ENSG00000120149 | ENST00000239243 |
| MTAP | ENSG00000099810 | ENST00000380172 |
| MTM1 | ENSG00000171100 | ENST00000370396 |
| MUTYH | ENSG00000132781 | ENST00000372098 |
| MYBPC3 | ENSG00000134571 | ENST00000545968 |
| MYCN | ENSG00000134323 | ENST00000281043 |
| MYF6 | ENSG00000111046 | ENST00000228641 |
| MYH11 | ENSG00000133392 | ENST00000300036 |
| MYH7 | ENSG00000092054 | ENST00000355349 |
| MYH9 | ENSG00000100345 | ENST00000216181 |
| MYLK | ENSG00000065534 | ENST00000360304 |
| MYOC | ENSG00000034971 | ENST00000037502 |
| MYPN | ENSG00000138347 | ENST00000540630 |
| MYT1L | ENSG00000186487 | ENST00000428368 |
| NBN | ENSG00000104320 | ENST00000265433 |
| NCF1 | ENSG00000158517 | ENST00000289473 |
| NCSTN | ENSG00000162736 | ENST00000294785 |
| NDP | ENSG00000124479 | ENST00000378062 |
| NEBL | ENSG00000078114 | ENST00000417816 |
| NEXMIF | ENSG00000050030 | ENST00000055682 |
| NEXN | ENSG00000162614 | ENST00000334785 |
| NF1 | ENSG00000196712 | ENST00000356175 |
| NF2 | ENSG00000186575 | ENST00000338641 |
| NFIA | ENSG00000162599 | ENST00000403491 |
| NFIX | ENSG00000008441 | ENST00000397661 |
| NFKB1 | ENSG00000109320 | ENST00000226574 |
| NFKB2 | ENSG00000077150 | ENST00000369966 |
| NFKBIA | ENSG00000100906 | ENST00000216797 |
| NFRKB | ENSG00000170322 | ENST00000446488 |
| NHS | ENSG00000188158 | ENST00000380060 |
| NIPBL | ENSG00000164190 | ENST00000282516 |
| NKX2-1 | ENSG00000136352 | ENST00000518149 |
| NKX2-5 | ENSG00000183072 | ENST00000329198 |
| NKX3-1 | ENSG00000167034 | ENST00000380871 |
| NLGN4X | ENSG00000146938 | ENST00000381095 |
| NLRP3 | ENSG00000162711 | ENST00000391828 |
| NODAL | ENSG00000156574 | ENST00000287139 |
| NOG | ENSG00000183691 | ENST00000332822 |
| NOTCH1 | ENSG00000148400 | ENST00000277541 |
| NPAS3 | ENSG00000151322 | ENST00000346562 |
| NPM1 | ENSG00000181163 | ENST00000517671 |
| NPRL3 | ENSG00000103148 | ENST00000620134 |
| NR0B1 | ENSG00000169297 | ENST00000378970 |
| NR2F2 | ENSG00000185551 | ENST00000410719 |
| NR5A1 | ENSG00000136931 | ENST00000373588 |
| NRXN1 | ENSG00000179915 | ENST00000401669 |
| NSD1 | ENSG00000165671 | ENST00000439151 |
| NSDHL | ENSG00000147383 | ENST00000370274 |
| NT5E | ENSG00000135318 | ENST00000257770 |
| NUP98 | ENSG00000110713 | ENST00000324932 |
| NUS1 | ENSG00000153989 | ENST00000368494 |
| NXF5 | ENSG00000126952 | ENST00000473265 |
| NYX | ENSG00000188937 | ENST00000342595 |
| OCRL | ENSG00000122126 | ENST00000371113 |
| OFD1 | ENSG00000046651 | ENST00000340096 |
| OPA1 | ENSG00000198836 | ENST00000361908 |
| OPHN1 | ENSG00000079482 | ENST00000355520 |
| OTC | ENSG00000036473 | ENST00000039007 |
| OTX2 | ENSG00000165588 | ENST00000555006 |
| P2RX5 | ENSG00000083454 | ENST00000225328 |
| P2RY8 | ENSG00000182162 | ENST00000381297 |
| PAFAH1B1 | ENSG00000007168 | ENST00000397195 |
| PAK2 | ENSG00000180370 | ENST00000327134 |
| PAK3 | ENSG00000077264 | ENST00000446737 |
| PARK2 | ENSG00000185345 | ENST00000366898 |
| PAX1 | ENSG00000125813 | ENST00000398485 |
| PAX2 | ENSG00000075891 | ENST00000355243 |
| PAX3 | ENSG00000135903 | ENST00000392070 |
| PAX5 | ENSG00000196092 | ENST00000358127 |
| PAX6 | ENSG00000007372 | ENST00000379132 |
| PAX8 | ENSG00000125618 | ENST00000429538 |
| PAX9 | ENSG00000198807 | ENST00000402703 |
| PCDH19 | ENSG00000165194 | ENST00000255531 |
| PCGF2 | ENSG00000278644 | ENST00000631566 |
| PDHA1 | ENSG00000131828 | ENST00000545074 |
| PDLIM3 | ENSG00000154553 | ENST00000284770 |
| PGAP1 | ENSG00000197121 | ENST00000354764 |
| PGK1 | ENSG00000102144 | ENST00000373316 |
| PHB2 | ENSG00000215021 | ENST00000535923 |
| PHEX | ENSG00000102174 | ENST00000379374 |
| PHF6 | ENSG00000156531 | ENST00000370803 |
| PHF8 | ENSG00000172943 | ENST00000338154 |
| PHIP | ENSG00000146247 | ENST00000275034 |
| PHOX2B | ENSG00000109132 | ENST00000226382 |
| PIGA | ENSG00000165195 | ENST00000333590 |
| PIK3R1 | ENSG00000145675 | ENST00000521381 |
| PINK1 | ENSG00000158828 | ENST00000622012 |
| PITX2 | ENSG00000164093 | ENST00000306732 |
| PITX3 | ENSG00000107859 | ENST00000370002 |
| PKD1 | ENSG00000008710 | ENST00000612014 |
| PKD2 | ENSG00000118762 | ENST00000237596 |
| PKP2 | ENSG00000057294 | ENST00000340811 |
| PLIN1 | ENSG00000166819 | ENST00000430628 |
| PLK4 | ENSG00000142731 | ENST00000270861 |
| PLP1 | ENSG00000123560 | ENST00000612423 |
| PMP22 | ENSG00000109099 | ENST00000312280 |
| PNPLA6 | ENSG00000032444 | ENST00000221249 |
| POLG2 | ENSG00000256525 | ENST00000539111 |
| POLR1D | ENSG00000186184 | ENST00000302979 |
| POLR2F | ENSG00000100142 | ENST00000442738 |
| PORCN | ENSG00000102312 | ENST00000359882 |
| POT1 | ENSG00000128513 | ENST00000357628 |
| PPARA | ENSG00000186951 | ENST00000262735 |
| PPARG | ENSG00000132170 | ENST00000287820 |
| PQBP1 | ENSG00000102103 | ENST00000376563 |
| PRKAG2 | ENSG00000106617 | ENST00000287878 |
| PRKAR1A | ENSG00000108946 | ENST00000392711 |
| PRM1 | ENSG00000175646 | ENST00000312511 |
| PRM2 | ENSG00000122304 | ENST00000241808 |
| PRMT7 | ENSG00000132600 | ENST00000339507 |
| PRODH | ENSG00000100033 | ENST00000610940 |
| PROX1 | ENSG00000117707 | ENST00000498508 |
| PRPF31 | ENSG00000277953 | ENST00000622636 |
| PRPS1 | ENSG00000147224 | ENST00000372435 |
| PRRT2 | ENSG00000167371 | ENST00000358758 |
| PSMD12 | ENSG00000197170 | ENST00000356126 |
| PTCH1 | ENSG00000185920 | ENST00000331920 |
| PTEN | ENSG00000171862 | ENST00000371953 |
| PTHLH | ENSG00000087494 | ENST00000201015 |
| PTPN11 | ENSG00000179295 | ENST00000351677 |
| PUM1 | ENSG00000134644 | ENST00000257075 |
| QRICH1 | ENSG00000198218 | ENST00000395443 |
| RAB39B | ENSG00000155961 | ENST00000369454 |
| RAD50 | ENSG00000113522 | ENST00000378823 |
| RAD51 | ENSG00000051180 | ENST00000267868 |
| RAD51B | ENSG00000182185 | ENST00000471583 |
| RAE1 | ENSG00000101146 | ENST00000395841 |
| RAI1 | ENSG00000108557 | ENST00000353383 |
| RANGRF | ENSG00000108961 | ENST00000226105 |
| RASA1 | ENSG00000145715 | ENST00000274376 |
| RASSF1 | ENSG00000068028 | ENST00000357043 |
| RB1 | ENSG00000139687 | ENST00000267163 |
| RBFOX1 | ENSG00000078328 | ENST00000311745 |
| RBM20 | ENSG00000203867 | ENST00000369519 |
| RBPJ | ENSG00000168214 | ENST00000342295 |
| RELN | ENSG00000189056 | ENST00000343529 |
| RET | ENSG00000165731 | ENST00000355710 |
| RFC2 | ENSG00000049541 | ENST00000055077 |
| RNF135 | ENSG00000181481 | ENST00000328381 |
| ROBO1 | ENSG00000169855 | ENST00000495273 |
| ROR2 | ENSG00000169071 | ENST00000375708 |
| RP2 | ENSG00000102218 | ENST00000218340 |
| RPRM | ENSG00000177519 | ENST00000325926 |
| RPS17 | ENSG00000278229 | ENST00000617731 |
| RPS19 | ENSG00000105372 | ENST00000598742 |
| RPS20 | ENSG00000008988 | ENST00000009589 |
| RPS24 | ENSG00000138326 | ENST00000372360 |
| RPS26 | ENSG00000197728 | ENST00000356464 |
| RPS28 | ENSG00000233927 | ENST00000600659 |
| RPS29 | ENSG00000213741 | ENST00000245458 |
| RPS4X | ENSG00000198034 | ENST00000316084 |
| RPS6KA3 | ENSG00000177189 | ENST00000379565 |
| RS1 | ENSG00000102104 | ENST00000379984 |
| RTN2 | ENSG00000125744 | ENST00000344680 |
| RTN4R | ENSG00000040608 | ENST00000043402 |
| RUNX1 | ENSG00000159216 | ENST00000300305 |
| RUNX2 | ENSG00000124813 | ENST00000371432 |
| RYR1 | ENSG00000196218 | ENST00000359596 |
| RYR2 | ENSG00000198626 | ENST00000366574 |
| SALL1 | ENSG00000103449 | ENST00000251020 |
| SALL3 | ENSG00000277015 | ENST00000614883 |
| SALL4 | ENSG00000101115 | ENST00000217086 |
| SATB2 | ENSG00000119042 | ENST00000457245 |
| SBDS | ENSG00000126524 | ENST00000246868 |
| SCN10A | ENSG00000185313 | ENST00000449082 |
| SCN1A | ENSG00000144285 | ENST00000375405 |
| SCN2A | ENSG00000136531 | ENST00000631182 |
| SCN5A | ENSG00000183873 | ENST00000333535 |
| SCN8A | ENSG00000196876 | ENST00000354534 |
| SDHAF2 | ENSG00000167985 | ENST00000301761 |
| SDHB | ENSG00000117118 | ENST00000375499 |
| SDHC | ENSG00000143252 | ENST00000367975 |
| SDHD | ENSG00000204370 | ENST00000375549 |
| SEM1 | ENSG00000127922 | ENST00000248566 |
| SEMA3A | ENSG00000075213 | ENST00000265362 |
| SEMA5A | ENSG00000112902 | ENST00000382496 |
| SETBP1 | ENSG00000152217 | ENST00000282030 |
| SETD5 | ENSG00000168137 | ENST00000402198 |
| SF1 | ENSG00000168066 | ENST00000227503 |
| SF3B4 | ENSG00000143368 | ENST00000271628 |
| SGCE | ENSG00000127990 | ENST00000265735 |
| SH2B1 | ENSG00000178188 | ENST00000337120 |
| SH2D1A | ENSG00000183918 | ENST00000371139 |
| SH3BP2 | ENSG00000087266 | ENST00000452765 |
| SH3TC2 | ENSG00000169247 | ENST00000515425 |
| SHANK3 | ENSG00000251322 | ENST00000262795 |
| SHH | ENSG00000164690 | ENST00000297261 |
| SHMT1 | ENSG00000176974 | ENST00000617912 |
| SHOC2 | ENSG00000108061 | ENST00000369452 |
| SHOX | ENSG00000185960 | ENST00000381578 |
| SI | ENSG00000090402 | ENST00000264382 |
| SIM1 | ENSG00000112246 | ENST00000369208 |
| SIN3A | ENSG00000169375 | ENST00000360439 |
| SIX3 | ENSG00000138083 | ENST00000260653 |
| SIX6 | ENSG00000184302 | ENST00000327720 |
| SLC16A12 | ENSG00000152779 | ENST00000371790 |
| SLC16A2 | ENSG00000147100 | ENST00000587091 |
| SLC2A1 | ENSG00000117394 | ENST00000426263 |
| SLC33A1 | ENSG00000169359 | ENST00000392845 |
| SLC36A2 | ENSG00000186335 | ENST00000335244 |
| SLC40A1 | ENSG00000138449 | ENST00000261024 |
| SLC4A10 | ENSG00000144290 | ENST00000415876 |
| SLC52A1 | ENSG00000132517 | ENST00000424747 |
| SLC5A3 | ENSG00000198743 | ENST00000381151 |
| SLC5A7 | ENSG00000115665 | ENST00000264047 |
| SLC6A8 | ENSG00000130821 | ENST00000253122 |
| SLC9A3R1 | ENSG00000109062 | ENST00000581999 |
| SLC9A6 | ENSG00000198689 | ENST00000370695 |
| SMAD3 | ENSG00000166949 | ENST00000327367 |
| SMAD4 | ENSG00000141646 | ENST00000398417 |
| SMAD5 | ENSG00000113658 | ENST00000545620 |
| SMARCA4 | ENSG00000127616 | ENST00000589677 |
| SMARCB1 | ENSG00000275837 | ENST00000629690 |
| SMARCC1 | ENSG00000173473 | ENST00000254480 |
| SMS | ENSG00000102172 | ENST00000404933 |
| SNCA | ENSG00000145335 | ENST00000336904 |
| SNURF | ENSG00000273173 | ENST00000338327 |
| SOCS1 | ENSG00000185338 | ENST00000332029 |
| SON | ENSG00000159140 | ENST00000436227 |
| SOX10 | ENSG00000100146 | ENST00000360880 |
| SOX11 | ENSG00000176887 | ENST00000322002 |
| SOX18 | ENSG00000203883 | ENST00000340356 |
| SOX2 | ENSG00000181449 | ENST00000325404 |
| SOX5 | ENSG00000134532 | ENST00000546136 |
| SOX8 | ENSG00000005513 | ENST00000293894 |
| SOX9 | ENSG00000125398 | ENST00000245479 |
| SPAST | ENSG00000021574 | ENST00000615843 |
| SPINK1 | ENSG00000164266 | ENST00000296695 |
| SPR | ENSG00000116096 | ENST00000234454 |
| SPRED1 | ENSG00000166068 | ENST00000299084 |
| SPTBN2 | ENSG00000173898 | ENST00000533211 |
| SRGAP3 | ENSG00000196220 | ENST00000383836 |
| SRY | ENSG00000184895 | ENST00000383070 |
| ST7 | ENSG00000004866 | ENST00000622083 |
| STAT5A | ENSG00000126561 | ENST00000345506 |
| STAT5B | ENSG00000173757 | ENST00000293328 |
| STK11 | ENSG00000118046 | ENST00000326873 |
| STK25 | ENSG00000115694 | ENST00000316586 |
| STS | ENSG00000101846 | ENST00000217961 |
| STXBP1 | ENSG00000136854 | ENST00000373299 |
| SUMO1 | ENSG00000116030 | ENST00000392246 |
| SUZ12 | ENSG00000178691 | ENST00000322652 |
| SYN1 | ENSG00000008056 | ENST00000295987 |
| SYNGAP1 | ENSG00000227460 | ENST00000629380 |
| TAB2 | ENSG00000055208 | ENST00000367456 |
| TBK1 | ENSG00000183735 | ENST00000331710 |
| TBX1 | ENSG00000184058 | ENST00000332710 |
| TBX20 | ENSG00000164532 | ENST00000408931 |
| TBX22 | ENSG00000277800 | ENST00000619509 |
| TBX3 | ENSG00000135111 | ENST00000257566 |
| TBX4 | ENSG00000121075 | ENST00000240335 |
| TBX5 | ENSG00000089225 | ENST00000310346 |
| TCF12 | ENSG00000140262 | ENST00000438423 |
| TCF4 | ENSG00000196628 | ENST00000356073 |
| TCOF1 | ENSG00000070814 | ENST00000377797 |
| TDGF1 | ENSG00000241186 | ENST00000296145 |
| TERC | ENSG00000270141 | ENST00000602385 |
| TERT | ENSG00000164362 | ENST00000310581 |
| TFAP2B | ENSG00000008196 | ENST00000393655 |
| TFRC | ENSG00000072274 | ENST00000360110 |
| TGFB1 | ENSG00000105329 | ENST00000221930 |
| TGFBR1 | ENSG00000106799 | ENST00000374994 |
| TGFBR2 | ENSG00000163513 | ENST00000295754 |
| TGIF1 | ENSG00000177426 | ENST00000550958 |
| TIMM8A | ENSG00000126953 | ENST00000372902 |
| TKT | ENSG00000163931 | ENST00000462138 |
| TLK2 | ENSG00000146872 | ENST00000346027 |
| TLR3 | ENSG00000164342 | ENST00000296795 |
| TM4SF20 | ENSG00000168955 | ENST00000304568 |
| TMPO | ENSG00000120802 | ENST00000266732 |
| TNNI3 | ENSG00000129991 | ENST00000344887 |
| TNNT2 | ENSG00000118194 | ENST00000236918 |
| TNXA | ENSG00000224140 | ENST00000423622 |
| TNXB | ENSG00000236236 | ENST00000550539 |
| TOP3A | ENSG00000177302 | ENST00000542570 |
| TP53 | ENSG00000141510 | ENST00000269305 |
| TP53BP2 | ENSG00000143514 | ENST00000343537 |
| TP63 | ENSG00000073282 | ENST00000354600 |
| TP73 | ENSG00000078900 | ENST00000378295 |
| TPM1 | ENSG00000140416 | ENST00000288398 |
| TRAPPC2 | ENSG00000196459 | ENST00000359680 |
| TRIP12 | ENSG00000153827 | ENST00000283943 |
| TRPM4 | ENSG00000130529 | ENST00000252826 |
| TRPS1 | ENSG00000104447 | ENST00000395715 |
| TRPV4 | ENSG00000111199 | ENST00000261740 |
| TSC1 | ENSG00000165699 | ENST00000298552 |
| TSC2 | ENSG00000103197 | ENST00000219476 |
| TSPAN7 | ENSG00000156298 | ENST00000378482 |
| TTN | ENSG00000155657 | ENST00000589042 |
| TWIST1 | ENSG00000122691 | ENST00000242261 |
| TYRP1 | ENSG00000107165 | ENST00000388918 |
| UBE2A | ENSG00000077721 | ENST00000371558 |
| UBE3A | ENSG00000114062 | ENST00000232165 |
| UPF3B | ENSG00000125351 | ENST00000276201 |
| USP7 | ENSG00000187555 | ENST00000344836 |
| USP9X | ENSG00000124486 | ENST00000324545 |
| VAMP1 | ENSG00000139190 | ENST00000396308 |
| VEGFA | ENSG00000112715 | ENST00000425836 |
| VEGFC | ENSG00000150630 | ENST00000618562 |
| VHL | ENSG00000134086 | ENST00000256474 |
| WDR26 | ENSG00000162923 | ENST00000414423 |
| WDR45 | ENSG00000196998 | ENST00000356463 |
| WNT2B | ENSG00000134245 | ENST00000369684 |
| WT1 | ENSG00000184937 | ENST00000332351 |
| WWOX | ENSG00000186153 | ENST00000566780 |
| XIAP | ENSG00000101966 | ENST00000434753 |
| XRCC5 | ENSG00000079246 | ENST00000392133 |
| YAP1 | ENSG00000137693 | ENST00000282441 |
| YWHAE | ENSG00000274474 | ENST00000627231 |
| ZC4H2 | ENSG00000126970 | ENST00000374839 |
| ZDHHC9 | ENSG00000188706 | ENST00000357166 |
| ZEB2 | ENSG00000169554 | ENST00000627532 |
| ZFPM2 | ENSG00000169946 | ENST00000407775 |
| ZIC1 | ENSG00000152977 | ENST00000282928 |
| ZIC2 | ENSG00000043355 | ENST00000376335 |
| ZIC3 | ENSG00000156925 | ENST00000287538 |
| ZIC4 | ENSG00000174963 | ENST00000383075 |
| ZMPSTE24 | ENSG00000084073 | ENST00000372759 |
| ZNF41 | ENSG00000147124 | ENST00000377065 |
| ZNF462 | ENSG00000148143 | ENST00000277225 |
| ZNF674 | ENSG00000251192 | ENST00000523374 |
| ZNF711 | ENSG00000147180 | ENST00000373165 |

TABLE 6
Examples of functional non-coding mutations targetable by SHAPE

| Gene name | Chr | Start | Stop | Mutation | Consequence |
|---|---|---|---|---|---|
| A20 | chr6 | 138230039 | 138230039 | TT > A | Loss of NF-kB binding; |
| | | | | | Decreasing of enhancer |
| | | | | | activity |
| A20 | chr6 | 138230040 | 138230040 | TT > A | Loss of NF-kB binding; |
| | | | | | Decreasing of enhancer |
| | | | | | activity |
| ABCG2 | chr4 | 88924371 | 88924371 | A > G | Decreasing of enhancer |
| | | | | | activity |
| ABCG2 | chr4 | 89073197 | 89073197 | A > C | Decreasing of enhancer |
| | | | | | activity |
| ACSBG1 | chr15 | 78292380 | 78292380 | A > G | Decreasing of enhancer |
| | | | | | activity |
| ACSBG1 | chr15 | 78292544 | 78292544 | G > A | Decreasing of enhancer |
| | | | | | activity |
| ACSBG1 | chr15 | 78292823 | 78292823 | G > C | Decreasing of enhancer |
| | | | | | activity |
| ADAP1 | chr7 | 982031 | 982031 | G > C | Change of ATF5 binding |
| ADAP1 | chr7 | 982031 | 982031 | G > C | Change of BCL11A binding |
| ADAP1 | chr7 | 982031 | 982031 | G > C | Change of SPI1 binding |
| ADAP1 | chr7 | 982031 | 982031 | G > C | Change of ZNF227 binding |
| ADAP1 | chr7 | 982031 | 982031 | G > C | Change of ZNF263 binding |
| ADAP1 | chr7 | 982031 | 982031 | G > C | Change of ZNF281 binding |
| ADAP1 | chr7 | 982159 | 982159 | G > T | Change of ELF2 binding |
| ADAP1 | chr7 | 982159 | 982159 | G > T | Change of HOXC5 binding |
| ADAP1 | chr7 | 982159 | 982159 | G > T | Change of MAZ binding |
| ADAP1 | chr7 | 982159 | 982159 | G > T | Change of SPI1 binding |
| ADAP1 | chr7 | 982159 | 982159 | G > T | Change of TAF1 binding |
| ADAP1 | chr7 | 982159 | 982159 | G > T | Change of ZNF263 binding |
| ADAP1 | chr7 | 982159 | 982159 | G > T | Change of ZNF281 binding |
| ADAP1 | chr7 | 982159 | 982159 | G > T | Change of ZNF658B binding |
| ADAP1 | chr7 | 982233 | 982233 | G > A | Change of ATF5 binding |
| ADAP1 | chr7 | 982233 | 982233 | G > A | Change of BCL11A binding |
| ADAP1 | chr7 | 982233 | 982233 | G > A | Change of HOXC5 binding |
| ADAP1 | chr7 | 982233 | 982233 | G > A | Change of SPI1 binding |
| ADAP1 | chr7 | 982233 | 982233 | G > A | Change of ZNF227 binding |
| ADAP1 | chr7 | 982233 | 982233 | G > A | Change of ZNF263 binding |
| ADAP1 | chr7 | 982233 | 982233 | G > A | Change of ZNF658B binding |
| ADAP1 | chr7 | 982258 | 982258 | G > C | Change of ELF2 binding |
| ADAP1 | chr7 | 982258 | 982258 | G > C | Change of HOXC5 binding |
| ADAP1 | chr7 | 982258 | 982258 | G > C | Change of MAZ binding |
| ADAP1 | chr7 | 982258 | 982258 | G > C | Change of SPI1 binding |
| ADAP1 | chr7 | 982258 | 982258 | G > C | Change of TAF1 binding |
| ADAP1 | chr7 | 982258 | 982258 | G > C | Change of ZNF263 binding |
| ADAP1 | chr7 | 982258 | 982258 | G > C | Change of ZNF281 binding |
| ADAP1 | chr7 | 982258 | 982258 | G > C | Change of ZNF658B binding |
| ADAP1 | chr7 | 982269 | 982269 | G > A | Change of ELF2 binding |
| ADAP1 | chr7 | 982269 | 982269 | G > A | Change of HOXC5 binding |
| ADAP1 | chr7 | 982269 | 982269 | G > A | Change of MAZ binding |
| ADAP1 | chr7 | 982269 | 982269 | G > A | Change of SPI1 binding |
| ADAP1 | chr7 | 982269 | 982269 | G > A | Change of TAF1 binding |
| ADAP1 | chr7 | 982269 | 982269 | G > A | Change of ZNF263 binding |
| ADAP1 | chr7 | 982269 | 982269 | G > A | Change of ZNF658B binding |
| ADAP1 | chr7 | 982401 | 982401 | A > G | Change of HOXC5 binding |
| ADAP1 | chr7 | 982401 | 982401 | A > G | Change of MAZ binding |
| ADAP1 | chr7 | 982401 | 982401 | A > G | Change of SPI1 binding |
| ADAP1 | chr7 | 982401 | 982401 | A > G | Change of TAF1 binding |
| ADAP1 | chr7 | 982401 | 982401 | A > G | Change of ZNF263 binding |
| ADAP1 | chr7 | 982401 | 982401 | A > G | Change of ZNF658B binding |
| ADAP1 | chr7 | 982407 | 982407 | A > G | Change of HOXC5 binding |
| ADAP1 | chr7 | 982407 | 982407 | A > G | Change of MAZ binding |
| ADAP1 | chr7 | 982407 | 982407 | A > G | Change of SPI1 binding |
| ADAP1 | chr7 | 982407 | 982407 | A > G | Change of TAF1 binding |
| ADAP1 | chr7 | 982407 | 982407 | A > G | Change of ZNF263 binding |
| ADAP1 | chr7 | 982407 | 982407 | A > G | Change of ZNF658B binding |
| ADAP1 | chr7 | 982877 | 982877 | A > T | Change of ATF5 binding |
| ADAP1 | chr7 | 982877 | 982877 | A > T | Change of HOXC5 binding |
| ADAP1 | chr7 | 982877 | 982877 | A > T | Change of MAZ binding |
| ADAP1 | chr7 | 982877 | 982877 | A > T | Change of TAF1 binding |
| ADAP1 | chr7 | 982877 | 982877 | A > T | Change of ZNF227 binding |
| ADAP1 | chr7 | 982877 | 982877 | A > T | Change of ZNF263 binding |
| ADAP1 | chr7 | 982877 | 982877 | A > T | Change of ZNF281 binding |
| ADAP1 | chr7 | 982877 | 982877 | A > T | Change of ZNF658B binding |
| ADAP1 | chr7 | 982891 | 982891 | G > A | Change of ATF5 binding |
| ADAP1 | chr7 | 982891 | 982891 | G > A | Change of BCL11A binding |
| ADAP1 | chr7 | 982891 | 982891 | G > A | Change of HOXC5 binding |
| ADAP1 | chr7 | 982891 | 982891 | G > A | Change of MAZ binding |
| ADAP1 | chr7 | 982891 | 982891 | G > A | Change of SPI1 binding |
| ADAP1 | chr7 | 982891 | 982891 | G > A | Change of TAF1 binding |
| ADAP1 | chr7 | 982891 | 982891 | G > A | Change of ZNF227 binding |
| ADAP1 | chr7 | 982891 | 982891 | G > A | Change of ZNF263 binding |
| ADAP1 | chr7 | 982891 | 982891 | G > A | Change of ZNF658B binding |
| ADAP1 | chr7 | 983223 | 983223 | G > C | Change of ATF5 binding |
| ADAP1 | chr7 | 983223 | 983223 | G > C | Change of BCL11A binding |
| ADAP1 | chr7 | 983223 | 983223 | G > C | Change of HOXC5 binding |
| ADAP1 | chr7 | 983223 | 983223 | G > C | Change of SPI1 binding |
| ADAP1 | chr7 | 983223 | 983223 | G > C | Change of ZNF227 binding |
| ADAP1 | chr7 | 983223 | 983223 | G > C | Change of ZNF263 binding |
| ADCY5 | chr3 | 123065778 | 123065778 | A > G | Increasing of enhancer |
| | | | | | activity |
| ADGRL3 | chr4 | 62861941 | 62861941 | G > C | Gain of HRE motif; Loss of |
| | | | | | GRHL1 and TCFCP2L1 motif; |
| | | | | | Decreasing of enhancer |
| | | | | | activity |
| ADGRL3 | chr4 | 62862251 | 62862251 | G > A | Gain of LHX3; BRN3; POU6F2; |
| | | | | | BRN2; OCT1; PIT1 and OC2 |
| | | | | | motif; Loss of PAX2; YYl and |
| | | | | | CRX motif; Decreasing of |
| | | | | | enhancer activity |
| ADGRL3 | chr4 | 62861964 | 62861964 | A > T | Gain of TAXCREB motif; |
| | | | | | Decreasing of enhancer |
| | | | | | activity |
| ADH4 | chr4 | 100078751 | 100078751 | T > G | Loss of enhancer activity |
| ADH4 | chr4 | 100078890 | 100078890 | G > T | Loss of enhancer activity |
| ADO | chr10 | 64445564 | 64445564 | A > G | Increasing of enhancer |
| | | | | | activity |
| ADO | chr10 | 64445564 | 64445564 | A > G | Increasing of enhancer |
| | | | | | activity |
| ADO | chr10 | 64445564 | 64445564 | A > G | Increasing of enhancer |
| | | | | | activity |
| ADO | chr10 | 64445564 | 64445564 | A > G | Increasing of enhancer |
| | | | | | activity |
| ADO | chr10 | 64445564 | 64445564 | A > G | Increasing of enhancer |
| | | | | | activity |
| ADO | chr10 | 64445564 | 64445564 | A > G | Increasing of enhancer |
| | | | | | activity |
| ADSL | chr22 | 40742514 | 40742514 | T > C | Decreasing of enhancer |
| | | | | | activity |
| AFP | chr4 | 74301817 | 74301817 | G > A | Gain of GMEB1 binding; |
| | | | | | Increasing of enhancer |
| | | | | | activity |
| AGAP7P | chr10 | 51549496 | 51549496 | | |
| AGAP7P | chr10 | 51543344 | 51543344 | C > T | Loss of HOXB-like binding; |
| | | | | | Gain of enhancer activity |
| AKT1 | chr14 | 105217554 | 105217554 | G > C | Change of CIC binding |
| AKT1 | chr14 | 105217554 | 105217554 | G > C | Change of NFYA binding |
| AKT1 | chr14 | 105222648 | 105222648 | G > A | Change of ZAC binding |
| AKT1 | chr14 | 105269669 | 105269669 | C > T | Change of ZNF441 binding |
| AKT1 | chr14 | 105444532 | 105444532 | T > G | Change of ZNF579 binding |
| AKT1 | chr14 | 105246325 | 105246325 | T > A | Gain of YY1 binding; Gain of |
| | | | | | enhancer |
| ALAS2 | chrX | 55057617 | 55057617 | G > C | Decreasing of enhancer |
| | | | | | activity |
| ALDOB | chr9 | 104198194 | 104198194 | C > T | Decreasing of enhancer |
| | | | | | activity |
| ALPL | chr1 | 21660786 | 21660786 | C > G | Decreasing of enhancer |
| | | | | | activity |
| ALPL | chr1 | 21661555 | 21661555 | T > G | Decreasing of enhancer |
| | | | | | activity |
| ALPL | chr1 | 21661745 | 21661745 | C > G | Decreasing of enhancer |
| | | | | | activity |
| ALPL | chr1 | 21661878 | 21661878 | C > T | Decreasing of enhancer |
| | | | | | activity |
| ANKEF1 | chr20 | 10016207 | 10016207 | C > G | Loss of GATA2 motif; Loss of |
| | | | | | enhancer activity |
| ANKRD26 | chr10 | 27389383 | 27389383 | C > T | Gain of KLF3;GMEB1; REL; |
| | | | | | SIX2 binding; Increasing of |
| | | | | | enhancer activity |
| ANRIL | chr9 | 22124477 | 22124477 | A > G | Loss of STAT1 motif; |
| | | | | | Decreasing of enhancer |
| | | | | | activity |
| ANRIL | chr9 | 22119195 | 22119195 | | Gain of enhancer activity |
| ANRIL | chr9 | 22118102 | 22118102 | T > G | Increasing of TCF7L2 and |
| | | | | | EP300 binding; Increasing of |
| | | | | | enhancer activity |
| ARFGAP1 | chr20 | 62116270 | 62116270 | A > G | Increasing of enhancer |
| | | | | | activity |
| ARFGAP1 | chr20 | 62116557 | 62116557 | C > T | Increasing of enhancer |
| | | | | | activity |
| ARFGAP1 | chr20 | 62116577 | 62116577 | C > T | Increasing of enhancer |
| | | | | | activity |
| ARFGAP1 | chr20 | 62116662 | 62116662 | A > C | Increasing of enhancer |
| | | | | | activity |
| ARFGAP1 | chr20 | 62116771 | 62116771 | C > T | Increasing of enhancer |
| | | | | | activity |
| ARFGAP1 | chr20 | 62116885 | 62116885 | G > T | Increasing of enhancer |
| | | | | | activity |
| ARFGAP1 | chr20 | 62117295 | 62117295 | T > A | Increasing of enhancer |
| | | | | | activity |
| ARFGAP1 | chr20 | 62117683 | 62117683 | G > T | Increasing of enhancer |
| | | | | | activity |
| ARFGAP1 | chr20 | 62117868 | 62117868 | C > T | Increasing of enhancer |
| | | | | | activity |
| ARFGAP1 | chr20 | 62118212 | 62118212 | C > T | Increasing of enhancer |
| | | | | | activity |
| ARFGAP1 | chr20 | 62118213 | 62118213 | C > T | Increasing of enhancer |
| | | | | | activity |
| ARFGAP1 | chr20 | 62118250 | 62118250 | C > T | Increasing of enhancer |
| | | | | | activity |
| ARFGAP1 | chr20 | 62118282 | 62118282 | A > G | Increasing of enhancer |
| | | | | | activity |
| ARFGAP1 | chr20 | 62118486 | 62118486 | C > T | Increasing of enhancer |
| | | | | | activity |
| ARFGAP1 | chr20 | 62119030 | 62119030 | G > T | Increasing of enhancer |
| | | | | | activity |
| ARFGAP1 | chr20 | 62119047 | 62119047 | C > A | Increasing of enhancer |
| | | | | | activity |
| ARID1A | chr1 | 26438008 | 26438008 | G > A | Change of EBF1 binding |
| ARID1A | chr1 | 26438008 | 26438008 | G > A | Change of HINFP binding |
| ARID1A | chr1 | 26438008 | 26438008 | G > A | Change of SP4 binding |
| ARID1A | chr1 | 26438008 | 26438008 | G > A | Change of TFAP2E binding |
| ARID1A | chr1 | 26438008 | 26438008 | G > A | Change of ZFX binding |
| ARID1A | chr1 | 26798607 | 26798607 | G > A | Change of ELF2 binding |
| ARID1A | chr1 | 26798607 | 26798607 | G > A | Change of EP300 binding |
| ARID1A | chr1 | 26798607 | 26798607 | G > A | Change of TAF1 binding |
| ARID1A | chr1 | 26798607 | 26798607 | G > A | Change of ZBTB7B binding |
| ARID1A | chr1 | 26798607 | 26798607 | G > A | Change of ZNF181 binding |
| ARID1A | chr1 | 26798607 | 26798607 | G > A | Change of ZNF263 binding |
| ARID1A | chr1 | 26798607 | 26798607 | G > A | Change of ZNF383 binding |
| ARID1A | chr1 | 26798607 | 26798607 | G > A | Change of ZNF436 binding |
| ARID1A | chr1 | 26798607 | 26798607 | G > A | Change of ZNF691 binding |
| ARID1A | chr1 | 26798607 | 26798607 | G > A | Change of ZSCAN2 binding |
| ARID1A | chr1 | 26798742 | 26798742 | A > C | Change of HEYL binding |
| ARID1A | chr1 | 26798742 | 26798742 | A > C | Change of ZNF521 binding |
| ARID1A | chr1 | 26798742 | 26798742 | A > C | Change of ZNF546 binding |
| ARID1A | chr1 | 27098601 | 27098601 | G > A | Change of EZF2 binding |
| ARID1A | chr1 | 27098601 | 27098601 | G > A | Change of ZNF729 binding |
| ARID1A | chr1 | 27098601 | 27098601 | G > A | Change of ZNF853 binding |
| ARID1A | chr1 | 27492915 | 27492915 | C > T | Change of APAK binding |
| ARID1A | chr1 | 27492915 | 27492915 | C > T | Change of MZF1 binding |
| ARID1A | chr1 | 27492915 | 27492915 | C > T | Change of ZIM2 binding |
| ARID1A | chr1 | 27492915 | 27492915 | C > T | Change of ZNF407 binding |
| ARID1A | chr1 | 27492915 | 27492915 | C > T | Change of ZNF493 binding |
| ARID1A | chr1 | 27492915 | 27492915 | C > T | Change of ZNF530 binding |
| ARID1A | chr1 | 27492915 | 27492915 | C > T | Change of ZNF816A binding |
| ARID1A | chr1 | 27720081 | 27720081 | G > A | Change of GATA2 binding |
| ARID1A | chr1 | 27720081 | 27720081 | G > A | Change of ZNF814 binding |
| ARID1B | chr6 | 156984421 | 156984421 | A > T | Change of FUBP1 binding |
| ARID1B | chr6 | 157041328 | 157041328 | G > A | Change of SP4 binding |
| ARID1B | chr6 | 157041328 | 157041328 | G > A | Change of TFAP2E binding |
| ARID1B | chr6 | 157041328 | 157041328 | G > A | Change of ZFX binding |
| ARID1B | chr6 | 157110896 | 157110896 | G > A | Change of RXRB binding |
| ARID1B | chr6 | 157110896 | 157110896 | G > A | Change of THRA binding |
| ARID1B | chr6 | 157110896 | 157110896 | G > A | Change of THRB binding |
| ARID1B | chr6 | 157110896 | 157110896 | G > A | Change of ZNF583 binding |
| ARID1B | chr6 | 157154547 | 157154547 | C > T | Change of ZNF492 binding |
| ARID1B | chr6 | 157154547 | 157154547 | C > T | Change of ZNF570 binding |
| ARID1B | chr6 | 157154547 | 157154547 | C > T | Change of ZNF729 binding |
| ARID1B | chr6 | 157389974 | 157389974 | A > G | Change of POU1F1 binding |
| ARLTS1 | chr13 | 50945011 | 50945011 | G > A | Loss of CDC5 motif; Gain of |
| | | | | | PLZF motif; Decreasing of |
| | | | | | enhancer activity |
| ASMT | chrX | 1733783 | 1733783 | A > G | Gain of FOXH1 binding; |
| | | | | | Decreasing of enhancer |
| | | | | | activity |
| ASMT | chrX | 1733892 | 1733892 | C > G | Gain of MAFG; NRL; HEY2 |
| | | | | | binding; Decreasing of |
| | | | | | enhancer activity |
| ATAD3B | chr1 | 928703 | 928703 | C > G | Increasing of enhancer |
| | | | | | activity |
| ATAD3B | chr1 | 930053 | 930053 | G > A | Increasing of enhancer |
| | | | | | activity |
| ATAD3B | chr1 | 930797 | 930797 | G > C | Increasing of enhancer |
| | | | | | activity |
| ATAD3B | chr1 | 931772 | 931772 | G > A | Increasing of enhancer |
| | | | | | activity |
| ATM | chr11 | 107880536 | 107880536 | C > G | Change of HOXC5 binding |
| ATM | chr11 | 108211845 | 108211845 | C > G | Change of ZNF263 binding |
| ATM | chr11 | 108211845 | 108211845 | C > G | Change of ZNF519 binding |
| ATM | chr11 | 108211845 | 108211845 | C > G | Change of ZNF658B binding |
| ATM | chr11 | 109962688 | 109962688 | G > T | Change of ZXDL binding |
| ATP7IP | chr12 | 14410634 | 14410634 | C > T | Decreasing of enhancer |
| | | | | | activity |
| BATF | chr14 | 76005557 | 76005557 | T > C | |
| BCAS3, TBX2, | chr17 | 59456589 | 59456589 | C > C | Increasing of enhancer |
| TBX4 | | | | | activity |
| BCL11A | chr2 | 60718043 | 60718043 | G > T | |
| BCL11A | chr2 | 60718043 | 60718043 | G > T | Loss of GATAl motif; |
| | | | | | Decreasing of enhancer |
| | | | | | activity |
| BCL11A | chr2 | 60725451 | 60725451 | | |
| BCL2, PYGO1 | chr15 | 54197663 | 54203357 | Deletion | Loss of enhancer |
| BCL2, PYGO1 | chr15 | 54197663 | 54203357 | Deletion | Loss of enhancer |
| BCL6 | NA | NA | NA | NA | Gain of enhancer |
| BCL6 | NA | NA | NA | Duplication | Gain of enhancer |
| BCL6 | NA | NA | NA | NA | Gain of enhancer |
| BCL6 | chr3 | 187779884 | 187823100 | Duplication | Gain of enhancer |
| BCL6 | chr3 | 187779884 | 187823100 | Duplication | Gain of enhancer |
| BCL6 | chr3 | 187779884 | 187823100 | Duplication | Gain of enhancer |
| BCL6 | chr3 | 187779884 | 187823100 | Duplication | Gain of enhancer |
| BCL6 | chr3 | 187779884 | 187823100 | Duplication | Gain of enhancer |
| BCL6 | chr3 | 187779884 | 187823100 | Duplication | Gain of enhancer |
| BCL6 | chr3 | 187779884 | 187823100 | Duplication | Gain of enhancer |
| BCL6 | chr3 | 187779884 | 187823100 | Duplication | Gain of enhancer |
| BCL6 | chr3 | 187779884 | 187823100 | Duplication | Gain of enhancer |
| BCL6 | chr3 | 187779884 | 187823100 | Duplication | Gain of enhancer |
| BCL6 | chr3 | 187779884 | 187823100 | Duplication | Gain of enhancer |
| BCL6 | chr3 | 187779884 | 187823100 | Duplication | Gain of enhancer |
| BCL6 | chr3 | 187779884 | 187823100 | Duplication | Gain of enhancer |
| BCL6 | chr3 | 187779884 | 187823100 | Duplication | Gain of enhancer |
| BCL6 | chr3 | 187779884 | 187823100 | Duplication | Gain of enhancer |
| BCL6 | chr3 | 187779884 | 187823100 | Duplication | Gain of enhancer |
| BHLHE41 | chr12 | 26472562 | 26472562 | G > A | Gain of AP-1 motif; Gain of |
| | | | | | enhancer activity |
| BMF | chr15 | 40397936 | 40397936 | C > A | Loss of RELA motif; |
| | | | | | Decreasing of enhancer |
| | | | | | activity |
| BMP2 | chr20 | 7106289 | 7106289 | T > C | Increasing of enhancer |
| | | | | | activity |
| BMP4 | chr14 | 54410919 | 54410919 | T > C | Increasing of enhancer |
| | | | | | activity |
| BRAF | chr7 | 139695303 | 139695303 | G > A | Change of EGR1 binding |
| BRAF | chr7 | 139695303 | 139695303 | G > A | Change of ZNF791 binding |
| BRAF | chr7 | 140845142 | 140845142 | C > A | Change of ZNF236 binding |
| BRAP, | chr12 | 112007756 | 112007756 | C > C | Increasing of enhancer |
| ACAD10, | | | | | activity |
| ATXN2, | | | | | |
| C12orf47, | | | | | |
| ERP29, | | | | | |
| ALDH2, | | | | | |
| MAPKAPK5, | | | | | |
| FAM109A, | | | | | |
| SH2B3, | | | | | |
| TRAFD1 | | | | | |
| BRCA1 | chr17 | 41278377 | 41278377 | G > A | Gain of GMEB1; CEBPE |
| | | | | | binding; Increasing of |
| | | | | | enhancer activity |
| BRCA1 | chr17 | 41445837 | 41445837 | C > G | Change of HINFP binding |
| BRCA1 | chr17 | 41445837 | 41445837 | C > G | Change of YYl binding |
| BRCA2 | chr13 | 32895005 | 32895005 | T > A | Change of ZNF551 binding |
| BRCA2 | chr13 | 33213838 | 33213838 | G > T | Change of CTCF binding |
| BRCA2 | chr13 | 33213838 | 33213838 | G > T | Change of RAD21 binding |
| BRCA2 | chr13 | 33213838 | 33213838 | G > T | Change of SMC3 binding |
| BRCA2 | chr13 | 33213838 | 33213838 | G > T | Change of TAL2 binding |
| BRCA2 | chr13 | 33213838 | 33213838 | G > T | Change of ZNF257 binding |
| BRCA2 | chr13 | 33213838 | 33213838 | G > T | Change of ZNF430 binding |
| BRIP1 | chr17 | 59366414 | 59366414 | C > T | Change of ZNF513 binding |
| BRIP1 | chr17 | 59366414 | 59366414 | C > T | Change of ZNF517 binding |
| BRIP1 | chr17 | 59366943 | 59366943 | A > C | Change of ZNF404 binding |
| BRIP1 | chr17 | 59376306 | 59376306 | C > G | Change of NKX2-4 binding |
| BRIP1 | chr17 | 59376306 | 59376306 | C > G | Change of PBX3 binding |
| BRIP1 | chr17 | 59376306 | 59376306 | C > G | Change of POLR3A binding |
| BRIP1 | chr17 | 59377191 | 59377191 | T > C | Change of FOXB2 binding |
| BRIP1 | chr17 | 59377191 | 59377191 | T > C | Change of NANC > G binding |
| BRIP1 | chr17 | 59377191 | 59377191 | T > C | Change of SOX9 binding |
| BRIP1 | chr17 | 59525138 | 59525138 | G > T | Change of HES4 binding |
| BRIP1 | chr17 | 59525138 | 59525138 | G > T | Change of ZNF627 binding |
| BRIP1 | chr17 | 59715852 | 59715852 | G > C | Change of CEBPA binding |
| BRIP1 | chr17 | 59722479 | 59722479 | T > C | Change of HDAC2 binding |
| BRIP1 | chr17 | 59722479 | 59722479 | T > C | Change of HOXC5 binding |
| BRIP1 | chr17 | 59722479 | 59722479 | T > C | Change of ZNF263 binding |
| BRIP1 | chr17 | 59722480 | 59722480 | C > T | Change of HDAC2 binding |
| BRIP1 | chr17 | 59722480 | 59722480 | C > T | Change of HOXC5 binding |
| BRIP1 | chr17 | 59722480 | 59722480 | C > T | Change of ZNF263 binding |
| BRIP1 | chr17 | 59789086 | 59789086 | A > C | Change of ZNF425 binding |
| BRIP1 | chr20 | 55684882 | 55684882 | G > C | Change of CT53 binding |
| BRIP1 | chr20 | 55684882 | 55684882 | G > C | Change of KLF15 binding |
| BRIP1 | chr20 | 55684882 | 55684882 | G > C | Change of TFAP2A binding |
| BRIP1 | chr20 | 55684882 | 55684882 | G > C | Change of TFAP2E binding |
| BRIP1 | chr20 | 55684882 | 55684882 | G > C | Change of ZNF219 binding |
| BRIP1 | chr20 | 55684882 | 55684882 | G > C | Change of ZNF263 binding |
| BRIP1 | chr20 | 55684882 | 55684882 | G > C | Change of ZNF281 binding |
| BRIP1 | chr20 | 55684882 | 55684882 | G > C | Change of ZNF284 binding |
| BRIP1 | chr20 | 55684882 | 55684882 | G > C | Change of ZNF716 binding |
| BRIP1 | chr20 | 55701207 | 55701207 | C > T | Change of HDAC2 binding |
| BRIP1 | chr20 | 55701207 | 55701207 | C > T | Change of IRF4 binding |
| BRIP1 | chr20 | 55702731 | 55702731 | G > T | Change of PBX4 binding |
| BRIP1 | chr20 | 55774343 | 55774343 | C > G | Change of THAP1 binding |
| BRIP1 | chr20 | 55774343 | 55774343 | C > G | Change of YYl binding |
| BRIP1 | chr20 | 55795637 | 55795637 | G > A | Change of BCL6 binding |
| BRIP1 | chr20 | 55829393 | 55829393 | C > G | Change of HOXB2 binding |
| BRIP1 | chr20 | 55829393 | 55829393 | C > G | Change of HOXB3 binding |
| BRIP1 | chr20 | 55829393 | 55829393 | C > G | Change of ZNF605 binding |
| BTD | | NA | NA | | Gain of enhancer |
| BTD | | NA | NA | | Gain of enhancer |
| BTD | | NA | NA | | Gain of enhancer |
| BTD | | NA | NA | | Gain of enhancer |
| BTD | | NA | NA | | Gain of enhancer |
| BTD | | NA | NA | | Gain of enhancer |
| BTD | | NA | NA | | Gain of enhancer |
| BTD | | NA | NA | | Gain of enhancer |
| BTD | | NA | NA | | Gain of enhancer |
| BTD | | NA | NA | | Gain of enhancer |
| BTD | | NA | NA | | Gain of enhancer |
| BTD | | NA | NA | | Gain of enhancer |
| BTD | | NA | NA | | Gain of enhancer |
| BTN3A2 | chr6 | 28234597 | 28234597 | C > T | Decreasing of enhancer |
| | | | | | activity |
| BTN3A2 | chr6 | 28235176 | 28235176 | A > C | Decreasing of enhancer |
| | | | | | activity |
| C10orf110, | chr10 | 1156165 | 1156165 | T > T | Increasing of enhancer |
| WDR37, IDI2, | | | | | activity |
| PITRM1, | | | | | |
| GTPBP4, | | | | | |
| LARP4B, | | | | | |
| IDI1, PFKP | | | | | |
| C13orf34, | chr13 | 72347696 | 72347696 | A > A | Increasing of enhancer |
| DACH1 | | | | | activity |
| C16orf93 | chr16 | 30669784 | 30669784 | G > C | Change of SP3 binding |
| C16orf93 | chr16 | 30753142 | 30753142 | C > T | Change of SP1 binding |
| C16orf93 | chr16 | 30753142 | 30753142 | C > T | Change of SP3 binding |
| C16orf93 | chr16 | 31153547 | 31153547 | A > C | Change of SP1 binding |
| C16orf93 | chr16 | 31153547 | 31153547 | A > C | Change of SP3 binding |
| C16orf93 | chr16 | 31153547 | 31153547 | A > C | Change of SP1 binding |
| C16orf93 | chr16 | 31153547 | 31153547 | A > C | Change of SP3 binding |
| C16orf93, | chr16 | 30935409 | 30935409 | C > G | Change of SP1 binding |
| PHKG2 | | | | | |
| C16orf93, | chr16 | 30669784 | 30669784 | G > C | Change of SP1 binding |
| SRCAP | | | | | |
| C16orf93, | chr16 | 30669878 | 30669878 | C > T | Change of SP1 binding |
| SRCAP | | | | | |
| C16orf93, | chr16 | 30669784 | 30669784 | G > C | Change of SP1 binding |
| SRCAP, | | | | | |
| PHKG2 | | | | | |
| C16orf93, | chr16 | 30753142 | 30753142 | C > T | Change of SP1 binding |
| SRCAP, | | | | | |
| PHKG2 | | | | | |
| C16orf93, | chr16 | 31153547 | 31153547 | A > C | Change of SP1 binding |
| SRCAP, | | | | | |
| PHKG2 | | | | | |
| C19orf40, | chr19 | 33356891 | 33356891 | T > C | Increasing of enhancer |
| SLC7A10, | | | | | activity |
| CEBPG, | | | | | |
| GPATCH1, | | | | | |
| TDRD12, | | | | | |
| SLC7A9, | | | | | |
| ANKRD27, | | | | | |
| NUDT19, | | | | | |
| CCDC123, | | | | | |
| PDCD5 | | | | | |
| C2orf43 | chr2 | 20882056 | 20882056 | | |
| C5orf35 | chr5 | 56034207 | 56034207 | A > G | |
| CACNA1C | chr12 | 2346830 | 2346830 | T > A | Decreasing of enhancer |
| | | | | | activity |
| CACNA1C | chr12 | 2346393 | 2346393 | C > T | Decreasing of enhancer |
| | | | | | activity |
| CACNA1C | chr12 | 2349584 | 2349584 | G > C | Decreasing of enhancer |
| | | | | | activity |
| CACNA1C | chr12 | 2361460 | 2361460 | C > T | Decreasing of enhancer |
| | | | | | activity |
| CACNA1C | chr12 | 2349584 | 2349584 | G > C | Decreasing of enhancer |
| | | | | | activity |
| CALR | chr19 | 13049218 | 13049218 | C > T | |
| CALR | chr19 | 13049375 | 13049375 | G > C | Gain of GMEB1 binding; |
| | | | | | Increasing of enhancer |
| | | | | | activity |
| CAMK1D, | chr10 | 12307894 | 12307894 | C > T | |
| CDC123 | | | | | |
| CAMK1D, | chr10 | 12307894 | 12307894 | C > T | Gain of FOXA1 and FOXA2 |
| CDC123 | | | | | binding; Gain of enhancer |
| | | | | | activity |
| CAPG | chr2 | 85769711 | 85769711 | | |
| CAPG | chr2 | 85780536 | 85780536 | | |
| CAPG | chr2 | 85762209 | 85762209 | | Change of CTCF binding |
| CARLo-5, | chr8 | 128413305 | 128413305 | T > G | Increasing of enhancer |
| MYC | | | | | activity |
| CARLo-5, | chr8 | 128413305 | 128413305 | G > T | Increasing of enhancer |
| MYC | | | | | activity |
| CARLo-5, | chr8 | 128413305 | 128413305 | T > G | Increasing of enhancer |
| MYC | | | | | activity |
| CARLo-5, | chr8 | 128413305 | 128413305 | G > T | Increasing of enhancer |
| MYC | | | | | activity |
| CASP3 | chr4 | 185569801 | 185569801 | T > G | Decreasing of enhancer |
| | | | | | activity |
| CASP8 | chr2 | 202313604 | 202313604 | G > T | Change of MEF2A binding |
| CASP8 | chr2 | 202313604 | 202313604 | G > T | Change of MEF2C binding |
| CASP8 | chr2 | 202900229 | 202900229 | C > A | Change of SP2 binding |
| CASP8 | chr2 | 202204741 | 202204741 | C > T | Loss of MYC binding; Loss of |
| | | | | | enhancer activity |
| CBX7 | chr22 | 39542292 | 39542292 | G > A | Decreasing of enhancer |
| | | | | | activity |
| CBX7 | chr22 | 39542292 | 39542292 | G > A | Loss of ETS1; ETV4 and PAX6 |
| | | | | | motif; Decreasing of |
| | | | | | enhancer activity |
| CCDC142, | chr2 | 73868328 | 73868328 | A > G | Increasing of enhancer |
| SLC4A5, | | | | | activity |
| CCT7, | | | | | |
| STAM BP, | | | | | |
| FBX041, | | | | | |
| ACTG2, | | | | | |
| NAT8B, | | | | | |
| DUSP11, | | | | | |
| NAT8, | | | | | |
| ALMS1, | | | | | |
| TPRKB, | | | | | |
| DGUOK, | | | | | |
| TET3, BOLA3, | | | | | |
| DCTN1, | | | | | |
| TTC31, | | | | | |
| MOBKL1B | | | | | |
| CCL2 | chr17 | 32579788 | 32579788 | A > G | Gain of MSC; MYF6; PKNOX2; |
| | | | | | TGIF2LX; MEIS3; TCF21; |
| | | | | | SMAD4; TGIF1; ATOH1; |
| | | | | | MEIS2; MYOD1; MYC > G; |
| | | | | | PKNOX1; MEIS1 binding; |
| | | | | | Increasing of enhancer |
| | | | | | activity |
| CCL5 | chr17 | 34207780 | 34207780 | C > T | Gain of GATA2 binding; |
| | | | | | Increasing of enhancer |
| | | | | | activity |
| CCND1 | chr11 | 68859692 | 68859692 | G > C | Change of ZNF585B binding |
| CCND1 | chr11 | 68872778 | 68872778 | C > T | Change of HAND2 binding |
| CCND1 | chr11 | 68872778 | 68872778 | C > T | Change of USF1 binding |
| CCND1 | chr11 | 68872778 | 68872778 | C > T | Change of USF2 binding |
| CCND1 | chr11 | 68872949 | 68872949 | C > T | Change of THAP1 binding |
| CCND1 | chr11 | 68872949 | 68872949 | C > T | Change of ZNF254 binding |
| CCND1 | chr11 | 68872949 | 68872949 | C > T | Change of ZNF306 binding |
| CCND1 | chr11 | 68872949 | 68872949 | C > T | Change of ZNF502 binding |
| CCND1 | chr11 | 68881643 | 68881643 | G > T | Change of IRX3 binding |
| CCND1 | chr11 | 68886052 | 68886052 | G > T | Change of ZNF552 binding |
| CCND1 | chr11 | 69051978 | 69051978 | C > G | Change of ZNF585B binding |
| CCND1 | chr11 | 69053154 | 69053154 | G > A | Change of ZNF829 binding |
| CCND1 | chr11 | 69088100 | 69088100 | G > C | Change of HZF12 binding |
| CCND1 | chr11 | 69088100 | 69088100 | G > C | Change of ZNF143 binding |
| CCND1 | chr11 | 69088100 | 69088100 | G > C | Change of ZNF521 binding |
| CCND1 | chr11 | 69191851 | 69191851 | A > C | Change of CREB1 binding |
| CCND1 | chr11 | 69191851 | 69191851 | A > C | Change of KLF14 binding |
| CCND1 | chr11 | 69191851 | 69191851 | A > C | Change of KLF16 binding |
| CCND1 | chr11 | 69191851 | 69191851 | A > C | Change of SP3 binding |
| CCND1 | chr11 | 69191851 | 69191851 | A > C | Change of SP8 binding |
| CCND1 | chr11 | 69243913 | 69243913 | C > T | Change of ZNF454 binding |
| CCND1 | chr11 | 69252451 | 69252451 | G > A | Change of ZNF281 binding |
| CCND1 | chr11 | 69256005 | 69256005 | A > T | Change of HDAC2 binding |
| CCND1 | chr11 | 69256005 | 69256005 | A > T | Change of IRF1 binding |
| CCND1 | chr11 | 69256008 | 69256008 | C > T | Change of IRF1 binding |
| CCND1 | chr11 | 69256008 | 69256008 | C > T | Change of ZNF136 binding |
| CCND1 | chr11 | 69256011 | 69256011 | T > C | Change of IRF1 binding |
| CCND1 | chr11 | 69256011 | 69256011 | T > C | Change of ZNF136 binding |
| CCND1 | chr11 | 69258373 | 69258373 | A > C | Change of CCNT2 binding |
| CCND1 | chr11 | 69258373 | 69258373 | A > C | Change of GATA2 binding |
| CCND1 | chr11 | 69258373 | 69258373 | A > C | Change of SP1 binding |
| CCND1 | chr11 | 69258373 | 69258373 | A > C | Change of SP4 binding |
| CCND1 | chr11 | 69259344 | 69259344 | C > G | Change of CTCF binding |
| CCND1 | chr11 | 69259344 | 69259344 | C > G | Change of RAD21 binding |
| CCND1 | chr11 | 69259344 | 69259344 | C > G | Change of RXRA binding |
| CCND1 | chr11 | 69259344 | 69259344 | C > G | Change of SMC3 binding |
| CCND1 | chr11 | 69279835 | 69279835 | G > C | Change of ZNF394 binding |
| CCND1 | chr11 | 69279835 | 69279835 | G > C | Change of ZNF396 binding |
| CCND1 | chr11 | 69279835 | 69279835 | G > C | Change of ZNF492 binding |
| CCND1 | chr11 | 69279835 | 69279835 | G > C | Change of ZNF729 binding |
| CCND1 | chr11 | 69279835 | 69279835 | G > C | Change of ZNF99 binding |
| CCND1 | chr11 | 69282363 | 69282363 | C > G | Change of NFAT5 binding |
| CCND1 | chr11 | 69282363 | 69282363 | C > G | Change of NFKB1 binding |
| CCND1 | chr11 | 69282363 | 69282363 | C > G | Change of RELA binding |
| CCND1 | chr11 | 69282363 | 69282363 | C > G | Change of REL binding |
| CCND1 | chr11 | 69282363 | 69282363 | C > G | Change of SALL1 binding |
| CCND1 | chr11 | 69282363 | 69282363 | C > G | Change of STAT1 binding |
| CCND1 | chr11 | 69282363 | 69282363 | C > G | Change of ZNF227 binding |
| CCND1 | chr11 | 69282363 | 69282363 | C > G | Change of ZNF383 binding |
| CCND1 | chr11 | 69282964 | 69282964 | C > G | Change of ZNF776 binding |
| CCND1 | chr11 | 69283489 | 69283489 | C > G | Change of ZNF543 binding |
| CCND1 | chr11 | 69283489 | 69283489 | C > G | Change of ZNF708 binding |
| CCND1 | chr11 | 69287640 | 69287640 | C > T | Change of REST binding |
| CCND1 | chr11 | 69287640 | 69287640 | C > T | Change of TCF12 binding |
| CCND1 | chr11 | 69305144 | 69305144 | C > G | Change of ZNF573 binding |
| CCND1 | chr11 | 69305799 | 69305799 | T > C | Change of NANC > G binding |
| CCND1 | chr11 | 69312746 | 69312746 | T > G | Change of RREB1 binding |
| CCND1 | chr11 | 69312746 | 69312746 | T > G | Change of SP3 binding |
| CCND1 | chr11 | 69312746 | 69312746 | T > G | Change of ZNF658B binding |
| CCND1 | chr11 | 69312746 | 69312746 | T > G | Change of ZNF658 binding |
| CCND1 | chr11 | 69312746 | 69312746 | T > G | Change of ZNF781 binding |
| CCND1 | chr11 | 69315935 | 69315935 | G > T | Change of E2F6 binding |
| CCND1 | chr11 | 69315935 | 69315935 | G > T | Change of MAZ binding |
| CCND1 | chr11 | 69315935 | 69315935 | G > T | Change of ZNF143 binding |
| CCND1 | chr11 | 69315935 | 69315935 | G > T | Change of ZNF263 binding |
| CCND1 | chr11 | 69315935 | 69315935 | G > T | Change of ZNF391 binding |
| CCND1 | chr11 | 69317977 | 69317977 | G > C | Change of RLF binding |
| CCND1 | chr11 | 69317977 | 69317977 | G > C | Change of ZNF514 binding |
| CCND1 | chr11 | 69318175 | 69318175 | G > C | Change of ZNF394 binding |
| CCND1 | chr11 | 69318175 | 69318175 | G > C | Change of ZNF396 binding |
| CCND1 | chr11 | 69324989 | 69324989 | C > T | Change of ARNT2 binding |
| CCND1 | chr11 | 69324989 | 69324989 | C > T | Change of EGR1 binding |
| CCND1 | chr11 | 69324989 | 69324989 | C > T | Change of PLAG1 binding |
| CCND1 | chr11 | 69324989 | 69324989 | C > T | Change of SP1 binding |
| CCND1 | chr11 | 69324989 | 69324989 | C > T | Change of ZNF780A binding |
| CCND1 | chr11 | 69330742 | 69330742 | T > G | Change of BARHL1 binding |
| CCND1 | chr11 | 69330742 | 69330742 | T > G | Change of GSX2 binding |
| CCND1 | chr11 | 69330742 | 69330742 | T > G | Change of HOXA13 binding |
| CCND1 | chr11 | 69330742 | 69330742 | T > G | Change of TLX3 binding |
| CCND1 | chr11 | 69332416 | 69332416 | C > T | Change of ZNF69 binding |
| CCND1 | chr11 | 69331642 | 69331642 | C > G | Loss of ELK4 motif; |
| | | | | | Decreasing of enhancer |
| | | | | | activity |
| CCND1 | chr11 | 69331418 | 69331418 | C > T | Loss of USF1 motif and USF2 |
| | | | | | motif; Decreasing of |
| | | | | | enhancer activity |
| CCND1 | chr11 | 69345852 | 69345852 | G > T | Change of MAZ binding |
| CCND1 | chr11 | 69345852 | 69345852 | G > T | Change of ZNF263 binding |
| CCND1 | chr11 | 69345852 | 69345852 | G > T | Change of ZNF513 binding |
| CCND1 | chr11 | 69351959 | 69351959 | C > G | Change of ZBTB41 binding |
| CCND1 | chr11 | 69355586 | 69355586 | A > C | Change of CTCFL binding |
| CCND1 | chr11 | 69355586 | 69355586 | A > C | Change of GLI1 binding |
| CCND1 | chr11 | 69355586 | 69355586 | A > C | Change of GLI3 binding |
| CCND1 | chr11 | 69390451 | 69390451 | T > A | Change of ATF5 binding |
| CCND1 | chr11 | 69390451 | 69390451 | T > A | Change of IRF4 binding |
| CCND1 | chr11 | 69390451 | 69390451 | T > A | Change of PRDM1 binding |
| CCND1 | chr11 | 69390451 | 69390451 | T > A | Change of ZNF227 binding |
| CCND1 | chr11 | 69390451 | 69390451 | T > A | Change of ZNF263 binding |
| CCND1 | chr11 | 69390451 | 69390451 | T > A | Change of ZNF293 binding |
| CCND1 | chr11 | 69416853 | 69416853 | T > A | Change of PLAG1 binding |
| CCND1 | chr11 | 69418111 | 69418111 | G > C | Change of ZNF320 binding |
| CCND1 | chr11 | 69418111 | 69418111 | G > C | Change of ZNF502 binding |
| CCND1 | chr11 | 69431690 | 69431690 | G > A | Change of ZNF799 binding |
| CCND1 | chr11 | 69465198 | 69465198 | A > T | Change of ZNF836 binding |
| CCND1 | chr11 | 69239741 | 69239741 | G > A | Loss of HIF motif; Decreasing |
| | | | | | of enhancer activity |
| CCND1 | chr11 | 69251982 | 69251982 | C > T | Loss of HIF motif; Decreasing |
| | | | | | of enhancer activity |
| CCND1 | chr11 | 69234620 | 69234620 | T > C | Loss of HIF motif; Decreasing |
| | | | | | of enhancer activity |
| CCNE1 | chr19 | 30296853 | 30296853 | T > C | Gain of KLF5 binding; Gain of |
| | | | | | enhancer |
| CCN1, | chr4 | 77368847 | 77368847 | G > A | Increasing of enhancer |
| NUP54, | | | | | activity |
| STBD1, | | | | | |
| SDAD1, | | | | | |
| CXCL10, | | | | | |
| CCNG2, | | | | | |
| SCARB2, | | | | | |
| SHROOM3, | | | | | |
| CXCL9 | | | | | |
| CCN1, | chr4 | 77412140 | 77412140 | G > A | Increasing of enhancer |
| SCARB2, | | | | | activity |
| STBD1, | | | | | |
| CCNG2, | | | | | |
| NUP54, | | | | | |
| SHROOM3, | | | | | |
| CXCL10, | | | | | |
| SDAD1, | | | | | |
| CXCL9 | | | | | |
| CCR5 | chr3 | 46410171 | 46410171 | C > T | Loss of CREB1 motif; |
| | | | | | Decreasing of enhancer |
| | | | | | activity |
| CD207 | chr2 | 71079245 | 71079245 | C > A | Loss of Ik-2 and SIX5 motif |
| CD40 | chr20 | 44740196 | 44740196 | T > C | Decreasing of enhancer |
| | | | | | activity |
| CD40 | chr20 | 44740196 | 44740196 | T > C | Decreasing of enhancer |
| | | | | | activity |
| CD40 | chr20 | 44740196 | 44740196 | T > C | Decreasing of enhancer |
| | | | | | activity |
| CD40 | chr20 | 44740196 | 44740196 | T > C | Decreasing of enhancer |
| | | | | | activity |
| CD40 | chr20 | 44740196 | 44740196 | T > C | Decreasing of enhancer |
| | | | | | activity |
| CD40 | chr20 | 44740196 | 44740196 | T > C | Decreasing of enhancer |
| | | | | | activity |
| CD58 | chr1 | 117100957 | 117100957 | A > G | |
| CD86 | chr3 | 121793187 | 121793187 | C > A | |
| CDCA7L | chr7 | 21938240 | 21938240 | T > G | Increasing of IRF4 binding; |
| | | | | | Increasing of enhancer |
| | | | | | activity |
| CDCA7L | chr7 | 21938240 | 21938240 | A > C | Loss of GATAl; GATA2 and |
| | | | | | GATA5 motif; Decreasing of |
| | | | | | enhancer activity |
| CDH1 | chr16 | 68738472 | 68738472 | C > T | Change of ZNF80 binding |
| CDH1 | chr16 | 68741288 | 68741288 | G > T | Change of BATF binding |
| CDH1 | chr16 | 68741288 | 68741288 | G > T | Change of FOSL1 binding |
| CDH1 | chr16 | 68741288 | 68741288 | G > T | Change of HMGN3 binding |
| CDH1 | chr16 | 68741288 | 68741288 | G > T | Change of JUNB binding |
| CDH1 | chr16 | 68741288 | 68741288 | G > T | Change of MAFA binding |
| CDH1 | chr16 | 68741288 | 68741288 | G > T | Change of MAFK binding |
| CDH1 | chr16 | 68741288 | 68741288 | G > T | Change of NFE2 binding |
| CDH1 | chr16 | 68741288 | 68741288 | G > T | Change of NR3C1 binding |
| CDH1 | chr16 | 68741288 | 68741288 | G > T | Change of SMARCC1 binding |
| CDH1 | chr16 | 68750191 | 68750191 | T > C | Change of SOX7 binding |
| CDH1 | chr16 | 68750191 | 68750191 | T > C | Change of SOX9 binding |
| CDH1 | chr16 | 68752380 | 68752380 | C > G | Change of NR2F1 binding |
| CDH1 | chr16 | 68752380 | 68752380 | C > G | Change of RARG binding |
| CDH1 | chr16 | 68823620 | 68823620 | C > T | Change of ZNF238 binding |
| CDH1 | chr16 | 68826079 | 68826079 | C > T | Change of IKZF1 binding |
| CDH1 | chr16 | 68833932 | 68833932 | T > A | Change of ARID3C binding |
| CDH1 | chr16 | 68833932 | 68833932 | T > A | Change of MEOX2 binding |
| CDKN1B | chr12 | 12876142 | 12876142 | C > T | Change of ZNF253 binding |
| CDKN1B | chr12 | 12876142 | 12876142 | C > T | Change of ZNF785 binding |
| CDKN1B | chr12 | 12957277 | 12957277 | G > T | Change of NFATC1 binding |
| CDKN2A | chr9 | 21975127 | 21975127 | C > T | Decreasing of enhancer |
| | | | | | activity |
| CDKN2A | chr9 | 22123766 | 22123766 | A > C | Gain of IRF4 motif; Increasing |
| | | | | | of enhancer activity |
| CDKN2A | chr9 | 22124472 | 22124472 | C > T | Loss of TEAD3 and TEAD4 |
| | | | | | motif; Decreasing of |
| | | | | | enhancer activity |
| CDKN2A | chr9 | 22072264 | 22072264 | A > G | Loss of GATA motif; |
| | | | | | Decreasing of enhancer |
| | | | | | activity |
| CDKN2A | chr9 | 22076795 | 22076795 | A > G | Loss of TEAD3 and TEAD4 |
| | | | | | motif; Decreasing of |
| | | | | | enhancer activity |
| CDKN2A | chr9 | 22096055 | 22096055 | A > G | Loss of PRC1 motif; |
| | | | | | Decreasing of enhancer |
| | | | | | activity |
| CDKN2A | chr9 | 22106225 | 22106225 | A > G | Loss of Smad3 motif; |
| | | | | | Decreasing of enhancer |
| | | | | | activity |
| CDKN2A | chr9 | 22106731 | 22106731 | T > A | Loss of TEAD3 and TEAD4 |
| | | | | | motif; Decreasing of |
| | | | | | enhancer activity |
| CDKN2A | chr9 | 22106271 | 22106271 | A > G | Loss of GATA motif; |
| | | | | | Decreasing of enhancer |
| | | | | | activity |
| CDKN2A | chr9 | 22119195 | 22119195 | T > C | Loss of Smad3 motif; |
| | | | | | Decreasing of enhancer |
| | | | | | activity |
| CDKN2A | chr9 | 22116220 | 22116220 | T > C | Loss of GATA motif; |
| | | | | | Decreasing of enhancer |
| | | | | | activity |
| CDKN2A | chr9 | 22094330 | 22094330 | A > G | Loss of TEAD3 and TEAD4 |
| | | | | | motif; Decreasing of |
| | | | | | enhancer activity |
| CDKN2A | chr1 | 241012779 | 241012779 | C > T | Loss of Meisl motif; |
| | | | | | Decreasing of enhancer |
| | | | | | activity |
| CDKN2A | chr9 | 22072638 | 22072638 | A > G | Gain of GATA motif; |
| | | | | | Increasing of enhancer |
| | | | | | activity |
| CDKN2A, | chr9 | 22124477 | 22124477 | A > G | Loss of STAT1 motif; |
| CDKN2B | | | | | Decreasing of enhancer |
| | | | | | activity |
| CDKN2B | chr6 | 74592063 | 74602441 | Deletion | Loss of enhancer |
| CDKN2B, | chr9 | 22124472 | 22124472 | C > T | Loss of STAT1 motif; |
| MTAP, | | | | | Decreasing of enhancer |
| CDKN2A, | | | | | activity |
| CDKN2B-AS1 | | | | | |
| CETP | chr16 | 56995796 | 56995796 | G > A | Decreasing of enhancer |
| | | | | | activity |
| CFTR | chr7 | 117256712 | 117256712 | G > A | |
| CHEK2 | chr22 | 29196386 | 29196386 | G > C | Change of SP2 binding |
| CHEK2 | chr22 | 29196386 | 29196386 | G > C | Change of TFAP4 binding |
| CHEK2 | chr22 | 29196386 | 29196386 | G > C | Change of ZNF614 binding |
| CHEK2 | chr22 | 29196404 | 29196404 | G > C | Change of EGR4 binding |
| CHEK2 | chr22 | 29205398 | 29205398 | G > T | Change of SOX1 binding |
| CHEK2 | chr22 | 29208344 | 29208344 | C > G | Change of MAFF binding |
| CHEK2 | chr22 | 29208344 | 29208344 | C > G | Change of MAFK binding |
| CHEK2 | chr22 | 29210328 | 29210328 | C > T | Change of ELF3 binding |
| CHEK2 | chr22 | 29210328 | 29210328 | C > T | Change of ZNF607 binding |
| chrNAl | chr2 | 175629600 | 175629600 | T > C | Gain of GMEB1; ZNF646; |
| | | | | | KLF3; RUNX1; HES5 binding; |
| | | | | | Decreasing of enhancer |
| | | | | | activity |
| CLEC16A | chr16 | 11448517 | 11448517 | G > A | Increasing of enhancer |
| | | | | | activity |
| CLECL1 | chr12 | 9876091 | 9876091 | G > A | |
| CLPTM1L | chr5 | 1326501 | 1326501 | T > C | Increasing of enhancer |
| | | | | | activity |
| CLPTM1L | chr5 | 1326505 | 1326505 | C > G | Increasing of enhancer |
| | | | | | activity |
| CLPTM1L | chr5 | 1326904 | 1326904 | G > C | Increasing of enhancer |
| | | | | | activity |
| CLPTM1L | chr5 | 1326908 | 1326908 | T > A | Increasing of enhancer |
| | | | | | activity |
| CLPTM1L | chr5 | 1326988 | 1326988 | A > G | Increasing of enhancer |
| | | | | | activity |
| CLPTM1L | chr5 | 1327062 | 1327062 | G > A | Increasing of enhancer |
| | | | | | activity |
| CLPTM1L | chr5 | 1327333 | 1327333 | T > G | Increasing of enhancer |
| | | | | | activity |
| CLPTM1L | chr5 | 1327445 | 1327445 | C > G | Increasing of enhancer |
| | | | | | activity |
| CLPTM1L | chr5 | 1327544 | 1327544 | T > C | Increasing of enhancer |
| | | | | | activity |
| CLPTM1L | chr5 | 1327722 | 1327722 | T > C | Increasing of enhancer |
| | | | | | activity |
| CNKSR3, | chr6 | 154986664 | 154986664 | G > G | Increasing of enhancer |
| RBM16, | | | | | activity |
| TFB1M | | | | | |
| CUX1 | chr7 | 101930034 | 101930034 | G > C | Change of SP3 binding |
| CUX1 | chr7 | 101362186 | 101362186 | T > G | Change of SP3 binding |
| CUX1 | chr7 | 101362597 | 101362597 | A > T | Change of SP3 binding |
| CUX1 | chr7 | 101362186 | 101362186 | T > G | Change of SP3 binding |
| CUX1 | chr7 | 101362597 | 101362597 | A > T | Change of SP3 binding |
| CUX1 | chr7 | 101589950 | 101589950 | G > C | Change of SP3 binding |
| CXorf36 | chrx | 45298055 | 45298055 | | |
| CYBB | chrX | 37639262 | 37639262 | A > C | Decreasing of enhancer |
| | | | | | activity |
| CYBB | chrX | 37639264 | 37639264 | T > C | Decreasing of enhancer |
| | | | | | activity |
| CYBB | chrX | 37639267 | 37639267 | C > T | Decreasing of enhancer |
| | | | | | activity |
| CYP27B1 | chr12 | 58133256 | 58133256 | G > A | |
| DACH1 | chr13 | 72428647 | 72428647 | G > T | Decreasing of enhancer |
| | | | | | activity |
| DACH1 | chr13 | 72428647 | 72428647 | G > T | Decreasing of enhancer |
| | | | | | activity |
| DAPK1 | chr9 | 89725269 | 89725269 | T > C | Increasing of enhancer |
| | | | | | activity |
| DCAF7 | chr17 | 61777324 | 61777324 | A > C | Change of SP4 binding |
| DCAF7 | chr17 | 61777552 | 61777552 | G > A | Change of SP4 binding |
| DCAF7 | chr17 | 61513737 | 61513737 | C > T | Change of SP4 binding |
| DCAF7 | chr17 | 61043674 | 61043674 | C > G | Change of SP4 binding |
| DCLK3 | chr3 | 37034946 | 37034946 | G > A | Loss of TFAP4 binding; Gain |
| | | | | | of ETS family binding motif; |
| | | | | | Gain of enhancer |
| DIAPH1 | chr5 | 141014494 | 141014494 | A > G | |
| DIS3 | chr13 | 73925672 | 73925672 | TCT > ACATCTTTTC | Gain of LEF1; TCF7and |
| | | | | TTTGAGGCTTAAAT | TCF7L2 motif; Decreasing of |
| | | | | AGTATC (SEQ ID | enhancer activity |
| | | | | NO: 479) | |
| DLX6, DLX5 | chr7 | 95552064 | 96432064 | Deletion | Loss of enhancer |
| DRD5, | chr4 | 9995182 | 9995182 | G > G | Increasing of enhancer |
| WDR1, | | | | | activity |
| SLC2A9 | | | | | |
| EDN1 | chr6 | 12903957 | 12903957 | A > G | Increasing of enhancer |
| | | | | | activity |
| EDN1 | chr6 | 12903957 | 12903957 | A > G | Increasing of enhancer |
| | | | | | activity |
| EDN1 | chr6 | 12903957 | 12903957 | A > G | Increasing of enhancer |
| | | | | | activity |
| EDN1 | chr6 | 12903957 | 12903957 | A > G | Increasing of enhancer |
| | | | | | activity |
| EDN1 | chr6 | 12903957 | 12903957 | A > G | Increasing of enhancer |
| | | | | | activity |
| EGFR | chr7 | 55102219 | 55102219 | A > T | Change of NPAS3 binding |
| EGFR | chr7 | 55102219 | 55102219 | A > T | Change of RREB1 binding |
| EGFR | chr7 | 55322571 | 55322571 | C > A | Change of ZNF647 binding |
| EGFR | chr7 | 55322596 | 55322596 | G > A | Change of SP1 binding |
| EGFR | chr7 | 55322596 | 55322596 | G > A | Change of SP4 binding |
| EGFR | chr7 | 55322596 | 55322596 | G > A | Change of ZNF189 binding |
| EGFR | chr7 | 55322596 | 55322596 | G > A | Change of ZNF254 binding |
| EGFR | chr7 | 55322596 | 55322596 | G > A | Change of ZNF263 binding |
| EGFR | chr7 | 55322596 | 55322596 | G > A | Change of ZNF647 binding |
| EGLN2 | chr19 | 41365000 | 41385000 | Deletion | Loss of enhancer |
| EGR2 | chr10 | 64191785 | 64202507 | Deletion | Loss of enhancer |
| EGR2 | chr10 | 64737575 | 64737575 | T > A | Gain of EWSR1-FLI1 motif; |
| | | | | | Increasing of EWSR1-FLI1 |
| | | | | | binding; Gain of enhancer |
| ELL2 | chr5 | 95261538 | 95261538 | G > C | Decreasing of enhancer |
| | | | | | activity |
| ERAP2 | chr5 | 96273298 | 96273298 | G > A | |
| ERG | NA | NA | NA | NA | Loss of d ETS; GATA and E- |
| | | | | | BOX motif; Decreasing of |
| | | | | | enhancer activity |
| ESR1 | chr6 | 150465639 | 150465639 | C > A | Change of ZIM2 binding |
| ESR1 | chr6 | 151924498 | 151924498 | T > C | Decreasing of enhancer |
| | | | | | activity |
| ESR1 | chr6 | 151935148 | 151935148 | C > G | Change of ZNF21 binding |
| ESR1 | chr6 | 151937492 | 151937492 | G > A | Change of PAX6 binding |
| ESR1 | chr6 | 151937492 | 151937492 | G > A | Change of RARB binding |
| ESR1 | chr6 | 151937492 | 151937492 | G > A | Change of ZNF789 binding |
| ESR1 | chr6 | 151955192 | 151955192 | A > G | Decreasing of enhancer |
| | | | | | activity |
| ESR1 | chr6 | 151955219 | 151955219 | G > T | Gain of GATA3 motif; |
| | | | | | Decreasing of enhancer |
| | | | | | activity |
| ESR1 | chr6 | 151957875 | 151957875 | G > C | Change of ZNF20 binding |
| ESR1 | chr6 | 151958185 | 151958185 | G > A | Change of NKX3-1 binding |
| ESR1 | chr6 | 151958185 | 151958185 | G > A | Change of POU3F3 binding |
| ESR1 | chr6 | 151961699 | 151961699 | C > T | Change of HDAC2 binding |
| ESR1 | chr6 | 151961699 | 151961699 | C > T | Change of HOXC5 binding |
| ESR1 | chr6 | 151961699 | 151961699 | C > T | Change of ZNF263 binding |
| ESR1 | chr6 | 152024472 | 152024472 | C > G | Decreasing of enhancer |
| | | | | | activity |
| ESR1 | chr6 | 152232725 | 152232725 | G > C | Change of FOS binding |
| ESR1 | chr6 | 152232725 | 152232725 | G > C | Change of TP63 binding |
| ESR1 | chr6 | 152232725 | 152232725 | G > C | Change of TP73 binding |
| ESR1 | chr6 | 152360637 | 152360637 | T > C | Decreasing of enhancer |
| | | | | | activity |
| ESR1 | chr6 | 151953765 | 151953765 | Deletion | Loss of enhancer |
| ESR1, | chr6 | 151958815 | 151958815 | T > C | Decreasing of enhancer |
| RMND1 | | | | | activity |
| ESR1, | chr6 | 152432902 | 152432902 | T > C | Decreasing of enhancer |
| RMND1 | | | | | activity |
| ESR1, | chr6 | 151949582 | 151949582 | A > C | Decreasing of enhancer |
| RMND1, | | | | | activity |
| CCDC170 | | | | | |
| ESR1, | chr6 | 151949312 | 151949312 | G > A | Decreasing of enhancer |
| RMND1, | | | | | activity |
| CCDC170 | | | | | |
| ETS2 | chr21 | 40283733 | 40283733 | C > T | |
| ETV6 | chr12 | 11883580 | 11883580 | T > C | Change of LMO2 binding |
| ETV6 | chr12 | 11899976 | 11899976 | A > G | Change of ZBTB20 binding |
| ETV6 | chr12 | 12038275 | 12038275 | A > C | Change of SRF binding |
| ETV6 | chr12 | 12038275 | 12038275 | A > C | Change of YYl binding |
| ETV6 | chr12 | 12879708 | 12879708 | T > G | Change of POU2F1 binding |
| ETV6 | chr12 | 12009741 | 12009741 | A > G | Decreasing of MAX binding; |
| | | | | | Decreasing of enhancer |
| | | | | | activity |
| EVI1 | NA | NA | NA | inv(3)(q21q26.2) | Gain of enhancer |
| EVI1 | NA | NA | NA | t(3; 3)(q21; q26.2) | Gain of enhancer |
| EVI1 | NA | NA | NA | Inversion | Gain of enhancer |
| F7 | chr13 | 113760095 | 113760095 | T > G | Decreasing of enhancer |
| | | | | | activity |
| F7 | chr13 | 113760101 | 113760101 | C > T | Decreasing of enhancer |
| | | | | | activity |
| F8 | chrX | 154251046 | 154251046 | G > A | Decreasing of enhancer |
| | | | | | activity |
| F9 | chrX | 138612889 | 138612889 | G > A | Gain of FOXH1 binding; |
| | | | | | Decreasing of enhancer |
| | | | | | activity |
| FAM122A, | chr9 | 71434707 | 71434707 | A > A | Increasing of enhancer |
| FXN | | | | | activity |
| FAM135B | | NA | NA | | Gain of enhancer |
| FAM135B | | NA | NA | | Gain of enhancer |
| FAM135B | | NA | NA | | Gain of enhancer |
| FAM135B | | NA | NA | | Gain of enhancer |
| FAM135B | | NA | NA | | Gain of enhancer |
| FAM135B | | NA | NA | | Gain of enhancer |
| FAM135B | | NA | NA | | Gain of enhancer |
| FAM135B | | NA | NA | | Gain of enhancer |
| FAM135B | | NA | NA | | Gain of enhancer |
| FAM167A | chr8 | 11343973 | 11343973 | C > T | Increasing of enhancer |
| | | | | | activity |
| FAM167A | chr8 | 11343973 | 11343973 | C > T | Increasing of enhancer |
| | | | | | activity |
| FAM167A | chr8 | 11343973 | 11343973 | C > T | Increasing of enhancer |
| | | | | | activity |
| FAM167A | chr8 | 11343973 | 11343973 | C > T | Increasing of enhancer |
| | | | | | activity |
| FAM167A | chr8 | 11343973 | 11343973 | C > T | Increasing of enhancer |
| | | | | | activity |
| FAM167A | chr8 | 11343973 | 11343973 | C > T | Increasing of enhancer |
| | | | | | activity |
| FAM84B | chr8 | 127888731 | 127888731 | | |
| FGER2 | chr10 | 123333997 | 123333997 | C > T | Gain of C/EBPbeta motif; |
| | | | | | Increasing of enhancer |
| | | | | | activity |
| FGF10, | chr5 | 44706498 | 44706498 | A > G | Gain of FOXA1;OCT1 bs; Gain |
| MRPS30 | | | | | of enhancer activity |
| FGFR2 | chr10 | 123340311 | 123340311 | T > C | Gain of ER-alpha binding; |
| | | | | | Gain of FOXA1 binding; |
| | | | | | Increasing of enhancer |
| | | | | | activity |
| FGFR2 | chr10 | 123340311 | 123340311 | T > C | Gain of FOXA1 binding; Gain |
| | | | | | of enhancer |
| FGFR2 | chr10 | 123340311 | 123340311 | C > T | Gain of Oct-1/Runx2 motif; |
| | | | | | Increasing of enhancer |
| | | | | | activity |
| FGFR2 | chr10 | 123340432 | 123340432 | - > C | Gain of E2F1 binding; Gain of |
| | | | | | enhancer |
| FGFR2 | chr10 | 123340432 | 123340432 | - > C | Gain of E2F1 binding; |
| | | | | | Increasing of enhancer |
| | | | | | activity |
| FGFR2 | chr10 | 123349324 | 123349324 | T > A | Gain of GATA3 binding; Gain |
| | | | | | of enhancer |
| FGFR3 | chr4 | 1770844 | 1770844 | C > G | Change of MAZ binding |
| FGFR3 | chr4 | 1770844 | 1770844 | C > G | Change of SP4 binding |
| FGFR3 | chr4 | 1771286 | 1771286 | C > T | Change of SP4 binding |
| FGFR3 | chr4 | 1772212 | 1772212 | T > G | Change of SP4 binding |
| FGFR3 | chr4 | 1773418 | 1773418 | A > C | Change of MAZ binding |
| FGFR3 | chr4 | 1773418 | 1773418 | A > C | Change of SP4 binding |
| FGFR3 | chr4 | 1762678 | 1762678 | C > T | Change of MAZ binding |
| FGFR3 | chr4 | 1762678 | 1762678 | C > T | Change of SP4 binding |
| FGFR3 | chr4 | 1805959 | 1805959 | C > G | Change of MAZ binding |
| FN1 | chr2 | 216532560 | 216532560 | G > T | Increasing of enhancer |
| | | | | | activity |
| FN1 | chr2 | 216532635 | 216532635 | G > A | Increasing of enhancer |
| | | | | | activity |
| FN1 | chr2 | 216532985 | 216532985 | A > T | Increasing of enhancer |
| | | | | | activity |
| FN1 | chr2 | 216533394 | 216533394 | G > A | Increasing of enhancer |
| | | | | | activity |
| FOXA1 | chr14 | 37710185 | 37710185 | A > C | Change of ZNF611 binding |
| FOXA1 | chr14 | 37710806 | 37710806 | T > G | Change of CTCFL binding |
| FOXA1 | chr14 | 37710806 | 37710806 | T > G | Change of EGR1 binding |
| FOXA1 | chr14 | 37710806 | 37710806 | T > G | Change of EGR3 binding |
| FOXA1 | chr14 | 37710806 | 37710806 | T > G | Change of EGR4 binding |
| FOXA1 | chr14 | 37710806 | 37710806 | T > G | Change of GIC > T1 binding |
| FOXA1 | chr14 | 37710806 | 37710806 | T > G | Change of KLF15 binding |
| FOXA1 | chr14 | 37710806 | 37710806 | T > G | Change of MAZ binding |
| FOXA1 | chr14 | 37710806 | 37710806 | T > G | Change of NPAS3 binding |
| FOXA1 | chr14 | 37710806 | 37710806 | T > G | Change of RREB1 binding |
| FOXA1 | chr14 | 37710806 | 37710806 | T > G | Change of SP1 binding |
| FOXA1 | chr14 | 37710806 | 37710806 | T > G | Change of SP3 binding |
| FOXA1 | chr14 | 37710806 | 37710806 | T > G | Change of SP4 binding |
| FOXA1 | chr14 | 37710806 | 37710806 | T > G | Change of ZBTB7B binding |
| FOXA1 | chr14 | 37710806 | 37710806 | T > G | Change of ZNF100 binding |
| FOXA1 | chr14 | 37710806 | 37710806 | T > G | Change of ZNF219 binding |
| FOXA1 | chr14 | 37710806 | 37710806 | T > G | Change of ZNF436 binding |
| FOXA1 | chr14 | 37710806 | 37710806 | T > G | Change of ZNF485 binding |
| FOXA1 | chr14 | 37710806 | 37710806 | T > G | Change of ZNF658B binding |
| FOXA1 | chr14 | 37723745 | 37723745 | C > T | Change of EGR4 binding |
| FOXA1 | chr14 | 37723745 | 37723745 | C > T | Change of ZBTB41 binding |
| FOXA1 | chr14 | 37723745 | 37723745 | C > T | Change of ZNF148 binding |
| FOXA1 | chr14 | 37733220 | 37733220 | G > A | Change of MEF2A binding |
| FOXA1 | chr14 | 38058268 | 38058268 | C > G | Change of MECOM binding |
| FOXA1 | chr14 | 38118241 | 38118241 | C > T | Change of ZFPM1 binding |
| FOXA1 | chr14 | 38120099 | 38120099 | G > C | Change of ESR1 binding |
| FOXA1 | chr14 | 38120099 | 38120099 | G > C | Change of ESRRA binding |
| FOXA1 | chr14 | 38120099 | 38120099 | G > C | Change of ESRRB binding |
| FOXA1 | chr14 | 38120099 | 38120099 | G > C | Change of NR4A3 binding |
| FOXA1 | chr14 | 38120099 | 38120099 | G > C | Change of NR5A1 binding |
| FOXA1 | chr14 | 38120099 | 38120099 | G > C | Change of NR5A2 binding |
| FOXA1 | chr14 | 38126241 | 38126241 | C > A | Change of ZFP37 binding |
| FOXA1 | chr14 | 38164005 | 38164005 | G > C | Change of IKZF1 binding |
| FOXA1 | chr14 | 38167448 | 38167448 | C > A | Change of EVX2 binding |
| FOXA1 | chr14 | 38179158 | 38179158 | T > G | Change of HNF1A binding |
| FOXA1 | chr14 | 38182676 | 38182676 | C > T | Change of THRA binding |
| FOXA1 | chr14 | 38182684 | 38182684 | C > T | Change of ZNF226 binding |
| FOXA1 | chr14 | 38182915 | 38182915 | C > G | Change of ZNF211 binding |
| FOXA1 | chr14 | 38184293 | 38184293 | G > C | Change of NHLH2 binding |
| FOXE1 | chr9 | 100549013 | 100549013 | A > G | Gain of E-box motif; Gain of |
| | | | | | enhancer activity |
| FOXE1, | chr9 | 100556972 | 100556972 | G > A | Increasing of TFAP2A binding; |
| PTCSC2 | | | | | Increasing of enhancer |
| | | | | | activity |
| FOXE1, | chr9 | 100551768 | 100551768 | A > G | Increasing of TFAP2A binding; |
| PTCSC2 | | | | | Increasing of enhancer |
| | | | | | activity |
| FOXE1, | chr9 | 100551908 | 100551908 | A > C | Increasing of enhancer |
| PTCSC2 | | | | | activity |
| FOXE1, | chr9 | 100548934 | 100548934 | T > C | Decreasing of CEBPb binding; |
| PTCSC2 | | | | | Diseasing of enhancer activity |
| FOXE1, | chr9 | 100556109 | 100556109 | G > A | Increasing of TFAP2A binding; |
| PTCSC2 | | | | | Increasing of enhancer |
| | | | | | activity |
| FOXG1 | chr14 | 30000405 | 30000405 | G > T | Decreasing of enhancer |
| | | | | | activity |
| FOXL2 | chr3 | 138948982 | 138956382 | Deletion | Loss of enhancer |
| FOXL2 | chr3 | 138949151 | 138956509 | Deletion | Loss of enhancer |
| FOXL2 | chr3 | 138949151 | 138956509 | Deletion | Loss of enhancer |
| FOXL2 | chr3 | 138949151 | 138956509 | Deletion | Loss of enhancer |
| FOXL2 | chr3 | 138949151 | 138956509 | Deletion | Loss of enhancer |
| FOXL2 | chr3 | 138949151 | 138956509 | Deletion | Loss of enhancer |
| FOXL2 | chr3 | 138949151 | 138956509 | Deletion | Loss of enhancer |
| FOXL2 | chr3 | 138949151 | 138956509 | Deletion | Loss of enhancer |
| FOXL2 | chr3 | 138949151 | 138956509 | Deletion | Loss of enhancer |
| FOXL2 | chr3 | 140431841 | 140439199 | Deletion | Loss of enhancer |
| FOXL2 | chr3 | 140431841 | 140439199 | Deletion | Loss of enhancer |
| FOXL2 | chr3 | 140431841 | 140439199 | Deletion | Loss of enhancer |
| FOXL2 | chr3 | 140431841 | 140439199 | Deletion | Loss of enhancer |
| FOXL2 | chr3 | 140431841 | 140439199 | Deletion | Loss of enhancer |
| FOXL2 | chr3 | 140431841 | 140439199 | Deletion | Loss of enhancer |
| FOXL2 | chr3 | 140431841 | 140439199 | Deletion | Loss of enhancer |
| FOXL2 | chr3 | 140431841 | 140439199 | Deletion | Loss of enhancer |
| FRG2 | chr4 | 190959438 | 190959438 | C > T | Change of SP8 binding |
| FRG2 | chr4 | 190959439 | 190959439 | G > A | Change of SP8 binding |
| FRG2 | chr4 | 190959651 | 190959651 | G > C | Change of SP8 binding |
| FTO | chr16 | 53816275 | 53816275 | C > A | |
| FTO | chr16 | 53816752 | 53816752 | A > G | |
| FTO | chr16 | 53816838 | 53816838 | G > A | |
| GAD1 | chr2 | 171690121 | 171690121 | A > G | Decreasing of enhancer |
| | | | | | activity |
| GAD1 | chr2 | 171678379 | 171678379 | A > G | Decreasing of enhancer |
| | | | | | activity |
| GAP43 | chr3 | 115377646 | 115377646 | G > T | Decreasing of enhancer |
| | | | | | activity |
| GATA2 | NA | NA | NA | t(3; 3)(q21; q26.2) | Loss of enhancer |
| GATA2 | NA | NA | NA | inv(3)(q21q26.2) | Loss of enhancer |
| GATA2 | chr3 | 128202137 | 128202161 | | Loss of ETS motif; Decreasing |
| | | | | | of enhancer activity |
| GATA2 | chr3 | 128202154 | 128202154 | C > T | Decreasing of enhancer |
| | | | | | activity |
| GATA3 | chr10 | 6244293 | 6244293 | C > T | Change of MAZ binding |
| GATA3 | chr10 | 6244293 | 6244293 | C > T | Change of ZNF263 binding |
| GATA3 | chr10 | 7567024 | 7567024 | T > A | Change of MSX1 binding |
| GATA3 | chr10 | 7928151 | 7928151 | C > G | Change of ZNF117 binding |
| GATA3 | chr10 | 8067866 | 8067866 | G > C | Change of HOXC5 binding |
| GATA3 | chr10 | 8102520 | 8102520 | T > A | Change of STAT4 binding |
| GATA3 | chr10 | 8102598 | 8102598 | G > T | Change of ESR1 binding |
| GATA3 | chr10 | 8102598 | 8102598 | G > T | Change of SP3 binding |
| GATA3 | chr10 | 8119116 | 8119116 | T > G | Change of FOS binding |
| GATA3 | chr10 | 8374958 | 8374958 | C > T | Change of EGR1 binding |
| GATA3 | chr10 | 8374958 | 8374958 | C > T | Change of MAZ binding |
| GATA3 | chr10 | 8374958 | 8374958 | C > T | Change of PLAG1 binding |
| GATA3 | chr10 | 8374958 | 8374958 | C > T | Change of SP1 binding |
| GATA3 | chr10 | 8374958 | 8374958 | C > T | Change of SP2 binding |
| GATA3 | chr10 | 8374958 | 8374958 | C > T | Change of SP3 binding |
| GATA3 | chr10 | 8374958 | 8374958 | C > T | Change of SP4 binding |
| GATA3 | chr10 | 8374958 | 8374958 | C > T | Change of WT1 binding |
| GATA3 | chr10 | 8374958 | 8374958 | C > T | Change of ZBTB7B binding |
| GATA3 | chr10 | 8374958 | 8374958 | C > T | Change of ZNF148 binding |
| GATA3 | chr10 | 8374958 | 8374958 | C > T | Change of ZNF189 binding |
| GATA3 | chr10 | 8374958 | 8374958 | C > T | Change of ZNF263 binding |
| GATA3 | chr10 | 8374958 | 8374958 | C > T | Change of ZNF281 binding |
| GATA3 | chr10 | 8374958 | 8374958 | C > T | Change of ZNF658B binding |
| GATA3 | chr10 | 8374958 | 8374958 | C > T | Change of ZNF823 binding |
| GATA3 | chr10 | 8393839 | 8393839 | C > A | Change of FOXB2 binding |
| GATA3 | chr10 | 8393839 | 8393839 | C > A | Change of FOXP4 binding |
| GATA3 | chr10 | 8393839 | 8393839 | C > A | Change of KIAA0415 binding |
| GATA3 | chr10 | 8393839 | 8393839 | C > A | Change of ZNF227 binding |
| GATA3 | chr10 | 8402402 | 8402402 | G > A | Change of E4F1 binding |
| GATA3 | chr10 | 8405624 | 8405624 | G > A | Change of ZFP14 binding |
| GATA3 | chr10 | 8405624 | 8405624 | G > A | Change of ZIK1 binding |
| GATA3 | chr10 | 8405624 | 8405624 | G > A | Change of ZNF816A binding |
| GATA3 | chr10 | 8899177 | 8899177 | G > C | Change of TBX3 binding |
| GATA3 | chr10 | 8899177 | 8899177 | G > C | Change of ZNF670 binding |
| GATA3 | chr10 | 8899312 | 8899312 | A > T | Change of HSF1 binding |
| GATA3 | chr10 | 8899312 | 8899312 | A > T | Change of ZNF238 binding |
| GATA3 | chr10 | 8900652 | 8900652 | G > A | Change of STAT5A binding |
| GATA3 | chr10 | 8900652 | 8900652 | G > A | Change of STAT5B binding |
| GATA3 | chr10 | 8903379 | 8903379 | A > T | Change of ZNF331 binding |
| GATA3 | chr10 | 8911062 | 8911062 | C > G | Change of SP4 binding |
| GATA3 | chr10 | 9079498 | 9079498 | G > T | Change of HNF1A binding |
| GATA3 | chr10 | 9079498 | 9079498 | G > T | Change of HNF1B binding |
| GATA3 | chr10 | 9079498 | 9079498 | G > T | Change of ONECUT1 binding |
| GATA3 | chr10 | 9079498 | 9079498 | G > T | Change of ZNF300 binding |
| GATA3 | chr10 | 9112313 | 9112313 | G > C | Change of NPAS3 binding |
| GATA3 | chr10 | 9117103 | 9117103 | G > A | Change of ZNF441 binding |
| GATA3 | chr10 | 9201133 | 9201133 | C > A | Change of PRDM9 binding |
| GATA3 | chr10 | 9215998 | 9215998 | C > G | Change of POU6F2 binding |
| GATA3 | chr10 | 9343954 | 9343954 | C > G | Change of FOS binding |
| GATA3 | chr10 | 9343954 | 9343954 | C > G | Change of FOXB2 binding |
| GATA3 | chr10 | 9343954 | 9343954 | C > G | Change of PBX3 binding |
| GATAD2A | chr19 | 19434350 | 19434350 | C > T | Decreasing of enhancer |
| | | | | | activity |
| GCLM | chr1 | 94375309 | 94375309 | G > A | Decreasing of enhancer |
| | | | | | activity |
| GFI1 | NA | NA | NA | NA | Gain of enhancer |
| GFI1 | NA | NA | NA | NA | Gain of enhancer |
| GFI1 | chr1 | 93009313 | 93009313 | A > G | Increasing of enhancer |
| | | | | | activity |
| GIGYF2 | | NA | NA | | Gain of enhancer |
| GIGYF2 | | NA | NA | | Gain of enhancer |
| GIGYF2 | | NA | NA | | Gain of enhancer |
| GIGYF2 | | NA | NA | | Gain of enhancer |
| GIGYF2 | | NA | NA | | Gain of enhancer |
| GIGYF2 | | NA | NA | | Gain of enhancer |
| GIGYF2 | | NA | NA | | Gain of enhancer |
| GIGYF2 | | NA | NA | | Gain of enhancer |
| GIGYF2 | | NA | NA | | Gain of enhancer |
| GIGYF2 | | NA | NA | | Gain of enhancer |
| GIGYF2 | | NA | NA | | Gain of enhancer |
| GIGYF2 | | NA | NA | | Gain of enhancer |
| GIGYF2 | | NA | NA | | Gain of enhancer |
| GIGYF2 | | NA | NA | | Gain of enhancer |
| GIGYF2 | | NA | NA | | Gain of enhancer |
| GJB1 | chrX | 70443031 | 70443031 | G > C | Gain of GMEB1; KLF3 binding; |
| | | | | | Decreasing of enhancer |
| | | | | | activity |
| GNL3 | chr3 | 52742537 | 52742537 | A > T | Decreasing of enhancer |
| | | | | | activity |
| GREM1 | chr15 | 32993111 | 32993111 | C > T | Increasing of enhancer |
| | | | | | activity |
| GSDMC | chr8 | 129978038 | 129978038 | | |
| GSK3B | chr3 | 119814957 | 119814957 | T > A | Increasing of enhancer |
| | | | | | activity |
| H19 | chr11 | 2020124 | 2020124 | C > A | Gain of SMAD4; HEY1 binding |
| HAPLN4 | chr19 | 19431963 | 19431963 | G > T | Decreasing of enhancer |
| | | | | | activity |
| HBB | chr11 | 5248393 | 5248393 | G > A | Decreasing of enhancer |
| | | | | | activity |
| HBB | chr11 | 5248402 | 5248402 | G > A | Decreasing of enhancer |
| | | | | | activity |
| HBB | chr11 | 5248491 | 5248491 | C > T | Decreasing of enhancer |
| | | | | | activity |
| HBD | chr11 | 5255749 | 5255749 | G > T | Decreasing of enhancer |
| | | | | | activity |
| HBD | chr11 | 5255778 | 5255778 | C > T | Gain of GMEB1 binding; |
| | | | | | Decreasing of enhancer |
| | | | | | activity |
| HBG1 | chr11 | 5271262 | 5271262 | A > G | Gain of ATOH1 binding; |
| | | | | | Increasing of enhancer |
| | | | | | activity |
| HBG1 | chr11 | 5271289 | 5271289 | G > A | Increasing of enhancer |
| | | | | | activity |
| HBG2 | chr11 | 5276120 | 5276120 | C > A | Increasing of enhancer |
| | | | | | activity |
| HBG2 | chr11 | 5276172 | 5276172 | C > T | Increasing of enhancer |
| | | | | | activity |
| HBG2 | chr11 | 5276186 | 5276186 | A > G | Gain of GMEB1; KLF3; ATOH1 |
| | | | | | binding; Increasing of |
| | | | | | enhancer activity |
| HBG2 | chr11 | 5276213 | 5276213 | G > C | Increasing of enhancer |
| | | | | | activity |
| HBM | chr16 | 209709 | 209709 | T > C | Decreasing of enhancer |
| | | | | | activity |
| HERC5, PIGY, | chr4 | 88959922 | 88959922 | G > C | Increasing of enhancer |
| ABCG2, | | | | | activity |
| PPM1K, | | | | | |
| SPARCL1, | | | | | |
| HERC6, PKD2 | | | | | |
| HHIP | chr4 | 145488334 | 145488334 | T > C | Gain of Sp3 motif; Decreasing |
| | | | | | of enhancer activity |
| HHIP | chr4 | 145487934 | 145487934 | G > A | Decreasing of enhancer |
| | | | | | activity |
| HLA-DRB1, | chr6 | 31888367 | 31888367 | A > G | Decreasing of enhancer |
| HLA-C, HLA- | | | | | activity |
| DRB5, HLA- | | | | | |
| DQB1 | | | | | |
| HMBS | chr11 | 118955622 | 118955622 | T > A | Gain of GMEB1; HEY2 |
| | | | | | binding; Decreasing of |
| | | | | | enhancer activity |
| HMBS | chr11 | 118955641 | 118955641 | C > T | Gain of ESRRA; NR5A2 |
| | | | | | binding; Decreasing of |
| | | | | | enhancer activity |
| HMCN1 | chr1 | 185904001 | 185904001 | T > C | Decreasing of enhancer |
| | | | | | activity |
| HMCN1 | chr1 | 185904577 | 185904577 | C > T | Decreasing of enhancer |
| | | | | | activity |
| HNF1A | chr12 | 121416034 | 121416034 | G > C | Decreasing of enhancer |
| | | | | | activity |
| HNF1A | chr12 | 121416448 | 121416448 | G > C | Gain of WT1; EGR1 binding; |
| | | | | | Increasing of enhancer |
| | | | | | activity |
| HNF4A | chr20 | 42984276 | 42984276 | C > T | Decreasing of enhancer |
| | | | | | activity |
| HOTAIR | chr12 | 54360232 | 54360232 | C > T | Increasing of enhancer |
| | | | | | activity |
| HRH2 | chr5 | 175109219 | 175109219 | A > G | |
| HTR2A | chr13 | 47471478 | 47471478 | C > T | |
| HTR3A | chr11 | 113846006 | 113846006 | C > T | |
| HTRA1 | chr10 | 124217205 | 124217259 | | Gain of LYRIC binding; Gain of |
| | | | | | enhancer |
| ICOSLG | chr21 | 45615561 | 45615561 | A > G | Increasing of enhancer |
| | | | | | activity |
| ICOSLG | chr21 | 45615561 | 45615561 | A > G | Increasing of enhancer |
| | | | | | activity |
| ICOSLG | chr21 | 45615561 | 45615561 | A > G | Increasing of enhancer |
| | | | | | activity |
| ICOSLG | chr21 | 45615561 | 45615561 | A > G | Increasing of enhancer |
| | | | | | activity |
| ICOSLG | chr21 | 45615561 | 45615561 | A > G | Increasing of enhancer |
| | | | | | activity |
| ICOSLG | chr21 | 45615561 | 45615561 | A > G | Increasing of enhancer |
| | | | | | activity |
| ID2 | chr2 | 8450123 | 8450123 | A > G | Increasing of enhancer |
| | | | | | activity |
| IGF2BP2 | chr3 | 185514393 | 185514393 | T > C | |
| IGF2BP2 | chr3 | 185515635 | 185515635 | G > A | |
| IGF2BP2 | chr3 | 185513296 | 185513296 | T > A | |
| IGF2BP2 | chr3 | 185514931 | 185514931 | T > C | |
| IGF2BP2 | chr3 | 185513392 | 185513392 | A > G | |
| IGFBP5 | chr2 | 217909463 | 217909463 | G > A | Increasing of enhancer |
| | | | | | activity |
| IHH | chr2 | 219925238 | 219974238 | Duplication | Gain of enhancer |
| IHH | chr2 | 219925238 | 219974238 | Duplication | Gain of enhancer |
| IKZF3 | chr17 | 37912377 | 37912377 | C > T | Decreasing of enhancer |
| | | | | | activity |
| IL10 | chr1 | 206946407 | 206946407 | G > T | Decreasing of enhancer |
| | | | | | activity |
| IL10 | chr1 | 206946634 | 206946634 | A > G | Gain of NRL binding; |
| | | | | | Increasing of enhancer |
| | | | | | activity |
| IL10 | chr1 | 206946862 | 206946862 | C > T | Decreasing of enhancer |
| | | | | | activity |
| IL12B | chr5 | 158759900 | 158759900 | A > G | |
| IL-18 | chr11 | 112026156 | 112026156 | C > G | Gain of enhancer activity |
| IL-1A | chr2 | 113542960 | 113542960 | T > C | |
| IL-1B | chr2 | 113590390 | 113590390 | C > T | |
| IL2RA | chr10 | 6096036 | 6096036 | C > T | Loss of GABPA motif; |
| | | | | | Decreasing of GABPA binding; |
| | | | | | Decreasing of enhancer |
| | | | | | activity |
| IL2RA | chr10 | 6102012 | 6102012 | A > G | Increasing of ER binding; |
| | | | | | Increasing of enhancer |
| | | | | | activity |
| IL2RA | chr10 | 6093139 | 6093139 | G > A | Loss of ELF1 motif; |
| | | | | | Decreasing of ELF1 binding; |
| | | | | | Increasing of RUNX3 binding; |
| | | | | | Decreasing of enhancer |
| | | | | | activity |
| IL2RA | chr10 | 6093140 | 6093140 | C > T | Loss of ELF1 motif; |
| | | | | | Decreasing of ELF1 binding; |
| | | | | | Increasing of RUNX3 binding; |
| | | | | | Decreasing of enhancer |
| | | | | | activity |
| IL2RA | chr10 | 6100790 | 6100790 | G > A | Decreasing of EBF1 binding; |
| | | | | | Decreasing of enhancer |
| | | | | | activity |
| IL2RA | chr10 | 6099045 | 6099045 | A > G | Decreasing of TFAP4 binding; |
| | | | | | Loss of enhancer |
| IL2RA | chr10 | 6094697 | 6094697 | C > T | Decreasing of MEF2C binding; |
| | | | | | Loss of enhancer |
| IL2RA | chr10 | 6102012 | 6102012 | A > G | Gain of ER-alpha binding; |
| | | | | | Gain of enhancer |
| IL2RA | chr10 | 6101713 | 6101713 | T > C | |
| IL2RA | chr10 | 6110829 | 6110829 | C > T | |
| IL2RA | chr10 | 6094697 | 6094697 | C > T | Loss of enhancer |
| IL2RA | chr10 | 6094697 | 6094697 | C > T | Loss of enhancer |
| IL4 | chr5 | 132009154 | 132009154 | C > T | Gain of KLF3 binding; |
| | | | | | Increasing of enhancer |
| | | | | | activity |
| IL6 | chr7 | 22766542 | 22766542 | A > C | Increasing of enhancer |
| | | | | | activity |
| INS | chr11 | 2182419 | 2182419 | T > G | Decreasing of enhancer |
| | | | | | activity |
| INSC | | NA | NA | | Gain of enhancer |
| INSC | | NA | NA | | Gain of enhancer |
| INSC | | NA | NA | | Gain of enhancer |
| INSC | | NA | NA | | Gain of enhancer |
| INSC | | NA | NA | | Gain of enhancer |
| INTS1, | chr7 | 1285195 | 1285195 | A > T | Increasing of enhancer |
| MICALL2 | | | | | activity |
| IRAK3 | chr12 | 66581616 | 66581616 | A > G | Increasing of enhancer |
| | | | | | activity |
| IRF5 | chr7 | 128573967 | 128573967 | G > A | Increasing of enhancer |
| | | | | | activity |
| IRF5 | chr7 | 128573967 | 128573967 | G > A | Increasing of enhancer |
| | | | | | activity |
| IRF5 | chr7 | 128573967 | 128573967 | G > A | Increasing of enhancer |
| | | | | | activity |
| IRF5 | chr7 | 128573967 | 128573967 | G > A | Increasing of enhancer |
| | | | | | activity |
| IRF5 | chr7 | 128573967 | 128573967 | G > A | Increasing of enhancer |
| | | | | | activity |
| IRF5 | chr7 | 128573967 | 128573967 | G > A | Increasing of enhancer |
| | | | | | activity |
| IRF6 | chr1 | 209989270 | 209989270 | G > A | Decreasing of enhancer |
| | | | | | activity |
| IRF6 | chr1 | 209989270 | 209989270 | G > A | Loss of AP-2alpha motif and |
| | | | | | TFAP2A motif; Decreasing of |
| | | | | | enhancer activity |
| IRF6 | chr1 | 209989270 | 209989270 | G > A | Loss of AP-2CE± binding; Loss |
| | | | | | of enhancer activity |
| IRS4 | | NA | NA | | Gain of enhancer |
| IRS4 | | NA | NA | | Gain of enhancer |
| IRS4 | | NA | NA | | Gain of enhancer |
| IRS4 | | NA | NA | | Gain of enhancer |
| IRX3 | chr16 | 53830465 | 53830465 | G > A | Increasing of enhancer |
| | | | | | activity |
| IRX3 | chr16 | 53830465 | 53830465 | G > A | Increasing of enhancer |
| | | | | | activity |
| IRX5, IRX3 | chr16 | 53800954 | 53800954 | T > C | Loss of ARID5B motif; Gain of |
| | | | | | enhancer |
| ITGAM | chr16 | 31276811 | 31276811 | G > A | Decreasing of enhancer |
| | | | | | activity |
| ITK | chr5 | 156607793 | 156607793 | C > T | Increasing of enhancer |
| | | | | | activity |
| JAZF1 | chr7 | 27976563 | 27976563 | A > G | Gain of NKX3-1 motif; Loss of |
| | | | | | FOXA1 motif; Decreasing of |
| | | | | | enhancer activity |
| JMJD2C, | chr16 | 68820946 | 68820946 | G > A | Decreasing of enhancer |
| TMED6 | | | | | activity |
| JUN | chr1 | 59250452 | 59250452 | A > G | Increasing of enhancer |
| | | | | | activity |
| JUN | chr1 | 59251097 | 59251097 | T > G | Increasing of enhancer |
| | | | | | activity |
| KCNQ1 | chr11 | 2847069 | 2847069 | T > G | |
| KCNQ1 | | NA | NA | | Gain of enhancer |
| KCNQ1 | | NA | NA | | Gain of enhancer |
| KCNQ1 | | NA | NA | | Gain of enhancer |
| KCNQ1 | | NA | NA | | Gain of enhancer |
| KCNQ1 | | NA | NA | | Gain of enhancer |
| KCNQ1 | | NA | NA | | Gain of enhancer |
| KCNQ1 | | NA | NA | | Gain of enhancer |
| KCNQ1 | | NA | NA | | Gain of enhancer |
| KCNQ1 | | NA | NA | | Gain of enhancer |
| KCNQ1 | | NA | NA | | Gain of enhancer |
| KCNQ1 | | NA | NA | | Gain of enhancer |
| KCNQ1 | | NA | NA | | Gain of enhancer |
| KCNQ1 | | NA | NA | | Gain of enhancer |
| KCNQ1 | | NA | NA | | Gain of enhancer |
| KCNQ1 | | NA | NA | | Gain of enhancer |
| KCNQ1 | | NA | NA | | Gain of enhancer |
| KCNQ1 | | NA | NA | | Gain of enhancer |
| KDELR2 | chr7 | 6515150 | 6515150 | A > G | Decreasing of enhancer |
| | | | | | activity |
| KDM5A, | chr12 | 349298 | 349298 | C > T | Increasing of enhancer |
| CCDC77, | | | | | activity |
| LOC1002 | | | | | |
| 88778 | | | | | |
| KLF5 | chr13 | 73880413 | 74042621 | Amplification | Gain of enhancer |
| KLF5 | chr13 | 73880413 | 74042621 | Amplification | Gain of enhancer |
| KLF5 | chr13 | 73880413 | 74042621 | Amplification | Gain of enhancer |
| KLF5 | chr13 | 73880413 | 74042621 | Amplification | Gain of enhancer |
| KLF5 | chr13 | 73880680 | 73990596 | Amplification | Gain of enhancer |
| KLF5 | chr13 | 73880680 | 73990596 | Amplification | Gain of enhancer |
| KLK14, SYT3, | chr19 | 51773802 | 51773802 | A > G | Increasing of enhancer |
| LRRC4B, | | | | | activity |
| IGLON5 | | | | | |
| KRT80 | chr12 | 52345023 | 52345023 | G > T | Change of SP4 binding |
| KRT80 | chr12 | 52345376 | 52345376 | G > T | Change of SP4 binding |
| KRT80 | chr12 | 53459012 | 53459012 | G > C | Change of SP4 binding |
| KRT80 | chr12 | 52417086 | 52417086 | C > T | Change of SP4 binding |
| KRT80 | chr12 | 52417086 | 52417086 | C > T | Change of SP4 binding |
| KRTCAP3, | chr2 | 27730940 | 27730940 | T > T | Increasing of enhancer |
| ZNF513, | | | | | activity |
| FTHL3P, | | | | | |
| SNX17, | | | | | |
| PPM1G, | | | | | |
| NRBP1, | | | | | |
| ZNF512, | | | | | |
| CCDC121, | | | | | |
| SLC4A1AP, | | | | | |
| SUPT7L, | | | | | |
| AGBL5, | | | | | |
| SLC30A3, | | | | | |
| GPN1, | | | | | |
| MPV17, | | | | | |
| GCKR, | | | | | |
| IFT172, | | | | | |
| FNDC4, | | | | | |
| GTF3C2, | | | | | |
| EIF2B4, | | | | | |
| RBKS, BRE, | | | | | |
| C2orf16, | | | | | |
| UCN | | | | | |
| LAMA5 | chr20 | 60909815 | 60909815 | G > C | Change of SP3 binding |
| LAMA5 | chr20 | 60909815 | 60909815 | G > C | Change of SP4 binding |
| LAMA5 | chr20 | 60999979 | 60999979 | T > G | Change of SP3 binding |
| LAMA5 | chr20 | 60999979 | 60999979 | T > G | Change of SP4 binding |
| LAMA5 | chr20 | 61686432 | 61686432 | G > A | Change of SP3 binding |
| LAMA5 | chr20 | 61686432 | 61686432 | G > A | Change of SP4 binding |
| LAMA5 | chr20 | 60878221 | 60878221 | T > G | Change of SP3 binding |
| LAMA5 | chr20 | 60878221 | 60878221 | T > G | Change of SP4 binding |
| LBH | chr2 | 30448344 | 30448344 | T > G | Decreasing of enhancer |
| | | | | | activity |
| LBX1 | chr10 | 102979207 | 102979207 | C > T | |
| LIPA | | NA | NA | | Gain of enhancer |
| LIPA | | NA | NA | | Gain of enhancer |
| LIPA | | NA | NA | | Gain of enhancer |
| LIPA | | NA | NA | | Gain of enhancer |
| LIPA | | NA | NA | | Gain of enhancer |
| LIPA | | NA | NA | | Gain of enhancer |
| LIPA | | NA | NA | | Gain of enhancer |
| LIPA | | NA | NA | | Gain of enhancer |
| LIPA | | NA | NA | | Gain of enhancer |
| LMNB1 | chr5 | 125385805 | 126043053 | Deletion | Enhancer hijacking; Loss of |
| | | | | | intrinsic enhancer; Gain of |
| | | | | | new enhancer |
| LMNB1 | chr5 | 125385805 | 126043053 | Deletion | Enhancer hijacking; Loss of |
| | | | | | intrinsic enhancer; Gain of |
| | | | | | new enhancer |
| LMNB1 | chr5 | 125385805 | 126043053 | Deletion | Enhancer hijacking; Loss of |
| | | | | | intrinsic enhancer; Gain of |
| | | | | | new enhancer |
| LMO1 | chr11 | 8289480 | 8289480 | C > T | Gain of MYB binding; Gain of |
| | | | | | enhancer |
| LMO1 | chr11 | 8255408 | 8255408 | T > G | Gain of GATA motif; |
| | | | | | Increasing of enhancer |
| | | | | | activity |
| LPP, BCL6 | chr3 | 187713718 | 187713718 | T > A | Increasing of enhancer |
| | | | | | activity |
| LRIG1 | chr3 | 66798950 | 66798950 | C > G | Increasing of enhancer |
| | | | | | activity |
| LRP1 | chr12 | 57534470 | 57534470 | T > C | Gain of SREBP-1 motif; |
| | | | | | Increasing of enhancer |
| | | | | | activity |
| LTA | chr6 | 31526856 | 31526856 | T > C | Decreasing of enhancer |
| | | | | | activity |
| MAF | chr16 | 79645989 | 79645989 | C > A | Increasing of enhancer |
| | | | | | activity |
| MAP3K1 | chr5 | 55711489 | 55711489 | C > T | Change of ZNF678 binding |
| MAP3K1 | chr5 | 55724929 | 55724929 | C > G | Change ofTFCP2 binding |
| MAP3K1 | chr5 | 55786238 | 55786238 | G > A | Change of RARB binding |
| MAP3K1 | chr5 | 55786238 | 55786238 | G > A | Change of ZNF501 binding |
| MAP3K1 | chr5 | 55786238 | 55786238 | G > A | Change of ZNF713 binding |
| MAP3K1 | chr5 | 55832915 | 55832915 | T > G | Change of PEG3 binding |
| MAP3K1 | chr5 | 55969689 | 55969689 | G > A | Change of SOX15 binding |
| MAP3K1 | chr5 | 55969689 | 55969689 | G > A | Change of SOX18 binding |
| MAP3K1 | chr5 | 55969689 | 55969689 | G > A | Change of SOX21 binding |
| MAP3K1 | chr5 | 55969689 | 55969689 | G > A | Change of SOX2 binding |
| MAP3K1 | chr5 | 55969689 | 55969689 | G > A | Change of SOX7 binding |
| MAP3K1 | chr5 | 55969689 | 55969689 | G > A | Change of SRY binding |
| MAP3K13 | chr3 | 184919694 | 184919694 | G > C | Change of POU1F1 binding |
| MAP3K13 | chr3 | 184919694 | 184919694 | G > C | Change of ZNF501 binding |
| MAP3K13 | chr3 | 185216716 | 185216716 | C > G | Change of TFCP2 binding |
| MAST2 | chr1 | 46476824 | 46476824 | C > - | Increasing of enhancer |
| | | | | | activity |
| MAST2 | chr1 | 46476865 | 46476865 | C > T | Increasing of enhancer |
| | | | | | activity |
| MAST2 | chr1 | 46476944 | 46476944 | C > T | Increasing of enhancer |
| | | | | | activity |
| MAST2 | chr1 | 46477152 | 46477152 | C > T | Increasing of enhancer |
| | | | | | activity |
| MBL2 | chr10 | 54531685 | 54531685 | C > G | Decreasing of enhancer |
| | | | | | activity |
| MBL2 | chr10 | 54532014 | 54532014 | C > G | Decreasing of enhancer |
| | | | | | activity |
| MECOM, | chr3 | 169150632 | 169150632 | T > C | Increasing of enhancer |
| MYNN, | | | | | activity |
| ARPM1 | | | | | |
| MERTK | chr2 | 112753097 | 112753097 | C > A | Gain of IRF1 motif; |
| | | | | | Decreasing of enhancer |
| | | | | | activity |
| MET | chr7 | 115956171 | 115956171 | C > A | Change of F0XJ2 binding |
| MET | chr7 | 115957320 | 115957320 | G > C | Change of ZNF136 binding |
| MET | chr7 | 115957320 | 115957320 | G > C | Change of ZNF670 binding |
| MET | chr7 | 115994876 | 115994876 | C > T | Change of TEAD1 binding |
| MET | chr7 | 116312444 | 116312444 | G > T | Change of BHLHE41 binding |
| MET | chr7 | 116312444 | 116312444 | G > T | Change of HEYL binding |
| MET | chr7 | 116312444 | 116312444 | G > T | Change of TFAP4 binding |
| MET | chr7 | 116312444 | 116312444 | G > T | Change of USF1 binding |
| MET | chr7 | 116312444 | 116312444 | G > T | Change of USF2 binding |
| MET | chr7 | 116312573 | 116312573 | G > C | Change of E2F1 binding |
| MET | chr7 | 116312573 | 116312573 | G > C | Change of NPAS3 binding |
| MET | chr7 | 116312573 | 116312573 | G > C | Change of SP1 binding |
| MET | chr7 | 116312573 | 116312573 | G > C | Change of SP3 binding |
| MET | chr7 | 116312573 | 116312573 | G > C | Change of SP4 binding |
| MET | chr7 | 116312573 | 116312573 | G > C | Change of ZBTB7B binding |
| MET | chr7 | 116314193 | 116314193 | T > G | Change of ZFX binding |
| MET | chr7 | 116911021 | 116911021 | C > G | Change of MEIS2 binding |
| MET | chr7 | 116911021 | 116911021 | C > G | Change of MEIS3 binding |
| MET | chr7 | 116911021 | 116911021 | C > G | Change of TGIF1 binding |
| MET | chr7 | 116911040 | 116911040 | C > A | Change of ZNF282 binding |
| METRNL | chr17 | 81153381 | 81153381 | G > A | Change of ZNF735 binding |
| METRNL | chr17 | 81153656 | 81153656 | C > T | Change of EGR1 binding |
| METRNL | chr17 | 81153656 | 81153656 | C > T | Change of ZNF735 binding |
| METRNL | chr17 | 81153663 | 81153663 | C > T | Change of ZNF735 binding |
| METRNL | chr17 | 81153381 | 81153381 | G > A | Change of ZNF735 binding |
| METRNL | chr17 | 81153656 | 81153656 | C > T | Change of EGR1 binding |
| METRNL | chr17 | 81153656 | 81153656 | C > T | Change of ZNF735 binding |
| METRNL | chr17 | 81153663 | 81153663 | C > T | Change of ZNF735 binding |
| METRNL | chr17 | 81026079 | 81026079 | C > T | Change of EGR1 binding |
| METRNL | chr17 | 81026112 | 81026112 | G > A | Change of EGR1 binding |
| miR-122 | chr18 | 56085917 | 56085917 | C > A | Decreasing of enhancer |
| | | | | | activity |
| MIR137, | chr1 | 98515539 | 98515539 | T > A | Reducing of YYl binding; |
| MIR2682 | | | | | Decreasing of enhancer |
| | | | | | activity |
| MIR137, | chr1 | 98515539 | 98515539 | T > A | Reducing of YYl binding; |
| MIR2682 | | | | | Decreasing of enhancer |
| | | | | | activity |
| miR-200b | chr1 | 1099342 | 1099342 | C > A | Change of BRCA1; CHD2 motif |
| mir30a | chr6 | 72115621 | 72115621 | G > A | Change of AIRE; Sox motif |
| MLH1 | chr3 | 37035012 | 37035012 | C > A | Gain of HSF1; HSF2 binding; |
| | | | | | Decreasing of enhancer |
| | | | | | activity |
| MLLT4 | | NA | NA | | Gain of enhancer |
| MLLT4 | | NA | NA | | Gain of enhancer |
| MLLT4 | | NA | NA | | Gain of enhancer |
| MLLT4 | | NA | NA | | Gain of enhancer |
| MLLT4 | | NA | NA | | Gain of enhancer |
| MLLT4 | | NA | NA | | Gain of enhancer |
| MLLT4 | | NA | NA | | Gain of enhancer |
| MLLT4 | | NA | NA | | Gain of enhancer |
| MLLT4 | | NA | NA | | Gain of enhancer |
| MLLT4 | | NA | NA | | Gain of enhancer |
| MLLT4 | | NA | NA | | Gain of enhancer |
| MLLT4 | | NA | NA | | Gain of enhancer |
| MLLT4 | | NA | NA | | Gain of enhancer |
| MR1 | chr1 | 181020230 | 181020230 | A > G | |
| MRPS30, | chr5 | 44706498 | 44706498 | A > G | Gain of enhancer |
| FGF10 | | | | | |
| MSMB | chr10 | 51549496 | 51549496 | C > T | Decreasing of enhancer |
| | | | | | activity |
| MSNP1AS | chr5 | 25967703 | 25967703 | C > T | Increasing of enhancer |
| | | | | | activity |
| MTAP, | chr9 | 22124477 | 22124477 | A > G | Loss of STAT1 motif; |
| CDKN2B- | | | | | Decreasing of enhancer |
| AS1, | | | | | activity |
| CDKN2A, | | | | | |
| CDKN2B | | | | | |
| MTHFD1L | | NA | NA | | Gain of enhancer |
| MTHFD1L | | NA | NA | | Gain of enhancer |
| MTHFD1L | | NA | NA | | Gain of enhancer |
| MTHFD1L | | NA | NA | | Gain of enhancer |
| MTHFD1L | | NA | NA | | Gain of enhancer |
| MTHFD1L | | NA | NA | | Gain of enhancer |
| MTHFD1L | | NA | NA | | Gain of enhancer |
| MTHFD1L | | NA | NA | | Gain of enhancer |
| MTHFD1L | | NA | NA | | Gain of enhancer |
| MTHFD1L | | NA | NA | | Gain of enhancer |
| MTHFD1L | | NA | NA | | Gain of enhancer |
| MUC1, | chr1 | 150951477 | 150951477 | T > C | Increasing of enhancer |
| C1orf54, | | | | | activity |
| THBS3, | | | | | |
| PSMB4, GBA, | | | | | |
| GBAP, | | | | | |
| APH1A, | | | | | |
| CTSS, | | | | | |
| ANP32E, | | | | | |
| ECM1, | | | | | |
| C1orf51, | | | | | |
| NBPF10, | | | | | |
| CA14, | | | | | |
| LYSMD1, | | | | | |
| CTSK, ARNT, | | | | | |
| SETDB1, | | | | | |
| LASS2, | | | | | |
| ANXA9, | | | | | |
| G0LPH3L, | | | | | |
| PRUNE, | | | | | |
| C1orf56, | | | | | |
| CDC42SE1, | | | | | |
| MLLT11, | | | | | |
| MRPS21, | | | | | |
| PRPF3, | | | | | |
| RPRD2, | | | | | |
| TARS2, | | | | | |
| VPS72, | | | | | |
| ADAMTSL4, | | | | | |
| MCL1, ENSA, | | | | | |
| SEMA6C, | | | | | |
| SCNM1, | | | | | |
| GABPB2, | | | | | |
| PIP5K1A, | | | | | |
| FAM63A, | | | | | |
| PSMD4 | | | | | |
| MYB | NA | NA | NA | Rearrangement | Gain of enhancer |
| MYC | NA | NA | NA | t(3; 8)(q27; q24) | Gain of enhancer |
| MYC | NA | NA | NA | t(3; 8)(q27; q24) | Gain of enhancer |
| MYC | chr8 | 129543949 | 129554294 | Amplification | Gain of enhancer |
| MYC | chr8 | 129166547 | 129190290 | Amplification | Gain of enhancer |
| MYC | chr8 | 128413305 | 128413305 | T > G | Increasing of enhancer |
| | | | | | activity |
| MYC | chr8 | 128407443 | 128407443 | A > G | Increasing of enhancer |
| | | | | | activity |
| MYC | chr8 | 128413305 | 128413305 | T > G | Gain of enhancer activity |
| MYC | chr8 | 128413305 | 128413305 | T > G | Gain of TCF4 motif; Gain of |
| | | | | | enhancer activity |
| MYC | chr8 | 128413305 | 128413305 | T > G | Gain of TCF4 motif; |
| | | | | | Increasing of enhancer |
| | | | | | activity |
| MYC | chr8 | 128413305 | 128413305 | G > T | Gain of TCF7L2 motif; |
| | | | | | Increasing of enhancer |
| | | | | | activity |
| MYC | chr8 | 128413305 | 128413305 | T > G | Gain of TCF7L2 motif; |
| | | | | | Increasing of enhancer |
| | | | | | activity |
| MYC | chr8 | 128413305 | 128413305 | G > T | Gain of TCF7L2 motif; Loss of |
| | | | | | TCF4 motif; Increasing of |
| | | | | | enhancer activity |
| MYC | chr8 | 128413305 | 128413305 | T > G | Increasing of enhancer |
| | | | | | activity |
| MYC | chr8 | 128413305 | 128413305 | | |
| MYC | chr8 | 128413305 | 128413305 | T > G | |
| MYC | chr8 | 128413305 | 128413305 | T > G | Gain of enhancer activity |
| MYC | chr8 | 128654265 | 128654265 | | |
| MYC | chr8 | 128755080 | 128755080 | | Loss of TCF binding; Loss of |
| | | | | | enhancer |
| MYC | chr8 | 130161775 | 130202012 | Duplication | Gain of enhancer |
| MYC | chr8 | 128011937 | 128011937 | T > C | Increasing of enhancer |
| | | | | | activity |
| MYC | chr8 | 128532137 | 128532137 | T > C | Increasing of enhancer |
| | | | | | activity |
| MYC | chr8 | 128532137 | 128532137 | C > T | Increasing of enhancer |
| | | | | | activity |
| MYC | chr8 | 128093297 | 128093297 | C > T | Increasing of enhancer |
| | | | | | activity |
| MYC | chr8 | 128531689 | 128531689 | A > T | Gain of FoxAl motif; |
| | | | | | Increasing of enhancer |
| | | | | | activity |
| MYC | chr8 | 128531873 | 128531873 | G > A | Increasing of enhancer |
| | | | | | activity |
| MYC | chr8 | 127924659 | 127924659 | C > A | Increasing of enhancer |
| | | | | | activity |
| MYC | chr8 | 128095156 | 128095156 | A > G | Increasing of enhancer |
| | | | | | activity |
| MYC | chr8 | 129076573 | 129076573 | C > T | |
| MYC | chr8 | 128485038 | 128485038 | A > C | Increasing of enhancer |
| | | | | | activity |
| MYC | chr8 | 128485038 | 128485038 | C > A | Increasing of enhancer |
| | | | | | activity |
| MYC | chr8 | 128124126 | 128124126 | T > G | Increasing of enhancer |
| | | | | | activity |
| MYC | chr8 | 128124916 | 128124916 | C > A | Increasing of enhancer |
| | | | | | activity |
| MYC | chr8 | 128192981 | 128192981 | A > G | |
| MYC | chr8 | 130645692 | 130645692 | A > G | Gain of enhancer |
| MYC | chr8 | 128335673 | 128335673 | G > A | Increasing of enhancer |
| | | | | | activity |
| MYC | chr8 | 128106880 | 128106880 | A > C | Increasing of enhancer |
| | | | | | activity |
| MYC | chr8 | 129186548 | 129186548 | A > T | |
| MYC | chr8 | 128539360 | 128539360 | T > G | Increasing of enhancer |
| | | | | | activity |
| MYLK | chr3 | 123415781 | 123415781 | G > A | Loss of FOXN1 motif; |
| | | | | | Decreasing of FOXN1 binding; |
| | | | | | Decreasing of enhancer |
| | | | | | activity |
| NBN | chr8 | 91585087 | 91585087 | G > C | Change of HOXC5 binding |
| NBN | chr8 | 91585087 | 91585087 | G > C | Change of PRDM1 binding |
| NBN | chr8 | 91585087 | 91585087 | G > C | Change of ZNF263 binding |
| NCK1 | chr3 | 136801938 | 136801938 | A > C | Increasing of enhancer |
| | | | | | activity |
| NCOR1 | chr17 | 16126784 | 16126784 | A > G | Change of POLR3A binding |
| NCOR1 | chr17 | 16126784 | 16126784 | A > G | Change of TOPORS binding |
| NCOR1 | chr17 | 16128122 | 16128122 | C > T | Change of TP53 binding |
| NCOR1 | chr17 | 16128122 | 16128122 | C > T | Change of TP63 binding |
| NCOR1 | chr17 | 16128122 | 16128122 | C > T | Change of ZNF143 binding |
| NCOR1 | chr17 | 16128122 | 16128122 | C > T | Change of ZNF302 binding |
| NCOR1 | chr17 | 16128122 | 16128122 | C > T | Change of ZNF425 binding |
| NCOR1 | chr17 | 16128122 | 16128122 | C > T | Change of ZNF679 binding |
| NCOR1 | | NA | NA | | Gain of enhancer |
| NCOR1 | | NA | NA | | Gain of enhancer |
| NCOR1 | | NA | NA | | Gain of enhancer |
| NCOR1 | | NA | NA | | Gain of enhancer |
| NCOR1 | | NA | NA | | Gain of enhancer |
| NCOR1 | | NA | NA | | Gain of enhancer |
| NCOR1 | | NA | NA | | Gain of enhancer |
| NCOR1 | | NA | NA | | Gain of enhancer |
| NCOR1 | | NA | NA | | Gain of enhancer |
| NCOR1 | | NA | NA | | Gain of enhancer |
| NCOR1 | | NA | NA | | Gain of enhancer |
| NCOR1 | | NA | NA | | Gain of enhancer |
| NCOR1 | | NA | NA | | Gain of enhancer |
| NEK4 | chr3 | 52819327 | 52819327 | T > A | Decreasing of enhancer |
| | | | | | activity |
| NGEF | chr2 | 233743109 | 233743109 | A > G | Decreasing of enhancer |
| | | | | | activity |
| NKAIN4 | chr20 | 62085791 | 62085791 | G > A | Change of EGR1 binding |
| NKAIN4 | chr20 | 62085791 | 62085791 | G > A | Change of MAZ binding |
| NKAIN4 | chr20 | 62085791 | 62085791 | G > A | Change of SP1 binding |
| NKAIN4 | chr20 | 62085791 | 62085791 | G > A | Change of SP2 binding |
| NKAIN4 | chr20 | 62085791 | 62085791 | G > A | Change of SP3 binding |
| NKAIN4 | chr20 | 62085791 | 62085791 | G > A | Change of ZNF263 binding |
| NKAIN4 | chr20 | 62133292 | 62133292 | T > G | Change of EGR1 binding |
| NKAIN4 | chr20 | 62133292 | 62133292 | T > G | Change of EGR4 binding |
| NKAIN4 | chr20 | 62133292 | 62133292 | T > G | Change of MAZ binding |
| NKAIN4 | chr20 | 62133292 | 62133292 | T > G | Change of SP1 binding |
| NKAIN4 | chr20 | 62133292 | 62133292 | T > G | Change of SP2 binding |
| NKAIN4 | chr20 | 62133292 | 62133292 | T > G | Change of SP3 binding |
| NKAIN4 | chr20 | 62133292 | 62133292 | T > G | Change of SP4 binding |
| NKAIN4 | chr20 | 62133292 | 62133292 | T > G | Change of ZNF263 binding |
| NKAIN4 | chr20 | 62133292 | 62133292 | T > G | Change of ZNF281 binding |
| NKAIN4 | chr20 | 61962472 | 61962472 | A > G | Change of SP1 binding |
| NKAIN4 | chr20 | 61962472 | 61962472 | A > G | Change of SP3 binding |
| NKAIN4 | chr20 | 61962472 | 61962472 | A > G | Change of SP4 binding |
| NKAIN4 | chr20 | 61885983 | 61885983 | A > C | Change of EGR1 binding |
| NKAIN4 | chr20 | 61885983 | 61885983 | A > C | Change of EGR4 binding |
| NKAIN4 | chr20 | 61885983 | 61885983 | A > C | Change of MAZ binding |
| NKAIN4 | chr20 | 61885983 | 61885983 | A > C | Change of SP1 binding |
| NKAIN4 | chr20 | 61885983 | 61885983 | A > C | Change of SP2 binding |
| NKAIN4 | chr20 | 61885983 | 61885983 | A > C | Change of SP3 binding |
| NKAIN4 | chr20 | 61885983 | 61885983 | A > C | Change of SP4 binding |
| NKAIN4 | chr20 | 61885983 | 61885983 | A > C | Change of ZNF263 binding |
| NKAIN4 | chr20 | 61885983 | 61885983 | A > C | Change of ZNF281 binding |
| NKAIN4 | chr20 | 61806945 | 61806945 | T > G | Change of EGR1 binding |
| NKAIN4 | chr20 | 61806945 | 61806945 | T > G | Change of SP1 binding |
| NKAIN4 | chr20 | 61806945 | 61806945 | T > G | Change of ZNF281 binding |
| NKAIN4 | chr20 | 61898173 | 61898173 | C > A | Change of EGR4 binding |
| NOG | chr17 | 54773238 | 54773238 | A > T | Loss ofMEFZCand CDX2 |
| | | | | | binding ; Loss of enhancer |
| | | | | | activity |
| NOS1AP | chr1 | 162021000 | 162021000 | A > G | Increasing of enhancer |
| | | | | | activity |
| NOS1AP | chr1 | 162020969 | 162020969 | A > T | Increasing of enhancer |
| | | | | | activity |
| NOS2 | chr17 | 26132301 | 26139107 | Duplication | Gain of enhancer |
| NOS2 | chr7 | 26132301 | 26139107 | Duplication | Gain of enhancer |
| NOTCH1 | chr9 | 139414453 | 139414453 | T > G | Change of EGR4 binding |
| NOTCH1 | chr9 | 139414453 | 139414453 | T > G | Change of GLI2 binding |
| NOTCH1 | chr9 | 139414453 | 139414453 | T > G | Change of KLF1 binding |
| NOTCH1 | chr9 | 139414453 | 139414453 | T > G | Change of WT1 binding |
| NOTCH1 | chr9 | 139414453 | 139414453 | T > G | Change ofZBTB11 binding |
| NOTCH1 | chr9 | 139414453 | 139414453 | T > G | Change of ZBTB7B binding |
| NOTCH1 | chr9 | 139414453 | 139414453 | T > G | Change of ZNF837 binding |
| NOTCH1 | chr9 | 139460020 | 139460020 | G > T | Change of ZEB1 binding |
| NOTCH1 | chr9 | 139460020 | 139460020 | G > T | Change of ZNF555 binding |
| NOTCH1 | chr9 | 139460279 | 139460279 | G > C | Change of ZAC binding |
| NOTCH1 | chr9 | 139460279 | 139460279 | G > C | Change of ZNF208 binding |
| NOTCH1 | chr9 | 139492936 | 139492936 | G > C | Change of KLF15 binding |
| NOTCH1 | chr9 | 139492936 | 139492936 | G > C | Change of KLF16 binding |
| NOTCH1 | chr9 | 139492936 | 139492936 | G > C | Change of ZNF263 binding |
| NOTCH1 | chr9 | 139492936 | 139492936 | G > C | Change of ZNF599 binding |
| NOTCH1 | chr9 | 139503959 | 139503959 | C > G | Change of ZNF283 binding |
| NOTCH1 | chr9 | 139503959 | 139503959 | C > G | Change of ZNF729 binding |
| NOTCH1 | chr9 | 139503959 | 139503959 | C > G | Change of ZNF814 binding |
| NOTCH1 | chr9 | 139512789 | 139512789 | A > G | Change of TFCP2 binding |
| NOTCH1 | chr9 | 139512789 | 139512789 | A > G | Change of TP73 binding |
| NOTCH1 | chr9 | 139512789 | 139512789 | A > G | Change of ZNF80 binding |
| NOTCH1 | chr9 | 139513656 | 139513656 | G > A | Change of FOXRI binding |
| NOTCH1 | chr9 | 139929812 | 139929812 | G > A | Change of NHLH2 binding |
| NOTCH1 | chr9 | 139929812 | 139929812 | G > A | Change of TCF12 binding |
| NOTCH1 | chr9 | 139929812 | 139929812 | G > A | Change ofTCF3 binding |
| NOTCH1 | chr9 | 139929812 | 139929812 | G > A | Change of ZNF524 binding |
| NOTCH2 | chr1 | 121112052 | 121112052 | G > C | |
| NR3C1 | chr5 | 142782299 | 142782299 | C > T | Change of SP4 binding |
| NR3C1 | chr5 | 142782658 | 142782658 | A > C | Change of SP1 binding |
| NR3C1 | chr5 | 142782658 | 142782658 | A > C | Change of SP3 binding |
| NR3C1 | chr5 | 142782658 | 142782658 | A > C | Change of SP4 binding |
| NR3C1 | chr5 | 142782995 | 142782995 | G > A | Change of SP1 binding |
| NR3C1 | chr5 | 142782995 | 142782995 | G > A | Change of SP3 binding |
| NR3C1 | chr5 | 142782995 | 142782995 | G > A | Change of SP4 binding |
| NR3C1 | chr5 | 142783252 | 142783252 | G > T | Change of SP1 binding |
| NR3C1 | chr5 | 142783252 | 142783252 | G > T | Change of SP3 binding |
| NR3C1 | chr5 | 142783252 | 142783252 | G > T | Change of SP4 binding |
| NR3C1 | chr5 | 142783284 | 142783284 | T > C | Change of SP1 binding |
| NR3C1 | chr5 | 142783284 | 142783284 | T > C | Change of SP3 binding |
| NR3C1 | chr5 | 142783284 | 142783284 | T > C | Change of SP4 binding |
| NR3C1 | chr5 | 142783293 | 142783293 | A > C | Change of SP1 binding |
| NR3C1 | chr5 | 142783293 | 142783293 | A > C | Change of SP3 binding |
| NR3C1 | chr5 | 142783293 | 142783293 | A > C | Change of SP4 binding |
| NR3C1 | chr5 | 142783319 | 142783319 | G > A | Change of SP4 binding |
| NR3C1 | chr5 | 142783408 | 142783408 | G > C | Change of SP1 binding |
| NR3C1 | chr5 | 142783408 | 142783408 | G > C | Change of SP3 binding |
| NR3C1 | chr5 | 142783408 | 142783408 | G > C | Change of SP4 binding |
| NR3C1 | chr5 | 142783464 | 142783464 | T > G | Change of SP3 binding |
| NR3C1 | chr5 | 142783464 | 142783464 | T > G | Change of SP4 binding |
| NR3C1 | chr5 | 142783466 | 142783466 | G > A | Change of SP1 binding |
| NR3C1 | chr5 | 142783466 | 142783466 | G > A | Change of SP3 binding |
| NR3C1 | chr5 | 142783466 | 142783466 | G > A | Change of SP4 binding |
| NR3C1 | chr5 | 142783474 | 142783474 | G > C | Change of SP1 binding |
| NR3C1 | chr5 | 142783474 | 142783474 | G > C | Change of SP3 binding |
| NR3C1 | chr5 | 142783474 | 142783474 | G > C | Change of SP4 binding |
| NR3C1 | chr5 | 141228722 | 141228722 | C > G | Change of SP1 binding |
| NR3C1 | chr5 | 141228722 | 141228722 | C > G | Change of SP3 binding |
| NR3C1 | chr5 | 141228722 | 141228722 | C > G | Change of SP4 binding |
| NR3C1 | chr5 | 141229405 | 141229405 | G > A | Change of SP1 binding |
| NR3C1 | chr5 | 141229405 | 141229405 | G > A | Change of SP3 binding |
| NR3C1 | chr5 | 141229405 | 141229405 | G > A | Change of SP4 binding |
| NRAS | chr1 | 115300152 | 115300152 | T > G | Change of ELF3 binding |
| NRAS | chr1 | 115300152 | 115300152 | T > G | Change of FOXO6 binding |
| NRAS | chr1 | 115300152 | 115300152 | T > G | Change of STAT2 binding |
| NRAS | chr1 | 115300152 | 115300152 | T > G | Change of ZNF708 binding |
| NRAS | chr1 | 115300224 | 115300224 | C > T | Change of EGR4 binding |
| NRAS | chr1 | 115300224 | 115300224 | C > T | Change of PRDM9 binding |
| NRAS | chr1 | 115300224 | 115300224 | C > T | Change of SP1 binding |
| NRAS | chr1 | 115300224 | 115300224 | C > T | Change of SP3 binding |
| NRAS | chr1 | 115300224 | 115300224 | C > T | Change of SP4 binding |
| NRAS | chr1 | 115300224 | 115300224 | C > T | Change of WT1 binding |
| NRAS | chr1 | 115300224 | 115300224 | C > T | Change of ZNF148 binding |
| NRAS | chr1 | 115300224 | 115300224 | C > T | Change of ZNF790 binding |
| NRG1 | chr8 | 31495581 | 31495581 | C > T | Gain of ZNF581; ZNF524; |
| | | | | | FOXH1 binding; Decreasing of |
| | | | | | enhancer activity |
| NRGN | chr11 | 124609907 | 124609907 | C > G | Gain of NR2C2 binding; |
| | | | | | Decreasing of enhancer |
| | | | | | activity |
| NSD1, | chr5 | 176817636 | 176817636 | A > G | Increasing of enhancer |
| MXD3, | | | | | activity |
| LMAN2, | | | | | |
| RAB24, | | | | | |
| PRELID1, | | | | | |
| GRK6, PRR7, | | | | | |
| RGS14, F12, | | | | | |
| DOK3, | | | | | |
| DDX41, | | | | | |
| FAM193B, | | | | | |
| UIMC1, | | | | | |
| FGFR4, | | | | | |
| SLC34A1, | | | | | |
| TMED9, | | | | | |
| B4GALT7, | | | | | |
| PDLIM7, | | | | | |
| DBN1 | | | | | |
| NT5C2 | chr10 | 104653545 | 104653545 | T > C | Decreasing of enhancer |
| | | | | | activity |
| NUB1, RHEB, | chr7 | 151407801 | 151407801 | G > A | Increasing of enhancer |
| PRKAG2 | | | | | activity |
| NUDT11 | chrX | 51505931 | 51505931 | T > C | Increasing of enhancer |
| | | | | | activity |
| NXT1, CST1, | chr20 | 23612737 | 23612737 | C > C | Increasing of enhancer |
| GZF1, NAPB, | | | | | activity |
| CST3, CST2, | | | | | |
| CST5, | | | | | |
| ENTPD6, | | | | | |
| PYGB, CST8, | | | | | |
| CST4, SSTR4, | | | | | |
| THBD | | | | | |
| OCIAD2 | chr4 | 49126582 | 49126582 | T > A | Change of ZNF616 binding |
| OCIAD2 | chr4 | 49126899 | 49126899 | C > G | Change of ZNF616 binding |
| OCIAD2 | chr4 | 49127870 | 49127870 | A > T | Change of ZNF616 binding |
| OCIAD2 | chr4 | 49141001 | 49141001 | C > A | Change of ZNF616 binding |
| OCIAD2 | chr4 | 49142162 | 49142162 | A > C | Change of ZNF616 binding |
| OCIAD2 | chr4 | 49142162 | 49142162 | A > C | Change of ZNF616 binding |
| ONECUT1 | chr15 | 53942928 | 53942928 | T > C | Increasing of enhancer |
| | | | | | activity |
| ORMDL3 | chr17 | 38029120 | 38029120 | G > C | Loss of CTCF motif; |
| | | | | | Decreasing of CTCF binding; |
| | | | | | Increasing of enhancer |
| | | | | | activity |
| ORMDL3 | chr17 | 38080865 | 38080865 | A > G | Gain of CTCF motif; |
| | | | | | Increasing of CTCF binding; |
| | | | | | Increasing of enhancer |
| | | | | | activity |
| OSGIN1 | | NA | NA | | Gain of enhancer |
| OSGIN1 | | NA | NA | | Gain of enhancer |
| OSGIN1 | | NA | NA | | Gain of enhancer |
| OSGIN1 | | NA | NA | | Gain of enhancer |
| OSGIN1 | | NA | NA | | Gain of enhancer |
| OSGIN1 | | NA | NA | | Gain of enhancer |
| OSGIN1 | | NA | NA | | Gain of enhancer |
| OSGIN1 | | NA | NA | | Gain of enhancer |
| OSGIN1 | | NA | NA | | Gain of enhancer |
| OSGIN1 | | NA | NA | | Gain of enhancer |
| OSGIN1 | | NA | NA | | Gain of enhancer |
| OSGIN1 | | NA | NA | | Gain of enhancer |
| OSGIN1 | | NA | NA | | Gain of enhancer |
| PANK4 | chr1 | 2615274 | 2615274 | C > T | Change of RREB1 binding |
| PANK4 | chr1 | 2615274 | 2615274 | C > T | Change of SP3 binding |
| PANK4 | chr1 | 2617111 | 2617111 | C > A | Change of RREB1 binding |
| PANK4 | chr1 | 2617111 | 2617111 | C > A | Change of SP3 binding |
| PANK4 | chr1 | 2617159 | 2617159 | C > A | Change of RREB1 binding |
| PANK4 | chr1 | 2617159 | 2617159 | C > A | Change of SP3 binding |
| PANK4 | chr1 | 2617877 | 2617877 | C > A | Change of RREB1 binding |
| PANK4 | chr1 | 2617877 | 2617877 | C > A | Change of SP3 binding |
| PANK4 | chr1 | 2618557 | 2618557 | C > G | Change of RREB1 binding |
| PANK4 | chr1 | 2618557 | 2618557 | C > G | Change of SP3 binding |
| PANK4 | chr1 | 2246358 | 2246358 | T > G | Change of RREB1 binding |
| PANK4 | chr1 | 2246358 | 2246358 | T > G | Change of SP3 binding |
| PARD6B | chr20 | 48997377 | 49019434 | Amplification | Gain of enhancer |
| PARK2 | chr6 | 163212578 | 163212578 | G > A | Gain of FOXA motif; |
| | | | | | Decreasing of enhancer |
| | | | | | activity |
| PARK2 | chr6 | 163212465 | 163212465 | T > C | Gain of XBP-1 motif; Loss of |
| | | | | | SOX motif; Decreasing of |
| | | | | | enhancer activity |
| PAX6 | NA | NA | NA | Deletion | Loss of enhancer |
| PAX6 | chr11 | 31642266 | 31685847 | Deletion | Loss of enhancer |
| PBRM1 | chr3 | 52211851 | 52211851 | G > A | Change of ESR1 binding |
| PBRM1 | chr3 | 52211851 | 52211851 | G > A | Change of ESR2 binding |
| PBRM1 | chr3 | 52211851 | 52211851 | G > A | Change of ZEB1 binding |
| PBRM1 | chr3 | 52274776 | 52274776 | C > G | Change ofTCF3 binding |
| PBRM1 | chr3 | 52274776 | 52274776 | C > G | Change of ZNF211 binding |
| PBRM1 | chr3 | 52726385 | 52726385 | A > T | Change of FOXP4 binding |
| PBRM1 | chr3 | 52732863 | 52732863 | G > C | Change of FU20557 binding |
| PBRM1 | chr3 | 52732863 | 52732863 | G > C | Change of ZNF836 binding |
| PCAT1 | chr8 | 128104055 | 128104055 | G > T | Gain of ONECUT2 motif; |
| | | | | | Increasing of enhancer |
| | | | | | activity |
| PDE4B | chr1 | 66746784 | 66746784 | G > A | Increasing of enhancer |
| | | | | | activity |
| PEX10 | chr1 | 2475021 | 2475021 | C > T | Change of SP4 binding |
| PEX10 | chr1 | 2475021 | 2475021 | C > T | Change of SP4 binding |
| PEX10 | chr1 | 2348267 | 2348267 | G > A | Change of SP4 binding |
| PEX10 | chr1 | 2246358 | 2246358 | T > G | Change of SP4 binding |
| PEX10 | chr1 | 2246916 | 2246916 | A > C | Change of SP4 binding |
| PEX6 | chr6 | 42928461 | 42928461 | C > T | Loss of enhancer |
| PFKFB3 | chr10 | 6101713 | 6101713 | C > T | Increasing of enhancer |
| | | | | | activity |
| PFKFB3 | chr10 | 6100725 | 6100725 | A > G | Increasing of enhancer |
| | | | | | activity |
| PFKFB3 | chr10 | 6098949 | 6098949 | C > T | Decreasing of enhancer |
| | | | | | activity |
| PFKFB3 | chr10 | 6101713 | 6101713 | C > T | Increasing of enhancer |
| | | | | | activity |
| PFKFB3 | chr10 | 6100725 | 6100725 | A > G | Increasing of enhancer |
| | | | | | activity |
| PFKFB3 | chr10 | 6098949 | 6098949 | C > T | Decreasing of enhancer |
| | | | | | activity |
| PFKFB3 | chr10 | 6101713 | 6101713 | C > T | Increasing of enhancer |
| | | | | | activity |
| PFKFB3 | chr10 | 6100725 | 6100725 | A > G | Increasing of enhancer |
| | | | | | activity |
| PFKFB3 | chr10 | 6098949 | 6098949 | C > T | Decreasing of enhancer |
| | | | | | activity |
| PFKFB3 | chr10 | 6101713 | 6101713 | C > T | Increasing of enhancer |
| | | | | | activity |
| PFKFB3 | chr10 | 6100725 | 6100725 | A > G | Increasing of enhancer |
| | | | | | activity |
| PFKFB3 | chr10 | 6098949 | 6098949 | C > T | Decreasing of enhancer |
| | | | | | activity |
| PFKFB3 | chr10 | 6101713 | 6101713 | C > T | Increasing of enhancer |
| | | | | | activity |
| PFKFB3 | chr10 | 6100725 | 6100725 | A > G | Increasing of enhancer |
| | | | | | activity |
| PFKFB3 | chr10 | 6098949 | 6098949 | C > T | Decreasing of enhancer |
| | | | | | activity |
| PFKFB3 | chr10 | 6101713 | 6101713 | C > T | Increasing of enhancer |
| | | | | | activity |
| PFKFB3 | chr10 | 6100725 | 6100725 | A > G | Increasing of enhancer |
| | | | | | activity |
| PFKFB3 | chr10 | 6098949 | 6098949 | C > T | Decreasing of enhancer |
| | | | | | activity |
| PFKFB3 | chr10 | 6110875 | 6110875 | A > C | Increasing of enhancer |
| | | | | | activity |
| PFKFB3 | chr10 | 6110875 | 6110875 | A > C | Increasing of enhancer |
| | | | | | activity |
| PFKFB3 | chr10 | 6110875 | 6110875 | A > C | Increasing of enhancer |
| | | | | | activity |
| PFKFB3 | chr10 | 6110875 | 6110875 | A > C | Increasing of enhancer |
| | | | | | activity |
| PFKFB3 | chr10 | 6110875 | 6110875 | A > C | Increasing of enhancer |
| | | | | | activity |
| PFKFB3 | chr10 | 6110875 | 6110875 | A > C | Increasing of enhancer |
| | | | | | activity |
| Pgl3 | chr11 | 2186335 | 2186335 | T > C | Gain of enhancer |
| PHACTR1 | chr6 | 12903957 | 12903957 | A > G | Loss of MEF2A motif; |
| | | | | | Decreasing of enhancer |
| | | | | | activity |
| PHKG2, | chr16 | 30669784 | 30669784 | G > C | Change of SP3 binding |
| C16orf93 | | | | | |
| PHKG2, | chr16 | 30753142 | 30753142 | C > T | Change of SP3 binding |
| C16orf93 | | | | | |
| PHKG2, | chr16 | 30935409 | 30935409 | C > G | Change of SP3 binding |
| C16orf93 | | | | | |
| PHKG2, | chr16 | 31153547 | 31153547 | A > C | Change of SP3 binding |
| C16orf93 | | | | | |
| PIGM | chr1 | 160001799 | 160001799 | G > C | Gain of MIZF binding; |
| | | | | | Decreasing of enhancer |
| | | | | | activity |
| PIK3CA | chr3 | 179169002 | 179169002 | C > A | Change of EGR3 binding |
| PIK3CA | chr3 | 179169002 | 179169002 | C > A | Change of EGR4 binding |
| PIK3CA | chr3 | 179169002 | 179169002 | C > A | Change of FKLF binding |
| PIK3CA | chr3 | 179169002 | 179169002 | C > A | Change of FOXN4 binding |
| PIK3CA | chr3 | 179169002 | 179169002 | C > A | Change of KLF4 binding |
| PIK3CA | chr3 | 179169002 | 179169002 | C > A | Change of KLF5 binding |
| PIK3CA | chr3 | 179169002 | 179169002 | C > A | Change of KLF7 binding |
| PIK3CA | chr3 | 179169002 | 179169002 | C > A | Change of KLF8 binding |
| PIK3CA | chr3 | 179169002 | 179169002 | C > A | Change of OSX binding |
| PIK3CA | chr3 | 179169002 | 179169002 | C > A | Change of SP1 binding |
| PIK3CA | chr3 | 179169002 | 179169002 | C > A | Change of SP2 binding |
| PIK3CA | chr3 | 179169002 | 179169002 | C > A | Change of SP3 binding |
| PIK3CA | chr3 | 179169002 | 179169002 | C > A | Change of SP4 binding |
| PIK3CA | chr3 | 179169002 | 179169002 | C > A | Change of SP5 binding |
| PIK3CA | chr3 | 179169002 | 179169002 | C > A | Change of SP8 binding |
| PIK3CA | chr3 | 179169002 | 179169002 | C > A | Change of SP9 binding |
| PIK3CA | chr3 | 179169002 | 179169002 | C > A | Change of TIEG1 binding |
| PIK3CA | chr3 | 179169002 | 179169002 | C > A | Change of ZBTB7B binding |
| PIK3CA | chr3 | 179169002 | 179169002 | C > A | Change of ZNF143 binding |
| PIK3CA | chr3 | 179169002 | 179169002 | C > A | Change of ZNF460 binding |
| PIK3CA | chr3 | 179169300 | 179169300 | C > G | Change of EGR1 binding |
| PIK3CA | chr3 | 179169300 | 179169300 | C > G | Change of SP1 binding |
| PIK3CA | chr3 | 179169300 | 179169300 | C > G | Change of SP3 binding |
| PIK3CA | chr3 | 179169300 | 179169300 | C > G | Change of SP4 binding |
| PIK3CA | chr3 | 179169300 | 179169300 | C > G | Change of ZBTB7B binding |
| PIK3CA | chr3 | 179169300 | 179169300 | C > G | Change of ZNF148 binding |
| PIK3CA | chr3 | 179169300 | 179169300 | C > G | Change of ZNF325 binding |
| PIK3CA | chr3 | 179169404 | 179169404 | A > G | Change of E2F1 binding |
| PIK3CA | chr3 | 179169404 | 179169404 | A > G | Change of EGR4 binding |
| PIK3CA | chr3 | 179169404 | 179169404 | A > G | Change of SP4 binding |
| PIK3CA | chr3 | 179169404 | 179169404 | A > G | Change of WT1 binding |
| PIK3CA | chr3 | 179169404 | 179169404 | A > G | Change of ZBTB7B binding |
| PIK3CA | chr3 | 179169418 | 179169418 | G > A | Change of EGR4 binding |
| PIK3CA | chr3 | 179169418 | 179169418 | G > A | Change of MAZ binding |
| PIK3CA | chr3 | 179169418 | 179169418 | G > A | Change of PLAG1 binding |
| PIK3CA | chr3 | 179169418 | 179169418 | G > A | Change of SP1 binding |
| PIK3CA | chr3 | 179169418 | 179169418 | G > A | Change of SP3 binding |
| PIK3CA | chr3 | 179169418 | 179169418 | G > A | Change of SP4 binding |
| PIK3CA | chr3 | 179169418 | 179169418 | G > A | Change of ZBTB7A binding |
| PIK3CA | chr3 | 179169418 | 179169418 | G > A | Change of ZBTB7B binding |
| PIK3CA | chr3 | 179169418 | 179169418 | G > A | Change of ZNF350 binding |
| PIK3CA | chr3 | 179169418 | 179169418 | G > A | Change of ZNF660 binding |
| PIK3CA | chr3 | 179169418 | 179169418 | G > A | Change of ZNF841 binding |
| PIN1 | chr19 | 9945179 | 9945179 | G > C | Gain of ZDHHC7 binding; |
| | | | | | Decreasing of enhancer |
| | | | | | activity |
| PITX1 | NA | NA | NA | Duplication | Gain of enhancer |
| PITX2 | chr4 | 111710169 | 111710169 | C > T | Decreasing of enhancer |
| | | | | | activity |
| PKLR | chr1 | 155271258 | 155271258 | T > C | Decreasing of enhancer |
| | | | | | activity |
| PLEK | chr2 | 68647095 | 68647095 | C > T | |
| PLG | chr6 | 161143608 | 161143608 | C > T | Increasing of enhancer |
| | | | | | activity |
| POLH, | chr6 | 43806609 | 43806609 | G > G | Increasing of enhancer |
| MRPS18A, | | | | | activity |
| VEGFA, | | | | | |
| MAD2L1BP, | | | | | |
| RSPH9, | | | | | |
| GTPBP2, | | | | | |
| CAPN11, | | | | | |
| ZNF318, | | | | | |
| MRPL14, | | | | | |
| TMEM63B, | | | | | |
| XPO5, | | | | | |
| HSP90AB1, | | | | | |
| NFKBIE, | | | | | |
| TMEM151B, | | | | | |
| CDC5L, | | | | | |
| SLC29A1, | | | | | |
| DLK2, TJAP1 | | | | | |
| POU3F4 | chrX | 81727184 | 81731085 | Deletion | Loss of enhancer |
| POU3F4 | chrX | 81840528 | 81844429 | Deletion | Loss of enhancer |
| POU5F1B | chr8 | 128407443 | 128407443 | A > G | Decreasing of enhancer |
| | | | | | activity |
| POU5F1B | chr8 | 128413305 | 128413305 | G > T | Decreasing of enhancer |
| | | | | | activity |
| POU5F1B | chr8 | 128424792 | 128424792 | A > G | Decreasing of enhancer |
| | | | | | activity |
| PPARG | chr3 | 12386337 | 12386337 | | |
| PPM1N | chr19 | 45981772 | 45981772 | C > G | Change of CTCF binding |
| PPM1N | chr19 | 45981775 | 45981775 | G > T | Change of CTCF binding |
| PPM1N | chr19 | 45981772 | 45981772 | C > G | Change of CTCF binding |
| PPM1N | chr19 | 45981775 | 45981775 | G > T | Change of CTCF binding |
| PPM1N | chr19 | 45972294 | 45972294 | C > T | Change of CTCF binding |
| PPM1N | chr19 | 46319995 | 46319995 | G > T | Change of CTCF binding |
| PPM1N | chr19 | 45954137 | 45954137 | G > C | Change of SP4 binding |
| PPM1N | chr19 | 45954137 | 45954137 | G > C | Change of SP4 binding |
| PPM1N | chr19 | 45394224 | 45394224 | G > T | Change of SP4 binding |
| PPM1N | chr19 | 45394538 | 45394538 | G > T | Change of SP4 binding |
| PPM1N | chr19 | 45394627 | 45394627 | G > C | Change of SP4 binding |
| PPM1N | chr19 | 45942991 | 45942991 | C > T | Change of SP4 binding |
| PPM1N | chr19 | 45349271 | 45349271 | G > A | Change of SP4 binding |
| PPM1N | chr19 | 45349297 | 45349297 | C > A | Change of SP4 binding |
| PPM1N | chr19 | 45811530 | 45811530 | T > G | Change of SP4 binding |
| PPP3CA | | NA | NA | | Gain of enhancer |
| PPP3CA | | NA | NA | | Gain of enhancer |
| PPP3CA | | NA | NA | | Gain of enhancer |
| PPP3CA | | NA | NA | | Gain of enhancer |
| PPP3CA | | NA | NA | | Gain of enhancer |
| PPP3CA | | NA | NA | | Gain of enhancer |
| PPP3CA | | NA | NA | | Gain of enhancer |
| PPP3CA | | NA | NA | | Gain of enhancer |
| PPP3CA | | NA | NA | | Gain of enhancer |
| PPP3CA | | NA | NA | | Gain of enhancer |
| PPP3CA | | NA | NA | | Gain of enhancer |
| PPP3CA | | NA | NA | | Gain of enhancer |
| PPP3CA | | NA | NA | | Gain of enhancer |
| PPP3CA | | NA | NA | | Gain of enhancer |
| PPP4R1L, | chr20 | 57469073 | 57469073 | T > T | Increasing of enhancer |
| ATP5E, | | | | | activity |
| NPEPL1, | | | | | |
| STX16, | | | | | |
| RAB22A, | | | | | |
| GNAS, VAPB, | | | | | |
| AURKA, | | | | | |
| SLMO2, | | | | | |
| TH1L, CTSZ | | | | | |
| PREX1 | chr20 | 47375521 | 47375521 | C > T | Change of EGR4 binding |
| PREX1 | chr20 | 47375521 | 47375521 | C > T | Change of EGR4 binding |
| PREX1 | chr20 | 47120374 | 47120374 | C > A | Change of EGR4 binding |
| PRKCA | chr17 | 64306133 | 64306133 | T > C | Decreasing of TF binding; |
| | | | | | Decreasing of enhancer |
| | | | | | activity |
| PROP1 | chr5 | 177421909 | 177421909 | delATG | Decreasing of enhancer |
| | | | | | activity |
| PROP1 | chr5 | 177422391 | 177422391 | G > A | Decreasing of enhancer |
| | | | | | activity |
| PRPF40A | chr2 | 113978940 | 113978940 | C > G | Increasing of enhancer |
| | | | | | activity |
| PRRX1 | chr1 | 170587340 | 170587340 | C > T | Increasing of enhancer |
| | | | | | activity |
| PSEN1 | chr14 | 73602899 | 73602899 | C > G | Gain of TBX20 binding; |
| | | | | | Decreasing of enhancer |
| | | | | | activity |
| PTCHD1 | | NA | NA | | Gain of enhancer |
| PTCHD1 | | NA | NA | | Gain of enhancer |
| PTCHD1 | | NA | NA | | Gain of enhancer |
| PTCHD1 | | NA | NA | | Gain of enhancer |
| PTCHD1 | | NA | NA | | Gain of enhancer |
| PTCHD1 | | NA | NA | | Gain of enhancer |
| PTCHD1 | | NA | NA | | Gain of enhancer |
| PTCHD1 | | NA | NA | | Gain of enhancer |
| PTCHD1 | | NA | NA | | Gain of enhancer |
| PTCHD1 | | NA | NA | | Gain of enhancer |
| PTCHD1 | | NA | NA | | Gain of enhancer |
| PTEN | chr10 | 89622988 | 89622988 | A > G | Decreasing of enhancer |
| | | | | | activity |
| PTEN | chr10 | 89623392 | 89623392 | C > T | Decreasing of enhancer |
| | | | | | activity |
| PTEN | chr10 | 89623462 | 89623462 | A > G | Decreasing of enhancer |
| | | | | | activity |
| PTEN | chr10 | 89872831 | 89872831 | G > A | Change of PPARG binding |
| PTEN | chr10 | 89913137 | 89913137 | T > C | Change of GATA5 binding |
| PTEN | chr10 | 89913137 | 89913137 | T > C | Change of HNF1B binding |
| PTEN | chr10 | 89938170 | 89938170 | C > A | Change of ZNF274 binding |
| PTEN | chr10 | 90146219 | 90146219 | C > T | Change of ZSCAN5C binding |
| PTEN | chr17 | 5519006 | 5519006 | A > G | |
| PTF1A | chr10 | 23508305 | 23508305 | A > G | Loss of FOXA2 motif; Loss of |
| | | | | | enhancer |
| PTF1A | chr10 | 23508363 | 23508363 | A > G | Loss of FOXA2 motif; Loss of |
| | | | | | enhancer |
| PTF1A | chr10 | 23508363 | 23508363 | A > G | Loss of FOXA2 motif; Loss of |
| | | | | | enhancer |
| PTF1A | chr10 | 23508365 | 23508365 | A > G | Loss of enhancer |
| PTF1A | chr10 | 23508437 | 23508437 | A > G | Loss of FOXA2 motif; Loss of |
| | | | | | enhancer |
| PTF1A | chr10 | 23508446 | 23508446 | A > C | Loss of PDX1 motif; Loss of |
| | | | | | enhancer |
| PTF1A | chr10 | 23508446 | 23508446 | A > C | Loss of PDX1 motif; Loss of |
| | | | | | enhancer |
| PTK2B, CLU, | chr8 | 27456253 | 27456253 | A > G | Loss of HSF1 binding; |
| EPHX2 | | | | | Increasing of enhancer |
| | | | | | activity |
| PVT1 | chr8 | 129106383 | 129106383 | | |
| PVT1 | chr8 | 128323819 | 128323819 | A > G | Decreasing of YYl binding; |
| | | | | | Increasing of enhancer |
| | | | | | activity |
| RAB22A | chr20 | 56942557 | 56942557 | A > G | Increasing of enhancer |
| | | | | | activity |
| RAD21 | chr8 | 117778780 | 117778780 | G > A | Change of HES4 binding |
| RAD21 | chr8 | 117778780 | 117778780 | G > A | Change of NPAS3 binding |
| RAD21 | chr8 | 117778780 | 117778780 | G > A | Change of TFCP2 binding |
| RAD21 | chr8 | 117778780 | 117778780 | G > A | Change of ZNF677 binding |
| RAD21 | chr8 | 118742743 | 118742743 | G > A | Change of LMO2 binding |
| RAD21 | chr8 | 118742743 | 118742743 | G > A | Change of NHLH2 binding |
| RAD21 | chr8 | 118742743 | 118742743 | G > A | Change of SZF1 binding |
| RAD21 | chr8 | 118742743 | 118742743 | G > A | Change of ZEB1 binding |
| RAD21 | chr8 | 118742743 | 118742743 | G > A | Change of ZNF367 binding |
| RAD21 | chr8 | 118743315 | 118743315 | A > G | Change of FOXP4 binding |
| RAD21 | chr8 | 118743315 | 118743315 | A > G | Change of HDAC2 binding |
| RAD21 | chr8 | 118743315 | 118743315 | A > G | Change of IRF4 binding |
| RAD21 | chr8 | 118743315 | 118743315 | A > G | Change of ZNF569 binding |
| RAD21 | chr8 | 118744058 | 118744058 | T > G | Change of EGR2 binding |
| RAD21 | chr8 | 118744058 | 118744058 | T > G | Change of EGR3 binding |
| RAD21 | chr8 | 118744058 | 118744058 | T > G | Change of EGR4 binding |
| RAD21 | chr8 | 118744058 | 118744058 | T > G | Change of GLI1 binding |
| RAD21 | chr8 | 118744058 | 118744058 | T > G | Change of GLI2 binding |
| RAD50 | chr5 | 131826021 | 131826021 | C > G | Change of EGR4 binding |
| RAD50 | chr5 | 131838005 | 131838005 | G > T | Change of HIVEP3 binding |
| RAD50 | chr5 | 131838005 | 131838005 | G > T | Change of RP58 binding |
| RAD50 | chr5 | 131838005 | 131838005 | G > T | Change of TP63 binding |
| RAD50 | chr5 | 131838005 | 131838005 | G > T | Change of ZFP64 binding |
| RAD50 | chr5 | 131838131 | 131838131 | G > T | Change of NR3C1 binding |
| RAD50 | chr5 | 131838131 | 131838131 | G > T | Change of YYl binding |
| RAD50 | chr5 | 131892812 | 131892812 | T > G | Change of PTF1A binding |
| RAD50 | chr5 | 131892812 | 131892812 | T > G | Change of ZNF721 binding |
| RAD50 | chr5 | 131898457 | 131898457 | G > A | Change of ZNF280B binding |
| RAD50 | chr5 | 132109775 | 132109775 | G > A | Change of ESRRA binding |
| RAD50 | chr5 | 132110041 | 132110041 | G > C | Change of TBX20 binding |
| RAD51C | chr17 | 56012996 | 56012996 | G > C | Change of PAX5 binding |
| RAD51C | chr17 | 56012996 | 56012996 | G > C | Change of SPI1 binding |
| RAD51C | chr17 | 56012996 | 56012996 | G > C | Change of STAT1 binding |
| RAD51C | chr17 | 56405056 | 56405056 | C > T | Change of ZNF93 binding |
| RAD51C | chr17 | 56491423 | 56491423 | T > G | Change of ZNF567 binding |
| RAD51C | chr17 | 56491423 | 56491423 | T > G | Change of ZNF605 binding |
| RAD51C | chr17 | 56565448 | 56565448 | G > C | Change of EGR1 binding |
| RAD51C | chr17 | 56565448 | 56565448 | G > C | Change of EGR4 binding |
| RAD51C | chr17 | 56565448 | 56565448 | G > C | Change of PAX5 binding |
| RAD51C | chr17 | 56565448 | 56565448 | G > C | Change of SP1 binding |
| RAD51C | chr17 | 56565448 | 56565448 | G > C | Change of SP3 binding |
| RAD51C | chr17 | 56565448 | 56565448 | G > C | Change of SP4 binding |
| RAD51C | chr17 | 56565448 | 56565448 | G > C | Change of ZNF236 binding |
| RAD51C | chr17 | 56565448 | 56565448 | G > C | Change of ZNF350 binding |
| RAD51C | chr17 | 56565448 | 56565448 | G > C | Change of ZNF647 binding |
| RAD51C | chr17 | 56686893 | 56686893 | C > T | Change of TP63 binding |
| RAD51C | chr17 | 56686893 | 56686893 | C > T | Change of ZNF302 binding |
| RAD51C | chr17 | 56686893 | 56686893 | C > T | Change of ZNF679 binding |
| RAD51C | chr17 | 56687036 | 56687036 | C > G | Change of AR binding |
| RAD51C | chr17 | 56687036 | 56687036 | C > G | Change of ESR1 binding |
| RAD51C | chr17 | 56687036 | 56687036 | C > G | Change of ESR2 binding |
| RAD51C | chr17 | 56687036 | 56687036 | C > G | Change of ESRRA binding |
| RAD51C | chr17 | 56727563 | 56727563 | C > T | Change of ARNT2 binding |
| RAD51C | chr17 | 56727563 | 56727563 | C > T | Change ofZBTB11 binding |
| RAD51C | chr17 | 56819395 | 56819395 | A > T | Change of NR3C1 binding |
| RAD51C | chr17 | 56819395 | 56819395 | A > T | Change of THAP1 binding |
| RAD51C | chr17 | 56819395 | 56819395 | A > T | Change of YYl binding |
| RAD51C | chr17 | 56841079 | 56841079 | T > A | Change of ZNF136 binding |
| RAD51C | chr17 | 57030283 | 57030283 | C > G | Change of PAX5 binding |
| RAD51C | chr17 | 57030283 | 57030283 | C > G | Change of ZNF430 binding |
| RAD51C | chr17 | 57030283 | 57030283 | C > G | Change of ZNF658B binding |
| RAD51C | chr17 | 57030283 | 57030283 | C > G | Change of ZNF658 binding |
| RAD51C | chr17 | 57030283 | 57030283 | C > G | Change of ZNF709 binding |
| RAD51C | chr17 | 57232982 | 57232982 | G > C | Change of GFIl binding |
| RAD51C | chr17 | 57232982 | 57232982 | G > C | Change of NFYB binding |
| RAD51C | chr17 | 57287378 | 57287378 | A > C | Change of ELK1 binding |
| RAD51C | chr17 | 57288400 | 57288400 | C > A | Change of ZNF493 binding |
| RAD51C | chr17 | 57923238 | 57923238 | A > C | Change of PPARG binding |
| RAD51C | chr17 | 57923238 | 57923238 | A > C | Change of RXRA binding |
| RAD51C | chr17 | 57923418 | 57923418 | G > C | Change of JUN binding |
| RALY | chr20 | 32605327 | 32605327 | G > C | |
| RALY | chr20 | 32610401 | 32610401 | C > T | |
| RALY | chr20 | 32648738 | 32648738 | G > A | |
| RALY | chr20 | 32649064 | 32649064 | A > G | |
| RALY | chr20 | 32655741 | 32655741 | G > A | |
| RALY | chr20 | 32656841 | 32656841 | C > G | |
| RALY | chr20 | 32654562 | 32654562 | C > A | |
| RALY | chr20 | 32657378 | 32657378 | C > T | |
| RARG | chr12 | 53614168 | 53614168 | G > A | Change of ZNF263 binding |
| RARG | chr12 | 53614182 | 53614182 | T > G | Change of ZNF263 binding |
| RARG | chr12 | 53615417 | 53615417 | G > A | Change of ZNF263 binding |
| RARG | chr12 | 53689776 | 53689776 | C > T | Change of ZNF263 binding |
| RARG | chr12 | 53765398 | 53765398 | C > T | Change of ZNF263 binding |
| RASA2, | chr3 | 141807137 | 141807137 | C > C | Increasing of enhancer |
| ATP1B3, | | | | | activity |
| TFDP2, | | | | | |
| XRN1, RNF7, | | | | | |
| GK5, ATR | | | | | |
| RB1 | chr13 | 47128005 | 47128005 | G > A | Change ofCTCF binding |
| RB1 | chr13 | 47128005 | 47128005 | G > A | Change of RXRA binding |
| RB1 | chr13 | 47128005 | 47128005 | G > A | Change of ZBTB7B binding |
| RB1 | chr13 | 48611842 | 48611842 | C > A | Change of ELF2 binding |
| RB1 | chr13 | 48611842 | 48611842 | C > A | Change of ETV7 binding |
| RB1 | chr13 | 48611842 | 48611842 | C > A | Change of FLU binding |
| RB1 | chr13 | 48668171 | 48668171 | T > C | Change of ZNF689 binding |
| RB1 | chr13 | 48669381 | 48669381 | G > T | Change of CTCF binding |
| RB1 | chr13 | 48669381 | 48669381 | G > T | Change of CTCFL binding |
| RB1 | chr13 | 48669381 | 48669381 | G > T | Change of RAD21 binding |
| RB1 | chr13 | 48669381 | 48669381 | G > T | Change of RXRA binding |
| RB1 | chr13 | 48669381 | 48669381 | G > T | Change of SMC3 binding |
| RB1 | chr13 | 48669381 | 48669381 | G > T | Change of YYl binding |
| RB1 | chr13 | 48669381 | 48669381 | G > T | Change of ZFP binding |
| RB1 | chr13 | 48669381 | 48669381 | G > T | Change of ZNF778 binding |
| RB1 | chr13 | 49067569 | 49067569 | G > T | Change of ZAC binding |
| RB1 | chr13 | 50656563 | 50656563 | G > A | Change of DMRT1 binding |
| RB1 | chr13 | 50656563 | 50656563 | G > A | Change of DMRT3 binding |
| RB1 | chr13 | 50656563 | 50656563 | G > A | Change of DMRTA2 binding |
| RBPJ | chr4 | 26085480 | 26085480 | A > G | Increasing of enhancer |
| | | | | | activity |
| RET | NA | NA | NA | Deletion | Loss of enhancer |
| RET | chr10 | 43581812 | 43582711 | | |
| RET | chr10 | 43582056 | 43582056 | C > T | Decreasing of enhancer |
| | | | | | activity |
| RET | chr10 | 43582056 | 43582056 | C > T | Decreasing of enhancer |
| | | | | | activity |
| RET | chr10 | 43582056 | 43582056 | C > T | Loss of SOX10 motif; |
| | | | | | Decreasing of enhancer |
| | | | | | activity |
| RET | chr10 | 43447847 | 43447847 | A > G | Decreasing of enhancer |
| | | | | | activity |
| RET | chr10 | 43447847 | 43447847 | A > G | Loss of RARB motif; |
| | | | | | Decreasing of enhancer |
| | | | | | activity |
| RET | chr10 | 43552895 | 43552895 | C > T | Loss of GATA2 motif; |
| | | | | | Decreasing of enhancer |
| | | | | | activity |
| RFX6 | chr6 | 117210052 | 117210052 | C > T | Gain of HOXB13 motif; |
| | | | | | Increasing of HOXB13 |
| | | | | | binding; Increasing of |
| | | | | | enhancer activity |
| RFX6 | chr6 | 117210052 | 117210052 | C > T | Increasing of HOXB13 |
| | | | | | binding; Increasing of |
| | | | | | enhancer activity |
| RGS1 | chr1 | 192535107 | 192535107 | A > C | |
| RICTOR, FYB, | chr5 | 39397132 | 39397132 | T > A | Increasing of enhancer |
| TTC33, | | | | | activity |
| PRKAA1, | | | | | |
| RPL37, DAB2 | | | | | |
| RP11 | chr1 | 98497176 | 98497176 | A > C | Decreasing of enhancer |
| 490G2.2 | | | | | activity |
| RPS6KA4, | chr11 | 64464085 | 64464085 | T > A | Increasing of enhancer |
| SF1, | | | | | activity |
| SLC22A11, | | | | | |
| SNX15, | | | | | |
| MAP4K2, | | | | | |
| PYGM, | | | | | |
| MEN1, | | | | | |
| NRXN2, | | | | | |
| RASGRP2, | | | | | |
| EHD1, ZFPL1, | | | | | |
| ATG2A, | | | | | |
| ARL2, | | | | | |
| TRMT112 | | | | | |
| RSBN1L, | chr7 | 77416439 | 77416439 | T > C | Increasing of enhancer |
| TMEM60, | | | | | activity |
| PTPN12, | | | | | |
| PION, PHTF2 | | | | | |
| SARDH | chr9 | 136536633 | 136536633 | A > G | |
| SCAMP3, | chr1 | 155194980 | 155194980 | T > T | Increasing of enhancer |
| RUSC1, | | | | | activity |
| FAM189B, | | | | | |
| ADAR, | | | | | |
| LENEP, GBA, | | | | | |
| FLAD1, CLK2, | | | | | |
| ZBTB7B, | | | | | |
| FDPS, MUC1, | | | | | |
| C1orf104, | | | | | |
| MIR555, | | | | | |
| ADAM15, | | | | | |
| HCN3, | | | | | |
| YY1AP1, | | | | | |
| PKLR, DAP3, | | | | | |
| DPM3, | | | | | |
| EFNA1, | | | | | |
| RAG1AP1, | | | | | |
| THBS3, | | | | | |
| KRTCAP2, | | | | | |
| TRIM46, | | | | | |
| CKS1B, | | | | | |
| EFNA3, | | | | | |
| LOC645676, | | | | | |
| ASHIL, | | | | | |
| MSTO1, | | | | | |
| GBAP, | | | | | |
| EFNA4, | | | | | |
| PBXIP1, | | | | | |
| PMVK, | | | | | |
| MTX1, | | | | | |
| PYGO2, SHC1 | | | | | |
| SCG2 | chr2 | 224466344 | 224466344 | C > T | Increasing of enhancer |
| | | | | | activity |
| SCGB3A2 | chr5 | 147258162 | 147258162 | G > A | Decreasing of enhancer |
| | | | | | activity |
| SDCCAG8 | chr1 | 243639859 | 243639859 | A > G | Decreasing of enhancer |
| | | | | | activity |
| SERCA2B | chr12 | 110718411 | 110718411 | G > T | Decreasing of enhancer |
| | | | | | activity |
| SERPINC1 | chr1 | 173886568 | 173886568 | G > C | Decreasing of enhancer |
| | | | | | activity |
| SHF, | chr15 | 45641225 | 45641225 | C > C | Increasing of enhancer |
| SPATA5L1, | | | | | activity |
| MIR147B, | | | | | |
| C15orf21, | | | | | |
| SLC30A4, | | | | | |
| SQRDL, | | | | | |
| SLC28A2, | | | | | |
| GATM, SORD | | | | | |
| SHH | chr7 | 156143386 | 156732204 | Duplication | Gain of enhancer |
| SHH | chr7 | 156241020 | 156619399 | Duplication | Gain of enhancer |
| SHH | chr7 | 156241020 | 156677759 | Duplication | Gain of enhancer |
| SHH | chr7 | 156241020 | 156699998 | Duplication | Gain of enhancer |
| SHH | chr7 | 156354085 | 156619399 | Duplication | Gain of enhancer |
| SHH | chr7 | 156354085 | 156687613 | Duplication | Gain of enhancer |
| SHH | chr7 | 156368541 | 156661877 | Duplication | Gain of enhancer |
| SHH | chr7 | 156539605 | 156699998 | Duplication | Gain of enhancer |
| SHH | chr7 | 156547469 | 156644074 | Duplication | Gain of enhancer |
| SHH | chr7 | 156572751 | 156661877 | Duplication | Gain of enhancer |
| SHH | NA | NA | NA | t(5, 7) (q11, q36) | |
| SHH | NA | NA | NA | inv(7) | Enhancer hijacking; Loss of |
| | | | | (q22.1; q36.3) | intrinsic enhancer; Gain of |
| | | | | | new enhancer |
| SHH | chr1 | 106934 | 106934 | | Gain of enhancer |
| SHH | chr7 | 156583831 | 156583831 | A > G | Gain of ELK4; REL; GMEB1; |
| | | | | | KLF3; ELF3; ELK1; ETV1 |
| | | | | | binding; Increasing of |
| | | | | | enhancer activity |
| SHH | chr7 | 156583831 | 156583831 | A > G | |
| SHH | chr7 | 156583949 | 156583949 | C > G | Gain of REL; GMEB1; KLF3 |
| | | | | | binding; Increasing of |
| | | | | | enhancer activity |
| SHH | chr7 | 156583949 | 156583949 | C > G | |
| SHH | chr7 | 156583951 | 156583951 | C > T | Gain of GMEB1 binding; |
| | | | | | Increasing of enhancer |
| | | | | | activity |
| SHH | chr7 | 156584107 | 156584107 | T > G | Gain of HOXC10; CDX2 |
| | | | | | binding; Increasing of |
| | | | | | enhancer activity |
| SHH | chr7 | 156584107 | 156584107 | T > G | |
| SHH | chr7 | 156584166 | 156584166 | G > A | Gain of HOXD10 binding; |
| | | | | | Increasing of enhancer |
| | | | | | activity |
| SHH | chr7 | 156584166 | 156584166 | G > C | Gain of HOXD10 binding; |
| | | | | | Increasing of enhancer |
| | | | | | activity |
| SHH | chr7 | 156584166 | 156584166 | G > C | |
| SHH | chr7 | 156584166 | 156584166 | G > A | |
| SHH | chr7 | 156584174 | 156584174 | C > T | Increasing of enhancer |
| | | | | | activity |
| SHH | chr7 | 156584174 | 156584174 | C > T | |
| SHH | chr7 | 156584236 | 156584236 | T > G | |
| SHH | chr7 | 156584241 | 156584241 | T > C | Gain of TCF21; MSC; BNC1 |
| | | | | | binding; Increasing of |
| | | | | | enhancer activity |
| SHH | chr7 | 156584241 | 156584241 | T > C | |
| SHH | chr7 | 156584266 | 156584266 | A > T | |
| SHH | chr7 | 156584273 | 156584273 | G > A | |
| SHH | chr7 | 156584275 | 156584275 | T > C | |
| SHH | chr7 | 156584465 | 156584465 | C > G | Gain of TFAP2A; TFAP2E; |
| | | | | | TFAP2B binding; Increasing of |
| | | | | | enhancer activity |
| SHH | chr7 | 156584465 | 156584465 | C > G | |
| SHOX | NA | NA | NA | Deletion | Loss of enhancer |
| SHOX | NA | NA | NA | Deletion | Loss of enhancer |
| SHOX | chrX | 398357 | 835529 | Amplification | Gain of enhancer |
| SIX6 | chr14 | 60974449 | 60974449 | A > G | Gain of enhancer activity |
| SIX6 | chr14 | 60974427 | 60974430 | | Loss of enhancer |
| SLC14A2, | chr18 | 43187130 | 43187130 | A > A | Increasing of enhancer |
| SLC14A1 | | | | | activity |
| SLC1A2 | chr11 | 35440976 | 35440976 | T > G | Gain of WT1; SMAD4 binding; |
| | | | | | Decreasing of enhancer |
| | | | | | activity |
| SLC25A37, | chr8 | 23751151 | 23751151 | C > T | Increasing of enhancer |
| NKX3-1, | | | | | activity |
| ENTPD4, | | | | | |
| STC1, | | | | | |
| ADAM7 | | | | | |
| SLC26A4 | chr7 | 107301201 | 107301201 | T > C | Gain of GMEB1; SMAD4 |
| | | | | | binding; Decreasing of |
| | | | | | enhancer activity |
| SLC37A4 | chr11 | 118900338 | 118900338 | G > A | Decreasing of enhancer |
| | | | | | activity |
| SLC4A7 | chr3 | 27548900 | 27548900 | C > T | Increasing of c-Fos binding; |
| | | | | | Increasing of enhancer |
| | | | | | activity |
| SLC6A4 | chr17 | 28564170 | 28564170 | A > G | Increasing of enhancer |
| | | | | | activity |
| SMAD2 | chr18 | 45446307 | 45446307 | A > G | Decreasing of enhancer |
| | | | | | activity |
| SMAD3 | chr15 | 67442596 | 67442596 | C > T | Loss of AP-1 motif; |
| | | | | | Decreasing of enhancer |
| | | | | | activity |
| SMAD7 | chr18 | 46449565 | 46449565 | C > G | Decreasing of enhancer |
| | | | | | activity |
| SMAD7 | chr18 | 46448805 | 46448805 | C > T | Decreasing of enhancer |
| | | | | | activity |
| SMAD7 | chr18 | 46448819 | 46448819 | G > C | Decreasing of enhancer |
| | | | | | activity |
| SMAD7 | chr18 | 46449111 | 46449111 | T > C | Decreasing of enhancer |
| | | | | | activity |
| SMARCA1 | | NA | NA | | Gain of enhancer |
| SMARCA1 | | NA | NA | | Gain of enhancer |
| SMARCA1 | | NA | NA | | Gain of enhancer |
| SMARCA1 | | NA | NA | | Gain of enhancer |
| SMARCA1 | | NA | NA | | Gain of enhancer |
| SMARCA1 | | NA | NA | | Gain of enhancer |
| SMARCA1 | | NA | NA | | Gain of enhancer |
| SMARCA1 | | NA | NA | | Gain of enhancer |
| SMARCA1 | | NA | NA | | Gain of enhancer |
| SMARCA1 | | NA | NA | | Gain of enhancer |
| SMARCA1 | | NA | NA | | Gain of enhancer |
| SMARCA1 | | NA | NA | | Gain of enhancer |
| SMARCA1 | | NA | NA | | Gain of enhancer |
| SMARCA1 | | NA | NA | | Gain of enhancer |
| SMARCD1 | chr12 | 50451445 | 50451445 | C > G | Change of EGR4 binding |
| SMARCD1 | chr12 | 50451445 | 50451445 | C > G | Change of HDAC2 binding |
| SMARCD1 | chr12 | 50451445 | 50451445 | C > G | Change of REST binding |
| SMARCD1 | chr12 | 50451445 | 50451445 | C > G | Change of SP4 binding |
| SNCA | chr4 | 90674431 | 90674431 | G > A | Increasing of EMX2; NKX6-1 |
| | | | | | binding; Increasing of |
| | | | | | enhancer activity |
| SNHG5 | chr6 | 86246709 | 86246709 | A > C | Decreasing of enhancer |
| | | | | | activity |
| SNHG5 | chr6 | 86246709 | 86246709 | A > C | Decreasing of enhancer |
| | | | | | activity |
| SNHG5 | chr6 | 86223316 | 86223316 | C > T | Decreasing of enhancer |
| | | | | | activity |
| SNHG5 | chr6 | 86223316 | 86223316 | C > T | Decreasing of enhancer |
| | | | | | activity |
| SNHG5 | chr6 | 86258614 | 86258614 | A > G | Decreasing of enhancer |
| | | | | | activity |
| SNHG5 | chr6 | 86258614 | 86258614 | A > G | Decreasing of enhancer |
| | | | | | activity |
| SNHG5 | chr6 | 86224053 | 86224053 | G > T | Decreasing of enhancer |
| | | | | | activity |
| SNHG5 | chr6 | 86224053 | 86224053 | G > T | Decreasing of enhancer |
| | | | | | activity |
| SNHG5 | chr6 | 86205323 | 86205323 | G > T | Decreasing of enhancer |
| | | | | | activity |
| SNHG5 | chr6 | 86205323 | 86205323 | G > T | Decreasing of enhancer |
| | | | | | activity |
| SNHG5 | chr6 | 86247799 | 86247799 | C > T | Decreasing of enhancer |
| | | | | | activity |
| SNHG5 | chr6 | 86247799 | 86247799 | C > T | Decreasing of enhancer |
| | | | | | activity |
| SOD2, | chr6 | 160668389 | 160668389 | A > G | Increasing of enhancer |
| SLC22A2, | | | | | activity |
| SLC22A1 | | | | | |
| SORT1 | chr1 | 109817590 | 109817590 | G > T | Gain of C/EBP motif; |
| | | | | | Increasing of enhancer |
| | | | | | activity |
| SOX10 | chr22 | 38412215 | 38412215 | G > A | Loss of TFAP2A motif; |
| | | | | | Decreasing of enhancer |
| | | | | | activity |
| SOX10 | chr22 | 38412781 | 38412781 | G > C | Decreasing of SOX10 binding; |
| | | | | | Decreasing of enhancer |
| | | | | | activity |
| SOX10 | chr22 | 38412423 | 38412423 | C > T | |
| SOX10 | chr22 | 38412323 | 38412323 | C > T | |
| SOX10 | chr22 | 38412402 | 38412402 | C > T | |
| SOX9 | chr17 | 68676303 | 68676303 | T > C | Gain of KLF3; HES1; HEY1; |
| | | | | | HEY2; HES5 binding; |
| | | | | | Decreasing of enhancer |
| | | | | | activity |
| SOX9 | chr17 | 68698977 | 68698977 | C > T | Loss of OCT1 motif; |
| | | | | | Decreasing of enhancer |
| | | | | | activity |
| SOX9 | chr17 | 68735530 | 68735530 | G > A | Decreasing of enhancer |
| | | | | | activity |
| SOX9 | chr17 | 68747322 | 68747322 | T > C | Loss of NFAT motif; |
| | | | | | Decreasing of enhancer |
| | | | | | activity |
| SOX9 | chr17 | 68772750 | 68772750 | G > A | Loss of CDP motif; Decreasing |
| | | | | | of enhancer activity |
| SOX9 | chr17 | 69706044 | 69706044 | A > C | Decreasing of enhancer |
| | | | | | activity |
| SOX9 | chr17 | 69108655 | 69108655 | A > G | Gain of AP-1 motif; Loss of |
| | | | | | FOXA1 |
| | | | | | motif; Increasing of |
| | | | | | enhancer activity |
| SOX9 | chr17 | 69108753 | 69108753 | T > G | Increasing of enhancer |
| | | | | | activity |
| SOX9 | chr17 | 69107816 | 69107816 | G > A | Gain of androgen receptor |
| | | | | | motif; Increasing of enhancer |
| | | | | | activity |
| SPCS1 | chr3 | 52804487 | 52804487 | T > C | Decreasing of enhancer |
| | | | | | activity |
| SPINK1 | chr5 | 147211221 | 147211221 | G > A | Gain of GMEB1; SMAD4 |
| | | | | | binding; Increasing of |
| | | | | | enhancer activity |
| SPINK1 | chr5 | 147211355 | 147211355 | C > T | Gain of MYF6 binding; |
| | | | | | Increasing of enhancer |
| | | | | | activity |
| SRCAP | chr16 | 30669784 | 30669784 | G > C | Change of ZNF263 binding |
| SRCAP | chr16 | 30723097 | 30723097 | G > A | Change of ZNF263 binding |
| SRCAP | chr16 | 30747922 | 30747922 | C > T | Change of ZNF263 binding |
| SRCAP | chr16 | 30753142 | 30753142 | C > T | Change of SP1 binding |
| SRCAP | chr16 | 30753142 | 30753142 | C > T | Change of SP1 binding |
| STARD10 | chr11 | 72470916 | 72470916 | Deletion | Gain of a GTTT repeat; |
| | | | | | Decreasing of enhancer |
| | | | | | activity |
| STAT3 | chr17 | 40507980 | 40507980 | A > C | |
| STUB1 | | NA | NA | | Gain of enhancer |
| STUB1 | | NA | NA | | Gain of enhancer |
| STUB1 | | NA | NA | | Gain of enhancer |
| STUB1 | | NA | NA | | Gain of enhancer |
| STUB1 | | NA | NA | | Gain of enhancer |
| STUB1 | | NA | NA | | Gain of enhancer |
| STUB1 | | NA | NA | | Gain of enhancer |
| STUB1 | | NA | NA | | Gain of enhancer |
| STUB1 | | NA | NA | | Gain of enhancer |
| STUB1 | | NA | NA | | Gain of enhancer |
| STUB1 | | NA | NA | | Gain of enhancer |
| STUB1 | | NA | NA | | Gain of enhancer |
| STUB1 | | NA | NA | | Gain of enhancer |
| SULF2 | chr20 | 46602388 | 46602388 | C > T | Change of EGR4 binding |
| SULF2 | chr20 | 46602388 | 46602388 | C > T | Change of MAZ binding |
| SULF2 | chr20 | 46602388 | 46602388 | C > T | Change of SP1 binding |
| SULF2 | chr20 | 46602388 | 46602388 | C > T | Change of SP3 binding |
| SULF2 | chr20 | 46602388 | 46602388 | C > T | Change of SP4 binding |
| SULF2 | chr20 | 46602388 | 46602388 | C > T | Change of ZBTB7B binding |
| SULF2 | chr20 | 46602388 | 46602388 | C > T | Change of ZNF263 binding |
| SULF2 | chr20 | 46602388 | 46602388 | C > T | Change of ZNF658B binding |
| SULF2 | chr20 | 46603160 | 46603160 | C > T | Change of ZNF263 binding |
| SULF2 | chr20 | 46618545 | 46618545 | C > G | Change of ZNF263 binding |
| SULF2 | chr20 | 46618545 | 46618545 | C > G | Change of ZNF263 binding |
| SULF2 | chr20 | 52748710 | 52748710 | C > T | Change of ZNF263 binding |
| SULF2 | chr20 | 46602388 | 46602388 | C > T | Change of EGR4 binding |
| SULF2 | chr20 | 46602388 | 46602388 | C > T | Change of MAZ binding |
| SULF2 | chr20 | 46602388 | 46602388 | C > T | Change of SP1 binding |
| SULF2 | chr20 | 46602388 | 46602388 | C > T | Change of SP3 binding |
| SULF2 | chr20 | 46602388 | 46602388 | C > T | Change of SP4 binding |
| SULF2 | chr20 | 46602388 | 46602388 | C > T | Change of ZBTB7B binding |
| SULF2 | chr20 | 46602388 | 46602388 | C > T | Change of ZNF263 binding |
| SULF2 | chr20 | 46602388 | 46602388 | C > T | Change of ZNF658B binding |
| SULF2 | chr20 | 46472698 | 46472698 | T > G | Change of HOXC5 binding |
| SULF2 | chr20 | 46472698 | 46472698 | T > G | Change of MAZ binding |
| SULF2 | chr20 | 46472698 | 46472698 | T > G | Change of SP1 binding |
| SULF2 | chr20 | 46472698 | 46472698 | T > G | Change of ZBTB7B binding |
| SULF2 | chr20 | 46472698 | 46472698 | T > G | Change of ZNF263 binding |
| SULF2 | chr20 | 46472698 | 46472698 | T > G | Change of ZNF658B binding |
| SULF2 | chr20 | 46308627 | 46308627 | T > G | Change of EGR4 binding |
| SULF2 | chr20 | 46308627 | 46308627 | T > G | Change of MAZ binding |
| SULF2 | chr20 | 46308627 | 46308627 | T > G | Change of SP1 binding |
| SULF2 | chr20 | 46308627 | 46308627 | T > G | Change of SP3 binding |
| SULF2 | chr20 | 46308627 | 46308627 | T > G | Change of SP4 binding |
| SULF2 | chr20 | 46308627 | 46308627 | T > G | Change of ZBTB7B binding |
| SULF2 | chr20 | 46308627 | 46308627 | T > G | Change of ZNF263 binding |
| SULF2 | chr20 | 46308627 | 46308627 | T > G | Change of ZNF658B binding |
| SULF2 | chr20 | 46831426 | 46831426 | G > A | Change of EGR4 binding |
| SULF2 | chr20 | 46831426 | 46831426 | G > A | Change of SP3 binding |
| SULF2 | chr20 | 46831426 | 46831426 | G > A | Change of SP4 binding |
| SULF2 | chr20 | 46831426 | 46831426 | G > A | Change of ZNF263 binding |
| SULF2 | chr20 | 46779797 | 46779797 | G > C | Change of HOXC5 binding |
| SULF2 | chr20 | 46793997 | 46793997 | G > T | Change of HOXC5 binding |
| SULF2 | chr20 | 46800093 | 46800093 | G > A | Change of SP1 binding |
| SULF2 | chr20 | 46800093 | 46800093 | G > A | Change of SP4 binding |
| SULF2 | chr20 | 46800093 | 46800093 | G > A | Change of SP1 binding |
| SULF2 | chr20 | 46800093 | 46800093 | G > A | Change of SP4 binding |
| SULF2 | chr20 | 47364914 | 47364914 | G > C | Change of SP1 binding |
| SULF2 | chr20 | 47366010 | 47366010 | G > C | Change of SP1 binding |
| SULF2 | chr20 | 46688376 | 46688376 | C > G | Change of SP1 binding |
| SULF2 | chr20 | 45989659 | 45989659 | G > A | Change of SP1 binding |
| SULF2 | chr20 | 46182305 | 46182305 | A > C | Change of EGR4 binding |
| SULF2 | chr20 | 46182305 | 46182305 | A > C | Change of MAZ binding |
| SULF2 | chr20 | 46182305 | 46182305 | A > C | Change of SP3 binding |
| SULF2 | chr20 | 46182305 | 46182305 | A > C | Change of SP4 binding |
| SULF2 | chr20 | 46182305 | 46182305 | A > C | Change of ZBTB7B binding |
| SULF2 | chr20 | 52703188 | 52703188 | G > T | Change of ZNF658B binding |
| SULF2 | chr20 | 52703188 | 52703188 | G > T | Change of ZNF658B binding |
| SULF2 | chr20 | 46779797 | 46779797 | G > C | Change of HOXC5 binding |
| SULF2 | chr20 | 46779797 | 46779797 | G > C | Change of HOXC5 binding |
| SULF2 | chr20 | 46710095 | 46710095 | A > T | Change of HOXC5 binding |
| TAGAP | chr6 | 159521657 | 159564248 | Duplication | Gain of enhancer |
| TAGAP | chr6 | 159521657 | 159564248 | Duplication | Gain of enhancer |
| TAGAP | chr6 | 159521657 | 159564248 | Duplication | Gain of enhancer |
| TAGAP | chr6 | 159521657 | 159564248 | Duplication | Gain of enhancer |
| TAGAP | chr6 | 159521657 | 159564248 | Duplication | Gain of enhancer |
| TAGAP | chr6 | 159521657 | 159564248 | Duplication | Gain of enhancer |
| TAGAP | chr6 | 159521657 | 159564248 | Duplication | Gain of enhancer |
| TAGAP | chr6 | 159521657 | 159564248 | Tandem | Gain of enhancer |
| | | | | duplication | |
| TAL1 | chr1 | 47704954 | 47704983 | | Gain of MYB; CBP; RUNX1; |
| | | | | | GATA3 and TAL1 binding; |
| | | | | | Gain of enhancer |
| TAL1 | chr1 | 94555832 | 211935484 | Deletion | Gain of enhancer |
| TBL1X | | NA | NA | | Gain of enhancer |
| TBL1X | | NA | NA | | Gain of enhancer |
| TBL1X | | NA | NA | | Gain of enhancer |
| TBL1X | | NA | NA | | Gain of enhancer |
| TBL1X | | NA | NA | | Gain of enhancer |
| TBL1X | | NA | NA | | Gain of enhancer |
| TBL1X | | NA | NA | | Gain of enhancer |
| TBL1X | | NA | NA | | Gain of enhancer |
| TBL1X | | NA | NA | | Gain of enhancer |
| TBL1X | | NA | NA | | Gain of enhancer |
| TBL1X | | NA | NA | | Gain of enhancer |
| TBL1X | | NA | NA | | Gain of enhancer |
| TBL1X | | NA | NA | | Gain of enhancer |
| TBL1X | | NA | NA | | Gain of enhancer |
| TBL1X | | NA | NA | | Gain of enhancer |
| TBX1 | chr22 | 19743578 | 19743578 | C > T | Increasing of enhancer |
| | | | | | activity |
| TBX3 | chr12 | 115112467 | 115112467 | T > C | Change of E2F1 binding |
| TBX3 | chr12 | 115112467 | 115112467 | T > C | Change of REST binding |
| TBX3 | chr12 | 115112467 | 115112467 | T > C | Change of ZBTB7B binding |
| TBX3 | chr12 | 115112467 | 115112467 | T > C | Change of ZNF236 binding |
| TBX4, BCAS3, | chr17 | 59239221 | 59239221 | A > A | Increasing of enhancer |
| TBX2, NACA2 | | | | | activity |
| TBX5 | chr12 | 114704515 | 114704515 | G > T | Loss of enhancer |
| TBX5 | chr12 | 114704515 | 114704515 | G > T | Loss of enhancer |
| TCF21 | chr6 | 134214525 | 134214525 | G > C | Increasing of AP-1 binding; |
| | | | | | Increasing of enhancer |
| | | | | | activity |
| TCF7L2 | chr10 | 114808902 | 114808902 | G > T | Increasing of enhancer |
| | | | | | activity |
| TCF7L2 | chr10 | 114758349 | 114758349 | C > T | Increasing of enhancer |
| | | | | | activity |
| TCF7L2 | chr10 | 114758349 | 114758349 | C > T | Loss of HMGB1 binding; Gain |
| | | | | | of enhancer activity |
| TERT | chr5 | 1325104 | 1345104 | | Gain of enhancer |
| TERT | chr5 | 1325104 | 1345104 | | Gain of enhancer |
| TERT | chr5 | 1325104 | 1345104 | | Gain of enhancer |
| TERT | chr5 | 1286516 | 1286516 | T > G | Increasing of enhancer |
| | | | | | activity |
| TERT | chr5 | 1286516 | 1286516 | A > C | Increasing of enhancer |
| | | | | | activity |
| TERT | chr5 | 1287194 | 1287194 | T > C | Loss of Snail1 motif; |
| | | | | | Decreasing Snail1 binding; |
| | | | | | Increasing of enhancer |
| | | | | | activity |
| TERT | | NA | NA | | Gain of enhancer |
| TERT | | NA | NA | | Gain of enhancer |
| TERT | | NA | NA | | Gain of enhancer |
| TERT | | NA | NA | | Gain of enhancer |
| TERT | | NA | NA | | Gain of enhancer |
| TERT | | NA | NA | | Gain of enhancer |
| TERT | | NA | NA | | Gain of enhancer |
| TERT | | NA | NA | | Gain of enhancer |
| TERT | | NA | NA | | Gain of enhancer |
| TERT | | NA | NA | | Gain of enhancer |
| TERT | | NA | NA | | Gain of enhancer |
| TERT | | NA | NA | | Gain of enhancer |
| TFF1 | chr21 | 43796913 | 43796913 | A > T | Loss of Rad21 binding; |
| | | | | | Loss of |
| | | | | | enhancer |
| TFF1 | chr21 | 43796494 | 43796494 | C > G | Loss of ER-alpha binding; |
| | | | | | Loss |
| | | | | | of enhancer |
| TH | chr11 | 2193085 | 2193085 | A > T | Decreasing of enhancer |
| | | | | | activity |
| TH | chr11 | 2193086 | 2193086 | C > T | Decreasing of enhancer |
| | | | | | activity |
| TH | chr11 | 2193087 | 2193087 | G > A | Decreasing of enhancer |
| | | | | | activity |
| THRB | chr3 | 24204984 | 24204984 | C > T | |
| TNF | chr6 | 31542308 | 31542308 | T > C | |
| TNF | chr6 | 31542476 | 31542476 | C > A | |
| TNFAIP3 | chr6 | 138196066 | 138196066 | TT > A | |
| TNFAIP3 | chr6 | 138196066 | 138196066 | T > G | Loss of NF-CE∫B binding; |
| | | | | | Loss |
| | | | | | of enhancer activity |
| TNFRSF10A | chr8 | 23082971 | 23082971 | G > T | Gain of ZDHHC7; PLAGL1 |
| | | | | | binding; Decreasing of |
| | | | | | enhancer activity |
| TNFRSF1A | chr12 | 6440009 | 6440009 | T > C | |
| TOX2 | chr20 | 42568164 | 42568164 | C > T | |
| TOX3 | chr16 | 52586341 | 52586341 | A > G | Gain of TOX3 motif and CHD9 |
| | | | | | motif; Decreasing of |
| | | | | | enhancer activity |
| TOX3 | chr16 | 52599188 | 52599188 | C > T | Gain of FOXA1 binding; |
| | | | | | Gain |
| | | | | | of enhancer activity |
| TP53 | chr17 | 6938079 | 6938079 | A > G | Change of ZNF471 binding |
| TP53 | chr17 | 6939213 | 6939213 | C > A | Change of PAX5 binding |
| TP53 | chr17 | 6939213 | 6939213 | C > A | Change of ZNF672 binding |
| TP53 | chr17 | 7465409 | 7465409 | T > G | Change of GTF3C2 binding |
| TP53 | chr17 | 7465409 | 7465409 | T > G | Change of SP4 binding |
| TP53 | chr17 | 7465409 | 7465409 | T > G | Change ofZBTB11 binding |
| TP53 | chr17 | 7465409 | 7465409 | T > G | Change of ZBTB7B binding |
| TP53 | chr17 | 7465409 | 7465409 | T > G | Change of ZIC3 binding |
| TP53 | chr17 | 7465409 | 7465409 | T > G | Change of ZNF345 binding |
| TP53 | chr17 | 7534110 | 7534110 | G > A | Change of ZBTB38 binding |
| TP53 | chr17 | 7577498 | 7577498 | C > A | Change of RARB binding |
| TP53 | chr17 | 7577498 | 7577498 | C > A | Change of THRA binding |
| TP53 | chr17 | 7577580 | 7577580 | T > C | Change of SMARCC2 binding |
| TP53 | chr17 | 7577580 | 7577580 | T > C | Change of ZNF440 binding |
| TP53 | chr17 | 7578271 | 7578271 | T > C | Change of SP1 binding |
| TP53 | chr17 | 7578394 | 7578394 | T > C | Change of ZBTB41 binding |
| TP53 | chr17 | 7578394 | 7578394 | T > C | Change of ZNF180 binding |
| TP53 | chr17 | 7578394 | 7578394 | T > C | Change of ZNF436 binding |
| TP53 | chr17 | 7578394 | 7578394 | T > C | Change of ZNF880 binding |
| TP53 | chr17 | 7578478 | 7578478 | G > C | Change of EGR1 binding |
| TP53 | chr17 | 7578478 | 7578478 | G > C | Change of KLF16 binding |
| TP53 | chr17 | 7578478 | 7578478 | G > C | Change of POLR3A binding |
| TP53 | chr17 | 7578478 | 7578478 | G > C | Change of SP3 binding |
| TP53 | chr17 | 7578478 | 7578478 | G > C | Change of SP4 binding |
| TP53 | chr17 | 7578478 | 7578478 | G > C | Change of TBX21 binding |
| TP53 | chr17 | 7578478 | 7578478 | G > C | Change of WT1 binding |
| TP53 | chr17 | 7578478 | 7578478 | G > C | Change of ZFP2 binding |
| TP53 | chr17 | 7578478 | 7578478 | G > C | Change of ZNF729 binding |
| TP53 | chr17 | 7578796 | 7578796 | C > A | Change of CTCF binding |
| TP53 | chr17 | 7578796 | 7578796 | C > A | Change of RAD21 binding |
| TP53 | chr17 | 7578796 | 7578796 | C > A | Change of RXRA binding |
| TP53 | chr17 | 7578796 | 7578796 | C > A | Change of ZNF92 binding |
| TP53 | chr17 | 7578947 | 7578947 | C > T | Change of NHLH2 binding |
| TP53 | chr17 | 7578947 | 7578947 | C > T | Change of SREBF1 binding |
| TP53 | chr17 | 7579313 | 7579313 | G > C | Change of POU2F1 binding |
| TP53 | chr17 | 7579529 | 7579529 | C > T | Change of SOX9 binding |
| TP53 | chr17 | 7621215 | 7621215 | T > G | Change of ZN337 binding |
| TP53 | chr17 | 8023124 | 8023124 | C > T | Change of E2F4 binding |
| TP53 | chr17 | 8023124 | 8023124 | C > T | Change of HSF1 binding |
| TP53 | chr17 | 8023124 | 8023124 | C > T | Change of RBAK binding |
| TP53 | chr17 | 8026793 | 8026793 | C > T | Change of MAX binding |
| TP53 | chr17 | 8026793 | 8026793 | C > T | Change of ZNF790 binding |
| TPD52L3, | chr9 | 6365683 | 6365683 | A > C | Decreasing of enhancer |
| TMED6, | | | | | activity |
| JMJD2C | | | | | |
| TPTE | chr21 | 10816017 | 10816017 | G > C | Change of ZNF616 binding |
| TPTE | chr21 | 10816529 | 10816529 | A > G | Change of ZNF616 binding |
| TPTE | chr21 | 10813552 | 10813552 | G > A | Change of ZNF616 binding |
| TPTE | chr21 | 10814023 | 10814023 | A > G | Change of ZNF616 binding |
| TPTE | chr21 | 10814025 | 10814025 | G > T | Change of ZNF616 binding |
| TPTE | chr21 | 10814061 | 10814061 | G > T | Change of ZNF616 binding |
| TPTE | chr21 | 10814155 | 10814155 | G > T | Change of ZNF616 binding |
| TPTE | chr21 | 10814716 | 10814716 | G > T | Change of ZNF616 binding |
| TPTE | chr21 | 10832792 | 10832792 | G > T | Change of ZNF616 binding |
| TPTE | chr21 | 10833977 | 10833977 | G > A | Change of ZNF616 binding |
| TPTE | chr21 | 10833990 | 10833990 | T > C | Change of ZNF616 binding |
| TPTE | chr21 | 10834357 | 10834357 | G > A | Change of ZNF616 binding |
| TPTE | chr21 | 10851202 | 10851202 | A > G | Change of ZNF616 binding |
| TPTE | chr21 | 10853066 | 10853066 | T > G | Change of ZNF616 binding |
| TPTE | chr21 | 10820450 | 10820450 | G > C | Change of ZNF616 binding |
| TPTE | chr21 | 10796504 | 10796504 | G > T | Change of ZNF616 binding |
| TPTE | chr21 | 10849949 | 10849949 | G > A | Change of ZNF616 binding |
| TPTE | chr21 | 10850097 | 10850097 | T > A | Change of ZNF616 binding |
| TPTE | chr21 | 10851202 | 10851202 | A > G | Change of ZNF616 binding |
| TPTE | chr21 | 10845180 | 10845180 | G > T | Change of ZNF616 binding |
| TPTE | chr21 | 10845749 | 10845749 | G > C | Change of ZNF616 binding |
| TPTE | chr21 | 10780021 | 10780021 | G > T | Change of ZNF616 binding |
| TPTE | chr21 | 10830716 | 10830716 | G > A | Change of ZNF616 binding |
| TPTE | chr21 | 10831264 | 10831264 | T > C | Change of ZNF616 binding |
| TRABD | chr22 | 50627834 | 50627834 | C > T | Change of PLAG1 binding |
| TRABD | chr22 | 50714820 | 50714820 | T > A | Change of PLAG1 binding |
| TRABD | chr22 | 50639543 | 50639543 | G > A | Change of PLAG1 binding |
| TRABD | chr22 | 50608627 | 50608627 | C > G | Change of PLAG1 binding |
| TRIM27 | chr6 | 28949254 | 28949254 | A > C | Decreasing of enhancer |
| | | | | | activity |
| TRIM27 | chr6 | 28949312 | 28949312 | C > T | Increasing of enhancer |
| | | | | | activity |
| TRIM27 | chr6 | 28949469 | 28949469 | C > A | Increasing of enhancer |
| | | | | | activity |
| TRIM27 | chr6 | 28949509 | 28949509 | G > A | Increasing of enhancer |
| | | | | | activity |
| TRIM27 | chr6 | 28949511 | 28949511 | G > A | Increasing of enhancer |
| | | | | | activity |
| TRIM27 | chr6 | 28949524 | 28949524 | C > T | Increasing of enhancer |
| | | | | | activity |
| TRIM27 | chr6 | 28949535 | 28949535 | C > G | Increasing of enhancer |
| | | | | | activity |
| TRIM27 | chr6 | 28949821 | 28949821 | C > T | Increasing of enhancer |
| | | | | | activity |
| TRIM27 | chr6 | 28949841 | 28949841 | G > A | Increasing of enhancer |
| | | | | | activity |
| TRIM27 | chr6 | 28949944 | 28949944 | C > A | Increasing of enhancer |
| | | | | | activity |
| TRIM27 | chr6 | 28950014 | 28950014 | C > T | Increasing of enhancer |
| | | | | | activity |
| TRIM27 | chr6 | 28950037 | 28950037 | CGGTCAGGGA | Increasing of enhancer |
| | | | | ATGAGGTTTT | activity |
| | | | | TCTGTTTTAA | |
| | | | | CCT | |
| | | | | (SEQ ID | |
| | | | | NO: 480)>- | |
| TRIM27 | chr6 | 28950039 | 28950039 | G > A | Increasing of enhancer |
| | | | | | activity |
| TRIM27 | chr6 | 28950050 | 28950050 | G > A | Decreasing of enhancer |
| | | | | | activity |
| TRIM27 | chr6 | 28950052 | 28950052 | G > A | Increasing of enhancer |
| | | | | | activity |
| TRIM27 | chr6 | 28950056 | 28950056 | T > C | Increasing of enhancer |
| | | | | | activity |
| TRIM27 | chr6 | 28950486 | 28950486 | G > T | Increasing of enhancer |
| | | | | | activity |
| TRIM27 | chr6 | 28950885 | 28950885 | A > G | Increasing of enhancer |
| | | | | | activity |
| TRMT11, | chr6 | 127436064 | 127436064 | C > C | Increasing of enhancer |
| ECHDC1, | | | | | activity |
| HINT3, | | | | | |
| C6orf173, | | | | | |
| RNF146 | | | | | |
| TSC22D3 | | NA | NA | | Gain of enhancer |
| TSC22D3 | | NA | NA | | Gain of enhancer |
| TSC22D3 | | NA | NA | | Gain of enhancer |
| TSC22D3 | | NA | NA | | Gain of enhancer |
| TSC22D3 | | NA | NA | | Gain of enhancer |
| TSC22D3 | | NA | NA | | Gain of enhancer |
| TSC22D3 | | NA | NA | | Gain of enhancer |
| TSC22D3 | | NA | NA | | Gain of enhancer |
| TSC22D3 | | NA | NA | | Gain of enhancer |
| TSC22D3 | | NA | NA | | Gain of enhancer |
| TSLP | chr5 | 110176128 | 110176128 | G > T | Increasing of NHLH1 binding; |
| | | | | | Increasing of enhancer |
| | | | | | activity |
| TSPAN31, | chr12 | 58165085 | 58165085 | G > A | Increasing of enhancer |
| CYP27B1, | | | | | activity |
| TSFM, | | | | | |
| AVIL, | | | | | |
| FAM119B | | | | | |
| TTC24 | chr1 | 156470002 | 156470002 | T > A | Change of SP4 binding |
| TTC24 | chr1 | 156470227 | 156470227 | T > G | Change of SP4 binding |
| TTC24 | chr1 | 156470331 | 156470331 | C > A | Change of SP4 binding |
| TTC24 | chr1 | 156470445 | 156470445 | C > G | Change of SP4 binding |
| TTC24 | chr1 | 156470674 | 156470674 | T > C | Change of SP4 binding |
| TTC24 | chr1 | 156561525 | 156561525 | T > C | Change of SP4 binding |
| TTC24 | chr1 | 156561584 | 156561584 | C > G | Change of SP4 binding |
| TTC24 | chr1 | 156645564 | 156645564 | G > A | Change of SP4 binding |
| TYK2, ICAM3 | chr19 | 10520064 | 10520064 | G > A | |
| TYW5 | chr2 | 200780737 | 200780737 | T > C | Decreasing of enhancer |
| | | | | | activity |
| UCP2 | chr11 | 73694754 | 73694754 | C > T | Gain of PAX5; PAX8 binding; |
| | | | | | Increasing of enhancer |
| | | | | | activity |
| UPK3A | chr22 | 45676678 | 45676678 | A > G | Loss of GATA2 motif; Loss of |
| | | | | | enhancer activity |
| UROS | chr10 | 127505277 | 127505277 | C > T | Gain of ESRRG; NR2F6; |
| | | | | | NR2F2; NR2C2 binding; |
| | | | | | Decreasing of enhancer |
| | | | | | activity |
| USP12 | chr13 | 27523026 | 27544353 | Amplification | Gain of enhancer |
| VPS13C | chr15 | 62391608 | 62391608 | T > C | Decreasing of enhancer |
| | | | | | activity |
| WFS1 | chr4 | 6299940 | 6299940 | C > T | Decreasing of enhancer |
| | | | | | activity |
| WFS1 | chr4 | 6299387 | 6299387 | G > A | Decreasing of enhancer |
| | | | | | activity |
| XBP1 | chr22 | 29196757 | 29196757 | G > C | Gain of ZIC3 binding; |
| | | | | | Decreasing of enhancer |
| | | | | | activity |
| ZFAND3 | chr6 | 37775652 | 37775652 | G > A | Loss of NEUROD1 binding; |
| | | | | | Loss of enhancer |
| ZFAS1 | chr20 | 47905499 | 47905499 | T > G | Gain of JUND motif; |
| | | | | | Increasing of enhancer |
| | | | | | activity |
| ZFP36L1 | chr14 | 69254191 | 69254191 | C > T | |
| ZIP11 | chr17 | 71006512 | 71006512 | A > C | |
| ZIP11 | chr17 | 71012473 | 71012473 | G > C | |
| ZMYND11 | chr10 | 375539 | 375539 | A > G | Change of ZNF274 binding |
| ZMYND11 | chr10 | 127618 | 127618 | A > C | Change of ZNF274 binding |
| ZNF48 | chr16 | 30935409 | 30935409 | C > G | Change of ZNF263 binding |
| ZNF48 | chr16 | 29911922 | 29911922 | G > C | Change of ZNF263 binding |
| ZNF48 | chr16 | 30346156 | 30346156 | A > C | Change of ZNF263 binding |
| ZNF48, | chr16 | 30669784 | 30669784 | G > C | Change of ZNF263 binding |
| SRCAP | | | | | |
| ZNF48, | chr16 | 30346156 | 30346156 | A > C | Change of ZNF263 binding |
| SRCAP | | | | | |
| ZNF512B | chr20 | 62594247 | 62594247 | T > C | Decreasing of enhancer |
| | | | | | activity |
| ZNF517, | chr8 | 145913001 | 145913001 | A > C | Change of ZNF224 binding |
| RPL8 | | | | | |
| ZNF517, | chr8 | 145913120 | 145913120 | A > T | Change of ZNF224 binding |
| RPL8 | | | | | |
| ZNF517, | chr8 | 145913262 | 145913262 | A > T | Change of ZNF224 binding |
| RPL8 | | | | | |
| ZNF596 | chr8 | 580156 | 580156 | C > G | Decreasing of enhancer |
| | | | | | activity |
| ZNF596 | chr8 | 580332 | 580332 | C > T | Decreasing of enhancer |
| | | | | | activity |
| ZNF596 | chr8 | 580370 | 580370 | C > T | Decreasing of enhancer |
| | | | | | activity |
| ZNF596 | chr8 | 580698 | 580698 | G > A | Decreasing of enhancer |
| | | | | | activity |
| ZNF596 | chr8 | 581293 | 581293 | G > C | Decreasing of enhancer |
| | | | | | activity |

### Methods of Reprogramming Cells

The present methods can include using SHAPE to alter expression of cell fate- and differentiation-related target genes, by introducing or removing a binding site for a cell-reprogramming transcription factor (e.g., adding a site for an activating TF or deleting a site that binds a repressor, activating by de-repression, or adding a site for a repressive TF to down-regulate expression). Installation of sequence motifs can be used to drive programmable cellular differentiation from one cell type to another target cell type.

SHAPE to program cellular differentiation (from iPSCs to specific cell types, or specific cell types into another) through the modification of TF gene expression that is involved in cell identity³³. The introduction of sequence motifs to modify (e.g., increase or decrease) the expression of a target gene product (e.g., RNA, protein) can be performed with a genetic modifier, where the resulting change in transcription of a single target gene or multiple target genes leads to differentiation of a cell type to another target cell type. Examples of cell-reprogramming transcription factors are listed in Table 7 below.

TABLE 7
Examples of cell-reprogramming transcription factors

| | Transcription |
|---|---|
| Cell type | factor |
| Embryonic | SALL4 |
| stem cells | OTX2 |
| | ZIC3 |
| | NANOG |
| | ZSCAN10 |
| | POU5F1 |
| | MYCN |
| | NR6A1 |
| | ZIC2 |
| | SOX2 |
| | MYC |
| | KLF4 |
| Neural | OTX2 |
| precursor cells | SALL4 |
| | SIX3 |
| | LHX2 |
| | SP8 |
| | SOX11 |
| | TCF3 |
| | PAX6 |
| | ZIC2 |
| | NR6A1 |
| | FOXG1 |
| | SOX2 |
| | ZIC1 |
| | REST |
| | POU3F2 |
| | HES1 |
| | RFX4 |
| | NEUROG2 |
| | ASCL1 |
| | PLAGL1 |
| | MYC |
| | KLF4 |
| Cardiomyocytes | ANKRD1 |
| | NKX2-5 |
| | E2F8 |
| | TBX5 |
| | MEF2A |
| | ZBF193 |
| | MSX2 |
| | SOX11 |
| | GATA4 |
| | LRRFIP1 |
| | HAND2 |
| Hepatocytes | NR1H4 |
| | NR1I2 |
| | HNF4A |
| | NR5A2 |
| | HNF4G |
| | ATF5 |
| | NR1I3 |
| | HHEX |
| | PROX1 |
| | FOXA3 |
| | CEBPA |
| | GATA4 |
| Motor neurons | MNX1 |
| | ESRRG |
| | ISL2 |
| | HOXC6 |
| | CREM |
| | NHLH1 |
| | ZNF92 |
| | GZF1 |
| | GLIS3 |
| | ISL1 |
| | POU3F2 |
| | MYT1L |
| | ASCL1 |
| | NEUROG2 |
| | LHX3 |
| Pancreatic islet | RFX6 |
| cells | INSM1 |
| | PAX6 |
| | ISL1 |
| | NEUROD1 |
| | GLIS3 |
| | NR5A2 |
| | ZNF165 |
| | ARX |
| | MNX1 |
| | MAFB |
| | PDX1 |
| | PAX4 |
| | NEUROG3 |
| Melanocytes | PAX3 |
| | ALX1 |
| | TFAP2A |
| | MITF |
| | E2F7 |
| | SNAI2 |
| | LZTS1 |
| | ZFY |
| | TCFL5 |
| | PKNOX2 |
| | SOX10 |
| Retinal pigment | OTX2 |
| epithelial | SIX3 |
| | LHX2 |
| | PAX6 |
| | FOXD1 |
| | MITF |
| | C11orf9 |
| | ZNF92 |
| | GLIS3 |
| | SOX9 |
| | NRL |
| | CRX |
| | RAX |
| | MYC |
| | KLF4 |

### Methods for Heritable Target Gene Repression

Methlyation of CpG dinucleotides at promoters or enhancers are known for transcriptional silencing³⁴. This DNA modification is maintained during replication by endogenous DNMT1. Previous studies of introducing methylated DNA at the promoter of reporter plasmids showed the repression of reporter genes³⁵⁻³⁷. SHAPE can be used for the installation of methylated CpGs at the regulatory elements (promoters or enhancers) for heritable target gene repression. To achieve CpG methylation at the promoters or enhancers of target genes, first, we will identify genes that are highly expressed in target cells. Second, we will methylate CpGs for insertion motifs in vitro using methylases and SAM. The methylated CpGs can be inserted into the target regions via genetic modifiers. The in vivo methylation status can be determined via bisulfite genomic sequencing of target regions, and the repression effect of target genes can be validated by RT-qPCR.

non-coding RNA is a common strategy to perform target gene repression, where an RNA sequence with complementarity to a target messenger RNA (mRNA) hybridizes to its target and either accelerates degradation of the mRNA or prevents translation of the mRNA, ultimately reducing target gene protein production. Here we describe a strategy within SHAPE to install sequence motifs that induce targeting of endogenous non-coding RNAs (e.g., miRNAs, siRNAs, lncRNAs) for heritable target gene repression via RNA interference. Following the identification of endogenously expressed endogenous non-coding RNAs (e.g., miRNAs, siRNAs, lncRNAs) in a cell type or cell types of interest, the specific binding sites of these non-coding RNAs can be introduced into the target gene transcript at either the 5′ or 3′ UTR or in intronic regions via genetic modifiers with the ability to introduce insertion edits (e.g., programmable nucleases+ssODNs/dsODNs, prime editors). This method would be validated by RT-qPCR of the target gene transcript to assess RNA knockdown or protein-based assays (e.g., western blot, ELISA) to assess downstream protein knockdown to see if target gene repression is achieved.

SHAPE to Increase or Decrease Transcript Stability, and/or Transcription, and/or Protein Output

The activation of target gene protein output can also be performed through the introduction of sequence motifs in the untranslated region to increase transcript stability. Following the identification or generation of sequence motifs that have the potential to promote RNA stability, genetic modifiers with the ability to introduce insertion edits (e.g., programmable nucleases+ssODNs/dsODNs, prime editors) can be used to target the 5′ and/or 3′ untranslated regions (UTRs) of target genes. The introduction of these RNA-stabilizing sequence motifs can be inserted at all targetable positions of the UTRs to find the optimal positioning of the editing event. The stabilization RNA can be read out through RT-qPCR to quantitate the increase in target RNA abundance that should occur through a decreased rate of RNA degradation.

### Multiplex SHAPE

Multiplex genome editing with programmable nucleases, base editors, and prime editors can be utilized to induce more robust activation for a single target gene. The potential to modify more than one genomic location within regulatory elements of a single target gene may enable additive or synergistic effects for target gene activation. Previous studies showed multiplexed genome engineering of up to 25 human endogenous targets, suggesting up to 25 sites can be simultaneously edited for robust activation of target genes (McCarty, N. S., Graham, A. E., Studená, L. et al. Nat Commun 11, 1281 (2020), Campa, C. C., Weisbach, N. R., Santinha, A. J. et al. Nat Methods 16, 887-893 (2019)). Multiplex editing can be combinations of different strategies, including: 1) introduction of a de novo transcription factor binding site 2) modification of an endogenous transcription factor site to either increase or decrease its regulatory potential 3) modification of endogenous spacing of transcription factor binding sites to increase or decrease regulatory potential. Multiplex editing can be performed within a single regulatory element (e.g., promoter, enhancer), or across multiple regulatory elements.

Multiplex genome editing with programmable nucleases, base editors, and prime editors can also be utilized to induce multi-gene activation. Previous studies showed multiplexed genome engineering of up to 25 human endogenous targets, suggesting up to 25 sites can be simultaneously edited for muti-gene activation (McCarty, N. S., Graham, A. E., Studená, L. et al. Nat Commun 11, 1281 (2020), Campa, C. C., Weisbach, N. R., Santinha, A. J. et al. Nat Methods 16, 887-893 (2019)). The potential to modify more than one genomic location may enable the ability to activate multiple genes in parallel with SHAPE. Multiplex editing can be combinations of different strategies, including: 1) introduction of a de novo transcription factor binding site 2) modification of an endogenous transcription factor site to either increase or decrease its regulatory potential 3) modification of endogenous spacing of transcription factor binding sites to increase or decrease regulatory potential. Multiplex editing can be performed within a single regulatory element (e.g., promoter, enhancer), or across multiple regulatory elements.

### EXAMPLES

The invention is further described in the following examples, which do not limit the scope of the invention described in the claims. The examples described herein show gene expression activation by the SHAPE platform using both dual adenine and cytosine base editors and prime editors to install de novo transcription factor binding sites in HEK293T cells. A variety of transcription factor binding motifs were inserted into endogenous genomic contexts, and gene expression changes were measured at the RNA level. Our findings broaden the capabilities for durable and heritable gene activation by precision genome engineering.

Methods and Materials

The Methods and Materials described herein were used in the Examples provided herein.

Molecular Cloning

All base editor (BE) and prime editor (PE) constructs were cloned into a mammalian expression plasmid backbone under the control of a pCMV promoter (AgeI and NotI restriction digest of parental plasmid Addgene No. 112101). Gibson fragments with matching overlaps were PCR-amplified using Phusion High-fidelity polymerase (NEB). Fragments were gel-purified and assembled for 1 hour at 50° C. and transformed into chemically competent E. coli (XL1-Blue, Agilent). All guide RNA (gRNA) constructs were cloned into a BsmBI-digested pUC19-based entry vector (BPK1520, Addgene No. 65777) with a U6 promoter driving gRNA expression. We designed the pegRNAs following the previously described default design rules for designing pegRNAs and ngRNAs (Anzalone et al, Nature 2019, 576, pages 149-157). PegRNAs were cloned into the Bsal-digested pU6-pegRNA-GG-acceptor entry vector (Addgene No. 132777) and ngRNAs were cloned into the BsmBI-digested entry vector BPK1520 that is mentioned above. Oligos containing the spacer, the 5′phosphorylated pegRNA scaffold, and the 3′ extension sequences were annealed to form dsDNA fragments with compatible overhangs and ligated using T4 ligase (NEB). All plasmids used for transfection experiments were prepared using Qiagen Midi or Maxi Plus kits.

Guide RNAs Used in Nuclease and Base Editor Experiments

All gRNAs for base editors were of the form 5′-NNNNNNNNNNNNNNNNNNNNCGTTTTAGAGCTAGAAATAGCAAGTTAAAATAAGG CTAGTCCGTTATCAACTTGAAAAAGTGGCACCGAGTCGGTGCTTTTTTT-3′. (SEQ ID NO: 4)

TABLE A
Shown below are the spacer regions
(NNNNNNNNNNNNNNNNNNNN in
SEQ ID NO: 4) for these gRNAs
(all written 5′ to 3′).

| Target | | SEQ | | SEQ |
|---|---|---|---|---|
| gene/ | Spacer | ID | Genomic target | ID |
| site | sequence | NO: | sequence | NO: |
| RCC1 | AACCACACAACGCTA | 6 | AACCACACAACGCTAT | 6 |
| promoter | TGACA | | GACA | |
| SAE1 | TGCCACATAAGTGAC | 7 | TGCCACATAAGTGACC | 7 |
| promoter | CACGT | | ACGT | |
| COMT | TACCAGCTCTGGGAG | 8 | TACCAGCTCTGGGAGA | 8 |
| promoter | ACCAC | | CCAC | |
| RARA | TTCCAGCTGGTCCCTA | 9 | TTCCAGCTGGTCCCTAG | 9 |
| promoter | GAAG | | AAG | |
| IVD | GACCAATTTACTGGG | 10 | GACCAATTTACTGGGTA | 10 |
| promoter | TACGT | | CGT | |

Prime Editing Guide RNAs (pegRNAs)

All pegRNAs for prime editors were of the form 5′-NNNNNNNNNNNNNNNNNNNNGTTTTAGAGCTAGAAATAGCAAGTTAAAATAAGGC TAGTCCGTTATCAACTTGAAAAAGTGGCACCGAGTCGGTGC TTTTTTT-3′. (SEQ ID NO: 11)

TABLE B
Shown below are the spacer and 3′ extension sequences
for these pegRNAs (all written 5′ to 3′).

| | | | SEQ | | SEQ |
|---|---|---|---|---|---|
| | | | ID | | ID |
| Target_ | Peg | Spacer_ | NO: | Extension_ | NO: |
| name | RNA | sequence | (#) | sequence | (#) |
| MYOD1 | A | GATAAATAGCCC | 441 | AGGGCTTCCTCACCCCTAGCTTC | 12 |
| | | AGGGCGCC | | TCGCCAGGCgggggccaatcagcGCCC | |
| | | | | TGGGCTATTT | |
| MYOD1 | A | GATAAATAGCCC | 442 | AGGGCTTCCTCACCCCTAGCTTC | 13 |
| | | AGGGCGCC | | TCGCCAGGCgggggccaatcagcgggcca | |
| | | | | atcagcGCCCTGGGCTATTT | |
| MYOD1 | A | GATAAATAGCCC | 443 | AGGGCTTCCTCACCCCTAGCTTC | 14 |
| | | AGGGCGCC | | TCGCCAGGCggggccccgcccccGCCC | |
| | | | | TGGGCTATTT | |
| MYOD1 | A | GATAAATAGCCC | 444 | AGGGCTTCCTCACCCCTAGCTTC | 15 |
| | | AGGGCGCC | | TCGCCAGGCggggccccgcccccggcccc | |
| | | | | gcccccGCCCTGGGCTATTT | |
| MYOD1 | A | GATAAATAGCCC | 445 | AGGGCTTCCTCACCCCTAGCTTC | 16 |
| | | AGGGCGCC | | TCGCCAGGCggcacttccgggttGCCCT | |
| | | | | GGGCTATTT | |
| MYOD1 | A | GATAAATAGCCC | 446 | AGGGCTTCCTCACCCCTAGCTTC | 17 |
| | | AGGGCGCC | | TCGCCAGGCggcacttccgggttcacttccg | |
| | | | | ggttGCCCTGGGCTATTT | |
| MYOD1 | A | GATAAATAGCCC | 447 | AGGGCTTCCTCACCCCTAGCTTC | 18 |
| | | AGGGCGCC | | TCGCCAGGCggccttatctgGCCCTGG | |
| | | | | GCTATTT | |
| MYOD1 | A | GATAAATAGCCC | 448 | AGGGCTTCCTCACCCCTAGCTTC | 19 |
| | | AGGGCGCC | | TCGCCAGGCggccttatctgccttatctgGC | |
| | | | | CCTGGGCTATTT | |
| MYOD1 | A | GATAAATAGCCC | 449 | AGGGCTTCCTCACCCCTAGCTTC | 20 |
| | | AGGGCGCC | | TCGCCAGGCggccttatctgccttatctgcctt | |
| | | | | atctgGCCCTGGGCTATTT | |
| MYOD1 | A | GATAAATAGCCC | 450 | AGGGCTTCCTCACCCCTAGCTTC | 21 |
| | | AGGGCGCC | | TCGCCAGGCggttccttccttccGCCCT | |
| | | | | GGGCTATTT | |
| MYOD1 | A | GATAAATAGCCC | 451 | AGGGCTTCCTCACCCCTAGCTTCTCG | 22 |
| | | AGGGCGCC | | CCAGGCggttccttccttccttccttccttc | |
| | | | | cGCCCTGGGCTATTT | |
| MYOD1 | B | CCTCCCTCCCTGC | 452 | GCCCCTCCACACTGGCGTGCGC | 23 |
| | | CCGGTAG | | GCTAGGTCCCCTAgggggccaatcagc | |
| | | | | CCGGGCAGGGAGGG | |
| MYOD1 | B | CCTCCCTCCCTGC | 453 | GCCCCTCCACACTGGCGTGCGC | 24 |
| | | CCGGTAG | | GCTAGGTCCCCTAgggggccaatcagc | |
| | | | | gggccaatcagcCCGGGCAGGGAGGG | |
| MYOD1 | B | CCTCCCTCCCTGC | 454 | GCCCCTCCACACTGGCGTGCGC | 25 |
| | | CCGGTAG | | GCTAGGTCCCCTAggggccccgccccc | |
| | | | | CCGGGCAGGGAGGG | |
| MYOD1 | B | CCTCCCTCCCTGC | 455 | GCCCCTCCACACTGGCGTGCGC | 26 |
| | | CCGGTAG | | GCTAGGTCCCCTAggggccccgccccc | |
| | | | | ggccccgcccccCCGGGCAGGGAGG | |
| | | | | G | |
| MYOD1 | B | CCTCCCTCCCTGC | 456 | GCCCCTCCACACTGGCGTGCGC | 27 |
| | | CCGGTAG | | GCTAGGTCCCCTAggcacttccgggttC | |
| | | | | CGGGCAGGGAGGG | |
| MYOD1 | B | CCTCCCTCCCTGC | 457 | GCCCCTCCACACTGGCGTGCGC | 28 |
| | | CCGGTAG | | GCTAGGTCCCCTAggcacttccgggttC | |
| | | | | acttccgggttCCGGGCAGGGAGGG | |
| MYOD1 | B | CCTCCCTCCCTGC | 458 | GCCCCTCCACACTGGCGTGCGC | 29 |
| | | CCGGTAG | | GCTAGGTCCCCTAggccttatctgCCG | |
| | | | | GGCAGGGAGGG | |
| MYOD1 | B | CCTCCCTCCCTGC | 459 | GCCCCTCCACACTGGCGTGCGC | 30 |
| | | CCGGTAG | | GCTAGGTCCCCTAggccttatctgccttat | |
| | | | | ctgCCGGGCAGGGAGGG | |
| MYOD1 | B | CCTCCCTCCCTGC | 460 | GCCCCTCCACACTGGCGTGCGC | 31 |
| | | CCGGTAG | | GCTAGGTCCCCTAggccttatctgccttat | |
| | | | | ctgccttatctgCCGGGCAGGGAGGG | |
| MYOD1 | B | CCTCCCTCCCTGC | 461 | GCCCCTCCACACTGGCGTGCGC | 32 |
| | | CCGGTAG | | GCTAGGTCCCCTAggttccttccttccC | |
| | | | | CGGGCAGGGAGGG | |
| MYOD1 | B | CCTCCCTCCCTGC | 462 | GCCCCTCCACACTGGCGTGCGC | 33 |
| | | CCGGTAG | | GCTAGGTCCCCTAggttccttccttccttc | |
| | | | | cttccttccCCGGGCAGGGAGGG | |
| MYOD1 | C | GAGGTTTGGAAA | 463 | GGCAGCCCTAGGCGGCTGCACT | 34 |
| | | GGGCGTGC | | TGGCTCTCCGGCAgggggccaatcagc | |
| | | | | CGCCCTTTCCAAAC | |
| MYOD1 | C | GAGGTTTGGAAA | 464 | GGCAGCCCTAGGCGGCTGCACT | 35 |
| | | GGGCGTGC | | TGGCTCTCCGGCAgggggccaatcagc | |
| | | | | gggccaatcagcCGCCCTTTCCAAAC | |
| MYOD1 | C | GAGGTTTGGAAA | 465 | GGCAGCCCTAGGCGGCTGCACT | 36 |
| | | GGGCGTGC | | TGGCTCTCCGGCAggggccccgccccc | |
| | | | | CGCCCTTTCCAAAC | |
| MYOD1 | C | GAGGTTTGGAAA | 466 | GGCAGCCCTAGGCGGCTGCACT | 37 |
| | | GGGCGTGC | | TGGCTCTCCGGCAggggccccgccccc | |
| | | | | ggccccgcccccCGCCCTTTCCAAAC | |
| MYOD1 | C | GAGGTTTGGAAA | 467 | GGCAGCCCTAGGCGGCTGCACT | 38 |
| | | GGGCGTGC | | TGGCTCTCCGGCAggcacttccgggttC | |
| | | | | GCCCTTTCCAAAC | |
| MYOD1 | C | GAGGTTTGGAAA | 468 | GGCAGCCCTAGGCGGCTGCACT | 39 |
| | | GGGCGTGC | | TGGCTCTCCGGCAggcacttccgggttC | |
| | | | | acttccgggttCGCCCTTTCCAAAC | |
| MYOD1 | C | GAGGTTTGGAAA | 469 | GGCAGCCCTAGGCGGCTGCACT | 40 |
| | | GGGCGTGC | | TGGCTCTCCGGCAggccttatctgCGC | |
| | | | | CCTTTCCAAAC | |
| MYOD1 | C | GAGGTTTGGAAA | 470 | GGCAGCCCTAGGCGGCTGCACT | 41 |
| | | GGGCGTGC | | TGGCTCTCCGGCAggccttatctgccttat | |
| | | | | ctgCGCCCTTTCCAAAC | |
| MYOD1 | C | GAGGTTTGGAAA | 471 | GGCAGCCCTAGGCGGCTGCACT | 42 |
| | | GGGCGTGC | | TGGCTCTCCGGCAggccttatctgccttat | |
| | | | | ctgccttatctgCGCCCTTTCCAAAC | |
| MYOD1 | C | GAGGTTTGGAAA | 472 | GGCAGCCCTAGGCGGCTGCACT | 43 |
| | | GGGCGTGC | | TGGCTCTCCGGCAggttccttccttccC | |
| | | | | GCCCTTTCCAAAC | |
| MYOD1 | C | GAGGTTTGGAAA | 473 | GGCAGCCCTAGGCGGCTGCACT | 44 |
| | | GGGCGTGC | | TGGCTCTCCGGCAggttccttccttccttc | |
| | | | | cttccttccCGCCCTTTCCAAAC | |
| HBB | D | GGCTCTTCTGGCA | 474 | GAGGGAGGGCTGAGGGTTTGAA | 45 |
| | | CTGGCTT | | GTCCAACTCCTAAGcacttccgggttca | |
| | | | | cttccgggttCCAGTGCCAGAAGA | |
| IL2RA | E | GGATGAGAGAAG | 475 | ATTGGGCTGGCGTGTTCAGCCA | 46 |
| | | AGAGTGCT | | GGAAACTGCCTAGCcacttccgggttca | |
| | | | | cttccgggttACTCTCTTCTCTCA | |
| HER2 | F | GCCCTCTCTTCGC | 476 | AGGCGTCCCGGCGCTAGGAGGG | 47 |
| | | GCAGGCCT | | ACGCACCCAGGcacttccgggttcacttcc | |
| | | | | gggttCCTGCGCGAAGA | |
| EpCAM | G | GAGCGCACACCT | 477 | TCCCGGGAAGGGGCCGAGAGG | 48 |
| | | GCCCGACC | | CGGGGCCGCCAGGTcacttccgggttc | |
| | | | | acttccgggttCGGGCAGGTGTG | |

# SEQ ID NO:

PE3 nicking guide RNAs (ngRNAs)

All nicking gRNAs for PE3 system were of the form 5′-NNNNNNNNNNNNNNNNNNNNCGTTTTAGAGCTAGAAATAGCAAGTTAAAATAAGG CTAGTCCGTTATCAACTTGAAAAAGTGGCACCGAGTCGGTGCTTTTTTT-3′. (SEQ ID NO: 49)

TABLE C
Shown below are the protospacer regions
for these nicking gRNAs
(NNNNNNNNNNNNNNNNNNNN in SEQ ID NO: 49,
all written 5′ to 3′).

| Target | | | SEQ ID |
|---|---|---|---|
| gene | pegRNA | Protospacer sequence | NO: |
| MYOD1 | A | GAGGCCAATAGGAACACTGC | 50 |
| MYOD1 | A | CTCCTGTCCGGCCTGATTTG | 51 |
| MYOD1 | A | CCACCCCTCCCTCTCCTGTC | 52 |
| MYOD1 | B | CACCCGACTGCTGTATCCGC | 53 |
| MYOD1 | B | GGCCGCAGGGGCCCGCAGAC | 54 |
| MYOD1 | B | CCAATAGGAACACTGCGGGG | 55 |
| MYOD1 | C | CTCCTCTGTCCCCTGATTTG | 56 |
| MYOD1 | C | GCTAGGTCCCCTACCGGGCA | 57 |
| MYOD1 | C | TCCACACTGGCGTGCGCGCT | 58 |
| HBB | D | GTGGAGCCACACCCTAGGGT | 59 |
| IL2RA | E | GTTGATGACAATATAGTTTG | 60 |
| HER2 | F | GCTGCATTTAGGGATTCTCCG | 61 |
| EpCAM | G | GACTCATCAACGAGCACCAG | 62 |

Cell Culture and Transfections

STR-authenticated HEK293T (CRL-3216), K562 (CCL-243), HeLa (CCL-2), and U2OS cells (similar match to HTB-96; gain of #8 allele at the D5S818 locus) were used in this study. HEK293T and HeLa cells were grown in Dulbecco's Modified Eagle Medium (DMEM, Gibco) with 10% heat-inactivated fetal bovine serum (FBS, Gibco) supplemented with 1% penicillin-streptomycin (Gibco) antibiotic mix. K562 cells were grown in Roswell Park Memorial Institute (RPMI) 1640 Medium (Gibco) with 10% FBS supplemented with 1% Pen-Strep and 1% GlutaMAX (Gibco). U2OS cells were grown in DMEM with 10% FBS supplemented with 1% Pen-Strep and 1% GlutaMAX. Cells were grown at 37° C. in 5% CO2 incubators and periodically passaged upon reaching around 80% confluency. Cell culture media supernatant was tested for mycoplasma contamination using the MycoAlert mycoplasma detection kit (Lonza) and all tests were negative throughout the experiments.

Transfections

HEK293T cells were seeded at 1.25×10⁴ cells per well into 96-well flat bottom cell culture plates (Corning) for DNA on-target experiments or at 6.25×10⁴ cells per well into 24-well cell culture plates (Corning). 24 hours post-seeding, cells were transfected with 30 ng of control or base/prime editor plasmid and 10 ng of gRNA plasmid (and 3.3 ng nicking gRNA plasmid for PE3) using 0.3 μL of TransIT-X2 (Minis) lipofection reagent for experiments in 96-well plates, 150 ng control or base editor plasmid and 50 ng gRNA, or 375 ng dCas9-VPR and 125 ng gRNA, and 3 μL TransIT-X2 for experiments in 24-well plates. K562 cells were electroporated using the SF Cell Line Nucleofector X Kit (Lonza) or Kit V (Lonza), according to the manufacturer's protocol with 2×10⁵ cells per nucleofection and 800 ng control or base/prime editor plasmid, 200 ng gRNA or pegRNA plasmid, and 83 ng nicking gRNA plasmid (for PE3) or with 1×10⁶ cells per nucleofection and 3840 ng control or prime editor plasmid, 960 ng pegRNA plasmid, and 398.4 ng of nicking gRNA plasmid (for PE3), and 3750 ng dCas9-VPR plasmid and 1250 ng of gRNA. When GFP plasmid was co-transfected, half amount of gRNAs or nicking gRNAs was used. U2OS cells were electroporated using the SE Cell Line Nucleofector X Kit (Lonza) with 2×10⁵ cells and 800 ng control or base/prime editor plasmid, 200 ng gRNA or pegRNA, and 83 ng nicking gRNA (for PE3). HeLa cells were electroporated using the SE Cell Line 4D-Nucleofector X Kit (Lonza) with 5×10⁵ cells and 800 ng control or base/prime editor, 200 ng gRNA or pegRNA, and 83 ng nicking gRNA (for PE3). 72 hours post-transfection, cells were lysed for extraction of genomic DNA (gDNA).

DNA and RNA Extraction

For DNA on-target experiments in 96-well plates, 72 h post-transfection, cells were washed with PBS, lysed with freshly prepared 43.54, DNA lysis buffer (50 mM Tris HCl pH 8.0, 100 mM NaCl, 5 mM EDTA, 0.05% SDS), 5.25 μL Proteinase K (NEB), and 1.25 μL 1M DTT (Sigma). For DNA off-target experiments in 24-well plates, cells were lysed in 174 DNA lysis buffer, 21 μL Proteinase K, and 5 μL 1M DTT. For RNA off-target experiments, GFP sorted cells were split 20% for DNA and 80% for RNA extraction. Cells were centrifuged (200 g, 8 min) and lysed as above for DNA or with 350 μL RNA lysis buffer LBP (Macherey-Nagel) for RNA. DNA lysates were incubated at 55° C. on a plate shaker overnight, then gDNA was extracted with 2× paramagnetic beads (as previously described), washed 3 times with 70% EtOH, and eluted in 30-80 μL 0.1×EB buffer (Qiagen). RNA lysates were extracted with the NucleoSpin RNA Plus kit (Macherey-Nagel) following the manufacturer's instructions.

Targeted Amplicon Sequencing

DNA targeted amplicon sequencing was performed as previously described (Grunewald et al, Nature 2019, 569, pages 433-437). Briefly, extracted gDNA was quantified using the Qubit dsDNA HS Assay Kit (Thermo Fisher). Amplicons were constructed in 2 PCR steps. In the first PCR, regions of interest (170-250 bp) were amplified from 5-20 ng of gDNA with primers containing Illumina forward and reverse adapters on both ends. PCR products were quantified on a Synergy HT microplate reader (BioTek) at 485/528 nm using a Quantifluor dsDNA quantification system (Promega), pooled and cleaned with 0.7× paramagnetic beads, as previously described. In a second PCR step (barcoding), unique pairs of Illumina-compatible indexes (equivalent to TruSeq CD indexes, formerly known as TruSeq HT) were added to the amplicons. The amplified products were cleaned up with 0.7× paramagnetic beads, quantified with the Quantifluor or Qubit systems, and pooled before sequencing. The final library was sequenced on an Illumina MiSeq machine using the Miseq Reagent Kit v2 (300 cycles, 2×150 bp, paired-end). Demultiplexed FASTQ files were downloaded from BaseSpace (Illumina).

Targeted Amplicon Sequencing Analysis

Amplicon sequencing data were analyzed with CRISPResso2 2.0.3016 that was run in base editor output mode. Allele frequency tables (CRISPResso output) display an editing window that includes the edited As or Cs (CCA motif in positions 3-4-5 with 1 being the most PAM-distal base).

Flow Cytometry

Cells were washed with cell staining buffer (Biolegends) after 72 hours post-transfection and incubated with PE conjugated IL2RA (Biolegends), HER2 (Biolegends) or EpCAM antibody (Biolegends) for 15 minutes, followed by twice wash with cell staining buffer. All PE positive cells were sorted and sorted cells were measured by a LSR Fortessa X-20 flow cytometer (BD) to test durability of target protein expression. Cells transfected with GFP plasmid were resuspended in cell staining buffer after 72 hours post-transfection and top 50% GFP positive cells were sorted.

Analysis of Potential SPACE-Encodable Transcription Factor Binding Sites

Annotated transcription start sites from hg38 refseq genes were obtained and filtered to exclude micro-RNAs, small NF90 associated RNAs (SNARs), long non-coding RNAs, small nucleolar RNAs, and anti-sense transcripts. These RNAs were filtered in part due to redundant annotations at the same transcription start sites (TSS) and to focus on protein-coding genes. Next, the remaining TSSes were padded to include the region −500 bp to 0 bp relative to the start site. From this 500 bp per-gene window, we found all matches of the “NNCCA GG” (SEQ ID NO:5) motif on either strand that contain a preferential dual-editing window for SPACE and a canonical SpCas9-PAM (NGG). In total, this defined 53,750 protospacers (45,383 unique). To assess the potential for TF motif creation, we used the reference sequence to create “SPACE-edited” sequences for each of the protospacers by modifying the CCA to TTG. Using a set of 386 transcription motifs from the JASPAR2016 motif list20 we determined which motifs could be created with the SPACE modification for each transcription factor. Motif matching was performed using the motifmatchr package using default parameters as part of the chromVAR suite of tools21. Created motifs were those that did not occur in the reference sequence but were matches in the SPACE-edited sequence.

Measurement of Target Gene Expression for Inserting TF Motis by PE3

HEK293T were transfected with PE3 (60 ng), pegRNA(20 ng) and nicking gRNA (6.64 ng). HEK293T was transfected using lipofection. 24 hours prior to transfection, HEK293T cells (625000) were seeded in 24-well plates and then transfected with the plasmids using 3 μl of Transit X2(Mirus Bio, cat #MIR6003) for HEK293T cells. For target gene expression analysis, total RNA was extracted from the cells 72 hours post-transfection using the NucleoSpin RNA Plus Kit (Clontech, cat #740984.250) and 250 ng of purified RNA was used for cDNA synthesis using a High Capacity RNA-to-cDNA kit (ThermoFisher, cat #4387406). 3 μl of 1:20 diluted cDNA was amplified by quantitative PCR (qPCR) using Fast SYBR Green Master Mix (ThermoFisher, cat #4385612) with the primers listed elsewhere in this application. qPCR reactions were performed on a LightCycler 480 (Roche) with the following program: initial denaturation at 95° C. for 20 seconds (s) followed by 45 cycles of 95° C. for 3 s and 60° C. for 30 s. Ct values greater than 35 were considered as 35, because Ct values fluctuate for transcripts expressed at very low levels. Gene expression levels were normalized to HPRT1 and calculated relative to that of the negative controls (PE3 with pegRNA cassette).

### Example 1: Introducing De Novo Transcription Factor Binding Sites for Gene Activation with Dual Adenine and Cytosine Base Editors

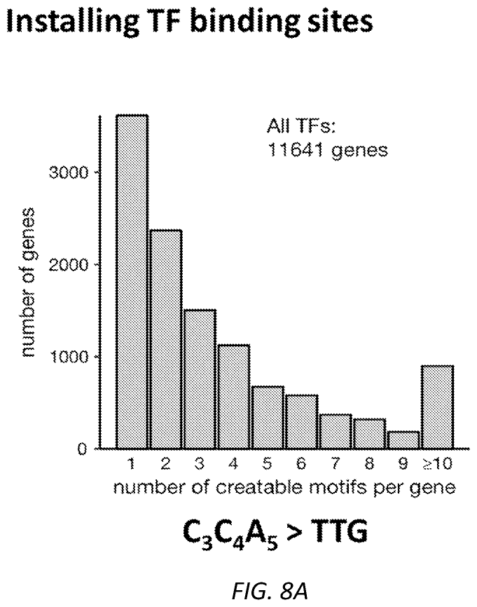

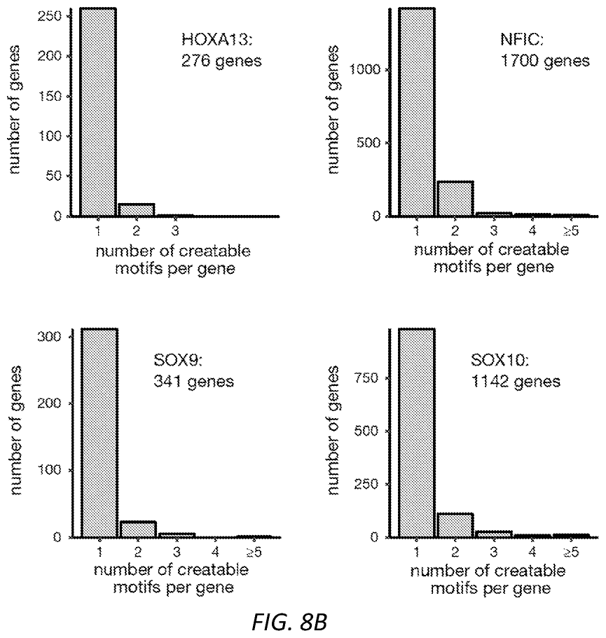

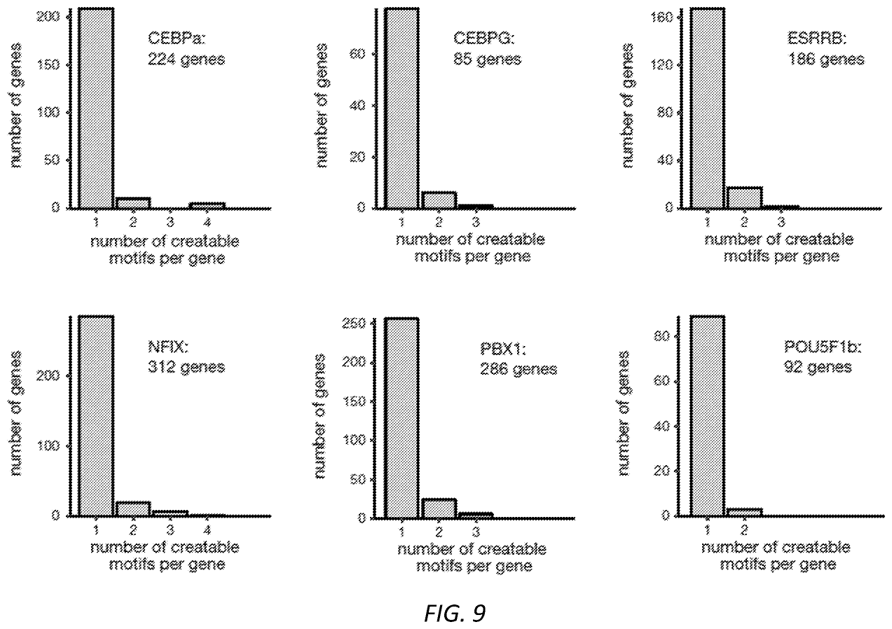

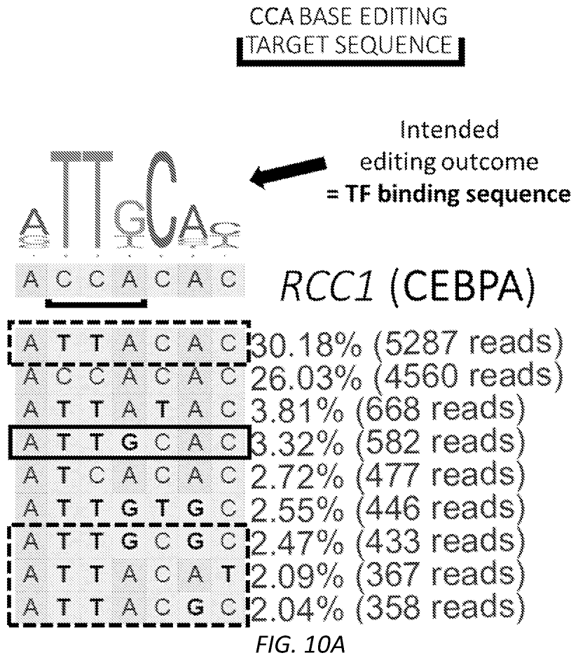

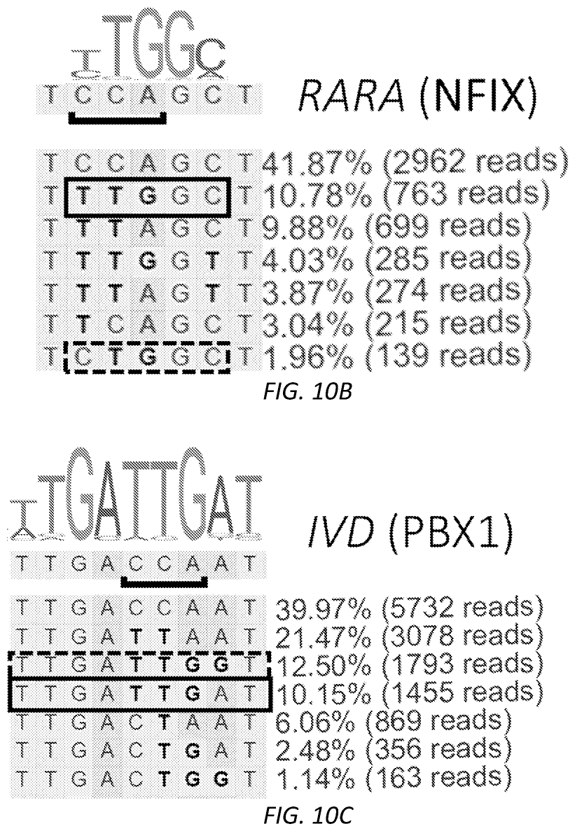

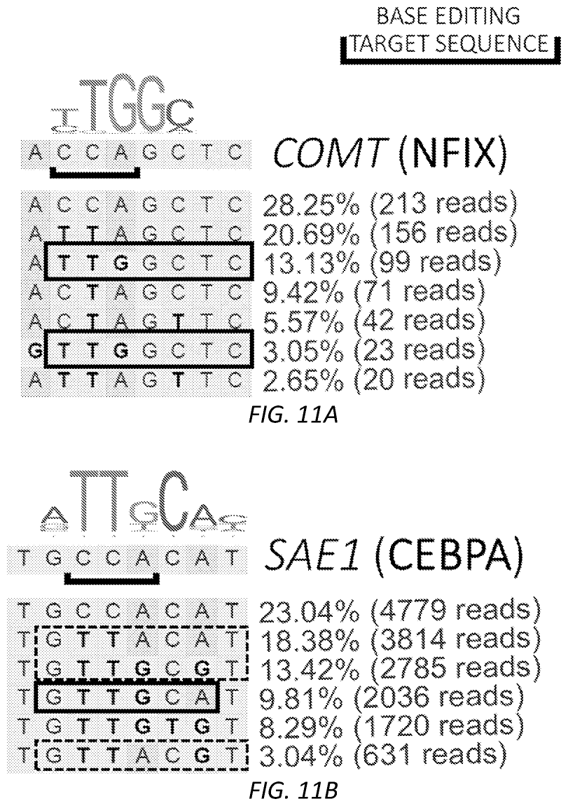

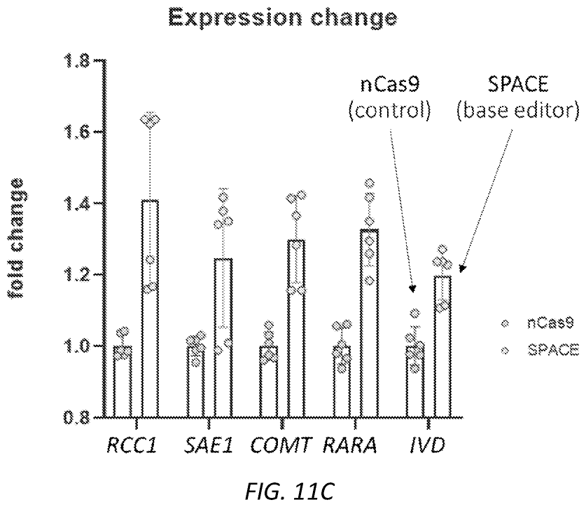

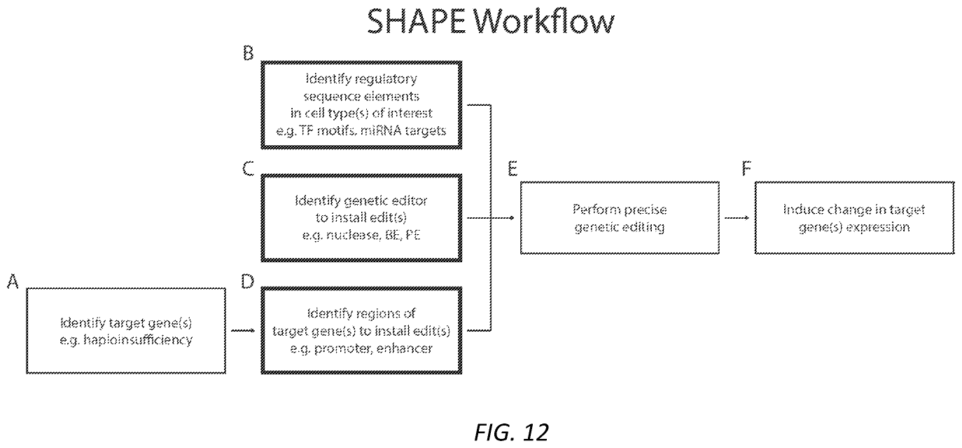

First, a bioinformatic analysis was performed to generate candidate genes with endogenous promoter sequences that can get converted into TF binding sites using SPACE. Potential target sites had to lie within −500 to 0 bp upstream of the TSS, have a C3C4A5 motif, with respect to the protospacer (1 being the most PAM-distal position). Only sites with a canonical NGG-PAM were considered. The number of genes with one or more creatable TF binding sites are shown in FIG. 8. Subsequently, five genes were selected for a BE-SHAPE proof-of-concept pilot experiment. HEK293T cells were transfected in duplicate with plasmids co-expressing either a nCas9 negative control or the dual-deaminase base editor SPACE as well as a gRNA targeting a genomic site in the promoter region of one of these 5 genes of interest (Table A). 48 hours after transfection, cells were trypsinized and split into a 96-well plate for DNA extraction and maintained in a 24-well plate for RNA extraction. Genomic DNA and RNA was harvested 72 hours post-transfection. DNA was used to create NGS-compatible libraries that were run on an Illumina MiSeq and analyzed using CRISPResso2 software. Two examples shown in FIGS. 9a and b indicate efficient dual base editing with SPACE at the target sites. RNA of cells from the same experiment was harvested, reverse transcribed using the High Capacity Kit from Applied Biosystems and used in RT-qPCR experiments (triplicate qPCR per condition) to determine the Ct values of nCas9 and SPACE experiments. The fold-change expression changes were calculated and showed an upregulation of expression following SPACE treatment (base editing) in all 5 genes tested (FIG. 9c). These data indicate that BE-SHAPE enabled the successful upregulation of targeted genes of interest.

### Example 2: Activation of MYOD1 Expression Through the Insertion of Transcription Factor Binding Sites Via Prime Editing

We tested the SHAPE system at the MYOD1 locus in HEK293T cells. First, we identified candidate transcription factor motif sequences to insert into varying positions of the MYOD1 locus, ranging from 250 bp upstream and 50 bp downstream of the MYOD1 transcription start site (TSS). We identified ELF, NFY, and SP transcription factors as being actively expressed in HEK293T cells, and additionally included GATA1 and EWS-FLI1 motifs as positive controls where exogenous GATA1 and EWS-FLI1 would be supplied exogenously (Table D).

TABLE D
Transcription factor motifs for transcription
factors that are expressed in target cell
lines HEK293T, U20S, and K562 (ELF, NFY, SP1)
and factors that are not expressed in target
cell lines (EWS-FLU, GATA1).

| Motif name | Motif sequence | SEQ ID NO: |
|---|---|---|
| ELF | AACCCGGAAGTG | 63 |
| NFY | GCTGATTGGCCC | 64 |
| GATA1 | CAGATAAGG | 65 |
| SP1 | GGGGGCGGGGCC | 66 |
| EWS-FLI1 | GGAA | 67 |

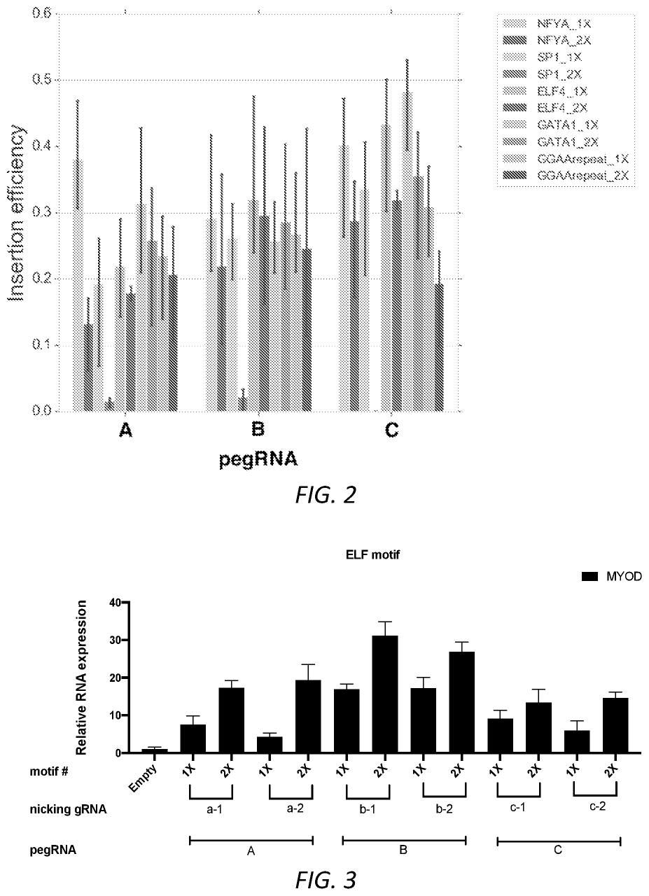

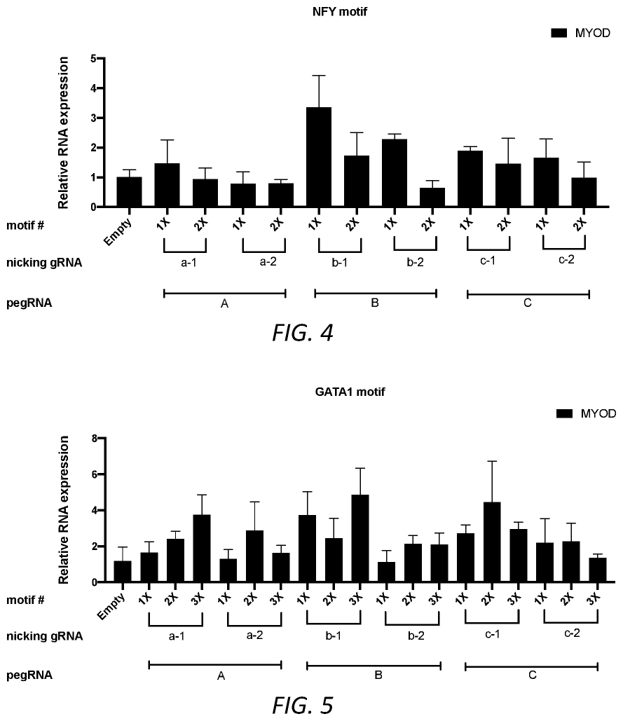

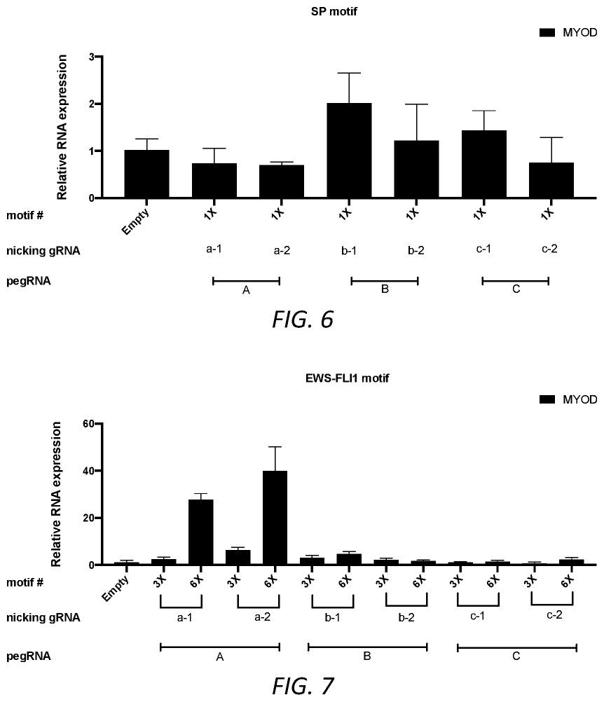

Next, we designed prime editing guide RNAs (pegRNAs) and nicking sgRNAs (ngRNAs) targeting nearby the MYOD1 TSS (Table B and C. We tested prime editing using the PE3 strategy and achieved meaningful insertion editing efficiencies across three different pegRNAs and 10 different motif insertions (FIG. 2). Following the successful insertion of transcription factor motifs at the MYOD1 locus by PE3, we assessed the changes in MYOD1 gene expression by RT-qPCR (see methods and materials). The ELF motif exhibited copy-dependent increases in MYOD1 expression, where ELF 2× yielded greater gene activation compared to ELF 1× across all pegRNA and ngRNA combinations (FIG. 3). The ELF motif insertion was able to promote gene activation that was up to 30-fold over the negative control. The SP, NFY, and GATA1 motif insertions yielded very modest increases in gene expression, with no clear advantages of introducing two copies (2×) of the motif compared to one copy (1×) (FIGS. 4, 5, 6). The EWS-FLI1 experiment was performed by providing an exogenous EWS-FLI1 activator by plasmid, and demonstrated meaningful activation levels for pegRNA A, giving gene activation levels of 30-50 fold greater than the negative control (FIG. 7). Only the EWS-FLI1 6× insertion worked, while the 3× insertion was much weaker in its ability to activate MYOD1 gene expression. These results are consistent with the understanding that EWS-FLI1 typically binds to a minimum repeat sequence of 4×.

### Example 3: Stable and Durable Gene Activation or Protein Expression Via Inserting ELF(2×) Motif at Different Endogenous Human Gene Promoters by Enriching Edited Population with Sorting Cells Based on GFP Plasmid Co-Transfected or Sorting Based on Expression of Cell Surface Marker Target Genes

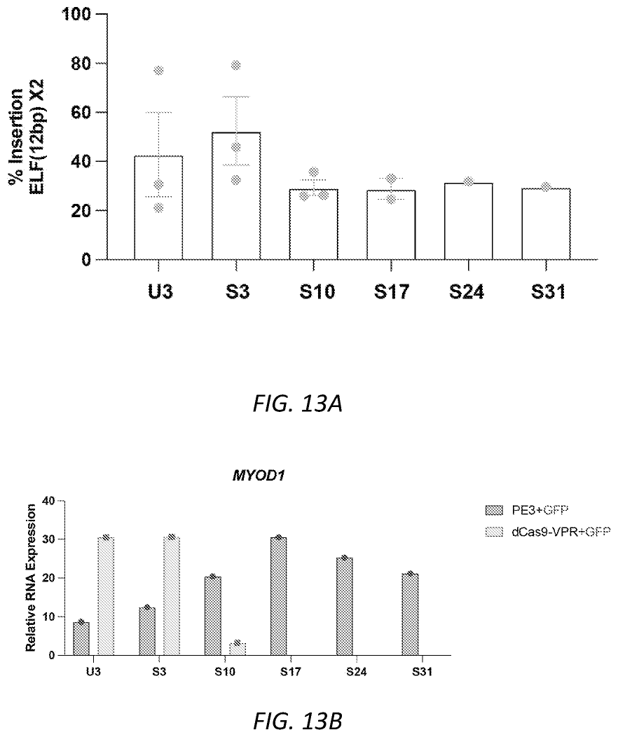

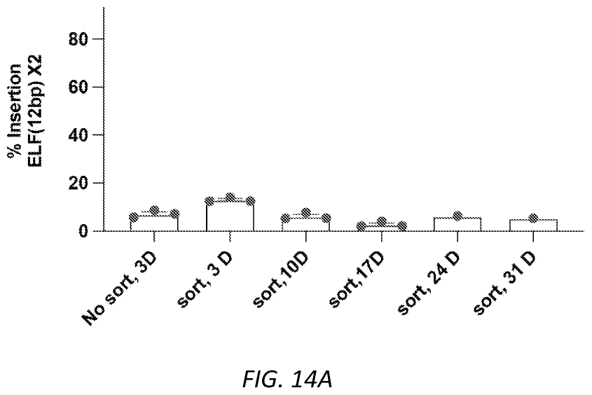

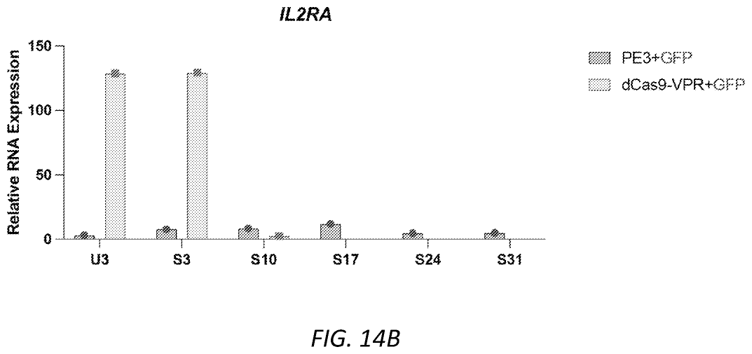

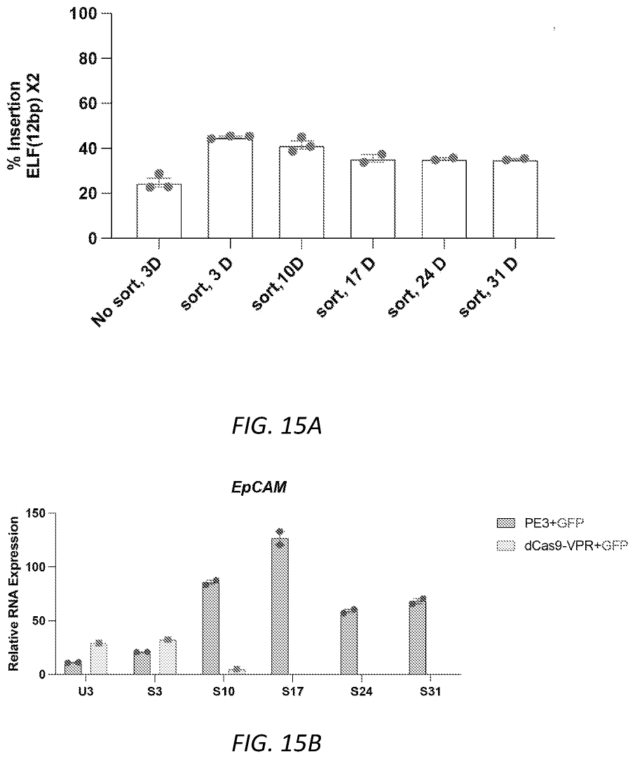

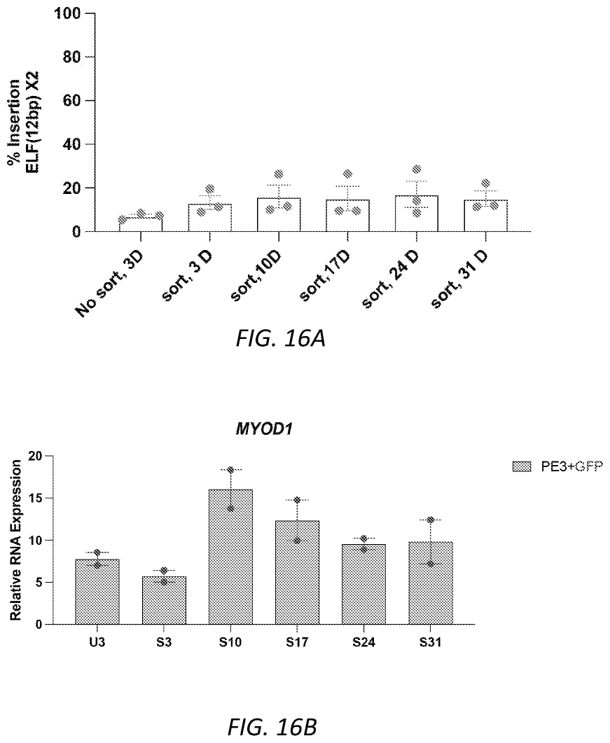

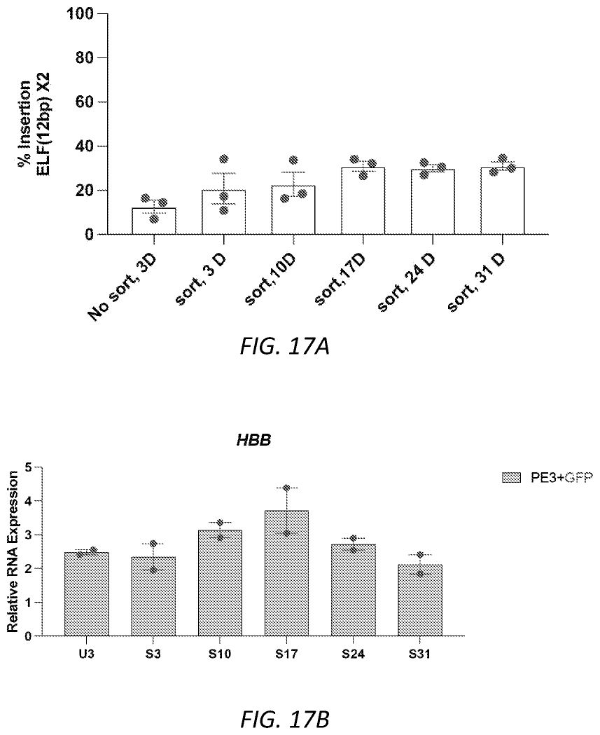

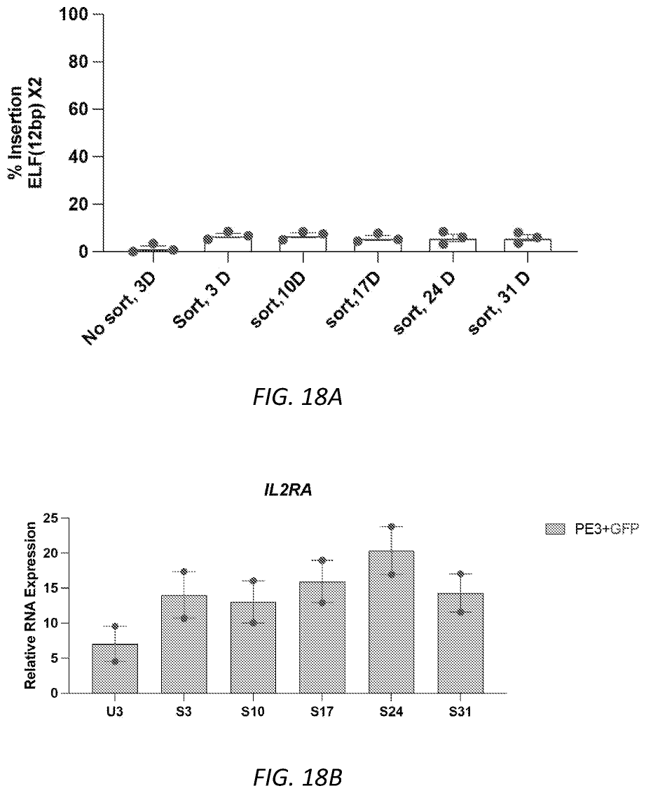

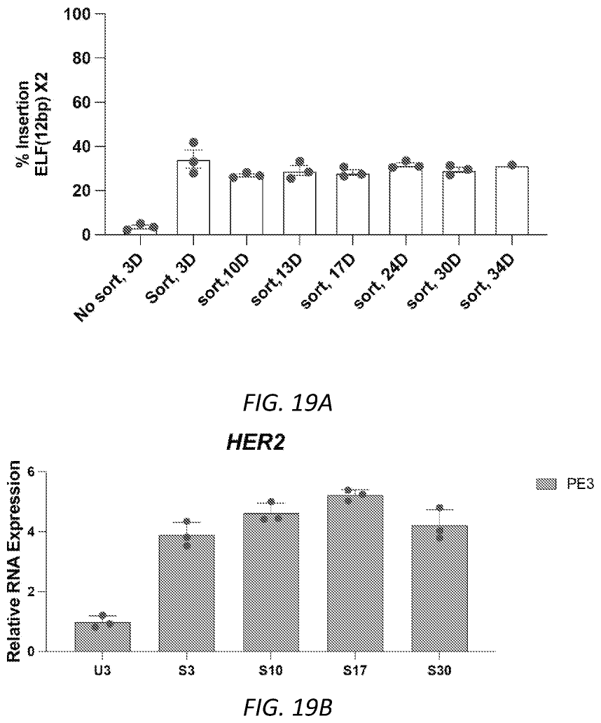

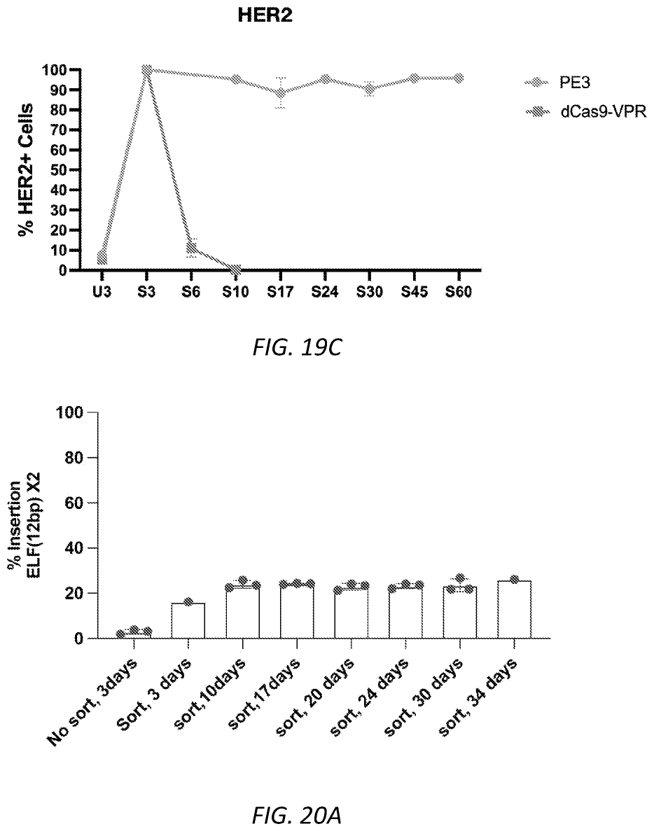

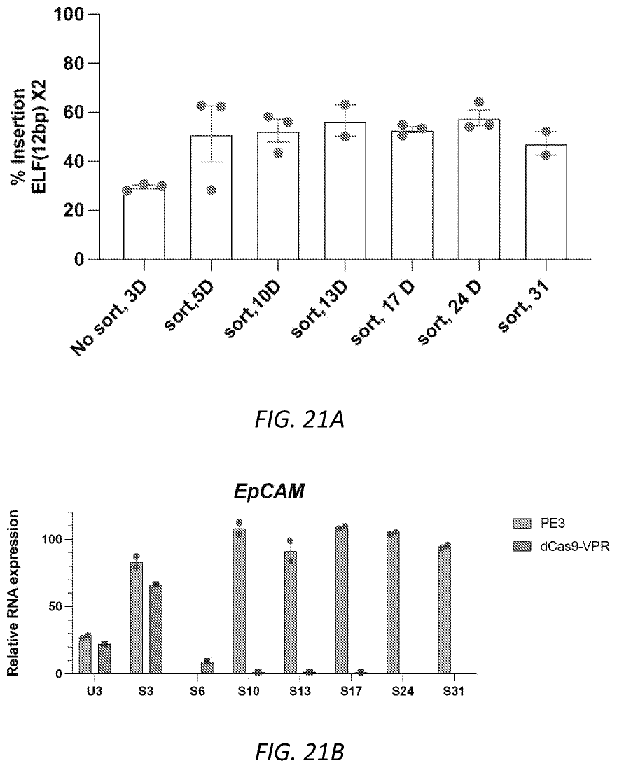

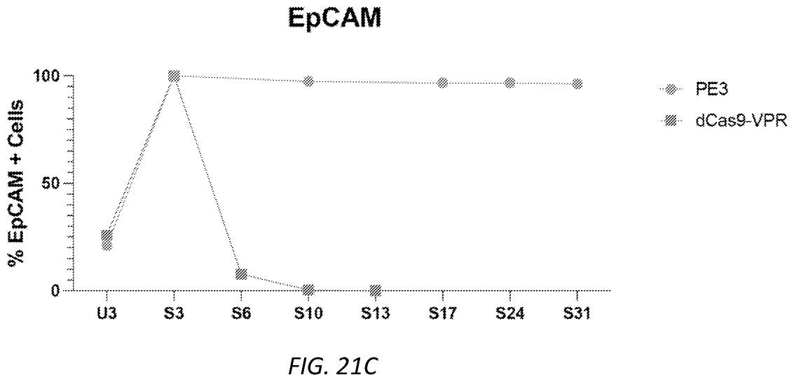

We tested the durability of the SHAPE system at various loci of endogenous promoters of MYOD1, HBB, IL2RA, HER2, and EpCAM in HEK293T and K562. To do so, we first enriched edited cells via sorting cells for GFP signal or target protein expression. To sort cells for GFP signal, cells were co-transfected with a separate GFP plasmid with prime editor complex that was designed to install ELF(2×) motif at the MYOD1, HBB, IL2RA, HER2, or EpCAM. To compare the durability of SHAPE with CRISPRa, cells co-transfected with dCas9-VPR targeting the same target regions and GFP plasmid were also sorted for GFP. We confirmed stable ELF(2×) insertion at the target promoters and stable target mRNA expression over 30 days post-transfection for GFP sorted HEK293T and K562 cells (FIGS. 13-18). However, dCas9-VPR transfected cells showed rapid decrease of target gene expression between 3 and 10 post-transfection (FIGS. 13-15). Next, instead of enriching cells based on GFP protein expression, we used a different enrichment method that sorts cells for target protein expression. With the assumption that inserting ELF2× at the promoters of cell surface marker genes (IL2RA, HER2 and EpCAM) would lead to an increase in their mRNAs followed by protein expression, we sorted cells for cell surface marker proteins. Since this method differentiating cells that have a functional outcome of ELF2× insertion, the insertion efficiency was higher than sorting methods based on GFP signal. We used the same sorting strategy for dCas9-VPR transfected cells that express the same cell surface marker proteins. We observed significantly higher levels of mRNA and protein expression of HER2, IL2RA, and EpCAM for sorted cells compared to the ones from the unsorted populations for both SHAPE and dCas9-VPR approaches. While dCas9-VPR approach showed that the elevated expression of target mRNA and protein expression after sorting dropped drastically between three and 10 days post-transfection, SHAPE via prime editing maintained stable ELF(2×) insertion, mRNA expression, and protein expression over 30 days post-transfection (FIGS. 19-21).

### Example 4: Pooled Screening of Diverse Transcription Factor Cluster Motifs for SHAPE-Mediated Target Protein Expression

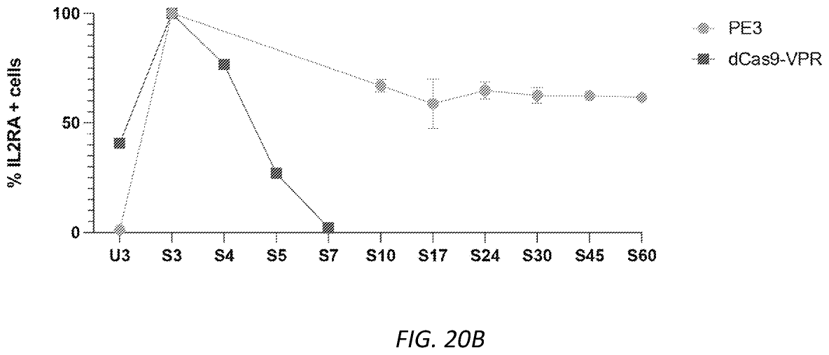

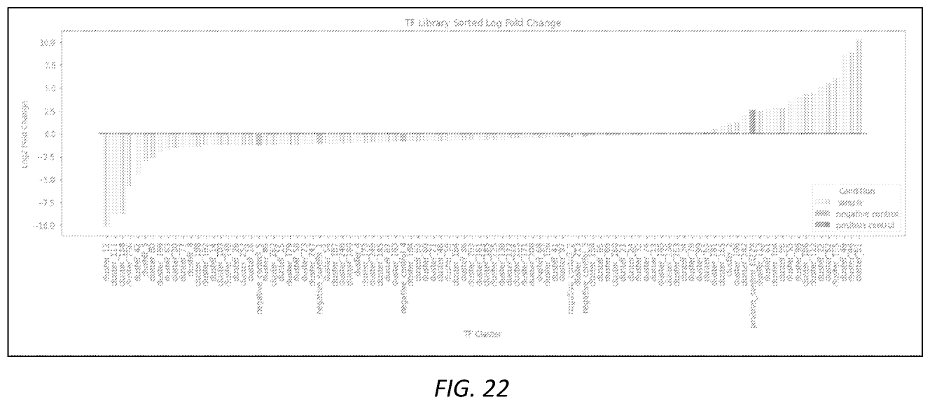

We explored the insertion of diverse transcription factor cluster motifs via prime editing to achieve target protein expression. To do so, we used the IL2RA promoter due to our previous success in activating IL2RA protein expression and the ability to sort for its expression as a cell surface marker (FIG. 20). For the exploration of diverse transcription factor cluster motifs, we used a database of clustered transcription factor motifs³⁸, and chose 94 of these clustered motifs based on transcription factor expression in a variety of cell types (K562, HEK293T, U2OS) (Table E). The chosen cluster motifs were all normalized to 24 bases by extending the motifs through the addition of repeating copies of the motif until the 24 bp length was reached. Additionally, we included a positive control ELF(2×) motif which we had previously shown to activate IL2RA protein expression, along with 5 negative control sequences (24 bp) created with a random DNA generator. All cluster motifs and control sequences were inserted at the same position within the IL2RA promoter using the pegRNA spacer sequence and ngRNA described previously (Table B, C). The pegRNA constructs encoding all cluster motifs and control sequences, in addition to the ngRNA, were pooled and double-electroporated into K562 with the electroporation events separated by 72 hours. Seven days after the second electroporation event, the K562 cells were sorted based on IL2RA protein expression into negative and positive populations. Genomic DNA was harvested from both the IL2RA negative and positive populations and targeted amplicon sequencing was performed at the prime editing insertion location. Each cluster motif insertion was counted within the sequencing reads in both the negative and positive samples, normalized based on total read count in each sample, and an enrichment score was calculated (FIG. 22). We found that the positive control ELF(2×) motif exhibited positive enrichment (FIG. 22, black) and that the 5 negative controls showed very little enrichment (FIG. 22, dark grey). Several of the cluster motifs within the library showed potential to repress and activate target protein expression, shown in the left and right tails of the plot, respectively.

TABLE E
Archetype sequences derived from hierarchal-
clustered transcription factor motifs and
relevant controls for pooled screening of
activating or repressing regulatory elements.

| | | | SEQ |
|---|---|---|---|
| | | | ID |
| Cluster | Name | Motif sequence | NO: |
| 1 | HD/1 | TGATTGCAATCA | 68 |
| 2 | HD/2 | CTAATTAA | 69 |
| 3 | HD/3 | TAATCGGAAAACGATTA | 70 |
| 4 | HD/4 | TTAAGTGC | 71 |
| 5 | HD/5 | AATTAAAAACCAATTA | 72 |
| 6 | HD/6 | AATTGGTGCTAAATG | 73 |
| 7 | HD/7 | TAATTAGCCAATTA | 74 |
| 8 | HD/8 | TAATTAAATTA | 75 |
| 9 | HD/9 | AATTAATTAAT | 76 |
| 10 | HD/10 | GTTAATGATTAAC | 77 |
| 11 | HD/11 | TAATCATCATTA | 78 |
| 12 | HD/12 | TGATTGACAG | 79 |
| 13 | NFY | AGCCAATCAG | 80 |
| 14 | CUX/1 | AAATCGATA | 81 |
| 15 | CUX/3 | ATCGATAACTGATCGAT | 82 |
| 16 | CUX/2 | ATCGATAACCCTATCGAT | 83 |
| 17 | CUX/4 | AATCAATGA | 84 |
| 18 | HD/13 | TTTATG | 85 |
| 19 | HD/14 | TGATTTATG | 86 |
| 20 | HD/15 | TGATGGATG | 87 |
| 21 | HD/16 | TTGATTAAT | 88 |
| 22 | HD/17 | GTCGTAAAA | 89 |
| 23 | CPEB1 | CAATAAAA | 90 |
| 24 | HD/18 | GCAATAAAA | 91 |
| 25 | HD/19 | TAATCCGATTA | 92 |
| 26 | HD/20 | GGATTA | 93 |
| 27 | HD/21 | TCAAGGA | 94 |
| 28 | HD/22 | CCACTTGA | 95 |
| 29 | TCF/LEF | AAGATCAAAG | 96 |
| 30 | NR/1 | GGGTCAAAGGTCA | 97 |
| 31 | NR/2 | AAGTCAAAAGTCA | 98 |
| 32 | NR/3 | TGGGGCAAAGGTCA | 99 |
| 33 | NR/4 | GGGTCATGACCC | 100 |
| 34 | NR/5 | TGACCTTATAAGGTCA | 101 |
| 35 | NR/6 | TGACCTTTAAAGGTC | 102 |
| 36 | NR/7 | GGTCATTCAAGGTCA | 103 |
| 37 | NR/8 | TGACCTTACATAAGGTCA | 104 |
| 38 | NR/9 | AGGTCAAACTGTGACCT | 105 |
| 39 | NR/10 | TGACCTTATTAAGGTCA | 106 |
| 40 | NR/11 | AAGTTCAAGGTCA | 107 |
| 41 | NR/12 | AGGTCATCCAAAGGTCA | 108 |
| 42 | NR/13 | AGGTCAAAAGGTCA | 109 |
| 43 | NR/14 | GTCAAAAAGTCA | 110 |
| 44 | NR/15 | GGGTCATTGAGTTCA | ill |
| 45 | NR/16 | AGGTCACGAAAGGTCA | 112 |
| 46 | NR/17 | AGGTCACCCTGACCT | 113 |
| 47 | NR/18 | CTCGAGGTCATTGACCC | 114 |
| 48 | NR/19 | GAAGTGGGTCA | 115 |
| 49 | CREB/ATF/1 | ATGACGTCA | 116 |
| 50 | CREB/ATF/2 | ATGACGTCAT | 117 |
| 51 | CREB/ATF/3 | ATGATGCAAT | 118 |
| 52 | CCAAT/CEBP | TTGCGCAAT | 119 |
| 53 | AP1/2 | TGCTGAGTCAT | 120 |
| 54 | AP1/1 | TGAGTCAT | 121 |
| 55 | MAF | ATTGCTGACTCAGCA | 122 |
| 56 | ZNF317 | GAATGACAGCTGACTTCTC | 123 |
| 57 | ZNF547 | GCTAATGCAGCAGGCATAC | 124 |
| 58 | Ebox/CACGTG/1 | GTCACGTGA | 125 |
| 59 | Ebox/CACGTG/2 | GCCACGTGC | 126 |
| 60 | HIF | TACGTGC | 127 |
| 61 | CREB3/XBP1 | GATGACGTGGC | 128 |
| 62 | Ebox/CATATG | ACCATATGT | 129 |
| 63 | Ebox/CAGATGG | AAGGAAACAGATGG | 130 |
| 64 | HEN1 | GGGAGCAGCTGCGTCC | 131 |
| 65 | Ebox/CAGCTG | ACAGCTGCT | 132 |
| 66 | SCRT1 | GCAACAGGTG | 133 |
| 67 | SNAI2 | CAGGTG | 134 |
| 68 | Ebox/CACCTG | ACACCTG | 135 |
| 69 | MIES | TGACAGCTGTCA | 136 |
| 70 | TBX/1 | TCACACCTAGGTGTGA | 137 |
| 71 | TBX/2 | GTGTGAAATTTCACACC | 138 |
| 72 | TBX/3 | TCACACCTCCAAAGGTGT | 139 |
| 73 | TBX/4 | ACCTAAAAGGTGTG | 140 |
| | | AAATTCACA | |
| 74 | SREBF1 | GTGGGGTGA | 141 |
| 75 | ZNF257 | CTCTTGCCTC | 142 |
| 76 | FOX/1 | TGTGGATT | 143 |
| 77 | FOX/2 | GTAAACATGTTTAC | 144 |
| 78 | FOX/3 | GTAAACAATAAACA | 145 |
| 79 | FOX/4 | TGTTTACTTA | 146 |
| 80 | FOX/5 | GTAAACAAACAA | 147 |
| 81 | ZNF435 | TGTTAACAGAACACC | 148 |
| 82 | POU/2 | TGCATAATTAAT | 149 |
| 83 | POU/3 | CATGAATATTCA | 150 |
| 84 | OCT4 + SOX2 | CTTTGTTATGCAAA | 151 |
| 85 | POU/1 | TATGCAAAT | 152 |
| 86 | ARI5B | TTAGTATTGT | 153 |
| 87 | FOX/6 | GTAAATATTTAC | 154 |
| 88 | ARISA | AATATTGC | 155 |
| 89 | SOX/1 | AGGAGCCTTTGTT | 156 |
| 90 | DMRT1 | GATACAATGTAGC | 157 |
| 91 | SOX/2 | ATTGTT | 158 |
| 92 | SOX/3 | TGAATTGCATTCA | 159 |
| 93 | SOX/4 | AACAATTGCAGTGTT | 160 |
| 94 | SOX/5 | ATCAATTGCATTGA | 161 |
| 95 | SPI | AAAGAGGAAGTA | 162 |
| 96 | ETS/2 | AGCAGGAAGTG | 163 |
| 97 | MECP2 | CCCGGAG | 164 |
| 98 | ETS/1 | ACCGGAAGTG | 165 |
| 99 | NFAT/2 | GAAAATAATTTTCCA | 166 |
| 100 | NFAT/1 | GGAAAATTT | 167 |
| 101 | NFAC/2 | AAAGAAAAAAAG | 168 |
| 102 | NFAT/3 | TTTCCATAATGGAAA | 169 |
| 103 | IRF/3 | CCGAAACCGAAA | 170 |
| 104 | IRF/1 | CGAAACCGAAAC | 171 |
| 105 | PRDM1 | AAAGTGAAAGT | 172 |
| 106 | IRF/2 | AAAGTGAAAGTGAAAG | 173 |
| 107 | ZNF28 | TCTATTTCTTCTTGTGTCA | 174 |
| 108 | ZIM3 | GGGTTTCTGTTGCT | 175 |
| 109 | KLF/SP/2 | GGGGGGGGGCGGGGCCG | 176 |
| 110 | ZIC/2 | GGGGGGGG | 177 |
| 111 | EGR | ACGCCCACGCA | 178 |
| 112 | KLF/SP/1 | GCCACGCCCCC | 179 |
| 113 | GC-tract | GGGGAGGAGGGGGGGGGGGA | 180 |
| 114 | MZF1 | AAAGGGGACAGGCAGG | 181 |
| 115 | ZNF324 | TCAAACCATCCTTTGCTGC | 182 |
| 116 | PRDM5 | TGGAGAGCAGGG | 183 |
| 117 | EWSR1/FLI1 | GGAAGGAAGGAAGGAAG | 184 |
| 118 | ZNF436 | CAGGGAAGGCTTCCTGGAGGA | 185 |
| | | GG | |
| 119 | ZNF528 | AGGGAAGCCATTTCT | 186 |
| 120 | LEF1 | TCCTTTGATTTGC | 187 |
| 121 | ZNF354 | CCAATTCAATTATTTT | 188 |
| | | CCATTTAAT | |
| 122 | FEZF1 | TGCTCTTTT | 189 |
| 123 | ZNF274 | CATACTGGAGAGAAACCCTA | 190 |
| 124 | ZNF41 | ACAAGGGAGTAAGGCACCATG | 191 |
| | | A | |
| 125 | SMARCA5 | AATGGAATCGAATGGAATC | 192 |
| 126 | ZNF418 | TGCTTTTAGCCTCT | 193 |
| 127 | ZNF85 | AGAAAACTGAAGTAATC | 194 |
| 128 | NFI/3 | TGGCACGGTGCCA | 195 |
| 129 | SMAD | CTGTCACCTG | 196 |
| 130 | OSR2 | GCTGCTTCTGC | 197 |
| 131 | PRDM9 | AGATGGCAGCAGCATC | 198 |
| 132 | THAP1 | TGCCCGCA | 199 |
| 133 | ZSCAN3 | GCAGCAGGGCAGTTA | 200 |
| 134 | ZNF549 | TCATGAATTGGGCAGC | 201 |
| 135 | ZNF134 | CCTCACCTAATCAGTTGAAG | 202 |
| 136 | ZNF708 | TAATAAGCAGGTACAGC | 203 |
| 137 | ZNF768 | GGCTCAGAGAGGTTAAG | 204 |
| 138 | BATF | GTTTCAATATGACT | 205 |
| 139 | AIRE | TGGTTTTATTGGTTA | 206 |
| 140 | ZNF563 | GGATCCTCACTGGCAGCTG | 207 |
| 141 | ZNF667 | TGGCCTTAAAAGCTCAGC | 208 |
| 142 | ZNF554 | CTGAGCCATGTGGGGTGC | 209 |
| 143 | ZNF140 | ACCCAGCAATTCCGCTCC | 210 |
| 144 | ZFX | CCAGGCCTCGG | 211 |
| 145 | YY1 | AAAATGGCGGC | 212 |
| 146 | ZBTB6 | GGTGCTGGAGCC | 213 |
| 147 | SMAD | TGTCTGGCACC | 214 |
| 148 | HAND1 | GTCTGGCATTTGTC | 215 |
| 149 | PRDM14 | GGTTAGAGACC | 216 |
| 150 | SPZ1 | AGGGTAACAGC | 217 |
| 151 | REST/NRSF | CAGCACCATGGACAGCGC | 218 |
| 152 | ZNF332 | AGCCTGGTACAGAGCCT | 219 |
| 153 | ZNF335 | GCTGTCCTCGGCGCTGCCTG | 220 |
| 154 | ZNF382 | TAGGGTCTGTAGTGATGTCTC | 221 |
| 155 | KAISO | AATCTCGCGAGA | 222 |
| 156 | E2F/2 | GGCGGGAA | 223 |
| 157 | E2F/3 | TTTCCCGCCAAA | 224 |
| 158 | E2F/4 | GCGCCAAA | 225 |
| 159 | NFKB/3 | AAGGGAAATTCC | 226 |
| 160 | NFKB/1 | GGAAATCCCC | 227 |
| 161 | NFKB/2 | GGGGAATCCCC | 228 |
| 162 | REL-halfsite | GGGGAAT | 229 |
| 163 | ZNF282 | TTTCCCACAACACG | 230 |
| 164 | FOX/7 | TTCCCCACAC | 231 |
| 165 | RBPJ | GTGGGAAAA | 232 |
| 166 | TEAD | ACATTCCAGGCAT | 233 |
| 167 | STAT/1 | TTCTCAGGAA | 234 |
| 168 | NFAT/4 | TTTCCATGGAAA | 235 |
| 169 | BCL6/1 | TGCTTTCTAGGAATTC | 236 |
| 170 | BCL6/2 | CTTTCTAGGAA | 237 |
| 171 | STAT/2 | TTCCAGGAA | 238 |
| 172 | ZNF143 | ACCCACAATGCATT | 239 |
| 173 | ZNF143 | GCATGCTGGGAGTTGTAGT | 240 |
| 174 | EBF1 | TCCCAAGGGA | 241 |
| 175 | ZBTB48 | TAGGGACCG | 242 |
| 176 | PRDM16 | CCCAGGGG | 243 |
| 177 | ZNF423 | GCACCCAGGGGTGC | 244 |
| 178 | RUNX/2 | ACCGCAAAAACCGC | 245 |
| 179 | RUNX/1 | GTCTGTGGTT | 246 |
| 180 | SIX/1 | GAAACCTGATA | 247 |
| 181 | GFI | CTGTGATT | 248 |
| 182 | TATA | TATAAAAGG | 249 |
| 183 | MEF2 | CTATTTTTAG | 250 |
| 184 | SRF | ACCATATATGGTC | 251 |
| 185 | HLTF | CCTTAT | 252 |
| 186 | E2F/1 | AAATGGCGCCAAAA | 253 |
| 187 | HIC/1 | TGCCAACCTATGCC | 254 |
| 188 | NFI/2 | TGCCAA | 255 |
| 189 | NFI/1 | TTGGCACGGTGCCA | 256 |
| 190 | SOX/6 | AACAATGTGCAGTGTT | 257 |
| 191 | SOX/7 | ATCAATGTGCAGTGAT | 258 |
| 192 | NRF1 | TGCGCATGCGC | 259 |
| 193 | ZNF384/1 | GAAAAAA | 260 |
| 194 | ZNF384/2 | AAAAAAA | 261 |
| 195 | HOMEZ | AAACGATTAT | 262 |
| 196 | IRF/4 | AAACGAAAATCGATT | 263 |
| 197 | ZNF586 | AGGCCTAGGAGGAAAAAATG | 264 |
| 198 | ZNF24 | ATTCATTCATT | 265 |
| 199 | MFZ1 | TAGGGGGA | 266 |
| 200 | CENBP | CCCGCATACAACGAA | 267 |
| 201 | GCM | ATGCGGGTACC | 268 |
| 202 | SOX/8 | TGAATGTGCAGTCA | 269 |
| 203 | FOX/8 | CGGACACAAT | 270 |
| 204 | HD/23 | TACATGACAT | 271 |
| 205 | HD/24 | CGCTGTAAA | 272 |
| 206 | MTF1 | TTTGCACACGGCAC | 273 |
| 207 | SMAD | GTCTAGAC | 274 |
| 208 | ZNF146 | TTTCATGGCTGCATA | 275 |
| | | GTATTCC | |
| 209 | HSFY2 | TCGAAACGTTCGAA | 276 |
| 210 | HD/25 | CTAGTTAA | 277 |
| 211 | SPDEF/1 | TGGTCCCGGATCA | 278 |
| 212 | SPDEF/2 | CAGAAAGAAGTAAA | 279 |
| 213 | ZNF524 | CCCTCGAACCC | 280 |
| 214 | DMRT3 | TGTATCA | 281 |
| 215 | DDIT3 + CEBPA | TGCAATCCC | 282 |
| 216 | NR2E3 | AAGCTT | 283 |
| 217 | ZBTB14 | GAGCGCGC | 284 |
| 218 | ZNF57 | TGCGGCA | 285 |
| 219 | ZNF250 | ACCATGCTGTTTTGATTA | 286 |
| 220 | ZNF490 | GGGTCTCTTGAAGGCAGCATA | 287 |
| 221 | ZNF53 | ATCCAC | 288 |
| 222 | ZNF784 | GTACCTACCT | 289 |
| 223 | GMEB2/1 | ACGTAACCACGTA | 290 |
| 224 | LIN54 | TTTGAAT | 291 |
| 225 | PRDM4 | TTTCAAGGCCCCC | 292 |
| 226 | ZBTB49 | TTTCGCCTGGCACGTC | 293 |
| 227 | ZNF136 | GCTGGATATAGTATTC | 294 |
| | | TTGGTT | |
| 228 | ZNF431 | ACCAACCTAAGACAGG | 295 |
| 229 | ZNF306 | GGTCTAGCCTCG | 296 |
| 230 | ZNF713 | AGAAAAATGCCACGAA | 297 |
| 231 | ZNF85 | GTGGTATATCCATACAATGGA | 298 |
| 232 | ZNF652 | AAAGGGTTAA | 299 |
| 233 | P53-like/2 | ACATGTCTATGACATGT | 300 |
| 234 | P53-like/3 | ACATGTCCATGGACATGT | 301 |
| 235 | ZNF232 | GTTAAATGTAGATTAA | 302 |
| 236 | ZNF410 | CATCCCATAATA | 303 |
| 237 | ZBED1 | TATCGCGACAT | 304 |
| 238 | PROX1 | AAGACGCCTT | 305 |
| 239 | MBD2 | GGGCCGGAG | 306 |
| 240 | OVOL1 | TGTAACTGT | 307 |
| 241 | EVI1/MECOM | AGATAAGATAAGA | 308 |
| 242 | GATA | CTTATCTGTC | 309 |
| 243 | HIC/2 | GGTTGCC | 310 |
| 244 | ZFN121 | CTGGGCAACATAGCAAGAC | 311 |
| 245 | RFX/2 | GTTGCTAGGCAAC | 312 |
| 246 | RFX/3 | GCTGTTGCCAGGC | 313 |
| 247 | RFX/1 | GTTGCCATGGCAAC | 314 |
| 248 | NR/20 | GAACATTGTGTTCC | 315 |
| 249 | HSF | TTCTAGAACGTTC | 316 |
| 250 | SMARCA1 | CAAGAAGAAT | 317 |
| 251 | ZNF680 | ATGTCCAAGAAGAATGAGG | 318 |
| 252 | MYB/5 | GGCAGTTG | 319 |
| 253 | MYB/1 | ACCGTTAACGG | 320 |
| 254 | MYB/2 | CCGTTAAAACCGTT | 321 |
| 255 | MYB/3 | ACCGTTAAACGG | 322 |
| 256 | MYB/4 | AACCGTTA | 323 |
| 257 | P53-like/l | ACATGCCCAGACATGC | 324 |
| 258 | GRHL | GAAAAACCGGTT | 325 |
| 259 | TFCP2 | CCGGTTCAAACCGGT | 326 |
| 260 | ZNF329 | CTGGATCCAGCCATGCCTGA | 327 |
| 261 | GLIS | GACCCCCCACGAAG | 328 |
| 262 | GL1 | TCGGTGGTCG | 329 |
| 263 | TFAP2/2 | GCCCTGAGGGC | 330 |
| 264 | TFAP2/1 | GCCTGAGGC | 331 |
| 265 | CTCF | CCACCAGGGGGCGC | 332 |
| 266 | INSM1 | TGTCAGGGGGCA | 333 |
| 267 | ZBTB7A | GGGGTCT | 334 |
| 268 | PLAG1 | GGGGCCCAAGGGGG | 335 |
| 269 | ZNF320 | GTGGGACCAGGGGGCCAGT | 336 |
| 270 | ZNF331 | GGCTGGGCTCAGCTGGGTG | 337 |
| 271 | ZNF449 | GGTTGGGCT | 338 |
| 272 | ZKSCAN1 | GGACCTACTGTGTG | 339 |
| 273 | ZIC | ACCCCCCGCTGTG | 340 |
| 274 | HINFP1/1 | AACGTCCGCG | 341 |
| 275 | HINFP1/2 | GCGGACGTTGCAACGTCCGC | 342 |
| 276 | HINFP1/3 | GCGGACGTTCAACGTCCGC | 343 |
| 277 | GMEB2/2 | TACGTAA | 344 |
| 278 | GMEB2/3 | ACGTAACTGACGTA | 345 |
| 279 | FOX/9 | AATGACACAGCG | 346 |
| 280 | SIX/2 | ATAGGGTATCACT | 347 |
| 281 | PAX/1 | GTCACGCATGACTG | 348 |
| 282 | PAX/2 | GGGCAGCAGAGCGTGAC | 349 |
| 283 | PAX-halfsite | GTCACG | 350 |
| 284 | AHR | TGCGTG | 351 |
| 285 | KLF/SP/3 | CAGGGGGTG | 352 |
| 286 | ZSCAN4 | GCACACACTGAAA | 353 |
| 287 | SHAPE_ | AACCCGGAAGTGAACC | 354 |
| | positive_ | CGGAAGTG | |
| | controL_ELF2X | | |
| 288 | SHAPE_ | GCACTTTGTACAGGGT | 355 |
| | negative_ | GCCATCGG | |
| | control_1 | | |
| 289 | SHAPE_ | GTTTCTGAACTCTCAG | 356 |
| | negative_ | | |
| | control_2 | ATAGTGGG | |
| 290 | SHAPE_ | GCGGTCCAACTTAGGC | 357 |
| | negative_ | GTAAACCT | |
| | control_3 | | |
| 291 | SHAPE_ | CGATGCTACCTACTCA | 358 |
| | negative_ | GACCCACC | |
| | control_4 | | |
| 292 | SHAPE_ | ATCCCAGCTGGTTCT | 359 |
| | negative_ | TGGCGTTCT | |
| | control_5 | | |

### Example 5: Pooled Screening of Mutagenized ELF(2×) Insertion Motifs for SHAPE-Mediated Tunable Protein Expression

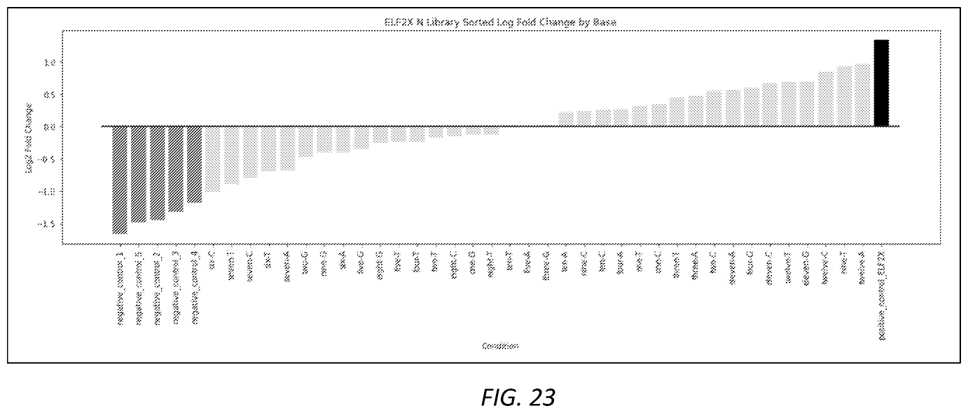

We explored the insertion of mutagenized ELF(2×) insertion motifs via prime editing to achieve tunable target protein expression. To do so, we used the IL2RA promoter due to our previous success in activating IL2RA protein expression and the ability to sort for its expression as a cell surface marker (FIG. 20). We mutagenized positions across the ELF motif by introducing single base substitutions at the same positions of each copy within the ELF(2×) insertion sequence. We mutated the reference bases to all other possible bases at all positions across the 12 bp ELF motif for a total of 36 unique mutagenized ELF motifs. Additionally, we included a positive control wild type ELF(2×) motif which we had previously shown to activate IL2RA protein expression, along with 5 negative control sequences (24 bp) created with a random DNA generator. All mutagenized ELF(2×) motifs and control sequences were inserted at the same position within the IL2RA promoter using the pegRNA spacer sequence and ngRNA described previously (Table B, C). The pegRNA constructs encoding all mutagenized ELF(2×) motifs and control sequences, in addition to the ngRNA, were pooled and double-electroporated into K562 with the electroporation events separated by 72 hours. Seven days after the second electroporation event, the K562 cells were sorted based on IL2RA protein expression into negative and positive populations. Genomic DNA was harvested from both the IL2RA negative and positive populations and targeted amplicon sequencing was performed at the prime editing insertion location. Each mutagenized ELF(2×) motif was counted within the sequencing reads in both the negative and positive samples, normalized based on total read count in each sample, and an enrichment score was calculated (FIG. 23). We found that our positive control wild-type ELF(2×) motif exhibited the highest positive enrichment (FIG. 23, black), whereas the 5 negative controls showed the most negative enrichment (FIG. 23, dark grey). The different ELF motif single-base variants ((FIG. 23, light grey) exhibited varying degrees of enrichment, suggesting the ability of SHAPE to tune target protein expression by mutagenizing transcription factor motifs.

TABLE F
Single-base mutagenized ELF motifs for
tuning of target gene activation

| Motif name | Motif sequence | SEQ ID NO: |
|---|---|---|
| ELF 1 mutagenesis | BACCCGGAAGTG | 360 |
| ELF 2 mutagenesis | ABCCCGGAAGTG | 361 |
| ELF 3 mutagenesis | AADCCGGAAGTG | 362 |
| ELF 4 mutagenesis | AACDCGGAAGTG | 363 |
| ELF 5 mutagenesis | AACCDGGAAGTG | 364 |
| ELF 6 mutagenesis | AACCCHGAAGTG | 365 |
| ELF 7 mutagenesis | AACCCGHAAGTG | 366 |
| ELF 8 mutagenesis | AACCCGGBAGTG | 367 |
| ELF 9 mutagenesis | AACCCGGABGTG | 368 |
| ELF 10 mutagenesis | AACCCGGAAHTG | 369 |
| ELF 11 mutagenesis | AACCCGGAAGVG | 370 |
| ELF 12 mutagenesis | AACCCGGAAGTH | 371 |

Taken together, these results show that it is possible to use precision genome engineering via prime editing to install functional transcription factor binding sites, to ultimately promote heritable gene activation.

TABLE G
SNPs that affect microRNA binding sites which are associated with autoimmune diseases.
Table 2 taken from De Almeida et al, Frontiers in Genetics 2018, 9:139. doi: 10.3389/fgene.2018.00139.
eCollection 2018.

| | | | | | | | | | | | Effect | | eQTL |
|---|---|---|---|---|---|---|---|---|---|---|---|---|---|
| | | | Ref. | Minor | | | P- | | Target | | on | | (effect |
| miRSNP | chr | pos_bp(hg38) | allele | allele | MAF | Disease | value | OR | gene | miRNA | miRNA | NBS | direction) |
| rs7559479 | 2 | 102452327 | A | G | 0.22 | Crohn's | 2.2e−10 | 1.15 | IL18RAP | hsa- | C | 0.79 | Yes (up) |
| | | | | | | disease | | | | miR- | | | |
| | | | | | | | | | | 3156-3p | | | |
| | | | | | | Celiac | 1.4e−16 | 1.19 | | hsa- | C | 0.86 | |
| | | | | | | disease | | | | miR- | | | |
| | | | | | | | | | | 4301 | | | |
| | | | | | | Inflammatory | NA | NA | | hsa- | C | 0.92 | |
| | | | | | | bowel | | | | miR- | | | |
| | | | | | | disease | | | | 136 | | | |
| rs7603250 | 2 | 102452374 | A | T | 0.22 | Crohn's | 1.6e−10 | 0.8 | IL18RAP | hsa- | C | 0.94 | Yes (up) |
| | | | | | | disease | | | | miR- | | | |
| | | | | | | | | | | 455- | | | |
| | | | | | | | | | | 3p | | | |
| | | | | | | Inflammatory | 1.8e−09 | 0.8 | | | | | |
| | | | | | | bowel | | | | | | | |
| | | | | | | disease | | | | | | | |
| | | | | | | Celiac | NA | NA | | | | | |
| | | | | | | disease | | | | | | | |
| rs3732421 | 3 | 119431242 | A | G | 0.16 | Multiple | 3.5e−12 | 0.85 | TMEM39A | hsa- | D | 0.72 | No |
| | | | | | | sclerosis | | | | miR- | | | |
| | | | | | | | | | | 449b- | | | |
| | | | | | | | | | | 3p | | | |
| | | | | | | Primary | 1.7e−11 | 0.72 | | hsa- | D | 0.82 | |
| | | | | | | biliary | | | | miR- | | | |
| | | | | | | cirrhosis | | | | 4691- | | | |
| | | | | | | | | | | 3p | | | |
| | | | | | | | | | | hssa- | D | 0.92 | |
| | | | | | | | | | | miR- | | | |
| | | | | | | | | | | 449b | | | |
| rs57271503 | 3 | 119525746 | G | A | 0.16 | Multiple | 4.1e−15 | 0.84 | CD80 | hsa- | C | 0.71 | No |
| | | | | | | sclerosis | | | | miR- | | | |
| | | | | | | | | | | 769- | | | |
| | | | | | | | | | | 5p | | | |
| | | | | | | Primary | 9.4e−13 | 0.71 | | hsa- | C | 0.80 | |
| | | | | | | biliary | | | | miR- | | | |
| | | | | | | cirrhosis | | | | 4802- | | | |
| | | | | | | | | | | 5p | | | |
| | | | | | | | | | | hsa- | C | 0.71 | |
| | | | | | | | | | | miR- | | | |
| | | | | | | | | | | 769- | | | |
| | | | | | | | | | | 5p | | | |
| rs1054037 | 4 | 102631552 | T | C | 0.49 | Primary | 8.3e−10 | 0.82 | MANBA | hsa- | D | 0.90 | Yes |
| | | | | | | biliary | | | | miR- | | | (down/ |
| | | | | | | cirrhosis | | | | 660 | | | up) |
| | | | | | | Multiple | NA | NA | | hsa- | D | 0.95 | |
| | | | | | | sclerosis | | | | miR- | | | |
| | | | | | | | | | | 5591- | | | |
| | | | | | | | | | | 3p | | | |
| | | | | | | | | | | hsa- | D | 0.82 | |
| | | | | | | | | | | miR- | | | |
| | | | | | | | | | | 660- | | | |
| | | | | | | | | | | 5p | | | |
| | | | | | | | | | | hsa- | C | 0.71 | |
| | | | | | | | | | | miR- | | | |
| | | | | | | | | | | 7151- | | | |
| | | | | | | | | | | 5p | | | |
| | | | | | | | | | | hsa- | C | 0.94 | |
| | | | | | | | | | | miR- | | | |
| | | | | | | | | | | 3686 | | | |
| rs4013 | 4 | 102631656 | T | C | 0.51 | Primary | NA | NA | MANBA | hsa- | C | 0.76 | Yes |
| | | | | | | biliary | | | | miR- | | | (down/ |
| | | | | | | cirrhosis | | | | 4742- | | | up) |
| | | | | | | | | | | 3p | | | |
| | | | | | | Multiple | NA | NA | | hsa- | C | 0.91 | |
| | | | | | | sclerosis | | | | miR- | | | |
| | | | | | | | | | | 4778- | | | |
| | | | | | | | | | | 5p | | | |
| | | | | | | | | | | hsa- | C | 0.82 | |
| | | | | | | | | | | miR- | | | |
| | | | | | | | | | | 630 | | | |
| rs1054029 | 4 | 102631896 | A | G | 0.51 | Primary | NA | NA | MANBA | hsa- | D | 0.80 | Yes |
| | | | | | | biliary | | | | miR- | | | (down/ |
| | | | | | | cirrhosis | | | | 124- | | | up) |
| | | | | | | | | | | 5p | | | |
| | | | | | | Multiple | NA | NA | | hsa- | D | 0.99 | |
| | | | | | | sclerosis | | | | miR- | | | |
| | | | | | | | | | | 4255- | | | |
| | | | | | | | | | | hsa- | D | 0.74 | |
| | | | | | | | | | | miR- | | | |
| | | | | | | | | | | 4766- | | | |
| | | | | | | | | | | 5p | | | |
| | | | | | | | | | | hsa- | D | 0.90 | |
| | | | | | | | | | | miR- | | | |
| | | | | | | | | | | 124 | | | |
| | | | | | | | | | | hsa- | D | 0.77 | |
| | | | | | | | | | | miR- | | | |
| | | | | | | | | | | 33a | | | |
| rs39602 | 5 | 97028750 | G | C | 0.41 | Ankylosing | NA | NA | LNPEP | hsa- | D | 0.99 | Yes |
| | | | | | | spondylitis | | | | miR- | | | (down) |
| | | | | | | | | | | 6800- | | | |
| | | | | | | | | | | 5p | | | |
| | | | | | | Crohn's | 6.9e−11 | 1.1 | | | | | |
| | | | | | | disease | | | | | | | |
| | | | | | | Juvenile | NA | NA | | | | | |
| | | | | | | idiopathic | | | | | | | |
| | | | | | | arthritis | | | | | | | |
| rs2070197 | 7 | 128948946 | T | C | 0.1 | Systemic | 3.0e−40 | 1.7 | IRF5 | hsa- | D | 0.91 | Yes |
| | | | | | | lupus | | | | miR- | | | (up) |
| | | | | | | erythematosus | | | | 3136- | | | |
| | | | | | | | | | | 3p | | | |
| | | | | | | Primary | | | | hsa- | D | 0.89 | |
| | | | | | | biliary | | | | miR- | | | |
| | | | | | | cirrhosis | | | | 7155- | | | |
| | | | | | | | | | | 3p | | | |
| rs10114470 | 9 | 114785492 | C | T | 0.33 | Crohn's | 1.5e−15 | 1.1 | TNFSF15 | hsa- | D | 0.84 | Yes |
| | | | | | | disease | | | | miR- | | | (up) |
| | | | | | | | | | | 376a- | | | |
| | | | | | | | | | | 3p | | | |
| | | | | | | Inflammatory | 1.5e−15 | 0.8 | | hsa- | D | 0.74 | |
| | | | | | | bowel | | | | miR- | | | |
| | | | | | | disease | | | | 4753- | | | |
| | | | | | | | | | | 5p | | | |
| rs3088081 | 9 | 136375697 | A | G | 0.42 | Inflammatory | 1.9e−22 | 0.8 | SNAPC4 | hsa- | C | 0.80 | Yes |
| | | | | | | bowel | | | | miR- | | | (down) |
| | | | | | | disease | | | | 3661 | | | |
| | | | | | | Crohn's | 2.1e−17 | 0.8 | | | | | |
| | | | | | | disease | | | | | | | |
| | | | | | | Ulcerative | 1.0e−11 | 0.8 | | | | | |
| | | | | | | colitis | | | | | | | |
| rs9943 | 13 | 39752145 | A | G | 0.34 | Juvenile | NA | NA | COG6 | hsa- | C | 0.98 | Yes |
| | | | | | | idiopathic | | | | miR- | | | (down) |
| | | | | | | arthritis | | | | 628- | | | |
| | | | | | | Rheumatoid | NA | NA | | 5p | | | |
| | | | | | | arthritis | | | | | | | |
| rs3839999 | 13 | 99385548 | AT | A | 0.22 | Crohn's | NA | NA | UBAC2 | hsa- | C | 0.93 | No |
| | | | | | | disease | | | | miR- | | | |
| | | | | | | Inflammatory | NA | NA | | 365a- | | | |
| | | | | | | bowel | | | | 3p | | | |
| | | | | | | disease | | | | | | | |
| rs907091 | 17 | 39765489 | C | T | 0.51 | Primary | 1.0e−10 | 0.7 | IKZF3 | hsa- | D | 0.97 | Yes |
| | | | | | | biliary | | | | miR- | | | (down) |
| | | | | | | cirrhosis | | | | 4497 | | | |
| | | | | | | Type 1 | NA | NA | | hsa- | C | 0.85 | |
| | | | | | | diabetes | | | | miR- | | | |
| | | | | | | mellitus | | | | 3649 | | | |
| | | | | | | Ulcerative | 8.1e−09 | 0.8 | | hsa- | C | 0.92 | |
| | | | | | | colitis | | | | miR- | | | |
| | | | | | | | | | | 4518 | | | |
| | | | | | | Inflammatory | 3.3e−13 | 0.8 | | hsa- | C | 0.99 | |
| | | | | | | bowel | | | | miR- | | | |
| | | | | | | disease | | | | 330- | | | |
| | | | | | | | | | | 5p | | | |
| | | | | | | | | | | hsa- | C | 0.86 | |
| | | | | | | | | | | miR- | | | |
| | | | | | | | | | | 518c | | | |
| | | | | | | | | | | hsa- | C | 0.84 | |
| | | | | | | | | | | miR- | | | |
| | | | | | | | | | | 4314 | | | |
| | | | | | | | | | | hsa- | C | 0.99 | |
| | | | | | | | | | | miR- | | | |
| | | | | | | | | | | 326 | | | |
| | | | | | | | | | | hsa- | C | 0.73 | |
| | | | | | | | | | | miR- | | | |
| | | | | | | | | | | 3192 | | | |
| rs16940681 | 17 | 45834793 | G | C | 0.24 | Primary | NA | NA | CRHR1 | hsa- | D | 0.84 | Yes (up) |
| | | | | | | biltary | | | | miR- | | | |
| | | | | | | cirrhosis | | | | 6740-5p | | | |
| | | | | | | Type 1 | NA | NA | | | | | |
| | | | | | | diabetes | | | | | | | |
| | | | | | | mellitus | | | | | | | |
| rs2316765 | 17 | 45835088 | T | C | 0.24 | Primary | NA | NA | CRHR1 | hsa- | D | 0.73 | Yes (up) |
| | | | | | | biltary | | | | miR- | | | |
| | | | | | | cirrhosis | | | | 3192 | | | |
| | | | | | | Type 1 | NA | NA | | hsa- | D | 0.97 | |
| | | | | | | diabetes | | | | miR- | | | |
| | | | | | | mellitus | | | | 30c-2- | | | |
| | | | | | | | | | | 3p | | | |
| | | | | | | | | | | hsa- | D | 0.90 | |
| | | | | | | | | | | miR- | | | |
| | | | | | | | | | | 30c-1- | | | |
| | | | | | | | | | | 3p | | | |
| rs878886 | 17 | 45835124 | C | G | 0.24 | Primary | NA | NA | CRHR1 | hsa- | D | 0.96 | Yes (up) |
| | | | | | | biltary | | | | miR- | | | |
| | | | | | | cirrhosis | | | | 4685- | | | |
| | | | | | | Type 1 | NA | NA | | 5p | | | |
| | | | | | | diabetes | | | | hsa- | D | 0.71 | |
| | | | | | | mellitus | | | | miR- | | | |
| | | | | | | | | | | 1915- | | | |
| | | | | | | | | | | 3p | | | |
| | | | | | | | | | | hsa- | D | 0.86 | |
| | | | | | | | | | | miR- | | | |
| | | | | | | | | | | 3918 | | | |
| | | | | | | | | | | hsa- | D | 0.76 | |
| | | | | | | | | | | miR- | | | |
| | | | | | | | | | | 7160- | | | |
| | | | | | | | | | | 3p | | | |
| rs878887 | 17 | 45835216 | C | T | 0.24 | Primary | NA | NA | CRHR1 | hsa- | C | 0.85 | Yes (up) |
| | | | | | | biltary | | | | miR- | | | |
| | | | | | | cirrhosis | | | | 198 | | | |
| | | | | | | Type 1 | NA | NA | | hsa- | D | 0.88 | |
| | | | | | | diabetes | | | | miR- | | | |
| | | | | | | mellitus | | | | 3186- | | | |
| | | | | | | | | | | 5p | | | |
| | | | | | | | | | | hsa- | C | 0.92 | |
| | | | | | | | | | | miR- | | | |
| | | | | | | | | | | 136 | | | |
| rs878888 | 17 | 45835269 | A | G | 0.24 | Primary | NA | NA | CRHR1 | hsa- | C | 0.70 | Yes (up) |
| | | | | | | biltary | | | | miR- | | | |
| | | | | | | cirrhosis | | | | 5708 | | | |
| | | | | | | Type 1 | NA | NA | | hsa- | D | 0.72 | |
| | | | | | | diabetes | | | | miR- | | | |
| | | | | | | mellitus | | | | 1226- | | | |
| | | | | | | | | | | 5p | | | |
| rs4640231 | 17 | 45835420 | G | C | 0.24 | Primary | NA | NA | CRHR1 | hsa-miR- | D | 0.81 | Yes (up) |
| | | | | | | biltary | | | | 6841-5p | | | |
| | | | | | | cirrhosis | | | | | | | |
| | | | | | | Type 1 | NA | NA | | hsa-miR- | D | 0.77 | |
| | | | | | | diabetes | | | | 6755-5p | | | |
| | | | | | | mellitus | | | | | | | |
| rs54482334 | 17 | 45835464 | T | C | 0.24 | Primary | NA | NA | CRHR1 | hsa-miR- | D | 0.93 | Yes (up) |
| | | | | | | biltary | | | | 6890-5p | | | |
| | | | | | | cirrhosis | | | | | | | |
| | | | | | | Type 1 | NA | NA | | hsa-miR- | D | 0.94 | |
| | | | | | | diabetes | | | | 6742-5p | | | |
| | | | | | | mellitus | | | | | | | |
| | | | | | | | | | | hsa-miR- | D | 0.77 | |
| | | | | | | | | | | 4722-5p | | | |
| | | | | | | | | | | hsa-miR- | D | 0.75 | |
| | | | | | | | | | | 6796-5p | | | |
| | | | | | | | | | | hsa-miR- | D | 0.80 | |
| | | | | | | | | | | 4459 | | | |
| rs12373168 | 17 | 45846971 | A | C | 0.24 | Primary | NA | NA | SPPL2C | hsa-miR- | D | 0.87 | No |
| | | | | | | biltary | | | | 33b-3p | | | |
| | | | | | | cirrhosis | | | | | | | |
| | | | | | | Type 1 | NA | NA | | hsa-miR- | D | 0.87 | |
| | | | | | | diabetes | | | | 519e-3p | | | |
| | | | | | | mellitus | | | | | | | |
| rs60474474 | 18 | 12792737 | C | T | 0.14 | Inflammatory | 1.4e−10 | 1.1 | PTPN2 | hsa-miR- | D | 0.99 | No |
| | | | | | | bowel | | | | 4290 | | | |
| | | | | | | disease | | | | | | | |
| | | | | | | Crohn's | 2.2e−12 | 1.2 | | | | | |
| | | | | | | disease | | | | | | | |
| | | | | | | Type 1 | NA | NA | | | | | |
| | | | | | | diabetes | | | | | | | |
| | | | | | | mellitus | | | | | | | |
| | | | | | | Celiac | NA | NA | | | | | |
| | | | | | | disease | | | | | | | |
| | | | | | | Juvenile | NA | NA | | | | | |
| | | | | | | idiopathic | | | | | | | |
| | | | | | | arthritis | | | | | | | |
| rs45450798 | 18 | 12792941 | C | G | 0.14 | Inflammatory | 2.0e−10 | 0.8 | PTPN2 | hsa-miR- | D | 0.73 | No |
| | | | | | | bowel | | | | 4531 | | | |
| | | | | | | disease | | | | | | | |
| | | | | | | Crohn's | 2.9e−12 | 0.8 | | | | | |
| | | | | | | disease | | | | | | | |
| | | | | | | Type 1 | NA | NA | | | | | |
| | | | | | | diabetes | | | | | | | |
| | | | | | | mellitus | | | | | | | |
| | | | | | | Celiac | NA | NA | | | | | |
| | | | | | | disease | | | | | | | |
| | | | | | | Juvenile | NA | NA | | | | | |
| | | | | | | idiopathic | | | | | | | |
| | | | | | | arthritis | | | | | | | |
| rs9950174 | 18 | 69846569 | T | C | 0.53 | Psoriasis | NA | NA | CD226 | hsa-miR- | D | 0.85 | Yes |
| | | | | | | | | | | 5189-3p | | | (up) |
| rs727088 | 18 | 69863203 | G | A | 0.53 | Inflammatory | 4.6e−9 | 1.08 | CD226 | hsa-miR- | D | 0.75 | Yes |
| | | | | | | bowel | | | | 513a-3p | | | (up) |
| | | | | | | disease | | | | | | | |
| | | | | | | Ulcerative | 1.9e−8 | 1.1 | | hsa-miR- | D | 0.75 | |
| | | | | | | colitis | | | | 181c | | | |
| | | | | | | Psoriasis | NA | NA | | | | | |
| rs571689 | 19 | 48704297 | C | T | 0.47 | Crohn's | 7.3e−09 | 1.1 | FUT2 | hsa-miR- | D | 0.89 | Yes |
| | | | | | | disease | | | | 648 | | | (down) |
| | | | | | | Type 1 | NA | NA | | hsa-miR- | C | 0.92 | |
| | | | | | | diabetes | | | | 552-3p | | | |
| | | | | | | mellitus | | | | | | | |
| rs570794 | 19 | 48704394 | T | C | 0.47 | Crohn's | 1.0e−08 | 0.8 | FUT2 | hsa-miR- | D | 0.71 | Yes |
| | | | | | | disease | | | | 4430 | | | (down) |
| | | | | | | Type 1 | NA | NA | | hsa-miR- | C | 0.79 | |
| | | | | | | diabetes | | | | 1295b- | | | |
| | | | | | | mellitus | | | | 5p | | | |
| | | | | | | | | | | hsa-miR- | C | 0.95 | |
| | | | | | | | | | | 4463 | | | |
| | | | | | | | | | | hsa-miR- | C | 0.94 | |
| | | | | | | | | | | 1912 | | | |
| rs507766 | 19 | 48705286 | T | C | 0.47 | Crohn's | 1.4e−08 | 0.8 | FUT2 | hsa-miR- | C | 0.83 | Yes |
| | | | | | | disease | | | | 136-5p | | | (down) |
| | | | | | | Type 1 | NA | NA | | hsa- | C | 0.80 | |
| | | | | | | diabetes | | | | miR- | | | |
| | | | | | | mellitus | | | | 675 | | | |
| | | | | | | | | | | hsa-miR- | C | 0.92 | |
| | | | | | | | | | | 887 | | | |
| | | | | | | | | | | hsa-miR- | C | 0.73 | |
| | | | | | | | | | | 410 | | | |
| | | | | | | | | | | hsa-miR- | C | 0.75 | |
| | | | | | | | | | | 191 | | | |
| rs506897 | 19 | 48705372 | G | C | 0.47 | Crohn's | 7.3e−09 | 1.1 | FUT2 | hsa-miR- | C | 0.81 | Yes |
| | | | | | | disease | | | | 4530 | | | (down) |
| | | | | | | Type 1 | NA | NA | | | | | |
| | | | | | | diabetes | | | | | | | |
| | | | | | | mellitus | | | | | | | |
| rs503279 | 19 | 48705753 | T | C | 0.47 | Crohn's | 6.7e−09 | 0.8 | FUT2 | hsa-miR- | C | 0.86 | Yes |
| | | | | | | disease | | | | 675-3p | | | (down) |
| | | | | | | Type 1 | NA | NA | | | | | |
| | | | | | | diabetes | | | | | | | |
| | | | | | | mellitus | | | | | | | |
| rs1056441 | 20 | 63738996 | C | T | 0.28 | Crohn's | 5.4e−11 | 0.8 | LIME1 | hsa-miR- | C | 0.97 | Yes |
| | | | | | | disease | | | | 4745-3p | | | (down) |
| | | | | | | Inflammatory | 1.5e−15 | 0.8 | | hsa-miR- | C | 0.97 | |
| | | | | | | bowel disease | | | | 1538 | | | |
| | | | | | | | | | | hsa-miR- | C | 0.87 | |
| | | | | | | | | | | 4467 | | | |
| | | | | | | | | | | hsa-miR- | C | 0.78 | |
| | | | | | | | | | | 6770-3p | | | |
| | | | | | | | | | | hsa-miR- | D | 0.82 | |
| | | | | | | | | | | 3940-3p | | | |
| | | | | | | | | | | hsa-miR- | D | 0.94 | |
| | | | | | | | | | | 762 | | | |
| rs7444 | 22 | 21622645 | T | C | 0.2 | Systemic | 1.8e−22 | 1.27 | UBE2L3 | hsa-miR- | D | 0.90 | Yes |
| | | | | | | lupus | | | | 4741 | | | (up) |
| | | | | | | erythematosus | | | | | | | |
| | | | | | | Crohn's | 1.3e−12 | 1.1 | | hsa-miR- | D | 0.89 | |
| | | | | | | disease | | | | 4763-3p | | | |
| | | | | | | Inflammatory | 6.9e−10 | 0.8 | | hsa-miR- | D | 0.86 | |
| | | | | | | bowel disease | | | | 3918 | | | |
| | | | | | | | | | | hsa-miR- | D | 0.92 | |
| | | | | | | | | | | 1207-5p | | | |
| rs7445 | 22 | 21622758 | C | T | 0.19 | Crohn's | 6.3e−09 | 1.1 | UBE2L3 | hsa-miR- | C | 0.93 | Yes |
| | | | | | | disease | | | | 3064-5p | | | (up) |
| | | | | | | Inflammatory | 3.5e−10 | 1.1 | | | | | |
| | | | | | | bowel disease | | | | | | | |
| | | | | | | Systemic | 1.0e−12 | 1.27 | | | | | |
| | | | | | | lupus | | | | | | | |
| | | | | | | erythematosus | | | | | | | |

NA, P-value was not available in the source study but this miRSNP (SNPs that can affect miRNA binding) is in linkage disequilibrium (r² ≥ 0.8) with the most associated SNP reported; P-value obtained from each study. Bold: reported as associated in the original study. C, creates a binding site; D, disrupts a binding site; chr, chromosome; MAF, global minor allele frequency from the 1000 Genomes project; up, upregulated effect; down, downregulated effect; pos bp, position in base pair; OR, odds ratio. OR was calculated based on the minor allele of the respective variant; eQTL, expression quantitative trait locus; NBC, naïve Bayes combined score.
Table 2 and legend adapted from De Almeida et al, Frontiers in Genetics 9:139 2018 (PMID 29755505)

TABLE H
Organ, tissue or cell-specific miRNAs from the miRWalk 2.0 database

| Organ/Tissue/Cell type | miRNA | MIMATid | PMID |
|---|---|---|---|
| Adherens Junctions | hsa-miR-1 | MIMAT0000416 | 23142026 |
| Adipocytes | bta-miR-29a | MIMAT0003518 | 17652184 |
| Adipocytes | bta-miR-29c | MIMAT0003829 | 17652184 |
| Adipocytes | cfa-miR-29a | MIMAT0006626 | 17652184 |
| Adipocytes | cfa-miR-29c | MIMAT0006705 | 17652184 |
| Adipocytes | dre-miR-29a | MIMAT0001802 | 17652184 |
| Adipocytes | hsa-miR-125a-5p | MIMAT0000443 | 19011087 |
| Adipocytes | hsa-miR-125b-5p | MIMAT0000423 | 19011087 |
| Adipocytes | hsa-miR-132-3p | MIMAT0000426 | 19819989 |
| Adipocytes | hsa-miR-138-5p | MIMAT0000430 | 20486779 |
| Adipocytes | hsa-miR-141-3p | MIMAT0000432 | 20005803 |
| Adipocytes | hsa-miR-143-3p | MIMAT0000435 | 15504739 |
| Adipocytes | hsa-miR-199a-5p | MIMAT0000231 | 19011087 |
| Adipocytes | hsa-miR-199a-5p | MIMAT0000231 | 22441842 |
| Adipocytes | hsa-miR-200a-3p | MIMAT0000682 | 20005803 |
| Adipocytes | hsa-miR-200b-3p | MIMAT0000318 | 20005803 |
| Adipocytes | hsa-miR-200c-3p | MIMAT0000617 | 20005803 |
| Adipocytes | hsa-miR-21-5p | MIMAT0000076 | 21381024 |
| Adipocytes | hsa-miR-26a-5p | MIMAT0000082 | 19011087 |
| Adipocytes | hsa-miR-27b-3p | MIMAT0000419 | 19800867 |
| Adipocytes | hsa-miR-346 | MIMAT0000773 | 19011087 |
| Adipocytes | hsa-miR-429 | MIMAT0001536 | 20005803 |
| Adipocytes | hsa-miR-519d-3p | MIMAT0002853 | 20057369 |
| Adipocytes | hsa-miR-637 | MIMAT0003307 | 21880893 |
| Adipocytes | mmu-miR-17-5p | MIMAT0000649 | 18287052 |
| Adipocytes | mmu-miR-204-5p | MIMAT0000237 | 20039258 |
| Adipocytes | mmu-miR-211-5p | MIMAT0000668 | 20039258 |
| Adipocytes | mmu-miR-29a-3p | MIMAT0000535 | 17652184 |
| Adipocytes | mmu-miR-29b-3p | MIMAT0000127 | 17652184 |
| Adipocytes | mmu-miR-29c-3p | MIMAT0000536 | 17652184 |
| Adipocytes | mmu-miR-448-3p | MIMAT0001533 | 20719859 |
| Adipocytes | ppy-miR-29a | MIMAT0002363 | 17652184 |
| Adipocytes | ppy-miR-29c | MIMAT0015735 | 17652184 |
| Adipocytes | ptr-miR-29a | MIMAT0002364 | 17652184 |
| Adipocytes | ptr-miR-29c | MIMAT0008082 | 17652184 |
| Adipocytes | rno-miR-29a-3p | MIMAT0000802 | 17652184 |
| Adipocytes | rno-miR-29b-3p | MIMAT0000801 | 17652184 |
| Adipocytes | rno-miR-29c-3p | MIMAT0000803 | 17652184 |
| Adipocytes | ssc-miR-29a | MIMAT0013870 | 17652184 |
| Adipocytes | ssc-miR-29c | MIMAT0002166 | 17652184 |
| Adipocytes | xtr-miR-29a | MIMAT0003656 | 17652184 |
| Adipose Tissue | hsa-miR-132-3p | MIMAT0000426 | 19819989 |
| Adipose Tissue | hsa-miR-141-3p | MIMAT0000432 | 20005803 |
| Adipose Tissue | hsa-miR-196a-5p | MIMAT0000226 | 19063684 |
| Adipose Tissue | hsa-miR-200a-3p | MIMAT0000682 | 20005803 |
| Adipose Tissue | hsa-miR-200b-3p | MIMAT0000318 | 20005803 |
| Adipose Tissue | hsa-miR-200c-3p | MIMAT0000617 | 20005803 |
| Adipose Tissue | hsa-miR-21-5p | MIMAT0000076 | 21381024 |
| Adipose Tissue | hsa-miR-21-5p | MIMAT0000076 | 19816956 |
| Adipose Tissue | hsa-miR-26a-5p | MIMAT0000082 | 18197755 |
| Adipose Tissue | hsa-miR-429 | MIMAT0001536 | 20005803 |
| Adipose Tissue | hsa-miR-519d-3p | MIMAT0002853 | 20057369 |
| Adipose Tissue | mmu-miR-200b-3p | MIMAT0000233 | 19787069 |
| Adipose Tissue | mmu-miR-200c-3p | MIMAT0000657 | 19787069 |
| Adipose Tissue | mmu-miR-346-5p | MIMAT0000597 | 19780716 |
| Adipose Tissue White | hsa-miR-21-5p | MIMAT0000076 | 21381024 |
| Adrenal Cortex | hsa-miR-200b-3p | MIMAT0000318 | 19849700 |
| Adrenal Glands | mmu-miR-101a-3p | MIMAT0000133 | 22128032 |
| Adrenal Glands | mmu-miR-142-3p | MIMAT0000155 | 22128032 |
| Adrenal Glands | mmu-miR-433-3p | MIMAT0001420 | 22128032 |
| Adrenal Glands | mmu-miR-96-5p | MIMAT0000541 | 22128032 |
| Adult Stem Cells | hsa-miR-34a-5p | MIMAT0000255 | 17252019 |
| Adult Stem Cells | mmu-miR-1a-3p | MIMAT0000123 | 20819939 |
| Adult Stem Cells | mmu-miR-206-3p | MIMAT0000239 | 20819939 |
| Amacrine Cells | hsa-miR-182-5p | MIMAT0000259 | 17597072 |
| Amacrine Cells | hsa-miR-96-5p | MIMAT0000095 | 17597072 |
| Amygdala | hsa-miR-183-5p | MIMAT0000261 | 19711202 |
| Amygdala | rno-miR-183-5p | MIMAT0000860 | 19711202 |
| Aorta | hsa-miR-10a-5p | MIMAT0000253 | 20624982 |
| Aorta | hsa-miR-21-5p | MIMAT0000076 | 22034194 |
| Aorta | mmu-miR-130a-3p | MIMAT0000141 | 21753805 |
| Aorta | mmu-miR-15a-5p | MIMAT0000526 | 21305018 |
| Aorta | mmu-miR-497-5p | MIMAT0003453 | 21305018 |
| Aorta Thoracic | hsa-miR-10a-5p | MIMAT0000253 | 20624982 |
| Aorta Thoracic | mmu-miR-130a-3p | MIMAT0000141 | 21753805 |
| Arteries | hsa-miR-1 | MIMAT0000416 | 21051663 |
| Arteries | hsa-miR-155-5p | MIMAT0000646 | 23108656 |
| Arteries | hsa-miR-195-5p | MIMAT0000461 | 22802111 |
| Arteries | hsa-miR-204-5p | MIMAT0000265 | 22871591 |
| Arteries | hsa-miR-21-5p | MIMAT0000076 | 22034194 |
| Arteries | hsa-miR-21-5p | MIMAT0000076 | 17478730 |
| Arteries | hsa-miR-221-3p | MIMAT0000278 | 19150885 |
| Arteries | hsa-miR-222-3p | MIMAT0000279 | 19150885 |
| Arteries | hsa-miR-31-5p | MIMAT0000089 | 23518389 |
| Arteries | hsa-miR-616-3p | MIMAT0004805 | 23497787 |
| Arteries | mmu-miR-100-3p | MIMAT0017051 | 21339483 |
| Arteries | mmu-miR-100-5p | MIMAT0000655 | 21339483 |
| Arteries | mmu-miR-130a-3p | MIMAT0000141 | 21753805 |
| Arteries | rno-miR-145-5p | MIMAT0000851 | 19542014 |
| Arteries | rno-miR-21-5p | MIMAT0000790 | 17478730 |
| Arteries | rno-miR-21-5p | MIMAT0000790 | 22565856 |
| Arteries | rno-miR-221-3p | MIMAT0000890 | 19150885 |
| Arteries | rno-miR-222-3p | MIMAT0000891 | 19150885 |
| Arteries | rno-miR-31a-5p | MIMAT0000810 | 19542014 |
| Arteries | rno-miR-31a-5p | MIMAT0000810 | 22020941 |
| Astrocytes | hsa-miR-125b-5p | MIMAT0000423 | 20347935 |
| Astrocytes | hsa-miR-326 | MIMAT0000756 | 20667897 |
| Astrocytes | hsa-miR-34a-5p | MIMAT0000255 | 19773441 |
| Astrocytes | mmu-miR-146a-5p | MIMAT0000158 | 21730286 |
| Astrocytes | mmu-miR-155-5p | MIMAT0000165 | 21247879 |
| Astrocytes | mmu-miR-298-5p | MIMAT0000376 | 21247879 |
| Astrocytes | mmu-miR-351-5p | MIMAT0000609 | 21247879 |
| Axons | rno-miR-338-3p | MIMAT0000581 | 19020050 |
| B-Lymphocytes | hsa-let-7a-5p | MIMAT0000062 | 18583325 |
| B-Lymphocytes | hsa-miR-107 | MIMAT0000104 | 19692702 |
| B-Lymphocytes | hsa-miR-125b-5p | MIMAT0000423 | 20497960 |
| B-Lymphocytes | hsa-miR-127-3p | MIMAT0000446 | 19530237 |
| B-Lymphocytes | hsa-miR-143-3p | MIMAT0000435 | 17892514 |
| B-Lymphocytes | hsa-miR-145-5p | MIMAT0000437 | 21360565 |
| B-Lymphocytes | hsa-miR-153-3p | MIMAT0000439 | 19676043 |
| B-Lymphocytes | hsa-miR-155-5p | MIMAT0000646 | 18262046 |
| B-Lymphocytes | hsa-miR-155-5p | MIMAT0000646 | 21062812 |
| B-Lymphocytes | hsa-miR-155-5p | MIMAT0000646 | 19759154 |
| B-Lymphocytes | hsa-miR-155-5p | MIMAT0000646 | 20427544 |
| B-Lymphocytes | hsa-miR-155-5p | MIMAT0000646 | 19177201 |
| B-Lymphocytes | hsa-miR-155-5p | MIMAT0000646 | 17881434 |
| B-Lymphocytes | hsa-miR-15a-5p | MIMAT0000068 | 21205967 |
| B-Lymphocytes | hsa-miR-16-5p | MIMAT0000069 | 21205967 |
| B-Lymphocytes | hsa-miR-17-5p | MIMAT0000070 | 20227518 |
| B-Lymphocytes | hsa-miR-17-5p | MIMAT0000070 | 18941111 |
| B-Lymphocytes | hsa-miR-17-5p | MIMAT0000070 | 19666108 |
| B-Lymphocytes | hsa-miR-181a-5p | MIMAT0000256 | 19692702 |
| B-Lymphocytes | hsa-miR-181a-5p | MIMAT0000256 | 20841506 |
| B-Lymphocytes | hsa-miR-181b-5p | MIMAT0000257 | 19692702 |
| B-Lymphocytes | hsa-miR-181b-5p | MIMAT0000257 | 17178851 |
| B-Lymphocytes | hsa-miR-196b-5p | MIMAT0001080 | 23293219 |
| B-Lymphocytes | hsa-miR-20a-5p | MIMAT0000075 | 19666108 |
| B-Lymphocytes | hsa-miR-20a-5p | MIMAT0000075 | 18941111 |
| B-Lymphocytes | hsa-miR-21-5p | MIMAT0000076 | 21317927 |
| B-Lymphocytes | hsa-miR-21-5p | MIMAT0000076 | 21078976 |
| B-Lymphocytes | hsa-miR-29a-3p | MIMAT0000086 | 20086245 |
| B-Lymphocytes | hsa-miR-29b-3p | MIMAT0000100 | 17178851 |
| B-Lymphocytes | hsa-miR-29b-3p | MIMAT0000100 | 20086245 |
| B-Lymphocytes | hsa-miR-29c-3p | MIMAT0000681 | 20086245 |
| B-Lymphocytes | hsa-miR-34a-5p | MIMAT0000255 | 21205967 |
| B-Lymphocytes | hsa-miR-34a-5p | MIMAT0000255 | 20598588 |
| B-Lymphocytes | hsa-miR-34b-3p | MIMAT0004676 | 21205967 |
| B-Lymphocytes | hsa-miR-34c-5p | MIMAT0000686 | 21205967 |
| B-Lymphocytes | hsa-miR-424-5p | MIMAT0001341 | 21062812 |
| B-Lymphocytes | hsa-miR-424-5p | MIMAT0001341 | 19692702 |
| B-Lymphocytes | hsa-miR-9-5p | MIMAT0000441 | 18583325 |
| B-Lymphocytes | mmu-miR-149-5p | MIMAT0000159 | 18762567 |
| B-Lymphocytes | mmu-miR-155-5p | MIMAT0000165 | 18450484 |
| B-Lymphocytes | mmu-miR-155-5p | MIMAT0000165 | 18455451 |
| B-Lymphocytes | mmu-miR-155-5p | MIMAT0000165 | 18055230 |
| B-Lymphocytes | mmu-miR-181b-5p | MIMAT0000673 | 18762567 |
| B-Lymphocytes | mmu-miR-19b-3p | MIMAT0000513 | 18762567 |
| B-Lymphocytes | mmu-miR-223-3p | MIMAT0000665 | 20935160 |
| B-Lymphocytes | mmu-miR-30b-5p | MIMAT0000130 | 18762567 |
| B-Lymphocytes | mmu-miR-34a-5p | MIMAT0000542 | 20598588 |
| Back | hsa-miR-101-3p | MIMAT0000099 | 19818710 |
| Back | hsa-miR-122-5p | MIMAT0000421 | 17179747 |
| Back | hsa-miR-199a-5p | MIMAT0000231 | 19818710 |
| Back | hsa-miR-214-3p | MIMAT0000271 | 19818710 |
| Back | hsa-miR-98-5p | MIMAT0000096 | 19818710 |
| Back | mmu-miR-122-5p | MIMAT0000246 | 17179747 |
| Back | mmu-miR-137-3p | MIMAT0000149 | 20368621 |
| Back | mmu-miR-214-3p | MIMAT0000661 | 19818710 |
| Basement Membrane | mmu-miR-200b-3p | MIMAT0000233 | 19787069 |
| Basement Membrane | mmu-miR-200c-3p | MIMAT0000657 | 19787069 |
| Bile | hsa-miR-122-5p | MIMAT0000421 | 20351063 |
| Bile | hsa-miR-15a-5p | MIMAT0000068 | 18949056 |
| Bile | hsa-miR-30a-5p | MIMAT0000087 | 19185580 |
| Bile | hsa-miR-34a-5p | MIMAT0000255 | 20185821 |
| Bile | hsa-miR-422a | MIMAT0001339 | 20351063 |
| Bile | mmu-miR-23b-3p | MIMAT0000125 | 19582816 |
| Bile | mmu-miR-24-3p | MIMAT0000219 | 19582816 |
| Bile | mmu-miR-27b-3p | MIMAT0000126 | 19582816 |
| Bile | mmu-miR-30a-5p | MIMAT0000128 | 19185580 |
| Bile | rno-miR-150-5p | MIMAT0000853 | 19892940 |
| Bile | rno-miR-194-5p | MIMAT0000869 | 19892940 |
| Bile Ducts | hsa-miR-15a-5p | MIMAT0000068 | 18949056 |
| Bile Ducts | mmu-miR-23b-3p | MIMAT0000125 | 19582816 |
| Bile Ducts | mmu-miR-24-3p | MIMAT0000219 | 19582816 |
| Bile Ducts | mmu-miR-27b-3p | MIMAT0000126 | 19582816 |
| Blastocyst | hsa-miR-19a-3p | MIMAT0000073 | 19296935 |
| Blastocyst | hsa-miR-24-3p | MIMAT0000080 | 19296935 |
| Blastocyst | mmu-miR-101a-3p | MIMAT0000133 | 17848513 |
| Blastocyst | mmu-miR-199a-3p | MIMAT0000230 | 17848513 |
| Blastocyst | mmu-miR-21a-5p | MIMAT0000530 | 18556655 |
| Blood | bta-miR-155 | MIMAT0009241 | 20735984 |
| Blood | cfa-miR-155 | MIMAT0006671 | 20735984 |
| Blood | dre-miR-155 | MIMAT0001851 | 20735984 |
| Blood | gga-miR-155 | MIMAT0001106 | 20735984 |
| Blood | hsa-let-7a-5p | MIMAT0000062 | 19939273 |
| Blood | hsa-let-7c-3p | MIMAT0026472 | 19939273 |
| Blood | hsa-let-7c-5p | MIMAT0000064 | 19939273 |
| Blood | hsa-let-7d-3p | MIMAT0004484 | 19939273 |
| Blood | hsa-let-7d-5p | MIMAT0000065 | 19939273 |
| Blood | hsa-let-7e-3p | MIMAT0004485 | 19939273 |
| Blood | hsa-let-7e-5p | MIMAT0000066 | 19939273 |
| Blood | hsa-let-7g-3p | MIMAT0004584 | 19939273 |
| Blood | hsa-let-7g-5p | MIMAT0000414 | 19939273 |
| Blood | hsa-let-7i-3p | MIMAT0004585 | 19939273 |
| Blood | hsa-let-7i-5p | MIMAT0000415 | 19939273 |
| Blood | hsa-miR-124-3p | MIMAT0000422 | 19944075 |
| Blood | hsa-miR-126-3p | MIMAT0000445 | 22867989 |
| Blood | hsa-miR-129-5p | MIMAT0000242 | 18189265 |
| Blood | hsa-miR-130b-3p | MIMAT0000691 | 18974142 |
| Blood | hsa-miR-135a-5p | MIMAT0000428 | 19944075 |
| Blood | hsa-miR-135b-5p | MIMAT0000758 | 19795981 |
| Blood | hsa-miR-146a-5p | MIMAT0000449 | 19333922 |
| Blood | hsa-miR-146a-5p | MIMAT0000449 | 20459811 |
| Blood | hsa-miR-146a-5p | MIMAT0000449 | 18759964 |
| Blood | hsa-miR-148a-3p | MIMAT0000243 | 19435428 |
| Blood | hsa-miR-155-5p | MIMAT0000646 | 20735984 |
| Blood | hsa-miR-155-5p | MIMAT0000646 | 18950466 |
| Blood | hsa-miR-155-5p | MIMAT0000646 | 17668390 |
| Blood | hsa-miR-15a-5p | MIMAT0000068 | 21205967 |
| Blood | hsa-miR-16-5p | MIMAT0000069 | 21885851 |
| Blood | hsa-miR-16-5p | MIMAT0000069 | 21205967 |
| Blood | hsa-miR-181a-5p | MIMAT0000256 | 20558617 |
| Blood | hsa-miR-181c-5p | MIMAT0000258 | 21112091 |
| Blood | hsa-miR-184 | MIMAT0000454 | 19286996 |
| Blood | hsa-miR-195-5p | MIMAT0000461 | 20952681 |
| Blood | hsa-miR-19b-3p | MIMAT0000074 | 19435428 |
| Blood | hsa-miR-20a-5p | MIMAT0000075 | 19435428 |
| Blood | hsa-miR-214-3p | MIMAT0000271 | 19435428 |
| Blood | hsa-miR-221-3p | MIMAT0000278 | 20505758 |
| Blood | hsa-miR-221-3p | MIMAT0000278 | 20516212 |
| Blood | hsa-miR-223-3p | MIMAT0000280 | 19278969 |
| Blood | hsa-miR-223-3p | MIMAT0000280 | 19017354 |
| Blood | hsa-miR-29a-3p | MIMAT0000086 | 19102781 |
| Blood | hsa-miR-29a-3p | MIMAT0000086 | 19951903 |
| Blood | hsa-miR-29b-3p | MIMAT0000100 | 19102781 |
| Blood | hsa-miR-31-3p | MIMAT0004504 | 19408243 |
| Blood | hsa-miR-31-5p | MIMAT0000089 | 19408243 |
| Blood | hsa-miR-320a | MIMAT0000510 | 18523662 |
| Blood | hsa-miR-34a-5p | MIMAT0000255 | 21205967 |
| Blood | hsa-miR-34b-3p | MIMAT0004676 | 21205967 |
| Blood | hsa-miR-34c-5p | MIMAT0000686 | 21205967 |
| Blood | hsa-miR-381-3p | MIMAT0000736 | 21435336 |
| Blood | hsa-miR-409-3p | MIMAT0001639 | 23360331 |
| Blood | hsa-miR-424-5p | MIMAT0001341 | 20972335 |
| Blood | hsa-miR-424-5p | MIMAT0001341 | 21885851 |
| Blood | hsa-miR-503-5p | MIMAT0002874 | 21220732 |
| Blood | hsa-miR-520h | MIMAT0002867 | 18189265 |
| Blood | hsa-miR-520h | MIMAT0002867 | 19435428 |
| Blood | hsa-miR-663a | MIMAT0003326 | 20622002 |
| Blood | hsa-miR-9-5p | MIMAT0000441 | 16831872 |
| Blood | hsa-miR-92a-3p | MIMAT0000092 | 19460962 |
| Blood | hsa-miR-92a-3p | MIMAT0000092 | 19435428 |
| Blood | hsa-miR-93-5p | MIMAT0000093 | 20956944 |
| Blood | hsa-miR-93-5p | MIMAT0000093 | 18974142 |
| Blood | mml-miR-155 | MIMAT0006216 | 20735984 |
| Blood | mmu-miR-100-3p | MIMAT0017051 | 21339483 |
| Blood | mmu-miR-100-5p | MIMAT0000655 | 21339483 |
| Blood | mmu-miR-125b-5p | MIMAT0000136 | 21118986 |
| Blood | mmu-miR-126a-3p | MIMAT0000138 | 18694565 |
| Blood | mmu-miR-223-3p | MIMAT0000665 | 22043014 |
| Blood | ppy-miR-155 | MIMAT0015777 | 20735984 |
| Blood | ptr-miR-155 | MIMAT0008048 | 20735984 |
| Blood | rno-let-7a-5p | MIMAT0000774 | 18258830 |
| Blood | rno-let-7b-3p | MIMAT0004705 | 18258830 |
| Blood | rno-let-7b-5p | MIMAT0000775 | 18258830 |
| Blood | rno-let-7c-5p | MIMAT0000776 | 18258830 |
| Blood | rno-let-7d-3p | MIMAT0000563 | 18258830 |
| Blood | rno-let-7d-5p | MIMAT0000562 | 18258830 |
| Blood | rno-let-7e-3p | MIMAT0004706 | 18258830 |
| Blood | rno-let-7e-5p | MIMAT0000777 | 18258830 |
| Blood | rno-let-7f-5p | MIMAT0000778 | 18258830 |
| Blood | rno-let-7i-3p | MIMAT0004707 | 18258830 |
| Blood | rno-let-7i-5p | MIMAT0000779 | 18258830 |
| Blood | rno-miR-100-5p | MIMAT0000822 | 18258830 |
| Blood | rno-miR-101a-3p | MIMAT0000823 | 18258830 |
| Blood | rno-miR-101b-3p | MIMAT0000615 | 18258830 |
| Blood | rno-miR-124-3p | MIMAT0000828 | 18258830 |
| Blood | rno-miR-125a-5p | MIMAT0000829 | 18258830 |
| Blood | rno-miR-129-5p | MIMAT0000600 | 18258830 |
| Blood | rno-miR-130a-3p | MIMAT0000836 | 18258830 |
| Blood | rno-miR-132-3p | MIMAT0000838 | 18258830 |
| Blood | rno-miR-134-5p | MIMAT0000840 | 18258830 |
| Blood | rno-miR-138-5p | MIMAT0000844 | 18258830 |
| Blood | rno-miR-139-5p | MIMAT0000845 | 18258830 |
| Blood | rno-miR-150-5p | MIMAT0000853 | 18258830 |
| Blood | rno-miR-181b-5p | MIMAT0000859 | 18258830 |
| Blood | rno-miR-185-5p | MIMAT0000862 | 18258830 |
| Blood | rno-miR-204-5p | MIMAT0000877 | 18258830 |
| Blood | rno-miR-21-5p | MIMAT0000790 | 18258830 |
| Blood | rno-miR-214-3p | MIMAT0000885 | 18258830 |
| Blood | rno-miR-215 | MIMAT0003118 | 18258830 |
| Blood | rno-miR-218a-5p | MIMAT0000888 | 18258830 |
| Blood | rno-miR-223-3p | MIMAT0000892 | 18258830 |
| Blood | rno-miR-23a-3p | MIMAT0000792 | 18258830 |
| Blood | rno-miR-23b-3p | MIMAT0000793 | 18258830 |
| Blood | rno-miR-24-3p | MIMAT0000794 | 18258830 |
| Blood | rno-miR-25-3p | MIMAT0000795 | 18258830 |
| Blood | rno-miR-26b-5p | MIMAT0000797 | 18258830 |
| Blood | rno-miR-27a-3p | MIMAT0000799 | 18258830 |
| Blood | rno-miR-290 | MIMAT0000893 | 18258830 |
| Blood | rno-miR-292-5p | MIMAT0000896 | 18258830 |
| Blood | rno-miR-293-5p | MIMAT0012847 | 18258830 |
| Blood | rno-miR-298-5p | MIMAT0000900 | 18258830 |
| Blood | rno-miR-29a-3p | MIMAT0000802 | 18258830 |
| Blood | rno-miR-29b-3p | MIMAT0000801 | 18258830 |
| Blood | rno-miR-29c-3p | MIMAT0000803 | 18258830 |
| Blood | rno-miR-301a-3p | MIMAT0000552 | 18258830 |
| Blood | rno-miR-30a-3p | MIMAT0000809 | 18258830 |
| Blood | rno-miR-30a-5p | MIMAT0000808 | 18258830 |
| Blood | rno-miR-30b-5p | MIMAT0000806 | 18258830 |
| Blood | rno-miR-30c-5p | MIMAT0000804 | 18258830 |
| Blood | rno-miR-30e-5p | MIMAT0000805 | 18258830 |
| Blood | rno-miR-320-3p | MIMAT0000903 | 18258830 |
| Blood | rno-miR-322-5p | MIMAT0001619 | 18258830 |
| Blood | rno-miR-324-5p | MIMAT0000553 | 18258830 |
| Blood | rno-miR-327 | MIMAT0000561 | 18258830 |
| Blood | rno-miR-328a-3p | MIMAT0000564 | 18258830 |
| Blood | rno-miR-331-3p | MIMAT0000570 | 18258830 |
| Blood | rno-miR-338-3p | MIMAT0000581 | 18258830 |
| Blood | rno-miR-342-3p | MIMAT0000589 | 18258830 |
| Blood | rno-miR-342-5p | MIMAT0004652 | 18258830 |
| Blood | rno-miR-34a-5p | MIMAT0000815 | 18258830 |
| Blood | rno-miR-374-5p | MIMAT0003208 | 18258830 |
| Blood | rno-miR-382-5p | MIMAT0003201 | 18258830 |
| Blood | rno-miR-383-5p | MIMAT0003114 | 18258830 |
| Blood | rno-miR-409a-3p | MIMAT0003205 | 18258830 |
| Blood | rno-miR-485-5p | MIMAT0003203 | 18258830 |
| Blood | rno-miR-494-3p | MIMAT0003193 | 18258830 |
| Blood | rno-miR-497-5p | MIMAT0003383 | 18258830 |
| Blood | rno-miR-543-5p | MIMAT0004787 | 18258830 |
| Blood | rno-miR-664-3p | MIMAT0003382 | 18258830 |
| Blood | rno-miR-7a-5p | MIMAT0000606 | 18258830 |
| Blood | rno-miR-92a-3p | MIMAT0000816 | 18258830 |
| Blood | rno-miR-98-5p | MIMAT0000819 | 18258830 |
| Blood | rno-miR-99a-5p | MIMAT0000820 | 18258830 |
| Blood | rno-miR-99b-5p | MIMAT0000821 | 18258830 |
| Blood | rno-miR-9a-5p | MIMAT0000781 | 18258830 |
| Blood | rno-miR-9a-5p | MIMAT0000781 | 16831872 |
| Blood | xtr-miR-155 | MIMAT0003608 | 20735984 |
| Blood Cells | hsa-miR-148a-3p | MIMAT0000243 | 19435428 |
| Blood Cells | hsa-miR-19b-3p | MIMAT0000074 | 19435428 |
| Blood Cells | hsa-miR-20a-5p | MIMAT0000075 | 19435428 |
| Blood Cells | hsa-miR-214-3p | MIMAT0000271 | 19435428 |
| Blood Cells | hsa-miR-520h | MIMAT0002867 | 19435428 |
| Blood Cells | hsa-miR-92a-3p | MIMAT0000092 | 19435428 |
| Blood Cells | mmu-miR-125b-5p | MIMAT0000136 | 21118986 |
| Blood Platelets | hsa-let-7a-5p | MIMAT0000062 | 19939273 |
| Blood Platelets | hsa-let-7a-5p | MIMAT0000062 | 20445018 |
| Blood Platelets | hsa-let-7c-3p | MIMAT0026472 | 19939273 |
| Blood Platelets | hsa-let-7c-5p | MIMAT0000064 | 20445018 |
| Blood Platelets | hsa-let-7c-5p | MIMAT0000064 | 19939273 |
| Blood Platelets | hsa-let-7d-3p | MIMAT0004484 | 19939273 |
| Blood Platelets | hsa-let-7d-5p | MIMAT0000065 | 20445018 |
| Blood Platelets | hsa-let-7d-5p | MIMAT0000065 | 21266476 |
| Blood Platelets | hsa-let-7d-5p | MIMAT0000065 | 19939273 |
| Blood Platelets | hsa-let-7e-3p | MIMAT0004485 | 19939273 |
| Blood Platelets | hsa-let-7e-5p | MIMAT0000066 | 19939273 |
| Blood Platelets | hsa-let-7e-5p | MIMAT0000066 | 20445018 |
| Blood Platelets | hsa-let-7f-5p | MIMAT0000067 | 20445018 |
| Blood Platelets | hsa-let-7g-3p | MIMAT0004584 | 19939273 |
| Blood Platelets | hsa-let-7g-5p | MIMAT0000414 | 19939273 |
| Blood Platelets | hsa-let-7i-3p | MIMAT0004585 | 19939273 |
| Blood Platelets | hsa-let-7i-5p | MIMAT0000415 | 19939273 |
| Blood Platelets | hsa-miR-10a-5p | MIMAT0000253 | 16549775 |
| Blood Platelets | hsa-miR-130a-3p | MIMAT0000425 | 16549775 |
| Blood Platelets | hsa-miR-146b-3p | MIMAT0004766 | 21266476 |
| Blood Platelets | hsa-miR-151a-5p | MIMAT0004697 | 20445018 |
| Blood Platelets | hsa-miR-28-5p | MIMAT0000085 | 20445018 |
| Blood Platelets | hsa-miR-520a-3p | MIMAT0002834 | 23389994 |
| Blood Platelets | hsa-miR-520b | MIMAT0002843 | 23389994 |
| Blood Platelets | hsa-miR-520e | MIMAT0002825 | 23389994 |
| Blood Platelets | hsa-miR-708-3p | MIMAT0004927 | 20445018 |
| Blood Platelets | hsa-miR-98-5p | MIMAT0000096 | 20445018 |
| Blood Vessels | hsa-miR-126-3p | MIMAT0000445 | 22867989 |
| Blood Vessels | hsa-miR-16-5p | MIMAT0000069 | 21885851 |
| Blood Vessels | hsa-miR-221-3p | MIMAT0000278 | 20516212 |
| Blood Vessels | hsa-miR-424-5p | MIMAT0001341 | 21885851 |
| Blood Vessels | hsa-miR-92a-3p | MIMAT0000092 | 19460962 |
| Blood Vessels | hsa-miR-93-5p | MIMAT0000093 | 20956944 |
| Blood Vessels | mmu-miR-100-3p | MIMAT0017051 | 21339483 |
| Blood Vessels | mmu-miR-100-5p | MIMAT0000655 | 21339483 |
| Blood Vessels | mmu-miR-126a-3p | MIMAT0000138 | 18694565 |
| Blood-Brain Barrier | mmu-miR-15a-5p | MIMAT0000526 | 20445066 |
| Bodily Secretions | hsa-let-7d-5p | MIMAT0000065 | 21616524 |
| Bodily Secretions | hsa-let-7f-5p | MIMAT0000067 | 21616524 |
| Bodily Secretions | hsa-let-7g-5p | MIMAT0000414 | 21616524 |
| Bodily Secretions | hsa-let-7i-5p | MIMAT0000415 | 21616524 |
| Bodily Secretions | hsa-miR-107 | MIMAT0000104 | 20489155 |
| Bodily Secretions | hsa-miR-124-3p | MIMAT0000422 | 19404929 |
| Bodily Secretions | hsa-miR-125a-5p | MIMAT0000443 | 19011087 |
| Bodily Secretions | hsa-miR-125b-5p | MIMAT0000423 | 19011087 |
| Bodily Secretions | hsa-miR-126-3p | MIMAT0000445 | 20083669 |
| Bodily Secretions | hsa-miR-146a-5p | MIMAT0000449 | 21236257 |
| Bodily Secretions | hsa-miR-146a-5p | MIMAT0000449 | 20181935 |
| Bodily Secretions | hsa-miR-15a-5p | MIMAT0000068 | 19144710 |
| Bodily Secretions | hsa-miR-16-5p | MIMAT0000069 | 19144710 |
| Bodily Secretions | hsa-miR-17-5p | MIMAT0000070 | 20406904 |
| Bodily Secretions | hsa-miR-181a-5p | MIMAT0000256 | 22956783 |
| Bodily Secretions | hsa-miR-184 | MIMAT0000454 | 19286996 |
| Bodily Secretions | hsa-miR-199a-5p | MIMAT0000231 | 19011087 |
| Bodily Secretions | hsa-miR-19a-3p | MIMAT0000073 | 22105995 |
| Bodily Secretions | hsa-miR-19b-3p | MIMAT0000074 | 22105995 |
| Bodily Secretions | hsa-miR-20a-5p | MIMAT0000075 | 20406904 |
| Bodily Secretions | hsa-miR-21-5p | MIMAT0000076 | 21469086 |
| Bodily Secretions | hsa-miR-214-3p | MIMAT0000271 | 22227154 |
| Bodily Secretions | hsa-miR-26a-5p | MIMAT0000082 | 19011087 |
| Bodily Secretions | hsa-miR-29a-3p | MIMAT0000086 | 21276447 |
| Bodily Secretions | hsa-miR-29a-3p | MIMAT0000086 | 21665270 |
| Bodily Secretions | hsa-miR-29b-3p | MIMAT0000100 | 21665270 |
| Bodily Secretions | hsa-miR-29b-3p | MIMAT0000100 | 20479936 |
| Bodily Secretions | hsa-miR-30a-5p | MIMAT0000087 | 23338554 |
| Bodily Secretions | hsa-miR-33a-5p | MIMAT0000091 | 23453925 |
| Bodily Secretions | hsa-miR-346 | MIMAT0000773 | 19011087 |
| Bodily Secretions | hsa-miR-375 | MIMAT0000728 | 15538371 |
| Bodily Secretions | hsa-miR-378a-3p | MIMAT0000732 | 19844573 |
| Bodily Secretions | hsa-miR-9-5p | MIMAT0000441 | 19008124 |
| Bodily Secretions | hsa-miR-9-5p | MIMAT0000441 | 19572217 |
| Bodily Secretions | hsa-miR-9-5p | MIMAT0000441 | 20479936 |
| Bodily Secretions | hsa-miR-98-5p | MIMAT0000096 | 21616524 |
| Bodily Secretions | mmu-miR-124-3p | MIMAT0000134 | 17462994 |
| Bodily Secretions | mmu-miR-133a-3p | MIMAT0000145 | 19043405 |
| Bodily Secretions | mmu-miR-206-3p | MIMAT0000239 | 19043405 |
| Bodily Secretions | mmu-miR-21a-5p | MIMAT0000530 | 19043405 |
| Bodily Secretions | mmu-miR-29a-3p | MIMAT0000535 | 21665270 |
| Bodily Secretions | mmu-miR-29b-3p | MIMAT0000127 | 21665270 |
| Bodily Secretions | mmu-miR-375-3p | MIMAT0000739 | 20224724 |
| Bodily Secretions | mmu-miR-375-3p | MIMAT0000739 | 15538371 |
| Bodily Secretions | mmu-miR-9-5p | MIMAT0000142 | 21288303 |
| Bodily Secretions | mmu-miR-93-5p | MIMAT0000540 | 20501654 |
| Bodily Secretions | rno-miR-150-5p | MIMAT0000853 | 19892940 |
| Bodily Secretions | rno-miR-194-5p | MIMAT0000869 | 19892940 |
| Bone and Bones | bta-miR-130a | MIMAT0009223 | 17855557 |
| Bone and Bones | bta-miR-335 | MIMAT0009291 | 18185580 |
| Bone and Bones | cfa-miR-130a | MIMAT0006631 | 17855557 |
| Bone and Bones | cfa-miR-335 | MIMAT0006624 | 18185580 |
| Bone and Bones | dre-miR-130a | MIMAT0001826 | 17855557 |
| Bone and Bones | hsa-miR-100-5p | MIMAT0000098 | 22684006 |
| Bone and Bones | hsa-miR-106b-5p | MIMAT0000680 | 18728182 |
| Bone and Bones | hsa-miR-125a-5p | MIMAT0000443 | 19011087 |
| Bone and Bones | hsa-miR-125b-5p | MIMAT0000423 | 19011087 |
| Bone and Bones | hsa-miR-125b-5p | MIMAT0000423 | 19738052 |
| Bone and Bones | hsa-miR-126-3p | MIMAT0000445 | 18474618 |
| Bone and Bones | hsa-miR-126-3p | MIMAT0000445 | 18832181 |
| Bone and Bones | hsa-miR-130a-3p | MIMAT0000425 | 17855557 |
| Bone and Bones | hsa-miR-133a-3p | MIMAT0000427 | 23069713 |
| Bone and Bones | hsa-miR-135b-5p | MIMAT0000758 | 19795981 |
| Bone and Bones | hsa-miR-140-5p | MIMAT0000431 | 21576357 |
| Bone and Bones | hsa-miR-141-3p | MIMAT0000432 | 19454767 |
| Bone and Bones | hsa-miR-142-3p | MIMAT0000434 | 21394831 |
| Bone and Bones | hsa-miR-145-5p | MIMAT0000437 | 23355420 |
| Bone and Bones | hsa-miR-145-5p | MIMAT0000437 | 18474618 |
| Bone and Bones | hsa-miR-146a-5p | MIMAT0000449 | 18174313 |
| Bone and Bones | hsa-miR-148a-3p | MIMAT0000243 | 19435428 |
| Bone and Bones | hsa-miR-155-5p | MIMAT0000646 | 18299402 |
| Bone and Bones | hsa-miR-155-5p | MIMAT0000646 | 20427544 |
| Bone and Bones | hsa-miR-155-5p | MIMAT0000646 | 20133617 |
| Bone and Bones | hsa-miR-16-5p | MIMAT0000069 | 19738602 |
| Bone and Bones | hsa-miR-17-5p | MIMAT0000070 | 19390056 |
| Bone and Bones | hsa-miR-181a-5p | MIMAT0000256 | 22956783 |
| Bone and Bones | hsa-miR-181a-5p | MIMAT0000256 | 18728182 |
| Bone and Bones | hsa-miR-181b-5p | MIMAT0000257 | 18728182 |
| Bone and Bones | hsa-miR-196a-5p | MIMAT0000226 | 20480203 |
| Bone and Bones | hsa-miR-199a-5p | MIMAT0000231 | 22441842 |
| Bone and Bones | hsa-miR-199a-5p | MIMAT0000231 | 19011087 |
| Bone and Bones | hsa-miR-19a-3p | MIMAT0000073 | 18728182 |
| Bone and Bones | hsa-miR-19a-3p | MIMAT0000073 | 19390056 |
| Bone and Bones | hsa-miR-19b-3p | MIMAT0000074 | 19435428 |
| Bone and Bones | hsa-miR-19b-3p | MIMAT0000074 | 19390056 |
| Bone and Bones | hsa-miR-19b-3p | MIMAT0000074 | 18728182 |
| Bone and Bones | hsa-miR-200a-3p | MIMAT0000682 | 19454767 |
| Bone and Bones | hsa-miR-206 | MIMAT0000462 | 17855557 |
| Bone and Bones | hsa-miR-20a-5p | MIMAT0000075 | 19435428 |
| Bone and Bones | hsa-miR-20a-5p | MIMAT0000075 | 19390056 |
| Bone and Bones | hsa-miR-21-5p | MIMAT0000076 | 19578724 |
| Bone and Bones | hsa-miR-214-3p | MIMAT0000271 | 19435428 |
| Bone and Bones | hsa-miR-24-3p | MIMAT0000080 | 20019669 |
| Bone and Bones | hsa-miR-25-3p | MIMAT0000081 | 18728182 |
| Bone and Bones | hsa-miR-26a-5p | MIMAT0000082 | 18197755 |
| Bone and Bones | hsa-miR-26a-5p | MIMAT0000082 | 19011087 |
| Bone and Bones | hsa-miR-27a-3p | MIMAT0000084 | 19440384 |
| Bone and Bones | hsa-miR-302a-3p | MIMAT0000684 | 17855557 |
| Bone and Bones | hsa-miR-30a-5p | MIMAT0000087 | 23287430 |
| Bone and Bones | hsa-miR-30c-5p | MIMAT0000244 | 23469762 |
| Bone and Bones | hsa-miR-32-5p | MIMAT0000090 | 18728182 |
| Bone and Bones | hsa-miR-320a | MIMAT0000510 | 17855557 |
| Bone and Bones | hsa-miR-335-5p | MIMAT0000765 | 18185580 |
| Bone and Bones | hsa-miR-346 | MIMAT0000773 | 19011087 |
| Bone and Bones | hsa-miR-34a-5p | MIMAT0000255 | 20598588 |
| Bone and Bones | hsa-miR-34b-3p | MIMAT0004676 | 22498974 |
| Bone and Bones | hsa-miR-34c-5p | MIMAT0000686 | 22498974 |
| Bone and Bones | hsa-miR-378a-3p | MIMAT0000732 | 19844573 |
| Bone and Bones | hsa-miR-449a | MIMAT0001541 | 21569010 |
| Bone and Bones | hsa-miR-489-3p | MIMAT0002805 | 19440384 |
| Bone and Bones | hsa-miR-520h | MIMAT0002867 | 19435428 |
| Bone and Bones | hsa-miR-637 | MIMAT0003307 | 21880893 |
| Bone and Bones | hsa-miR-9-5p | MIMAT0000441 | 19008124 |
| Bone and Bones | hsa-miR-92a-3p | MIMAT0000092 | 19435428 |
| Bone and Bones | hsa-miR-92a-3p | MIMAT0000092 | 18728182 |
| Bone and Bones | hsa-miR-92a-3p | MIMAT0000092 | 19390056 |
| Bone and Bones | hsa-miR-93-5p | MIMAT0000093 | 18728182 |
| Bone and Bones | mmu-let-7a-5p | MIMAT0000521 | 18474618 |
| Bone and Bones | mmu-miR-122-5p | MIMAT0000246 | 16258535 |
| Bone and Bones | mmu-miR-122-5p | MIMAT0000246 | 21364282 |
| Bone and Bones | mmu-miR-126a-3p | MIMAT0000138 | 18474618 |
| Bone and Bones | mmu-miR-133a-3p | MIMAT0000145 | 18784367 |
| Bone and Bones | mmu-miR-135a-5p | MIMAT0000147 | 18784367 |
| Bone and Bones | mmu-miR-140-5p | MIMAT0000151 | 16828749 |
| Bone and Bones | mmu-miR-140-5p | MIMAT0000151 | 21576357 |
| Bone and Bones | mmu-miR-141-3p | MIMAT0000153 | 19454767 |
| Bone and Bones | mmu-miR-145a-5p | MIMAT0000157 | 18474618 |
| Bone and Bones | mmu-miR-199a-3p | MIMAT0000230 | 19251704 |
| Bone and Bones | mmu-miR-200a-3p | MIMAT0000519 | 19454767 |
| Bone and Bones | mmu-miR-204-5p | MIMAT0000237 | 20039258 |
| Bone and Bones | mmu-miR-206-3p | MIMAT0000239 | 19933329 |
| Bone and Bones | mmu-miR-211-5p | MIMAT0000668 | 20039258 |
| Bone and Bones | mmu-miR-21a-5p | MIMAT0000530 | 21273303 |
| Bone and Bones | mmu-miR-23b-3p | MIMAT0000125 | 19582816 |
| Bone and Bones | mmu-miR-24-3p | MIMAT0000219 | 19582816 |
| Bone and Bones | mmu-miR-27b-3p | MIMAT0000126 | 19342382 |
| Bone and Bones | mmu-miR-27b-3p | MIMAT0000126 | 19582816 |
| Bone and Bones | mmu-miR-2861 | MIMAT0013803 | 19920351 |
| Bone and Bones | mmu-miR-29b-3p | MIMAT0000127 | 19342382 |
| Bone and Bones | mmu-miR-34a-5p | MIMAT0000542 | 20424141 |
| Bone and Bones | mmu-miR-34a-5p | MIMAT0000542 | 20598588 |
| Bone and Bones | mmu-miR-34c-5p | MIMAT0000381 | 18784367 |
| Bone and Bones | mmu-miR-370-3p | MIMAT0001095 | 22587915 |
| Bone and Bones | mmu-miR-3960 | MIMAT0019336 | 21324897 |
| Bone and Bones | mmu-miR-9-5p | MIMAT0000142 | 22465325 |
| Bone and Bones | ppy-miR-130a | MIMAT0015753 | 17855557 |
| Bone and Bones | ppy-miR-335 | MIMAT0015837 | 18185580 |
| Bone and Bones | ptr-miR-130a | MIMAT0008025 | 17855557 |
| Bone and Bones | ptr-miR-335 | MIMAT0008104 | 18185580 |
| Bone and Bones | rno-miR-335 | MIMAT0000575 | 18185580 |
| Bone and Bones | ssc-miR-130a | MIMAT0007758 | 17855557 |
| Bone and Bones | ssc-miR-335 | MIMAT0013955 | 18185580 |
| Bone and Bones | xtr-miR-130a | MIMAT0003591 | 17855557 |
| Bone Marrow | hsa-miR-125a-5p | MIMAT0000443 | 19011087 |
| Bone Marrow | hsa-miR-125b-5p | MIMAT0000423 | 19011087 |
| Bone Marrow | hsa-miR-126-3p | MIMAT0000445 | 18474618 |
| Bone Marrow | hsa-miR-145-5p | MIMAT0000437 | 18474618 |
| Bone Marrow | hsa-miR-155-5p | MIMAT0000646 | 18299402 |
| Bone Marrow | hsa-miR-199a-5p | MIMAT0000231 | 19011087 |
| Bone Marrow | hsa-miR-203a | MIMAT0000264 | 21707582 |
| Bone Marrow | hsa-miR-26a-5p | MIMAT0000082 | 19011087 |
| Bone Marrow | hsa-miR-346 | MIMAT0000773 | 19011087 |
| Bone Marrow | hsa-miR-637 | MIMAT0003307 | 21880893 |
| Bone Marrow | mmu-let-7a-5p | MIMAT0000521 | 18474618 |
| Bone Marrow | mmu-miR-126a-3p | MIMAT0000138 | 18474618 |
| Bone Marrow | mmu-miR-145a-5p | MIMAT0000157 | 18474618 |
| Bone Marrow | mmu-miR-9-5p | MIMAT0000142 | 22465325 |
| Bone Marrow Cells | hsa-miR-126-3p | MIMAT0000445 | 18474618 |
| Bone Marrow Cells | hsa-miR-145-5p | MIMAT0000437 | 18474618 |
| Bone Marrow Cells | mmu-let-7a-5p | MIMAT0000521 | 18474618 |
| Bone Marrow Cells | mmu-miR-126a-3p | MIMAT0000138 | 18474618 |
| Bone Marrow Cells | mmu-miR-145a-5p | MIMAT0000157 | 18474618 |
| Brain | hsa-let-7a-5p | MIMAT0000062 | 20668208 |
| Brain | hsa-let-7a-5p | MIMAT0000062 | 19110058 |
| Brain | hsa-let-7b-5p | MIMAT0000063 | 20133835 |
| Brain | hsa-let-7d-3p | MIMAT0004484 | 19703567 |
| Brain | hsa-let-7d-5p | MIMAT0000065 | 21266476 |
| Brain | hsa-let-7d-5p | MIMAT0000065 | 19703567 |
| Brain | hsa-let-7d-5p | MIMAT0000065 | 19110058 |
| Brain | hsa-miR-1 | MIMAT0000416 | 15685193 |
| Brain | hsa-miR-106a-5p | MIMAT0000103 | 19110058 |
| Brain | hsa-miR-106b-5p | MIMAT0000680 | 19110058 |
| Brain | hsa-miR-107 | MIMAT0000104 | 18234899 |
| Brain | hsa-miR-107 | MIMAT0000104 | 20489155 |
| Brain | hsa-miR-10a-5p | MIMAT0000253 | 20506192 |
| Brain | hsa-miR-124-3p | MIMAT0000422 | 20362537 |
| Brain | hsa-miR-124-3p | MIMAT0000422 | 18434550 |
| Brain | hsa-miR-124-3p | MIMAT0000422 | 18577219 |
| Brain | hsa-miR-124-3p | MIMAT0000422 | 21196113 |
| Brain | hsa-miR-124-3p | MIMAT0000422 | 18607543 |
| Brain | hsa-miR-124-3p | MIMAT0000422 | 19703567 |
| Brain | hsa-miR-124-3p | MIMAT0000422 | 15685193 |
| Brain | hsa-miR-124-3p | MIMAT0000422 | 19131573 |
| Brain | hsa-miR-1258 | MIMAT0005909 | 21266359 |
| Brain | hsa-miR-125a-5p | MIMAT0000443 | 18973228 |
| Brain | hsa-miR-125b-5p | MIMAT0000423 | 20347935 |
| Brain | hsa-miR-125b-5p | MIMAT0000423 | 19293287 |
| Brain | hsa-miR-125b-5p | MIMAT0000423 | 20159450 |
| Brain | hsa-miR-128-3p | MIMAT0000424 | 18810376 |
| Brain | hsa-miR-128-3p | MIMAT0000424 | 19010882 |
| Brain | hsa-miR-128-3p | MIMAT0000424 | 19941032 |
| Brain | hsa-miR-128-3p | MIMAT0000424 | 21143953 |
| Brain | hsa-miR-130a-3p | MIMAT0000425 | 19110058 |
| Brain | hsa-miR-132-3p | MIMAT0000426 | 20010955 |
| Brain | hsa-miR-132-3p | MIMAT0000426 | 18082412 |
| Brain | hsa-miR-137 | MIMAT0000429 | 23252729 |
| Brain | hsa-miR-137 | MIMAT0000429 | 18577219 |
| Brain | hsa-miR-146a-5p | MIMAT0000449 | 20181935 |
| Brain | hsa-miR-146a-5p | MIMAT0000449 | 18801740 |
| Brain | hsa-miR-146a-5p | MIMAT0000449 | 19540598 |
| Brain | hsa-miR-146b-3p | MIMAT0004766 | 21266476 |
| Brain | hsa-miR-151a-3p | MIMAT0000757 | 21143953 |
| Brain | hsa-miR-153-3p | MIMAT0000439 | 19676043 |
| Brain | hsa-miR-155-5p | MIMAT0000646 | 19897480 |
| Brain | hsa-miR-15a-5p | MIMAT0000068 | 19110058 |
| Brain | hsa-miR-15a-5p | MIMAT0000068 | 18434550 |
| Brain | hsa-miR-15b-5p | MIMAT0000417 | 19135980 |
| Brain | hsa-miR-16-1-3p | MIMAT0004489 | 23175429 |
| Brain | hsa-miR-17-5p | MIMAT0000070 | 19110058 |
| Brain | hsa-miR-182-5p | MIMAT0000259 | 17597072 |
| Brain | hsa-miR-183-5p | MIMAT0000261 | 19711202 |
| Brain | hsa-miR-185-5p | MIMAT0000455 | 21143953 |
| Brain | hsa-miR-18a-5p | MIMAT0000072 | 19131573 |
| Brain | hsa-miR-195-5p | MIMAT0000461 | 22217655 |
| Brain | hsa-miR-199b-5p | MIMAT0000263 | 19308264 |
| Brain | hsa-miR-19b-3p | MIMAT0000074 | 18434550 |
| Brain | hsa-miR-200a-3p | MIMAT0000682 | 19703993 |
| Brain | hsa-miR-200b-3p | MIMAT0000318 | 17923093 |
| Brain | hsa-miR-205-5p | MIMAT0000266 | 23125283 |
| Brain | hsa-miR-20a-5p | MIMAT0000075 | 19110058 |
| Brain | hsa-miR-21-5p | MIMAT0000076 | 18372920 |
| Brain | hsa-miR-21-5p | MIMAT0000076 | 21170291 |
| Brain | hsa-miR-219a-5p | MIMAT0000276 | 19196972 |
| Brain | hsa-miR-221-3p | MIMAT0000278 | 18759060 |
| Brain | hsa-miR-221-3p | MIMAT0000278 | 17721077 |
| Brain | hsa-miR-222-3p | MIMAT0000279 | 17721077 |
| Brain | hsa-miR-24-1-5p | MIMAT0000079 | 16224024 |
| Brain | hsa-miR-26a-5p | MIMAT0000082 | 19487573 |
| Brain | hsa-miR-26a-5p | MIMAT0000082 | 20080666 |
| Brain | hsa-miR-296-5p | MIMAT0000690 | 18977327 |
| Brain | hsa-miR-29a-3p | MIMAT0000086 | 19584290 |
| Brain | hsa-miR-29a-3p | MIMAT0000086 | 20202123 |
| Brain | hsa-miR-29a-3p | MIMAT0000086 | 18434550 |
| Brain | hsa-miR-29b-3p | MIMAT0000100 | 18434550 |
| Brain | hsa-miR-29c-3p | MIMAT0000681 | 21565331 |
| Brain | hsa-miR-320a | MIMAT0000510 | 20628061 |
| Brain | hsa-miR-326 | MIMAT0000756 | 19955368 |
| Brain | hsa-miR-326 | MIMAT0000756 | 20667897 |
| Brain | hsa-miR-34a-5p | MIMAT0000255 | 20470934 |
| Brain | hsa-miR-34a-5p | MIMAT0000255 | 19773441 |
| Brain | hsa-miR-373-3p | MIMAT0000726 | 15685193 |
| Brain | hsa-miR-383-5p | MIMAT0000738 | 23227829 |
| Brain | hsa-miR-383-5p | MIMAT0000738 | 23564324 |
| Brain | hsa-miR-429 | MIMAT0001536 | 17923093 |
| Brain | hsa-miR-433-3p | MIMAT0001627 | 18252210 |
| Brain | hsa-miR-483-5p | MIMAT0004761 | 22465663 |
| Brain | hsa-miR-509-3p | MIMAT0002881 | 21143953 |
| Brain | hsa-miR-7-5p | MIMAT0000252 | 18483236 |
| Brain | hsa-miR-7-5p | MIMAT0000252 | 19628698 |
| Brain | hsa-miR-758-3p | MIMAT0003879 | 21885853 |
| Brain | hsa-miR-802 | MIMAT0004185 | 19897480 |
| Brain | hsa-miR-9-3p | MIMAT0000442 | 19118166 |
| Brain | hsa-miR-9-5p | MIMAT0000441 | 19330006 |
| Brain | hsa-miR-9-5p | MIMAT0000441 | 18434550 |
| Brain | hsa-miR-9-5p | MIMAT0000441 | 19118166 |
| Brain | hsa-miR-9-5p | MIMAT0000441 | 18973228 |
| Brain | hsa-miR-9-5p | MIMAT0000441 | 20362537 |
| Brain | hsa-miR-96-5p | MIMAT0000095 | 17597072 |
| Brain | mmu-let-7b-5p | MIMAT0000522 | 19536157 |
| Brain | mmu-let-7b-5p | MIMAT0000522 | 20133835 |
| Brain | mmu-let-7d-5p | MIMAT0000383 | 19703567 |
| Brain | mmu-miR-124-3p | MIMAT0000134 | 18577219 |
| Brain | mmu-miR-124-3p | MIMAT0000134 | 19287386 |
| Brain | mmu-miR-124-3p | MIMAT0000134 | 19703567 |
| Brain | mmu-miR-134-5p | MIMAT0000146 | 20622856 |
| Brain | mmu-miR-134-5p | MIMAT0000146 | 20126250 |
| Brain | mmu-miR-136-5p | MIMAT0000148 | 19536157 |
| Brain | mmu-miR-137-3p | MIMAT0000149 | 18577219 |
| Brain | mmu-miR-137-5p | MIMAT0016986 | 18577219 |
| Brain | mmu-miR-146a-5p | MIMAT0000158 | 21730286 |
| Brain | mmu-miR-149-5p | MIMAT0000159 | 19536157 |
| Brain | mmu-miR-155-5p | MIMAT0000165 | 21247879 |
| Brain | mmu-miR-155-5p | MIMAT0000165 | 22043967 |
| Brain | mmu-miR-15a-5p | MIMAT0000526 | 19536157 |
| Brain | mmu-miR-17-5p | MIMAT0000649 | 19536157 |
| Brain | mmu-miR-181a-5p | MIMAT0000210 | 19536157 |
| Brain | mmu-miR-190a-5p | MIMAT0000220 | 19536157 |
| Brain | mmu-miR-19b-3p | MIMAT0000513 | 19536157 |
| Brain | mmu-miR-1a-3p | MIMAT0000123 | 17234972 |
| Brain | mmu-miR-205-5p | MIMAT0000238 | 23125283 |
| Brain | mmu-miR-219a-5p | MIMAT0000664 | 19196972 |
| Brain | mmu-miR-21a-5p | MIMAT0000530 | 18372920 |
| Brain | mmu-miR-223-3p | MIMAT0000665 | 19536157 |
| Brain | mmu-miR-23a-3p | MIMAT0000532 | 19259393 |
| Brain | mmu-miR-23b-3p | MIMAT0000125 | 18716031 |
| Brain | mmu-miR-23b-3p | MIMAT0000125 | 19259393 |
| Brain | mmu-miR-24-3p | MIMAT0000219 | 19536157 |
| Brain | mmu-miR-26a-5p | MIMAT0000533 | 19536157 |
| Brain | mmu-miR-297a-5p | MIMAT0000375 | 19536157 |
| Brain | mmu-miR-298-5p | MIMAT0000376 | 18986979 |
| Brain | mmu-miR-298-5p | MIMAT0000376 | 21247879 |
| Brain | mmu-miR-301b-3p | MIMAT0004186 | 19536157 |
| Brain | mmu-miR-30a-5p | MIMAT0000128 | 20921386 |
| Brain | mmu-miR-30d-5p | MIMAT0000515 | 20921386 |
| Brain | mmu-miR-30e-5p | MIMAT0000248 | 19536157 |
| Brain | mmu-miR-324-3p | MIMAT0000556 | 19536157 |
| Brain | mmu-miR-328-3p | MIMAT0000565 | 18986979 |
| Brain | mmu-miR-340-5p | MIMAT0004651 | 19536157 |
| Brain | mmu-miR-346-5p | MIMAT0000597 | 19780716 |
| Brain | mmu-miR-34b-5p | MIMAT0000382 | 19536157 |
| Brain | mmu-miR-351-5p | MIMAT0000609 | 21247879 |
| Brain | mmu-miR-362-5p | MIMAT0000706 | 19536157 |
| Brain | mmu-miR-381-3p | MIMAT0000746 | 20921386 |
| Brain | mmu-miR-425-5p | MIMAT0004750 | 19536157 |
| Brain | mmu-miR-449a-5p | MIMAT0001542 | 19056356 |
| Brain | mmu-miR-451a | MIMAT0001632 | 19536157 |
| Brain | mmu-miR-495-3p | MIMAT0003456 | 20921386 |
| Brain | mmu-miR-497-5p | MIMAT0003453 | 20053374 |
| Brain | mmu-miR-541-5p | MIMAT0003170 | 19536157 |
| Brain | mmu-miR-743a-3p | MIMAT0004238 | 21623795 |
| Brain | mmu-miR-758-3p | MIMAT0003889 | 19536157 |
| Brain | mmu-miR-7b-5p | MIMAT0000678 | 19536157 |
| Brain | mmu-miR-7b-5p | MIMAT0000678 | 17028171 |
| Brain | mmu-miR-9-5p | MIMAT0000142 | 19536157 |
| Brain | mmu-miR-9-5p | MIMAT0000142 | 20362537 |
| Brain | mmu-miR-9-5p | MIMAT0000142 | 18842901 |
| Brain | rno-let-7a-5p | MIMAT0000774 | 18258830 |
| Brain | rno-let-7b-3p | MIMAT0004705 | 18258830 |
| Brain | rno-let-7b-5p | MIMAT0000775 | 18258830 |
| Brain | rno-let-7c-5p | MIMAT0000776 | 18258830 |
| Brain | rno-let-7d-3p | MIMAT0000563 | 18258830 |
| Brain | rno-let-7d-5p | MIMAT0000562 | 19703567 |
| Brain | rno-let-7d-5p | MIMAT0000562 | 18258830 |
| Brain | rno-let-7d-5p | MIMAT0000562 | 20557304 |
| Brain | rno-let-7e-3p | MIMAT0004706 | 18258830 |
| Brain | rno-let-7e-5p | MIMAT0000777 | 18258830 |
| Brain | rno-let-7f-5p | MIMAT0000778 | 18258830 |
| Brain | rno-let-7i-3p | MIMAT0004707 | 18258830 |
| Brain | rno-let-7i-5p | MIMAT0000779 | 18258830 |
| Brain | rno-miR-100-5p | MIMAT0000822 | 18258830 |
| Brain | rno-miR-101a-3p | MIMAT0000823 | 18258830 |
| Brain | rno-miR-101b-3p | MIMAT0000615 | 18258830 |
| Brain | rno-miR-124-3p | MIMAT0000828 | 19703567 |
| Brain | rno-miR-124-3p | MIMAT0000828 | 19131573 |
| Brain | rno-miR-124-3p | MIMAT0000828 | 18258830 |
| Brain | rno-miR-125a-5p | MIMAT0000829 | 18258830 |
| Brain | rno-miR-129-5p | MIMAT0000600 | 18258830 |
| Brain | rno-miR-130a-3p | MIMAT0000836 | 18258830 |
| Brain | rno-miR-132-3p | MIMAT0000838 | 18258830 |
| Brain | rno-miR-134-5p | MIMAT0000840 | 16421561 |
| Brain | rno-miR-134-5p | MIMAT0000840 | 18258830 |
| Brain | rno-miR-135b-5p | MIMAT0000611 | 18667155 |
| Brain | rno-miR-138-5p | MIMAT0000844 | 18258830 |
| Brain | rno-miR-139-5p | MIMAT0000845 | 18258830 |
| Brain | rno-miR-145-5p | MIMAT0000851 | 19142192 |
| Brain | rno-miR-150-5p | MIMAT0000853 | 18258830 |
| Brain | rno-miR-181a-5p | MIMAT0000858 | 22144581 |
| Brain | rno-miR-181b-5p | MIMAT0000859 | 18258830 |
| Brain | rno-miR-183-5p | MIMAT0000860 | 19711202 |
| Brain | rno-miR-185-5p | MIMAT0000862 | 18258830 |
| Brain | rno-miR-18a-5p | MIMAT0000787 | 19131573 |
| Brain | rno-miR-204-5p | MIMAT0000877 | 18258830 |
| Brain | rno-miR-21-5p | MIMAT0000790 | 21154462 |
| Brain | rno-miR-21-5p | MIMAT0000790 | 18258830 |
| Brain | rno-miR-214-3p | MIMAT0000885 | 18258830 |
| Brain | rno-miR-215 | MIMAT0003118 | 18258830 |
| Brain | rno-miR-218a-5p | MIMAT0000888 | 18258830 |
| Brain | rno-miR-223-3p | MIMAT0000892 | 18258830 |
| Brain | rno-miR-23a-3p | MIMAT0000792 | 18258830 |
| Brain | rno-miR-23b-3p | MIMAT0000793 | 18258830 |
| Brain | rno-miR-24-3p | MIMAT0000794 | 18258830 |
| Brain | rno-miR-25-3p | MIMAT0000795 | 18258830 |
| Brain | rno-miR-26b-5p | MIMAT0000797 | 18258830 |
| Brain | rno-miR-27a-3p | MIMAT0000799 | 18258830 |
| Brain | rno-miR-290 | MIMAT0000893 | 18258830 |
| Brain | rno-miR-292-5p | MIMAT0000896 | 18258830 |
| Brain | rno-miR-293-5p | MIMAT0012847 | 18258830 |
| Brain | rno-miR-298-5p | MIMAT0000900 | 18258830 |
| Brain | rno-miR-29a-3p | MIMAT0000802 | 18258830 |
| Brain | rno-miR-29b-3p | MIMAT0000801 | 18258830 |
| Brain | rno-miR-29c-3p | MIMAT0000803 | 18258830 |
| Brain | rno-miR-301a-3p | MIMAT0000552 | 18258830 |
| Brain | rno-miR-30a-3p | MIMAT0000809 | 18258830 |
| Brain | rno-miR-30a-5p | MIMAT0000808 | 18258830 |
| Brain | rno-miR-30b-5p | MIMAT0000806 | 18258830 |
| Brain | rno-miR-30c-5p | MIMAT0000804 | 18258830 |
| Brain | rno-miR-30e-5p | MIMAT0000805 | 18258830 |
| Brain | rno-miR-320-3p | MIMAT0000903 | 18258830 |
| Brain | rno-miR-322-5p | MIMAT0001619 | 18258830 |
| Brain | rno-miR-324-5p | MIMAT0000553 | 18258830 |
| Brain | rno-miR-327 | MIMAT0000561 | 18258830 |
| Brain | rno-miR-328a-3p | MIMAT0000564 | 18258830 |
| Brain | rno-miR-331-3p | MIMAT0000570 | 18258830 |
| Brain | rno-miR-338-3p | MIMAT0000581 | 18258830 |
| Brain | rno-miR-342-3p | MIMAT0000589 | 18258830 |
| Brain | rno-miR-342-5p | MIMAT0004652 | 18258830 |
| Brain | rno-miR-34a-5p | MIMAT0000815 | 18258830 |
| Brain | rno-miR-374-5p | MIMAT0003208 | 18258830 |
| Brain | rno-miR-382-5p | MIMAT0003201 | 18258830 |
| Brain | rno-miR-383-5p | MIMAT0003114 | 18258830 |
| Brain | rno-miR-409a-3p | MIMAT0003205 | 18258830 |
| Brain | rno-miR-485-5p | MIMAT0003203 | 18258830 |
| Brain | rno-miR-494-3p | MIMAT0003193 | 18258830 |
| Brain | rno-miR-497-5p | MIMAT0003383 | 18258830 |
| Brain | rno-miR-543-5p | MIMAT0004787 | 18258830 |
| Brain | rno-miR-664-3p | MIMAT0003382 | 18258830 |
| Brain | rno-miR-7a-5p | MIMAT0000606 | 18258830 |
| Brain | rno-miR-92a-3p | MIMAT0000816 | 18258830 |
| Brain | rno-miR-98-5p | MIMAT0000819 | 18258830 |
| Brain | rno-miR-99a-5p | MIMAT0000820 | 18258830 |
| Brain | rno-miR-99b-5p | MIMAT0000821 | 18258830 |
| Brain | rno-miR-9a-5p | MIMAT0000781 | 18667155 |
| Brain | rno-miR-9a-5p | MIMAT0000781 | 18258830 |
| Breast | bta-miR-335 | MIMAT0009291 | 18185580 |
| Breast | cfa-miR-27a | MIMAT0006641 | 18006846 |
| Breast | cfa-miR-335 | MIMAT0006624 | 18185580 |
| Breast | dre-miR-27a | MIMAT0001796 | 18006846 |
| Breast | hsa-let-7a-5p | MIMAT0000062 | 18083101 |
| Breast | hsa-let-7a-5p | MIMAT0000062 | 18758960 |
| Breast | hsa-let-7a-5p | MIMAT0000062 | 19878981 |
| Breast | hsa-let-7f-5p | MIMAT0000067 | 22407818 |
| Breast | hsa-miR-103a-3p | MIMAT0000101 | 20603000 |
| Breast | hsa-miR-106a-5p | MIMAT0000103 | 16461460 |
| Breast | hsa-miR-107 | MIMAT0000104 | 20603000 |
| Breast | hsa-miR-10b-5p | MIMAT0000254 | 17898713 |
| Breast | hsa-miR-10b-5p | MIMAT0000254 | 22573479 |
| Breast | hsa-miR-1226-3p | MIMAT0005577 | 20514397 |
| Breast | hsa-miR-1258 | MIMAT0005909 | 21266359 |
| Breast | hsa-miR-125a-5p | MIMAT0000443 | 19875930 |
| Breast | hsa-miR-125a-5p | MIMAT0000443 | 21220473 |
| Breast | hsa-miR-125b-5p | MIMAT0000423 | 19875930 |
| Breast | hsa-miR-125b-5p | MIMAT0000423 | 19570947 |
| Breast | hsa-miR-125b-5p | MIMAT0000423 | 20729973 |
| Breast | hsa-miR-125b-5p | MIMAT0000423 | 19738052 |
| Breast | hsa-miR-128-3p | MIMAT0000424 | 20054641 |
| Breast | hsa-miR-141-3p | MIMAT0000432 | 18483486 |
| Breast | hsa-miR-145-5p | MIMAT0000437 | 21092188 |
| Breast | hsa-miR-145-5p | MIMAT0000437 | 19360360 |
| Breast | hsa-miR-145-5p | MIMAT0000437 | 20668064 |
| Breast | hsa-miR-145-5p | MIMAT0000437 | 19996288 |
| Breast | hsa-miR-146a-5p | MIMAT0000449 | 18660546 |
| Breast | hsa-miR-146a-5p | MIMAT0000449 | 18504431 |
| Breast | hsa-miR-146b-5p | MIMAT0002809 | 18504431 |
| Breast | hsa-miR-150-5p | MIMAT0000451 | 18667440 |
| Breast | hsa-miR-153-3p | MIMAT0000439 | 20668064 |
| Breast | hsa-miR-154-5p | MIMAT0000452 | 20603000 |
| Breast | hsa-miR-155-5p | MIMAT0000646 | 20354188 |
| Breast | hsa-miR-155-5p | MIMAT0000646 | 20371610 |
| Breast | hsa-miR-155-5p | MIMAT0000646 | 18794355 |
| Breast | hsa-miR-155-5p | MIMAT0000646 | 20427544 |
| Breast | hsa-miR-16-5p | MIMAT0000069 | 20668064 |
| Breast | hsa-miR-17-5p | MIMAT0000070 | 18695042 |
| Breast | hsa-miR-17-5p | MIMAT0000070 | 16940181 |
| Breast | hsa-miR-17-5p | MIMAT0000070 | 20406904 |
| Breast | hsa-miR-181b-5p | MIMAT0000257 | 22009755 |
| Breast | hsa-miR-182-5p | MIMAT0000259 | 19574223 |
| Breast | hsa-miR-182-5p | MIMAT0000259 | 22322863 |
| Breast | hsa-miR-182-5p | MIMAT0000259 | 23430586 |
| Breast | hsa-miR-185-5p | MIMAT0000455 | 20603620 |
| Breast | hsa-miR-188-5p | MIMAT0000457 | 19223510 |
| Breast | hsa-miR-18a-5p | MIMAT0000072 | 19706389 |
| Breast | hsa-miR-193b-3p | MIMAT0002819 | 19701247 |
| Breast | hsa-miR-196a-5p | MIMAT0000226 | 20736365 |
| Breast | hsa-miR-196a-5p | MIMAT0000226 | 18663355 |
| Breast | hsa-miR-196b-5p | MIMAT0001080 | 20736365 |
| Breast | hsa-miR-198 | MIMAT0000228 | 18667440 |
| Breast | hsa-miR-199a-3p | MIMAT0000232 | 21807947 |
| Breast | hsa-miR-199b-5p | MIMAT0000263 | 23354452 |
| Breast | hsa-miR-19b-3p | MIMAT0000074 | 19706389 |
| Breast | hsa-miR-200a-3p | MIMAT0000682 | 18376396 |
| Breast | hsa-miR-200b-3p | MIMAT0000318 | 18376396 |
| Breast | hsa-miR-200b-3p | MIMAT0000318 | 19839049 |
| Breast | hsa-miR-200b-3p | MIMAT0000318 | 22144583 |
| Breast | hsa-miR-200c-3p | MIMAT0000617 | 19665978 |
| Breast | hsa-miR-200c-3p | MIMAT0000617 | 19223510 |
| Breast | hsa-miR-200c-3p | MIMAT0000617 | 19839049 |
| Breast | hsa-miR-200c-3p | MIMAT0000617 | 19435871 |
| Breast | hsa-miR-200c-3p | MIMAT0000617 | 18483486 |
| Breast | hsa-miR-203a | MIMAT0000264 | 20668064 |
| Breast | hsa-miR-204-5p | MIMAT0000265 | 18922924 |
| Breast | hsa-miR-204-5p | MIMAT0000265 | 19074899 |
| Breast | hsa-miR-205-5p | MIMAT0000266 | 18376396 |
| Breast | hsa-miR-205-5p | MIMAT0000266 | 19276373 |
| Breast | hsa-miR-205-5p | MIMAT0000266 | 22578566 |
| Breast | hsa-miR-206 | MIMAT0000462 | 17312270 |
| Breast | hsa-miR-206 | MIMAT0000462 | 18593897 |
| Breast | hsa-miR-206 | MIMAT0000462 | 20388878 |
| Breast | hsa-miR-206 | MIMAT0000462 | 19414598 |
| Breast | hsa-miR-20a-5p | MIMAT0000075 | 16461460 |
| Breast | hsa-miR-20a-5p | MIMAT0000075 | 18695042 |
| Breast | hsa-miR-20a-5p | MIMAT0000075 | 20406904 |
| Breast | hsa-miR-20b-5p | MIMAT0001413 | 20232316 |
| Breast | hsa-miR-20b-5p | MIMAT0001413 | 19706389 |
| Breast | hsa-miR-21-5p | MIMAT0000076 | 19264808 |
| Breast | hsa-miR-21-5p | MIMAT0000076 | 19473551 |
| Breast | hsa-miR-21-5p | MIMAT0000076 | 20346171 |
| Breast | hsa-miR-21-5p | MIMAT0000076 | 19302977 |
| Breast | hsa-miR-21-5p | MIMAT0000076 | 21047769 |
| Breast | hsa-miR-21-5p | MIMAT0000076 | 17072344 |
| Breast | hsa-miR-21-5p | MIMAT0000076 | 17363372 |
| Breast | hsa-miR-21-5p | MIMAT0000076 | 20460403 |
| Breast | hsa-miR-21-5p | MIMAT0000076 | 19419954 |
| Breast | hsa-miR-21-5p | MIMAT0000076 | 19528081 |
| Breast | hsa-miR-21-5p | MIMAT0000076 | 18270520 |
| Breast | hsa-miR-21-5p | MIMAT0000076 | 18372920 |
| Breast | hsa-miR-21-5p | MIMAT0000076 | 17991735 |
| Breast | hsa-miR-21-5p | MIMAT0000076 | 19253296 |
| Breast | hsa-miR-210-3p | MIMAT0000267 | 19652553 |
| Breast | hsa-miR-216a-5p | MIMAT0000273 | 21149267 |
| Breast | hsa-miR-217 | MIMAT0000274 | 20668064 |
| Breast | hsa-miR-22-3p | MIMAT0000077 | 19414598 |
| Breast | hsa-miR-22-3p | MIMAT0000077 | 20180843 |
| Breast | hsa-miR-221-3p | MIMAT0000278 | 18708351 |
| Breast | hsa-miR-221-3p | MIMAT0000278 | 22009755 |
| Breast | hsa-miR-221-3p | MIMAT0000278 | 18790736 |
| Breast | hsa-miR-221-3p | MIMAT0000278 | 20388878 |
| Breast | hsa-miR-222-3p | MIMAT0000279 | 18708351 |
| Breast | hsa-miR-222-3p | MIMAT0000279 | 18790736 |
| Breast | hsa-miR-222-3p | MIMAT0000279 | 20388878 |
| Breast | hsa-miR-222-3p | MIMAT0000279 | 22009755 |
| Breast | hsa-miR-224-5p | MIMAT0000281 | 20180642 |
| Breast | hsa-miR-26a-5p | MIMAT0000082 | 16461460 |
| Breast | hsa-miR-27a-3p | MIMAT0000084 | 20382698 |
| Breast | hsa-miR-27a-3p | MIMAT0000084 | 18006846 |
| Breast | hsa-miR-27a-3p | MIMAT0000084 | 19574223 |
| Breast | hsa-miR-27b-3p | MIMAT0000419 | 16982751 |
| Breast | hsa-miR-299-5p | MIMAT0002890 | 19538464 |
| Breast | hsa-miR-29a-3p | MIMAT0000086 | 22864815 |
| Breast | hsa-miR-29b-3p | MIMAT0000100 | 21359530 |
| Breast | hsa-miR-29b-3p | MIMAT0000100 | 19706389 |
| Breast | hsa-miR-30c-5p | MIMAT0000244 | 19223510 |
| Breast | hsa-miR-30e-5p | MIMAT0000692 | 19223510 |
| Breast | hsa-miR-31-3p | MIMAT0004504 | 19524507 |
| Breast | hsa-miR-31-5p | MIMAT0000089 | 19524507 |
| Breast | hsa-miR-326 | MIMAT0000756 | 19883630 |
| Breast | hsa-miR-328-3p | MIMAT0000752 | 19270061 |
| Breast | hsa-miR-328-5p | MIMAT0026486 | 19270061 |
| Breast | hsa-miR-330-3p | MIMAT0000751 | 21149267 |
| Breast | hsa-miR-335-5p | MIMAT0000765 | 18185580 |
| Breast | hsa-miR-335-5p | MIMAT0000765 | 21618216 |
| Breast | hsa-miR-339-5p | MIMAT0000764 | 20932331 |
| Breast | hsa-miR-342-3p | MIMAT0000753 | 21172025 |
| Breast | hsa-miR-345-5p | MIMAT0000772 | 20099276 |
| Breast | hsa-miR-34a-5p | MIMAT0000255 | 23292869 |
| Breast | hsa-miR-34a-5p | MIMAT0000255 | 21240262 |
| Breast | hsa-miR-34a-5p | MIMAT0000255 | 21814748 |
| Breast | hsa-miR-373-3p | MIMAT0000726 | 18193036 |
| Breast | hsa-miR-373-3p | MIMAT0000726 | 21271679 |
| Breast | hsa-miR-375 | MIMAT0000728 | 20978187 |
| Breast | hsa-miR-448 | MIMAT0001532 | 20798686 |
| Breast | hsa-miR-451a | MIMAT0001631 | 18645025 |
| Breast | hsa-miR-483-3p | MIMAT0002173 | 20388800 |
| Breast | hsa-miR-494-3p | MIMAT0002816 | 22544933 |
| Breast | hsa-miR-510-5p | MIMAT0002882 | 18922924 |
| Breast | hsa-miR-520b | MIMAT0002843 | 20574151 |
| Breast | hsa-miR-520c-3p | MIMAT0002846 | 18193036 |
| Breast | hsa-miR-520e | MIMAT0002825 | 20574151 |
| Breast | hsa-miR-559 | MIMAT0003223 | 19584269 |
| Breast | hsa-miR-608 | MIMAT0003276 | 21149267 |
| Breast | hsa-miR-632 | MIMAT0003302 | 17980029 |
| Breast | hsa-miR-661 | MIMAT0003324 | 19584269 |
| Breast | hsa-miR-661 | MIMAT0003324 | 20543867 |
| Breast | hsa-miR-7-5p | MIMAT0000252 | 19073608 |
| Breast | hsa-miR-7-5p | MIMAT0000252 | 20099276 |
| Breast | hsa-miR-7-5p | MIMAT0000252 | 18922890 |
| Breast | hsa-miR-9-3p | MIMAT0000442 | 23530058 |
| Breast | hsa-miR-9-5p | MIMAT0000441 | 20173740 |
| Breast | hsa-miR-92a-3p | MIMAT0000092 | 20484043 |
| Breast | hsa-miR-96-5p | MIMAT0000095 | 21203424 |
| Breast | hsa-miR-96-5p | MIMAT0000095 | 19574223 |
| Breast | hsa-miR-98-5p | MIMAT0000096 | 19528081 |
| Breast | mmu-miR-10b-5p | MIMAT0000208 | 20351690 |
| Breast | mmu-miR-155-5p | MIMAT0000165 | 18794355 |
| Breast | mmu-miR-200b-3p | MIMAT0000233 | 19787069 |
| Breast | mmu-miR-200c-3p | MIMAT0000657 | 19787069 |
| Breast | mmu-miR-21a-5p | MIMAT0000530 | 18372920 |
| Breast | mmu-miR-24-2-5p | MIMAT0005440 | 22911661 |
| Breast | mmu-miR-465a-5p | MIMAT0002106 | 18922890 |
| Breast | ppy-miR-27a | MIMAT0002748 | 18006846 |
| Breast | ppy-miR-335 | MIMAT0015837 | 18185580 |
| Breast | ptr-miR-27a | MIMAT0002750 | 18006846 |
| Breast | ptr-miR-335 | MIMAT0008104 | 18185580 |
| Breast | ptr-miR-520c | MIMAT0008199 | 18193036 |
| Breast | rno-miR-200c-3p | MIMAT0000873 | 22144583 |
| Breast | rno-miR-335 | MIMAT0000575 | 18185580 |
| Breast | ssc-miR-27a | MIMAT0002148 | 18006846 |
| Breast | ssc-miR-335 | MIMAT0013955 | 18185580 |
| Breast | xtr-miR-27a | MIMAT0003570 | 18006846 |
| CA1 Region Hippocampal | hsa-miR-183-5p | MIMAT0000261 | 19711202 |
| CA1 Region Hippocampal | rno-miR-183-5p | MIMAT0000860 | 19711202 |
| Capillaries | hsa-miR-29b-3p | MIMAT0000100 | 21793034 |
| Cardiovascular System | hsa-miR-195-5p | MIMAT0000461 | 22802111 |
| Cardiovascular System | mmu-miR-27b-3p | MIMAT0000126 | 20736237 |
| Carotid Arteries | hsa-miR-1 | MIMAT0000416 | 21051663 |
| Carotid Arteries | hsa-miR-195-5p | MIMAT0000461 | 22802111 |
| Carotid Arteries | hsa-miR-21-5p | MIMAT0000076 | 22034194 |
| Carotid Arteries | hsa-miR-21-5p | MIMAT0000076 | 17478730 |
| Carotid Arteries | hsa-miR-221-3p | MIMAT0000278 | 19150885 |
| Carotid Arteries | hsa-miR-222-3p | MIMAT0000279 | 19150885 |
| Carotid Arteries | hsa-miR-616-3p | MIMAT0004805 | 23497787 |
| Carotid Arteries | rno-miR-145-5p | MIMAT0000851 | 19542014 |
| Carotid Arteries | rno-miR-21-5p | MIMAT0000790 | 17478730 |
| Carotid Arteries | rno-miR-221-3p | MIMAT0000890 | 19150885 |
| Carotid Arteries | rno-miR-222-3p | MIMAT0000891 | 19150885 |
| Carotid Arteries | rno-miR-31a-5p | MIMAT0000810 | 19542014 |
| Carotid Arteries | rno-miR-31a-5p | MIMAT0000810 | 22020941 |
| Carotid Artery Internal | hsa-miR-616-3p | MIMAT0004805 | 23497787 |
| Cartilage | hsa-miR-22-3p | MIMAT0000077 | 19011694 |
| Cartilage | hsa-miR-9-5p | MIMAT0000441 | 19008124 |
| Cartilage | mmu-miR-125b-5p | MIMAT0000136 | 21945074 |
| Cartilage | mmu-miR-140-5p | MIMAT0000151 | 16828749 |
| Cartilage | mmu-miR-140-5p | MIMAT0000151 | 21872590 |
| Cartilage | mmu-miR-199a-3p | MIMAT0000230 | 19251704 |
| Cartilage | mmu-miR-30a-5p | MIMAT0000128 | 21945074 |
| Cartilage | mmu-miR-30c-5p | MIMAT0000514 | 21945074 |
| Cell Membrane | hsa-miR-1 | MIMAT0000416 | 17401374 |
| Cell Membrane | hsa-miR-204-5p | MIMAT0000265 | 20056717 |
| Cell Membrane | hsa-miR-21-5p | MIMAT0000076 | 20056717 |
| Cell Membrane | rno-miR-1-3p | MIMAT0003125 | 17401374 |
| Central Nervous System | hsa-miR-1 | MIMAT0000416 | 21169019 |
| Central Nervous System | hsa-miR-124-3p | MIMAT0000422 | 18577219 |
| Central Nervous System | hsa-miR-124-3p | MIMAT0000422 | 23318130 |
| Central Nervous System | hsa-miR-125a-5p | MIMAT0000443 | 21220473 |
| Central Nervous System | hsa-miR-133b | MIMAT0000770 | 17761882 |
| Central Nervous System | hsa-miR-137 | MIMAT0000429 | 18577219 |
| Central Nervous System | hsa-miR-19b-3p | MIMAT0000074 | 20435064 |
| Central Nervous System | hsa-miR-29a-3p | MIMAT0000086 | 19584290 |
| Central Nervous System | hsa-miR-29a-3p | MIMAT0000086 | 21169019 |
| Central Nervous System | hsa-miR-302b-5p | MIMAT0000714 | 20435064 |
| Central Nervous System | hsa-miR-323a-3p | MIMAT0000755 | 20435064 |
| Central Nervous System | mmu-miR-124-3p | MIMAT0000134 | 18577219 |
| Central Nervous System | mmu-miR-124-3p | MIMAT0000134 | 21131957 |
| Central Nervous System | mmu-miR-134-5p | MIMAT0000146 | 20622856 |
| Central Nervous System | mmu-miR-137-3p | MIMAT0000149 | 18577219 |
| Central Nervous System | mmu-miR-137-5p | MIMAT0016986 | 18577219 |
| Central Nervous System | mmu-miR-155-5p | MIMAT0000165 | 21247879 |
| Central Nervous System | mmu-miR-23a-3p | MIMAT0000532 | 19259393 |
| Central Nervous System | mmu-miR-23b-3p | MIMAT0000125 | 19259393 |
| Central Nervous System | mmu-miR-298-5p | MIMAT0000376 | 21247879 |
| Central Nervous System | mmu-miR-351-5p | MIMAT0000609 | 21247879 |
| Central Nervous System | mmu-miR-9-5p | MIMAT0000142 | 21238922 |
| Central Nervous System | rno-miR-140-3p | MIMAT0000574 | 18845019 |
| Central Nervous System | rno-miR-34a-5p | MIMAT0000815 | 18704095 |
| Cerebellum | hsa-miR-124-3p | MIMAT0000422 | 18607543 |
| Cerebellum | hsa-miR-199b-5p | MIMAT0000263 | 19308264 |
| Cerebral Cortex | hsa-miR-29a-3p | MIMAT0000086 | 20202123 |
| Cerebral Cortex | hsa-miR-34a-5p | MIMAT0000255 | 19683563 |
| Cerebral Cortex | mmu-miR-34a-5p | MIMAT0000542 | 19683563 |
| Cervix Uteri | hsa-miR-218-5p | MIMAT0000275 | 17998940 |
| CHO Cells | mmu-miR-466h-5p | MIMAT0004884 | 21337326 |
| Chondrocytes | hsa-miR-140-5p | MIMAT0000431 | 21576357 |
| Chondrocytes | hsa-miR-140-5p | MIMAT0000431 | 19948051 |
| Chondrocytes | hsa-miR-199a-5p | MIMAT0000231 | 22441842 |
| Chondrocytes | hsa-miR-22-3p | MIMAT0000077 | 19011694 |
| Chondrocytes | hsa-miR-27a-3p | MIMAT0000084 | 19948051 |
| Chondrocytes | hsa-miR-27b-3p | MIMAT0000419 | 20131257 |
| Chondrocytes | hsa-miR-29a-3p | MIMAT0000086 | 21665270 |
| Chondrocytes | hsa-miR-29b-3p | MIMAT0000100 | 21665270 |
| Chondrocytes | hsa-miR-9-5p | MIMAT0000441 | 19008124 |
| Chondrocytes | mmu-miR-125b-5p | MIMAT0000136 | 21945074 |
| Chondrocytes | mmu-miR-140-5p | MIMAT0000151 | 21872590 |
| Chondrocytes | mmu-miR-140-5p | MIMAT0000151 | 21576357 |
| Chondrocytes | mmu-miR-29a-3p | MIMAT0000535 | 21665270 |
| Chondrocytes | mmu-miR-29b-3p | MIMAT0000127 | 21665270 |
| Chondrocytes | mmu-miR-30a-5p | MIMAT0000128 | 21945074 |
| Chondrocytes | mmu-miR-30c-5p | MIMAT0000514 | 21945074 |
| Choroid | hsa-miR-204-5p | MIMAT0000265 | 20056717 |
| Choroid | hsa-miR-21-5p | MIMAT0000076 | 20056717 |
| Choroid | mmu-miR-449a-5p | MIMAT0001542 | 19056356 |
| Choroid Plexus | mmu-miR-449a-5p | MIMAT0001542 | 19056356 |
| Cilia | rno-miR-126a-5p | MIMAT0000831 | 19102782 |
| Cilia | rno-miR-128-1-5p | MIMAT0017118 | 19102782 |
| Cilia | rno-miR-136-5p | MIMAT0000842 | 19102782 |
| Cilia | rno-miR-147 | MIMAT0005297 | 19102782 |
| Cilia | rno-miR-181b-5p | MIMAT0000859 | 19102782 |
| Cilia | rno-miR-196a-5p | MIMAT0000871 | 19102782 |
| Cilia | rno-miR-203a-5p | MIMAT0017153 | 19102782 |
| Cilia | rno-miR-20a-5p | MIMAT0000602 | 19102782 |
| Cilia | rno-miR-21-5p | MIMAT0000790 | 19102782 |
| Cilia | rno-miR-216a-5p | MIMAT0000886 | 19102782 |
| Cilia | rno-miR-217-5p | MIMAT0000887 | 19102782 |
| Cilia | rno-miR-30a-5p | MIMAT0000808 | 19102782 |
| Cilia | rno-miR-31a-5p | MIMAT0000810 | 19102782 |
| Cilia | rno-miR-335 | MIMAT0000575 | 19102782 |
| Cilia | rno-miR-346 | MIMAT0000596 | 19102782 |
| Cilia | rno-miR-34b-5p | MIMAT0000813 | 19102782 |
| Cilia | rno-miR-377-5p | MIMAT0017203 | 19102782 |
| Cilia | rno-miR-379-5p | MIMAT0003192 | 19102782 |
| Cilia | rno-miR-380-5p | MIMAT0005308 | 19102782 |
| Cilia | rno-miR-448-5p | MIMAT0017177 | 19102782 |
| Cilia | rno-miR-7a-5p | MIMAT0000606 | 19102782 |
| Cilia | rno-miR-7b | MIMAT0000780 | 19102782 |
| Cilia | rno-miR-96-5p | MIMAT0000818 | 19102782 |
| Cilia | rno-miR-99a-5p | MIMAT0000820 | 19102782 |
| Clone Cells | hsa-miR-196a-5p | MIMAT0000226 | 21077158 |
| Clone Cells | hsa-miR-199b-5p | MIMAT0000263 | 21557766 |
| Clone Cells | mmu-miR-106b-5p | MIMAT0000386 | 21285944 |
| Clone Cells | mmu-miR-216a-5p | MIMAT0000662 | 20713358 |
| Clone Cells | mmu-miR-93-5p | MIMAT0000540 | 21285944 |
| Cochlea | mmu-miR-15a-5p | MIMAT0000526 | 19416898 |
| Colon | bta-miR-143 | MIMAT0009233 | 17504027 |
| Colon | cfa-miR-143 | MIMAT0006682 | 17504027 |
| Colon | dre-miR-143 | MIMAT0001840 | 17504027 |
| Colon | hsa-let-7a-5p | MIMAT0000062 | 16651716 |
| Colon | hsa-let-7a-5p | MIMAT0000062 | 17942906 |
| Colon | hsa-let-7b-3p | MIMAT0004482 | 18379589 |
| Colon | hsa-let-7b-5p | MIMAT0000063 | 18379589 |
| Colon | hsa-let-7b-5p | MIMAT0000063 | 17942906 |
| Colon | hsa-miR-106a-5p | MIMAT0000103 | 16461460 |
| Colon | hsa-miR-106b-5p | MIMAT0000680 | 21283757 |
| Colon | hsa-miR-107 | MIMAT0000104 | 20308559 |
| Colon | hsa-miR-126-3p | MIMAT0000445 | 18663744 |
| Colon | hsa-miR-128-3p | MIMAT0000424 | 20388705 |
| Colon | hsa-miR-137 | MIMAT0000429 | 20682795 |
| Colon | hsa-miR-137 | MIMAT0000429 | 23400681 |
| Colon | hsa-miR-140-5p | MIMAT0000431 | 19734943 |
| Colon | hsa-miR-141-3p | MIMAT0000432 | 18835392 |
| Colon | hsa-miR-143-3p | MIMAT0000435 | 17892514 |
| Colon | hsa-miR-143-3p | MIMAT0000435 | 19638978 |
| Colon | hsa-miR-143-3p | MIMAT0000435 | 19843160 |
| Colon | hsa-miR-143-3p | MIMAT0000435 | 19439999 |
| Colon | hsa-miR-143-3p | MIMAT0000435 | 19464056 |
| Colon | hsa-miR-143-3p | MIMAT0000435 | 16969504 |
| Colon | hsa-miR-143-3p | MIMAT0000435 | 17504027 |
| Colon | hsa-miR-143-3p | MIMAT0000435 | 21276449 |
| Colon | hsa-miR-145-5p | MIMAT0000437 | 21092188 |
| Colon | hsa-miR-145-5p | MIMAT0000437 | 20098684 |
| Colon | hsa-miR-145-5p | MIMAT0000437 | 20687965 |
| Colon | hsa-miR-145-5p | MIMAT0000437 | 20737575 |
| Colon | hsa-miR-145-5p | MIMAT0000437 | 19391107 |
| Colon | hsa-miR-155-5p | MIMAT0000646 | 20427544 |
| Colon | hsa-miR-15b-5p | MIMAT0000417 | 16609010 |
| Colon | hsa-miR-181b-5p | MIMAT0000257 | 20797623 |
| Colon | hsa-miR-183-5p | MIMAT0000261 | 21118966 |
| Colon | hsa-miR-186-5p | MIMAT0000456 | 23137536 |
| Colon | hsa-miR-18a-3p | MIMAT0002891 | 19372139 |
| Colon | hsa-miR-192-5p | MIMAT0000222 | 18835392 |
| Colon | hsa-miR-192-5p | MIMAT0000222 | 19088023 |
| Colon | hsa-miR-195-5p | MIMAT0000461 | 23526568 |
| Colon | hsa-miR-199b-5p | MIMAT0000263 | 23354452 |
| Colon | hsa-miR-200a-3p | MIMAT0000682 | 18835392 |
| Colon | hsa-miR-20a-5p | MIMAT0000075 | 16461460 |
| Colon | hsa-miR-21-5p | MIMAT0000076 | 19826040 |
| Colon | hsa-miR-21-5p | MIMAT0000076 | 19836969 |
| Colon | hsa-miR-21-5p | MIMAT0000076 | 19302977 |
| Colon | hsa-miR-21-5p | MIMAT0000076 | 21069438 |
| Colon | hsa-miR-21-5p | MIMAT0000076 | 22072622 |
| Colon | hsa-miR-21-5p | MIMAT0000076 | 18372920 |
| Colon | hsa-miR-21-5p | MIMAT0000076 | 20797623 |
| Colon | hsa-miR-21-5p | MIMAT0000076 | 18508928 |
| Colon | hsa-miR-212-3p | MIMAT0000269 | 18162065 |
| Colon | hsa-miR-216b-5p | MIMAT0004959 | 23137536 |
| Colon | hsa-miR-217 | MIMAT0000274 | 18835392 |
| Colon | hsa-miR-221-3p | MIMAT0000278 | 21278784 |
| Colon | hsa-miR-25-3p | MIMAT0000081 | 23435373 |
| Colon | hsa-miR-26a-5p | MIMAT0000082 | 16461460 |
| Colon | hsa-miR-27b-3p | MIMAT0000419 | 20388705 |
| Colon | hsa-miR-27b-3p | MIMAT0000419 | 18835392 |
| Colon | hsa-miR-29a-3p | MIMAT0000086 | 19951903 |
| Colon | hsa-miR-320a | MIMAT0000510 | 22134529 |
| Colon | hsa-miR-337-3p | MIMAT0000754 | 23137536 |
| Colon | hsa-miR-34a-5p | MIMAT0000255 | 17875987 |
| Colon | hsa-miR-34a-5p | MIMAT0000255 | 18755897 |
| Colon | hsa-miR-34a-5p | MIMAT0000255 | 21240262 |
| Colon | hsa-miR-34a-5p | MIMAT0000255 | 20433755 |
| Colon | hsa-miR-362-3p | MIMAT0004683 | 23280316 |
| Colon | hsa-miR-365a-3p | MIMAT0000710 | 22072615 |
| Colon | hsa-miR-483-3p | MIMAT0002173 | 20388800 |
| Colon | hsa-miR-499a-5p | MIMAT0002870 | 21934092 |
| Colon | hsa-miR-499b-5p | MIMAT0019897 | 21934092 |
| Colon | hsa-miR-519a-3p | MIMAT0002869 | 19088191 |
| Colon | hsa-miR-519b-3p | MIMAT0002837 | 19088191 |
| Colon | hsa-miR-519c-3p | MIMAT0002832 | 19088191 |
| Colon | hsa-miR-519c-3p | MIMAT0002832 | 18573883 |
| Colon | hsa-miR-532-5p | MIMAT0002888 | 18835392 |
| Colon | hsa-miR-562 | MIMAT0003226 | 19789318 |
| Colon | hsa-miR-603 | MIMAT0003271 | 18835392 |
| Colon | hsa-miR-630 | MIMAT0003299 | 23435373 |
| Colon | hsa-miR-633 | MIMAT0003303 | 20637737 |
| Colon | hsa-miR-675-5p | MIMAT0004284 | 19926638 |
| Colon | hsa-miR-760 | MIMAT0004957 | 23137536 |
| Colon | hsa-miR-769-5p | MIMAT0003886 | 18835392 |
| Colon | hsa-miR-98-5p | MIMAT0000096 | 17942906 |
| Colon | mmu-miR-128-3p | MIMAT0000140 | 20388705 |
| Colon | mmu-miR-140-5p | MIMAT0000151 | 19734943 |
| Colon | mmu-miR-21a-5p | MIMAT0000530 | 18508928 |
| Colon | mmu-miR-21a-5p | MIMAT0000530 | 18372920 |
| Colon | mmu-miR-223-3p | MIMAT0000665 | 22043014 |
| Colon | mmu-miR-27b-3p | MIMAT0000126 | 20388705 |
| Colon | ppy-miR-143 | MIMAT0002259 | 17504027 |
| Colon | ptr-miR-143 | MIMAT0002257 | 17504027 |
| Colon | rno-let-7a-5p | MIMAT0000774 | 17942906 |
| Colon | xtr-miR-143 | MIMAT0003686 | 17504027 |
| Connective Tissue | hsa-miR-146a-5p | MIMAT0000449 | 20375304 |
| Connective Tissue | hsa-miR-18a-5p | MIMAT0000072 | 23249750 |
| Connective Tissue | rno-miR-192-5p | MIMAT0000867 | 20393144 |
| Connective Tissue | rno-miR-215 | MIMAT0003118 | 20393144 |
| Cornea | hsa-miR-126-3p | MIMAT0000445 | 18987025 |
| Cornea | mmu-miR-126a-3p | MIMAT0000138 | 18987025 |
| Cornea | mmu-miR-150-5p | MIMAT0000160 | 18500251 |
| Cornea | mmu-miR-184-3p | MIMAT0000213 | 18500251 |
| Cornea | mmu-miR-31-5p | MIMAT0000538 | 18500251 |
| Coronary Vessels | hsa-miR-1 | MIMAT0000416 | 17401374 |
| Coronary Vessels | hsa-miR-1 | MIMAT0000416 | 19775284 |
| Coronary Vessels | hsa-miR-216a-5p | MIMAT0000273 | 19786632 |
| Coronary Vessels | hsa-miR-217 | MIMAT0000274 | 19786632 |
| Coronary Vessels | hsa-miR-31-5p | MIMAT0000089 | 23518389 |
| Coronary Vessels | rno-miR-1-3p | MIMAT0003125 | 17401374 |
| Coronary Vessels | rno-miR-1-3p | MIMAT0003125 | 19775284 |
| Cytoplasm | hsa-let-7b-3p | MIMAT0004482 | 17437991 |
| Cytoplasm | hsa-let-7b-5p | MIMAT0000063 | 17437991 |
| Cytoplasm | hsa-let-7b-5p | MIMAT0000063 | 16971064 |
| Cytoplasm | hsa-let-7e-3p | MIMAT0004485 | 17437991 |
| Cytoplasm | hsa-let-7e-5p | MIMAT0000066 | 17437991 |
| Cytoplasm | hsa-miR-195-5p | MIMAT0000461 | 22217655 |
| Cytoplasm | hsa-miR-200c-3p | MIMAT0000617 | 22015043 |
| Cytoplasm | hsa-miR-29a-3p | MIMAT0000086 | 20041405 |
| Cytoplasm | hsa-miR-29b-3p | MIMAT0000100 | 20041405 |
| Cytoplasm | hsa-miR-29c-3p | MIMAT0000681 | 20041405 |
| Cytoplasm | hsa-miR-320a | MIMAT0000510 | 18852463 |
| Cytoplasm | hsa-miR-34a-5p | MIMAT0000255 | 20433755 |
| Cytoplasm | hsa-miR-9-3p | MIMAT0000442 | 19118166 |
| Cytoplasm | hsa-miR-9-5p | MIMAT0000441 | 19118166 |
| Cytoplasm | mmu-miR-124-3p | MIMAT0000134 | 19663910 |
| Cytoskeleton | hsa-miR-1 | MIMAT0000416 | 20571053 |
| Cytoskeleton | hsa-miR-126-3p | MIMAT0000445 | 20571053 |
| Cytoskeleton | hsa-miR-141-3p | MIMAT0000432 | 20584986 |
| Cytoskeleton | hsa-miR-196a-5p | MIMAT0000226 | 21077158 |
| Cytoskeleton | hsa-miR-200a-3p | MIMAT0000682 | 19801681 |
| Cytoskeleton | hsa-miR-200b-3p | MIMAT0000318 | 22144583 |
| Cytoskeleton | hsa-miR-200b-3p | MIMAT0000318 | 19801681 |
| Cytoskeleton | hsa-miR-21-5p | MIMAT0000076 | 20584986 |
| Cytoskeleton | hsa-miR-375 | MIMAT0000728 | 20584986 |
| Cytoskeleton | hsa-miR-429 | MIMAT0001536 | 19801681 |
| Cytoskeleton | hsa-miR-9-5p | MIMAT0000441 | 22131135 |
| Cytoskeleton | mmu-miR-124-3p | MIMAT0000134 | 18619591 |
| Cytoskeleton | mmu-miR-141-3p | MIMAT0000153 | 20584986 |
| Cytoskeleton | mmu-miR-375-3p | MIMAT0000739 | 20584986 |
| Cytoskeleton | rno-miR-141-3p | MIMAT0000846 | 20584986 |
| Cytoskeleton | rno-miR-200c-3p | MIMAT0000873 | 22144583 |
| Cytoskeleton | rno-miR-21-5p | MIMAT0000790 | 20584986 |
| Cytosol | hsa-miR-34a-5p | MIMAT0000255 | 20433755 |
| Dendrites | mmu-miR-134-5p | MIMAT0000146 | 20126250 |
| Dendrites | rno-miR-26a-5p | MIMAT0000796 | 17592044 |
| Dendritic Cells | hsa-let-7e-3p | MIMAT0004485 | 19398721 |
| Dendritic Cells | hsa-let-7e-5p | MIMAT0000066 | 19398721 |
| Dendritic Cells | hsa-let-7i-5p | MIMAT0000415 | 21742974 |
| Dendritic Cells | hsa-miR-146a-5p | MIMAT0000449 | 21236257 |
| Dendritic Cells | hsa-miR-146a-5p | MIMAT0000449 | 20375304 |
| Dendritic Cells | hsa-miR-155-3p | MIMAT0004658 | 20852130 |
| Dendritic Cells | hsa-miR-155-5p | MIMAT0000646 | 19386588 |
| Dendritic Cells | hsa-miR-155-5p | MIMAT0000646 | 20852130 |
| Dendritic Cells | hsa-miR-155-5p | MIMAT0000646 | 19193853 |
| Dendritic Cells | hsa-miR-181a-5p | MIMAT0000256 | 22956783 |
| Dendritic Cells | hsa-miR-181a-5p | MIMAT0000256 | 20841506 |
| Dendritic Cells | hsa-miR-21-5p | MIMAT0000076 | 19398721 |
| Dendritic Cells | hsa-miR-29a-3p | MIMAT0000086 | 21276447 |
| Dendritic Cells | hsa-miR-34a-5p | MIMAT0000255 | 19398721 |
| Dendritic Cells | mmu-miR-148a-3p | MIMAT0000516 | 21068402 |
| Dendritic Cells | mmu-miR-148b-3p | MIMAT0000580 | 21068402 |
| Dendritic Cells | mmu-miR-152-3p | MIMAT0000162 | 21068402 |
| Dendritic Cells | mmu-miR-155-5p | MIMAT0000165 | 17463290 |
| Dendritic Cells | mmu-miR-155-5p | MIMAT0000165 | 21385848 |
| Dendritic Cells | mmu-miR-155-5p | MIMAT0000165 | 19193853 |
| Dendritic Spines | hsa-miR-125b-5p | MIMAT0000423 | 20159450 |
| Dendritic Spines | hsa-miR-132-3p | MIMAT0000426 | 19850129 |
| Dendritic Spines | rno-miR-134-5p | MIMAT0000840 | 16421561 |
| Dentate Gyrus | rno-miR-21-5p | MIMAT0000790 | 21154462 |
| Dermis | hsa-miR-424-5p | MIMAT0001341 | 21179471 |
| Duodenum | mmu-miR-124-3p | MIMAT0000134 | 17462994 |
| Ear | hsa-miR-96-5p | MIMAT0000095 | 19363479 |
| Ear | mmu-miR-15a-5p | MIMAT0000526 | 19416898 |
| Ear Inner | hsa-miR-96-5p | MIMAT0000095 | 19363479 |
| Ear Inner | mmu-miR-15a-5p | MIMAT0000526 | 19416898 |
| Ectoderm | mmu-let-7a-5p | MIMAT0000521 | 19898466 |
| Ectoderm | mmu-let-7e-5p | MIMAT0000524 | 19898466 |
| Ectoderm | mmu-miR-125a-5p | MIMAT0000135 | 19898466 |
| Ectoderm | mmu-miR-125b-5p | MIMAT0000136 | 19898466 |
| Ectoderm | mmu-miR-128-3p | MIMAT0000140 | 19898466 |
| Embryoid Bodies | hsa-miR-302d-3p | MIMAT0000718 | 19229866 |
| Embryoid Bodies | hsa-miR-372-3p | MIMAT0000724 | 19229866 |
| Embryonal Carcinoma Stem | hsa-miR-125a-5p | MIMAT0000443 | 16227573 |
| Cells | | | |
| Embryonal Carcinoma Stem | hsa-miR-125b-5p | MIMAT0000423 | 16227573 |
| Cells | | | |
| Embryonal Carcinoma Stem | hsa-miR-200c-3p | MIMAT0000617 | 19665978 |
| Cells | | | |
| Embryonal Carcinoma Stem | hsa-miR-383-5p | MIMAT0000738 | 21368870 |
| Cells | | | |
| Embryonal Carcinoma Stem | mmu-miR-125b-5p | MIMAT0000136 | 16227573 |
| Cells | | | |
| Embryonic Stem Cells | hsa-miR-1 | MIMAT0000416 | 20081117 |
| Embryonic Stem Cells | hsa-miR-101-3p | MIMAT0000099 | 19818710 |
| Embryonic Stem Cells | hsa-miR-124-3p | MIMAT0000422 | 20362537 |
| Embryonic Stem Cells | hsa-miR-126-3p | MIMAT0000445 | 21304604 |
| Embryonic Stem Cells | hsa-miR-128-3p | MIMAT0000424 | 19409607 |
| Embryonic Stem Cells | hsa-miR-145-5p | MIMAT0000437 | 19409607 |
| Embryonic Stem Cells | hsa-miR-145-5p | MIMAT0000437 | 20382729 |
| Embryonic Stem Cells | hsa-miR-195-5p | MIMAT0000461 | 19823043 |
| Embryonic Stem Cells | hsa-miR-199a-5p | MIMAT0000231 | 19818710 |
| Embryonic Stem Cells | hsa-miR-203a | MIMAT0000264 | 18483491 |
| Embryonic Stem Cells | hsa-miR-214-3p | MIMAT0000271 | 19818710 |
| Embryonic Stem Cells | hsa-miR-26b-5p | MIMAT0000083 | 20831567 |
| Embryonic Stem Cells | hsa-miR-302a-3p | MIMAT0000684 | 21266536 |
| Embryonic Stem Cells | hsa-miR-302a-3p | MIMAT0000684 | 21062975 |
| Embryonic Stem Cells | hsa-miR-302a-3p | MIMAT0000684 | 19386261 |
| Embryonic Stem Cells | hsa-miR-302a-3p | MIMAT0000684 | 21151097 |
| Embryonic Stem Cells | hsa-miR-302a-3p | MIMAT0000684 | 23185040 |
| Embryonic Stem Cells | hsa-miR-302b-3p | MIMAT0000715 | 23185040 |
| Embryonic Stem Cells | hsa-miR-302b-3p | MIMAT0000715 | 21062975 |
| Embryonic Stem Cells | hsa-miR-302c-3p | MIMAT0000717 | 23185040 |
| Embryonic Stem Cells | hsa-miR-302c-3p | MIMAT0000717 | 21062975 |
| Embryonic Stem Cells | hsa-miR-302d-3p | MIMAT0000718 | 19229866 |
| Embryonic Stem Cells | hsa-miR-302d-3p | MIMAT0000718 | 23185040 |
| Embryonic Stem Cells | hsa-miR-302d-3p | MIMAT0000718 | 21266536 |
| Embryonic Stem Cells | hsa-miR-302d-3p | MIMAT0000718 | 21062975 |
| Embryonic Stem Cells | hsa-miR-372-3p | MIMAT0000724 | 19823043 |
| Embryonic Stem Cells | hsa-miR-372-3p | MIMAT0000724 | 19229866 |
| Embryonic Stem Cells | hsa-miR-375 | MIMAT0000728 | 19807270 |
| Embryonic Stem Cells | hsa-miR-380-5p | MIMAT0000734 | 20871609 |
| Embryonic Stem Cells | hsa-miR-499a-5p | MIMAT0002870 | 20081117 |
| Embryonic Stem Cells | hsa-miR-499b-5p | MIMAT0019897 | 20081117 |
| Embryonic Stem Cells | hsa-miR-519c-3p | MIMAT0002832 | 19825807 |
| Embryonic Stem Cells | hsa-miR-520h | MIMAT0002867 | 19825807 |
| Embryonic Stem Cells | hsa-miR-522-3p | MIMAT0002868 | 21304604 |
| Embryonic Stem Cells | hsa-miR-9-5p | MIMAT0000441 | 20362537 |
| Embryonic Stem Cells | hsa-miR-92b-3p | MIMAT0003218 | 19544458 |
| Embryonic Stem Cells | hsa-miR-98-5p | MIMAT0000096 | 19818710 |
| Embryonic Stem Cells | mmu-let-7c-5p | MIMAT0000523 | 20054295 |
| Embryonic Stem Cells | mmu-miR-100-3p | MIMAT0017051 | 20439489 |
| Embryonic Stem Cells | mmu-miR-100-5p | MIMAT0000655 | 20439489 |
| Embryonic Stem Cells | mmu-miR-10a-3p | MIMAT0004659 | 20118242 |
| Embryonic Stem Cells | mmu-miR-10a-5p | MIMAT0000648 | 20118242 |
| Embryonic Stem Cells | mmu-miR-134-5p | MIMAT0000146 | 17916804 |
| Embryonic Stem Cells | mmu-miR-134-5p | MIMAT0000146 | 18806776 |
| Embryonic Stem Cells | mmu-miR-137-3p | MIMAT0000149 | 20439489 |
| Embryonic Stem Cells | mmu-miR-137-5p | MIMAT0016986 | 20439489 |
| Embryonic Stem Cells | mmu-miR-1a-3p | MIMAT0000123 | 19521018 |
| Embryonic Stem Cells | mmu-miR-1a-3p | MIMAT0000123 | 20799856 |
| Embryonic Stem Cells | mmu-miR-214-3p | MIMAT0000661 | 19818710 |
| Embryonic Stem Cells | mmu-miR-290a-3p | MIMAT0004572 | 19628328 |
| Embryonic Stem Cells | mmu-miR-291a-3p | MIMAT0000368 | 19628328 |
| Embryonic Stem Cells | mmu-miR-292-3p | MIMAT0000370 | 19628328 |
| Embryonic Stem Cells | mmu-miR-293-3p | MIMAT0000371 | 19628328 |
| Embryonic Stem Cells | mmu-miR-294-3p | MIMAT0000372 | 19628328 |
| Embryonic Stem Cells | mmu-miR-295-3p | MIMAT0000373 | 19628328 |
| Embryonic Stem Cells | mmu-miR-296-5p | MIMAT0000374 | 18806776 |
| Embryonic Stem Cells | mmu-miR-466l-3p | MIMAT0005830 | 20410487 |
| Embryonic Stem Cells | mmu-miR-470-5p | MIMAT0002111 | 18806776 |
| Embryonic Stem Cells | mmu-miR-9-5p | MIMAT0000142 | 22465325 |
| Embryonic Stem Cells | mmu-miR-9-5p | MIMAT0000142 | 20362537 |
| Embryonic Structures | hsa-miR-122-5p | MIMAT0000421 | 20842632 |
| Embryonic Structures | hsa-miR-125b-5p | MIMAT0000423 | 19293287 |
| Embryonic Structures | hsa-miR-126-3p | MIMAT0000445 | 18694566 |
| Embryonic Structures | hsa-miR-155-5p | MIMAT0000646 | 19701459 |
| Embryonic Structures | hsa-miR-155-5p | MIMAT0000646 | 19759154 |
| Embryonic Structures | hsa-miR-196a-5p | MIMAT0000226 | 15105502 |
| Embryonic Structures | hsa-miR-19a-3p | MIMAT0000073 | 19296935 |
| Embryonic Structures | hsa-miR-21-5p | MIMAT0000076 | 20813833 |
| Embryonic Structures | hsa-miR-222-3p | MIMAT0000279 | 19589872 |
| Embryonic Structures | hsa-miR-24-3p | MIMAT0000080 | 19296935 |
| Embryonic Structures | hsa-miR-27a-3p | MIMAT0000084 | 19666532 |
| Embryonic Structures | hsa-miR-27a-3p | MIMAT0000084 | 19114653 |
| Embryonic Structures | hsa-miR-27b-3p | MIMAT0000419 | 19666532 |
| Embryonic Structures | hsa-miR-302a-3p | MIMAT0000684 | 19386261 |
| Embryonic Structures | mmu-miR-101a-3p | MIMAT0000133 | 17848513 |
| Embryonic Structures | mmu-miR-130a-3p | MIMAT0000141 | 19582148 |
| Embryonic Structures | mmu-miR-133a-3p | MIMAT0000145 | 16380711 |
| Embryonic Structures | mmu-miR-135a-5p | MIMAT0000147 | 20619502 |
| Embryonic Structures | mmu-miR-137-3p | MIMAT0000149 | 21256124 |
| Embryonic Structures | mmu-miR-140-5p | MIMAT0000151 | 16828749 |
| Embryonic Structures | mmu-miR-153-3p | MIMAT0000163 | 21256124 |
| Embryonic Structures | mmu-miR-155-5p | MIMAT0000165 | 19701459 |
| Embryonic Structures | mmu-miR-16-5p | MIMAT0000527 | 20619502 |
| Embryonic Structures | mmu-miR-18a-5p | MIMAT0000528 | 19114653 |
| Embryonic Structures | mmu-miR-196a-5p | MIMAT0000518 | 15105502 |
| Embryonic Structures | mmu-miR-196a-5p | MIMAT0000518 | 15361871 |
| Embryonic Structures | mmu-miR-199a-3p | MIMAT0000230 | 17848513 |
| Embryonic Structures | mmu-miR-199a-3p | MIMAT0000230 | 19114653 |
| Embryonic Structures | mmu-miR-1a-3p | MIMAT0000123 | 16380711 |
| Embryonic Structures | mmu-miR-20a-5p | MIMAT0000529 | 20619502 |
| Embryonic Structures | mmu-miR-21a-5p | MIMAT0000530 | 18556655 |
| Embryonic Structures | mmu-miR-27a-3p | MIMAT0000537 | 19114653 |
| Embryonic Structures | mmu-miR-29b-3p | MIMAT0000127 | 19509302 |
| Embryonic Structures | mmu-miR-30c-5p | MIMAT0000514 | 19114653 |
| Embryonic Structures | mmu-miR-363-3p | MIMAT0000708 | 21256124 |
| Embryonic Structures | mmu-miR-9-5p | MIMAT0000142 | 19114653 |
| Endocrine Cells | hsa-miR-375 | MIMAT0000728 | 15538371 |
| Endocrine Cells | mmu-miR-375-3p | MIMAT0000739 | 15538371 |
| Endocrine System | hsa-miR-1 | MIMAT0000416 | 21169019 |
| Endocrine System | hsa-miR-29a-3p | MIMAT0000086 | 21169019 |
| Endoderm | hsa-miR-375 | MIMAT0000728 | 19807270 |
| Endometrium | hsa-miR-153-3p | MIMAT0000439 | 20028871 |
| Endometrium | hsa-miR-183-5p | MIMAT0000261 | 20028871 |
| Endometrium | hsa-miR-186-5p | MIMAT0000456 | 20028871 |
| Endometrium | hsa-miR-196b-5p | MIMAT0001080 | 23293219 |
| Endometrium | hsa-miR-27a-3p | MIMAT0000084 | 20028871 |
| Endometrium | hsa-miR-29c-3p | MIMAT0000681 | 21436257 |
| Endometrium | hsa-miR-9-5p | MIMAT0000441 | 20028871 |
| Endometrium | hsa-miR-96-5p | MIMAT0000095 | 20028871 |
| Endoplasmic Reticulum | hsa-miR-346 | MIMAT0000773 | 22002058 |
| Endosomes | hsa-let-7d-5p | MIMAT0000065 | 20410187 |
| Endothelial Cells | hsa-miR-10a-5p | MIMAT0000253 | 20624982 |
| Endothelial Cells | hsa-miR-10b-5p | MIMAT0000254 | 21642433 |
| Endothelial Cells | hsa-miR-122-5p | MIMAT0000421 | 19726678 |
| Endothelial Cells | hsa-miR-126-3p | MIMAT0000445 | 18227515 |
| Endothelial Cells | hsa-miR-126-3p | MIMAT0000445 | 18694566 |
| Endothelial Cells | hsa-miR-126-3p | MIMAT0000445 | 22867989 |
| Endothelial Cells | hsa-miR-130a-3p | MIMAT0000425 | 17957028 |
| Endothelial Cells | hsa-miR-146a-5p | MIMAT0000449 | 18174313 |
| Endothelial Cells | hsa-miR-146a-5p | MIMAT0000449 | 19944095 |
| Endothelial Cells | hsa-miR-155-5p | MIMAT0000646 | 23108656 |
| Endothelial Cells | hsa-miR-155-5p | MIMAT0000646 | 19783678 |
| Endothelial Cells | hsa-miR-155-5p | MIMAT0000646 | 21310411 |
| Endothelial Cells | hsa-miR-16-5p | MIMAT0000069 | 21885851 |
| Endothelial Cells | hsa-miR-17-5p | MIMAT0000070 | 20299512 |
| Endothelial Cells | hsa-miR-181a-5p | MIMAT0000256 | 20558617 |
| Endothelial Cells | hsa-miR-18a-5p | MIMAT0000072 | 20299512 |
| Endothelial Cells | hsa-miR-195-5p | MIMAT0000461 | 20952681 |
| Endothelial Cells | hsa-miR-199a-3p | MIMAT0000232 | 21807947 |
| Endothelial Cells | hsa-miR-199a-5p | MIMAT0000231 | 23760629 |
| Endothelial Cells | hsa-miR-199a-5p | MIMAT0000231 | 19783678 |
| Endothelial Cells | hsa-miR-19a-3p | MIMAT0000073 | 20133739 |
| Endothelial Cells | hsa-miR-19a-3p | MIMAT0000073 | 20299512 |
| Endothelial Cells | hsa-miR-20a-5p | MIMAT0000075 | 20299512 |
| Endothelial Cells | hsa-miR-21-5p | MIMAT0000076 | 23313253 |
| Endothelial Cells | hsa-miR-21-5p | MIMAT0000076 | 21347332 |
| Endothelial Cells | hsa-miR-21-5p | MIMAT0000076 | 23496142 |
| Endothelial Cells | hsa-miR-210-3p | MIMAT0000267 | 19826008 |
| Endothelial Cells | hsa-miR-210-3p | MIMAT0000267 | 23322395 |
| Endothelial Cells | hsa-miR-210-3p | MIMAT0000267 | 18417479 |
| Endothelial Cells | hsa-miR-210-3p | MIMAT0000267 | 19808020 |
| Endothelial Cells | hsa-miR-214-3p | MIMAT0000271 | 22227154 |
| Endothelial Cells | hsa-miR-216a-5p | MIMAT0000273 | 19786632 |
| Endothelial Cells | hsa-miR-216a-5p | MIMAT0000273 | 21149267 |
| Endothelial Cells | hsa-miR-217 | MIMAT0000274 | 19786632 |
| Endothelial Cells | hsa-miR-221-3p | MIMAT0000278 | 20516212 |
| Endothelial Cells | hsa-miR-222-3p | MIMAT0000279 | 20489169 |
| Endothelial Cells | hsa-miR-23b-3p | MIMAT0000418 | 20133741 |
| Endothelial Cells | hsa-miR-296-5p | MIMAT0000690 | 18977327 |
| Endothelial Cells | hsa-miR-29b-3p | MIMAT0000100 | 21793034 |
| Endothelial Cells | hsa-miR-31-5p | MIMAT0000089 | 23233723 |
| Endothelial Cells | hsa-miR-330-3p | MIMAT0000751 | 21149267 |
| Endothelial Cells | hsa-miR-34a-5p | MIMAT0000255 | 20627091 |
| Endothelial Cells | hsa-miR-409-3p | MIMAT0001639 | 22531314 |
| Endothelial Cells | hsa-miR-424-5p | MIMAT0001341 | 20972335 |
| Endothelial Cells | hsa-miR-424-5p | MIMAT0001341 | 21885851 |
| Endothelial Cells | hsa-miR-503-5p | MIMAT0002874 | 21220732 |
| Endothelial Cells | hsa-miR-519c-3p | MIMAT0002832 | 20233879 |
| Endothelial Cells | hsa-miR-608 | MIMAT0003276 | 21149267 |
| Endothelial Cells | hsa-miR-92a-3p | MIMAT0000092 | 19460962 |
| Endothelial Cells | hsa-miR-93-5p | MIMAT0000093 | 20956944 |
| Endothelial Cells | mmu-miR-100-3p | MIMAT0017051 | 21339483 |
| Endothelial Cells | mmu-miR-100-5p | MIMAT0000655 | 21339483 |
| Endothelial Cells | mmu-miR-126a-3p | MIMAT0000138 | 18694565 |
| Endothelial Cells | mmu-miR-15a-5p | MIMAT0000526 | 20445066 |
| Endothelial Cells | mmu-miR-29c-3p | MIMAT0000536 | 21310958 |
| Endothelial Cells | mmu-miR-34a-5p | MIMAT0000542 | 20424141 |
| Endothelial Cells | rno-miR-320-3p | MIMAT0000903 | 18986336 |
| Endothelium | hsa-miR-10a-5p | MIMAT0000253 | 20624982 |
| Endothelium | hsa-miR-424-5p | MIMAT0001341 | 20972335 |
| Enterocytes | hsa-miR-510-5p | MIMAT0002882 | 18614545 |
| Enterocytes | hsa-miR-626 | MIMAT0003295 | 19892711 |
| Enterocytes | hsa-miR-7-5p | MIMAT0000252 | 19892711 |
| Enterocytes | mmu-miR-706 | MIMAT0003496 | 19892711 |
| Epidermis | hsa-miR-146a-5p | MIMAT0000449 | 20375304 |
| Epidermis | hsa-miR-203a | MIMAT0000264 | 18483491 |
| Epidermis | hsa-miR-31-5p | MIMAT0000089 | 23233723 |
| Epidermis | mmu-let-7a-5p | MIMAT0000521 | 19898466 |
| Epidermis | mmu-let-7e-5p | MIMAT0000524 | 19898466 |
| Epidermis | mmu-miR-125a-5p | MIMAT0000135 | 19898466 |
| Epidermis | mmu-miR-125b-5p | MIMAT0000136 | 19898466 |
| Epidermis | mmu-miR-128-3p | MIMAT0000140 | 19898466 |
| Epididymis | rno-miR-200c-3p | MIMAT0000873 | 20705680 |
| Epididymis | rno-miR-335 | MIMAT0000575 | 20933506 |
| Epithelial Cells | hsa-let-7d-5p | MIMAT0000065 | 20395557 |
| Epithelial Cells | hsa-miR-106a-5p | MIMAT0000103 | 20889678 |
| Epithelial Cells | hsa-miR-106b-5p | MIMAT0000680 | 20101223 |
| Epithelial Cells | hsa-miR-1226-3p | MIMAT0005577 | 20514397 |
| Epithelial Cells | hsa-miR-1258 | MIMAT0005909 | 21266359 |
| Epithelial Cells | hsa-miR-126-3p | MIMAT0000445 | 20083669 |
| Epithelial Cells | hsa-miR-128-3p | MIMAT0000424 | 20388705 |
| Epithelial Cells | hsa-miR-133a-3p | MIMAT0000427 | 18464261 |
| Epithelial Cells | hsa-miR-133b | MIMAT0000770 | 18464261 |
| Epithelial Cells | hsa-miR-141-3p | MIMAT0000432 | 19854497 |
| Epithelial Cells | hsa-miR-141-3p | MIMAT0000432 | 18835392 |
| Epithelial Cells | hsa-miR-141-3p | MIMAT0000432 | 18411277 |
| Epithelial Cells | hsa-miR-146a-5p | MIMAT0000449 | 20101223 |
| Epithelial Cells | hsa-miR-146a-5p | MIMAT0000449 | 20124483 |
| Epithelial Cells | hsa-miR-146a-5p | MIMAT0000449 | 20542134 |
| Epithelial Cells | hsa-miR-146b-5p | MIMAT0002809 | 20101223 |
| Epithelial Cells | hsa-miR-155-5p | MIMAT0000646 | 18794355 |
| Epithelial Cells | hsa-miR-15a-5p | MIMAT0000068 | 18949056 |
| Epithelial Cells | hsa-miR-184 | MIMAT0000454 | 19033458 |
| Epithelial Cells | hsa-miR-192-5p | MIMAT0000222 | 18835392 |
| Epithelial Cells | hsa-miR-197-3p | MIMAT0000227 | 19671678 |
| Epithelial Cells | hsa-miR-200a-3p | MIMAT0000682 | 18829540 |
| Epithelial Cells | hsa-miR-200a-3p | MIMAT0000682 | 18411277 |
| Epithelial Cells | hsa-miR-200a-3p | MIMAT0000682 | 18835392 |
| Epithelial Cells | hsa-miR-200a-3p | MIMAT0000682 | 19854497 |
| Epithelial Cells | hsa-miR-200b-3p | MIMAT0000318 | 18829540 |
| Epithelial Cells | hsa-miR-200b-3p | MIMAT0000318 | 18411277 |
| Epithelial Cells | hsa-miR-200b-3p | MIMAT0000318 | 19854497 |
| Epithelial Cells | hsa-miR-200c-3p | MIMAT0000617 | 18411277 |
| Epithelial Cells | hsa-miR-200c-3p | MIMAT0000617 | 19435871 |
| Epithelial Cells | hsa-miR-200c-3p | MIMAT0000617 | 19854497 |
| Epithelial Cells | hsa-miR-203a | MIMAT0000264 | 18483491 |
| Epithelial Cells | hsa-miR-203a | MIMAT0000264 | 22101077 |
| Epithelial Cells | hsa-miR-205-5p | MIMAT0000266 | 19033458 |
| Epithelial Cells | hsa-miR-21-5p | MIMAT0000076 | 23292313 |
| Epithelial Cells | hsa-miR-21-5p | MIMAT0000076 | 19473551 |
| Epithelial Cells | hsa-miR-21-5p | MIMAT0000076 | 21047769 |
| Epithelial Cells | hsa-miR-21-5p | MIMAT0000076 | 19435867 |
| Epithelial Cells | hsa-miR-212-3p | MIMAT0000269 | 18162065 |
| Epithelial Cells | hsa-miR-217 | MIMAT0000274 | 18835392 |
| Epithelial Cells | hsa-miR-218-5p | MIMAT0000275 | 19168627 |
| Epithelial Cells | hsa-miR-221-3p | MIMAT0000278 | 21236259 |
| Epithelial Cells | hsa-miR-27b-3p | MIMAT0000419 | 18835392 |
| Epithelial Cells | hsa-miR-27b-3p | MIMAT0000419 | 20388705 |
| Epithelial Cells | hsa-miR-302a-3p | MIMAT0000684 | 20101223 |
| Epithelial Cells | hsa-miR-330-3p | MIMAT0000751 | 19597470 |
| Epithelial Cells | hsa-miR-346 | MIMAT0000773 | 22002058 |
| Epithelial Cells | hsa-miR-429 | MIMAT0001536 | 18411277 |
| Epithelial Cells | hsa-miR-429 | MIMAT0001536 | 19854497 |
| Epithelial Cells | hsa-miR-429 | MIMAT0001536 | 18829540 |
| Epithelial Cells | hsa-miR-451a | MIMAT0001631 | 19318487 |
| Epithelial Cells | hsa-miR-483-3p | MIMAT0002173 | 22101077 |
| Epithelial Cells | hsa-miR-497-5p | MIMAT0002820 | 23453369 |
| Epithelial Cells | hsa-miR-513a-5p | MIMAT0002877 | 19155478 |
| Epithelial Cells | hsa-miR-532-5p | MIMAT0002888 | 18835392 |
| Epithelial Cells | hsa-miR-595 | MIMAT0003263 | 22101077 |
| Epithelial Cells | hsa-miR-603 | MIMAT0003271 | 18835392 |
| Epithelial Cells | hsa-miR-622 | MIMAT0003291 | 22016468 |
| Epithelial Cells | hsa-miR-7-5p | MIMAT0000252 | 19033458 |
| Epithelial Cells | hsa-miR-769-5p | MIMAT0003886 | 18835392 |
| Epithelial Cells | hsa-miR-92b-3p | MIMAT0003218 | 21030610 |
| Epithelial Cells | hsa-miR-93-5p | MIMAT0000093 | 19671678 |
| Epithelial Cells | hsa-miR-96-5p | MIMAT0000095 | 21203424 |
| Epithelial Cells | hsa-miR-98-5p | MIMAT0000096 | 19671678 |
| Epithelial Cells | hsa-miR-98-5p | MIMAT0000096 | 20486857 |
| Epithelial Cells | mmu-miR-128-3p | MIMAT0000140 | 20388705 |
| Epithelial Cells | mmu-miR-141-3p | MIMAT0000153 | 18411277 |
| Epithelial Cells | mmu-miR-155-5p | MIMAT0000165 | 21807897 |
| Epithelial Cells | mmu-miR-155-5p | MIMAT0000165 | 18794355 |
| Epithelial Cells | mmu-miR-17-5p | MIMAT0000649 | 17765889 |
| Epithelial Cells | mmu-miR-200a-3p | MIMAT0000519 | 18411277 |
| Epithelial Cells | mmu-miR-200b-3p | MIMAT0000233 | 18411277 |
| Epithelial Cells | mmu-miR-27b-3p | MIMAT0000126 | 20388705 |
| Epithelial Cells | mmu-miR-429-3p | MIMAT0001537 | 18411277 |
| Epithelium | hsa-miR-101-3p | MIMAT0000099 | 19043531 |
| Epithelium | hsa-miR-125b-5p | MIMAT0000423 | 19378336 |
| Epithelium | hsa-miR-133a-3p | MIMAT0000427 | 19378336 |
| Epithelium | hsa-miR-141-3p | MIMAT0000432 | 20053927 |
| Epithelium | hsa-miR-143-3p | MIMAT0000435 | 19157460 |
| Epithelium | hsa-miR-145-5p | MIMAT0000437 | 19378336 |
| Epithelium | hsa-miR-146a-5p | MIMAT0000449 | 18174313 |
| Epithelium | hsa-miR-155-5p | MIMAT0000646 | 19701459 |
| Epithelium | hsa-miR-182-5p | MIMAT0000259 | 17597072 |
| Epithelium | hsa-miR-195-5p | MIMAT0000461 | 19378336 |
| Epithelium | hsa-miR-199a-3p | MIMAT0000232 | 19378336 |
| Epithelium | hsa-miR-203a | MIMAT0000264 | 18483491 |
| Epithelium | hsa-miR-204-5p | MIMAT0000265 | 20056717 |
| Epithelium | hsa-miR-21-5p | MIMAT0000076 | 20346171 |
| Epithelium | hsa-miR-21-5p | MIMAT0000076 | 19473551 |
| Epithelium | hsa-miR-21-5p | MIMAT0000076 | 20056717 |
| Epithelium | hsa-miR-218-5p | MIMAT0000275 | 19890957 |
| Epithelium | hsa-miR-218-5p | MIMAT0000275 | 19168627 |
| Epithelium | hsa-miR-29a-3p | MIMAT0000086 | 18390668 |
| Epithelium | hsa-miR-29c-3p | MIMAT0000681 | 18390668 |
| Epithelium | hsa-miR-30a-3p | MIMAT0000088 | 19378336 |
| Epithelium | hsa-miR-30a-5p | MIMAT0000087 | 19378336 |
| Epithelium | hsa-miR-510-5p | MIMAT0002882 | 18614545 |
| Epithelium | hsa-miR-96-5p | MIMAT0000095 | 17597072 |
| Epithelium | mmu-miR-106b-5p | MIMAT0000386 | 19559694 |
| Epithelium | mmu-miR-155-5p | MIMAT0000165 | 19701459 |
| Epithelium | mmu-miR-17-5p | MIMAT0000649 | 17765889 |
| Epithelium | mmu-miR-17-5p | MIMAT0000649 | 19559694 |
| Epithelium | mmu-miR-20a-5p | MIMAT0000529 | 19559694 |
| Epithelium | mmu-miR-21a-5p | MIMAT0000530 | 21295561 |
| Erythroblasts | hsa-miR-223-3p | MIMAT0000280 | 20826802 |
| Erythroblasts | hsa-miR-223-3p | MIMAT0000280 | 19278969 |
| Erythroblasts | hsa-miR-25-3p | MIMAT0000081 | 20826802 |
| Erythroblasts | mmu-miR-223-3p | MIMAT0000665 | 20826802 |
| Erythroblasts | mmu-miR-25-3p | MIMAT0000652 | 20826802 |
| Erythrocytes | hsa-let-7a-5p | MIMAT0000062 | 19939273 |
| Erythrocytes | hsa-let-7c-3p | MIMAT0026472 | 19939273 |
| Erythrocytes | hsa-let-7c-5p | MIMAT0000064 | 19939273 |
| Erythrocytes | hsa-let-7d-3p | MIMAT0004484 | 19939273 |
| Erythrocytes | hsa-let-7d-5p | MIMAT0000065 | 19939273 |
| Erythrocytes | hsa-let-7e-3p | MIMAT0004485 | 19939273 |
| Erythrocytes | hsa-let-7e-5p | MIMAT0000066 | 19939273 |
| Erythrocytes | hsa-let-7g-3p | MIMAT0004584 | 19939273 |
| Erythrocytes | hsa-let-7g-5p | MIMAT0000414 | 19939273 |
| Erythrocytes | hsa-let-7i-3p | MIMAT0004585 | 19939273 |
| Erythrocytes | hsa-let-7i-5p | MIMAT0000415 | 19939273 |
| Erythrocytes | hsa-miR-320a | MIMAT0000510 | 18523662 |
| Erythroid Cells | hsa-let-7a-5p | MIMAT0000062 | 19939273 |
| Erythroid Cells | hsa-let-7c-3p | MIMAT0026472 | 19939273 |
| Erythroid Cells | hsa-let-7c-5p | MIMAT0000064 | 19939273 |
| Erythroid Cells | hsa-let-7d-3p | MIMAT0004484 | 19939273 |
| Erythroid Cells | hsa-let-7d-5p | MIMAT0000065 | 19939273 |
| Erythroid Cells | hsa-let-7d-5p | MIMAT0000065 | 20410187 |
| Erythroid Cells | hsa-let-7e-3p | MIMAT0004485 | 19939273 |
| Erythroid Cells | hsa-let-7e-5p | MIMAT0000066 | 19939273 |
| Erythroid Cells | hsa-let-7g-3p | MIMAT0004584 | 19939273 |
| Erythroid Cells | hsa-let-7g-5p | MIMAT0000414 | 19939273 |
| Erythroid Cells | hsa-let-7i-3p | MIMAT0004585 | 19939273 |
| Erythroid Cells | hsa-let-7i-5p | MIMAT0000415 | 19939273 |
| Erythroid Cells | hsa-miR-150-5p | MIMAT0000451 | 18539114 |
| Erythroid Cells | hsa-miR-223-3p | MIMAT0000280 | 19278969 |
| Erythroid Cells | hsa-miR-223-3p | MIMAT0000280 | 19017354 |
| Erythroid Precursor Cells | hsa-miR-15a-5p | MIMAT0000068 | 21205891 |
| Erythroid Precursor Cells | hsa-miR-16-5p | MIMAT0000069 | 21205891 |
| Esophagus | hsa-miR-196a-5p | MIMAT0000226 | 19342367 |
| Esophagus | hsa-miR-21-5p | MIMAT0000076 | 18372920 |
| Esophagus | mmu-miR-21a-5p | MIMAT0000530 | 18372920 |
| Eukaryotic Cells | hsa-let-7a-5p | MIMAT0000062 | 19939273 |
| Eukaryotic Cells | hsa-let-7c-3p | MIMAT0026472 | 19939273 |
| Eukaryotic Cells | hsa-let-7c-5p | MIMAT0000064 | 19939273 |
| Eukaryotic Cells | hsa-let-7d-3p | MIMAT0004484 | 19939273 |
| Eukaryotic Cells | hsa-let-7d-5p | MIMAT0000065 | 19939273 |
| Eukaryotic Cells | hsa-let-7e-3p | MIMAT0004485 | 19939273 |
| Eukaryotic Cells | hsa-let-7e-5p | MIMAT0000066 | 19939273 |
| Eukaryotic Cells | hsa-let-7g-3p | MIMAT0004584 | 19939273 |
| Eukaryotic Cells | hsa-let-7g-5p | MIMAT0000414 | 19939273 |
| Eukaryotic Cells | hsa-let-7i-3p | MIMAT0004585 | 19939273 |
| Eukaryotic Cells | hsa-let-7i-5p | MIMAT0000415 | 19939273 |
| Extracellular Matrix | bta-miR-335 | MIMAT0009291 | 18185580 |
| Extracellular Matrix | cfa-miR-335 | MIMAT0006624 | 18185580 |
| Extracellular Matrix | hsa-miR-124-3p | MIMAT0000422 | 21112327 |
| Extracellular Matrix | hsa-miR-125b-5p | MIMAT0000423 | 21703189 |
| Extracellular Matrix | hsa-miR-155-5p | MIMAT0000646 | 19701459 |
| Extracellular Matrix | hsa-miR-17-3p | MIMAT0000071 | 19771525 |
| Extracellular Matrix | hsa-miR-17-5p | MIMAT0000070 | 19771525 |
| Extracellular Matrix | hsa-miR-205-5p | MIMAT0000266 | 22578566 |
| Extracellular Matrix | hsa-miR-25-3p | MIMAT0000081 | 19541842 |
| Extracellular Matrix | hsa-miR-29a-3p | MIMAT0000086 | 18390668 |
| Extracellular Matrix | hsa-miR-29a-3p | MIMAT0000086 | 21665270 |
| Extracellular Matrix | hsa-miR-29b-3p | MIMAT0000100 | 21665270 |
| Extracellular Matrix | hsa-miR-29b-3p | MIMAT0000100 | 19956414 |
| Extracellular Matrix | hsa-miR-29b-3p | MIMAT0000100 | 21793034 |
| Extracellular Matrix | hsa-miR-29c-3p | MIMAT0000681 | 18390668 |
| Extracellular Matrix | hsa-miR-29c-3p | MIMAT0000681 | 21436257 |
| Extracellular Matrix | hsa-miR-335-5p | MIMAT0000765 | 18185580 |
| Extracellular Matrix | hsa-miR-378a-3p | MIMAT0000732 | 19844573 |
| Extracellular Matrix | hsa-miR-626 | MIMAT0003295 | 19892711 |
| Extracellular Matrix | hsa-miR-7-5p | MIMAT0000252 | 19892711 |
| Extracellular Matrix | mmu-miR-125b-5p | MIMAT0000136 | 21945074 |
| Extracellular Matrix | mmu-miR-143-3p | MIMAT0000247 | 20489207 |
| Extracellular Matrix | mmu-miR-155-5p | MIMAT0000165 | 19701459 |
| Extracellular Matrix | mmu-miR-15a-5p | MIMAT0000526 | 21305018 |
| Extracellular Matrix | mmu-miR-192-5p | MIMAT0000517 | 17360662 |
| Extracellular Matrix | mmu-miR-215-5p | MIMAT0000904 | 17360662 |
| Extracellular Matrix | mmu-miR-216a-5p | MIMAT0000662 | 20713358 |
| Extracellular Matrix | mmu-miR-21a-5p | MIMAT0000530 | 21295561 |
| Extracellular Matrix | mmu-miR-27b-3p | MIMAT0000126 | 19342382 |
| Extracellular Matrix | mmu-miR-29a-3p | MIMAT0000535 | 21665270 |
| Extracellular Matrix | mmu-miR-29b-3p | MIMAT0000127 | 19342382 |
| Extracellular Matrix | mmu-miR-29b-3p | MIMAT0000127 | 21665270 |
| Extracellular Matrix | mmu-miR-29c-3p | MIMAT0000536 | 21310958 |
| Extracellular Matrix | mmu-miR-30a-5p | MIMAT0000128 | 21945074 |
| Extracellular Matrix | mmu-miR-30c-5p | MIMAT0000514 | 21945074 |
| Extracellular Matrix | mmu-miR-497-5p | MIMAT0003453 | 21305018 |
| Extracellular Matrix | mmu-miR-706 | MIMAT0003496 | 19892711 |
| Extracellular Matrix | ppy-miR-335 | MIMAT0015837 | 18185580 |
| Extracellular Matrix | ptr-miR-335 | MIMAT0008104 | 18185580 |
| Extracellular Matrix | rno-miR-150-5p | MIMAT0000853 | 19892940 |
| Extracellular Matrix | rno-miR-192-5p | MIMAT0000867 | 20393144 |
| Extracellular Matrix | rno-miR-194-5p | MIMAT0000869 | 19892940 |
| Extracellular Matrix | rno-miR-215 | MIMAT0003118 | 20393144 |
| Extracellular Matrix | rno-miR-335 | MIMAT0000575 | 18185580 |
| Extracellular Matrix | ssc-miR-335 | MIMAT0013955 | 18185580 |
| Extremities | hsa-miR-433-3p | MIMAT0001627 | 20181727 |
| Extremities | mmu-miR-137-3p | MIMAT0000149 | 21256124 |
| Extremities | mmu-miR-140-5p | MIMAT0000151 | 21872590 |
| Extremities | mmu-miR-140-5p | MIMAT0000151 | 16828749 |
| Extremities | mmu-miR-153-3p | MIMAT0000163 | 21256124 |
| Extremities | mmu-miR-363-3p | MIMAT0000708 | 21256124 |
| Extremities | rno-miR-138-5p | MIMAT0000844 | 20064393 |
| Eye | hsa-miR-1 | MIMAT0000416 | 21169019 |
| Eye | hsa-miR-204-5p | MIMAT0000265 | 20713703 |
| Eye | hsa-miR-29a-3p | MIMAT0000086 | 21169019 |
| Eye | mmu-miR-184-3p | MIMAT0000213 | 20795863 |
| Fallopian Tubes | hsa-miR-182-5p | MIMAT0000259 | 22322863 |
| Femoral Artery | mmu-miR-100-3p | MIMAT0017051 | 21339483 |
| Femoral Artery | mmu-miR-100-5p | MIMAT0000655 | 21339483 |
| Fetal Blood | hsa-let-7a-5p | MIMAT0000062 | 19939273 |
| Fetal Blood | hsa-let-7c-3p | MIMAT0026472 | 19939273 |
| Fetal Blood | hsa-let-7c-5p | MIMAT0000064 | 19939273 |
| Fetal Blood | hsa-let-7d-3p | MIMAT0004484 | 19939273 |
| Fetal Blood | hsa-let-7d-5p | MIMAT0000065 | 19939273 |
| Fetal Blood | hsa-let-7e-3p | MIMAT0004485 | 19939273 |
| Fetal Blood | hsa-let-7e-5p | MIMAT0000066 | 19939273 |
| Fetal Blood | hsa-let-7g-3p | MIMAT0004584 | 19939273 |
| Fetal Blood | hsa-let-7g-5p | MIMAT0000414 | 19939273 |
| Fetal Blood | hsa-let-7i-3p | MIMAT0004585 | 19939273 |
| Fetal Blood | hsa-let-7i-5p | MIMAT0000415 | 19939273 |
| Fetal Blood | hsa-miR-129-5p | MIMAT0000242 | 18189265 |
| Fetal Blood | hsa-miR-135b-5p | MIMAT0000758 | 19795981 |
| Fetal Blood | hsa-miR-148a-3p | MIMAT0000243 | 19435428 |
| Fetal Blood | hsa-miR-155-5p | MIMAT0000646 | 18950466 |
| Fetal Blood | hsa-miR-184 | MIMAT0000454 | 19286996 |
| Fetal Blood | hsa-miR-19b-3p | MIMAT0000074 | 19435428 |
| Fetal Blood | hsa-miR-20a-5p | MIMAT0000075 | 19435428 |
| Fetal Blood | hsa-miR-214-3p | MIMAT0000271 | 19435428 |
| Fetal Blood | hsa-miR-223-3p | MIMAT0000280 | 19278969 |
| Fetal Blood | hsa-miR-31-3p | MIMAT0004504 | 19408243 |
| Fetal Blood | hsa-miR-31-5p | MIMAT0000089 | 19408243 |
| Fetal Blood | hsa-miR-520h | MIMAT0002867 | 18189265 |
| Fetal Blood | hsa-miR-520h | MIMAT0002867 | 19435428 |
| Fetal Blood | hsa-miR-92a-3p | MIMAT0000092 | 19435428 |
| Fetus | hsa-miR-433-3p | MIMAT0001627 | 20181727 |
| Fibroblasts | bta-miR-155 | MIMAT0009241 | 20735984 |
| Fibroblasts | cfa-miR-155 | MIMAT0006671 | 20735984 |
| Fibroblasts | dre-miR-155 | MIMAT0001851 | 20735984 |
| Fibroblasts | gga-miR-155 | MIMAT0001106 | 20735984 |
| Fibroblasts | hsa-let-7a-5p | MIMAT0000062 | 20005451 |
| Fibroblasts | hsa-let-7b-3p | MIMAT0004482 | 19126550 |
| Fibroblasts | hsa-let-7b-5p | MIMAT0000063 | 19126550 |
| Fibroblasts | hsa-miR-125b-5p | MIMAT0000423 | 21412257 |
| Fibroblasts | hsa-miR-125b-5p | MIMAT0000423 | 19293287 |
| Fibroblasts | hsa-miR-140-5p | MIMAT0000431 | 23401231 |
| Fibroblasts | hsa-miR-141-3p | MIMAT0000432 | 19861690 |
| Fibroblasts | hsa-miR-146a-5p | MIMAT0000449 | 20061417 |
| Fibroblasts | hsa-miR-146a-5p | MIMAT0000449 | 18759964 |
| Fibroblasts | hsa-miR-146a-5p | MIMAT0000449 | 21968601 |
| Fibroblasts | hsa-miR-146b-5p | MIMAT0002809 | 21305051 |
| Fibroblasts | hsa-miR-155-5p | MIMAT0000646 | 21093163 |
| Fibroblasts | hsa-miR-155-5p | MIMAT0000646 | 20735984 |
| Fibroblasts | hsa-miR-155-5p | MIMAT0000646 | 19701459 |
| Fibroblasts | hsa-miR-155-5p | MIMAT0000646 | 16675453 |
| Fibroblasts | hsa-miR-155-5p | MIMAT0000646 | 19759154 |
| Fibroblasts | hsa-miR-155-5p | MIMAT0000646 | 17668390 |
| Fibroblasts | hsa-miR-15a-5p | MIMAT0000068 | 19823025 |
| Fibroblasts | hsa-miR-15b-5p | MIMAT0000417 | 19861690 |
| Fibroblasts | hsa-miR-16-5p | MIMAT0000069 | 21885851 |
| Fibroblasts | hsa-miR-17-5p | MIMAT0000070 | 19823025 |
| Fibroblasts | hsa-miR-183-5p | MIMAT0000261 | 19940135 |
| Fibroblasts | hsa-miR-199a-3p | MIMAT0000232 | 18456660 |
| Fibroblasts | hsa-miR-206 | MIMAT0000462 | 17030984 |
| Fibroblasts | hsa-miR-20a-5p | MIMAT0000075 | 19823025 |
| Fibroblasts | hsa-miR-21-5p | MIMAT0000076 | 21826097 |
| Fibroblasts | hsa-miR-21-5p | MIMAT0000076 | 19509156 |
| Fibroblasts | hsa-miR-21-5p | MIMAT0000076 | 19435867 |
| Fibroblasts | hsa-miR-21-5p | MIMAT0000076 | 20813833 |
| Fibroblasts | hsa-miR-22-3p | MIMAT0000077 | 22538858 |
| Fibroblasts | hsa-miR-223-3p | MIMAT0000280 | 21305051 |
| Fibroblasts | hsa-miR-223-3p | MIMAT0000280 | 20826802 |
| Fibroblasts | hsa-miR-23a-3p | MIMAT0000078 | 21750350 |
| Fibroblasts | hsa-miR-23b-3p | MIMAT0000418 | 19490101 |
| Fibroblasts | hsa-miR-24-3p | MIMAT0000080 | 19861690 |
| Fibroblasts | hsa-miR-24-3p | MIMAT0000080 | 18365017 |
| Fibroblasts | hsa-miR-25-3p | MIMAT0000081 | 19861690 |
| Fibroblasts | hsa-miR-25-3p | MIMAT0000081 | 20826802 |
| Fibroblasts | hsa-miR-29a-3p | MIMAT0000086 | 20201077 |
| Fibroblasts | hsa-miR-29b-3p | MIMAT0000100 | 20201077 |
| Fibroblasts | hsa-miR-29b-3p | MIMAT0000100 | 21305051 |
| Fibroblasts | hsa-miR-29c-3p | MIMAT0000681 | 21436257 |
| Fibroblasts | hsa-miR-29c-3p | MIMAT0000681 | 21305051 |
| Fibroblasts | hsa-miR-31-3p | MIMAT0004504 | 20980827 |
| Fibroblasts | hsa-miR-31-5p | MIMAT0000089 | 20980827 |
| Fibroblasts | hsa-miR-335-5p | MIMAT0000765 | 21164520 |
| Fibroblasts | hsa-miR-378a-3p | MIMAT0000732 | 21471220 |
| Fibroblasts | hsa-miR-424-5p | MIMAT0001341 | 21885851 |
| Fibroblasts | hsa-miR-433-3p | MIMAT0001627 | 20181727 |
| Fibroblasts | hsa-miR-483-3p | MIMAT0002173 | 21305051 |
| Fibroblasts | hsa-miR-505-3p | MIMAT0002876 | 20923760 |
| Fibroblasts | hsa-miR-574-5p | MIMAT0004795 | 21305051 |
| Fibroblasts | mml-miR-155 | MIMAT0006216 | 20735984 |
| Fibroblasts | mmu-miR-101b-3p | MIMAT0000616 | 19169275 |
| Fibroblasts | mmu-miR-130a-3p | MIMAT0000141 | 19582148 |
| Fibroblasts | mmu-miR-133a-3p | MIMAT0000145 | 19043405 |
| Fibroblasts | mmu-miR-135b-5p | MIMAT0000612 | 23440414 |
| Fibroblasts | mmu-miR-143-3p | MIMAT0000247 | 19578358 |
| Fibroblasts | mmu-miR-145a-5p | MIMAT0000157 | 19578358 |
| Fibroblasts | mmu-miR-155-5p | MIMAT0000165 | 19701459 |
| Fibroblasts | mmu-miR-196a-5p | MIMAT0000518 | 19169275 |
| Fibroblasts | mmu-miR-200a-3p | MIMAT0000519 | 21115742 |
| Fibroblasts | mmu-miR-200b-3p | MIMAT0000233 | 21115742 |
| Fibroblasts | mmu-miR-200c-3p | MIMAT0000657 | 21115742 |
| Fibroblasts | mmu-miR-206-3p | MIMAT0000239 | 17030984 |
| Fibroblasts | mmu-miR-206-3p | MIMAT0000239 | 19043405 |
| Fibroblasts | mmu-miR-21a-5p | MIMAT0000530 | 19043405 |
| Fibroblasts | mmu-miR-21a-5p | MIMAT0000530 | 19147652 |
| Fibroblasts | mmu-miR-223-3p | MIMAT0000665 | 20826802 |
| Fibroblasts | mmu-miR-25-3p | MIMAT0000652 | 20826802 |
| Fibroblasts | mmu-miR-28a-3p | MIMAT0004661 | 20923760 |
| Fibroblasts | mmu-miR-28a-5p | MIMAT0000653 | 20923760 |
| Fibroblasts | mmu-miR-429-3p | MIMAT0001537 | 21115742 |
| Fibroblasts | mmu-miR-505-3p | MIMAT0003513 | 20923760 |
| Fibroblasts | mmu-miR-505-5p | MIMAT0017259 | 20923760 |
| Fibroblasts | ppy-miR-155 | MIMAT0015777 | 20735984 |
| Fibroblasts | ptr-miR-155 | MIMAT0008048 | 20735984 |
| Fibroblasts | rno-miR-21-5p | MIMAT0000790 | 22565856 |
| Fibroblasts | xtr-miR-155 | MIMAT0003608 | 20735984 |
| Focal Adhesions | hsa-miR-10b-5p | MIMAT0000254 | 22573479 |
| Focal Adhesions | hsa-miR-126-3p | MIMAT0000445 | 19767772 |
| Focal Adhesions | hsa-miR-151a-5p | MIMAT0004697 | 20305651 |
| Focal Adhesions | hsa-miR-21-5p | MIMAT0000076 | 17681183 |
| Fourth Ventricle | mmu-miR-449a-5p | MIMAT0001542 | 19056356 |
| Ganglia | hsa-miR-18a-5p | MIMAT0000072 | 20080637 |
| Ganglia | hsa-miR-19a-3p | MIMAT0000073 | 20080637 |
| Ganglia Spinal | hsa-miR-145-5p | MIMAT0000437 | 21276775 |
| Ganglia Spinal | hsa-miR-214-3p | MIMAT0000271 | 21276775 |
| Ganglia Sympathetic | hsa-miR-18a-5p | MIMAT0000072 | 20080637 |
| Ganglia Sympathetic | hsa-miR-19a-3p | MIMAT0000073 | 20080637 |
| Gap Junctions | hsa-miR-21-5p | MIMAT0000076 | 18508928 |
| Gap Junctions | mmu-miR-206-3p | MIMAT0000239 | 19933329 |
| Gap Junctions | mmu-miR-21a-5p | MIMAT0000530 | 18508928 |
| Gastric Mucosa | hsa-miR-146a-5p | MIMAT0000449 | 20542134 |
| Gastric Mucosa | hsa-miR-155-5p | MIMAT0000646 | 19650740 |
| Gastric Mucosa | hsa-miR-218-5p | MIMAT0000275 | 19890957 |
| Gastric Mucosa | hsa-miR-222-3p | MIMAT0000279 | 22321642 |
| Gastric Mucosa | hsa-miR-223-3p | MIMAT0000280 | 22270966 |
| Gastric Mucosa | hsa-miR-34b-3p | MIMAT0004676 | 20924086 |
| Gastric Mucosa | hsa-miR-34c-5p | MIMAT0000686 | 20924086 |
| Gastrointestinal Tract | hsa-miR-17-5p | MIMAT0000070 | 20926598 |
| Gastrointestinal Tract | hsa-miR-20a-5p | MIMAT0000075 | 20926598 |
| Gastrointestinal Tract | hsa-miR-20b-5p | MIMAT0001413 | 20926598 |
| Gastrointestinal Tract | hsa-miR-30c-5p | MIMAT0000244 | 20926598 |
| Gastrointestinal Tract | hsa-miR-30e-5p | MIMAT0000692 | 20926598 |
| Germ Cells | hsa-miR-1 | MIMAT0000416 | 18155131 |
| Germ Cells | hsa-miR-206 | MIMAT0000462 | 18155131 |
| Germ Cells | hsa-miR-221-3p | MIMAT0000278 | 18155131 |
| Germ Cells | hsa-miR-302b-3p | MIMAT0000715 | 19342891 |
| Germ Cells | hsa-miR-372-3p | MIMAT0000724 | 16564011 |
| Germ Cells | hsa-miR-372-3p | MIMAT0000724 | 18155131 |
| Germ Cells | hsa-miR-373-3p | MIMAT0000726 | 16564011 |
| Germ Cells | hsa-miR-383-5p | MIMAT0000738 | 21368870 |
| Germ Cells | mmu-let-7a-5p | MIMAT0000521 | 19898466 |
| Germ Cells | mmu-let-7e-5p | MIMAT0000524 | 19898466 |
| Germ Cells | mmu-miR-125a-5p | MIMAT0000135 | 19898466 |
| Germ Cells | mmu-miR-125b-5p | MIMAT0000136 | 19898466 |
| Germ Cells | mmu-miR-128-3p | MIMAT0000140 | 19898466 |
| Germ Cells | mmu-miR-290a-3p | MIMAT0004572 | 19628328 |
| Germ Cells | mmu-miR-291a-3p | MIMAT0000368 | 19628328 |
| Germ Cells | mmu-miR-292-3p | MIMAT0000370 | 19628328 |
| Germ Cells | mmu-miR-293-3p | MIMAT0000371 | 19628328 |
| Germ Cells | mmu-miR-294-3p | MIMAT0000372 | 19628328 |
| Germ Cells | mmu-miR-295-3p | MIMAT0000373 | 19628328 |
| Germ Cells | mmu-miR-29b-3p | MIMAT0000127 | 19509302 |
| Germ Layers | hsa-miR-135b-5p | MIMAT0000758 | 19795981 |
| Germ Layers | hsa-miR-302a-3p | MIMAT0000684 | 21266536 |
| Germ Layers | hsa-miR-302a-3p | MIMAT0000684 | 19386261 |
| Germ Layers | hsa-miR-302d-3p | MIMAT0000718 | 21266536 |
| Germinal Center | hsa-miR-127-3p | MIMAT0000446 | 19530237 |
| Germinal Center | hsa-miR-155-5p | MIMAT0000646 | 19177201 |
| Germinal Center | mmu-miR-155-5p | MIMAT0000165 | 18055230 |
| Gonads | mmu-miR-29b-3p | MIMAT0000127 | 19509302 |
| Granulocyte Precursor Cells | hsa-miR-223-3p | MIMAT0000280 | 18278031 |
| Granulocyte Precursor Cells | mmu-miR-223-3p | MIMAT0000665 | 18278031 |
| Granulocyte Precursor Cells | mmu-miR-27a-3p | MIMAT0000537 | 19298589 |
| Granulocyte Precursor Cells | mmu-miR-27b-3p | MIMAT0000126 | 19298589 |
| Granulocytes | hsa-miR-146a-5p | MIMAT0000449 | 20375304 |
| Granulocytes | hsa-miR-148a-3p | MIMAT0000243 | 19435428 |
| Granulocytes | hsa-miR-19b-3p | MIMAT0000074 | 19435428 |
| Granulocytes | hsa-miR-20a-5p | MIMAT0000075 | 19435428 |
| Granulocytes | hsa-miR-214-3p | MIMAT0000271 | 19435428 |
| Granulocytes | hsa-miR-223-3p | MIMAT0000280 | 19017354 |
| Granulocytes | hsa-miR-223-3p | MIMAT0000280 | 18278031 |
| Granulocytes | hsa-miR-520h | MIMAT0002867 | 19435428 |
| Granulocytes | hsa-miR-92a-3p | MIMAT0000092 | 19435428 |
| Granulocytes | mmu-miR-223-3p | MIMAT0000665 | 18278031 |
| Granulocytes | mmu-miR-27a-3p | MIMAT0000537 | 19298589 |
| Granulocytes | mmu-miR-27b-3p | MIMAT0000126 | 19298589 |
| Granulosa Cells | hsa-miR-224-5p | MIMAT0000281 | 20118412 |
| Granulosa Cells | mmu-miR-503-5p | MIMAT0003188 | 19799966 |
| Granulosa Cells | rno-miR-125a-3p | MIMAT0004729 | 23045399 |
| Granulosa Cells | rno-miR-125a-5p | MIMAT0000829 | 23045399 |
| Granulosa Cells | rno-miR-455-5p | MIMAT0005316 | 23045399 |
| Growth Plate | mmu-miR-125b-5p | MIMAT0000136 | 21945074 |
| Growth Plate | mmu-miR-30a-5p | MIMAT0000128 | 21945074 |
| Growth Plate | mmu-miR-30c-5p | MIMAT0000514 | 21945074 |
| Hair | hsa-miR-199a-3p | MIMAT0000232 | 21807947 |
| Hair | hsa-miR-96-5p | MIMAT0000095 | 19363479 |
| Hair | mmu-miR-15a-5p | MIMAT0000526 | 19416898 |
| Hair | mmu-miR-31-5p | MIMAT0000538 | 20522784 |
| Hair | mmu-miR-96-5p | MIMAT0000541 | 19363478 |
| Hair Follicle | hsa-miR-199a-3p | MIMAT0000232 | 21807947 |
| Hair Follicle | mmu-miR-31-5p | MIMAT0000538 | 20522784 |
| Hand | hsa-let-7a-5p | MIMAT0000062 | 18679415 |
| Hand | hsa-let-7a-5p | MIMAT0000062 | 18758960 |
| Hand | hsa-miR-10a-5p | MIMAT0000253 | 20506192 |
| Hand | hsa-miR-125b-5p | MIMAT0000423 | 19570947 |
| Hand | hsa-miR-155-5p | MIMAT0000646 | 19538740 |
| Hand | hsa-miR-15a-5p | MIMAT0000068 | 17707831 |
| Hand | hsa-miR-16-5p | MIMAT0000069 | 17707831 |
| Hand | hsa-miR-17-5p | MIMAT0000070 | 18941111 |
| Hand | hsa-miR-18a-3p | MIMAT0002891 | 19372139 |
| Hand | hsa-miR-20a-5p | MIMAT0000075 | 18941111 |
| Hand | hsa-miR-21-5p | MIMAT0000076 | 21317927 |
| Hand | hsa-miR-29b-3p | MIMAT0000100 | 17707831 |
| Hand | hsa-miR-448 | MIMAT0001532 | 20798686 |
| Hand | hsa-miR-503-5p | MIMAT0002874 | 19538740 |
| Hand | hsa-miR-519c-3p | MIMAT0002832 | 19825807 |
| Hand | hsa-miR-520h | MIMAT0002867 | 19825807 |
| Hand | mmu-miR-100-3p | MIMAT0017051 | 20439489 |
| Hand | mmu-miR-100-5p | MIMAT0000655 | 20439489 |
| Hand | mmu-miR-137-3p | MIMAT0000149 | 20439489 |
| Hand | mmu-miR-137-5p | MIMAT0016986 | 20439489 |
| Hand | mmu-miR-195a-5p | MIMAT0000225 | 21622680 |
| Hand | mmu-miR-696 | MIMAT0003483 | 20086200 |
| Head | hsa-miR-101-3p | MIMAT0000099 | 19043531 |
| Head | hsa-miR-125a-5p | MIMAT0000443 | 19179615 |
| Head | hsa-miR-135b-5p | MIMAT0000758 | 23340180 |
| Head | hsa-miR-138-5p | MIMAT0000430 | 21770894 |
| Head | hsa-miR-15a-5p | MIMAT0000068 | 19117988 |
| Head | hsa-miR-181a-5p | MIMAT0000256 | 21274007 |
| Head | hsa-miR-188-5p | MIMAT0000457 | 19223510 |
| Head | hsa-miR-200c-3p | MIMAT0000617 | 21207483 |
| Head | hsa-miR-200c-3p | MIMAT0000617 | 19223510 |
| Head | hsa-miR-200c-3p | MIMAT0000617 | 21294122 |
| Head | hsa-miR-203a | MIMAT0000264 | 18483491 |
| Head | hsa-miR-204-5p | MIMAT0000265 | 20369013 |
| Head | hsa-miR-21-5p | MIMAT0000076 | 18372920 |
| Head | hsa-miR-30c-5p | MIMAT0000244 | 19223510 |
| Head | hsa-miR-30e-5p | MIMAT0000692 | 19223510 |
| Head | hsa-miR-31-3p | MIMAT0004504 | 20145132 |
| Head | hsa-miR-31-5p | MIMAT0000089 | 20145132 |
| Head | hsa-miR-34a-5p | MIMAT0000255 | 21294122 |
| Head | hsa-miR-34a-5p | MIMAT0000255 | 21240262 |
| Head | hsa-miR-374a-5p | MIMAT0000727 | 21274007 |
| Head | hsa-miR-489-3p | MIMAT0002805 | 20700123 |
| Head | hsa-miR-519a-3p | MIMAT0002869 | 21274007 |
| Head | hsa-miR-630 | MIMAT0003299 | 21274007 |
| Head | hsa-miR-98-5p | MIMAT0000096 | 17222355 |
| Head | mmu-miR-127-5p | MIMAT0004530 | 19878148 |
| Head | mmu-miR-204-5p | MIMAT0000237 | 20369013 |
| Head | mmu-miR-21a-5p | MIMAT0000530 | 18372920 |
| Head | mmu-miR-22-3p | MIMAT0000531 | 19878148 |
| Head | mmu-miR-27a-3p | MIMAT0000537 | 19878148 |
| Head | mmu-miR-29b-1-5p | MIMAT0004523 | 19878148 |
| Head | mmu-miR-382-3p | MIMAT0004691 | 19878148 |
| Head | mmu-miR-411-3p | MIMAT0001093 | 19878148 |
| Head | mmu-miR-470-5p | MIMAT0002111 | 19878148 |
| Head | mmu-miR-501-3p | MIMAT0003509 | 19878148 |
| Head | mmu-miR-669b-5p | MIMAT0003476 | 19878148 |
| Head | mmu-miR-676-3p | MIMAT0003782 | 19878148 |
| Head | mmu-miR-96-5p | MIMAT0000541 | 19878148 |
| Heart | hsa-miR-1 | MIMAT0000416 | 19131648 |
| Heart | hsa-miR-1 | MIMAT0000416 | 17401374 |
| Heart | hsa-miR-1 | MIMAT0000416 | 19188439 |
| Heart | hsa-miR-1 | MIMAT0000416 | 20571053 |
| Heart | hsa-miR-1 | MIMAT0000416 | 20081117 |
| Heart | hsa-miR-1 | MIMAT0000416 | 15951802 |
| Heart | hsa-miR-1 | MIMAT0000416 | 17443681 |
| Heart | hsa-miR-1 | MIMAT0000416 | 23141496 |
| Heart | hsa-miR-1 | MIMAT0000416 | 21169019 |
| Heart | hsa-miR-126-3p | MIMAT0000445 | 20571053 |
| Heart | hsa-miR-133a-3p | MIMAT0000427 | 19720047 |
| Heart | hsa-miR-133a-3p | MIMAT0000427 | 17443681 |
| Heart | hsa-miR-133a-3p | MIMAT0000427 | 23069713 |
| Heart | hsa-miR-133b | MIMAT0000770 | 19720047 |
| Heart | hsa-miR-150-5p | MIMAT0000451 | 23211718 |
| Heart | hsa-miR-155-5p | MIMAT0000646 | 19897480 |
| Heart | hsa-miR-182-5p | MIMAT0000259 | 17597072 |
| Heart | hsa-miR-199b-5p | MIMAT0000263 | 21102440 |
| Heart | hsa-miR-208a-3p | MIMAT0000241 | 17379774 |
| Heart | hsa-miR-21-5p | MIMAT0000076 | 23313253 |
| Heart | hsa-miR-21-5p | MIMAT0000076 | 20219857 |
| Heart | hsa-miR-22-3p | MIMAT0000077 | 22538858 |
| Heart | hsa-miR-223-3p | MIMAT0000280 | 20080987 |
| Heart | hsa-miR-27a-3p | MIMAT0000084 | 21149577 |
| Heart | hsa-miR-29a-3p | MIMAT0000086 | 21169019 |
| Heart | hsa-miR-320a | MIMAT0000510 | 19380620 |
| Heart | hsa-miR-34a-5p | MIMAT0000255 | 20627091 |
| Heart | hsa-miR-499a-5p | MIMAT0002870 | 20081117 |
| Heart | hsa-miR-499b-5p | MIMAT0019897 | 20081117 |
| Heart | hsa-miR-802 | MIMAT0004185 | 19897480 |
| Heart | hsa-miR-9-5p | MIMAT0000441 | 19188439 |
| Heart | hsa-miR-96-5p | MIMAT0000095 | 17597072 |
| Heart | mmu-miR-122-5p | MIMAT0000246 | 16258535 |
| Heart | mmu-miR-133a-3p | MIMAT0000145 | 17468766 |
| Heart | mmu-miR-133a-3p | MIMAT0000145 | 19043405 |
| Heart | mmu-miR-138-5p | MIMAT0000150 | 19004786 |
| Heart | mmu-miR-15b-5p | MIMAT0000124 | 20007690 |
| Heart | mmu-miR-16-5p | MIMAT0000527 | 20007690 |
| Heart | mmu-miR-195a-5p | MIMAT0000225 | 20007690 |
| Heart | mmu-miR-195a-5p | MIMAT0000225 | 21622680 |
| Heart | mmu-miR-199b-3p | MIMAT0004667 | 21102440 |
| Heart | mmu-miR-1a-3p | MIMAT0000123 | 17234972 |
| Heart | mmu-miR-1a-3p | MIMAT0000123 | 19188439 |
| Heart | mmu-miR-1a-3p | MIMAT0000123 | 19521018 |
| Heart | mmu-miR-1a-3p | MIMAT0000123 | 19933931 |
| Heart | mmu-miR-1a-3p | MIMAT0000123 | 15951802 |
| Heart | mmu-miR-1a-3p | MIMAT0000123 | 17397913 |
| Heart | mmu-miR-1a-3p | MIMAT0000123 | 23436819 |
| Heart | mmu-miR-206-3p | MIMAT0000239 | 19043405 |
| Heart | mmu-miR-208a-3p | MIMAT0000520 | 17379774 |
| Heart | mmu-miR-21a-5p | MIMAT0000530 | 19147652 |
| Heart | mmu-miR-21a-5p | MIMAT0000530 | 20404348 |
| Heart | mmu-miR-21a-5p | MIMAT0000530 | 19043405 |
| Heart | mmu-miR-27b-3p | MIMAT0000126 | 20736237 |
| Heart | mmu-miR-320-3p | MIMAT0000666 | 19380620 |
| Heart | mmu-miR-322-5p | MIMAT0000548 | 20007690 |
| Heart | mmu-miR-494-3p | MIMAT0003182 | 20837890 |
| Heart | rno-miR-1-3p | MIMAT0003125 | 17401374 |
| Heart | rno-miR-1-3p | MIMAT0003125 | 19188439 |
| Heart | rno-miR-1-3p | MIMAT0003125 | 17516552 |
| Heart | rno-miR-1-3p | MIMAT0003125 | 23436819 |
| Heart | rno-miR-133a-3p | MIMAT0000839 | 17516552 |
| Heart | rno-miR-133a-3p | MIMAT0000839 | 19720047 |
| Heart | rno-miR-133b-3p | MIMAT0003126 | 19720047 |
| Heart | rno-miR-15b-5p | MIMAT0000784 | 20007690 |
| Heart | rno-miR-16-5p | MIMAT0000785 | 20007690 |
| Heart | rno-miR-195-5p | MIMAT0000870 | 20007690 |
| Heart | rno-miR-21-5p | MIMAT0000790 | 19336275 |
| Heart | rno-miR-214-3p | MIMAT0000885 | 20007690 |
| Heart | rno-miR-23a-3p | MIMAT0000792 | 19574461 |
| Heart | rno-miR-27a-3p | MIMAT0000799 | 19574461 |
| Heart | rno-miR-27a-3p | MIMAT0000799 | 21149577 |
| Heart | rno-miR-29a-3p | MIMAT0000802 | 20164119 |
| Heart | rno-miR-29c-3p | MIMAT0000803 | 20164119 |
| Heart | rno-miR-322-5p | MIMAT0001619 | 20007690 |
| Heart | rno-miR-328a-3p | MIMAT0000564 | 21098446 |
| Heart Ventricles | hsa-miR-1 | MIMAT0000416 | 20571053 |
| Heart Ventricles | hsa-miR-126-3p | MIMAT0000445 | 20571053 |
| Heart Ventricles | rno-miR-21-5p | MIMAT0000790 | 19706597 |
| Hematopoietic Stem Cells | hsa-let-7d-5p | MIMAT0000065 | 20410187 |
| Hematopoietic Stem Cells | hsa-miR-125a-5p | MIMAT0000443 | 20616003 |
| Hematopoietic Stem Cells | hsa-miR-126-3p | MIMAT0000445 | 18474618 |
| Hematopoietic Stem Cells | hsa-miR-129-5p | MIMAT0000242 | 18189265 |
| Hematopoietic Stem Cells | hsa-miR-145-5p | MIMAT0000437 | 18474618 |
| Hematopoietic Stem Cells | hsa-miR-148a-3p | MIMAT0000243 | 19435428 |
| Hematopoietic Stem Cells | hsa-miR-155-5p | MIMAT0000646 | 18299402 |
| Hematopoietic Stem Cells | hsa-miR-19b-3p | MIMAT0000074 | 19435428 |
| Hematopoietic Stem Cells | hsa-miR-20a-5p | MIMAT0000075 | 19435428 |
| Hematopoietic Stem Cells | hsa-miR-214-3p | MIMAT0000271 | 19435428 |
| Hematopoietic Stem Cells | hsa-miR-223-3p | MIMAT0000280 | 19278969 |
| Hematopoietic Stem Cells | hsa-miR-24-3p | MIMAT0000080 | 17906079 |
| Hematopoietic Stem Cells | hsa-miR-520h | MIMAT0002867 | 18189265 |
| Hematopoietic Stem Cells | hsa-miR-520h | MIMAT0002867 | 19435428 |
| Hematopoietic Stem Cells | hsa-miR-92a-3p | MIMAT0000092 | 19435428 |
| Hematopoietic Stem Cells | mmu-let-7a-5p | MIMAT0000521 | 18474618 |
| Hematopoietic Stem Cells | mmu-miR-125b-5p | MIMAT0000136 | 21118986 |
| Hematopoietic Stem Cells | mmu-miR-126a-3p | MIMAT0000138 | 18474618 |
| Hematopoietic Stem Cells | mmu-miR-145a-5p | MIMAT0000157 | 18474618 |
| Hematopoietic System | hsa-miR-124-3p | MIMAT0000422 | 18451139 |
| Hematopoietic System | hsa-miR-155-5p | MIMAT0000646 | 19359473 |
| Hepatic Stellate Cells | hsa-miR-143-3p | MIMAT0000435 | 19913496 |
| Hepatic Stellate Cells | hsa-miR-218-5p | MIMAT0000275 | 19913496 |
| Hepatic Stellate Cells | hsa-miR-29b-3p | MIMAT0000100 | 19913496 |
| Hepatic Stellate Cells | mmu-miR-132-3p | MIMAT0000144 | 19843474 |
| Hepatic Stellate Cells | rno-miR-132-3p | MIMAT0000838 | 19843474 |
| Hepatic Stellate Cells | rno-miR-146a-5p | MIMAT0000852 | 22735812 |
| Hepatic Stellate Cells | rno-miR-150-5p | MIMAT0000853 | 19892940 |
| Hepatic Stellate Cells | rno-miR-15b-5p | MIMAT0000784 | 19232449 |
| Hepatic Stellate Cells | rno-miR-16-5p | MIMAT0000785 | 19232449 |
| Hepatic Stellate Cells | rno-miR-194-5p | MIMAT0000869 | 19892940 |
| Hepatic Stellate Cells | rno-miR-200a-3p | MIMAT0000874 | 19185571 |
| Hepatic Stellate Cells | rno-miR-27a-3p | MIMAT0000799 | 19185571 |
| Hepatic Stellate Cells | rno-miR-27b-3p | MIMAT0000798 | 19185571 |
| Hepatocytes | hsa-let-7c-5p | MIMAT0000064 | 20347499 |
| Hepatocytes | hsa-let-7g-5p | MIMAT0000414 | 20347499 |
| Hepatocytes | hsa-miR-103a-3p | MIMAT0000101 | 22723340 |
| Hepatocytes | hsa-miR-107 | MIMAT0000104 | 22723340 |
| Hepatocytes | hsa-miR-122-5p | MIMAT0000421 | 20351063 |
| Hepatocytes | hsa-miR-122-5p | MIMAT0000421 | 23055569 |
| Hepatocytes | hsa-miR-122-5p | MIMAT0000421 | 23221562 |
| Hepatocytes | hsa-miR-122-5p | MIMAT0000421 | 19726678 |
| Hepatocytes | hsa-miR-122-5p | MIMAT0000421 | 20842632 |
| Hepatocytes | hsa-miR-181a-5p | MIMAT0000256 | 22476949 |
| Hepatocytes | hsa-miR-181b-5p | MIMAT0000257 | 20023698 |
| Hepatocytes | hsa-miR-185-5p | MIMAT0000455 | 23459944 |
| Hepatocytes | hsa-miR-193b-3p | MIMAT0002819 | 20103677 |
| Hepatocytes | hsa-miR-196a-5p | MIMAT0000226 | 20127796 |
| Hepatocytes | hsa-miR-21-5p | MIMAT0000076 | 20827319 |
| Hepatocytes | hsa-miR-21-5p | MIMAT0000076 | 19072831 |
| Hepatocytes | hsa-miR-21-5p | MIMAT0000076 | 17681183 |
| Hepatocytes | hsa-miR-223-3p | MIMAT0000280 | 23459944 |
| Hepatocytes | hsa-miR-296-5p | MIMAT0000690 | 21633093 |
| Hepatocytes | hsa-miR-30a-5p | MIMAT0000087 | 19185580 |
| Hepatocytes | hsa-miR-33a-5p | MIMAT0000091 | 23547260 |
| Hepatocytes | hsa-miR-422a | MIMAT0001339 | 20351063 |
| Hepatocytes | hsa-miR-96-5p | MIMAT0000095 | 23459944 |
| Hepatocytes | mmu-miR-181b-5p | MIMAT0000673 | 20023698 |
| Hepatocytes | mmu-miR-194-5p | MIMAT0000224 | 21887698 |
| Hepatocytes | mmu-miR-21a-5p | MIMAT0000530 | 20432256 |
| Hepatocytes | mmu-miR-23b-3p | MIMAT0000125 | 19582816 |
| Hepatocytes | mmu-miR-24-3p | MIMAT0000219 | 19582816 |
| Hepatocytes | mmu-miR-27b-3p | MIMAT0000126 | 19582816 |
| Hepatocytes | mmu-miR-30a-5p | MIMAT0000128 | 19185580 |
| Hepatocytes | mmu-miR-378a-3p | MIMAT0003151 | 20432256 |
| Hepatocytes | mmu-miR-378a-5p | MIMAT0000742 | 20432256 |
| Hippocampus | hsa-miR-141-3p | MIMAT0000432 | 20584986 |
| Hippocampus | hsa-miR-21-5p | MIMAT0000076 | 20584986 |
| Hippocampus | hsa-miR-210-3p | MIMAT0000267 | 18539147 |
| Hippocampus | hsa-miR-375 | MIMAT0000728 | 20584986 |
| Hippocampus | mmu-miR-135a-5p | MIMAT0000147 | 20619502 |
| Hippocampus | mmu-miR-141-3p | MIMAT0000153 | 20584986 |
| Hippocampus | mmu-miR-16-5p | MIMAT0000527 | 20619502 |
| Hippocampus | mmu-miR-20a-5p | MIMAT0000529 | 20619502 |
| Hippocampus | mmu-miR-210-3p | MIMAT0000658 | 18539147 |
| Hippocampus | mmu-miR-298-5p | MIMAT0000376 | 18986979 |
| Hippocampus | mmu-miR-328-3p | MIMAT0000565 | 18986979 |
| Hippocampus | mmu-miR-375-3p | MIMAT0000739 | 20584986 |
| Hippocampus | rno-miR-141-3p | MIMAT0000846 | 20584986 |
| Hippocampus | rno-miR-21-5p | MIMAT0000790 | 20584986 |
| Hippocampus | rno-miR-21-5p | MIMAT0000790 | 21154462 |
| Hypothalamus | mmu-miR-7b-5p | MIMAT0000678 | 17028171 |
| Iliac Artery | rno-miR-21-5p | MIMAT0000790 | 22565856 |
| Immune System | hsa-miR-106a-5p | MIMAT0000103 | 19307576 |
| Immune System | hsa-miR-146a-5p | MIMAT0000449 | 20375304 |
| Immune System | hsa-miR-148a-3p | MIMAT0000243 | 19435428 |
| Immune System | hsa-miR-155-3p | MIMAT0004658 | 20852130 |
| Immune System | hsa-miR-155-5p | MIMAT0000646 | 20219467 |
| Immune System | hsa-miR-155-5p | MIMAT0000646 | 18299402 |
| Immune System | hsa-miR-155-5p | MIMAT0000646 | 21097505 |
| Immune System | hsa-miR-155-5p | MIMAT0000646 | 20852130 |
| Immune System | hsa-miR-155-5p | MIMAT0000646 | 19596814 |
| Immune System | hsa-miR-155-5p | MIMAT0000646 | 21030878 |
| Immune System | hsa-miR-155-5p | MIMAT0000646 | 20209161 |
| Immune System | hsa-miR-19b-3p | MIMAT0000074 | 19435428 |
| Immune System | hsa-miR-20a-5p | MIMAT0000075 | 19435428 |
| Immune System | hsa-miR-214-3p | MIMAT0000271 | 19435428 |
| Immune System | hsa-miR-520h | MIMAT0002867 | 19435428 |
| Immune System | hsa-miR-92a-3p | MIMAT0000092 | 19435428 |
| Immune System | mmu-miR-124-3p | MIMAT0000134 | 21131957 |
| Immune System | mmu-miR-146a-5p | MIMAT0000158 | 18791161 |
| Immune System | mmu-miR-155-5p | MIMAT0000165 | 18055230 |
| Immune System | mmu-miR-155-5p | MIMAT0000165 | 21385848 |
| Immune System | mmu-miR-155-5p | MIMAT0000165 | 17463290 |
| Induced Pluripotent Stem | hsa-miR-302a-3p | MIMAT0000684 | 23185040 |
| Cells | | | |
| Induced Pluripotent Stem | hsa-miR-302a-3p | MIMAT0000684 | 21062975 |
| Cells | | | |
| Induced Pluripotent Stem | hsa-miR-302b-3p | MIMAT0000715 | 23185040 |
| Cells | | | |
| Induced Pluripotent Stem | hsa-miR-302b-3p | MIMAT0000715 | 21062975 |
| Cells | | | |
| Induced Pluripotent Stem | hsa-miR-302c-3p | MIMAT0000717 | 23185040 |
| Cells | | | |
| Induced Pluripotent Stem | hsa-miR-302c-3p | MIMAT0000717 | 21062975 |
| Cells | | | |
| Induced Pluripotent Stem | hsa-miR-302d-3p | MIMAT0000718 | 23185040 |
| Cells | | | |
| Induced Pluripotent Stem | hsa-miR-302d-3p | MIMAT0000718 | 21062975 |
| Cells | | | |
| Induced Pluripotent Stem | mmu-let-7c-5p | MIMAT0000523 | 20054295 |
| Cells | | | |
| Induced Pluripotent Stem | mmu-miR-106b-5p | MIMAT0000386 | 21285944 |
| Cells | | | |
| Induced Pluripotent Stem | mmu-miR-93-5p | MIMAT0000540 | 21285944 |
| Cells | | | |
| Insulin-Secreting Cells | hsa-miR-9-5p | MIMAT0000441 | 16831872 |
| Insulin-Secreting Cells | mmu-miR-375-3p | MIMAT0000739 | 20224724 |
| Insulin-Secreting Cells | mmu-miR-9-5p | MIMAT0000142 | 21288303 |
| Insulin-Secreting Cells | rno-miR-9a-5p | MIMAT0000781 | 16831872 |
| Intercellular Junctions | hsa-miR-1 | MIMAT0000416 | 23142026 |
| Intercellular Junctions | hsa-miR-203a | MIMAT0000264 | 22101077 |
| Intercellular Junctions | hsa-miR-483-3p | MIMAT0002173 | 22101077 |
| Intercellular Junctions | hsa-miR-595 | MIMAT0003263 | 22101077 |
| Intestinal Mucosa | hsa-miR-221-3p | MIMAT0000278 | 21236259 |
| Intestine Small | hsa-miR-34a-5p | MIMAT0000255 | 21240262 |
| Intestine Small | hsa-miR-92b-3p | MIMAT0003218 | 21030610 |
| Intestine Small | mmu-miR-194-5p | MIMAT0000224 | 21887698 |
| Intestines | hsa-miR-203a | MIMAT0000264 | 22101077 |
| Intestines | hsa-miR-483-3p | MIMAT0002173 | 22101077 |
| Intestines | hsa-miR-595 | MIMAT0003263 | 22101077 |
| Jejunum | hsa-miR-626 | MIMAT0003295 | 19892711 |
| Jejunum | hsa-miR-7-5p | MIMAT0000252 | 19892711 |
| Jejunum | mmu-miR-706 | MIMAT0003496 | 19892711 |
| Joints | hsa-miR-19a-3p | MIMAT0000073 | 22105995 |
| Joints | hsa-miR-19b-3p | MIMAT0000074 | 22105995 |
| Jurkat Cells | hsa-miR-143-3p | MIMAT0000435 | 19464056 |
| Jurkat Cells | hsa-miR-181c-5p | MIMAT0000258 | 21112091 |
| Jurkat Cells | hsa-miR-221-3p | MIMAT0000278 | 21236259 |
| Jurkat Cells | hsa-miR-27b-3p | MIMAT0000419 | 16982751 |
| Jurkat Cells | hsa-miR-513a-5p | MIMAT0002877 | 19155478 |
| Keratinocytes | hsa-miR-105-5p | MIMAT0000102 | 19509287 |
| Keratinocytes | hsa-miR-125b-5p | MIMAT0000423 | 21412257 |
| Keratinocytes | hsa-miR-181a-5p | MIMAT0000256 | 21167132 |
| Keratinocytes | hsa-miR-203a | MIMAT0000264 | 18483491 |
| Keratinocytes | hsa-miR-203a | MIMAT0000264 | 17622355 |
| Keratinocytes | hsa-miR-203a | MIMAT0000264 | 22917968 |
| Keratinocytes | hsa-miR-24-3p | MIMAT0000080 | 23553486 |
| Keratinocytes | hsa-miR-31-5p | MIMAT0000089 | 23553486 |
| Keratinocytes | hsa-miR-31-5p | MIMAT0000089 | 23233723 |
| Keratinocytes | mmu-miR-31-5p | MIMAT0000538 | 20522784 |
| Kidney | hsa-miR-141-3p | MIMAT0000432 | 18925646 |
| Kidney | hsa-miR-15a-5p | MIMAT0000068 | 18949056 |
| Kidney | hsa-miR-17-5p | MIMAT0000070 | 19821056 |
| Kidney | hsa-miR-200a-3p | MIMAT0000682 | 19910931 |
| Kidney | hsa-miR-200b-3p | MIMAT0000318 | 19910931 |
| Kidney | hsa-miR-21-5p | MIMAT0000076 | 23292313 |
| Kidney | hsa-miR-212-3p | MIMAT0000269 | 22880819 |
| Kidney | hsa-miR-224-5p | MIMAT0000281 | 20180642 |
| Kidney | hsa-miR-27a-3p | MIMAT0000084 | 19513126 |
| Kidney | hsa-miR-27b-3p | MIMAT0000419 | 19581388 |
| Kidney | hsa-miR-429 | MIMAT0001536 | 19910931 |
| Kidney | hsa-miR-450a-5p | MIMAT0001545 | 18230805 |
| Kidney | hsa-miR-562 | MIMAT0003226 | 19789318 |
| Kidney | mmu-miR-122-5p | MIMAT0000246 | 16258535 |
| Kidney | mmu-miR-192-5p | MIMAT0000517 | 17360662 |
| Kidney | mmu-miR-194-5p | MIMAT0000224 | 21887698 |
| Kidney | mmu-miR-215-5p | MIMAT0000904 | 17360662 |
| Kidney | mmu-miR-216a-5p | MIMAT0000662 | 20713358 |
| Kidney | mmu-miR-216a-5p | MIMAT0000662 | 19543271 |
| Kidney | mmu-miR-217-5p | MIMAT0000679 | 19543271 |
| Kidney | mmu-miR-298-5p | MIMAT0000376 | 19581388 |
| Kidney | mmu-miR-29c-3p | MIMAT0000536 | 21310958 |
| Kidney | mmu-miR-93-5p | MIMAT0000540 | 20501654 |
| Kidney | rno-let-7a-1-3p | MIMAT0017085 | 24885635 |
| Kidney | rno-let-7a-2-3p | MIMAT0017086 | 24885635 |
| Kidney | rno-let-7a-5p | MIMAT0000774 | 24885635 |
| Kidney | rno-let-7b-3p | MIMAT0004705 | 24885635 |
| Kidney | rno-let-7b-5p | MIMAT0000775 | 24885635 |
| Kidney | rno-let-7c-1-3p | MIMAT0017087 | 24885635 |
| Kidney | rno-let-7c-2-3p | MIMAT0017088 | 24885635 |
| Kidney | rno-let-7c-5p | MIMAT0000776 | 24885635 |
| Kidney | rno-let-7d-3p | MIMAT0000563 | 24885635 |
| Kidney | rno-let-7d-5p | MIMAT0000562 | 24885635 |
| Kidney | rno-let-7e-3p | MIMAT0004706 | 24885635 |
| Kidney | rno-let-7e-5p | MIMAT0000777 | 24885635 |
| Kidney | rno-let-7f-1-3p | MIMAT0017089 | 24885635 |
| Kidney | rno-let-7f-2-3p | MIMAT0017090 | 24885635 |
| Kidney | rno-let-7f-5p | MIMAT0000778 | 24885635 |
| Kidney | rno-let-7i-3p | MIMAT0004707 | 24885635 |
| Kidney | rno-let-7i-5p | MIMAT0000779 | 24885635 |
| Kidney | rno-miR-1-3p | MIMAT0003125 | 24885635 |
| Kidney | rno-miR-100-5p | MIMAT0000822 | 24885635 |
| Kidney | rno-miR-101a-3p | MIMAT0000823 | 24885635 |
| Kidney | rno-miR-101a-5p | MIMAT0004726 | 24885635 |
| Kidney | rno-miR-101b-3p | MIMAT0000615 | 24885635 |
| Kidney | rno-miR-101b-5p | MIMAT0017045 | 24885635 |
| Kidney | rno-miR-103-1-5p | MIMAT0017114 | 24885635 |
| Kidney | rno-miR-103-3p | MIMAT0000824 | 24885635 |
| Kidney | rno-miR-106b-3p | MIMAT0004727 | 24885635 |
| Kidney | rno-miR-106b-5p | MIMAT0000825 | 24885635 |
| Kidney | rno-miR-107-3p | MIMAT0000826 | 24885635 |
| Kidney | rno-miR-107-5p | MIMAT0017115 | 24885635 |
| Kidney | rno-miR-10a-3p | MIMAT0004709 | 24885635 |
| Kidney | rno-miR-10a-5p | MIMAT0000782 | 24885635 |
| Kidney | rno-miR-10b-3p | MIMAT0017092 | 24885635 |
| Kidney | rno-miR-10b-5p | MIMAT0000783 | 24885635 |
| Kidney | rno-miR-122-3p | MIMAT0017116 | 24885635 |
| Kidney | rno-miR-122-5p | MIMAT0000827 | 24885635 |
| Kidney | rno-miR-124-3p | MIMAT0000828 | 24885635 |
| Kidney | rno-miR-1249 | MIMAT0017892 | 24885635 |
| Kidney | rno-miR-125a-3p | MIMAT0004729 | 24885635 |
| Kidney | rno-miR-125a-5p | MIMAT0000829 | 24885635 |
| Kidney | rno-miR-125b-1-3p | MIMAT0004730 | 24885635 |
| Kidney | rno-miR-125b-2-3p | MIMAT0026467 | 24885635 |
| Kidney | rno-miR-125b-5p | MIMAT0000830 | 24885635 |
| Kidney | rno-miR-126a-3p | MIMAT0000832 | 24885635 |
| Kidney | rno-miR-126a-5p | MIMAT0000831 | 24885635 |
| Kidney | rno-miR-126a-5p | MIMAT0000831 | 19102782 |
| Kidney | rno-miR-126a-5p | MIMAT0000831 | 19102782 |
| Kidney | rno-miR-127-5p | MIMAT0017117 | 24885635 |
| Kidney | rno-miR-128-1-5p | MIMAT0017118 | 19102782 |
| Kidney | rno-miR-128-1-5p | MIMAT0017118 | 19102782 |
| Kidney | rno-miR-128-3p | MIMAT0000834 | 24885635 |
| Kidney | rno-miR-129-1-3p | MIMAT0017120 | 24885635 |
| Kidney | rno-miR-129-2-3p | MIMAT0000601 | 24885635 |
| Kidney | rno-miR-129-5p | MIMAT0000600 | 24885635 |
| Kidney | rno-miR-1306-3p | MIMAT0024852 | 24885635 |
| Kidney | rno-miR-1306-5p | MIMAT0024851 | 24885635 |
| Kidney | rno-miR-130a-3p | MIMAT0000836 | 24885635 |
| Kidney | rno-miR-130b-3p | MIMAT0000837 | 24885635 |
| Kidney | rno-miR-130b-5p | MIMAT0017122 | 24885635 |
| Kidney | rno-miR-132-3p | MIMAT0000838 | 24885635 |
| Kidney | rno-miR-132-5p | MIMAT0017123 | 23326503 |
| Kidney | rno-miR-132-5p | MIMAT0017123 | 24885635 |
| Kidney | rno-miR-133a-3p | MIMAT0000839 | 24885635 |
| Kidney | rno-miR-133b-3p | MIMAT0003126 | 24885635 |
| Kidney | rno-miR-134-3p | MIMAT0017125 | 24885635 |
| Kidney | rno-miR-134-5p | MIMAT0000840 | 24885635 |
| Kidney | rno-miR-135a-5p | MIMAT0000841 | 24885635 |
| Kidney | rno-miR-135b-3p | MIMAT0017043 | 24885635 |
| Kidney | rno-miR-135b-5p | MIMAT0000611 | 24885635 |
| Kidney | rno-miR-136-3p | MIMAT0004733 | 24885635 |
| Kidney | rno-miR-136-5p | MIMAT0000842 | 19102782 |
| Kidney | rno-miR-136-5p | MIMAT0000842 | 19102782 |
| Kidney | rno-miR-137-3p | MIMAT0000843 | 24885635 |
| Kidney | rno-miR-138-1-3p | MIMAT0004734 | 24885635 |
| Kidney | rno-miR-138-2-3p | MIMAT0017127 | 24885635 |
| Kidney | rno-miR-138-5p | MIMAT0000844 | 24885635 |
| Kidney | rno-miR-139-5p | MIMAT0000845 | 24885635 |
| Kidney | rno-miR-140-5p | MIMAT0000573 | 24885635 |
| Kidney | rno-miR-141-3p | MIMAT0000846 | 24885635 |
| Kidney | rno-miR-142-3p | MIMAT0000848 | 24885635 |
| Kidney | rno-miR-142-5p | MIMAT0000847 | 24885635 |
| Kidney | rno-miR-143-3p | MIMAT0000849 | 24885635 |
| Kidney | rno-miR-144-3p | MIMAT0000850 | 24885635 |
| Kidney | rno-miR-144-5p | MIMAT0017130 | 24885635 |
| Kidney | rno-miR-145-5p | MIMAT0000851 | 24885635 |
| Kidney | rno-miR-146a-5p | MIMAT0000852 | 24885635 |
| Kidney | rno-miR-146b-5p | MIMAT0005595 | 24885635 |
| Kidney | rno-miR-146b-5p | MIMAT0005595 | 23326503 |
| Kidney | rno-miR-147 | MIMAT0005297 | 19102782 |
| Kidney | rno-miR-147 | MIMAT0005297 | 24885635 |
| Kidney | rno-miR-147 | MIMAT0005297 | 19102782 |
| Kidney | rno-miR-148b-3p | MIMAT0000579 | 24885635 |
| Kidney | rno-miR-148b-5p | MIMAT0004645 | 24885635 |
| Kidney | rno-miR-150-3p | MIMAT0017133 | 24885635 |
| Kidney | rno-miR-150-5p | MIMAT0000853 | 24885635 |
| Kidney | rno-miR-151-3p | MIMAT0000614 | 24885635 |
| Kidney | rno-miR-151-5p | MIMAT0000613 | 24885635 |
| Kidney | rno-miR-152-3p | MIMAT0000854 | 24885635 |
| Kidney | rno-miR-152-5p | MIMAT0017134 | 24885635 |
| Kidney | rno-miR-153-3p | MIMAT0000855 | 24885635 |
| Kidney | rno-miR-154-3p | MIMAT0017136 | 24885635 |
| Kidney | rno-miR-154-5p | MIMAT0000856 | 24885635 |
| Kidney | rno-miR-15b-3p | MIMAT0017093 | 24885635 |
| Kidney | rno-miR-15b-5p | MIMAT0000784 | 24885635 |
| Kidney | rno-miR-16-3p | MIMAT0017094 | 24885635 |
| Kidney | rno-miR-16-5p | MIMAT0000785 | 24885635 |
| Kidney | rno-miR-17-1-3p | MIMAT0004710 | 24885635 |
| Kidney | rno-miR-17-2-3p | MIMAT0017295 | 24885635 |
| Kidney | rno-miR-17-5p | MIMAT0000786 | 24885635 |
| Kidney | rno-miR-181a-1-3p | MIMAT0000884 | 24885635 |
| Kidney | rno-miR-181a-2-3p | MIMAT0017138 | 24885635 |
| Kidney | rno-miR-181a-5p | MIMAT0000858 | 24885635 |
| Kidney | rno-miR-181b-2-3p | MIMAT0017140 | 24885635 |
| Kidney | rno-miR-181b-5p | MIMAT0000859 | 19102782 |
| Kidney | rno-miR-181b-5p | MIMAT0000859 | 19102782 |
| Kidney | rno-miR-181b-5p | MIMAT0000859 | 24885635 |
| Kidney | rno-miR-181d-5p | MIMAT0005299 | 24885635 |
| Kidney | rno-miR-182 | MIMAT0005300 | 24885635 |
| Kidney | rno-miR-183-5p | MIMAT0000860 | 24885635 |
| Kidney | rno-miR-1839-3p | MIMAT0024844 | 24885635 |
| Kidney | rno-miR-1839-5p | MIMAT0024843 | 24885635 |
| Kidney | rno-miR-1843-3p | MIMAT0024848 | 24885635 |
| Kidney | rno-miR-1843-5p | MIMAT0024847 | 24885635 |
| Kidney | rno-miR-185-5p | MIMAT0000862 | 24885635 |
| Kidney | rno-miR-186-3p | MIMAT0017143 | 24885635 |
| Kidney | rno-miR-186-5p | MIMAT0000863 | 24885635 |
| Kidney | rno-miR-187-3p | MIMAT0000864 | 24885635 |
| Kidney | rno-miR-188-5p | MIMAT0005301 | 24885635 |
| Kidney | rno-miR-18a-5p | MIMAT0000787 | 24885635 |
| Kidney | rno-miR-190a-3p | MIMAT0017145 | 24885635 |
| Kidney | rno-miR-190a-5p | MIMAT0000865 | 24885635 |
| Kidney | rno-miR-190b-5p | MIMAT0005302 | 24885635 |
| Kidney | rno-miR-191a-5p | MIMAT0000866 | 24885635 |
| Kidney | rno-miR-192-3p | MIMAT0017147 | 24885635 |
| Kidney | rno-miR-192-5p | MIMAT0000867 | 20393144 |
| Kidney | rno-miR-192-5p | MIMAT0000867 | 24885635 |
| Kidney | rno-miR-194-3p | MIMAT0017148 | 24885635 |
| Kidney | rno-miR-194-5p | MIMAT0000869 | 24885635 |
| Kidney | rno-miR-195-3p | MIMAT0017149 | 24885635 |
| Kidney | rno-miR-195-5p | MIMAT0000870 | 24885635 |
| Kidney | rno-miR-196a-3p | MIMAT0004737 | 24885635 |
| Kidney | rno-miR-196a-5p | MIMAT0000871 | 19102782 |
| Kidney | rno-miR-196a-5p | MIMAT0000871 | 19102782 |
| Kidney | rno-miR-196a-5p | MIMAT0000871 | 24885635 |
| Kidney | rno-miR-196b-5p | MIMAT0001082 | 24885635 |
| Kidney | rno-miR-196c-3p | MIMAT0017299 | 24885635 |
| Kidney | rno-miR-196c-5p | MIMAT0005303 | 24885635 |
| Kidney | rno-miR-199a-3p | MIMAT0004738 | 24885635 |
| Kidney | rno-miR-199a-5p | MIMAT0000872 | 24885635 |
| Kidney | rno-miR-199a-5p | MIMAT0000872 | 23326503 |
| Kidney | rno-miR-19a-3p | MIMAT0000789 | 24885635 |
| Kidney | rno-miR-19a-5p | MIMAT0017098 | 24885635 |
| Kidney | rno-miR-19b-1-5p | MIMAT0017096 | 24885635 |
| Kidney | rno-miR-19b-3p | MIMAT0000788 | 24885635 |
| Kidney | rno-miR-200a-3p | MIMAT0000874 | 24885635 |
| Kidney | rno-miR-200b-3p | MIMAT0000875 | 24885635 |
| Kidney | rno-miR-200b-5p | MIMAT0017152 | 24885635 |
| Kidney | rno-miR-200c-3p | MIMAT0000873 | 24885635 |
| Kidney | rno-miR-201-3p | MIMAT0017364 | 24885635 |
| Kidney | rno-miR-203a-3p | MIMAT0000876 | 24885635 |
| Kidney | rno-miR-203a-5p | MIMAT0017153 | 19102782 |
| Kidney | rno-miR-203a-5p | MIMAT0017153 | 19102782 |
| Kidney | rno-miR-203a-5p | MIMAT0017153 | 24885635 |
| Kidney | rno-miR-203b-3p | MIMAT0017800 | 24885635 |
| Kidney | rno-miR-203b-5p | MIMAT0017799 | 24885635 |
| Kidney | rno-miR-204-3p | MIMAT0004739 | 24885635 |
| Kidney | rno-miR-204-5p | MIMAT0000877 | 24885635 |
| Kidney | rno-miR-205 | MIMAT0000878 | 24885635 |
| Kidney | rno-miR-206-3p | MIMAT0000879 | 24885635 |
| Kidney | rno-miR-20a-3p | MIMAT0000603 | 24885635 |
| Kidney | rno-miR-20a-5p | MIMAT0000602 | 19102782 |
| Kidney | rno-miR-20a-5p | MIMAT0000602 | 19102782 |
| Kidney | rno-miR-20a-5p | MIMAT0000602 | 24885635 |
| Kidney | rno-miR-20b-5p | MIMAT0003211 | 24885635 |
| Kidney | rno-miR-21-3p | MIMAT0004711 | 24885635 |
| Kidney | rno-miR-21-5p | MIMAT0000790 | 24885635 |
| Kidney | rno-miR-21-5p | MIMAT0000790 | 23326503 |
| Kidney | rno-miR-21-5p | MIMAT0000790 | 19102782 |
| Kidney | rno-miR-21-5p | MIMAT0000790 | 19102782 |
| Kidney | rno-miR-210-3p | MIMAT0000881 | 24885635 |
| Kidney | rno-miR-211-5p | MIMAT0000882 | 24885635 |
| Kidney | rno-miR-212-3p | MIMAT0000883 | 24885635 |
| Kidney | rno-miR-212-5p | MIMAT0017158 | 24885635 |
| Kidney | rno-miR-214-3p | MIMAT0000885 | 24885635 |
| Kidney | rno-miR-214-5p | MIMAT0017159 | 23326503 |
| Kidney | rno-miR-215 | MIMAT0003118 | 20393144 |
| Kidney | rno-miR-215 | MIMAT0003118 | 24885635 |
| Kidney | rno-miR-216a-5p | MIMAT0000886 | 24885635 |
| Kidney | rno-miR-216a-5p | MIMAT0000886 | 19102782 |
| Kidney | rno-miR-216a-5p | MIMAT0000886 | 19102782 |
| Kidney | rno-miR-217-5p | MIMAT0000887 | 24885635 |
| Kidney | rno-miR-217-5p | MIMAT0000887 | 19102782 |
| Kidney | rno-miR-217-5p | MIMAT0000887 | 19102782 |
| Kidney | rno-miR-218a-5p | MIMAT0000888 | 24885635 |
| Kidney | rno-miR-218b | MIMAT0017838 | 24885635 |
| Kidney | rno-miR-22-3p | MIMAT0000791 | 24885635 |
| Kidney | rno-miR-22-5p | MIMAT0003152 | 24885635 |
| Kidney | rno-miR-221-3p | MIMAT0000890 | 24885635 |
| Kidney | rno-miR-221-5p | MIMAT0017163 | 24885635 |
| Kidney | rno-miR-222-3p | MIMAT0000891 | 24885635 |
| Kidney | rno-miR-222-5p | MIMAT0017164 | 24885635 |
| Kidney | rno-miR-223-3p | MIMAT0000892 | 24885635 |
| Kidney | rno-miR-223-5p | MIMAT0017165 | 24885635 |
| Kidney | rno-miR-23a-3p | MIMAT0000792 | 24885635 |
| Kidney | rno-miR-23a-5p | MIMAT0004712 | 24885635 |
| Kidney | rno-miR-23b-3p | MIMAT0000793 | 24885635 |
| Kidney | rno-miR-23b-5p | MIMAT0017099 | 24885635 |
| Kidney | rno-miR-24-1-5p | MIMAT0003153 | 24885635 |
| Kidney | rno-miR-24-2-5p | MIMAT0005441 | 24885635 |
| Kidney | rno-miR-24-3p | MIMAT0000794 | 24885635 |
| Kidney | rno-miR-25-3p | MIMAT0000795 | 24885635 |
| Kidney | rno-miR-25-3p | MIMAT0000795 | 21071935 |
| Kidney | rno-miR-26a-5p | MIMAT0000796 | 24885635 |
| Kidney | rno-miR-26b-3p | MIMAT0004714 | 24885635 |
| Kidney | rno-miR-26b-5p | MIMAT0000797 | 24885635 |
| Kidney | rno-miR-27a-3p | MIMAT0000799 | 24885635 |
| Kidney | rno-miR-27a-5p | MIMAT0004715 | 24885635 |
| Kidney | rno-miR-27b-3p | MIMAT0000798 | 24885635 |
| Kidney | rno-miR-27b-5p | MIMAT0017101 | 24885635 |
| Kidney | rno-miR-28-3p | MIMAT0004716 | 24885635 |
| Kidney | rno-miR-28-5p | MIMAT0000800 | 24885635 |
| Kidney | rno-miR-299a-3p | MIMAT0017167 | 24885635 |
| Kidney | rno-miR-299a-5p | MIMAT0000901 | 24885635 |
| Kidney | rno-miR-299b-5p | MIMAT0017833 | 24885635 |
| Kidney | rno-miR-29a-3p | MIMAT0000802 | 24885635 |
| Kidney | rno-miR-29a-5p | MIMAT0004718 | 24885635 |
| Kidney | rno-miR-29b-1-5p | MIMAT0005445 | 24885635 |
| Kidney | rno-miR-29b-2-5p | MIMAT0004717 | 24885635 |
| Kidney | rno-miR-29b-3p | MIMAT0000801 | 24885635 |
| Kidney | rno-miR-29c-3p | MIMAT0000803 | 24885635 |
| Kidney | rno-miR-29c-5p | MIMAT0003154 | 24885635 |
| Kidney | rno-miR-300-3p | MIMAT0000902 | 24885635 |
| Kidney | rno-miR-300-5p | MIMAT0004743 | 24885635 |
| Kidney | rno-miR-301a-3p | MIMAT0000552 | 24885635 |
| Kidney | rno-miR-301a-5p | MIMAT0017026 | 24885635 |
| Kidney | rno-miR-301b-3p | MIMAT0005304 | 24885635 |
| Kidney | rno-miR-3065-3p | MIMAT0017840 | 24885635 |
| Kidney | rno-miR-3065-5p | MIMAT0017839 | 24885635 |
| Kidney | rno-miR-3072 | MIMAT0025071 | 24885635 |
| Kidney | rno-miR-3075 | MIMAT0025057 | 24885635 |
| Kidney | rno-miR-30a-3p | MIMAT0000809 | 24885635 |
| Kidney | rno-miR-30a-5p | MIMAT0000808 | 24885635 |
| Kidney | rno-miR-30a-5p | MIMAT0000808 | 19102782 |
| Kidney | rno-miR-30a-5p | MIMAT0000808 | 19102782 |
| Kidney | rno-miR-30b-3p | MIMAT0004721 | 24885635 |
| Kidney | rno-miR-30b-5p | MIMAT0000806 | 24885635 |
| Kidney | rno-miR-30c-1-3p | MIMAT0004719 | 24885635 |
| Kidney | rno-miR-30c-2-3p | MIMAT0005442 | 24885635 |
| Kidney | rno-miR-30c-5p | MIMAT0000804 | 24885635 |
| Kidney | rno-miR-30d-3p | MIMAT0004722 | 24885635 |
| Kidney | rno-miR-30d-5p | MIMAT0000807 | 24885635 |
| Kidney | rno-miR-30e-3p | MIMAT0004720 | 24885635 |
| Kidney | rno-miR-30e-5p | MIMAT0000805 | 24885635 |
| Kidney | rno-miR-31a-3p | MIMAT0017102 | 24885635 |
| Kidney | rno-miR-31a-5p | MIMAT0000810 | 24885635 |
| Kidney | rno-miR-31a-5p | MIMAT0000810 | 19102782 |
| Kidney | rno-miR-31a-5p | MIMAT0000810 | 19102782 |
| Kidney | rno-miR-31a-5p | MIMAT0000810 | 23326503 |
| Kidney | rno-miR-32-5p | MIMAT0000811 | 24885635 |
| Kidney | rno-miR-320-3p | MIMAT0000903 | 24885635 |
| Kidney | rno-miR-322-3p | MIMAT0000547 | 24885635 |
| Kidney | rno-miR-322-5p | MIMAT0001619 | 24885635 |
| Kidney | rno-miR-323-3p | MIMAT0000550 | 24885635 |
| Kidney | rno-miR-324-3p | MIMAT0000554 | 24885635 |
| Kidney | rno-miR-324-5p | MIMAT0000553 | 24885635 |
| Kidney | rno-miR-326-3p | MIMAT0000560 | 24885635 |
| Kidney | rno-miR-328a-3p | MIMAT0000564 | 24885635 |
| Kidney | rno-miR-328b-3p | MIMAT0017904 | 24885635 |
| Kidney | rno-miR-329-3p | MIMAT0000566 | 24885635 |
| Kidney | rno-miR-329-5p | MIMAT0017031 | 24885635 |
| Kidney | rno-miR-33-3p | MIMAT0017104 | 24885635 |
| Kidney | rno-miR-33-5p | MIMAT0000812 | 24885635 |
| Kidney | rno-miR-330-3p | MIMAT0000568 | 24885635 |
| Kidney | rno-miR-330-5p | MIMAT0004641 | 24885635 |
| Kidney | rno-miR-331-3p | MIMAT0000570 | 24885635 |
| Kidney | rno-miR-335 | MIMAT0000575 | 24885635 |
| Kidney | rno-miR-335 | MIMAT0000575 | 19102782 |
| Kidney | rno-miR-335 | MIMAT0000575 | 19102782 |
| Kidney | rno-miR-336-5p | MIMAT0000576 | 24885635 |
| Kidney | rno-miR-338-3p | MIMAT0000581 | 24885635 |
| Kidney | rno-miR-339-3p | MIMAT0004648 | 24885635 |
| Kidney | rno-miR-339-5p | MIMAT0000583 | 24885635 |
| Kidney | rno-miR-340-3p | MIMAT0000585 | 24885635 |
| Kidney | rno-miR-340-5p | MIMAT0004650 | 24885635 |
| Kidney | rno-miR-341 | MIMAT0000587 | 24885635 |
| Kidney | rno-miR-342-3p | MIMAT0000589 | 24885635 |
| Kidney | rno-miR-342-5p | MIMAT0004652 | 24885635 |
| Kidney | rno-miR-345-3p | MIMAT0004655 | 24885635 |
| Kidney | rno-miR-345-5p | MIMAT0000594 | 24885635 |
| Kidney | rno-miR-346 | MIMAT0000596 | 19102782 |
| Kidney | rno-miR-346 | MIMAT0000596 | 19102782 |
| Kidney | rno-miR-3473 | MIMAT0024853 | 24885635 |
| Kidney | rno-miR-34a-3p | MIMAT0017106 | 24885635 |
| Kidney | rno-miR-34a-5p | MIMAT0000815 | 23326503 |
| Kidney | rno-miR-34a-5p | MIMAT0000815 | 24885635 |
| Kidney | rno-miR-34b-5p | MIMAT0000813 | 19102782 |
| Kidney | rno-miR-34b-5p | MIMAT0000813 | 19102782 |
| Kidney | rno-miR-350 | MIMAT0000604 | 24885635 |
| Kidney | rno-miR-351-3p | MIMAT0017041 | 24885635 |
| Kidney | rno-miR-351-5p | MIMAT0000608 | 24885635 |
| Kidney | rno-miR-352 | MIMAT0000610 | 24885635 |
| Kidney | rno-miR-3548 | MIMAT0017806 | 24885635 |
| Kidney | rno-miR-3553 | MIMAT0017814 | 24885635 |
| Kidney | rno-miR-3556a | MIMAT0017821 | 24885635 |
| Kidney | rno-miR-3556b | MIMAT0017818 | 24885635 |
| Kidney | rno-miR-3557-3p | MIMAT0017820 | 24885635 |
| Kidney | rno-miR-3557-5p | MIMAT0017819 | 24885635 |
| Kidney | rno-miR-3570 | MIMAT0017850 | 24885635 |
| Kidney | rno-miR-3571 | MIMAT0017851 | 24885635 |
| Kidney | rno-miR-3574 | MIMAT0017860 | 24885635 |
| Kidney | rno-miR-3583-5p | MIMAT0017873 | 24885635 |
| Kidney | rno-miR-3585-5p | MIMAT0017878 | 24885635 |
| Kidney | rno-miR-3586-3p | MIMAT0017881 | 24885635 |
| Kidney | rno-miR-3587 | MIMAT0017883 | 24885635 |
| Kidney | rno-miR-3588 | MIMAT0017887 | 24885635 |
| Kidney | rno-miR-3589 | MIMAT0017888 | 24885635 |
| Kidney | rno-miR-3590-3p | MIMAT0017890 | 24885635 |
| Kidney | rno-miR-3590-5p | MIMAT0017889 | 24885635 |
| Kidney | rno-miR-3591 | MIMAT0017893 | 24885635 |
| Kidney | rno-miR-3596a | MIMAT0017886 | 24885635 |
| Kidney | rno-miR-3596b | MIMAT0017871 | 24885635 |
| Kidney | rno-miR-3596c | MIMAT0017877 | 24885635 |
| Kidney | rno-miR-3596d | MIMAT0017823 | 24885635 |
| Kidney | rno-miR-361-5p | MIMAT0003117 | 24885635 |
| Kidney | rno-miR-362-3p | MIMAT0017357 | 24885635 |
| Kidney | rno-miR-362-5p | MIMAT0012828 | 24885635 |
| Kidney | rno-miR-369-3p | MIMAT0003207 | 24885635 |
| Kidney | rno-miR-374-3p | MIMAT0017223 | 24885635 |
| Kidney | rno-miR-374-5p | MIMAT0003208 | 24885635 |
| Kidney | rno-miR-375-3p | MIMAT0005307 | 24885635 |
| Kidney | rno-miR-376a-3p | MIMAT0003198 | 24885635 |
| Kidney | rno-miR-376b-3p | MIMAT0003196 | 24885635 |
| Kidney | rno-miR-376b-5p | MIMAT0003195 | 24885635 |
| Kidney | rno-miR-376c-3p | MIMAT0003194 | 24885635 |
| Kidney | rno-miR-376c-5p | MIMAT0017219 | 24885635 |
| Kidney | rno-miR-377-3p | MIMAT0003123 | 24885635 |
| Kidney | rno-miR-377-5p | MIMAT0017203 | 19102782 |
| Kidney | rno-miR-377-5p | MIMAT0017203 | 19102782 |
| Kidney | rno-miR-378a-3p | MIMAT0003379 | 24885635 |
| Kidney | rno-miR-378a-5p | MIMAT0003378 | 24885635 |
| Kidney | rno-miR-378b | MIMAT0024855 | 24885635 |
| Kidney | rno-miR-379-3p | MIMAT0004791 | 24885635 |
| Kidney | rno-miR-379-5p | MIMAT0003192 | 19102782 |
| Kidney | rno-miR-379-5p | MIMAT0003192 | 19102782 |
| Kidney | rno-miR-379-5p | MIMAT0003192 | 24885635 |
| Kidney | rno-miR-380-3p | MIMAT0017302 | 24885635 |
| Kidney | rno-miR-380-5p | MIMAT0005308 | 24885635 |
| Kidney | rno-miR-380-5p | MIMAT0005308 | 19102782 |
| Kidney | rno-miR-380-5p | MIMAT0005308 | 19102782 |
| Kidney | rno-miR-381-3p | MIMAT0003199 | 24885635 |
| Kidney | rno-miR-382-5p | MIMAT0003201 | 24885635 |
| Kidney | rno-miR-409a-5p | MIMAT0003204 | 24885635 |
| Kidney | rno-miR-410-3p | MIMAT0005311 | 24885635 |
| Kidney | rno-miR-411-3p | MIMAT0017304 | 24885635 |
| Kidney | rno-miR-411-5p | MIMAT0005312 | 24885635 |
| Kidney | rno-miR-412-5p | MIMAT0017204 | 24885635 |
| Kidney | rno-miR-423-3p | MIMAT0005313 | 24885635 |
| Kidney | rno-miR-423-5p | MIMAT0017305 | 24885635 |
| Kidney | rno-miR-425-3p | MIMAT0017306 | 24885635 |
| Kidney | rno-miR-425-5p | MIMAT0005314 | 24885635 |
| Kidney | rno-miR-429 | MIMAT0001538 | 24885635 |
| Kidney | rno-miR-433-3p | MIMAT0001628 | 24885635 |
| Kidney | rno-miR-434-3p | MIMAT0005315 | 24885635 |
| Kidney | rno-miR-434-5p | MIMAT0017307 | 24885635 |
| Kidney | rno-miR-448-5p | MIMAT0017177 | 19102782 |
| Kidney | rno-miR-448-5p | MIMAT0017177 | 19102782 |
| Kidney | rno-miR-449a-5p | MIMAT0001543 | 24885635 |
| Kidney | rno-miR-450a-3p | MIMAT0017183 | 24885635 |
| Kidney | rno-miR-450a-5p | MIMAT0001547 | 24885635 |
| Kidney | rno-miR-450a-5p | MIMAT0001547 | 18230805 |
| Kidney | rno-miR-451-5p | MIMAT0001633 | 24885635 |
| Kidney | rno-miR-455-3p | MIMAT0017308 | 24885635 |
| Kidney | rno-miR-455-5p | MIMAT0005316 | 24885635 |
| Kidney | rno-miR-466b-1-3p | MIMAT0017285 | 24885635 |
| Kidney | rno-miR-466b-2-3p | MIMAT0017286 | 24885635 |
| Kidney | rno-miR-484 | MIMAT0005319 | 24885635 |
| Kidney | rno-miR-485-3p | MIMAT0017222 | 24885635 |
| Kidney | rno-miR-485-5p | MIMAT0003203 | 24885635 |
| Kidney | rno-miR-487b-3p | MIMAT0003200 | 24885635 |
| Kidney | rno-miR-488-3p | MIMAT0005341 | 24885635 |
| Kidney | rno-miR-490-3p | MIMAT0012823 | 24885635 |
| Kidney | rno-miR-490-5p | MIMAT0017356 | 24885635 |
| Kidney | rno-miR-494-3p | MIMAT0003193 | 24885635 |
| Kidney | rno-miR-494-5p | MIMAT0017218 | 24885635 |
| Kidney | rno-miR-496-3p | MIMAT0012860 | 24885635 |
| Kidney | rno-miR-497-3p | MIMAT0017230 | 24885635 |
| Kidney | rno-miR-497-5p | MIMAT0003383 | 24885635 |
| Kidney | rno-miR-499-3p | MIMAT0017227 | 24885635 |
| Kidney | rno-miR-499-5p | MIMAT0003381 | 24885635 |
| Kidney | rno-miR-500-3p | MIMAT0005321 | 24885635 |
| Kidney | rno-miR-501-3p | MIMAT0017198 | 24885635 |
| Kidney | rno-miR-503-3p | MIMAT0017224 | 24885635 |
| Kidney | rno-miR-503-5p | MIMAT0003213 | 23326503 |
| Kidney | rno-miR-503-5p | MIMAT0003213 | 24885635 |
| Kidney | rno-miR-504 | MIMAT0012830 | 24885635 |
| Kidney | rno-miR-509-3p | MIMAT0024850 | 24885635 |
| Kidney | rno-miR-509-5p | MIMAT0024849 | 24885635 |
| Kidney | rno-miR-511-3p | MIMAT0017358 | 24885635 |
| Kidney | rno-miR-532-3p | MIMAT0005323 | 24885635 |
| Kidney | rno-miR-532-5p | MIMAT0005322 | 24885635 |
| Kidney | rno-miR-539-3p | MIMAT0017212 | 24885635 |
| Kidney | rno-miR-541-3p | MIMAT0017213 | 24885635 |
| Kidney | rno-miR-541-5p | MIMAT0003177 | 24885635 |
| Kidney | rno-miR-542-3p | MIMAT0003179 | 24885635 |
| Kidney | rno-miR-542-5p | MIMAT0003178 | 24885635 |
| Kidney | rno-miR-543-3p | MIMAT0003175 | 24885635 |
| Kidney | rno-miR-544-3p | MIMAT0012831 | 24885635 |
| Kidney | rno-miR-547-3p | MIMAT0012851 | 24885635 |
| Kidney | rno-miR-582-3p | MIMAT0017360 | 24885635 |
| Kidney | rno-miR-582-5p | MIMAT0012833 | 24885635 |
| Kidney | rno-miR-598-3p | MIMAT0005325 | 24885635 |
| Kidney | rno-miR-615 | MIMAT0012835 | 24885635 |
| Kidney | rno-miR-6215 | MIMAT0024854 | 24885635 |
| Kidney | rno-miR-6329 | MIMAT0025068 | 24885635 |
| Kidney | rno-miR-6333 | MIMAT0025074 | 24885635 |
| Kidney | rno-miR-652-3p | MIMAT0005342 | 24885635 |
| Kidney | rno-miR-653-5p | MIMAT0012838 | 24885635 |
| Kidney | rno-miR-668 | MIMAT0012839 | 24885635 |
| Kidney | rno-miR-672-5p | MIMAT0005327 | 24885635 |
| Kidney | rno-miR-674-3p | MIMAT0005330 | 24885635 |
| Kidney | rno-miR-674-5p | MIMAT0005329 | 24885635 |
| Kidney | rno-miR-708-3p | MIMAT0005332 | 24885635 |
| Kidney | rno-miR-708-5p | MIMAT0005331 | 24885635 |
| Kidney | rno-miR-7a-2-3p | MIMAT0017091 | 24885635 |
| Kidney | rno-miR-7a-5p | MIMAT0000606 | 24885635 |
| Kidney | rno-miR-7a-5p | MIMAT0000606 | 19102782 |
| Kidney | rno-miR-7a-5p | MIMAT0000606 | 19102782 |
| Kidney | rno-miR-7b | MIMAT0000780 | 24885635 |
| Kidney | rno-miR-7b | MIMAT0000780 | 19102782 |
| Kidney | rno-miR-7b | MIMAT0000780 | 19102782 |
| Kidney | rno-miR-802-3p | MIMAT0017362 | 24885635 |
| Kidney | rno-miR-802-5p | MIMAT0012840 | 24885635 |
| Kidney | rno-miR-872-3p | MIMAT0005283 | 24885635 |
| Kidney | rno-miR-872-5p | MIMAT0005282 | 24885635 |
| Kidney | rno-miR-874-3p | MIMAT0005284 | 24885635 |
| Kidney | rno-miR-874-5p | MIMAT0017290 | 24885635 |
| Kidney | rno-miR-877 | MIMAT0005285 | 24885635 |
| Kidney | rno-miR-92a-1-5p | MIMAT0017107 | 24885635 |
| Kidney | rno-miR-92a-3p | MIMAT0000816 | 24885635 |
| Kidney | rno-miR-92b-3p | MIMAT0005340 | 21071935 |
| Kidney | rno-miR-92b-3p | MIMAT0005340 | 24885635 |
| Kidney | rno-miR-93-5p | MIMAT0000817 | 24885635 |
| Kidney | rno-miR-96-5p | MIMAT0000818 | 19102782 |
| Kidney | rno-miR-96-5p | MIMAT0000818 | 19102782 |
| Kidney | rno-miR-96-5p | MIMAT0000818 | 24885635 |
| Kidney | rno-miR-98-5p | MIMAT0000819 | 24885635 |
| Kidney | rno-miR-99a-3p | MIMAT0004724 | 24885635 |
| Kidney | rno-miR-99a-5p | MIMAT0000820 | 24885635 |
| Kidney | rno-miR-99a-5p | MIMAT0000820 | 19102782 |
| Kidney | rno-miR-99a-5p | MIMAT0000820 | 19102782 |
| Kidney | rno-miR-99b-3p | MIMAT0004725 | 24885635 |
| Kidney | rno-miR-99b-5p | MIMAT0000821 | 24885635 |
| Kidney | rno-miR-9a-3p | MIMAT0004708 | 24885635 |
| Kidney | rno-miR-9a-5p | MIMAT0000781 | 24885635 |
| Kidney | rno-miR-9b-5p | MIMAT0017835 | 24885635 |
| Killer Cells Natural | hsa-miR-15a-5p | MIMAT0000068 | 22379033 |
| Killer Cells Natural | hsa-miR-15b-5p | MIMAT0000417 | 22379033 |
| Killer Cells Natural | hsa-miR-16-5p | MIMAT0000069 | 22379033 |
| Killer Cells Natural | hsa-miR-29a-3p | MIMAT0000086 | 19584290 |
| Killer Cells Natural | hsa-miR-30c-1-3p | MIMAT0004674 | 22320217 |
| Killer Cells Natural | hsa-miR-520b | MIMAT0002843 | 19109132 |
| Killer Cells Natural | mmu-miR-223-3p | MIMAT0000665 | 20935160 |
| Knee | hsa-miR-9-5p | MIMAT0000441 | 19008124 |
| Kupffer Cells | hsa-miR-155-5p | MIMAT0000646 | 19783678 |
| Kupffer Cells | hsa-miR-199a-5p | MIMAT0000231 | 19783678 |
| Langerhans Cells | hsa-miR-146a-5p | MIMAT0000449 | 20375304 |
| Leukocytes | hsa-miR-126-3p | MIMAT0000445 | 18227515 |
| Leukocytes | hsa-miR-146a-5p | MIMAT0000449 | 19333922 |
| Leukocytes | hsa-miR-155-5p | MIMAT0000646 | 21310411 |
| Leukocytes | hsa-miR-21-5p | MIMAT0000076 | 21069438 |
| Leukocytes | hsa-miR-31-5p | MIMAT0000089 | 23233723 |
| Leukocytes | hsa-miR-320a | MIMAT0000510 | 18523662 |
| Leukocytes | mmu-miR-223-3p | MIMAT0000665 | 22043014 |
| Limb Buds | mmu-miR-137-3p | MIMAT0000149 | 21256124 |
| Limb Buds | mmu-miR-140-5p | MIMAT0000151 | 21872590 |
| Limb Buds | mmu-miR-153-3p | MIMAT0000163 | 21256124 |
| Limb Buds | mmu-miR-363-3p | MIMAT0000708 | 21256124 |
| Liver | bta-miR-122 | MIMAT0003849 | 18073344 |
| Liver | bta-miR-29a | MIMAT0003518 | 17652184 |
| Liver | bta-miR-29c | MIMAT0003829 | 17652184 |
| Liver | cfa-miR-122 | MIMAT0006619 | 18073344 |
| Liver | cfa-miR-29a | MIMAT0006626 | 17652184 |
| Liver | cfa-miR-29c | MIMAT0006705 | 17652184 |
| Liver | dre-miR-122 | MIMAT0001818 | 18073344 |
| Liver | dre-miR-29a | MIMAT0001802 | 17652184 |
| Liver | hsa-let-7b-5p | MIMAT0000063 | 16971064 |
| Liver | hsa-let-7c-3p | MIMAT0026472 | 19841744 |
| Liver | hsa-let-7c-5p | MIMAT0000064 | 19841744 |
| Liver | hsa-miR-1 | MIMAT0000416 | 18593903 |
| Liver | hsa-miR-1 | MIMAT0000416 | 22963810 |
| Liver | hsa-miR-101-3p | MIMAT0000099 | 19133651 |
| Liver | hsa-miR-103a-3p | MIMAT0000101 | 22723340 |
| Liver | hsa-miR-107 | MIMAT0000104 | 22723340 |
| Liver | hsa-miR-107 | MIMAT0000104 | 21029372 |
| Liver | hsa-miR-10b-5p | MIMAT0000254 | 19780876 |
| Liver | hsa-miR-122-5p | MIMAT0000421 | 19296470 |
| Liver | hsa-miR-122-5p | MIMAT0000421 | 20351063 |
| Liver | hsa-miR-122-5p | MIMAT0000421 | 17179747 |
| Liver | hsa-miR-122-5p | MIMAT0000421 | 17616664 |
| Liver | hsa-miR-122-5p | MIMAT0000421 | 19617899 |
| Liver | hsa-miR-122-5p | MIMAT0000421 | 18073344 |
| Liver | hsa-miR-122-5p | MIMAT0000421 | 19726678 |
| Liver | hsa-miR-122-5p | MIMAT0000421 | 23348614 |
| Liver | hsa-miR-122-5p | MIMAT0000421 | 22276989 |
| Liver | hsa-miR-122-5p | MIMAT0000421 | 16459310 |
| Liver | hsa-miR-122-5p | MIMAT0000421 | 20842632 |
| Liver | hsa-miR-124-3p | MIMAT0000422 | 19843643 |
| Liver | hsa-miR-125a-5p | MIMAT0000443 | 21220473 |
| Liver | hsa-miR-125a-5p | MIMAT0000443 | 16331254 |
| Liver | hsa-miR-125b-5p | MIMAT0000423 | 20827722 |
| Liver | hsa-miR-125b-5p | MIMAT0000423 | 18649363 |
| Liver | hsa-miR-140-5p | MIMAT0000431 | 23401231 |
| Liver | hsa-miR-141-3p | MIMAT0000432 | 20005803 |
| Liver | hsa-miR-143-3p | MIMAT0000435 | 19913496 |
| Liver | hsa-miR-143-3p | MIMAT0000435 | 16966691 |
| Liver | hsa-miR-143-3p | MIMAT0000435 | 19472311 |
| Liver | hsa-miR-145-5p | MIMAT0000437 | 23499894 |
| Liver | hsa-miR-145-5p | MIMAT0000437 | 22431718 |
| Liver | hsa-miR-152-3p | MIMAT0000438 | 20578129 |
| Liver | hsa-miR-155-5p | MIMAT0000646 | 19783678 |
| Liver | hsa-miR-15a-5p | MIMAT0000068 | 18949056 |
| Liver | hsa-miR-181a-5p | MIMAT0000256 | 22476949 |
| Liver | hsa-miR-181a-5p | MIMAT0000256 | 19585654 |
| Liver | hsa-miR-181b-5p | MIMAT0000257 | 19585654 |
| Liver | hsa-miR-181b-5p | MIMAT0000257 | 20023698 |
| Liver | hsa-miR-181c-5p | MIMAT0000258 | 19585654 |
| Liver | hsa-miR-181d-5p | MIMAT0002821 | 19585654 |
| Liver | hsa-miR-182-5p | MIMAT0000259 | 19167416 |
| Liver | hsa-miR-182-5p | MIMAT0000259 | 19569050 |
| Liver | hsa-miR-183-5p | MIMAT0000261 | 20979053 |
| Liver | hsa-miR-183-5p | MIMAT0000261 | 20602797 |
| Liver | hsa-miR-185-5p | MIMAT0000455 | 23459944 |
| Liver | hsa-miR-186-5p | MIMAT0000456 | 20979053 |
| Liver | hsa-miR-18a-5p | MIMAT0000072 | 19167416 |
| Liver | hsa-miR-18a-5p | MIMAT0000072 | 16331254 |
| Liver | hsa-miR-191-5p | MIMAT0000440 | 20924108 |
| Liver | hsa-miR-193b-3p | MIMAT0002819 | 20655737 |
| Liver | hsa-miR-194-5p | MIMAT0000460 | 19569050 |
| Liver | hsa-miR-195-5p | MIMAT0000461 | 16331254 |
| Liver | hsa-miR-196a-5p | MIMAT0000226 | 20127796 |
| Liver | hsa-miR-199a-3p | MIMAT0000232 | 16331254 |
| Liver | hsa-miR-199a-3p | MIMAT0000232 | 0 |
| Liver | hsa-miR-199a-5p | MIMAT0000231 | 20799954 |
| Liver | hsa-miR-199a-5p | MIMAT0000231 | 16331254 |
| Liver | hsa-miR-199a-5p | MIMAT0000231 | 19783678 |
| Liver | hsa-miR-200a-3p | MIMAT0000682 | 19167416 |
| Liver | hsa-miR-200a-3p | MIMAT0000682 | 20005803 |
| Liver | hsa-miR-200a-3p | MIMAT0000682 | 16331254 |
| Liver | hsa-miR-200b-3p | MIMAT0000318 | 19167416 |
| Liver | hsa-miR-200b-3p | MIMAT0000318 | 20005803 |
| Liver | hsa-miR-200b-3p | MIMAT0000318 | 19569050 |
| Liver | hsa-miR-200c-3p | MIMAT0000617 | 19167416 |
| Liver | hsa-miR-200c-3p | MIMAT0000617 | 20005803 |
| Liver | hsa-miR-200c-3p | MIMAT0000617 | 19569050 |
| Liver | hsa-miR-203a | MIMAT0000264 | 19843643 |
| Liver | hsa-miR-21-5p | MIMAT0000076 | 20827319 |
| Liver | hsa-miR-21-5p | MIMAT0000076 | 16966691 |
| Liver | hsa-miR-21-5p | MIMAT0000076 | 19072831 |
| Liver | hsa-miR-21-5p | MIMAT0000076 | 19133651 |
| Liver | hsa-miR-21-5p | MIMAT0000076 | 19167416 |
| Liver | hsa-miR-21-5p | MIMAT0000076 | 20167875 |
| Liver | hsa-miR-212-3p | MIMAT0000269 | 18162065 |
| Liver | hsa-miR-216a-5p | MIMAT0000273 | 23471579 |
| Liver | hsa-miR-217 | MIMAT0000274 | 23471579 |
| Liver | hsa-miR-218-5p | MIMAT0000275 | 19913496 |
| Liver | hsa-miR-22-3p | MIMAT0000077 | 0 |
| Liver | hsa-miR-221-3p | MIMAT0000278 | 20018759 |
| Liver | hsa-miR-222-3p | MIMAT0000279 | 20018759 |
| Liver | hsa-miR-222-3p | MIMAT0000279 | 20103675 |
| Liver | hsa-miR-223-3p | MIMAT0000280 | 23459944 |
| Liver | hsa-miR-223-3p | MIMAT0000280 | 18555017 |
| Liver | hsa-miR-224-5p | MIMAT0000281 | 16331254 |
| Liver | hsa-miR-26a-5p | MIMAT0000082 | 19524505 |
| Liver | hsa-miR-26b-5p | MIMAT0000083 | 19569050 |
| Liver | hsa-miR-296-5p | MIMAT0000690 | 21633093 |
| Liver | hsa-miR-29a-3p | MIMAT0000086 | 20943204 |
| Liver | hsa-miR-29a-3p | MIMAT0000086 | 21175813 |
| Liver | hsa-miR-29b-3p | MIMAT0000100 | 19913496 |
| Liver | hsa-miR-30a-5p | MIMAT0000087 | 19185580 |
| Liver | hsa-miR-320a | MIMAT0000510 | 22134529 |
| Liver | hsa-miR-320a | MIMAT0000510 | 19070389 |
| Liver | hsa-miR-33a-5p | MIMAT0000091 | 20466885 |
| Liver | hsa-miR-34a-5p | MIMAT0000255 | 20185821 |
| Liver | hsa-miR-34a-5p | MIMAT0000255 | 21240262 |
| Liver | hsa-miR-34a-5p | MIMAT0000255 | 19167416 |
| Liver | hsa-miR-374a-5p | MIMAT0000727 | 19569050 |
| Liver | hsa-miR-375 | MIMAT0000728 | 20226166 |
| Liver | hsa-miR-422a | MIMAT0001339 | 20351063 |
| Liver | hsa-miR-429 | MIMAT0001536 | 19569050 |
| Liver | hsa-miR-429 | MIMAT0001536 | 20005803 |
| Liver | hsa-miR-483-3p | MIMAT0002173 | 20388800 |
| Liver | hsa-miR-499a-5p | MIMAT0002870 | 21934092 |
| Liver | hsa-miR-499b-5p | MIMAT0019897 | 21934092 |
| Liver | hsa-miR-513a-5p | MIMAT0002877 | 19155478 |
| Liver | hsa-miR-631 | MIMAT0003300 | 20881232 |
| Liver | hsa-miR-758-3p | MIMAT0003879 | 21885853 |
| Liver | hsa-miR-96-5p | MIMAT0000095 | 23459944 |
| Liver | hsa-miR-96-5p | MIMAT0000095 | 19167416 |
| Liver | mmu-let-7c-5p | MIMAT0000523 | 17438130 |
| Liver | mmu-miR-122-5p | MIMAT0000246 | 16459310 |
| Liver | mmu-miR-122-5p | MIMAT0000246 | 20582318 |
| Liver | mmu-miR-122-5p | MIMAT0000246 | 17179747 |
| Liver | mmu-miR-122-5p | MIMAT0000246 | 18158304 |
| Liver | mmu-miR-122-5p | MIMAT0000246 | 19487572 |
| Liver | mmu-miR-122-5p | MIMAT0000246 | 16258535 |
| Liver | mmu-miR-122-5p | MIMAT0000246 | 21364282 |
| Liver | mmu-miR-127-5p | MIMAT0004530 | 19878148 |
| Liver | mmu-miR-132-3p | MIMAT0000144 | 19843474 |
| Liver | mmu-miR-181b-5p | MIMAT0000673 | 20023698 |
| Liver | mmu-miR-194-5p | MIMAT0000224 | 21887698 |
| Liver | mmu-miR-200b-3p | MIMAT0000233 | 19787069 |
| Liver | mmu-miR-200c-3p | MIMAT0000657 | 19787069 |
| Liver | mmu-miR-21a-5p | MIMAT0000530 | 20167875 |
| Liver | mmu-miR-21a-5p | MIMAT0000530 | 20432256 |
| Liver | mmu-miR-22-3p | MIMAT0000531 | 19878148 |
| Liver | mmu-miR-221-3p | MIMAT0000669 | 20018759 |
| Liver | mmu-miR-23b-3p | MIMAT0000125 | 19582816 |
| Liver | mmu-miR-24-3p | MIMAT0000219 | 19582816 |
| Liver | mmu-miR-27a-3p | MIMAT0000537 | 19878148 |
| Liver | mmu-miR-27b-3p | MIMAT0000126 | 19582816 |
| Liver | mmu-miR-29a-3p | MIMAT0000535 | 20943204 |
| Liver | mmu-miR-29a-3p | MIMAT0000535 | 17652184 |
| Liver | mmu-miR-29a-5p | MIMAT0004631 | 20943204 |
| Liver | mmu-miR-29b-1-5p | MIMAT0004523 | 19878148 |
| Liver | mmu-miR-29b-3p | MIMAT0000127 | 17652184 |
| Liver | mmu-miR-29c-3p | MIMAT0000536 | 17652184 |
| Liver | mmu-miR-30a-5p | MIMAT0000128 | 19185580 |
| Liver | mmu-miR-33-5p | MIMAT0000667 | 20466885 |
| Liver | mmu-miR-34a-5p | MIMAT0000542 | 21216258 |
| Liver | mmu-miR-378a-3p | MIMAT0003151 | 20432256 |
| Liver | mmu-miR-378a-5p | MIMAT0000742 | 20432256 |
| Liver | mmu-miR-382-3p | MIMAT0004691 | 19878148 |
| Liver | mmu-miR-411-3p | MIMAT0001093 | 19878148 |
| Liver | mmu-miR-429-3p | MIMAT0001537 | 19167416 |
| Liver | mmu-miR-470-5p | MIMAT0002111 | 19878148 |
| Liver | mmu-miR-501-3p | MIMAT0003509 | 19878148 |
| Liver | mmu-miR-669b-5p | MIMAT0003476 | 19878148 |
| Liver | mmu-miR-676-3p | MIMAT0003782 | 19878148 |
| Liver | mmu-miR-93-5p | MIMAT0000540 | 21216258 |
| Liver | mmu-miR-96-5p | MIMAT0000541 | 19878148 |
| Liver | ppy-miR-122 | MIMAT0015746 | 18073344 |
| Liver | ppy-miR-29a | MIMAT0002363 | 17652184 |
| Liver | ppy-miR-29c | MIMAT0015735 | 17652184 |
| Liver | ptr-miR-122 | MIMAT0007963 | 18073344 |
| Liver | ptr-miR-29a | MIMAT0002364 | 17652184 |
| Liver | ptr-miR-29c | MIMAT0008082 | 17652184 |
| Liver | rno-let-7a-5p | MIMAT0000774 | 0 |
| Liver | rno-let-7b-5p | MIMAT0000775 | 0 |
| Liver | rno-let-7c-5p | MIMAT0000776 | 0 |
| Liver | rno-let-7d-3p | MIMAT0000563 | 0 |
| Liver | rno-let-7d-5p | MIMAT0000562 | 0 |
| Liver | rno-let-7e-5p | MIMAT0000777 | 0 |
| Liver | rno-let-7f-2-3p | MIMAT0017090 | 0 |
| Liver | rno-let-7f-5p | MIMAT0000778 | 0 |
| Liver | rno-let-7i-3p | MIMAT0004707 | 0 |
| Liver | rno-let-7i-5p | MIMAT0000779 | 0 |
| Liver | rno-miR-1-3p | MIMAT0003125 | 0 |
| Liver | rno-miR-100-5p | MIMAT0000822 | 0 |
| Liver | rno-miR-101a-3p | MIMAT0000823 | 0 |
| Liver | rno-miR-101b-3p | MIMAT0000615 | 0 |
| Liver | rno-miR-103-3p | MIMAT0000824 | 0 |
| Liver | rno-miR-107-3p | MIMAT0000826 | 0 |
| Liver | rno-miR-10a-5p | MIMAT0000782 | 0 |
| Liver | rno-miR-122-3p | MIMAT0017116 | 0 |
| Liver | rno-miR-122-5p | MIMAT0000827 | 0 |
| Liver | rno-miR-122-5p | MIMAT0000827 | 18073344 |
| Liver | rno-miR-125a-3p | MIMAT0004729 | 23045399 |
| Liver | rno-miR-125a-5p | MIMAT0000829 | 0 |
| Liver | rno-miR-125a-5p | MIMAT0000829 | 23045399 |
| Liver | rno-miR-125b-1-3p | MIMAT0004730 | 0 |
| Liver | rno-miR-125b-2-3p | MIMAT0026467 | 0 |
| Liver | rno-miR-125b-5p | MIMAT0000830 | 0 |
| Liver | rno-miR-126a-3p | MIMAT0000832 | 0 |
| Liver | rno-miR-126a-5p | MIMAT0000831 | 0 |
| Liver | rno-miR-127-3p | MIMAT0000833 | 18942116 |
| Liver | rno-miR-127-3p | MIMAT0000833 | 0 |
| Liver | rno-miR-128-3p | MIMAT0000834 | 0 |
| Liver | rno-miR-1306-5p | MIMAT0024851 | 0 |
| Liver | rno-miR-130a-3p | MIMAT0000836 | 0 |
| Liver | rno-miR-130b-3p | MIMAT0000837 | 0 |
| Liver | rno-miR-132-3p | MIMAT0000838 | 19843474 |
| Liver | rno-miR-133a-3p | MIMAT0000839 | 0 |
| Liver | rno-miR-133b-3p | MIMAT0003126 | 0 |
| Liver | rno-miR-135a-5p | MIMAT0000841 | 0 |
| Liver | rno-miR-135b-5p | MIMAT0000611 | 0 |
| Liver | rno-miR-136-3p | MIMAT0004733 | 0 |
| Liver | rno-miR-136-5p | MIMAT0000842 | 0 |
| Liver | rno-miR-137-3p | MIMAT0000843 | 0 |
| Liver | rno-miR-140-3p | MIMAT0000574 | 0 |
| Liver | rno-miR-140-5p | MIMAT0000573 | 0 |
| Liver | rno-miR-141-3p | MIMAT0000846 | 0 |
| Liver | rno-miR-142-3p | MIMAT0000848 | 0 |
| Liver | rno-miR-142-5p | MIMAT0000847 | 0 |
| Liver | rno-miR-143-5p | MIMAT0017129 | 0 |
| Liver | rno-miR-144-3p | MIMAT0000850 | 0 |
| Liver | rno-miR-146a-5p | MIMAT0000852 | 22735812 |
| Liver | rno-miR-146b-5p | MIMAT0005595 | 0 |
| Liver | rno-miR-148b-3p | MIMAT0000579 | 0 |
| Liver | rno-miR-150-5p | MIMAT0000853 | 19892940 |
| Liver | rno-miR-151-3p | MIMAT0000614 | 0 |
| Liver | rno-miR-152-3p | MIMAT0000854 | 0 |
| Liver | rno-miR-15b-5p | MIMAT0000784 | 0 |
| Liver | rno-miR-16-5p | MIMAT0000785 | 0 |
| Liver | rno-miR-17-1-3p | MIMAT0004710 | 0 |
| Liver | rno-miR-17-5p | MIMAT0000786 | 0 |
| Liver | rno-miR-182 | MIMAT0005300 | 19167416 |
| Liver | rno-miR-185-5p | MIMAT0000862 | 0 |
| Liver | rno-miR-186-3p | MIMAT0017143 | 0 |
| Liver | rno-miR-186-5p | MIMAT0000863 | 0 |
| Liver | rno-miR-188-5p | MIMAT0005301 | 0 |
| Liver | rno-miR-18a-5p | MIMAT0000787 | 19167416 |
| Liver | rno-miR-190a-3p | MIMAT0017145 | 0 |
| Liver | rno-miR-190a-5p | MIMAT0000865 | 0 |
| Liver | rno-miR-190b-5p | MIMAT0005302 | 0 |
| Liver | rno-miR-191a-5p | MIMAT0000866 | 0 |
| Liver | rno-miR-192-5p | MIMAT0000867 | 0 |
| Liver | rno-miR-194-5p | MIMAT0000869 | 19892940 |
| Liver | rno-miR-194-5p | MIMAT0000869 | 0 |
| Liver | rno-miR-1949 | MIMAT0017852 | 0 |
| Liver | rno-miR-195-5p | MIMAT0000870 | 0 |
| Liver | rno-miR-199a-3p | MIMAT0004738 | 0 |
| Liver | rno-miR-199a-5p | MIMAT0000872 | 0 |
| Liver | rno-miR-19a-3p | MIMAT0000789 | 0 |
| Liver | rno-miR-19b-3p | MIMAT0000788 | 0 |
| Liver | rno-miR-200a-3p | MIMAT0000874 | 19167416 |
| Liver | rno-miR-200a-3p | MIMAT0000874 | 19185571 |
| Liver | rno-miR-200b-3p | MIMAT0000875 | 19167416 |
| Liver | rno-miR-200b-3p | MIMAT0000875 | 18942116 |
| Liver | rno-miR-200c-3p | MIMAT0000873 | 19167416 |
| Liver | rno-miR-200c-3p | MIMAT0000873 | 0 |
| Liver | rno-miR-203a-3p | MIMAT0000876 | 0 |
| Liver | rno-miR-203a-5p | MIMAT0017153 | 0 |
| Liver | rno-miR-204-5p | MIMAT0000877 | 0 |
| Liver | rno-miR-206-3p | MIMAT0000879 | 0 |
| Liver | rno-miR-20a-3p | MIMAT0000603 | 0 |
| Liver | rno-miR-20a-5p | MIMAT0000602 | 0 |
| Liver | rno-miR-20b-5p | MIMAT0003211 | 0 |
| Liver | rno-miR-21-3p | MIMAT0004711 | 0 |
| Liver | rno-miR-21-5p | MIMAT0000790 | 0 |
| Liver | rno-miR-21-5p | MIMAT0000790 | 19167416 |
| Liver | rno-miR-210-3p | MIMAT0000881 | 0 |
| Liver | rno-miR-211-5p | MIMAT0000882 | 0 |
| Liver | rno-miR-215 | MIMAT0003118 | 0 |
| Liver | rno-miR-216a-5p | MIMAT0000886 | 0 |
| Liver | rno-miR-216b-5p | MIMAT0017846 | 0 |
| Liver | rno-miR-217-5p | MIMAT0000887 | 0 |
| Liver | rno-miR-218a-5p | MIMAT0000888 | 0 |
| Liver | rno-miR-22-3p | MIMAT0000791 | 0 |
| Liver | rno-miR-22-5p | MIMAT0003152 | 0 |
| Liver | rno-miR-221-3p | MIMAT0000890 | 0 |
| Liver | rno-miR-221-5p | MIMAT0017163 | 0 |
| Liver | rno-miR-223-3p | MIMAT0000892 | 0 |
| Liver | rno-miR-23a-3p | MIMAT0000792 | 0 |
| Liver | rno-miR-23b-3p | MIMAT0000793 | 0 |
| Liver | rno-miR-23b-5p | MIMAT0017099 | 0 |
| Liver | rno-miR-24-3p | MIMAT0000794 | 0 |
| Liver | rno-miR-25-3p | MIMAT0000795 | 0 |
| Liver | rno-miR-26a-5p | MIMAT0000796 | 0 |
| Liver | rno-miR-26b-5p | MIMAT0000797 | 0 |
| Liver | rno-miR-27a-3p | MIMAT0000799 | 0 |
| Liver | rno-miR-27a-3p | MIMAT0000799 | 19185571 |
| Liver | rno-miR-27b-3p | MIMAT0000798 | 0 |
| Liver | rno-miR-27b-3p | MIMAT0000798 | 19185571 |
| Liver | rno-miR-28-3p | MIMAT0004716 | 0 |
| Liver | rno-miR-29a-3p | MIMAT0000802 | 0 |
| Liver | rno-miR-29a-3p | MIMAT0000802 | 17652184 |
| Liver | rno-miR-29a-5p | MIMAT0004718 | 0 |
| Liver | rno-miR-29b-3p | MIMAT0000801 | 0 |
| Liver | rno-miR-29b-3p | MIMAT0000801 | 17652184 |
| Liver | rno-miR-29c-3p | MIMAT0000803 | 0 |
| Liver | rno-miR-29c-3p | MIMAT0000803 | 17652184 |
| Liver | rno-miR-30a-3p | MIMAT0000809 | 0 |
| Liver | rno-miR-30a-5p | MIMAT0000808 | 0 |
| Liver | rno-miR-30b-3p | MIMAT0004721 | 0 |
| Liver | rno-miR-30b-5p | MIMAT0000806 | 0 |
| Liver | rno-miR-30c-1-3p | MIMAT0004719 | 0 |
| Liver | rno-miR-30c-2-3p | MIMAT0005442 | 0 |
| Liver | rno-miR-30c-5p | MIMAT0000804 | 0 |
| Liver | rno-miR-30d-3p | MIMAT0004722 | 0 |
| Liver | rno-miR-30d-5p | MIMAT0000807 | 0 |
| Liver | rno-miR-30e-3p | MIMAT0004720 | 0 |
| Liver | rno-miR-30e-5p | MIMAT0000805 | 0 |
| Liver | rno-miR-31a-3p | MIMAT0017102 | 0 |
| Liver | rno-miR-31a-5p | MIMAT0000810 | 0 |
| Liver | rno-miR-320-3p | MIMAT0000903 | 0 |
| Liver | rno-miR-322-5p | MIMAT0001619 | 0 |
| Liver | rno-miR-329-5p | MIMAT0017031 | 0 |
| Liver | rno-miR-33-5p | MIMAT0000812 | 0 |
| Liver | rno-miR-335 | MIMAT0000575 | 0 |
| Liver | rno-miR-340-5p | MIMAT0004650 | 0 |
| Liver | rno-miR-341 | MIMAT0000587 | 0 |
| Liver | rno-miR-342-3p | MIMAT0000589 | 0 |
| Liver | rno-miR-3473 | MIMAT0024853 | 0 |
| Liver | rno-miR-34a-3p | MIMAT0017106 | 0 |
| Liver | rno-miR-34a-5p | MIMAT0000815 | 18942116 |
| Liver | rno-miR-34a-5p | MIMAT0000815 | 0 |
| Liver | rno-miR-34a-5p | MIMAT0000815 | 19167416 |
| Liver | rno-miR-350 | MIMAT0000604 | 0 |
| Liver | rno-miR-3556a | MIMAT0017821 | 0 |
| Liver | rno-miR-3556b | MIMAT0017818 | 0 |
| Liver | rno-miR-3557-3p | MIMAT0017820 | 0 |
| Liver | rno-miR-3557-5p | MIMAT0017819 | 0 |
| Liver | rno-miR-3558-5p | MIMAT0017825 | 0 |
| Liver | rno-miR-3559-3p | MIMAT0017828 | 0 |
| Liver | rno-miR-3571 | MIMAT0017851 | 0 |
| Liver | rno-miR-3574 | MIMAT0017860 | 0 |
| Liver | rno-miR-3586-3p | MIMAT0017881 | 0 |
| Liver | rno-miR-3587 | MIMAT0017883 | 0 |
| Liver | rno-miR-3588 | MIMAT0017887 | 0 |
| Liver | rno-miR-3590-3p | MIMAT0017890 | 0 |
| Liver | rno-miR-3591 | MIMAT0017893 | 0 |
| Liver | rno-miR-3596a | MIMAT0017886 | 0 |
| Liver | rno-miR-3596b | MIMAT0017871 | 0 |
| Liver | rno-miR-3596c | MIMAT0017877 | 0 |
| Liver | rno-miR-3596d | MIMAT0017823 | 0 |
| Liver | rno-miR-362-3p | MIMAT0017357 | 0 |
| Liver | rno-miR-362-5p | MIMAT0012828 | 0 |
| Liver | rno-miR-375-3p | MIMAT0005307 | 0 |
| Liver | rno-miR-376a-3p | MIMAT0003198 | 0 |
| Liver | rno-miR-378a-3p | MIMAT0003379 | 0 |
| Liver | rno-miR-378a-5p | MIMAT0003378 | 0 |
| Liver | rno-miR-378b | MIMAT0024855 | 0 |
| Liver | rno-miR-379-5p | MIMAT0003192 | 0 |
| Liver | rno-miR-423-5p | MIMAT0017305 | 0 |
| Liver | rno-miR-425-5p | MIMAT0005314 | 0 |
| Liver | rno-miR-429 | MIMAT0001538 | 0 |
| Liver | rno-miR-429 | MIMAT0001538 | 19167416 |
| Liver | rno-miR-449a-5p | MIMAT0001543 | 0 |
| Liver | rno-miR-450a-5p | MIMAT0001547 | 0 |
| Liver | rno-miR-451-5p | MIMAT0001633 | 0 |
| Liver | rno-miR-455-3p | MIMAT0017308 | 0 |
| Liver | rno-miR-455-5p | MIMAT0005316 | 0 |
| Liver | rno-miR-455-5p | MIMAT0005316 | 23045399 |
| Liver | rno-miR-466b-1-3p | MIMAT0017285 | 0 |
| Liver | rno-miR-466b-2-3p | MIMAT0017286 | 0 |
| Liver | rno-miR-466b-5p | MIMAT0005278 | 0 |
| Liver | rno-miR-466c-5p | MIMAT0005279 | 0 |
| Liver | rno-miR-497-5p | MIMAT0003383 | 0 |
| Liver | rno-miR-499-3p | MIMAT0017227 | 0 |
| Liver | rno-miR-499-5p | MIMAT0003381 | 0 |
| Liver | rno-miR-501-3p | MIMAT0017198 | 0 |
| Liver | rno-miR-501-5p | MIMAT0003116 | 0 |
| Liver | rno-miR-503-5p | MIMAT0003213 | 0 |
| Liver | rno-miR-511-3p | MIMAT0017358 | 0 |
| Liver | rno-miR-532-3p | MIMAT0005323 | 0 |
| Liver | rno-miR-532-5p | MIMAT0005322 | 0 |
| Liver | rno-miR-542-5p | MIMAT0003178 | 0 |
| Liver | rno-miR-547-3p | MIMAT0012851 | 0 |
| Liver | rno-miR-582-5p | MIMAT0012833 | 0 |
| Liver | rno-miR-6215 | MIMAT0024854 | 0 |
| Liver | rno-miR-664-1-5p | MIMAT0017228 | 0 |
| Liver | rno-miR-664-2-5p | MIMAT0017229 | 0 |
| Liver | rno-miR-664-3p | MIMAT0003382 | 0 |
| Liver | rno-miR-674-5p | MIMAT0005329 | 0 |
| Liver | rno-miR-708-5p | MIMAT0005331 | 0 |
| Liver | rno-miR-743b-3p | MIMAT0005280 | 0 |
| Liver | rno-miR-7a-1-3p | MIMAT0000607 | 0 |
| Liver | rno-miR-7a-5p | MIMAT0000606 | 0 |
| Liver | rno-miR-7b | MIMAT0000780 | 0 |
| Liver | rno-miR-802-3p | MIMAT0017362 | 0 |
| Liver | rno-miR-802-5p | MIMAT0012840 | 0 |
| Liver | rno-miR-872-3p | MIMAT0005283 | 0 |
| Liver | rno-miR-872-5p | MIMAT0005282 | 0 |
| Liver | rno-miR-92a-1-5p | MIMAT0017107 | 0 |
| Liver | rno-miR-92a-3p | MIMAT0000816 | 0 |
| Liver | rno-miR-92b-3p | MIMAT0005340 | 0 |
| Liver | rno-miR-96-5p | MIMAT0000818 | 0 |
| Liver | rno-miR-96-5p | MIMAT0000818 | 19167416 |
| Liver | rno-miR-98-5p | MIMAT0000819 | 0 |
| Liver | rno-miR-99a-5p | MIMAT0000820 | 0 |
| Liver | rno-miR-99b-5p | MIMAT0000821 | 0 |
| Liver | ssc-miR-122 | MIMAT0002119 | 18073344 |
| Liver | ssc-miR-29a | MIMAT0013870 | 17652184 |
| Liver | ssc-miR-29c | MIMAT0002166 | 17652184 |
| Liver | xtr-miR-122 | MIMAT0003585 | 18073344 |
| Liver | xtr-miR-29a | MIMAT0003656 | 17652184 |
| Locus Coeruleus | mmu-miR-16-5p | MIMAT0000527 | 20847275 |
| Lung | bta-miR-335 | MIMAT0009291 | 18185580 |
| Lung | cfa-miR-335 | MIMAT0006624 | 18185580 |
| Lung | hsa-let-7a-5p | MIMAT0000062 | 20005451 |
| Lung | hsa-let-7a-5p | MIMAT0000062 | 20033209 |
| Lung | hsa-let-7a-5p | MIMAT0000062 | 15766527 |
| Lung | hsa-let-7a-5p | MIMAT0000062 | 19818775 |
| Lung | hsa-let-7a-5p | MIMAT0000062 | 16651716 |
| Lung | hsa-let-7a-5p | MIMAT0000062 | 20418948 |
| Lung | hsa-let-7a-5p | MIMAT0000062 | 17942906 |
| Lung | hsa-let-7b-3p | MIMAT0004482 | 18379589 |
| Lung | hsa-let-7b-3p | MIMAT0004482 | 17437991 |
| Lung | hsa-let-7b-3p | MIMAT0004482 | 17699775 |
| Lung | hsa-let-7b-5p | MIMAT0000063 | 17942906 |
| Lung | hsa-let-7b-5p | MIMAT0000063 | 18379589 |
| Lung | hsa-let-7b-5p | MIMAT0000063 | 19966857 |
| Lung | hsa-let-7b-5p | MIMAT0000063 | 17437991 |
| Lung | hsa-let-7b-5p | MIMAT0000063 | 17699775 |
| Lung | hsa-let-7c-5p | MIMAT0000064 | 15766527 |
| Lung | hsa-let-7d-5p | MIMAT0000065 | 20395557 |
| Lung | hsa-let-7d-5p | MIMAT0000065 | 21616524 |
| Lung | hsa-let-7e-3p | MIMAT0004485 | 17437991 |
| Lung | hsa-let-7e-5p | MIMAT0000066 | 17437991 |
| Lung | hsa-let-7f-5p | MIMAT0000067 | 19956384 |
| Lung | hsa-let-7f-5p | MIMAT0000067 | 21616524 |
| Lung | hsa-let-7g-5p | MIMAT0000414 | 18308936 |
| Lung | hsa-let-7g-5p | MIMAT0000414 | 21616524 |
| Lung | hsa-let-7i-5p | MIMAT0000415 | 21616524 |
| Lung | hsa-miR-1 | MIMAT0000416 | 23142026 |
| Lung | hsa-miR-106a-5p | MIMAT0000103 | 16461460 |
| Lung | hsa-miR-107 | MIMAT0000104 | 19688090 |
| Lung | hsa-miR-125a-5p | MIMAT0000443 | 21220473 |
| Lung | hsa-miR-125b-5p | MIMAT0000423 | 19293287 |
| Lung | hsa-miR-126-3p | MIMAT0000445 | 19223090 |
| Lung | hsa-miR-126-3p | MIMAT0000445 | 18602365 |
| Lung | hsa-miR-126-3p | MIMAT0000445 | 20083669 |
| Lung | hsa-miR-128-2-5p | MIMAT0031095 | 18304967 |
| Lung | hsa-miR-133b | MIMAT0000770 | 19654003 |
| Lung | hsa-miR-134-5p | MIMAT0000447 | 20371173 |
| Lung | hsa-miR-137 | MIMAT0000429 | 23178712 |
| Lung | hsa-miR-143-3p | MIMAT0000435 | 19472311 |
| Lung | hsa-miR-145-5p | MIMAT0000437 | 21496429 |
| Lung | hsa-miR-145-5p | MIMAT0000437 | 21092188 |
| Lung | hsa-miR-145-5p | MIMAT0000437 | 19996288 |
| Lung | hsa-miR-146b-5p | MIMAT0002809 | 21305051 |
| Lung | hsa-miR-155-5p | MIMAT0000646 | 21093163 |
| Lung | hsa-miR-155-5p | MIMAT0000646 | 19701459 |
| Lung | hsa-miR-155-5p | MIMAT0000646 | 16675453 |
| Lung | hsa-miR-155-5p | MIMAT0000646 | 20427544 |
| Lung | hsa-miR-15a-5p | MIMAT0000068 | 19549910 |
| Lung | hsa-miR-16-5p | MIMAT0000069 | 19549910 |
| Lung | hsa-miR-183-5p | MIMAT0000261 | 18840437 |
| Lung | hsa-miR-185-5p | MIMAT0000455 | 19688090 |
| Lung | hsa-miR-188-5p | MIMAT0000457 | 19223510 |
| Lung | hsa-miR-196a-5p | MIMAT0000226 | 19418581 |
| Lung | hsa-miR-197-3p | MIMAT0000227 | 19671678 |
| Lung | hsa-miR-19a-3p | MIMAT0000073 | 21751348 |
| Lung | hsa-miR-200b-3p | MIMAT0000318 | 21993663 |
| Lung | hsa-miR-200b-3p | MIMAT0000318 | 22139708 |
| Lung | hsa-miR-200c-3p | MIMAT0000617 | 21207483 |
| Lung | hsa-miR-200c-3p | MIMAT0000617 | 19223510 |
| Lung | hsa-miR-200c-3p | MIMAT0000617 | 21993663 |
| Lung | hsa-miR-200c-3p | MIMAT0000617 | 21294122 |
| Lung | hsa-miR-204-5p | MIMAT0000265 | 20369013 |
| Lung | hsa-miR-20a-5p | MIMAT0000075 | 21751348 |
| Lung | hsa-miR-20a-5p | MIMAT0000075 | 18632605 |
| Lung | hsa-miR-20a-5p | MIMAT0000075 | 16461460 |
| Lung | hsa-miR-21-5p | MIMAT0000076 | 18270520 |
| Lung | hsa-miR-21-5p | MIMAT0000076 | 22956424 |
| Lung | hsa-miR-21-5p | MIMAT0000076 | 18372920 |
| Lung | hsa-miR-21-5p | MIMAT0000076 | 17968323 |
| Lung | hsa-miR-21-5p | MIMAT0000076 | 20223231 |
| Lung | hsa-miR-22-3p | MIMAT0000077 | 22484852 |
| Lung | hsa-miR-221-3p | MIMAT0000278 | 18246122 |
| Lung | hsa-miR-222-3p | MIMAT0000279 | 21656127 |
| Lung | hsa-miR-222-3p | MIMAT0000279 | 18246122 |
| Lung | hsa-miR-223-3p | MIMAT0000280 | 18278031 |
| Lung | hsa-miR-223-3p | MIMAT0000280 | 21305051 |
| Lung | hsa-miR-26a-5p | MIMAT0000082 | 16461460 |
| Lung | hsa-miR-29a-3p | MIMAT0000086 | 19818597 |
| Lung | hsa-miR-29a-3p | MIMAT0000086 | 17890317 |
| Lung | hsa-miR-29b-3p | MIMAT0000100 | 21305051 |
| Lung | hsa-miR-29b-3p | MIMAT0000100 | 17890317 |
| Lung | hsa-miR-29c-3p | MIMAT0000681 | 21305051 |
| Lung | hsa-miR-29c-3p | MIMAT0000681 | 17890317 |
| Lung | hsa-miR-30c-5p | MIMAT0000244 | 19223510 |
| Lung | hsa-miR-30e-5p | MIMAT0000692 | 19223510 |
| Lung | hsa-miR-31-3p | MIMAT0004504 | 20237410 |
| Lung | hsa-miR-31-5p | MIMAT0000089 | 21048943 |
| Lung | hsa-miR-31-5p | MIMAT0000089 | 20237410 |
| Lung | hsa-miR-31-5p | MIMAT0000089 | 23539435 |
| Lung | hsa-miR-335-5p | MIMAT0000765 | 18185580 |
| Lung | hsa-miR-34a-5p | MIMAT0000255 | 21294122 |
| Lung | hsa-miR-409-3p | MIMAT0001639 | 22531314 |
| Lung | hsa-miR-429 | MIMAT0001536 | 21993663 |
| Lung | hsa-miR-449c-5p | MIMAT0010251 | 23507140 |
| Lung | hsa-miR-451a | MIMAT0001631 | 21358675 |
| Lung | hsa-miR-483-3p | MIMAT0002173 | 21305051 |
| Lung | hsa-miR-499a-5p | MIMAT0002870 | 21934092 |
| Lung | hsa-miR-499b-5p | MIMAT0019897 | 21934092 |
| Lung | hsa-miR-574-5p | MIMAT0004795 | 21305051 |
| Lung | hsa-miR-622 | MIMAT0003291 | 22016468 |
| Lung | hsa-miR-7-5p | MIMAT0000252 | 19073608 |
| Lung | hsa-miR-93-5p | MIMAT0000093 | 19671678 |
| Lung | hsa-miR-98-5p | MIMAT0000096 | 19671678 |
| Lung | hsa-miR-98-5p | MIMAT0000096 | 21616524 |
| Lung | hsa-miR-98-5p | MIMAT0000096 | 17942906 |
| Lung | mmu-let-7a-5p | MIMAT0000521 | 20630862 |
| Lung | mmu-miR-106b-5p | MIMAT0000386 | 19559694 |
| Lung | mmu-miR-10b-5p | MIMAT0000208 | 20351690 |
| Lung | mmu-miR-122-5p | MIMAT0000246 | 16258535 |
| Lung | mmu-miR-127-3p | MIMAT0000139 | 22287715 |
| Lung | mmu-miR-135b-5p | MIMAT0000612 | 23440414 |
| Lung | mmu-miR-155-5p | MIMAT0000165 | 19701459 |
| Lung | mmu-miR-155-5p | MIMAT0000165 | 17463290 |
| Lung | mmu-miR-17-5p | MIMAT0000649 | 19559694 |
| Lung | mmu-miR-17-5p | MIMAT0000649 | 17765889 |
| Lung | mmu-miR-200a-3p | MIMAT0000519 | 21115742 |
| Lung | mmu-miR-200b-3p | MIMAT0000233 | 21115742 |
| Lung | mmu-miR-200b-3p | MIMAT0000233 | 19787069 |
| Lung | mmu-miR-200c-3p | MIMAT0000657 | 21115742 |
| Lung | mmu-miR-200c-3p | MIMAT0000657 | 19787069 |
| Lung | mmu-miR-204-5p | MIMAT0000237 | 20369013 |
| Lung | mmu-miR-20a-5p | MIMAT0000529 | 19559694 |
| Lung | mmu-miR-21a-5p | MIMAT0000530 | 18372920 |
| Lung | mmu-miR-21a-5p | MIMAT0000530 | 19748927 |
| Lung | mmu-miR-223-3p | MIMAT0000665 | 18278031 |
| Lung | mmu-miR-31-5p | MIMAT0000538 | 20237410 |
| Lung | mmu-miR-429-3p | MIMAT0001537 | 21115742 |
| Lung | ppy-miR-335 | MIMAT0015837 | 18185580 |
| Lung | ptr-miR-335 | MIMAT0008104 | 18185580 |
| Lung | rno-let-7a-5p | MIMAT0000774 | 17942906 |
| Lung | rno-miR-126a-3p | MIMAT0000832 | 18780894 |
| Lung | rno-miR-126a-5p | MIMAT0000831 | 18780894 |
| Lung | rno-miR-335 | MIMAT0000575 | 18185580 |
| Lung | ssc-miR-335 | MIMAT0013955 | 18185580 |
| Lymph | hsa-miR-101-3p | MIMAT0000099 | 19043531 |
| Lymph | hsa-miR-135a-5p | MIMAT0000428 | 19666866 |
| Lymph | hsa-miR-138-5p | MIMAT0000430 | 23389731 |
| Lymph | hsa-miR-143-3p | MIMAT0000435 | 23276710 |
| Lymph | hsa-miR-148a-3p | MIMAT0000243 | 18768788 |
| Lymph | hsa-miR-16-2-3p | MIMAT0004518 | 20309880 |
| Lymph | hsa-miR-181a-5p | MIMAT0000256 | 20841506 |
| Lymph | hsa-miR-200c-3p | MIMAT0000617 | 21207483 |
| Lymph | hsa-miR-200c-3p | MIMAT0000617 | 21294122 |
| Lymph | hsa-miR-21-5p | MIMAT0000076 | 19276261 |
| Lymph | hsa-miR-21-5p | MIMAT0000076 | 20346171 |
| Lymph | hsa-miR-21-5p | MIMAT0000076 | 22267008 |
| Lymph | hsa-miR-21-5p | MIMAT0000076 | 22956424 |
| Lymph | hsa-miR-223-3p | MIMAT0000280 | 22270966 |
| Lymph | hsa-miR-26a-5p | MIMAT0000082 | 23338972 |
| Lymph | hsa-miR-339-5p | MIMAT0000764 | 20932331 |
| Lymph | hsa-miR-34a-5p | MIMAT0000255 | 21294122 |
| Lymph | hsa-miR-34a-5p | MIMAT0000255 | 20309880 |
| Lymph | hsa-miR-34b-3p | MIMAT0004676 | 18768788 |
| Lymph | hsa-miR-34c-5p | MIMAT0000686 | 18768788 |
| Lymph | hsa-miR-497-5p | MIMAT0002820 | 23453369 |
| Lymph | hsa-miR-499a-5p | MIMAT0002870 | 21934092 |
| Lymph | hsa-miR-499b-5p | MIMAT0019897 | 21934092 |
| Lymph Nodes | hsa-miR-101-3p | MIMAT0000099 | 19043531 |
| Lymph Nodes | hsa-miR-148a-3p | MIMAT0000243 | 18768788 |
| Lymph Nodes | hsa-miR-16-2-3p | MIMAT0004518 | 20309880 |
| Lymph Nodes | hsa-miR-21-5p | MIMAT0000076 | 22267008 |
| Lymph Nodes | hsa-miR-34a-5p | MIMAT0000255 | 20309880 |
| Lymph Nodes | hsa-miR-34b-3p | MIMAT0004676 | 18768788 |
| Lymph Nodes | hsa-miR-34c-5p | MIMAT0000686 | 18768788 |
| Lymphocytes | hsa-let-7d-5p | MIMAT0000065 | 21616524 |
| Lymphocytes | hsa-let-7f-5p | MIMAT0000067 | 21616524 |
| Lymphocytes | hsa-let-7g-5p | MIMAT0000414 | 21616524 |
| Lymphocytes | hsa-let-7i-5p | MIMAT0000415 | 21616524 |
| Lymphocytes | hsa-miR-101-3p | MIMAT0000099 | 19043531 |
| Lymphocytes | hsa-miR-107 | MIMAT0000104 | 20884628 |
| Lymphocytes | hsa-miR-122-5p | MIMAT0000421 | 20884628 |
| Lymphocytes | hsa-miR-125b-5p | MIMAT0000423 | 20497960 |
| Lymphocytes | hsa-miR-146a-5p | MIMAT0000449 | 18057241 |
| Lymphocytes | hsa-miR-146a-5p | MIMAT0000449 | 19965651 |
| Lymphocytes | hsa-miR-148a-3p | MIMAT0000243 | 19435428 |
| Lymphocytes | hsa-miR-150-5p | MIMAT0000451 | 17923094 |
| Lymphocytes | hsa-miR-150-5p | MIMAT0000451 | 18667440 |
| Lymphocytes | hsa-miR-155-5p | MIMAT0000646 | 20209161 |
| Lymphocytes | hsa-miR-155-5p | MIMAT0000646 | 20680360 |
| Lymphocytes | hsa-miR-155-5p | MIMAT0000646 | 19386588 |
| Lymphocytes | hsa-miR-155-5p | MIMAT0000646 | 18367535 |
| Lymphocytes | hsa-miR-15a-5p | MIMAT0000068 | 22379033 |
| Lymphocytes | hsa-miR-15b-5p | MIMAT0000417 | 22379033 |
| Lymphocytes | hsa-miR-16-5p | MIMAT0000069 | 22379033 |
| Lymphocytes | hsa-miR-181c-5p | MIMAT0000258 | 21112091 |
| Lymphocytes | hsa-miR-198 | MIMAT0000228 | 18667440 |
| Lymphocytes | hsa-miR-19b-3p | MIMAT0000074 | 19435428 |
| Lymphocytes | hsa-miR-20a-5p | MIMAT0000075 | 19435428 |
| Lymphocytes | hsa-miR-214-3p | MIMAT0000271 | 20548023 |
| Lymphocytes | hsa-miR-214-3p | MIMAT0000271 | 19435428 |
| Lymphocytes | hsa-miR-221-3p | MIMAT0000278 | 21236259 |
| Lymphocytes | hsa-miR-221-3p | MIMAT0000278 | 20110463 |
| Lymphocytes | hsa-miR-26a-5p | MIMAT0000082 | 23338972 |
| Lymphocytes | hsa-miR-29a-3p | MIMAT0000086 | 20086245 |
| Lymphocytes | hsa-miR-29b-3p | MIMAT0000100 | 20086245 |
| Lymphocytes | hsa-miR-29c-3p | MIMAT0000681 | 20086245 |
| Lymphocytes | hsa-miR-34a-5p | MIMAT0000255 | 20598588 |
| Lymphocytes | hsa-miR-520h | MIMAT0002867 | 19435428 |
| Lymphocytes | hsa-miR-92a-3p | MIMAT0000092 | 19435428 |
| Lymphocytes | hsa-miR-98-5p | MIMAT0000096 | 21616524 |
| Lymphocytes | mmu-miR-125b-5p | MIMAT0000136 | 21118986 |
| Lymphocytes | mmu-miR-146a-5p | MIMAT0000158 | 18791161 |
| Lymphocytes | mmu-miR-148a-3p | MIMAT0000516 | 21068402 |
| Lymphocytes | mmu-miR-148b-3p | MIMAT0000580 | 21068402 |
| Lymphocytes | mmu-miR-150-5p | MIMAT0000160 | 17923094 |
| Lymphocytes | mmu-miR-152-3p | MIMAT0000162 | 21068402 |
| Lymphocytes | mmu-miR-155-5p | MIMAT0000165 | 18450484 |
| Lymphocytes | mmu-miR-155-5p | MIMAT0000165 | 18455451 |
| Lymphocytes | mmu-miR-155-5p | MIMAT0000165 | 17463290 |
| Lymphocytes | mmu-miR-214-3p | MIMAT0000661 | 20548023 |
| Lymphocytes | mmu-miR-223-3p | MIMAT0000665 | 20935160 |
| Lymphocytes | mmu-miR-223-3p | MIMAT0000665 | 22043014 |
| Lymphocytes | mmu-miR-34a-5p | MIMAT0000542 | 20598588 |
| Lymphocytes Null | hsa-miR-1 | MIMAT0000416 | 18593903 |
| Lymphoid Tissue | hsa-miR-101-3p | MIMAT0000099 | 19043531 |
| Lymphoid Tissue | hsa-miR-16-5p | MIMAT0000069 | 17351108 |
| Lymphoid Tissue | hsa-miR-200a-3p | MIMAT0000682 | 22183793 |
| Lymphoid Tissue | hsa-miR-200b-3p | MIMAT0000318 | 22183793 |
| Lymphoid Tissue | hsa-miR-200c-3p | MIMAT0000617 | 22183793 |
| Lymphoid Tissue | hsa-miR-7-5p | MIMAT0000252 | 18823940 |
| Macrophages | hsa-let-7b-5p | MIMAT0000063 | 20130213 |
| Macrophages | hsa-miR-101-3p | MIMAT0000099 | 21068409 |
| Macrophages | hsa-miR-141-3p | MIMAT0000432 | 18835392 |
| Macrophages | hsa-miR-145-5p | MIMAT0000437 | 20130213 |
| Macrophages | hsa-miR-146a-5p | MIMAT0000449 | 20542134 |
| Macrophages | hsa-miR-155-5p | MIMAT0000646 | 21036908 |
| Macrophages | hsa-miR-155-5p | MIMAT0000646 | 19359473 |
| Macrophages | hsa-miR-155-5p | MIMAT0000646 | 19386588 |
| Macrophages | hsa-miR-155-5p | MIMAT0000646 | 19887047 |
| Macrophages | hsa-miR-155-5p | MIMAT0000646 | 21097505 |
| Macrophages | hsa-miR-155-5p | MIMAT0000646 | 19596814 |
| Macrophages | hsa-miR-155-5p | MIMAT0000646 | 21030878 |
| Macrophages | hsa-miR-155-5p | MIMAT0000646 | 22473996 |
| Macrophages | hsa-miR-192-5p | MIMAT0000222 | 18835392 |
| Macrophages | hsa-miR-195-5p | MIMAT0000461 | 20952681 |
| Macrophages | hsa-miR-198 | MIMAT0000228 | 19148268 |
| Macrophages | hsa-miR-200a-3p | MIMAT0000682 | 18835392 |
| Macrophages | hsa-miR-203a | MIMAT0000264 | 23522925 |
| Macrophages | hsa-miR-21-5p | MIMAT0000076 | 18384814 |
| Macrophages | hsa-miR-217 | MIMAT0000274 | 18835392 |
| Macrophages | hsa-miR-26a-5p | MIMAT0000082 | 20130213 |
| Macrophages | hsa-miR-27b-3p | MIMAT0000419 | 20164187 |
| Macrophages | hsa-miR-27b-3p | MIMAT0000419 | 18835392 |
| Macrophages | hsa-miR-33a-5p | MIMAT0000091 | 20466885 |
| Macrophages | hsa-miR-34a-5p | MIMAT0000255 | 20130213 |
| Macrophages | hsa-miR-451a | MIMAT0001631 | 19318487 |
| Macrophages | hsa-miR-532-5p | MIMAT0002888 | 18835392 |
| Macrophages | hsa-miR-603 | MIMAT0003271 | 18835392 |
| Macrophages | hsa-miR-758-3p | MIMAT0003879 | 21885853 |
| Macrophages | hsa-miR-769-5p | MIMAT0003886 | 18835392 |
| Macrophages | mmu-let-7e-3p | MIMAT0017016 | 19699171 |
| Macrophages | mmu-let-7e-5p | MIMAT0000524 | 19699171 |
| Macrophages | mmu-miR-101a-3p | MIMAT0000133 | 21068409 |
| Macrophages | mmu-miR-122-5p | MIMAT0000246 | 21364282 |
| Macrophages | mmu-miR-124-3p | MIMAT0000134 | 21131957 |
| Macrophages | mmu-miR-127-3p | MIMAT0000139 | 22287715 |
| Macrophages | mmu-miR-146a-5p | MIMAT0000158 | 19596990 |
| Macrophages | mmu-miR-155-5p | MIMAT0000165 | 20937844 |
| Macrophages | mmu-miR-155-5p | MIMAT0000165 | 19699171 |
| Macrophages | mmu-miR-21a-5p | MIMAT0000530 | 21273303 |
| Macrophages | mmu-miR-33-5p | MIMAT0000667 | 20466885 |
| Macrophages | mmu-miR-33-5p | MIMAT0000667 | 21285396 |
| Macrophages | mmu-miR-466l-3p | MIMAT0005830 | 20410487 |
| Macrophages Peritoneal | hsa-miR-758-3p | MIMAT0003879 | 21885853 |
| Macrophages Peritoneal | mmu-miR-33-5p | MIMAT0000667 | 21285396 |
| Mammary Arteries | hsa-miR-155-5p | MIMAT0000646 | 23108656 |
| Mammary Glands Human | hsa-miR-1258 | MIMAT0005909 | 21266359 |
| Mammary Glands Human | hsa-miR-155-5p | MIMAT0000646 | 18794355 |
| Mammary Glands Human | mmu-miR-101a-3p | MIMAT0000133 | 19281778 |
| Mammary Glands Human | mmu-miR-132-3p | MIMAT0000144 | 21057503 |
| Mammary Glands Human | mmu-miR-132-5p | MIMAT0016984 | 21057503 |
| Mammary Glands Human | mmu-miR-155-5p | MIMAT0000165 | 18794355 |
| Mammary Glands Human | mmu-miR-212-3p | MIMAT0000659 | 21057503 |
| Mast Cells | hsa-miR-132-3p | MIMAT0000426 | 21853268 |
| Maxillary Sinus | hsa-miR-1 | MIMAT0000416 | 21701775 |
| Maxillary Sinus | hsa-miR-133a-3p | MIMAT0000427 | 21701775 |
| Megakaryocyte-Erythroid | hsa-miR-150-5p | MIMAT0000451 | 18539114 |
| Progenitor Cells | | | |
| Megakaryocytes | hsa-let-7a-5p | MIMAT0000062 | 20445018 |
| Megakaryocytes | hsa-let-7c-5p | MIMAT0000064 | 20445018 |
| Megakaryocytes | hsa-let-7d-5p | MIMAT0000065 | 20445018 |
| Megakaryocytes | hsa-let-7e-5p | MIMAT0000066 | 20445018 |
| Megakaryocytes | hsa-let-7f-5p | MIMAT0000067 | 20445018 |
| Megakaryocytes | hsa-miR-10a-5p | MIMAT0000253 | 16549775 |
| Megakaryocytes | hsa-miR-130a-3p | MIMAT0000425 | 16549775 |
| Megakaryocytes | hsa-miR-150-5p | MIMAT0000451 | 18539114 |
| Megakaryocytes | hsa-miR-151a-5p | MIMAT0004697 | 20445018 |
| Megakaryocytes | hsa-miR-28-5p | MIMAT0000085 | 20445018 |
| Megakaryocytes | hsa-miR-708-3p | MIMAT0004927 | 20445018 |
| Megakaryocytes | hsa-miR-98-5p | MIMAT0000096 | 20445018 |
| Melanocytes | hsa-let-7a-5p | MIMAT0000062 | 18679415 |
| Melanocytes | hsa-miR-137 | MIMAT0000429 | 21051724 |
| Melanocytes | hsa-miR-155-5p | MIMAT0000646 | 21466664 |
| Melanocytes | hsa-miR-196a-5p | MIMAT0000226 | 20480203 |
| Melanocytes | hsa-miR-196a-5p | MIMAT0000226 | 21077158 |
| Melanocytes | hsa-miR-211-5p | MIMAT0000268 | 21072171 |
| Melanocytes | hsa-miR-211-5p | MIMAT0000268 | 21435193 |
| Melanocytes | hsa-miR-221-3p | MIMAT0000278 | 23400877 |
| Melanocytes | hsa-miR-222-3p | MIMAT0000279 | 23400877 |
| Melanocytes | hsa-miR-34a-5p | MIMAT0000255 | 19029026 |
| Melanocytes | hsa-miR-532-5p | MIMAT0002888 | 19336521 |
| Melanocytes | hsa-miR-7-5p | MIMAT0000252 | 19029026 |
| Membranes | hsa-miR-1 | MIMAT0000416 | 17401374 |
| Membranes | hsa-miR-128-3p | MIMAT0000424 | 21143953 |
| Membranes | hsa-miR-146a-5p | MIMAT0000449 | 18057241 |
| Membranes | hsa-miR-151a-3p | MIMAT0000757 | 21143953 |
| Membranes | hsa-miR-155-5p | MIMAT0000646 | 19386588 |
| Membranes | hsa-miR-155-5p | MIMAT0000646 | 22387553 |
| Membranes | hsa-miR-15a-5p | MIMAT0000068 | 20385127 |
| Membranes | hsa-miR-182-5p | MIMAT0000259 | 23430586 |
| Membranes | hsa-miR-185-5p | MIMAT0000455 | 21143953 |
| Membranes | hsa-miR-204-5p | MIMAT0000265 | 20056717 |
| Membranes | hsa-miR-21-5p | MIMAT0000076 | 22387553 |
| Membranes | hsa-miR-21-5p | MIMAT0000076 | 20056717 |
| Membranes | hsa-miR-210-3p | MIMAT0000267 | 19826008 |
| Membranes | hsa-miR-29a-3p | MIMAT0000086 | 19951903 |
| Membranes | hsa-miR-30b-5p | MIMAT0000420 | 22387553 |
| Membranes | hsa-miR-320a | MIMAT0000510 | 20628061 |
| Membranes | hsa-miR-338-3p | MIMAT0000763 | 18991333 |
| Membranes | hsa-miR-509-3p | MIMAT0002881 | 21143953 |
| Membranes | hsa-miR-92b-3p | MIMAT0003218 | 21030610 |
| Membranes | mmu-miR-143-3p | MIMAT0000247 | 20351064 |
| Membranes | mmu-miR-145a-5p | MIMAT0000157 | 20351064 |
| Membranes | mmu-miR-200b-3p | MIMAT0000233 | 19787069 |
| Membranes | mmu-miR-200c-3p | MIMAT0000657 | 19787069 |
| Membranes | mmu-miR-23a-3p | MIMAT0000532 | 19259393 |
| Membranes | mmu-miR-23b-3p | MIMAT0000125 | 19259393 |
| Membranes | rno-miR-1-3p | MIMAT0003125 | 17401374 |
| Membranes | rno-miR-1-3p | MIMAT0003125 | 18801338 |
| Membranes | rno-miR-338-3p | MIMAT0000581 | 18991333 |
| Meninges | hsa-miR-200a-3p | MIMAT0000682 | 19703993 |
| Mesangial Cells | hsa-miR-328-3p | MIMAT0000752 | 18716028 |
| Mesangial Cells | hsa-miR-377-3p | MIMAT0000730 | 18716028 |
| Mesangial Cells | mmu-miR-192-5p | MIMAT0000517 | 17360662 |
| Mesangial Cells | mmu-miR-215-5p | MIMAT0000904 | 17360662 |
| Mesangial Cells | mmu-miR-216a-5p | MIMAT0000662 | 20713358 |
| Mesangial Cells | mmu-miR-216a-5p | MIMAT0000662 | 19543271 |
| Mesangial Cells | mmu-miR-217-5p | MIMAT0000679 | 19543271 |
| Mesangial Cells | rno-miR-192-5p | MIMAT0000867 | 20393144 |
| Mesangial Cells | rno-miR-215 | MIMAT0003118 | 20393144 |
| Mesangial Cells | rno-miR-25-3p | MIMAT0000795 | 21071935 |
| Mesangial Cells | rno-miR-92b-3p | MIMAT0005340 | 21071935 |
| Mesencephalon | hsa-miR-133b | MIMAT0000770 | 17761882 |
| Mesenchymal Stem Cells | bta-miR-130a | MIMAT0009223 | 17855557 |
| Mesenchymal Stem Cells | cfa-miR-130a | MIMAT0006631 | 17855557 |
| Mesenchymal Stem Cells | dre-miR-130a | MIMAT0001826 | 17855557 |
| Mesenchymal Stem Cells | hsa-miR-100-5p | MIMAT0000098 | 22684006 |
| Mesenchymal Stem Cells | hsa-miR-125b-5p | MIMAT0000423 | 18230348 |
| Mesenchymal Stem Cells | hsa-miR-130a-3p | MIMAT0000425 | 17855557 |
| Mesenchymal Stem Cells | hsa-miR-133a-3p | MIMAT0000427 | 23069713 |
| Mesenchymal Stem Cells | hsa-miR-138-5p | MIMAT0000430 | 20486779 |
| Mesenchymal Stem Cells | hsa-miR-145-5p | MIMAT0000437 | 20382729 |
| Mesenchymal Stem Cells | hsa-miR-145-5p | MIMAT0000437 | 21217773 |
| Mesenchymal Stem Cells | hsa-miR-196a-5p | MIMAT0000226 | 19063684 |
| Mesenchymal Stem Cells | hsa-miR-199a-5p | MIMAT0000231 | 22441842 |
| Mesenchymal Stem Cells | hsa-miR-206 | MIMAT0000462 | 17855557 |
| Mesenchymal Stem Cells | hsa-miR-21-5p | MIMAT0000076 | 21381024 |
| Mesenchymal Stem Cells | hsa-miR-21-5p | MIMAT0000076 | 19816956 |
| Mesenchymal Stem Cells | hsa-miR-21-5p | MIMAT0000076 | 23239100 |
| Mesenchymal Stem Cells | hsa-miR-27a-3p | MIMAT0000084 | 19440384 |
| Mesenchymal Stem Cells | hsa-miR-29a-3p | MIMAT0000086 | 21665270 |
| Mesenchymal Stem Cells | hsa-miR-29b-3p | MIMAT0000100 | 21665270 |
| Mesenchymal Stem Cells | hsa-miR-302a-3p | MIMAT0000684 | 17855557 |
| Mesenchymal Stem Cells | hsa-miR-320a | MIMAT0000510 | 17855557 |
| Mesenchymal Stem Cells | hsa-miR-335-5p | MIMAT0000765 | 21164520 |
| Mesenchymal Stem Cells | hsa-miR-433-3p | MIMAT0001627 | 23353875 |
| Mesenchymal Stem Cells | hsa-miR-489-3p | MIMAT0002805 | 19440384 |
| Mesenchymal Stem Cells | hsa-miR-637 | MIMAT0003307 | 21880893 |
| Mesenchymal Stem Cells | mmu-miR-204-5p | MIMAT0000237 | 20039258 |
| Mesenchymal Stem Cells | mmu-miR-211-5p | MIMAT0000668 | 20039258 |
| Mesenchymal Stem Cells | mmu-miR-29a-3p | MIMAT0000535 | 21665270 |
| Mesenchymal Stem Cells | mmu-miR-29b-3p | MIMAT0000127 | 21665270 |
| Mesenchymal Stem Cells | mmu-miR-9-5p | MIMAT0000142 | 22465325 |
| Mesenchymal Stem Cells | ppy-miR-130a | MIMAT0015753 | 17855557 |
| Mesenchymal Stem Cells | ptr-miR-130a | MIMAT0008025 | 17855557 |
| Mesenchymal Stem Cells | ssc-miR-130a | MIMAT0007758 | 17855557 |
| Mesenchymal Stem Cells | xtr-miR-130a | MIMAT0003591 | 17855557 |
| Mesenteric Arteries | mmu-miR-130a-3p | MIMAT0000141 | 21753805 |
| Mesenteric Artery Superior | mmu-miR-130a-3p | MIMAT0000141 | 21753805 |
| Mesoderm | hsa-miR-155-5p | MIMAT0000646 | 19701459 |
| Mesoderm | mmu-miR-137-3p | MIMAT0000149 | 21256124 |
| Mesoderm | mmu-miR-153-3p | MIMAT0000163 | 21256124 |
| Mesoderm | mmu-miR-155-5p | MIMAT0000165 | 19701459 |
| Mesoderm | mmu-miR-21a-5p | MIMAT0000530 | 21295561 |
| Mesoderm | mmu-miR-290a-3p | MIMAT0004572 | 19628328 |
| Mesoderm | mmu-miR-291a-3p | MIMAT0000368 | 19628328 |
| Mesoderm | mmu-miR-292-3p | MIMAT0000370 | 19628328 |
| Mesoderm | mmu-miR-293-3p | MIMAT0000371 | 19628328 |
| Mesoderm | mmu-miR-294-3p | MIMAT0000372 | 19628328 |
| Mesoderm | mmu-miR-295-3p | MIMAT0000373 | 19628328 |
| Mesoderm | mmu-miR-363-3p | MIMAT0000708 | 21256124 |
| Microfilaments | hsa-miR-1 | MIMAT0000416 | 23142026 |
| Microfilaments | mmu-miR-124-3p | MIMAT0000134 | 18619591 |
| Microglia | hsa-miR-146a-5p | MIMAT0000449 | 20181935 |
| Microglia | mmu-miR-124-3p | MIMAT0000134 | 21131957 |
| Microglia | mmu-miR-155-5p | MIMAT0000165 | 22043967 |
| Microtubules | mmu-miR-124-3p | MIMAT0000134 | 18619591 |
| Microvessels | hsa-miR-135b-5p | MIMAT0000758 | 23340180 |
| Microvessels | hsa-miR-223-3p | MIMAT0000280 | 23208072 |
| Microvessels | hsa-miR-29b-3p | MIMAT0000100 | 21793034 |
| Microvessels | mmu-miR-15a-5p | MIMAT0000526 | 20445066 |
| Middle Cerebral Artery | mmu-miR-497-5p | MIMAT0003453 | 20053374 |
| Middle Cerebral Artery | rno-let-7a-5p | MIMAT0000774 | 18258830 |
| Middle Cerebral Artery | rno-let-7b-3p | MIMAT0004705 | 18258830 |
| Middle Cerebral Artery | rno-let-7b-5p | MIMAT0000775 | 18258830 |
| Middle Cerebral Artery | rno-let-7c-5p | MIMAT0000776 | 18258830 |
| Middle Cerebral Artery | rno-let-7d-3p | MIMAT0000563 | 18258830 |
| Middle Cerebral Artery | rno-let-7d-5p | MIMAT0000562 | 18258830 |
| Middle Cerebral Artery | rno-let-7e-3p | MIMAT0004706 | 18258830 |
| Middle Cerebral Artery | rno-let-7e-5p | MIMAT0000777 | 18258830 |
| Middle Cerebral Artery | rno-let-7f-5p | MIMAT0000778 | 18258830 |
| Middle Cerebral Artery | rno-let-7i-3p | MIMAT0004707 | 18258830 |
| Middle Cerebral Artery | rno-let-7i-5p | MIMAT0000779 | 18258830 |
| Middle Cerebral Artery | rno-miR-100-5p | MIMAT0000822 | 18258830 |
| Middle Cerebral Artery | rno-miR-101a-3p | MIMAT0000823 | 18258830 |
| Middle Cerebral Artery | rno-miR-101b-3p | MIMAT0000615 | 18258830 |
| Middle Cerebral Artery | rno-miR-124-3p | MIMAT0000828 | 18258830 |
| Middle Cerebral Artery | rno-miR-125a-5p | MIMAT0000829 | 18258830 |
| Middle Cerebral Artery | rno-miR-129-5p | MIMAT0000600 | 18258830 |
| Middle Cerebral Artery | rno-miR-130a-3p | MIMAT0000836 | 18258830 |
| Middle Cerebral Artery | rno-miR-132-3p | MIMAT0000838 | 18258830 |
| Middle Cerebral Artery | rno-miR-134-5p | MIMAT0000840 | 18258830 |
| Middle Cerebral Artery | rno-miR-138-5p | MIMAT0000844 | 18258830 |
| Middle Cerebral Artery | rno-miR-139-5p | MIMAT0000845 | 18258830 |
| Middle Cerebral Artery | rno-miR-145-5p | MIMAT0000851 | 19142192 |
| Middle Cerebral Artery | rno-miR-150-5p | MIMAT0000853 | 18258830 |
| Middle Cerebral Artery | rno-miR-181b-5p | MIMAT0000859 | 18258830 |
| Middle Cerebral Artery | rno-miR-185-5p | MIMAT0000862 | 18258830 |
| Middle Cerebral Artery | rno-miR-204-5p | MIMAT0000877 | 18258830 |
| Middle Cerebral Artery | rno-miR-21-5p | MIMAT0000790 | 18258830 |
| Middle Cerebral Artery | rno-miR-214-3p | MIMAT0000885 | 18258830 |
| Middle Cerebral Artery | rno-miR-215 | MIMAT0003118 | 18258830 |
| Middle Cerebral Artery | rno-miR-218a-5p | MIMAT0000888 | 18258830 |
| Middle Cerebral Artery | rno-miR-223-3p | MIMAT0000892 | 18258830 |
| Middle Cerebral Artery | rno-miR-23a-3p | MIMAT0000792 | 18258830 |
| Middle Cerebral Artery | rno-miR-23b-3p | MIMAT0000793 | 18258830 |
| Middle Cerebral Artery | rno-miR-24-3p | MIMAT0000794 | 18258830 |
| Middle Cerebral Artery | rno-miR-25-3p | MIMAT0000795 | 18258830 |
| Middle Cerebral Artery | rno-miR-26b-5p | MIMAT0000797 | 18258830 |
| Middle Cerebral Artery | rno-miR-27a-3p | MIMAT0000799 | 18258830 |
| Middle Cerebral Artery | rno-miR-290 | MIMAT0000893 | 18258830 |
| Middle Cerebral Artery | rno-miR-292-5p | MIMAT0000896 | 18258830 |
| Middle Cerebral Artery | rno-miR-293-5p | MIMAT0012847 | 18258830 |
| Middle Cerebral Artery | rno-miR-298-5p | MIMAT0000900 | 18258830 |
| Middle Cerebral Artery | rno-miR-29a-3p | MIMAT0000802 | 18258830 |
| Middle Cerebral Artery | rno-miR-29b-3p | MIMAT0000801 | 18258830 |
| Middle Cerebral Artery | rno-miR-29c-3p | MIMAT0000803 | 18258830 |
| Middle Cerebral Artery | rno-miR-301a-3p | MIMAT0000552 | 18258830 |
| Middle Cerebral Artery | rno-miR-30a-3p | MIMAT0000809 | 18258830 |
| Middle Cerebral Artery | rno-miR-30a-5p | MIMAT0000808 | 18258830 |
| Middle Cerebral Artery | rno-miR-30b-5p | MIMAT0000806 | 18258830 |
| Middle Cerebral Artery | rno-miR-30c-5p | MIMAT0000804 | 18258830 |
| Middle Cerebral Artery | rno-miR-30e-5p | MIMAT0000805 | 18258830 |
| Middle Cerebral Artery | rno-miR-320-3p | MIMAT0000903 | 18258830 |
| Middle Cerebral Artery | rno-miR-322-5p | MIMAT0001619 | 18258830 |
| Middle Cerebral Artery | rno-miR-324-5p | MIMAT0000553 | 18258830 |
| Middle Cerebral Artery | rno-miR-327 | MIMAT0000561 | 18258830 |
| Middle Cerebral Artery | rno-miR-328a-3p | MIMAT0000564 | 18258830 |
| Middle Cerebral Artery | rno-miR-331-3p | MIMAT0000570 | 18258830 |
| Middle Cerebral Artery | rno-miR-338-3p | MIMAT0000581 | 18258830 |
| Middle Cerebral Artery | rno-miR-342-3p | MIMAT0000589 | 18258830 |
| Middle Cerebral Artery | rno-miR-342-5p | MIMAT0004652 | 18258830 |
| Middle Cerebral Artery | rno-miR-34a-5p | MIMAT0000815 | 18258830 |
| Middle Cerebral Artery | rno-miR-374-5p | MIMAT0003208 | 18258830 |
| Middle Cerebral Artery | rno-miR-382-5p | MIMAT0003201 | 18258830 |
| Middle Cerebral Artery | rno-miR-383-5p | MIMAT0003114 | 18258830 |
| Middle Cerebral Artery | rno-miR-409a-3p | MIMAT0003205 | 18258830 |
| Middle Cerebral Artery | rno-miR-485-5p | MIMAT0003203 | 18258830 |
| Middle Cerebral Artery | rno-miR-494-3p | MIMAT0003193 | 18258830 |
| Middle Cerebral Artery | rno-miR-497-5p | MIMAT0003383 | 18258830 |
| Middle Cerebral Artery | rno-miR-543-5p | MIMAT0004787 | 18258830 |
| Middle Cerebral Artery | rno-miR-664-3p | MIMAT0003382 | 18258830 |
| Middle Cerebral Artery | rno-miR-7a-5p | MIMAT0000606 | 18258830 |
| Middle Cerebral Artery | rno-miR-92a-3p | MIMAT0000816 | 18258830 |
| Middle Cerebral Artery | rno-miR-98-5p | MIMAT0000819 | 18258830 |
| Middle Cerebral Artery | rno-miR-99a-5p | MIMAT0000820 | 18258830 |
| Middle Cerebral Artery | rno-miR-99b-5p | MIMAT0000821 | 18258830 |
| Middle Cerebral Artery | rno-miR-9a-5p | MIMAT0000781 | 18258830 |
| Milk | mmu-miR-101a-3p | MIMAT0000133 | 19281778 |
| Mitochondria | hsa-miR-27a-3p | MIMAT0000084 | 19513126 |
| Mitochondria | mmu-miR-15b-5p | MIMAT0000124 | 20007690 |
| Mitochondria | mmu-miR-16-5p | MIMAT0000527 | 20007690 |
| Mitochondria | mmu-miR-195a-5p | MIMAT0000225 | 20007690 |
| Mitochondria | mmu-miR-322-5p | MIMAT0000548 | 20007690 |
| Mitochondria | mmu-miR-696 | MIMAT0003483 | 20086200 |
| Mitochondria | rno-miR-15b-5p | MIMAT0000784 | 20007690 |
| Mitochondria | rno-miR-16-5p | MIMAT0000785 | 20007690 |
| Mitochondria | rno-miR-195-5p | MIMAT0000870 | 20007690 |
| Mitochondria | rno-miR-214-3p | MIMAT0000885 | 20007690 |
| Mitochondria | rno-miR-322-5p | MIMAT0001619 | 20007690 |
| Mitochondria | rno-miR-499-5p | MIMAT0003381 | 21186368 |
| Mitochondrial Membranes | rno-miR-1-3p | MIMAT0003125 | 18801338 |
| Monocytes | hsa-miR-146a-5p | MIMAT0000449 | 20375304 |
| Monocytes | hsa-miR-146a-5p | MIMAT0000449 | 21178010 |
| Monocytes | hsa-miR-146a-5p | MIMAT0000449 | 19918258 |
| Monocytes | hsa-miR-146a-5p | MIMAT0000449 | 16885212 |
| Monocytes | hsa-miR-146b-5p | MIMAT0002809 | 16885212 |
| Monocytes | hsa-miR-155-5p | MIMAT0000646 | 21030878 |
| Monocytes | hsa-miR-155-5p | MIMAT0000646 | 19887047 |
| Monocytes | hsa-miR-155-5p | MIMAT0000646 | 19289835 |
| Monocytes | hsa-miR-195-5p | MIMAT0000461 | 20952681 |
| Monocytes | hsa-miR-198 | MIMAT0000228 | 19148268 |
| Monocytes | hsa-miR-214-3p | MIMAT0000271 | 21228352 |
| Monocytes | hsa-miR-663a | MIMAT0003326 | 20622002 |
| Monocytes | hsa-miR-9-5p | MIMAT0000441 | 19289835 |
| Monocytes | mmu-miR-124-3p | MIMAT0000134 | 21131957 |
| Motor Neurons | mmu-miR-128-3p | MIMAT0000140 | 21248104 |
| Motor Neurons | mmu-miR-17-5p | MIMAT0000649 | 21248104 |
| Motor Neurons | mmu-miR-206-3p | MIMAT0000239 | 20007902 |
| Mouth Mucosa | hsa-miR-137 | MIMAT0000429 | 18381414 |
| Mouth Mucosa | hsa-miR-193a-3p | MIMAT0000459 | 18381414 |
| Mouth Mucosa | hsa-miR-221-3p | MIMAT0000278 | 21226887 |
| Mouth Mucosa | hsa-miR-222-3p | MIMAT0000279 | 21226887 |
| Mucous Membrane | hsa-miR-137 | MIMAT0000429 | 20682795 |
| Mucous Membrane | hsa-miR-146a-5p | MIMAT0000449 | 20542134 |
| Mucous Membrane | hsa-miR-155-5p | MIMAT0000646 | 19650740 |
| Mucous Membrane | hsa-miR-195-5p | MIMAT0000461 | 20727858 |
| Mucous Membrane | hsa-miR-196a-5p | MIMAT0000226 | 19342367 |
| Mucous Membrane | hsa-miR-196a-5p | MIMAT0000226 | 19418581 |
| Mucous Membrane | hsa-miR-21-5p | MIMAT0000076 | 19509156 |
| Mucous Membrane | hsa-miR-21-5p | MIMAT0000076 | 21468550 |
| Mucous Membrane | hsa-miR-218-5p | MIMAT0000275 | 19890957 |
| Mucous Membrane | hsa-miR-221-3p | MIMAT0000278 | 21236259 |
| Mucous Membrane | hsa-miR-222-3p | MIMAT0000279 | 22321642 |
| Mucous Membrane | hsa-miR-223-3p | MIMAT0000280 | 22270966 |
| Mucous Membrane | hsa-miR-25-3p | MIMAT0000081 | 23435373 |
| Mucous Membrane | hsa-miR-34b-3p | MIMAT0004676 | 20924086 |
| Mucous Membrane | hsa-miR-34c-5p | MIMAT0000686 | 20924086 |
| Mucous Membrane | hsa-miR-365a-3p | MIMAT0000710 | 22072615 |
| Mucous Membrane | hsa-miR-630 | MIMAT0003299 | 23435373 |
| Mucus | hsa-let-7d-5p | MIMAT0000065 | 21616524 |
| Mucus | hsa-let-7f-5p | MIMAT0000067 | 21616524 |
| Mucus | hsa-let-7g-5p | MIMAT0000414 | 21616524 |
| Mucus | hsa-let-7i-5p | MIMAT0000415 | 21616524 |
| Mucus | hsa-miR-98-5p | MIMAT0000096 | 21616524 |
| Multipotent Stem Cells | mmu-miR-143-3p | MIMAT0000247 | 19578358 |
| Multipotent Stem Cells | mmu-miR-145a-5p | MIMAT0000157 | 19578358 |
| Muscle Cells | hsa-miR-1 | MIMAT0000416 | 19131648 |
| Muscle Cells | hsa-miR-1 | MIMAT0000416 | 20458751 |
| Muscle Cells | hsa-miR-1 | MIMAT0000416 | 21051663 |
| Muscle Cells | hsa-miR-1 | MIMAT0000416 | 19775284 |
| Muscle Cells | hsa-miR-101-3p | MIMAT0000099 | 19818710 |
| Muscle Cells | hsa-miR-125b-5p | MIMAT0000423 | 21200031 |
| Muscle Cells | hsa-miR-133a-3p | MIMAT0000427 | 17344217 |
| Muscle Cells | hsa-miR-133a-3p | MIMAT0000427 | 19720047 |
| Muscle Cells | hsa-miR-133b | MIMAT0000770 | 17344217 |
| Muscle Cells | hsa-miR-133b | MIMAT0000770 | 19720047 |
| Muscle Cells | hsa-miR-150-5p | MIMAT0000451 | 21200031 |
| Muscle Cells | hsa-miR-155-5p | MIMAT0000646 | 17588946 |
| Muscle Cells | hsa-miR-155-5p | MIMAT0000646 | 21310411 |
| Muscle Cells | hsa-miR-195-5p | MIMAT0000461 | 22802111 |
| Muscle Cells | hsa-miR-199a-5p | MIMAT0000231 | 19818710 |
| Muscle Cells | hsa-miR-204-5p | MIMAT0000265 | 22871591 |
| Muscle Cells | hsa-miR-21-5p | MIMAT0000076 | 17478730 |
| Muscle Cells | hsa-miR-21-5p | MIMAT0000076 | 19906824 |
| Muscle Cells | hsa-miR-21-5p | MIMAT0000076 | 20219857 |
| Muscle Cells | hsa-miR-21-5p | MIMAT0000076 | 22034194 |
| Muscle Cells | hsa-miR-21-5p | MIMAT0000076 | 20693317 |
| Muscle Cells | hsa-miR-214-3p | MIMAT0000271 | 19818710 |
| Muscle Cells | hsa-miR-22-3p | MIMAT0000077 | 22538858 |
| Muscle Cells | hsa-miR-221-3p | MIMAT0000278 | 19088079 |
| Muscle Cells | hsa-miR-221-3p | MIMAT0000278 | 19150885 |
| Muscle Cells | hsa-miR-221-3p | MIMAT0000278 | 19859555 |
| Muscle Cells | hsa-miR-222-3p | MIMAT0000279 | 19150885 |
| Muscle Cells | hsa-miR-222-3p | MIMAT0000279 | 19859555 |
| Muscle Cells | hsa-miR-25-3p | MIMAT0000081 | 19541842 |
| Muscle Cells | hsa-miR-26a-5p | MIMAT0000082 | 18281287 |
| Muscle Cells | hsa-miR-26a-5p | MIMAT0000082 | 20857419 |
| Muscle Cells | hsa-miR-26a-5p | MIMAT0000082 | 20525681 |
| Muscle Cells | hsa-miR-27a-3p | MIMAT0000084 | 19666532 |
| Muscle Cells | hsa-miR-27a-3p | MIMAT0000084 | 21149577 |
| Muscle Cells | hsa-miR-27b-3p | MIMAT0000419 | 19666532 |
| Muscle Cells | hsa-miR-31-5p | MIMAT0000089 | 23518389 |
| Muscle Cells | hsa-miR-638 | MIMAT0003308 | 23554459 |
| Muscle Cells | hsa-miR-98-5p | MIMAT0000096 | 19818710 |
| Muscle Cells | mmu-miR-100-3p | MIMAT0017051 | 21339483 |
| Muscle Cells | mmu-miR-100-5p | MIMAT0000655 | 21339483 |
| Muscle Cells | mmu-miR-10a-3p | MIMAT0004659 | 20118242 |
| Muscle Cells | mmu-miR-10a-5p | MIMAT0000648 | 20118242 |
| Muscle Cells | mmu-miR-125b-5p | MIMAT0000136 | 21200031 |
| Muscle Cells | mmu-miR-125b-5p | MIMAT0000136 | 20699419 |
| Muscle Cells | mmu-miR-130a-3p | MIMAT0000141 | 21753805 |
| Muscle Cells | mmu-miR-143-3p | MIMAT0000247 | 20489207 |
| Muscle Cells | mmu-miR-143-3p | MIMAT0000247 | 19578358 |
| Muscle Cells | mmu-miR-143-3p | MIMAT0000247 | 20351064 |
| Muscle Cells | mmu-miR-145a-5p | MIMAT0000157 | 19578358 |
| Muscle Cells | mmu-miR-145a-5p | MIMAT0000157 | 20351064 |
| Muscle Cells | mmu-miR-15b-5p | MIMAT0000124 | 20007690 |
| Muscle Cells | mmu-miR-16-5p | MIMAT0000527 | 20007690 |
| Muscle Cells | mmu-miR-195a-5p | MIMAT0000225 | 20007690 |
| Muscle Cells | mmu-miR-1a-3p | MIMAT0000123 | 19933931 |
| Muscle Cells | mmu-miR-1a-3p | MIMAT0000123 | 20799856 |
| Muscle Cells | mmu-miR-214-3p | MIMAT0000661 | 19818710 |
| Muscle Cells | mmu-miR-21a-5p | MIMAT0000530 | 20404348 |
| Muscle Cells | mmu-miR-26a-5p | MIMAT0000533 | 18281287 |
| Muscle Cells | mmu-miR-322-5p | MIMAT0000548 | 20007690 |
| Muscle Cells | mmu-miR-486-5p | MIMAT0003130 | 20142475 |
| Muscle Cells | mmu-miR-494-3p | MIMAT0003182 | 20837890 |
| Muscle Cells | mmu-miR-696 | MIMAT0003483 | 20086200 |
| Muscle Cells | rno-miR-1-3p | MIMAT0003125 | 19775284 |
| Muscle Cells | rno-miR-133a-3p | MIMAT0000839 | 19720047 |
| Muscle Cells | rno-miR-133b-3p | MIMAT0003126 | 19720047 |
| Muscle Cells | rno-miR-145-5p | MIMAT0000851 | 19542014 |
| Muscle Cells | rno-miR-15b-5p | MIMAT0000784 | 20007690 |
| Muscle Cells | rno-miR-16-5p | MIMAT0000785 | 20007690 |
| Muscle Cells | rno-miR-195-5p | MIMAT0000870 | 20007690 |
| Muscle Cells | rno-miR-208a-3p | MIMAT0000880 | 21720271 |
| Muscle Cells | rno-miR-21-5p | MIMAT0000790 | 17478730 |
| Muscle Cells | rno-miR-21-5p | MIMAT0000790 | 19706597 |
| Muscle Cells | rno-miR-21-5p | MIMAT0000790 | 19336275 |
| Muscle Cells | rno-miR-21-5p | MIMAT0000790 | 19158092 |
| Muscle Cells | rno-miR-214-3p | MIMAT0000885 | 20007690 |
| Muscle Cells | rno-miR-221-3p | MIMAT0000890 | 19150885 |
| Muscle Cells | rno-miR-222-3p | MIMAT0000891 | 19150885 |
| Muscle Cells | rno-miR-27a-3p | MIMAT0000799 | 21149577 |
| Muscle Cells | rno-miR-31a-5p | MIMAT0000810 | 19542014 |
| Muscle Cells | rno-miR-31a-5p | MIMAT0000810 | 22020941 |
| Muscle Cells | rno-miR-322-5p | MIMAT0001619 | 20007690 |
| Muscle Fibers Skeletal | hsa-miR-133b | MIMAT0000770 | 17210790 |
| Muscle Fibers Skeletal | hsa-miR-221-3p | MIMAT0000278 | 19859555 |
| Muscle Fibers Skeletal | hsa-miR-222-3p | MIMAT0000279 | 19859555 |
| Muscle Fibers Skeletal | hsa-miR-26a-5p | MIMAT0000082 | 18281287 |
| Muscle Fibers Skeletal | mmu-miR-133b-3p | MIMAT0000769 | 17210790 |
| Muscle Fibers Skeletal | mmu-miR-206-3p | MIMAT0000239 | 21041476 |
| Muscle Fibers Skeletal | mmu-miR-206-3p | MIMAT0000239 | 20007902 |
| Muscle Fibers Skeletal | mmu-miR-26a-5p | MIMAT0000533 | 18281287 |
| Muscle Fibers Skeletal | mmu-miR-486-5p | MIMAT0003130 | 21041476 |
| Muscle Skeletal | bta-miR-29a | MIMAT0003518 | 17652184 |
| Muscle Skeletal | bta-miR-29c | MIMAT0003829 | 17652184 |
| Muscle Skeletal | cfa-miR-29a | MIMAT0006626 | 17652184 |
| Muscle Skeletal | cfa-miR-29c | MIMAT0006705 | 17652184 |
| Muscle Skeletal | dre-miR-29a | MIMAT0001802 | 17652184 |
| Muscle Skeletal | hsa-miR-1 | MIMAT0000416 | 15951802 |
| Muscle Skeletal | hsa-miR-1 | MIMAT0000416 | 21169019 |
| Muscle Skeletal | hsa-miR-1 | MIMAT0000416 | 20956382 |
| Muscle Skeletal | hsa-miR-1 | MIMAT0000416 | 19710019 |
| Muscle Skeletal | hsa-miR-1 | MIMAT0000416 | 20458751 |
| Muscle Skeletal | hsa-miR-101-3p | MIMAT0000099 | 19818710 |
| Muscle Skeletal | hsa-miR-199a-5p | MIMAT0000231 | 19818710 |
| Muscle Skeletal | hsa-miR-206 | MIMAT0000462 | 20956382 |
| Muscle Skeletal | hsa-miR-206 | MIMAT0000462 | 19710019 |
| Muscle Skeletal | hsa-miR-206 | MIMAT0000462 | 19620785 |
| Muscle Skeletal | hsa-miR-206 | MIMAT0000462 | 17030984 |
| Muscle Skeletal | hsa-miR-214-3p | MIMAT0000271 | 19818710 |
| Muscle Skeletal | hsa-miR-221-3p | MIMAT0000278 | 19859555 |
| Muscle Skeletal | hsa-miR-222-3p | MIMAT0000279 | 19859555 |
| Muscle Skeletal | hsa-miR-26a-5p | MIMAT0000082 | 18281287 |
| Muscle Skeletal | hsa-miR-27a-3p | MIMAT0000084 | 19666532 |
| Muscle Skeletal | hsa-miR-27b-3p | MIMAT0000419 | 19666532 |
| Muscle Skeletal | hsa-miR-29a-3p | MIMAT0000086 | 21169019 |
| Muscle Skeletal | hsa-miR-98-5p | MIMAT0000096 | 19818710 |
| Muscle Skeletal | mmu-miR-133a-3p | MIMAT0000145 | 16380711 |
| Muscle Skeletal | mmu-miR-133a-3p | MIMAT0000145 | 19073597 |
| Muscle Skeletal | mmu-miR-1a-3p | MIMAT0000123 | 19933931 |
| Muscle Skeletal | mmu-miR-1a-3p | MIMAT0000123 | 20799856 |
| Muscle Skeletal | mmu-miR-1a-3p | MIMAT0000123 | 15951802 |
| Muscle Skeletal | mmu-miR-1a-3p | MIMAT0000123 | 20819939 |
| Muscle Skeletal | mmu-miR-1a-3p | MIMAT0000123 | 16380711 |
| Muscle Skeletal | mmu-miR-1a-3p | MIMAT0000123 | 19073597 |
| Muscle Skeletal | mmu-miR-206-3p | MIMAT0000239 | 17030984 |
| Muscle Skeletal | mmu-miR-206-3p | MIMAT0000239 | 20007902 |
| Muscle Skeletal | mmu-miR-206-3p | MIMAT0000239 | 20819939 |
| Muscle Skeletal | mmu-miR-210-3p | MIMAT0000658 | 19073597 |
| Muscle Skeletal | mmu-miR-214-3p | MIMAT0000661 | 19818710 |
| Muscle Skeletal | mmu-miR-26a-5p | MIMAT0000533 | 18281287 |
| Muscle Skeletal | mmu-miR-29a-3p | MIMAT0000535 | 17652184 |
| Muscle Skeletal | mmu-miR-29b-3p | MIMAT0000127 | 17652184 |
| Muscle Skeletal | mmu-miR-29c-3p | MIMAT0000536 | 17652184 |
| Muscle Skeletal | mmu-miR-696 | MIMAT0003483 | 20086200 |
| Muscle Skeletal | ppy-miR-29a | MIMAT0002363 | 17652184 |
| Muscle Skeletal | ppy-miR-29c | MIMAT0015735 | 17652184 |
| Muscle Skeletal | ptr-miR-29a | MIMAT0002364 | 17652184 |
| Muscle Skeletal | ptr-miR-29c | MIMAT0008082 | 17652184 |
| Muscle Skeletal | rno-miR-24-3p | MIMAT0000794 | 19499150 |
| Muscle Skeletal | rno-miR-29a-3p | MIMAT0000802 | 17652184 |
| Muscle Skeletal | rno-miR-29b-3p | MIMAT0000801 | 17652184 |
| Muscle Skeletal | rno-miR-29c-3p | MIMAT0000803 | 17652184 |
| Muscle Skeletal | ssc-miR-29a | MIMAT0013870 | 17652184 |
| Muscle Skeletal | ssc-miR-29c | MIMAT0002166 | 17652184 |
| Muscle Skeletal | xtr-miR-29a | MIMAT0003656 | 17652184 |
| Muscle Smooth | hsa-miR-1 | MIMAT0000416 | 20458751 |
| Muscle Smooth | hsa-miR-1 | MIMAT0000416 | 21051663 |
| Muscle Smooth | hsa-miR-155-5p | MIMAT0000646 | 17588946 |
| Muscle Smooth | hsa-miR-155-5p | MIMAT0000646 | 21310411 |
| Muscle Smooth | hsa-miR-195-5p | MIMAT0000461 | 22802111 |
| Muscle Smooth | hsa-miR-204-5p | MIMAT0000265 | 22871591 |
| Muscle Smooth | hsa-miR-21-5p | MIMAT0000076 | 20693317 |
| Muscle Smooth | hsa-miR-21-5p | MIMAT0000076 | 17478730 |
| Muscle Smooth | hsa-miR-21-5p | MIMAT0000076 | 19906824 |
| Muscle Smooth | hsa-miR-21-5p | MIMAT0000076 | 22034194 |
| Muscle Smooth | hsa-miR-22-3p | MIMAT0000077 | 22538858 |
| Muscle Smooth | hsa-miR-221-3p | MIMAT0000278 | 19088079 |
| Muscle Smooth | hsa-miR-221-3p | MIMAT0000278 | 19150885 |
| Muscle Smooth | hsa-miR-222-3p | MIMAT0000279 | 19150885 |
| Muscle Smooth | hsa-miR-25-3p | MIMAT0000081 | 19541842 |
| Muscle Smooth | hsa-miR-26a-5p | MIMAT0000082 | 20857419 |
| Muscle Smooth | hsa-miR-26a-5p | MIMAT0000082 | 20525681 |
| Muscle Smooth | hsa-miR-31-5p | MIMAT0000089 | 23518389 |
| Muscle Smooth | hsa-miR-638 | MIMAT0003308 | 23554459 |
| Muscle Smooth | mmu-miR-100-3p | MIMAT0017051 | 21339483 |
| Muscle Smooth | mmu-miR-100-5p | MIMAT0000655 | 21339483 |
| Muscle Smooth | mmu-miR-10a-3p | MIMAT0004659 | 20118242 |
| Muscle Smooth | mmu-miR-10a-5p | MIMAT0000648 | 20118242 |
| Muscle Smooth | mmu-miR-125b-5p | MIMAT0000136 | 20699419 |
| Muscle Smooth | mmu-miR-130a-3p | MIMAT0000141 | 21753805 |
| Muscle Smooth | mmu-miR-143-3p | MIMAT0000247 | 20351064 |
| Muscle Smooth | mmu-miR-143-3p | MIMAT0000247 | 20489207 |
| Muscle Smooth | mmu-miR-143-3p | MIMAT0000247 | 19578358 |
| Muscle Smooth | mmu-miR-145a-5p | MIMAT0000157 | 19578358 |
| Muscle Smooth | mmu-miR-145a-5p | MIMAT0000157 | 20351064 |
| Muscle Smooth | mmu-miR-1a-3p | MIMAT0000123 | 20799856 |
| Muscle Smooth | rno-miR-145-5p | MIMAT0000851 | 19542014 |
| Muscle Smooth | rno-miR-150-5p | MIMAT0000853 | 19892940 |
| Muscle Smooth | rno-miR-194-5p | MIMAT0000869 | 19892940 |
| Muscle Smooth | rno-miR-208a-3p | MIMAT0000880 | 21720271 |
| Muscle Smooth | rno-miR-21-5p | MIMAT0000790 | 17478730 |
| Muscle Smooth | rno-miR-21-5p | MIMAT0000790 | 19158092 |
| Muscle Smooth | rno-miR-221-3p | MIMAT0000890 | 19150885 |
| Muscle Smooth | rno-miR-222-3p | MIMAT0000891 | 19150885 |
| Muscle Smooth | rno-miR-31a-5p | MIMAT0000810 | 19542014 |
| Muscle Smooth | rno-miR-31a-5p | MIMAT0000810 | 22020941 |
| Muscle Smooth Vascular | hsa-miR-155-5p | MIMAT0000646 | 17588946 |
| Muscle Smooth Vascular | hsa-miR-195-5p | MIMAT0000461 | 22802111 |
| Muscle Smooth Vascular | hsa-miR-204-5p | MIMAT0000265 | 22871591 |
| Muscle Smooth Vascular | hsa-miR-21-5p | MIMAT0000076 | 22034194 |
| Muscle Smooth Vascular | hsa-miR-21-5p | MIMAT0000076 | 20693317 |
| Muscle Smooth Vascular | hsa-miR-221-3p | MIMAT0000278 | 19088079 |
| Muscle Smooth Vascular | hsa-miR-221-3p | MIMAT0000278 | 19150885 |
| Muscle Smooth Vascular | hsa-miR-222-3p | MIMAT0000279 | 19150885 |
| Muscle Smooth Vascular | hsa-miR-31-5p | MIMAT0000089 | 23518389 |
| Muscle Smooth Vascular | hsa-miR-638 | MIMAT0003308 | 23554459 |
| Muscle Smooth Vascular | mmu-miR-100-3p | MIMAT0017051 | 21339483 |
| Muscle Smooth Vascular | mmu-miR-100-5p | MIMAT0000655 | 21339483 |
| Muscle Smooth Vascular | mmu-miR-125b-5p | MIMAT0000136 | 20699419 |
| Muscle Smooth Vascular | mmu-miR-130a-3p | MIMAT0000141 | 21753805 |
| Muscle Smooth Vascular | mmu-miR-143-3p | MIMAT0000247 | 19578358 |
| Muscle Smooth Vascular | mmu-miR-145a-5p | MIMAT0000157 | 19578358 |
| Muscle Smooth Vascular | rno-miR-145-5p | MIMAT0000851 | 19542014 |
| Muscle Smooth Vascular | rno-miR-208a-3p | MIMAT0000880 | 21720271 |
| Muscle Smooth Vascular | rno-miR-21-5p | MIMAT0000790 | 19158092 |
| Muscle Smooth Vascular | rno-miR-221-3p | MIMAT0000890 | 19150885 |
| Muscle Smooth Vascular | rno-miR-222-3p | MIMAT0000891 | 19150885 |
| Muscle Smooth Vascular | rno-miR-31a-5p | MIMAT0000810 | 19542014 |
| Muscle Smooth Vascular | rno-miR-31a-5p | MIMAT0000810 | 22020941 |
| Muscles | bta-miR-29a | MIMAT0003518 | 17652184 |
| Muscles | bta-miR-29c | MIMAT0003829 | 17652184 |
| Muscles | cfa-miR-29a | MIMAT0006626 | 17652184 |
| Muscles | cfa-miR-29c | MIMAT0006705 | 17652184 |
| Muscles | dre-miR-29a | MIMAT0001802 | 17652184 |
| Muscles | hsa-miR-1 | MIMAT0000416 | 21169019 |
| Muscles | hsa-miR-1 | MIMAT0000416 | 19506341 |
| Muscles | hsa-miR-1 | MIMAT0000416 | 19710019 |
| Muscles | hsa-miR-125b-5p | MIMAT0000423 | 21200031 |
| Muscles | hsa-miR-146a-5p | MIMAT0000449 | 21968601 |
| Muscles | hsa-miR-150-5p | MIMAT0000451 | 21200031 |
| Muscles | hsa-miR-195-5p | MIMAT0000461 | 22802111 |
| Muscles | hsa-miR-204-5p | MIMAT0000265 | 22871591 |
| Muscles | hsa-miR-206 | MIMAT0000462 | 19710019 |
| Muscles | hsa-miR-21-5p | MIMAT0000076 | 22034194 |
| Muscles | hsa-miR-22-3p | MIMAT0000077 | 22538858 |
| Muscles | hsa-miR-222-3p | MIMAT0000279 | 23447020 |
| Muscles | hsa-miR-26a-5p | MIMAT0000082 | 20857419 |
| Muscles | hsa-miR-29a-3p | MIMAT0000086 | 21169019 |
| Muscles | hsa-miR-31-5p | MIMAT0000089 | 23518389 |
| Muscles | hsa-miR-378a-3p | MIMAT0000732 | 21471220 |
| Muscles | hsa-miR-503-5p | MIMAT0002874 | 21220732 |
| Muscles | hsa-miR-638 | MIMAT0003308 | 23554459 |
| Muscles | mmu-miR-100-3p | MIMAT0017051 | 21339483 |
| Muscles | mmu-miR-100-5p | MIMAT0000655 | 21339483 |
| Muscles | mmu-miR-125b-5p | MIMAT0000136 | 21200031 |
| Muscles | mmu-miR-128-3p | MIMAT0000140 | 21248104 |
| Muscles | mmu-miR-130a-3p | MIMAT0000141 | 21753805 |
| Muscles | mmu-miR-17-5p | MIMAT0000649 | 21248104 |
| Muscles | mmu-miR-206-3p | MIMAT0000239 | 20007902 |
| Muscles | mmu-miR-29a-3p | MIMAT0000535 | 17652184 |
| Muscles | mmu-miR-29b-3p | MIMAT0000127 | 17652184 |
| Muscles | mmu-miR-29c-3p | MIMAT0000536 | 17652184 |
| Muscles | ppy-miR-29a | MIMAT0002363 | 17652184 |
| Muscles | ppy-miR-29c | MIMAT0015735 | 17652184 |
| Muscles | ptr-miR-29a | MIMAT0002364 | 17652184 |
| Muscles | ptr-miR-29c | MIMAT0008082 | 17652184 |
| Muscles | rno-miR-1-3p | MIMAT0003125 | 19506341 |
| Muscles | rno-miR-146a-5p | MIMAT0000852 | 22735812 |
| Muscles | rno-miR-208a-3p | MIMAT0000880 | 21720271 |
| Muscles | rno-miR-21-5p | MIMAT0000790 | 20553627 |
| Muscles | rno-miR-29a-3p | MIMAT0000802 | 17652184 |
| Muscles | rno-miR-29b-3p | MIMAT0000801 | 17652184 |
| Muscles | rno-miR-29c-3p | MIMAT0000803 | 17652184 |
| Muscles | rno-miR-31a-5p | MIMAT0000810 | 22020941 |
| Muscles | ssc-miR-29a | MIMAT0013870 | 17652184 |
| Muscles | ssc-miR-29c | MIMAT0002166 | 17652184 |
| Muscles | xtr-miR-29a | MIMAT0003656 | 17652184 |
| Myelin Sheath | mmu-miR-219a-5p | MIMAT0000664 | 20223198 |
| Myelin Sheath | mmu-miR-23a-3p | MIMAT0000532 | 19259393 |
| Myelin Sheath | mmu-miR-23b-3p | MIMAT0000125 | 19259393 |
| Myelin Sheath | rno-miR-29a-3p | MIMAT0000802 | 19170179 |
| Myelin Sheath | rno-miR-9a-5p | MIMAT0000781 | 18987208 |
| Myeloid Cells | hsa-miR-155-5p | MIMAT0000646 | 21036908 |
| Myeloid Cells | hsa-miR-155-5p | MIMAT0000646 | 18299402 |
| Myeloid Cells | hsa-miR-223-3p | MIMAT0000280 | 20029046 |
| Myeloid Cells | hsa-miR-92a-3p | MIMAT0000092 | 19608627 |
| Myeloid Cells | mmu-miR-92a-3p | MIMAT0000539 | 19608627 |
| Myoblasts | hsa-miR-1 | MIMAT0000416 | 20956382 |
| Myoblasts | hsa-miR-125b-5p | MIMAT0000423 | 21200031 |
| Myoblasts | hsa-miR-133b | MIMAT0000770 | 17210790 |
| Myoblasts | hsa-miR-150-5p | MIMAT0000451 | 21200031 |
| Myoblasts | hsa-miR-181a-5p | MIMAT0000256 | 16489342 |
| Myoblasts | hsa-miR-206 | MIMAT0000462 | 20956382 |
| Myoblasts | hsa-miR-221-3p | MIMAT0000278 | 19859555 |
| Myoblasts | hsa-miR-222-3p | MIMAT0000279 | 19859555 |
| Myoblasts | hsa-miR-26a-5p | MIMAT0000082 | 18281287 |
| Myoblasts | hsa-miR-31-5p | MIMAT0000089 | 21212803 |
| Myoblasts | hsa-miR-378a-3p | MIMAT0000732 | 21471220 |
| Myoblasts | mmu-miR-125b-5p | MIMAT0000136 | 21200031 |
| Myoblasts | mmu-miR-133a-3p | MIMAT0000145 | 16380711 |
| Myoblasts | mmu-miR-133a-3p | MIMAT0000145 | 16923828 |
| Myoblasts | mmu-miR-133b-3p | MIMAT0000769 | 17210790 |
| Myoblasts | mmu-miR-137-3p | MIMAT0000149 | 20534588 |
| Myoblasts | mmu-miR-137-5p | MIMAT0016986 | 20534588 |
| Myoblasts | mmu-miR-181a-5p | MIMAT0000210 | 16489342 |
| Myoblasts | mmu-miR-1a-3p | MIMAT0000123 | 20819939 |
| Myoblasts | mmu-miR-1a-3p | MIMAT0000123 | 16380711 |
| Myoblasts | mmu-miR-1a-3p | MIMAT0000123 | 16923828 |
| Myoblasts | mmu-miR-206-3p | MIMAT0000239 | 21041476 |
| Myoblasts | mmu-miR-206-3p | MIMAT0000239 | 20819939 |
| Myoblasts | mmu-miR-206-3p | MIMAT0000239 | 16923828 |
| Myoblasts | mmu-miR-26a-5p | MIMAT0000533 | 18281287 |
| Myoblasts | mmu-miR-486-5p | MIMAT0003130 | 21041476 |
| Myocardium | hsa-miR-1 | MIMAT0000416 | 20081117 |
| Myocardium | hsa-miR-1 | MIMAT0000416 | 19506341 |
| Myocardium | hsa-miR-133a-3p | MIMAT0000427 | 19720047 |
| Myocardium | hsa-miR-133b | MIMAT0000770 | 19720047 |
| Myocardium | hsa-miR-155-5p | MIMAT0000646 | 20550618 |
| Myocardium | hsa-miR-499a-5p | MIMAT0002870 | 20081117 |
| Myocardium | hsa-miR-499b-5p | MIMAT0019897 | 20081117 |
| Myocardium | rno-miR-1-3p | MIMAT0003125 | 19506341 |
| Myocardium | rno-miR-133a-3p | MIMAT0000839 | 19720047 |
| Myocardium | rno-miR-133b-3p | MIMAT0003126 | 19720047 |
| Myocardium | rno-miR-31a-5p | MIMAT0000810 | 23270756 |
| Myocardium | rno-miR-384-5p | MIMAT0005309 | 23315007 |
| Myocytes Cardiac | hsa-miR-1 | MIMAT0000416 | 21051663 |
| Myocytes Cardiac | hsa-miR-1 | MIMAT0000416 | 15951802 |
| Myocytes Cardiac | hsa-miR-1 | MIMAT0000416 | 19188439 |
| Myocytes Cardiac | hsa-miR-1 | MIMAT0000416 | 23141496 |
| Myocytes Cardiac | hsa-miR-1 | MIMAT0000416 | 20571053 |
| Myocytes Cardiac | hsa-miR-1 | MIMAT0000416 | 20081117 |
| Myocytes Cardiac | hsa-miR-1 | MIMAT0000416 | 19506341 |
| Myocytes Cardiac | hsa-miR-126-3p | MIMAT0000445 | 20571053 |
| Myocytes Cardiac | hsa-miR-133a-3p | MIMAT0000427 | 19720047 |
| Myocytes Cardiac | hsa-miR-133a-3p | MIMAT0000427 | 23069713 |
| Myocytes Cardiac | hsa-miR-133b | MIMAT0000770 | 19720047 |
| Myocytes Cardiac | hsa-miR-150-5p | MIMAT0000451 | 23211718 |
| Myocytes Cardiac | hsa-miR-208a-3p | MIMAT0000241 | 17379774 |
| Myocytes Cardiac | hsa-miR-21-5p | MIMAT0000076 | 20219857 |
| Myocytes Cardiac | hsa-miR-223-3p | MIMAT0000280 | 20080987 |
| Myocytes Cardiac | hsa-miR-27a-3p | MIMAT0000084 | 21149577 |
| Myocytes Cardiac | hsa-miR-320a | MIMAT0000510 | 19380620 |
| Myocytes Cardiac | hsa-miR-499a-5p | MIMAT0002870 | 20081117 |
| Myocytes Cardiac | hsa-miR-499b-5p | MIMAT0019897 | 20081117 |
| Myocytes Cardiac | hsa-miR-9-5p | MIMAT0000441 | 19188439 |
| Myocytes Cardiac | mmu-miR-130a-3p | MIMAT0000141 | 19582148 |
| Myocytes Cardiac | mmu-miR-133a-3p | MIMAT0000145 | 17468766 |
| Myocytes Cardiac | mmu-miR-133a-3p | MIMAT0000145 | 20173049 |
| Myocytes Cardiac | mmu-miR-133a-3p | MIMAT0000145 | 19043405 |
| Myocytes Cardiac | mmu-miR-138-5p | MIMAT0000150 | 19004786 |
| Myocytes Cardiac | mmu-miR-15b-5p | MIMAT0000124 | 20007690 |
| Myocytes Cardiac | mmu-miR-16-5p | MIMAT0000527 | 20007690 |
| Myocytes Cardiac | mmu-miR-195a-5p | MIMAT0000225 | 20007690 |
| Myocytes Cardiac | mmu-miR-195a-5p | MIMAT0000225 | 21622680 |
| Myocytes Cardiac | mmu-miR-1a-3p | MIMAT0000123 | 19188439 |
| Myocytes Cardiac | mmu-miR-1a-3p | MIMAT0000123 | 15951802 |
| Myocytes Cardiac | mmu-miR-1a-3p | MIMAT0000123 | 23436819 |
| Myocytes Cardiac | mmu-miR-206-3p | MIMAT0000239 | 19043405 |
| Myocytes Cardiac | mmu-miR-208a-3p | MIMAT0000520 | 17379774 |
| Myocytes Cardiac | mmu-miR-21a-5p | MIMAT0000530 | 20404348 |
| Myocytes Cardiac | mmu-miR-21a-5p | MIMAT0000530 | 19043405 |
| Myocytes Cardiac | mmu-miR-320-3p | MIMAT0000666 | 19380620 |
| Myocytes Cardiac | mmu-miR-322-5p | MIMAT0000548 | 20007690 |
| Myocytes Cardiac | mmu-miR-494-3p | MIMAT0003182 | 20837890 |
| Myocytes Cardiac | rno-miR-1-3p | MIMAT0003125 | 23436819 |
| Myocytes Cardiac | rno-miR-1-3p | MIMAT0003125 | 19188439 |
| Myocytes Cardiac | rno-miR-1-3p | MIMAT0003125 | 19506341 |
| Myocytes Cardiac | rno-miR-1-3p | MIMAT0003125 | 18801338 |
| Myocytes Cardiac | rno-miR-133a-3p | MIMAT0000839 | 19720047 |
| Myocytes Cardiac | rno-miR-133b-3p | MIMAT0003126 | 19720047 |
| Myocytes Cardiac | rno-miR-15b-5p | MIMAT0000784 | 20007690 |
| Myocytes Cardiac | rno-miR-16-5p | MIMAT0000785 | 20007690 |
| Myocytes Cardiac | rno-miR-195-5p | MIMAT0000870 | 20007690 |
| Myocytes Cardiac | rno-miR-21-5p | MIMAT0000790 | 19706597 |
| Myocytes Cardiac | rno-miR-21-5p | MIMAT0000790 | 19336275 |
| Myocytes Cardiac | rno-miR-214-3p | MIMAT0000885 | 20007690 |
| Myocytes Cardiac | rno-miR-27a-3p | MIMAT0000799 | 21149577 |
| Myocytes Cardiac | rno-miR-322-5p | MIMAT0001619 | 20007690 |
| Myocytes Cardiac | rno-miR-499-5p | MIMAT0003381 | 21186368 |
| Myocytes Smooth Muscle | hsa-miR-1 | MIMAT0000416 | 21051663 |
| Myocytes Smooth Muscle | hsa-miR-1 | MIMAT0000416 | 20458751 |
| Myocytes Smooth Muscle | hsa-miR-155-5p | MIMAT0000646 | 21310411 |
| Myocytes Smooth Muscle | hsa-miR-155-5p | MIMAT0000646 | 17588946 |
| Myocytes Smooth Muscle | hsa-miR-195-5p | MIMAT0000461 | 22802111 |
| Myocytes Smooth Muscle | hsa-miR-204-5p | MIMAT0000265 | 22871591 |
| Myocytes Smooth Muscle | hsa-miR-21-5p | MIMAT0000076 | 22034194 |
| Myocytes Smooth Muscle | hsa-miR-21-5p | MIMAT0000076 | 17478730 |
| Myocytes Smooth Muscle | hsa-miR-21-5p | MIMAT0000076 | 20693317 |
| Myocytes Smooth Muscle | hsa-miR-21-5p | MIMAT0000076 | 19906824 |
| Myocytes Smooth Muscle | hsa-miR-22-3p | MIMAT0000077 | 22538858 |
| Myocytes Smooth Muscle | hsa-miR-221-3p | MIMAT0000278 | 19088079 |
| Myocytes Smooth Muscle | hsa-miR-221-3p | MIMAT0000278 | 19150885 |
| Myocytes Smooth Muscle | hsa-miR-222-3p | MIMAT0000279 | 19150885 |
| Myocytes Smooth Muscle | hsa-miR-25-3p | MIMAT0000081 | 19541842 |
| Myocytes Smooth Muscle | hsa-miR-26a-5p | MIMAT0000082 | 20857419 |
| Myocytes Smooth Muscle | hsa-miR-26a-5p | MIMAT0000082 | 20525681 |
| Myocytes Smooth Muscle | hsa-miR-31-5p | MIMAT0000089 | 23518389 |
| Myocytes Smooth Muscle | hsa-miR-638 | MIMAT0003308 | 23554459 |
| Myocytes Smooth Muscle | mmu-miR-100-3p | MIMAT0017051 | 21339483 |
| Myocytes Smooth Muscle | mmu-miR-100-5p | MIMAT0000655 | 21339483 |
| Myocytes Smooth Muscle | mmu-miR-10a-3p | MIMAT0004659 | 20118242 |
| Myocytes Smooth Muscle | mmu-miR-10a-5p | MIMAT0000648 | 20118242 |
| Myocytes Smooth Muscle | mmu-miR-125b-5p | MIMAT0000136 | 20699419 |
| Myocytes Smooth Muscle | mmu-miR-130a-3p | MIMAT0000141 | 21753805 |
| Myocytes Smooth Muscle | mmu-miR-143-3p | MIMAT0000247 | 20351064 |
| Myocytes Smooth Muscle | mmu-miR-143-3p | MIMAT0000247 | 19578358 |
| Myocytes Smooth Muscle | mmu-miR-143-3p | MIMAT0000247 | 20489207 |
| Myocytes Smooth Muscle | mmu-miR-145a-5p | MIMAT0000157 | 20351064 |
| Myocytes Smooth Muscle | mmu-miR-145a-5p | MIMAT0000157 | 19578358 |
| Myocytes Smooth Muscle | mmu-miR-1a-3p | MIMAT0000123 | 20799856 |
| Myocytes Smooth Muscle | rno-miR-145-5p | MIMAT0000851 | 19542014 |
| Myocytes Smooth Muscle | rno-miR-208a-3p | MIMAT0000880 | 21720271 |
| Myocytes Smooth Muscle | rno-miR-21-5p | MIMAT0000790 | 19158092 |
| Myocytes Smooth Muscle | rno-miR-21-5p | MIMAT0000790 | 17478730 |
| Myocytes Smooth Muscle | rno-miR-221-3p | MIMAT0000890 | 19150885 |
| Myocytes Smooth Muscle | rno-miR-222-3p | MIMAT0000891 | 19150885 |
| Myocytes Smooth Muscle | rno-miR-31a-5p | MIMAT0000810 | 19542014 |
| Myocytes Smooth Muscle | rno-miR-31a-5p | MIMAT0000810 | 22020941 |
| Myofibroblasts | hsa-miR-146a-5p | MIMAT0000449 | 21968601 |
| Myofibroblasts | mmu-miR-132-3p | MIMAT0000144 | 19843474 |
| Myofibroblasts | rno-miR-132-3p | MIMAT0000838 | 19843474 |
| Myofibroblasts | rno-miR-21-5p | MIMAT0000790 | 22565856 |
| Myofibroblasts | rno-miR-31a-5p | MIMAT0000810 | 23270756 |
| Myometrium | hsa-let-7c-5p | MIMAT0000064 | 17243163 |
| Nasopharynx | hsa-miR-200a-3p | MIMAT0000682 | 19931509 |
| Neck | hsa-miR-101-3p | MIMAT0000099 | 19043531 |
| Neck | hsa-miR-125a-5p | MIMAT0000443 | 19179615 |
| Neck | hsa-miR-135b-5p | MIMAT0000758 | 23340180 |
| Neck | hsa-miR-138-5p | MIMAT0000430 | 21770894 |
| Neck | hsa-miR-15a-5p | MIMAT0000068 | 19117988 |
| Neck | hsa-miR-181a-5p | MIMAT0000256 | 21274007 |
| Neck | hsa-miR-188-5p | MIMAT0000457 | 19223510 |
| Neck | hsa-miR-200c-3p | MIMAT0000617 | 21294122 |
| Neck | hsa-miR-200c-3p | MIMAT0000617 | 19223510 |
| Neck | hsa-miR-200c-3p | MIMAT0000617 | 21207483 |
| Neck | hsa-miR-203a | MIMAT0000264 | 18483491 |
| Neck | hsa-miR-204-5p | MIMAT0000265 | 20369013 |
| Neck | hsa-miR-21-5p | MIMAT0000076 | 18372920 |
| Neck | hsa-miR-30c-5p | MIMAT0000244 | 19223510 |
| Neck | hsa-miR-30e-5p | MIMAT0000692 | 19223510 |
| Neck | hsa-miR-31-3p | MIMAT0004504 | 20145132 |
| Neck | hsa-miR-31-5p | MIMAT0000089 | 20145132 |
| Neck | hsa-miR-34a-5p | MIMAT0000255 | 21294122 |
| Neck | hsa-miR-34a-5p | MIMAT0000255 | 21240262 |
| Neck | hsa-miR-374a-5p | MIMAT0000727 | 21274007 |
| Neck | hsa-miR-489-3p | MIMAT0002805 | 20700123 |
| Neck | hsa-miR-519a-3p | MIMAT0002869 | 21274007 |
| Neck | hsa-miR-630 | MIMAT0003299 | 21274007 |
| Neck | hsa-miR-98-5p | MIMAT0000096 | 17222355 |
| Neck | mmu-miR-204-5p | MIMAT0000237 | 20369013 |
| Neck | mmu-miR-21a-5p | MIMAT0000530 | 18372920 |
| Neoplastic Stem Cells | hsa-let-7a-5p | MIMAT0000062 | 19878981 |
| Neoplastic Stem Cells | hsa-miR-124-3p | MIMAT0000422 | 18577219 |
| Neoplastic Stem Cells | hsa-miR-128-3p | MIMAT0000424 | 20574517 |
| Neoplastic Stem Cells | hsa-miR-137 | MIMAT0000429 | 18577219 |
| Neoplastic Stem Cells | hsa-miR-145-5p | MIMAT0000437 | 21496429 |
| Neoplastic Stem Cells | hsa-miR-145-5p | MIMAT0000437 | 20382729 |
| Neoplastic Stem Cells | hsa-miR-145-5p | MIMAT0000437 | 20668064 |
| Neoplastic Stem Cells | hsa-miR-153-3p | MIMAT0000439 | 20668064 |
| Neoplastic Stem Cells | hsa-miR-16-5p | MIMAT0000069 | 20668064 |
| Neoplastic Stem Cells | hsa-miR-181a-5p | MIMAT0000256 | 19585654 |
| Neoplastic Stem Cells | hsa-miR-181b-5p | MIMAT0000257 | 19585654 |
| Neoplastic Stem Cells | hsa-miR-181c-5p | MIMAT0000258 | 19585654 |
| Neoplastic Stem Cells | hsa-miR-181d-5p | MIMAT0002821 | 19585654 |
| Neoplastic Stem Cells | hsa-miR-199b-5p | MIMAT0000263 | 23354452 |
| Neoplastic Stem Cells | hsa-miR-199b-5p | MIMAT0000263 | 19308264 |
| Neoplastic Stem Cells | hsa-miR-200a-3p | MIMAT0000682 | 21529905 |
| Neoplastic Stem Cells | hsa-miR-200c-3p | MIMAT0000617 | 19665978 |
| Neoplastic Stem Cells | hsa-miR-200c-3p | MIMAT0000617 | 21294122 |
| Neoplastic Stem Cells | hsa-miR-200c-3p | MIMAT0000617 | 21207483 |
| Neoplastic Stem Cells | hsa-miR-203a | MIMAT0000264 | 20668064 |
| Neoplastic Stem Cells | hsa-miR-21-5p | MIMAT0000076 | 22072622 |
| Neoplastic Stem Cells | hsa-miR-21-5p | MIMAT0000076 | 22528454 |
| Neoplastic Stem Cells | hsa-miR-217 | MIMAT0000274 | 20668064 |
| Neoplastic Stem Cells | hsa-miR-302a-3p | MIMAT0000684 | 21062975 |
| Neoplastic Stem Cells | hsa-miR-302b-3p | MIMAT0000715 | 21062975 |
| Neoplastic Stem Cells | hsa-miR-302c-3p | MIMAT0000717 | 21062975 |
| Neoplastic Stem Cells | hsa-miR-302d-3p | MIMAT0000718 | 21062975 |
| Neoplastic Stem Cells | hsa-miR-31-5p | MIMAT0000089 | 21048943 |
| Neoplastic Stem Cells | hsa-miR-34a-5p | MIMAT0000255 | 21240262 |
| Neoplastic Stem Cells | hsa-miR-34a-5p | MIMAT0000255 | 21294122 |
| Neoplastic Stem Cells | hsa-miR-34a-5p | MIMAT0000255 | 18803879 |
| Neoplastic Stem Cells | hsa-miR-34b-3p | MIMAT0004676 | 18803879 |
| Neoplastic Stem Cells | hsa-miR-34b-5p | MIMAT0000685 | 18803879 |
| Neoplastic Stem Cells | hsa-miR-34c-5p | MIMAT0000686 | 18803879 |
| Neoplastic Stem Cells | mmu-miR-124-3p | MIMAT0000134 | 18577219 |
| Neoplastic Stem Cells | mmu-miR-137-3p | MIMAT0000149 | 18577219 |
| Neoplastic Stem Cells | mmu-miR-137-5p | MIMAT0016986 | 18577219 |
| Nerve Fibers | hsa-miR-7-5p | MIMAT0000252 | 21156648 |
| Nerve Tissue | hsa-miR-7-5p | MIMAT0000252 | 21156648 |
| Nervous System | bta-miR-132 | MIMAT0003812 | 17553428 |
| Nervous System | bta-miR-219 | MIMAT0030444 | 17553428 |
| Nervous System | cfa-miR-132 | MIMAT0006732 | 17553428 |
| Nervous System | dre-miR-219 | MIMAT0001286 | 17553428 |
| Nervous System | hsa-miR-1 | MIMAT0000416 | 21169019 |
| Nervous System | hsa-miR-124-3p | MIMAT0000422 | 18577219 |
| Nervous System | hsa-miR-124-3p | MIMAT0000422 | 23318130 |
| Nervous System | hsa-miR-125a-5p | MIMAT0000443 | 21220473 |
| Nervous System | hsa-miR-128-3p | MIMAT0000424 | 21143953 |
| Nervous System | hsa-miR-133b | MIMAT0000770 | 17761882 |
| Nervous System | hsa-miR-137 | MIMAT0000429 | 18577219 |
| Nervous System | hsa-miR-145-5p | MIMAT0000437 | 21276775 |
| Nervous System | hsa-miR-151a-3p | MIMAT0000757 | 21143953 |
| Nervous System | hsa-miR-152-3p | MIMAT0000438 | 20841484 |
| Nervous System | hsa-miR-184 | MIMAT0000454 | 20409325 |
| Nervous System | hsa-miR-185-5p | MIMAT0000455 | 21143953 |
| Nervous System | hsa-miR-18a-5p | MIMAT0000072 | 20080637 |
| Nervous System | hsa-miR-19a-3p | MIMAT0000073 | 20080637 |
| Nervous System | hsa-miR-19b-3p | MIMAT0000074 | 20435064 |
| Nervous System | hsa-miR-214-3p | MIMAT0000271 | 21276775 |
| Nervous System | hsa-miR-29a-3p | MIMAT0000086 | 19584290 |
| Nervous System | hsa-miR-29a-3p | MIMAT0000086 | 21169019 |
| Nervous System | hsa-miR-302b-5p | MIMAT0000714 | 20435064 |
| Nervous System | hsa-miR-323a-3p | MIMAT0000755 | 20435064 |
| Nervous System | hsa-miR-326 | MIMAT0000756 | 19955368 |
| Nervous System | hsa-miR-34a-5p | MIMAT0000255 | 21266077 |
| Nervous System | hsa-miR-509-3p | MIMAT0002881 | 21143953 |
| Nervous System | mml-miR-219 | MIMAT0002575 | 17553428 |
| Nervous System | mmu-miR-124-3p | MIMAT0000134 | 18577219 |
| Nervous System | mmu-miR-124-3p | MIMAT0000134 | 17679093 |
| Nervous System | mmu-miR-124-3p | MIMAT0000134 | 21131957 |
| Nervous System | mmu-miR-124-3p | MIMAT0000134 | 18619591 |
| Nervous System | mmu-miR-132-3p | MIMAT0000144 | 17553428 |
| Nervous System | mmu-miR-134-5p | MIMAT0000146 | 20622856 |
| Nervous System | mmu-miR-137-3p | MIMAT0000149 | 18577219 |
| Nervous System | mmu-miR-137-5p | MIMAT0016986 | 18577219 |
| Nervous System | mmu-miR-155-5p | MIMAT0000165 | 21247879 |
| Nervous System | mmu-miR-219a-5p | MIMAT0000664 | 17553428 |
| Nervous System | mmu-miR-23a-3p | MIMAT0000532 | 19259393 |
| Nervous System | mmu-miR-23b-3p | MIMAT0000125 | 19259393 |
| Nervous System | mmu-miR-298-5p | MIMAT0000376 | 21247879 |
| Nervous System | mmu-miR-29b-3p | MIMAT0000127 | 21245165 |
| Nervous System | mmu-miR-351-5p | MIMAT0000609 | 21247879 |
| Nervous System | mmu-miR-9-5p | MIMAT0000142 | 21238922 |
| Nervous System | ppy-miR-132 | MIMAT0015755 | 17553428 |
| Nervous System | ppy-miR-219 | MIMAT0002577 | 17553428 |
| Nervous System | rno-miR-134-5p | MIMAT0000840 | 16421561 |
| Nervous System | rno-miR-140-3p | MIMAT0000574 | 18845019 |
| Nervous System | rno-miR-34a-5p | MIMAT0000815 | 18704095 |
| Nervous System | ssc-miR-132 | MIMAT0025361 | 17553428 |
| Nervous System | ssc-miR-219 | MIMAT0020590 | 17553428 |
| Nervous System | xtr-miR-132 | MIMAT0003594 | 17553428 |
| Nervous System | xtr-miR-219 | MIMAT0003632 | 17553428 |
| Neural Crest | hsa-miR-18a-5p | MIMAT0000072 | 20080637 |
| Neural Crest | hsa-miR-19a-3p | MIMAT0000073 | 20080637 |
| Neural Crest | mmu-miR-143-3p | MIMAT0000247 | 19578358 |
| Neural Crest | mmu-miR-145a-5p | MIMAT0000157 | 19578358 |
| Neural Plate | hsa-miR-302a-3p | MIMAT0000684 | 19386261 |
| Neural Stem Cells | hsa-let-7b-5p | MIMAT0000063 | 20133835 |
| Neural Stem Cells | hsa-miR-124-3p | MIMAT0000422 | 18577219 |
| Neural Stem Cells | hsa-miR-128-3p | MIMAT0000424 | 19010882 |
| Neural Stem Cells | hsa-miR-137 | MIMAT0000429 | 18577219 |
| Neural Stem Cells | hsa-miR-9-5p | MIMAT0000441 | 19330006 |
| Neural Stem Cells | mmu-let-7b-5p | MIMAT0000522 | 20133835 |
| Neural Stem Cells | mmu-miR-124-3p | MIMAT0000134 | 18577219 |
| Neural Stem Cells | mmu-miR-137-3p | MIMAT0000149 | 20368621 |
| Neural Stem Cells | mmu-miR-137-3p | MIMAT0000149 | 18577219 |
| Neural Stem Cells | mmu-miR-137-5p | MIMAT0016986 | 18577219 |
| Neural Stem Cells | mmu-miR-146a-5p | MIMAT0000158 | 21730286 |
| Neurites | hsa-miR-141-3p | MIMAT0000432 | 20584986 |
| Neurites | hsa-miR-145-5p | MIMAT0000437 | 21276775 |
| Neurites | hsa-miR-18a-5p | MIMAT0000072 | 20080637 |
| Neurites | hsa-miR-19a-3p | MIMAT0000073 | 20080637 |
| Neurites | hsa-miR-205-5p | MIMAT0000266 | 23125283 |
| Neurites | hsa-miR-21-5p | MIMAT0000076 | 20584986 |
| Neurites | hsa-miR-214-3p | MIMAT0000271 | 21276775 |
| Neurites | hsa-miR-375 | MIMAT0000728 | 20584986 |
| Neurites | mmu-miR-141-3p | MIMAT0000153 | 20584986 |
| Neurites | mmu-miR-205-5p | MIMAT0000238 | 23125283 |
| Neurites | mmu-miR-375-3p | MIMAT0000739 | 20584986 |
| Neurites | rno-miR-141-3p | MIMAT0000846 | 20584986 |
| Neurites | rno-miR-21-5p | MIMAT0000790 | 20584986 |
| Neuroendocrine Cells | mmu-miR-17-5p | MIMAT0000649 | 17765889 |
| Neurofibrillary Tangles | mmu-miR-135a-5p | MIMAT0000147 | 20619502 |
| Neurofibrillary Tangles | mmu-miR-16-5p | MIMAT0000527 | 20619502 |
| Neurofibrillary Tangles | mmu-miR-195a-5p | MIMAT0000225 | 22721728 |
| Neurofibrillary Tangles | mmu-miR-20a-5p | MIMAT0000529 | 20619502 |
| Neuroglia | hsa-miR-125b-5p | MIMAT0000423 | 20347935 |
| Neuroglia | mmu-miR-155-5p | MIMAT0000165 | 21247879 |
| Neuroglia | mmu-miR-298-5p | MIMAT0000376 | 21247879 |
| Neuroglia | mmu-miR-351-5p | MIMAT0000609 | 21247879 |
| Neurons | hsa-let-7a-5p | MIMAT0000062 | 19110058 |
| Neurons | hsa-let-7d-5p | MIMAT0000065 | 19110058 |
| Neurons | hsa-miR-101-3p | MIMAT0000099 | 20395292 |
| Neurons | hsa-miR-101-3p | MIMAT0000099 | 21172309 |
| Neurons | hsa-miR-106a-5p | MIMAT0000103 | 19110058 |
| Neurons | hsa-miR-106b-5p | MIMAT0000680 | 19110058 |
| Neurons | hsa-miR-107 | MIMAT0000104 | 20489155 |
| Neurons | hsa-miR-10a-5p | MIMAT0000253 | 20506192 |
| Neurons | hsa-miR-124-3p | MIMAT0000422 | 23318130 |
| Neurons | hsa-miR-125a-5p | MIMAT0000443 | 16227573 |
| Neurons | hsa-miR-125b-5p | MIMAT0000423 | 20159450 |
| Neurons | hsa-miR-125b-5p | MIMAT0000423 | 16227573 |
| Neurons | hsa-miR-128-3p | MIMAT0000424 | 21143953 |
| Neurons | hsa-miR-130a-3p | MIMAT0000425 | 19110058 |
| Neurons | hsa-miR-132-3p | MIMAT0000426 | 18577589 |
| Neurons | hsa-miR-132-3p | MIMAT0000426 | 18082412 |
| Neurons | hsa-miR-133b | MIMAT0000770 | 19695767 |
| Neurons | hsa-miR-133b | MIMAT0000770 | 17761882 |
| Neurons | hsa-miR-137 | MIMAT0000429 | 22003227 |
| Neurons | hsa-miR-145-5p | MIMAT0000437 | 21276775 |
| Neurons | hsa-miR-151a-3p | MIMAT0000757 | 21143953 |
| Neurons | hsa-miR-15a-5p | MIMAT0000068 | 19110058 |
| Neurons | hsa-miR-17-5p | MIMAT0000070 | 19110058 |
| Neurons | hsa-miR-185-5p | MIMAT0000455 | 21143953 |
| Neurons | hsa-miR-205-5p | MIMAT0000266 | 23125283 |
| Neurons | hsa-miR-20a-5p | MIMAT0000075 | 19110058 |
| Neurons | hsa-miR-21-5p | MIMAT0000076 | 21170291 |
| Neurons | hsa-miR-214-3p | MIMAT0000271 | 21276775 |
| Neurons | hsa-miR-30a-5p | MIMAT0000087 | 18632683 |
| Neurons | hsa-miR-509-3p | MIMAT0002881 | 21143953 |
| Neurons | hsa-miR-7-5p | MIMAT0000252 | 19628698 |
| Neurons | hsa-miR-9-3p | MIMAT0000442 | 19118166 |
| Neurons | hsa-miR-9-5p | MIMAT0000441 | 19118166 |
| Neurons | mmu-miR-124-3p | MIMAT0000134 | 19287386 |
| Neurons | mmu-miR-124-3p | MIMAT0000134 | 17679093 |
| Neurons | mmu-miR-124-3p | MIMAT0000134 | 18619591 |
| Neurons | mmu-miR-125b-5p | MIMAT0000136 | 16227573 |
| Neurons | mmu-miR-134-5p | MIMAT0000146 | 20126250 |
| Neurons | mmu-miR-16-5p | MIMAT0000527 | 20847275 |
| Neurons | mmu-miR-205-5p | MIMAT0000238 | 23125283 |
| Neurons | mmu-miR-206-3p | MIMAT0000239 | 20007902 |
| Neurons | mmu-miR-29b-3p | MIMAT0000127 | 21245165 |
| Neurons | mmu-miR-9-5p | MIMAT0000142 | 21238922 |
| Neurons | rno-miR-1-3p | MIMAT0003125 | 16260724 |
| Neurons | rno-miR-132-3p | MIMAT0000838 | 17994015 |
| Neurons | rno-miR-132-3p | MIMAT0000838 | 19958814 |
| Neurons | rno-miR-132-3p | MIMAT0000838 | 16260724 |
| Neurons | rno-miR-133b-3p | MIMAT0003126 | 19695767 |
| Neurons | rno-miR-134-5p | MIMAT0000840 | 16421561 |
| Neurons | rno-miR-134-5p | MIMAT0000840 | 19197241 |
| Neurons | rno-miR-181a-5p | MIMAT0000858 | 22144581 |
| Neurons | rno-miR-21-5p | MIMAT0000790 | 20840605 |
| Neurons | rno-miR-338-3p | MIMAT0000581 | 19020050 |
| Neurons | rno-miR-92a-3p | MIMAT0000816 | 20050974 |
| Neutrophils | hsa-miR-146a-5p | MIMAT0000449 | 20375304 |
| Neutrophils | hsa-miR-155-5p | MIMAT0000646 | 19289835 |
| Neutrophils | hsa-miR-223-3p | MIMAT0000280 | 18278031 |
| Neutrophils | hsa-miR-9-5p | MIMAT0000441 | 19289835 |
| Neutrophils | mmu-miR-223-3p | MIMAT0000665 | 18278031 |
| NIH 3T3 Cells | mmu-miR-298-5p | MIMAT0000376 | 18986979 |
| NIH 3T3 Cells | mmu-miR-328-3p | MIMAT0000565 | 18986979 |
| NIH 3T3 Cells | rno-miR-335 | MIMAT0000575 | 20933506 |
| Nuclear Envelope | mmu-miR-23a-3p | MIMAT0000532 | 19259393 |
| Nuclear Envelope | mmu-miR-23b-3p | MIMAT0000125 | 19259393 |
| Nuclear Envelope | mmu-miR-31-5p | MIMAT0000538 | 20081371 |
| Nuclear Lamina | mmu-miR-31-5p | MIMAT0000538 | 20081371 |
| Nuclear Pore | mmu-miR-23a-3p | MIMAT0000532 | 19259393 |
| Nuclear Pore | mmu-miR-23b-3p | MIMAT0000125 | 19259393 |
| Nucleus Accumbens | rno-miR-181a-5p | MIMAT0000858 | 22144581 |
| Odontoblasts | hsa-miR-338-3p | MIMAT0000763 | 23380982 |
| Oligodendroglia | hsa-miR-122-5p | MIMAT0000421 | 23348614 |
| Oligodendroglia | hsa-miR-206 | MIMAT0000462 | 19606501 |
| Oligodendroglia | mmu-miR-206-3p | MIMAT0000239 | 19606501 |
| Oligodendroglia | mmu-miR-219a-5p | MIMAT0000664 | 20223197 |
| Oligodendroglia | mmu-miR-219a-5p | MIMAT0000664 | 20223198 |
| Oligodendroglia | mmu-miR-23a-3p | MIMAT0000532 | 19259393 |
| Oligodendroglia | mmu-miR-23b-3p | MIMAT0000125 | 19259393 |
| Oligodendroglia | rno-miR-206-3p | MIMAT0000879 | 19606501 |
| Oligodendroglia | rno-miR-9a-5p | MIMAT0000781 | 18987208 |
| Oocytes | hsa-miR-224-5p | MIMAT0000281 | 20118412 |
| Oocytes | hsa-miR-302b-3p | MIMAT0000715 | 19342891 |
| Oocytes | mmu-miR-503-5p | MIMAT0003188 | 19799966 |
| Organelles | hsa-miR-320a | MIMAT0000510 | 18523662 |
| Osteoblasts | hsa-miR-125a-5p | MIMAT0000443 | 19011087 |
| Osteoblasts | hsa-miR-125b-5p | MIMAT0000423 | 19011087 |
| Osteoblasts | hsa-miR-199a-5p | MIMAT0000231 | 19011087 |
| Osteoblasts | hsa-miR-199a-5p | MIMAT0000231 | 22441842 |
| Osteoblasts | hsa-miR-26a-5p | MIMAT0000082 | 18197755 |
| Osteoblasts | hsa-miR-26a-5p | MIMAT0000082 | 19011087 |
| Osteoblasts | hsa-miR-29b-3p | MIMAT0000100 | 23478036 |
| Osteoblasts | hsa-miR-30c-5p | MIMAT0000244 | 23469762 |
| Osteoblasts | hsa-miR-346 | MIMAT0000773 | 19011087 |
| Osteoblasts | hsa-miR-34b-3p | MIMAT0004676 | 22498974 |
| Osteoblasts | hsa-miR-34c-5p | MIMAT0000686 | 22498974 |
| Osteoblasts | hsa-miR-433-3p | MIMAT0001627 | 23353875 |
| Osteoblasts | hsa-miR-637 | MIMAT0003307 | 21880893 |
| Osteoblasts | mmu-miR-206-3p | MIMAT0000239 | 19933329 |
| Osteoblasts | mmu-miR-2861 | MIMAT0013803 | 19920351 |
| Osteoblasts | mmu-miR-370-3p | MIMAT0001095 | 22587915 |
| Osteoblasts | mmu-miR-3960 | MIMAT0019336 | 21324897 |
| Osteoclasts | hsa-miR-148b-3p | MIMAT0000759 | 23238785 |
| Osteoclasts | hsa-miR-34b-3p | MIMAT0004676 | 22498974 |
| Osteoclasts | hsa-miR-34c-5p | MIMAT0000686 | 22498974 |
| Ovary | hsa-miR-101-3p | MIMAT0000099 | 17875710 |
| Ovary | hsa-miR-125a-5p | MIMAT0000443 | 21220473 |
| Ovary | hsa-miR-140-5p | MIMAT0000431 | 17875710 |
| Ovary | hsa-miR-141-3p | MIMAT0000432 | 19854497 |
| Ovary | hsa-miR-141-3p | MIMAT0000432 | 17875710 |
| Ovary | hsa-miR-143-3p | MIMAT0000435 | 17875710 |
| Ovary | hsa-miR-199a-5p | MIMAT0000231 | 17875710 |
| Ovary | hsa-miR-200a-3p | MIMAT0000682 | 17875710 |
| Ovary | hsa-miR-200a-3p | MIMAT0000682 | 19854497 |
| Ovary | hsa-miR-200b-3p | MIMAT0000318 | 17875710 |
| Ovary | hsa-miR-200b-3p | MIMAT0000318 | 19854497 |
| Ovary | hsa-miR-200c-3p | MIMAT0000617 | 17875710 |
| Ovary | hsa-miR-200c-3p | MIMAT0000617 | 19854497 |
| Ovary | hsa-miR-212-3p | MIMAT0000269 | 17875710 |
| Ovary | hsa-miR-29c-3p | MIMAT0000681 | 21436257 |
| Ovary | hsa-miR-429 | MIMAT0001536 | 19854497 |
| Ovary | hsa-miR-9-5p | MIMAT0000441 | 19702828 |
| Ovary | mmu-miR-466h-5p | MIMAT0004884 | 21337326 |
| Ovary | mmu-miR-503-5p | MIMAT0003188 | 19799966 |
| Palate | hsa-miR-140-5p | MIMAT0000431 | 22012839 |
| Pancreas | hsa-miR-143-3p | MIMAT0000435 | 16966691 |
| Pancreas | hsa-miR-182-5p | MIMAT0000259 | 19569050 |
| Pancreas | hsa-miR-194-5p | MIMAT0000460 | 19569050 |
| Pancreas | hsa-miR-197-3p | MIMAT0000227 | 23139153 |
| Pancreas | hsa-miR-200b-3p | MIMAT0000318 | 19569050 |
| Pancreas | hsa-miR-200c-3p | MIMAT0000617 | 19569050 |
| Pancreas | hsa-miR-21-5p | MIMAT0000076 | 18372920 |
| Pancreas | hsa-miR-21-5p | MIMAT0000076 | 16966691 |
| Pancreas | hsa-miR-26b-5p | MIMAT0000083 | 19569050 |
| Pancreas | hsa-miR-298 | MIMAT0004901 | 18642050 |
| Pancreas | hsa-miR-30a-5p | MIMAT0000087 | 23338554 |
| Pancreas | hsa-miR-34a-5p | MIMAT0000255 | 21240262 |
| Pancreas | hsa-miR-374a-5p | MIMAT0000727 | 19569050 |
| Pancreas | hsa-miR-429 | MIMAT0001536 | 19569050 |
| Pancreas | hsa-miR-520h | MIMAT0002867 | 20628378 |
| Pancreas | mmu-miR-124-3p | MIMAT0000134 | 17462994 |
| Pancreas | mmu-miR-19b-3p | MIMAT0000513 | 21781967 |
| Pancreas | mmu-miR-21a-5p | MIMAT0000530 | 18372920 |
| Pancreatic Ducts | hsa-miR-146a-5p | MIMAT0000449 | 20124483 |
| PC12 Cells | hsa-miR-15a-5p | MIMAT0000068 | 20621999 |
| PC12 Cells | hsa-miR-16-5p | MIMAT0000069 | 20621999 |
| PC12 Cells | hsa-miR-221-3p | MIMAT0000278 | 19438724 |
| PC12 Cells | hsa-miR-222-3p | MIMAT0000279 | 19438724 |
| PC12 Cells | rno-miR-140-3p | MIMAT0000574 | 18845019 |
| PC12 Cells | rno-miR-221-3p | MIMAT0000890 | 19438724 |
| PC12 Cells | rno-miR-222-3p | MIMAT0000891 | 19438724 |
| Pericardium | rno-miR-31a-5p | MIMAT0000810 | 23270756 |
| Philadelphia Chromosome | hsa-miR-203a | MIMAT0000264 | 18538733 |
| Photoreceptor Cells | hsa-miR-182-5p | MIMAT0000259 | 17597072 |
| Vertebrate | | | |
| Photoreceptor Cells | hsa-miR-96-5p | MIMAT0000095 | 17597072 |
| Vertebrate | | | |
| Placenta | hsa-miR-106a-5p | MIMAT0000103 | 23438603 |
| Placenta | hsa-miR-125b-5p | MIMAT0000423 | 21703189 |
| Placenta | hsa-miR-155-5p | MIMAT0000646 | 20452491 |
| Placenta | hsa-miR-195-5p | MIMAT0000461 | 20952681 |
| Placenta | hsa-miR-19b-3p | MIMAT0000074 | 23438603 |
| Plasma | hsa-let-7a-5p | MIMAT0000062 | 19939273 |
| Plasma | hsa-let-7c-3p | MIMAT0026472 | 19939273 |
| Plasma | hsa-let-7c-5p | MIMAT0000064 | 19939273 |
| Plasma | hsa-let-7d-3p | MIMAT0004484 | 19939273 |
| Plasma | hsa-let-7d-5p | MIMAT0000065 | 19939273 |
| Plasma | hsa-let-7e-3p | MIMAT0004485 | 19939273 |
| Plasma | hsa-let-7e-5p | MIMAT0000066 | 19939273 |
| Plasma | hsa-let-7g-3p | MIMAT0004584 | 19939273 |
| Plasma | hsa-let-7g-5p | MIMAT0000414 | 19939273 |
| Plasma | hsa-let-7i-3p | MIMAT0004585 | 19939273 |
| Plasma | hsa-let-7i-5p | MIMAT0000415 | 19939273 |
| Plasma | hsa-miR-1 | MIMAT0000416 | 23141496 |
| Plasma | hsa-miR-106a-5p | MIMAT0000103 | 20385818 |
| Plasma | hsa-miR-106b-5p | MIMAT0000680 | 20385818 |
| Plasma | hsa-miR-106b-5p | MIMAT0000680 | 18728182 |
| Plasma | hsa-miR-122-5p | MIMAT0000421 | 16459310 |
| Plasma | hsa-miR-125b-5p | MIMAT0000423 | 20497960 |
| Plasma | hsa-miR-145-5p | MIMAT0000437 | 23201159 |
| Plasma | hsa-miR-181a-5p | MIMAT0000256 | 18728182 |
| Plasma | hsa-miR-181b-5p | MIMAT0000257 | 18728182 |
| Plasma | hsa-miR-19a-3p | MIMAT0000073 | 18728182 |
| Plasma | hsa-miR-19b-3p | MIMAT0000074 | 18728182 |
| Plasma | hsa-miR-221-3p | MIMAT0000278 | 21487968 |
| Plasma | hsa-miR-25-3p | MIMAT0000081 | 18728182 |
| Plasma | hsa-miR-32-5p | MIMAT0000090 | 18728182 |
| Plasma | hsa-miR-33a-5p | MIMAT0000091 | 20466882 |
| Plasma | hsa-miR-33a-5p | MIMAT0000091 | 20466885 |
| Plasma | hsa-miR-33b-5p | MIMAT0003301 | 20466882 |
| Plasma | hsa-miR-503-5p | MIMAT0002874 | 21220732 |
| Plasma | hsa-miR-92a-3p | MIMAT0000092 | 18728182 |
| Plasma | hsa-miR-93-5p | MIMAT0000093 | 18728182 |
| Plasma | mmu-miR-122-5p | MIMAT0000246 | 21364282 |
| Plasma | mmu-miR-122-5p | MIMAT0000246 | 16459310 |
| Plasma | mmu-miR-122-5p | MIMAT0000246 | 18158304 |
| Plasma | mmu-miR-122-5p | MIMAT0000246 | 16258535 |
| Plasma | mmu-miR-155-5p | MIMAT0000165 | 18055230 |
| Plasma | mmu-miR-33-5p | MIMAT0000667 | 20466885 |
| Plasma Cells | hsa-miR-106b-5p | MIMAT0000680 | 18728182 |
| Plasma Cells | hsa-miR-125b-5p | MIMAT0000423 | 20497960 |
| Plasma Cells | hsa-miR-181a-5p | MIMAT0000256 | 18728182 |
| Plasma Cells | hsa-miR-181b-5p | MIMAT0000257 | 18728182 |
| Plasma Cells | hsa-miR-19a-3p | MIMAT0000073 | 18728182 |
| Plasma Cells | hsa-miR-19b-3p | MIMAT0000074 | 18728182 |
| Plasma Cells | hsa-miR-25-3p | MIMAT0000081 | 18728182 |
| Plasma Cells | hsa-miR-32-5p | MIMAT0000090 | 18728182 |
| Plasma Cells | hsa-miR-92a-3p | MIMAT0000092 | 18728182 |
| Plasma Cells | hsa-miR-93-5p | MIMAT0000093 | 18728182 |
| Pluripotent Stem Cells | hsa-miR-124-3p | MIMAT0000422 | 20362537 |
| Pluripotent Stem Cells | hsa-miR-302a-3p | MIMAT0000684 | 21062975 |
| Pluripotent Stem Cells | hsa-miR-302a-3p | MIMAT0000684 | 23185040 |
| Pluripotent Stem Cells | hsa-miR-302b-3p | MIMAT0000715 | 21062975 |
| Pluripotent Stem Cells | hsa-miR-302b-3p | MIMAT0000715 | 23185040 |
| Pluripotent Stem Cells | hsa-miR-302c-3p | MIMAT0000717 | 21062975 |
| Pluripotent Stem Cells | hsa-miR-302c-3p | MIMAT0000717 | 23185040 |
| Pluripotent Stem Cells | hsa-miR-302d-3p | MIMAT0000718 | 21062975 |
| Pluripotent Stem Cells | hsa-miR-302d-3p | MIMAT0000718 | 23185040 |
| Pluripotent Stem Cells | hsa-miR-9-5p | MIMAT0000441 | 20362537 |
| Pluripotent Stem Cells | mmu-let-7c-5p | MIMAT0000523 | 20054295 |
| Pluripotent Stem Cells | mmu-miR-106b-5p | MIMAT0000386 | 21285944 |
| Pluripotent Stem Cells | mmu-miR-9-5p | MIMAT0000142 | 20362537 |
| Pluripotent Stem Cells | mmu-miR-93-5p | MIMAT0000540 | 21285944 |
| Podocytes | mmu-miR-29c-3p | MIMAT0000536 | 21310958 |
| Podocytes | rno-miR-192-5p | MIMAT0000867 | 20393144 |
| Podocytes | rno-miR-215 | MIMAT0003118 | 20393144 |
| Polyribosomes | hsa-miR-122-5p | MIMAT0000421 | 16777601 |
| Polyribosomes | hsa-miR-30a-3p | MIMAT0000088 | 16239240 |
| Polyribosomes | hsa-miR-519a-3p | MIMAT0002869 | 19088191 |
| Polyribosomes | hsa-miR-519b-3p | MIMAT0002837 | 19088191 |
| Polyribosomes | hsa-miR-519c-3p | MIMAT0002832 | 19088191 |
| Polyribosomes | mmu-miR-23b-3p | MIMAT0000125 | 18716031 |
| Portal Vein | hsa-miR-328-3p | MIMAT0000752 | 18560585 |
| Portal Vein | hsa-miR-328-5p | MIMAT0026486 | 18560585 |
| Precursor Cells T-Lymphoid | hsa-miR-222-3p | MIMAT0000279 | 23522449 |
| Prefrontal Cortex | hsa-miR-219a-5p | MIMAT0000276 | 19196972 |
| Prefrontal Cortex | hsa-miR-30a-5p | MIMAT0000087 | 18632683 |
| Prefrontal Cortex | mmu-miR-219a-5p | MIMAT0000664 | 19196972 |
| Prefrontal Cortex | rno-let-7d-5p | MIMAT0000562 | 20557304 |
| Prosencephalon | hsa-miR-132-3p | MIMAT0000426 | 18082412 |
| Prosencephalon | mmu-miR-9-5p | MIMAT0000142 | 21238922 |
| Prostate | hsa-let-7a-5p | MIMAT0000062 | 20418948 |
| Prostate | hsa-miR-101-3p | MIMAT0000099 | 20478051 |
| Prostate | hsa-miR-101-3p | MIMAT0000099 | 19008416 |
| Prostate | hsa-miR-106a-5p | MIMAT0000103 | 16461460 |
| Prostate | hsa-miR-106b-5p | MIMAT0000680 | 18676839 |
| Prostate | hsa-miR-107 | MIMAT0000104 | 20884628 |
| Prostate | hsa-miR-122-5p | MIMAT0000421 | 20884628 |
| Prostate | hsa-miR-125b-5p | MIMAT0000423 | 17891175 |
| Prostate | hsa-miR-125b-5p | MIMAT0000423 | 18056640 |
| Prostate | hsa-miR-126-3p | MIMAT0000445 | 18193184 |
| Prostate | hsa-miR-126-5p | MIMAT0000444 | 18193184 |
| Prostate | hsa-miR-1296-5p | MIMAT0005794 | 20332239 |
| Prostate | hsa-miR-138-5p | MIMAT0000430 | 18193184 |
| Prostate | hsa-miR-141-3p | MIMAT0000432 | 21224847 |
| Prostate | hsa-miR-143-3p | MIMAT0000435 | 20353999 |
| Prostate | hsa-miR-145-5p | MIMAT0000437 | 23355420 |
| Prostate | hsa-miR-145-5p | MIMAT0000437 | 23480797 |
| Prostate | hsa-miR-145-5p | MIMAT0000437 | 21360565 |
| Prostate | hsa-miR-145-5p | MIMAT0000437 | 20353999 |
| Prostate | hsa-miR-145-5p | MIMAT0000437 | 19996289 |
| Prostate | hsa-miR-146a-5p | MIMAT0000449 | 18174313 |
| Prostate | hsa-miR-146a-5p | MIMAT0000449 | 22161865 |
| Prostate | hsa-miR-16-5p | MIMAT0000069 | 19738602 |
| Prostate | hsa-miR-17-3p | MIMAT0000071 | 19771525 |
| Prostate | hsa-miR-17-3p | MIMAT0000071 | 21203553 |
| Prostate | hsa-miR-17-5p | MIMAT0000070 | 19771525 |
| Prostate | hsa-miR-182-5p | MIMAT0000259 | 19574223 |
| Prostate | hsa-miR-185-5p | MIMAT0000455 | 23417242 |
| Prostate | hsa-miR-199b-5p | MIMAT0000263 | 23354452 |
| Prostate | hsa-miR-200c-3p | MIMAT0000617 | 21224847 |
| Prostate | hsa-miR-205-5p | MIMAT0000266 | 20737563 |
| Prostate | hsa-miR-20a-5p | MIMAT0000075 | 16461460 |
| Prostate | hsa-miR-21-5p | MIMAT0000076 | 21317927 |
| Prostate | hsa-miR-21-5p | MIMAT0000076 | 19302977 |
| Prostate | hsa-miR-21-5p | MIMAT0000076 | 20813833 |
| Prostate | hsa-miR-21-5p | MIMAT0000076 | 21826097 |
| Prostate | hsa-miR-21-5p | MIMAT0000076 | 18372920 |
| Prostate | hsa-miR-21-5p | MIMAT0000076 | 19578724 |
| Prostate | hsa-miR-211-5p | MIMAT0000268 | 21072171 |
| Prostate | hsa-miR-217 | MIMAT0000274 | 19008416 |
| Prostate | hsa-miR-221-3p | MIMAT0000278 | 18834855 |
| Prostate | hsa-miR-221-3p | MIMAT0000278 | 21487968 |
| Prostate | hsa-miR-224-5p | MIMAT0000281 | 20180642 |
| Prostate | hsa-miR-26a-5p | MIMAT0000082 | 16461460 |
| Prostate | hsa-miR-26a-5p | MIMAT0000082 | 20478051 |
| Prostate | hsa-miR-27a-3p | MIMAT0000084 | 19574223 |
| Prostate | hsa-miR-29b-3p | MIMAT0000100 | 20657750 |
| Prostate | hsa-miR-32-5p | MIMAT0000090 | 18676839 |
| Prostate | hsa-miR-330-3p | MIMAT0000751 | 19597470 |
| Prostate | hsa-miR-331-3p | MIMAT0000760 | 19996289 |
| Prostate | hsa-miR-34a-5p | MIMAT0000255 | 20433755 |
| Prostate | hsa-miR-34a-5p | MIMAT0000255 | 21240262 |
| Prostate | hsa-miR-34a-5p | MIMAT0000255 | 18834855 |
| Prostate | hsa-miR-377-3p | MIMAT0000730 | 21203553 |
| Prostate | hsa-miR-449a | MIMAT0001541 | 19252524 |
| Prostate | hsa-miR-449a | MIMAT0001541 | 20948989 |
| Prostate | hsa-miR-521 | MIMAT0002854 | 18668526 |
| Prostate | hsa-miR-616-3p | MIMAT0004805 | 21224345 |
| Prostate | hsa-miR-616-5p | MIMAT0003284 | 21224345 |
| Prostate | hsa-miR-96-5p | MIMAT0000095 | 19574223 |
| Prostate | mmu-miR-21a-5p | MIMAT0000530 | 18372920 |
| Pseudopodia | hsa-miR-10b-5p | MIMAT0000254 | 22573479 |
| Pseudopodia | hsa-miR-124-3p | MIMAT0000422 | 21672940 |
| Pulmonary Artery | hsa-miR-21-5p | MIMAT0000076 | 20693317 |
| Raphe Nuclei | mmu-miR-16-5p | MIMAT0000527 | 20847275 |
| Reed-Sternberg Cells | hsa-let-7a-5p | MIMAT0000062 | 18583325 |
| Reed-Sternberg Cells | hsa-miR-9-5p | MIMAT0000441 | 18583325 |
| Reticulocytes | hsa-miR-320a | MIMAT0000510 | 18523662 |
| Retina | hsa-miR-124-3p | MIMAT0000422 | 17724173 |
| Retina | hsa-miR-182-5p | MIMAT0000259 | 17597072 |
| Retina | hsa-miR-204-5p | MIMAT0000265 | 20056717 |
| Retina | hsa-miR-204-5p | MIMAT0000265 | 20713703 |
| Retina | hsa-miR-21-5p | MIMAT0000076 | 20056717 |
| Retina | hsa-miR-29a-3p | MIMAT0000086 | 17724173 |
| Retina | hsa-miR-29b-3p | MIMAT0000100 | 17724173 |
| Retina | hsa-miR-96-5p | MIMAT0000095 | 17597072 |
| Retina | mmu-miR-150-5p | MIMAT0000160 | 18500251 |
| Retina | mmu-miR-184-3p | MIMAT0000213 | 18500251 |
| Retina | mmu-miR-31-5p | MIMAT0000538 | 18500251 |
| Retinal Pigment Epithelium | hsa-miR-204-5p | MIMAT0000265 | 20056717 |
| Retinal Pigment Epithelium | hsa-miR-21-5p | MIMAT0000076 | 20056717 |
| Rhombencephalon | mmu-miR-9-5p | MIMAT0000142 | 21238922 |
| Saphenous Vein | hsa-miR-503-5p | MIMAT0002874 | 21220732 |
| Sarcolemma | hsa-miR-206 | MIMAT0000462 | 17030984 |
| Sarcolemma | mmu-miR-206-3p | MIMAT0000239 | 17030984 |
| Sarcomeres | hsa-miR-21-5p | MIMAT0000076 | 18508928 |
| Sarcomeres | mmu-miR-21a-5p | MIMAT0000530 | 18508928 |
| Sarcoplasmic Reticulum | hsa-miR-1 | MIMAT0000416 | 19131648 |
| Satellite Cells Skeletal | mmu-miR-1a-3p | MIMAT0000123 | 20819939 |
| Muscle | | | |
| Satellite Cells Skeletal | mmu-miR-206-3p | MIMAT0000239 | 20819939 |
| Muscle | | | |
| Schwann Cells | hsa-miR-10b-5p | MIMAT0000254 | 20550523 |
| Schwann Cells | hsa-miR-7-5p | MIMAT0000252 | 21156648 |
| Schwann Cells | rno-miR-29a-3p | MIMAT0000802 | 19170179 |
| Schwann Cells | rno-miR-9a-5p | MIMAT0000781 | 18987208 |
| Sciatic Nerve | hsa-miR-145-5p | MIMAT0000437 | 21276775 |
| Sciatic Nerve | hsa-miR-214-3p | MIMAT0000271 | 21276775 |
| Secretory Vesicles | hsa-miR-9-5p | MIMAT0000441 | 16831872 |
| Secretory Vesicles | rno-miR-9a-5p | MIMAT0000781 | 16831872 |
| Serum | hsa-miR-1 | MIMAT0000416 | 20458751 |
| Serum | hsa-miR-1 | MIMAT0000416 | 19775284 |
| Serum | hsa-miR-1 | MIMAT0000416 | 15951802 |
| Serum | hsa-miR-101-3p | MIMAT0000099 | 19155302 |
| Serum | hsa-miR-122-5p | MIMAT0000421 | 20351063 |
| Serum | hsa-miR-122-5p | MIMAT0000421 | 19726678 |
| Serum | hsa-miR-130a-3p | MIMAT0000425 | 17957028 |
| Serum | hsa-miR-132-3p | MIMAT0000426 | 19819989 |
| Serum | hsa-miR-133a-3p | MIMAT0000427 | 17344217 |
| Serum | hsa-miR-133b | MIMAT0000770 | 17344217 |
| Serum | hsa-miR-181a-5p | MIMAT0000256 | 22476949 |
| Serum | hsa-miR-200b-3p | MIMAT0000318 | 22144583 |
| Serum | hsa-miR-206 | MIMAT0000462 | 19620785 |
| Serum | hsa-miR-21-5p | MIMAT0000076 | 21347332 |
| Serum | hsa-miR-21-5p | MIMAT0000076 | 22956424 |
| Serum | hsa-miR-21-5p | MIMAT0000076 | 19826040 |
| Serum | hsa-miR-221-3p | MIMAT0000278 | 20516212 |
| Serum | hsa-miR-221-3p | MIMAT0000278 | 18413744 |
| Serum | hsa-miR-222-3p | MIMAT0000279 | 18413744 |
| Serum | hsa-miR-26a-5p | MIMAT0000082 | 20857419 |
| Serum | hsa-miR-29a-3p | MIMAT0000086 | 20041405 |
| Serum | hsa-miR-29b-3p | MIMAT0000100 | 20041405 |
| Serum | hsa-miR-29c-3p | MIMAT0000681 | 20041405 |
| Serum | hsa-miR-31-5p | MIMAT0000089 | 23518389 |
| Serum | hsa-miR-422a | MIMAT0001339 | 20351063 |
| Serum | mmu-let-7c-5p | MIMAT0000523 | 17438130 |
| Serum | mmu-miR-133a-3p | MIMAT0000145 | 16380711 |
| Serum | mmu-miR-133a-3p | MIMAT0000145 | 16923828 |
| Serum | mmu-miR-143-3p | MIMAT0000247 | 19578358 |
| Serum | mmu-miR-145a-5p | MIMAT0000157 | 19578358 |
| Serum | mmu-miR-1a-3p | MIMAT0000123 | 15951802 |
| Serum | mmu-miR-1a-3p | MIMAT0000123 | 16380711 |
| Serum | mmu-miR-1a-3p | MIMAT0000123 | 16923828 |
| Serum | mmu-miR-206-3p | MIMAT0000239 | 16923828 |
| Serum | mmu-miR-486-5p | MIMAT0003130 | 20142475 |
| Serum | rno-miR-1-3p | MIMAT0003125 | 19775284 |
| Serum | rno-miR-200c-3p | MIMAT0000873 | 22144583 |
| Side-Population Cells | hsa-miR-520h | MIMAT0002867 | 20628378 |
| Skin | hsa-miR-125b-5p | MIMAT0000423 | 21412257 |
| Skin | hsa-miR-155-5p | MIMAT0000646 | 20673989 |
| Skin | hsa-miR-193b-3p | MIMAT0002819 | 20304954 |
| Skin | hsa-miR-200a-3p | MIMAT0000682 | 20827281 |
| Skin | hsa-miR-203a | MIMAT0000264 | 18483491 |
| Skin | hsa-miR-203a | MIMAT0000264 | 17622355 |
| Skin | hsa-miR-203a | MIMAT0000264 | 20827281 |
| Skin | hsa-miR-203a | MIMAT0000264 | 22917968 |
| Skin | hsa-miR-203a | MIMAT0000264 | 18311128 |
| Skin | hsa-miR-21-5p | MIMAT0000076 | 19672202 |
| Skin | hsa-miR-224-5p | MIMAT0000281 | 20827281 |
| Skin | hsa-miR-26a-5p | MIMAT0000082 | 20827281 |
| Skin | hsa-miR-27b-3p | MIMAT0000419 | 20827281 |
| Skin | hsa-miR-29a-3p | MIMAT0000086 | 20201077 |
| Skin | hsa-miR-29b-3p | MIMAT0000100 | 20201077 |
| Skin | hsa-miR-302b-3p | MIMAT0000715 | 19342891 |
| Skin | hsa-miR-31-5p | MIMAT0000089 | 23233723 |
| Skin | hsa-miR-335-5p | MIMAT0000765 | 21164520 |
| Skin | hsa-miR-424-5p | MIMAT0001341 | 21179471 |
| Skin | hsa-miR-433-3p | MIMAT0001627 | 20181727 |
| Skin | hsa-miR-532-5p | MIMAT0002888 | 19336521 |
| Skin | mmu-miR-122-5p | MIMAT0000246 | 16258535 |
| Skin | mmu-miR-203-3p | MIMAT0000236 | 18311128 |
| Skin | mmu-miR-31-5p | MIMAT0000538 | 20522784 |
| Somites | mmu-miR-137-3p | MIMAT0000149 | 21256124 |
| Somites | mmu-miR-153-3p | MIMAT0000163 | 21256124 |
| Somites | mmu-miR-363-3p | MIMAT0000708 | 21256124 |
| Spheroids Cellular | hsa-miR-299-5p | MIMAT0002890 | 19538464 |
| Spinal Cord | hsa-miR-124-3p | MIMAT0000422 | 17403776 |
| Spinal Cord | hsa-miR-124-3p | MIMAT0000422 | 23318130 |
| Spinal Cord | hsa-miR-128-3p | MIMAT0000424 | 17403776 |
| Spinal Cord | mmu-miR-124-3p | MIMAT0000134 | 17403776 |
| Spinal Cord | mmu-miR-219a-5p | MIMAT0000664 | 20223198 |
| Spinal Nerve Roots | hsa-miR-145-5p | MIMAT0000437 | 21276775 |
| Spinal Nerve Roots | hsa-miR-214-3p | MIMAT0000271 | 21276775 |
| Spine | hsa-miR-10a-5p | MIMAT0000253 | 20506192 |
| Spine | hsa-miR-125b-5p | MIMAT0000423 | 20159450 |
| Spine | hsa-miR-132-3p | MIMAT0000426 | 19850129 |
| Spine | mmu-miR-134-5p | MIMAT0000146 | 20126250 |
| Spine | mmu-miR-140-5p | MIMAT0000151 | 16828749 |
| Spine | rno-miR-134-5p | MIMAT0000840 | 16421561 |
| Spine | rno-miR-181a-5p | MIMAT0000858 | 22144581 |
| Spleen | hsa-miR-34a-5p | MIMAT0000255 | 20627091 |
| Spleen | hsa-miR-7-5p | MIMAT0000252 | 18823940 |
| Spleen | mmu-miR-122-5p | MIMAT0000246 | 21364282 |
| Spleen | mmu-miR-125b-5p | MIMAT0000136 | 21118986 |
| Stem Cell Niche | mmu-miR-124-3p | MIMAT0000134 | 19287386 |
| Stem Cells | bta-miR-130a | MIMAT0009223 | 17855557 |
| Stem Cells | bta-miR-335 | MIMAT0009291 | 18185580 |
| Stem Cells | cfa-miR-130a | MIMAT0006631 | 17855557 |
| Stem Cells | cfa-miR-335 | MIMAT0006624 | 18185580 |
| Stem Cells | dre-miR-130a | MIMAT0001826 | 17855557 |
| Stem Cells | hsa-let-7a-5p | MIMAT0000062 | 19878981 |
| Stem Cells | hsa-let-7b-5p | MIMAT0000063 | 20133835 |
| Stem Cells | hsa-let-7d-5p | MIMAT0000065 | 20410187 |
| Stem Cells | hsa-miR-1 | MIMAT0000416 | 20081117 |
| Stem Cells | hsa-miR-1 | MIMAT0000416 | 20956382 |
| Stem Cells | hsa-miR-100-5p | MIMAT0000098 | 22684006 |
| Stem Cells | hsa-miR-101-3p | MIMAT0000099 | 19818710 |
| Stem Cells | hsa-miR-106a-5p | MIMAT0000103 | 17589498 |
| Stem Cells | hsa-miR-124-3p | MIMAT0000422 | 20362537 |
| Stem Cells | hsa-miR-124-3p | MIMAT0000422 | 18577219 |
| Stem Cells | hsa-miR-125a-5p | MIMAT0000443 | 20616003 |
| Stem Cells | hsa-miR-125b-5p | MIMAT0000423 | 18230348 |
| Stem Cells | hsa-miR-125b-5p | MIMAT0000423 | 18756266 |
| Stem Cells | hsa-miR-125b-5p | MIMAT0000423 | 20216554 |
| Stem Cells | hsa-miR-126-3p | MIMAT0000445 | 18474618 |
| Stem Cells | hsa-miR-126-3p | MIMAT0000445 | 18832181 |
| Stem Cells | hsa-miR-126-3p | MIMAT0000445 | 22525256 |
| Stem Cells | hsa-miR-126-3p | MIMAT0000445 | 21304604 |
| Stem Cells | hsa-miR-128-3p | MIMAT0000424 | 20574517 |
| Stem Cells | hsa-miR-128-3p | MIMAT0000424 | 19409607 |
| Stem Cells | hsa-miR-128-3p | MIMAT0000424 | 19941032 |
| Stem Cells | hsa-miR-128-3p | MIMAT0000424 | 19010882 |
| Stem Cells | hsa-miR-129-5p | MIMAT0000242 | 18189265 |
| Stem Cells | hsa-miR-130a-3p | MIMAT0000425 | 17855557 |
| Stem Cells | hsa-miR-132-3p | MIMAT0000426 | 19819989 |
| Stem Cells | hsa-miR-133a-3p | MIMAT0000427 | 23069713 |
| Stem Cells | hsa-miR-133b | MIMAT0000770 | 19695767 |
| Stem Cells | hsa-miR-135b-5p | MIMAT0000758 | 19795981 |
| Stem Cells | hsa-miR-137 | MIMAT0000429 | 18577219 |
| Stem Cells | hsa-miR-137 | MIMAT0000429 | 22003227 |
| Stem Cells | hsa-miR-138-5p | MIMAT0000430 | 20486779 |
| Stem Cells | hsa-miR-141-3p | MIMAT0000432 | 19454767 |
| Stem Cells | hsa-miR-142-3p | MIMAT0000434 | 21394831 |
| Stem Cells | hsa-miR-144-3p | MIMAT0000436 | 21285251 |
| Stem Cells | hsa-miR-145-5p | MIMAT0000437 | 19409607 |
| Stem Cells | hsa-miR-145-5p | MIMAT0000437 | 21496429 |
| Stem Cells | hsa-miR-145-5p | MIMAT0000437 | 20382729 |
| Stem Cells | hsa-miR-145-5p | MIMAT0000437 | 18474618 |
| Stem Cells | hsa-miR-145-5p | MIMAT0000437 | 20668064 |
| Stem Cells | hsa-miR-145-5p | MIMAT0000437 | 21217773 |
| Stem Cells | hsa-miR-148a-3p | MIMAT0000243 | 19435428 |
| Stem Cells | hsa-miR-153-3p | MIMAT0000439 | 20668064 |
| Stem Cells | hsa-miR-155-5p | MIMAT0000646 | 18950466 |
| Stem Cells | hsa-miR-155-5p | MIMAT0000646 | 18299402 |
| Stem Cells | hsa-miR-155-5p | MIMAT0000646 | 20550618 |
| Stem Cells | hsa-miR-15a-5p | MIMAT0000068 | 21205891 |
| Stem Cells | hsa-miR-16-5p | MIMAT0000069 | 20668064 |
| Stem Cells | hsa-miR-16-5p | MIMAT0000069 | 20216554 |
| Stem Cells | hsa-miR-16-5p | MIMAT0000069 | 21205891 |
| Stem Cells | hsa-miR-17-5p | MIMAT0000070 | 17589498 |
| Stem Cells | hsa-miR-181a-5p | MIMAT0000256 | 19585654 |
| Stem Cells | hsa-miR-181b-5p | MIMAT0000257 | 19585654 |
| Stem Cells | hsa-miR-181c-5p | MIMAT0000258 | 19585654 |
| Stem Cells | hsa-miR-181d-5p | MIMAT0002821 | 19585654 |
| Stem Cells | hsa-miR-195-5p | MIMAT0000461 | 19823043 |
| Stem Cells | hsa-miR-196a-5p | MIMAT0000226 | 19063684 |
| Stem Cells | hsa-miR-199a-5p | MIMAT0000231 | 22441842 |
| Stem Cells | hsa-miR-199a-5p | MIMAT0000231 | 19818710 |
| Stem Cells | hsa-miR-199b-5p | MIMAT0000263 | 19308264 |
| Stem Cells | hsa-miR-199b-5p | MIMAT0000263 | 23354452 |
| Stem Cells | hsa-miR-19b-3p | MIMAT0000074 | 19435428 |
| Stem Cells | hsa-miR-200a-3p | MIMAT0000682 | 21529905 |
| Stem Cells | hsa-miR-200a-3p | MIMAT0000682 | 19454767 |
| Stem Cells | hsa-miR-200a-3p | MIMAT0000682 | 20827281 |
| Stem Cells | hsa-miR-200c-3p | MIMAT0000617 | 19665978 |
| Stem Cells | hsa-miR-200c-3p | MIMAT0000617 | 21294122 |
| Stem Cells | hsa-miR-200c-3p | MIMAT0000617 | 21207483 |
| Stem Cells | hsa-miR-203a | MIMAT0000264 | 20668064 |
| Stem Cells | hsa-miR-203a | MIMAT0000264 | 18483491 |
| Stem Cells | hsa-miR-203a | MIMAT0000264 | 20827281 |
| Stem Cells | hsa-miR-203a | MIMAT0000264 | 18311128 |
| Stem Cells | hsa-miR-206 | MIMAT0000462 | 20956382 |
| Stem Cells | hsa-miR-206 | MIMAT0000462 | 17855557 |
| Stem Cells | hsa-miR-20a-5p | MIMAT0000075 | 19435428 |
| Stem Cells | hsa-miR-20a-5p | MIMAT0000075 | 17589498 |
| Stem Cells | hsa-miR-21-5p | MIMAT0000076 | 19578724 |
| Stem Cells | hsa-miR-21-5p | MIMAT0000076 | 20216554 |
| Stem Cells | hsa-miR-21-5p | MIMAT0000076 | 19342589 |
| Stem Cells | hsa-miR-21-5p | MIMAT0000076 | 22072622 |
| Stem Cells | hsa-miR-21-5p | MIMAT0000076 | 21381024 |
| Stem Cells | hsa-miR-21-5p | MIMAT0000076 | 22528454 |
| Stem Cells | hsa-miR-21-5p | MIMAT0000076 | 19816956 |
| Stem Cells | hsa-miR-21-5p | MIMAT0000076 | 23239100 |
| Stem Cells | hsa-miR-21-5p | MIMAT0000076 | 19242418 |
| Stem Cells | hsa-miR-214-3p | MIMAT0000271 | 19435428 |
| Stem Cells | hsa-miR-214-3p | MIMAT0000271 | 19818710 |
| Stem Cells | hsa-miR-216a-5p | MIMAT0000273 | 20216554 |
| Stem Cells | hsa-miR-217 | MIMAT0000274 | 20216554 |
| Stem Cells | hsa-miR-217 | MIMAT0000274 | 20668064 |
| Stem Cells | hsa-miR-221-3p | MIMAT0000278 | 20018759 |
| Stem Cells | hsa-miR-222-3p | MIMAT0000279 | 20018759 |
| Stem Cells | hsa-miR-223-3p | MIMAT0000280 | 18278031 |
| Stem Cells | hsa-miR-223-3p | MIMAT0000280 | 19278969 |
| Stem Cells | hsa-miR-224-5p | MIMAT0000281 | 20827281 |
| Stem Cells | hsa-miR-24-3p | MIMAT0000080 | 17906079 |
| Stem Cells | hsa-miR-26a-5p | MIMAT0000082 | 20827281 |
| Stem Cells | hsa-miR-26a-5p | MIMAT0000082 | 20216554 |
| Stem Cells | hsa-miR-26a-5p | MIMAT0000082 | 18197755 |
| Stem Cells | hsa-miR-26b-5p | MIMAT0000083 | 20831567 |
| Stem Cells | hsa-miR-27a-3p | MIMAT0000084 | 19440384 |
| Stem Cells | hsa-miR-27a-3p | MIMAT0000084 | 19666532 |
| Stem Cells | hsa-miR-27b-3p | MIMAT0000419 | 19666532 |
| Stem Cells | hsa-miR-27b-3p | MIMAT0000419 | 20827281 |
| Stem Cells | hsa-miR-29a-3p | MIMAT0000086 | 21665270 |
| Stem Cells | hsa-miR-29b-3p | MIMAT0000100 | 23478036 |
| Stem Cells | hsa-miR-29b-3p | MIMAT0000100 | 21665270 |
| Stem Cells | hsa-miR-302a-3p | MIMAT0000684 | 21062975 |
| Stem Cells | hsa-miR-302a-3p | MIMAT0000684 | 21266536 |
| Stem Cells | hsa-miR-302a-3p | MIMAT0000684 | 21151097 |
| Stem Cells | hsa-miR-302a-3p | MIMAT0000684 | 23185040 |
| Stem Cells | hsa-miR-302a-3p | MIMAT0000684 | 19386261 |
| Stem Cells | hsa-miR-302a-3p | MIMAT0000684 | 17855557 |
| Stem Cells | hsa-miR-302b-3p | MIMAT0000715 | 21062975 |
| Stem Cells | hsa-miR-302b-3p | MIMAT0000715 | 23185040 |
| Stem Cells | hsa-miR-302c-3p | MIMAT0000717 | 21062975 |
| Stem Cells | hsa-miR-302c-3p | MIMAT0000717 | 23185040 |
| Stem Cells | hsa-miR-302d-3p | MIMAT0000718 | 21266536 |
| Stem Cells | hsa-miR-302d-3p | MIMAT0000718 | 21062975 |
| Stem Cells | hsa-miR-302d-3p | MIMAT0000718 | 23185040 |
| Stem Cells | hsa-miR-302d-3p | MIMAT0000718 | 19229866 |
| Stem Cells | hsa-miR-31-5p | MIMAT0000089 | 21048943 |
| Stem Cells | hsa-miR-320a | MIMAT0000510 | 17855557 |
| Stem Cells | hsa-miR-324-5p | MIMAT0000761 | 18756266 |
| Stem Cells | hsa-miR-324-5p | MIMAT0000761 | 20216554 |
| Stem Cells | hsa-miR-326 | MIMAT0000756 | 23341351 |
| Stem Cells | hsa-miR-326 | MIMAT0000756 | 20667897 |
| Stem Cells | hsa-miR-326 | MIMAT0000756 | 18756266 |
| Stem Cells | hsa-miR-326 | MIMAT0000756 | 20216554 |
| Stem Cells | hsa-miR-335-5p | MIMAT0000765 | 21164520 |
| Stem Cells | hsa-miR-335-5p | MIMAT0000765 | 18185580 |
| Stem Cells | hsa-miR-34a-5p | MIMAT0000255 | 18803879 |
| Stem Cells | hsa-miR-34a-5p | MIMAT0000255 | 21240262 |
| Stem Cells | hsa-miR-34a-5p | MIMAT0000255 | 21294122 |
| Stem Cells | hsa-miR-34a-5p | MIMAT0000255 | 19773441 |
| Stem Cells | hsa-miR-34a-5p | MIMAT0000255 | 17252019 |
| Stem Cells | hsa-miR-34b-3p | MIMAT0004676 | 18803879 |
| Stem Cells | hsa-miR-34b-5p | MIMAT0000685 | 18803879 |
| Stem Cells | hsa-miR-34c-5p | MIMAT0000686 | 18803879 |
| Stem Cells | hsa-miR-372-3p | MIMAT0000724 | 19229866 |
| Stem Cells | hsa-miR-372-3p | MIMAT0000724 | 20216554 |
| Stem Cells | hsa-miR-372-3p | MIMAT0000724 | 19823043 |
| Stem Cells | hsa-miR-373-3p | MIMAT0000726 | 20216554 |
| Stem Cells | hsa-miR-375 | MIMAT0000728 | 19807270 |
| Stem Cells | hsa-miR-380-5p | MIMAT0000734 | 20871609 |
| Stem Cells | hsa-miR-433-3p | MIMAT0001627 | 23353875 |
| Stem Cells | hsa-miR-489-3p | MIMAT0002805 | 19440384 |
| Stem Cells | hsa-miR-499a-5p | MIMAT0002870 | 20081117 |
| Stem Cells | hsa-miR-499b-5p | MIMAT0019897 | 20081117 |
| Stem Cells | hsa-miR-519c-3p | MIMAT0002832 | 19825807 |
| Stem Cells | hsa-miR-520h | MIMAT0002867 | 18189265 |
| Stem Cells | hsa-miR-520h | MIMAT0002867 | 19825807 |
| Stem Cells | hsa-miR-520h | MIMAT0002867 | 19435428 |
| Stem Cells | hsa-miR-522-3p | MIMAT0002868 | 21304604 |
| Stem Cells | hsa-miR-524-5p | MIMAT0002849 | 22871495 |
| Stem Cells | hsa-miR-637 | MIMAT0003307 | 21880893 |
| Stem Cells | hsa-miR-9-5p | MIMAT0000441 | 19330006 |
| Stem Cells | hsa-miR-9-5p | MIMAT0000441 | 20362537 |
| Stem Cells | hsa-miR-92a-3p | MIMAT0000092 | 19435428 |
| Stem Cells | hsa-miR-92b-3p | MIMAT0003218 | 19544458 |
| Stem Cells | hsa-miR-98-5p | MIMAT0000096 | 19818710 |
| Stem Cells | mmu-let-7a-5p | MIMAT0000521 | 19898466 |
| Stem Cells | mmu-let-7a-5p | MIMAT0000521 | 18474618 |
| Stem Cells | mmu-let-7b-5p | MIMAT0000522 | 20133835 |
| Stem Cells | mmu-let-7c-5p | MIMAT0000523 | 20054295 |
| Stem Cells | mmu-let-7e-5p | MIMAT0000524 | 19898466 |
| Stem Cells | mmu-miR-100-3p | MIMAT0017051 | 20439489 |
| Stem Cells | mrnu-miR-100-5p | MIMAT0000655 | 20439489 |
| Stem Cells | mmu-miR-106b-5p | MIMAT0000386 | 21285944 |
| Stem Cells | mmu-miR-10a-3p | MIMAT0004659 | 20118242 |
| Stem Cells | mmu-miR-10a-5p | MIMAT0000648 | 20118242 |
| Stem Cells | mmu-miR-124-3p | MIMAT0000134 | 19287386 |
| Stem Cells | mmu-miR-124-3p | MIMAT0000134 | 18577219 |
| Stem Cells | mmu-miR-124-3p | MIMAT0000134 | 17679093 |
| Stem Cells | mmu-miR-125a-5p | MIMAT0000135 | 19898466 |
| Stem Cells | mmu-miR-125b-5p | MIMAT0000136 | 19898466 |
| Stem Cells | mmu-miR-125b-5p | MIMAT0000136 | 21118986 |
| Stem Cells | mmu-miR-125b-5p | MIMAT0000136 | 20216554 |
| Stem Cells | mmu-miR-126a-3p | MIMAT0000138 | 20216554 |
| Stem Cells | mmu-miR-126a-3p | MIMAT0000138 | 18474618 |
| Stem Cells | mmu-miR-128-3p | MIMAT0000140 | 19898466 |
| Stem Cells | mmu-miR-134-5p | MIMAT0000146 | 17916804 |
| Stem Cells | mmu-miR-134-5p | MIMAT0000146 | 18806776 |
| Stem Cells | mmu-miR-137-3p | MIMAT0000149 | 20368621 |
| Stem Cells | mmu-miR-137-3p | MIMAT0000149 | 20439489 |
| Stem Cells | mmu-miR-137-3p | MIMAT0000149 | 18577219 |
| Stem Cells | mmu-miR-137-5p | MIMAT0016986 | 20439489 |
| Stem Cells | mmu-miR-137-5p | MIMAT0016986 | 18577219 |
| Stem Cells | mrnu-miR-141-3p | MIMAT0000153 | 19454767 |
| Stem Cells | mmu-miR-143-3p | MIMAT0000247 | 19578358 |
| Stem Cells | mrnu-miR-145a-5p | MIMAT0000157 | 18474618 |
| Stem Cells | mrnu-miR-145a-5p | MIMAT0000157 | 19578358 |
| Stem Cells | mmu-miR-146a-5p | MIMAT0000158 | 21730286 |
| Stem Cells | mmu-miR-17-5p | MIMAT0000649 | 17765889 |
| Stem Cells | mmu-miR-199a-3p | MIMAT0000230 | 19251704 |
| Stem Cells | mmu-miR-19b-3p | MIMAT0000513 | 21781967 |
| Stem Cells | mmu-miR-1a-3p | MIMAT0000123 | 20819939 |
| Stem Cells | mmu-miR-1a-3p | MIMAT0000123 | 19521018 |
| Stem Cells | mmu-miR-1a-3p | MIMAT0000123 | 19933931 |
| Stem Cells | mmu-miR-1a-3p | MIMAT0000123 | 20799856 |
| Stem Cells | mmu-miR-200a-3p | MIMAT0000519 | 19454767 |
| Stem Cells | mmu-miR-203-3p | MIMAT0000236 | 18311128 |
| Stem Cells | mmu-miR-203-3p | MIMAT0000236 | 20216554 |
| Stem Cells | mmu-miR-204-5p | MIMAT0000237 | 20039258 |
| Stem Cells | mmu-miR-205-5p | MIMAT0000238 | 20103531 |
| Stem Cells | mmu-miR-206-3p | MIMAT0000239 | 20819939 |
| Stem Cells | mmu-miR-211-5p | MIMAT0000668 | 20039258 |
| Stem Cells | mmu-miR-214-3p | MIMAT0000661 | 19818710 |
| Stem Cells | mmu-miR-21a-5p | MIMAT0000530 | 20216554 |
| Stem Cells | mmu-miR-221-3p | MIMAT0000669 | 20018759 |
| Stem Cells | mmu-miR-223-3p | MIMAT0000665 | 18278031 |
| Stem Cells | mmu-miR-23b-3p | MIMAT0000125 | 19582816 |
| Stem Cells | mmu-miR-23b-3p | MIMAT0000125 | 20216554 |
| Stem Cells | mmu-miR-24-3p | MIMAT0000219 | 19582816 |
| Stem Cells | mmu-miR-27b-3p | MIMAT0000126 | 19582816 |
| Stem Cells | mmu-miR-290a-3p | MIMAT0004572 | 19628328 |
| Stem Cells | mmu-miR-291a-3p | MIMAT0000368 | 19628328 |
| Stem Cells | mmu-miR-292-3p | MIMAT0000370 | 19628328 |
| Stem Cells | mmu-miR-293-3p | MIMAT0000371 | 19628328 |
| Stem Cells | mmu-miR-294-3p | MIMAT0000372 | 19628328 |
| Stem Cells | mmu-miR-295-3p | MIMAT0000373 | 19628328 |
| Stem Cells | mmu-miR-296-5p | MIMAT0000374 | 18806776 |
| Stem Cells | mmu-miR-29a-3p | MIMAT0000535 | 21665270 |
| Stem Cells | mmu-miR-29b-3p | MIMAT0000127 | 21665270 |
| Stem Cells | mmu-miR-324-5p | MIMAT0000555 | 20216554 |
| Stem Cells | mmu-miR-326-3p | MIMAT0000559 | 20216554 |
| Stem Cells | mmu-miR-34a-5p | MIMAT0000542 | 20424141 |
| Stem Cells | mmu-miR-466l-3p | MIMAT0005830 | 20410487 |
| Stem Cells | mmu-miR-470-5p | MIMAT0002111 | 18806776 |
| Stem Cells | mmu-miR-9-5p | MIMAT0000142 | 22465325 |
| Stem Cells | mmu-miR-9-5p | MIMAT0000142 | 21238922 |
| Stem Cells | mmu-miR-9-5p | MIMAT0000142 | 20362537 |
| Stem Cells | mmu-miR-93-5p | MIMAT0000540 | 21285944 |
| Stem Cells | ppy-miR-130a | MIMAT0015753 | 17855557 |
| Stem Cells | ppy-miR-335 | MIMAT0015837 | 18185580 |
| Stem Cells | ptr-miR-130a | MIMAT0008025 | 17855557 |
| Stem Cells | ptr-miR-335 | MIMAT0008104 | 18185580 |
| Stem Cells | rno-miR-133b-3p | MIMAT0003126 | 19695767 |
| Stem Cells | rno-miR-31a-5p | MIMAT0000810 | 23270756 |
| Stem Cells | rno-miR-335 | MIMAT0000575 | 18185580 |
| Stem Cells | rno-miR-9a-5p | MIMAT0000781 | 18987208 |
| Stem Cells | ssc-miR-130a | MIMAT0007758 | 17855557 |
| Stem Cells | ssc-miR-335 | MIMAT0013955 | 18185580 |
| Stem Cells | xtr-miR-130a | MIMAT0003591 | 17855557 |
| Sternum | mmu-miR-140-5p | MIMAT0000151 | 16828749 |
| Stomach | hsa-miR-106a-5p | MIMAT0000103 | 16461460 |
| Stomach | hsa-miR-181c-5p | MIMAT0000258 | 20080834 |
| Stomach | hsa-miR-20a-5p | MIMAT0000075 | 16461460 |
| Stomach | hsa-miR-21-5p | MIMAT0000076 | 18372920 |
| Stomach | hsa-miR-26a-5p | MIMAT0000082 | 16461460 |
| Stomach | hsa-miR-34a-5p | MIMAT0000255 | 21240262 |
| Stomach | mmu-miR-21a-5p | MIMAT0000530 | 18372920 |
| Stress Fibers | hsa-miR-124-3p | MIMAT0000422 | 21672940 |
| Stress Fibers | hsa-miR-138-5p | MIMAT0000430 | 20232393 |
| Stress Fibers | hsa-miR-200b-3p | MIMAT0000318 | 22144583 |
| Stress Fibers | hsa-miR-21-5p | MIMAT0000076 | 21347332 |
| Stress Fibers | rno-miR-200c-3p | MIMAT0000873 | 22144583 |
| Stromal Cells | hsa-let-7f-5p | MIMAT0000067 | 22407818 |
| Stromal Cells | hsa-miR-125a-5p | MIMAT0000443 | 19011087 |
| Stromal Cells | hsa-miR-125b-5p | MIMAT0000423 | 19011087 |
| Stromal Cells | hsa-miR-196b-5p | MIMAT0001080 | 23293219 |
| Stromal Cells | hsa-miR-199a-5p | MIMAT0000231 | 19011087 |
| Stromal Cells | hsa-miR-199a-5p | MIMAT0000231 | 22441842 |
| Stromal Cells | hsa-miR-222-3p | MIMAT0000279 | 19589872 |
| Stromal Cells | hsa-miR-26a-5p | MIMAT0000082 | 19011087 |
| Stromal Cells | hsa-miR-346 | MIMAT0000773 | 19011087 |
| Stromal Cells | hsa-miR-369-5p | MIMAT0001621 | 21136442 |
| Stromal Cells | mmu-miR-21a-5p | MIMAT0000530 | 18556655 |
| Stromal Cells | mmu-miR-2861 | MIMAT0013803 | 19920351 |
| Stromal Cells | mmu-miR-3960 | MIMAT0019336 | 21324897 |
| Subcutaneous Fat | hsa-miR-519d-3p | MIMAT0002853 | 20057369 |
| Submandibular Gland | mmu-miR-21a-5p | MIMAT0000530 | 21295561 |
| Suprachiasmatic Nucleus | bta-miR-132 | MIMAT0003812 | 17553428 |
| Suprachiasmatic Nucleus | bta-miR-219 | MIMAT0030444 | 17553428 |
| Suprachiasmatic Nucleus | cfa-miR-132 | MIMAT0006732 | 17553428 |
| Suprachiasmatic Nucleus | dre-miR-219 | MIMAT0001286 | 17553428 |
| Suprachiasmatic Nucleus | mml-miR-219 | MIMAT0002575 | 17553428 |
| Suprachiasmatic Nucleus | mmu-miR-132-3p | MIMAT0000144 | 17553428 |
| Suprachiasmatic Nucleus | mmu-miR-132-3p | MIMAT0000144 | 21118894 |
| Suprachiasmatic Nucleus | mmu-miR-132-5p | MIMAT0016984 | 21118894 |
| Suprachiasmatic Nucleus | mmu-miR-219a-5p | MIMAT0000664 | 17553428 |
| Suprachiasmatic Nucleus | mmu-miR-7b-5p | MIMAT0000678 | 17028171 |
| Suprachiasmatic Nucleus | ppy-miR-132 | MIMAT0015755 | 17553428 |
| Suprachiasmatic Nucleus | ppy-miR-219 | MIMAT0002577 | 17553428 |
| Suprachiasmatic Nucleus | ssc-miR-132 | MIMAT0025361 | 17553428 |
| Suprachiasmatic Nucleus | ssc-miR-219 | MIMAT0020590 | 17553428 |
| Suprachiasmatic Nucleus | xtr-miR-132 | MIMAT0003594 | 17553428 |
| Suprachiasmatic Nucleus | xtr-miR-219 | MIMAT0003632 | 17553428 |
| Sympathetic Nervous | hsa-miR-152-3p | MIMAT0000438 | 20841484 |
| System | | | |
| Sympathetic Nervous | hsa-miR-184 | MIMAT0000454 | 20409325 |
| System | | | |
| Sympathetic Nervous | hsa-miR-18a-5p | MIMAT0000072 | 20080637 |
| System | | | |
| Sympathetic Nervous | hsa-miR-19a-3p | MIMAT0000073 | 20080637 |
| System | | | |
| Sympathetic Nervous | hsa-miR-34a-5p | MIMAT0000255 | 21266077 |
| System | | | |
| Synapses | hsa-miR-137 | MIMAT0000429 | 22003227 |
| Synapses | mmu-miR-206-3p | MIMAT0000239 | 20007902 |
| Synapses | mmu-miR-34a-5p | MIMAT0000542 | 18835850 |
| Synapses | rno-miR-138-5p | MIMAT0000844 | 20064393 |
| Synapses | rno-miR-181a-5p | MIMAT0000858 | 22144581 |
| Synovial Fluid | hsa-miR-146a-5p | MIMAT0000449 | 20459811 |
| T-Lymphocytes | hsa-let-7d-5p | MIMAT0000065 | 21616524 |
| T-Lymphocytes | hsa-let-7f-5p | MIMAT0000067 | 21616524 |
| T-Lymphocytes | hsa-let-7g-5p | MIMAT0000414 | 21616524 |
| T-Lymphocytes | hsa-let-7i-5p | MIMAT0000415 | 21616524 |
| T-Lymphocytes | hsa-miR-106a-5p | MIMAT0000103 | 17575136 |
| T-Lymphocytes | hsa-miR-125a-5p | MIMAT0000443 | 20589685 |
| T-Lymphocytes | hsa-miR-126-3p | MIMAT0000445 | 21165896 |
| T-Lymphocytes | hsa-miR-130b-3p | MIMAT0000691 | 18974142 |
| T-Lymphocytes | hsa-miR-143-3p | MIMAT0000435 | 19464056 |
| T-Lymphocytes | hsa-miR-146a-5p | MIMAT0000449 | 19965651 |
| T-Lymphocytes | hsa-miR-146a-5p | MIMAT0000449 | 20459811 |
| T-Lymphocytes | hsa-miR-148a-3p | MIMAT0000243 | 19435428 |
| T-Lymphocytes | hsa-miR-148a-3p | MIMAT0000243 | 20483747 |
| T-Lymphocytes | hsa-miR-155-5p | MIMAT0000646 | 21310411 |
| T-Lymphocytes | hsa-miR-155-5p | MIMAT0000646 | 20673989 |
| T-Lymphocytes | hsa-miR-155-5p | MIMAT0000646 | 20209161 |
| T-Lymphocytes | hsa-miR-155-5p | MIMAT0000646 | 20680360 |
| T-Lymphocytes | hsa-miR-155-5p | MIMAT0000646 | 21304824 |
| T-Lymphocytes | hsa-miR-155-5p | MIMAT0000646 | 22387553 |
| T-Lymphocytes | hsa-miR-181c-5p | MIMAT0000258 | 21112091 |
| T-Lymphocytes | hsa-miR-184 | MIMAT0000454 | 19286996 |
| T-Lymphocytes | hsa-miR-19b-3p | MIMAT0000074 | 19435428 |
| T-Lymphocytes | hsa-miR-19b-3p | MIMAT0000074 | 17575136 |
| T-Lymphocytes | hsa-miR-20a-5p | MIMAT0000075 | 19435428 |
| T-Lymphocytes | hsa-miR-20b-5p | MIMAT0001413 | 17575136 |
| T-Lymphocytes | hsa-miR-21-5p | MIMAT0000076 | 22387553 |
| T-Lymphocytes | hsa-miR-21-5p | MIMAT0000076 | 21469086 |
| T-Lymphocytes | hsa-miR-21-5p | MIMAT0000076 | 20483747 |
| T-Lymphocytes | hsa-miR-21-5p | MIMAT0000076 | 23416424 |
| T-Lymphocytes | hsa-miR-214-3p | MIMAT0000271 | 19435428 |
| T-Lymphocytes | hsa-miR-214-3p | MIMAT0000271 | 20548023 |
| T-Lymphocytes | hsa-miR-221-3p | MIMAT0000278 | 20110463 |
| T-Lymphocytes | hsa-miR-222-3p | MIMAT0000279 | 23522449 |
| T-Lymphocytes | hsa-miR-26a-5p | MIMAT0000082 | 23338972 |
| T-Lymphocytes | hsa-miR-29a-3p | MIMAT0000086 | 19584290 |
| T-Lymphocytes | hsa-miR-30b-5p | MIMAT0000420 | 22387553 |
| T-Lymphocytes | hsa-miR-31-3p | MIMAT0004504 | 19408243 |
| T-Lymphocytes | hsa-miR-31-5p | MIMAT0000089 | 19408243 |
| T-Lymphocytes | hsa-miR-520h | MIMAT0002867 | 19435428 |
| T-Lymphocytes | hsa-miR-92a-3p | MIMAT0000092 | 17575136 |
| T-Lymphocytes | hsa-miR-92a-3p | MIMAT0000092 | 19435428 |
| T-Lymphocytes | hsa-miR-93-5p | MIMAT0000093 | 18974142 |
| T-Lymphocytes | hsa-miR-98-5p | MIMAT0000096 | 20589685 |
| T-Lymphocytes | hsa-miR-98-5p | MIMAT0000096 | 21616524 |
| T-Lymphocytes | mmu-miR-106a-5p | MIMAT0000385 | 17575136 |
| T-Lymphocytes | mmu-miR-124-3p | MIMAT0000134 | 21131957 |
| T-Lymphocytes | mmu-miR-125b-5p | MIMAT0000136 | 21118986 |
| T-Lymphocytes | mmu-miR-133a-3p | MIMAT0000145 | 20173049 |
| T-Lymphocytes | mmu-miR-142-3p | MIMAT0000155 | 19098714 |
| T-Lymphocytes | mmu-miR-155-5p | MIMAT0000165 | 19144316 |
| T-Lymphocytes | mmu-miR-155-5p | MIMAT0000165 | 17463290 |
| T-Lymphocytes | mmu-miR-155-5p | MIMAT0000165 | 21149603 |
| T-Lymphocytes | mmu-miR-181a-5p | MIMAT0000210 | 17344418 |
| T-Lymphocytes | mmu-miR-19b-3p | MIMAT0000513 | 17575136 |
| T-Lymphocytes | mmu-miR-20b-5p | MIMAT0003187 | 17575136 |
| T-Lymphocytes | mmu-miR-214-3p | MIMAT0000661 | 20548023 |
| T-Lymphocytes | mmu-miR-92a-3p | MIMAT0000539 | 17575136 |
| T-Lymphocytes | rno-miR-23a-3p | MIMAT0000792 | 19574461 |
| T-Lymphocytes | rno-miR-27a-3p | MIMAT0000799 | 19574461 |
| T-Lymphocytes Helper- | hsa-miR-21-5p | MIMAT0000076 | 23416424 |
| Inducer | | | |
| T-Lymphocytes Helper- | mmu-miR-326-3p | MIMAT0000559 | 19838199 |
| Inducer | | | |
| T-Lymphocytes Regulatory | hsa-let-7i-5p | MIMAT0000415 | 21742974 |
| T-Lymphocytes Regulatory | hsa-miR-155-5p | MIMAT0000646 | 20209161 |
| T-Lymphocytes Regulatory | hsa-miR-155-5p | MIMAT0000646 | 21304824 |
| T-Lymphocytes Regulatory | hsa-miR-21-5p | MIMAT0000076 | 23416424 |
| T-Lymphocytes Regulatory | mmu-miR-142-3p | MIMAT0000155 | 19098714 |
| T-Lymphocytes Regulatory | mmu-miR-146a-5p | MIMAT0000158 | 20850013 |
| T-Lymphocytes Regulatory | mmu-miR-155-5p | MIMAT0000165 | 19144316 |
| T-Lymphocytes Regulatory | mmu-miR-155-5p | MIMAT0000165 | 21149603 |
| Telencephalon | mmu-miR-9-5p | MIMAT0000142 | 18842901 |
| Testis | hsa-miR-383-5p | MIMAT0000738 | 21368870 |
| Testis | mmu-miR-709 | MIMAT0003499 | 19029807 |
| Thymus Gland | hsa-miR-433-3p | MIMAT0001627 | 20181727 |
| Thymus Gland | hsa-miR-7-5p | MIMAT0000252 | 18823940 |
| Thymus Gland | mmu-miR-29a-3p | MIMAT0000535 | 19959559 |
| Thymus Gland | mmu-miR-29b-3p | MIMAT0000127 | 19959559 |
| Thymus Gland | mmu-miR-468-3p | MIMAT0002109 | 19959559 |
| Thyroid Gland | hsa-let-7f-5p | MIMAT0000067 | 19956384 |
| Thyroid Gland | hsa-miR-125b-5p | MIMAT0000423 | 17563749 |
| Thyroid Gland | hsa-miR-138-5p | MIMAT0000430 | 18201269 |
| Thyroid Gland | hsa-miR-146a-5p | MIMAT0000449 | 20061417 |
| Thyroid Gland | hsa-miR-146b-5p | MIMAT0002809 | 16365291 |
| Thyroid Gland | hsa-miR-192-5p | MIMAT0000222 | 16822819 |
| Thyroid Gland | hsa-miR-197-3p | MIMAT0000227 | 16822819 |
| Thyroid Gland | hsa-miR-204-5p | MIMAT0000265 | 20691260 |
| Thyroid Gland | hsa-miR-206 | MIMAT0000462 | 20388878 |
| Thyroid Gland | hsa-miR-221-3p | MIMAT0000278 | 20388878 |
| Thyroid Gland | hsa-miR-221-3p | MIMAT0000278 | 16365291 |
| Thyroid Gland | hsa-miR-222-3p | MIMAT0000279 | 20388878 |
| Thyroid Gland | hsa-miR-222-3p | MIMAT0000279 | 16365291 |
| Thyroid Gland | hsa-miR-26a-5p | MIMAT0000082 | 17563749 |
| Thyroid Gland | hsa-miR-27a-3p | MIMAT0000084 | 21149577 |
| Thyroid Gland | hsa-miR-320a | MIMAT0000510 | 19164563 |
| Thyroid Gland | hsa-miR-346 | MIMAT0000773 | 16822819 |
| Thyroid Gland | rno-miR-27a-3p | MIMAT0000799 | 21149577 |
| Thyroid Gland | rno-miR-34a-5p | MIMAT0000815 | 18704095 |
| Tight Junctions | hsa-miR-1 | MIMAT0000416 | 23142026 |
| Tight Junctions | hsa-miR-155-5p | MIMAT0000646 | 18794355 |
| Tight Junctions | hsa-miR-203a | MIMAT0000264 | 22101077 |
| Tight Junctions | hsa-miR-212-3p | MIMAT0000269 | 18162065 |
| Tight Junctions | hsa-miR-483-3p | MIMAT0002173 | 22101077 |
| Tight Junctions | hsa-miR-595 | MIMAT0003263 | 22101077 |
| Tight Junctions | mmu-miR-155-5p | MIMAT0000165 | 18794355 |
| Tongue | hsa-miR-133a-3p | MIMAT0000427 | 18464261 |
| Tongue | hsa-miR-133b | MIMAT0000770 | 18464261 |
| Tongue | hsa-miR-138-5p | MIMAT0000430 | 20232393 |
| Tongue | hsa-miR-138-5p | MIMAT0000430 | 20819078 |
| Tongue | hsa-miR-222-3p | MIMAT0000279 | 19487542 |
| Tongue | hsa-miR-24-3p | MIMAT0000080 | 20816961 |
| Tongue | hsa-miR-7-5p | MIMAT0000252 | 20819078 |
| Trabecular Meshwork | hsa-miR-106a-5p | MIMAT0000103 | 19782699 |
| Trabecular Meshwork | hsa-miR-182-5p | MIMAT0000259 | 19782699 |
| Trabecular Meshwork | hsa-miR-183-5p | MIMAT0000261 | 19940135 |
| Trabecular Meshwork | hsa-miR-24-3p | MIMAT0000080 | 20945401 |
| Trabecular Meshwork | hsa-miR-29b-3p | MIMAT0000100 | 19956414 |
| Trachea | mmu-miR-125b-5p | MIMAT0000136 | 21945074 |
| Trachea | mmu-miR-30a-5p | MIMAT0000128 | 21945074 |
| Trachea | mmu-miR-30c-5p | MIMAT0000514 | 21945074 |
| Trophoblasts | hsa-miR-106a-5p | MIMAT0000103 | 23438603 |
| Trophoblasts | hsa-miR-199b-5p | MIMAT0000263 | 19900756 |
| Trophoblasts | hsa-miR-19b-3p | MIMAT0000074 | 23438603 |
| Trophoblasts | hsa-miR-205-5p | MIMAT0000266 | 20065103 |
| Trophoblasts | hsa-miR-224-5p | MIMAT0000281 | 20065103 |
| Trophoblasts | hsa-miR-335-5p | MIMAT0000765 | 20065103 |
| Trophoblasts | hsa-miR-34a-5p | MIMAT0000255 | 20351093 |
| Trophoblasts | hsa-miR-424-5p | MIMAT0001341 | 20065103 |
| Trophoblasts | hsa-miR-491-5p | MIMAT0002807 | 20065103 |
| Trophoblasts | hsa-miR-93-5p | MIMAT0000093 | 20065103 |
| Umbilical Cord | hsa-miR-129-5p | MIMAT0000242 | 18189265 |
| Umbilical Cord | hsa-miR-135b-5p | MIMAT0000758 | 19795981 |
| Umbilical Cord | hsa-miR-148a-3p | MIMAT0000243 | 19435428 |
| Umbilical Cord | hsa-miR-184 | MIMAT0000454 | 19286996 |
| Umbilical Cord | hsa-miR-19b-3p | MIMAT0000074 | 19435428 |
| Umbilical Cord | hsa-miR-20a-5p | MIMAT0000075 | 19435428 |
| Umbilical Cord | hsa-miR-214-3p | MIMAT0000271 | 19435428 |
| Umbilical Cord | hsa-miR-34a-5p | MIMAT0000255 | 20627091 |
| Umbilical Cord | hsa-miR-520h | MIMAT0002867 | 19435428 |
| Umbilical Cord | hsa-miR-520h | MIMAT0002867 | 18189265 |
| Umbilical Cord | hsa-miR-92a-3p | MIMAT0000092 | 19435428 |
| Umbilical Veins | hsa-miR-155-5p | MIMAT0000646 | 21310411 |
| Umbilical Veins | hsa-miR-155-5p | MIMAT0000646 | 23108656 |
| Umbilical Veins | hsa-miR-19a-3p | MIMAT0000073 | 20133739 |
| Umbilical Veins | hsa-miR-216a-5p | MIMAT0000273 | 19786632 |
| Umbilical Veins | hsa-miR-217 | MIMAT0000274 | 19786632 |
| Umbilical Veins | hsa-miR-409-3p | MIMAT0001639 | 22531314 |
| Umbilical Veins | hsa-miR-519c-3p | MIMAT0002832 | 20233879 |
| Urinary Bladder | hsa-miR-100-5p | MIMAT0000098 | 19843843 |
| Urinary Bladder | hsa-miR-101-3p | MIMAT0000099 | 19258506 |
| Urinary Bladder | hsa-miR-125b-5p | MIMAT0000423 | 20549700 |
| Urinary Bladder | hsa-miR-125b-5p | MIMAT0000423 | 19378336 |
| Urinary Bladder | hsa-miR-129-5p | MIMAT0000242 | 19487295 |
| Urinary Bladder | hsa-miR-133a-3p | MIMAT0000427 | 19378336 |
| Urinary Bladder | hsa-miR-133a-3p | MIMAT0000427 | 20160723 |
| Urinary Bladder | hsa-miR-143-3p | MIMAT0000435 | 23104321 |
| Urinary Bladder | hsa-miR-143-3p | MIMAT0000435 | 19157460 |
| Urinary Bladder | hsa-miR-145-5p | MIMAT0000437 | 21360565 |
| Urinary Bladder | hsa-miR-145-5p | MIMAT0000437 | 23104321 |
| Urinary Bladder | hsa-miR-145-5p | MIMAT0000437 | 19915607 |
| Urinary Bladder | hsa-miR-145-5p | MIMAT0000437 | 19378336 |
| Urinary Bladder | hsa-miR-145-5p | MIMAT0000437 | 20160723 |
| Urinary Bladder | hsa-miR-195-5p | MIMAT0000461 | 22265971 |
| Urinary Bladder | hsa-miR-195-5p | MIMAT0000461 | 19378336 |
| Urinary Bladder | hsa-miR-199a-3p | MIMAT0000232 | 21807947 |
| Urinary Bladder | hsa-miR-199a-3p | MIMAT0000232 | 19378336 |
| Urinary Bladder | hsa-miR-200b-3p | MIMAT0000318 | 19671845 |
| Urinary Bladder | hsa-miR-200c-3p | MIMAT0000617 | 19671845 |
| Urinary Bladder | hsa-miR-203a | MIMAT0000264 | 21205209 |
| Urinary Bladder | hsa-miR-21-5p | MIMAT0000076 | 21468550 |
| Urinary Bladder | hsa-miR-30a-3p | MIMAT0000088 | 19378336 |
| Urinary Bladder | hsa-miR-30a-5p | MIMAT0000087 | 19378336 |
| Urinary Bladder | hsa-miR-34a-5p | MIMAT0000255 | 21240262 |
| Urinary Bladder | hsa-miR-429 | MIMAT0001536 | 19671845 |
| Urinary Bladder | hsa-miR-99a-5p | MIMAT0000097 | 19843843 |
| Urothelium | hsa-miR-100-5p | MIMAT0000098 | 19843843 |
| Urothelium | hsa-miR-125b-5p | MIMAT0000423 | 20549700 |
| Urothelium | hsa-miR-129-5p | MIMAT0000242 | 19487295 |
| Urothelium | hsa-miR-145-5p | MIMAT0000437 | 19915607 |
| Urothelium | hsa-miR-99a-5p | MIMAT0000097 | 19843843 |
| Uterus | mmu-miR-101a-3p | MIMAT0000133 | 17848513 |
| Uterus | mmu-miR-199a-3p | MIMAT0000230 | 17848513 |
| Uterus | mmu-miR-21a-5p | MIMAT0000530 | 18556655 |
| Veins | hsa-miR-140-5p | MIMAT0000431 | 23401231 |
| Veins | hsa-miR-146a-5p | MIMAT0000449 | 22711166 |
| Veins | hsa-miR-155-5p | MIMAT0000646 | 23108656 |
| Veins | hsa-miR-16-5p | MIMAT0000069 | 19738602 |
| Veins | hsa-miR-328-3p | MIMAT0000752 | 18560585 |
| Veins | hsa-miR-328-5p | MIMAT0026486 | 18560585 |
| Veins | hsa-miR-409-3p | MIMAT0001639 | 22531314 |
| X Chromosome | hsa-miR-106a-5p | MIMAT0000103 | 17575136 |
| X Chromosome | hsa-miR-19b-3p | MIMAT0000074 | 17575136 |
| X Chromosome | hsa-miR-20b-5p | MIMAT0001413 | 17575136 |
| X Chromosome | hsa-miR-221-3p | MIMAT0000278 | 17721077 |
| X Chromosome | hsa-miR-222-3p | MIMAT0000279 | 17721077 |
| X Chromosome | hsa-miR-92a-3p | MIMAT0000092 | 17575136 |
| X Chromosome | mmu-miR-106a-5p | MIMAT0000385 | 17575136 |
| X Chromosome | mmu-miR-19b-3p | MIMAT0000513 | 17575136 |
| X Chromosome | mmu-miR-20b-5p | MIMAT0003187 | 17575136 |
| X Chromosome | mmu-miR-92a-3p | MIMAT0000539 | 17575136 |

TABLE I
IncRNA-chromatin interaction data - H. sapiens, taken from Supp. Table 1 of
Zhang et al, JBC 2019, doi: 10.1074/jbc.RA119.008732; jbc.RA119.008732.
LncRNA-chromatin interaction data in human

| | | | | | IncRNA | |
|---|---|---|---|---|---|---|
| | | | | | control | |
| | | Detected | Cell Type | IncRNA IP | sample | |
| IncRNA | Method | Cell type | Description | sample ID | ID | PMID |
| HOTAIR | ChlRP- | MDAMB2 | Breast | GSM820433, | GSM820435 | 21963238 |
| | Seq | 31 | Cancer | GSM820434 | | |
| LED | ChlRP- | MCF7 | Breast | GSM1294974, | GSM1294973 | 25813522 |
| | Seq | with | Cancer | GSM1294975 | | |
| | | nutlin-3a | | | | |
| MEG3 | ChOP- | BT-549 | Breast | PRJEB7307 | | 26205790 |
| | seq | | Cancer | | | |
| MALAT1 | CHART- | MCF7 | Breast | GSM1411209, | GSM1411212 | 25155612 |
| | seq | | Cancer | GSM1411210 | | |
| NEAT1 | CHART- | MCF7 | Breast | GSM1411207, | GSM1411211 | 25155612 |
| | seq | | Cancer | GSM1411208 | | |
| 7SK | ChlRP- | HeLaS3 | Cervical | GSM1693801, | GSM1693803 | 26878240 |
| | Seq | | cancer | GSM1693802 | | |
| TERC | ChlRP- | HeLaS3 | Cervical | GSM820437, | GSM820432 | 21963238 |
| | Seq | | cancer | GSM820438 | | |
| *DACOR1 | ChlRP- | V852 | Colon | GSM1854427 | GSM1854426 | 26307088 |
| | Seq | | cancer | (1 run) | | |
| SNHG1 | ChlRP- | HCT116 | Colon | GSM2285442, | GSM2285451, | 28825722 |
| | Seq | | cancer | GSM2285443, | GSM2285452, | |
| | | | | GSM2285444, | GSM2285453 | |
| | | | | GSM2285445, | | |
| | | | | GSM2285446, | | |
| | | | | GSM2285447 | | |
| DINO | ChlRP- | U2OS | Human | GSM2011201, | GSM2011205 | 27668660 |
| | Seq | | Bone | GSM2011202 | | |
| | | | Osteosarcoma | | | |
| | | | Epithelial | | | |
| | | | Cells | | | |
| 7SK | ChlRP- | H1 | Embryonic | GSM1693798, | GSM1693800 | 26878240 |
| | Seq | | stem cell | GSM1693799 | | |
| SRA | ChlRP- | NTERA2 | Pluripotent | GSM1415922 | GSM1415924 | 26496121 |
| | Seq | | human | (3 runs) | | |
| | | | embryonal | | | |
| | | | carcinoma | | | |
| | | | cell line | | | |
| *Long | ChlRP- | Erythroblast | Blood | GSM2449980, | GSM2449979 | 28132025 |
| noncoding | Seq | | | GSM2449981 | | |
| RNAs | | | | | | |
| transcribed | | | | | | |
| by | | | | | | |
| ERV-9 | | | | | | |
| LTR retro- | | | | | | |
| transposon | | | | | | |

TABLE J
De novo lncRNA binding motifs discovered
in human lncRNA-chromatin interaction
data - H. sapiens, taken from Supp. Table
2 of Zhang et al, JBC 2019 doi: 10.1074/
jbc.RA119.008732; jbc.RA119.008732.

| | | | # of | |
|---|---|---|---|---|
| | | | Matched | SEQ |
| | denovo | | motif | ID |
| | motif | | instance | NO: |
| lncRNA | (Homer) | p−value | (Fimo) | (#) |
| HOTAIR. | GGCATTTCTGGT | 1.00E−123 | 3 | 481 |
| MDAMB231 | | | | |
| | CAAGACCAGAGA | 1.00E−101 | 5 | 482 |
| | ATGGGAGGCGCA | 1.00E−93 | 1 | 483 |
| | GCAGAGAAAAGG | 1.00E−90 | 14 | 484 |
| | GTTTGGGCCTCC | 1.00E−87 | 6 | 485 |
| | CCTGTATGGAAC | 1.00E−81 | 1 | 486 |
| | GTGTCTACATGC | 1.00E−71 | 2 | 487 |
| | CAGGAGGAAGTT | 1.00E−67 | 1 | 488 |
| | AGAAAAGGCTGA | 1.00E−65 | 10 | 489 |
| | TGGAACAAGCAT | 1.00E−64 | 2 | 490 |
| | AGGAGGAAGTTC | 1.00E−58 | 5 | 491 |
| | AGAGCAAGGAAG | 1.00E−54 | 9 | 492 |
| | GGAGGGGCGTCT | 1.00E−51 | 4 | 493 |
| | GGCCCCAAAGAG | 1.00E−45 | 8 | 494 |
| | AGGAGGCCCAAA | 1.00E−42 | 3 | 495 |
| | TTAGCGCCTCCC | 1.00E−42 | 1 | 496 |
| | GGGCTGGTTTCA | 1.00E−40 | 5 | 497 |
| | TAACTGGCAGCA | 1.00E−38 | 2 | 498 |
| | ACATGCATCACT | 1.00E−35 | 1 | 499 |
| | KGACTTWGCACT | 1.00E−32 | 1 | 500 |
| | GTGCTGATTCCT | 1.00E−27 | 1 | 501 |
| | CACTGCCAACCC | 1.00E−25 | 7 | 502 |
| | GGGACAAAAGTT | 1.00E−23 | 4 | 503 |
| | TTTCTGACACTG | 1.00E−23 | 1 | 504 |
| | CAGAGAAAGGCT | 1.00E−15 | 5 | 505 |
| MEG3. | GAGAGAGAGAGA | 1.00E−35 | 76 | 506 |
| BT549 | | | | |
| | GTGTGTGTGTGT | 1.00E−23 | 97 | 507 |
| | TGTATGTGTGTG | 1.00E−19 | 77 | 508 |
| | CAYWGTGTGTGT | 1.00E−12 | 55 | 509 |
| | CGGGGTCGBAGC | 1.00E−11 | 8 | 510 |
| | GTTTTTCTAGAA | 1.00E−09 | 9 | 511 |
| | ATCATATCAAAA | 1.00E−08 | 5 | 512 |
| | AGAACAGATGTA | 1.00E−08 | 1 | 513 |
| | TGTGTTTCTTCA | 1.00E−07 | 12 | 514 |
| | GGTTTTTCACCT | 1.00E−07 | 7 | 515 |
| | ATAGAAAGAAAT | 1.00E−07 | 21 | 516 |
| | TSTMTCTGTMTG | 1.00E−07 | 20 | 517 |
| | GMTCMTATCTAT | 1.00E−07 | 3 | 518 |
| | GGGGGGGGGGGG | 1.00E−04 | 72 | 519 |
| | TATATATATATA | 1.00E−04 | 9 | 520 |
| | TCTCTCTCTTTT | 1.00E−02 | 45 | 521 |
| | TCTTTCCTTCCT | 1.00E−01 | 54 | 522 |
| 7SK.H1 | KGGCTGGCTGGC | 1e−625 | 3 | 523 |
| | ATCTGTCACCCC | 1.00E−271 | 1 | 524 |
| | GACTCCAGACAC | 1.00E−249 | 1 | 525 |
| | TCCAGACACATC | 1.00E−221 | 1 | 526 |
| | CACATGGAGCNG | 1.00E−184 | 2 | 527 |
| | AGACTCCAGACM | 1.00E−170 | 1 | 528 |
| | GCCCTCACATCC | 1.00E−159 | NA | 529 |
| | GGGACGCACATG | 1.00E−153 | 1 | 530 |
| | BCCAGATCAGCC | 1.00E−152 | 2 | 531 |
| | CCTAGCCAGCCR | 1.00E−149 | 1 | 532 |
| | NCAARACTHCAS | 1.00E−147 | 1 | 533 |
| | CCCTACGTTCTC | 1.00E−130 | 1 | 534 |
| | TGTCACCCCATT | 1.00E−130 | 1 | 535 |
| | TGGACCTTGAGA | 1.00E−98 | 1 | 536 |
| | TACAATGGACCT | 1.00E−89 | 1 | 537 |
| | AGCTCTCAAGGT | 1.00E−75 | 1 | 538 |
| | ARCGTAGGGTAG | 1.00E−68 | 1 | 539 |
| | SATGGAGCGGTG | 1.00E−51 | 1 | 540 |
| | AAGAGGACGACC | 1.00E−41 | 2 | 541 |
| | TGACTACCCTAC | 1.00E−38 | 1 | 542 |
| | GGACCTTGGAGC | 1.00E−37 | NA | 543 |
| | GCACTGGAGCGG | 1.00E−33 | NA | 544 |
| | TGGCTGGYGTAG | 1.00E−33 | NA | 545 |
| | GGGCCACCATCA | 1.00E−28 | NA | 546 |
| | TTGTGCAGCCTC | 1.00E−24 | NA | 547 |
| 7SK. | GCCAGCCAGMYM | 1e−402 | 3 | 548 |
| HeLaS3 | | | | |
| | GCCCTCACATCC | 1.00E−240 | NA | 529 |
| | TGGCTGGCTAGG | 1.00E−227 | 1 | 549 |
| | CCCTACGTTCTC | 1.00E−226 | 1 | 534 |
| | GACTCCAGACAC | 1.00E−218 | 1 | 525 |
| | TGTCTGGAGTCT | 1.00E−137 | 1 | 550 |
| | CCAGCCAGCTCM | 1.00E−120 | 1 | 551 |
| | CCAAGACTCCAG | 1.00E−120 | 1 | 552 |
| | GGGGTGACAGAT | 1.00E−111 | 1 | 553 |
| | TACCCTACGTTC | 1.00E−106 | 1 | 554 |
| | TGTCACCCCATT | 1.00E−105 | 1 | 535 |
| | GCCAGATCAGCC | 1.00E−98 | 2 | 555 |
| | GACTACCCTACG | 1.00E−78 | 1 | 556 |
| | CACATGGAGCSS | 1.00E−68 | 2 | 557 |
| | GCTCTCAAGGTC | 1.00E−67 | 1 | 558 |
| | GACGCACATGGA | 1.00E−66 | 1 | 559 |
| | CATGTGCGTCCC | 1.00E−65 | 1 | 560 |
| | CTACGTTTCTCC | 1.00E−64 | NA | 561 |
| | AGGGTAGTCAAG | 1.00E−56 | 1 | 562 |
| | GSCGCCATCTTG | 1.00E−45 | NA | 563 |
| | CAAGGTCCATTG | 1.00E−33 | 1 | 564 |
| | TCGCCAGGGTTG | 1.00E−33 | 1 | 565 |
| | GGTCGTCCTCTT | 1.00E−32 | 2 | 566 |
| | GGAAGGGGACAC | 1.00E−18 | 1 | 567 |
| | ACGCAATTACTC | 1.00E−18 | 1 | 568 |
| SNHG1. | GGCCTGGATCAT | 1.00E−75 | 18 | 569 |
| HCT116 | | | | |
| | GAGAGCTCTGTG | 1.00E−50 | 29 | 570 |
| | CCTGGATCATGT | 1.00E−50 | 8 | 571 |
| | GTGGAGTTATGG | 1.00E−41 | 6 | 572 |
| | CGCTGGCTTTGC | 1.00E−35 | 22 | 573 |
| | GCTCTTGTGGSC | 1.00E−35 | 5 | 574 |
| | TTCCCATAACTC | 1.00E−33 | 8 | 575 |
| | ATACAGCCACCG | 1.00E−30 | 14 | 576 |
| | GCAGACACAGAT | 1.00E−30 | 8 | 577 |
| | AGAGCAGCTTGT | 1.00E−26 | 7 | 578 |
| | AACTCAGGCACT | 1.00E−25 | 13 | 579 |
| | AGCCCACAGAGC | 1.00E−23 | 9 | 580 |
| | TTGGGAAGTTCA | 1.00E−23 | 27 | 581 |
| | ACTGCTGTTTCA | 1.00E−19 | 12 | 582 |
| | CTACCTACATGG | 1.00E−16 | 12 | 583 |
| | GCTAATGATAGC | 1.00E−13 | NA | 584 |
| | TGCRARCCAGCC | 1.00E−09 | 11 | 585 |
| | GGGGACAGCCAC | 1.00E−09 | 6 | 586 |
| | KACCTYCCCCAA | 1.00E−07 | NA | 587 |
| | GVTCCTGGRTCS | 1.00E−07 | 8 | 588 |
| | CTGGTCATTGGT | 1.00E−06 | 4 | 589 |
| | GCTGGTCTGAGT | 1.00E−05 | 2 | 590 |
| | TATCAGAGGTCA | 1.00E−05 | 4 | 591 |
| | GAGATATGGGCC | 1.00E−04 | 8 | 592 |
| | CCTATCTACGCC | 1.00E−04 | NA | 593 |
| MALAT1. | GGTTTGCCGCWG | 1.00E−09 | NA | 594 |
| MCF7 | | | | |
| | CTCAGCTGCCAC | 1.00E−05 | 22 | 595 |
| | GGAGCTCACGGG | 1.00E−05 | 2 | 596 |
| | ATATTCTACCGG | 1.00E−02 | NA | 597 |
| | GTCTTTACGACA | 1.00E−02 | 4 | 598 |
| | GGAATATTCTAC | 1.00E−02 | 4 | 599 |
| | TTTATGACACCG | 1.00E−02 | 5 | 600 |
| | CGGTGGAGGAAT | 1.00E−02 | 3 | 601 |
| | GAGTCTGTCCCG | 1.00E−02 | 3 | 602 |
| NEAT1- | YCYYCYCCCYYY | 1.00E−71 | 74 | 603 |
| MCF7 | | | | |
| | SCGATTGGYYGR | 1.00E−59 | 3 | 604 |
| | AAGGGGGTGGAG | 1.00E−50 | 29 | 605 |
| | GGRRGAAARRAA | 1.00E−45 | 46 | 606 |
| | TVGCCCCGCCCC | 1.00E−44 | 14 | 607 |
| | CASCAGRGGGCA | 1.00E−42 | 12 | 608 |
| | CCCTCCCCCAAC | 1.00E−19 | 28 | 609 |
| | VTGCGCATGCGC | 1.00E−19 | 2 | 610 |
| | ATTGGCAGGCGG | 1.00E−17 | 10 | 611 |
| | CGGTCACGTGAC | 1.00E−11 | 5 | 612 |
| | CACCACCTAGTG | 1.00E−11 | 8 | 613 |
| | CTACAAATCCCA | 1.00E−11 | 8 | 614 |
| | CGGAAGTGACGT | 1.00E−10 | 5 | 615 |
| | TTTGTTTGATCT | 1.00E−10 | 12 | 616 |
| | ACAGTACAAACT | 1.00E−09 | 6 | 617 |
| | AATCTCGCGAGA | 1.00E−09 | NA | 222 |
| | CCGTGACGTCAC | 1.00E−09 | 6 | 618 |
| | CCACCAGGGGTC | 1.00E−08 | 5 | 619 |
| | AGGGTTGTGGCC | 1.00E−07 | 5 | 620 |
| | CGCAACCTAACC | 1.00E−07 | 5 | 621 |
| | GCCCGAAGCTCC | 1.00E−05 | 6 | 622 |
| | CAGACGGCCTTA | 1.00E−05 | 4 | 623 |
| | CTTATTCTCACT | 1.00E−05 | 4 | 624 |
| | TAAAGAAGAAGG | 1.00E−05 | 5 | 625 |
| | TGTGTGTGTGCG | 1.00E−04 | 72 | 626 |
| TERC. | GGGGGCTGGGCA | 1e−1348 | 4 | 627 |
| HeLaS3 | | | | |
| | GGCCACCACCCC | 1e−1177 | 1 | 628 |
| | GGTGGTGGCCAT | 1e−818 | 2 | 629 |
| | GCCTGCCCAGCC | 1e−529 | 1 | 630 |
| | GRGGGCGAGGGS | 1e−439 | 4 | 631 |
| | AAAATGGCCACC | 1e−352 | 2 | 632 |
| | MGGAGGGTGGGC | 1e−325 | 2 | 633 |
| | CCACCACCCCTC | 1.00E−275 | 2 | 634 |
| | GCCACCACCCTC | 1.00E−126 | 1 | 635 |
| | GGCCCACCTCCT | 1.00E−109 | 3 | 636 |
| | GGGGGCTGGCAG | 1.00E−96 | 4 | 637 |
| | GCCTGCCCCAGC | 1.00E−80 | 3 | 638 |
| | CCGCGGCTTCCA | 1.00E−79 | 3 | 639 |
| | GCCTCGTCCTGC | 1.00E−77 | 3 | 640 |
| | STCCCCGCGCGY | 1.00E−72 | 6 | 641 |
| | CCCTCNSCCCTC | 1.00E−69 | 4 | 642 |
| | CTGAGCTGTGGG | 1.00E−58 | 1 | 643 |
| | GTCAGCCGCGGG | 1.00E−55 | 2 | 644 |
| | AGGCCTCGGCTB | 1.00E−53 | 4 | 645 |
| | ACCCAGGACTCG | 1.00E−42 | 2 | 646 |
| | AGGACCACCTCT | 1.00E−39 | NA | 647 |
| | CAGCAGCTGACA | 1.00E−38 | 2 | 648 |
| | GGCTTCKCCAGG | 1.00E−33 | 1 | 649 |
| | TCTKGGCTTTKT | 1.00E−32 | NA | 650 |
| | TCAGTTGTAGAG | 1.00E−24 | NA | 651 |
| LED.MCF7 | CCAACTGGAGGC | 1.00E−249 | 3 | 652 |
| | AGTGAATCACAC | 1.00E−97 | 3 | 653 |
| | RATMMCAACTGG | 1.00E−86 | 1 | 654 |
| | TGAATCACACAC | 1.00E−78 | 5 | 655 |
| | GACACCAGGGTT | 1.00E−71 | 7 | 656 |
| | GGCCAAAGAGGN | 1.00E−64 | 5 | 657 |
| | GGTCTGAGTTCC | 1.00E−56 | 5 | 658 |
| | GGGCTCCAGAAA | 1.00E−55 | 3 | 659 |
| | TTCAAACCCTGG | 1.00E−55 | 3 | 660 |
| | GAGCCCCAAGAA | 1.00E−54 | 6 | 661 |
| | CAAGAACAGCTC | 1.00E−48 | 3 | 662 |
| | HGTGTTTCCTGT | 1.00E−38 | 4 | 663 |
| | AGAAGACGTGAC | 1.00E−35 | 4 | 664 |
| | CCAGAAGAAATC | 1.00E−33 | 3 | 665 |
| | GGGTCACGGAAG | 1.00E−31 | 5 | 666 |
| | AACAGAGCCCCA | 1.00E−31 | 4 | 667 |
| | CAAAGAGCTTTC | 1.00E−31 | 4 | 668 |
| | GGAAGGTCTGAG | 1.00E−31 | 7 | 669 |
| | TTCCCTGTGACC | 1.00E−30 | 6 | 670 |
| | GATCTTCCCTGT | 1.00E−29 | 6 | 671 |
| | AGCTTCATTCAG | 1.00E−27 | 1 | 672 |
| | NAGAGAGCTGTH | 1.00E−26 | 3 | 673 |
| | ACTGAGACTCCT | 1.00E−26 | 3 | 674 |
| | CGCCTAGAAAAC | 1.00E−25 | 1 | 675 |
| | GGTCAAGTCGGT | 1.00E−24 | 3 | 676 |
| SRA. | GCCCCCTAGTGG | 1.00E−80 | NA | 677 |
| NTERA2 | | | | |
| | TTGTTATGCAAA | 1.00E−21 | 1 | 678 |
| | CAAATTACTGCA | 1.00E−16 | 1 | 679 |
| | TTAAAGTGGGCT | 1.00E−11 | 1 | 680 |
| | AGGGAGGGGATG | 1.00E−11 | 2 | 681 |
| | GAGCCTAATAAA | 1.00E−11 | NA | 682 |
| | AGGTTATCTGGT | 1.00E−10 | 1 | 683 |
| | GTTTTCTTTGTG | 1.00E−10 | 4 | 684 |
| | CCAAGATTAATT | 1.00E−09 | 4 | 685 |
| | GAAAAAACCGTT | 1.00E−09 | NA | 686 |
| | CCACSACTAAGC | 1.00E−09 | 3 | 687 |
| | WACYAAATGCGT | 1.00E−09 | NA | 688 |
| | AACATGTCCCTG | 1.00E−08 | NA | 689 |
| | AAATTATTTTAG | 1.00E−07 | NA | 690 |
| | BGCATCTAAGCC | 1.00E−07 | NA | 691 |
| | CTAATGCAGAGA | 1.00E−07 | 2 | 692 |
| | TTGCCTGGAAGG | 1.00E−06 | NA | 693 |
| | ACCCATCCGCCC | 1.00E−06 | 4 | 694 |
| | AGAGTAGACGGT | 1.00E−05 | 1 | 695 |
| | AAGGGTCCAGCC | 1.00E−05 | 7 | 696 |
| | CCACTTGVGATT | 1.00E−05 | 3 | 697 |
| | CAGCCTGTGTAA | 1.00E−05 | 2 | 698 |
| | TTCTAATTATCG | 1.00E−04 | NA | 699 |
| | ACCGACACGGGG | 1.00E−04 | NA | 700 |
| | CGGCGGGARTCT | 1.00E−04 | 5 | 701 |
| DINO. | ATTGCAGAGAGG | 1.00E−116 | untreated | 702 |
| U2OS | | | | |
| | GCAGAGAGGTGC | 1.00E−80 | | 703 |
| | CCCTACACTCAC | 1.00E−60 | | 704 |
| | GAGAATGAGTTG | 1.00E−56 | | 705 |
| | HGGAAATTGCAG | 1.00E−54 | | 706 |
| | VACMCTCACCTG | 1.00E−48 | | 707 |
| | AGAAATCCCTGT | 1.00E−40 | | 708 |
| | GGAGTATTCAGG | 1.00E−30 | | 709 |
| | AACCACAGGGAT | 1.00E−25 | | 710 |
| | TTGGAGAATGAG | 1.00E−22 | | 711 |
| | CACATTTCCCCA | 1.00E−18 | | 712 |
| | TACACCCTACAC | 1.00E−17 | | 713 |
| | GAGATGAGTTGG | 1.00E−17 | | 714 |
| | TCCCTCACTAGG | 1.00E−16 | | 715 |
| | GAAAAACAWAGA | 1.00E−11 | | 716 |
| | CCTGCACTBWCA | 1.00E−11 | | 717 |
| | TCRCYSGGGCAC | 1.00E−11 | | 718 |
| | GCCCTCACGTGG | 1.00E−09 | | 719 |
| | ACCCCTATGAAA | 1.00E−06 | | 720 |
| | ACACCCACACGA | 1.00E−05 | | 721 |
| | TAGGAGGGTGAG | 1.00E−05 | | 722 |
| | ACCCTACCTCCG | 1.00E−04 | | 723 |
| | CCTGACGTCCTC | 1.00E−04 | | 724 |
| | CGTAGTAGGTGA | 1.00E−04 | | 725 |
| | AGACTGTACATT | 1.00E−02 | | 726 |

“NA” means this motif is without any motif instance in the lncRNA primary sequence.
“Untreated” means this lncRNA did not perform motif scanning.
#, SEQ ID NO:

### REFERENCES

1. Gaj, T., Gersbach, C. A. & Barbas, C. F., 3rd. ZFN, TALEN, and CRISPR/Cas-based methods for genome engineering. Trends Biotechnol. 31, 397-405 (2013).
2. Anzalone, A. V. et al. Search-and-replace genome editing without double-strand breaks or donor DNA. Nature 576, 149-157 (2019).
3. Strecker, J. et al. RNA-guided DNA insertion with CRISPR-associated transposases. Science 365, 48-53 (2019).
4. Rees, H. A. & Liu, D. R. Base editing: precision chemistry on the genome and transcriptome of living cells. Nat. Rev. Genet. 19, 770-788 (2018).
5. Pickar-Oliver, A. & Gersbach, C. A. The next generation of CRISPR-Cas technologies and applications. Nat. Rev. Mol. Cell Biol. 20, 490-507 (2019).
6. Thakore, P. I., Black, J. B., Hilton, I. B. & Gersbach, C. A. Editing the epigenome: technologies for programmable transcription and epigenetic modulation. Nat. Methods 13, 127-137 (2016).
7. Wang, H., La Russa, M. & Qi, L. S. CRISPR/Cas9 in Genome Editing and Beyond. Annu. Rev. Biochem. 85, 227-264 (2016).
8. Jolma, A. et al. Multiplexed massively parallel SELEX for characterization of human transcription factor binding specificities. Genome Res. 20, 861-873 (2010).
9. Valouev, A. et al. Genome-wide analysis of transcription factor binding sites based on ChIP-Seq data. Nat. Methods 5, 829-834 (2008).
10. Bulyk, M. L. Computational prediction of transcription-factor binding site locations.

Genome Biol. 5, 201 (2003).
11. Blanchette, M. et al. Genome-wide computational prediction of transcriptional regulatory modules reveals new insights into human gene expression. Genome Res. 16, 656-668 (2006).
12. Zhao, W. et al. CRISPR-Cas9-mediated functional dissection of 3′-UTRs. Nucleic Acids Res. 45, 10800-10810 (2017).
13. Xue, M., Zhuo, Y. & Shan, B. MicroRNAs, Long Noncoding RNAs, and Their Functions in Human Disease. Methods Mol. Biol. 1617, 1-25 (2017).
14. Li, J., Liang, Q., Song, W. & Marchisio, M. A. Nucleotides upstream of the Kozak sequence strongly influence gene expression in the yeast. J. Biol. Eng. 11, 25 (2017).
15. Pinello, L., Farouni, R. & Yuan, G.-C. Haystack: systematic analysis of the variation of epigenetic states and cell-type specific regulatory elements. Bioinformatics 34, 1930-1933 (2018).
16. Jolma, A. et al. DNA-dependent formation of transcription factor pairs alters their binding specificity. Nature 527, 384-388 (2015).
17. Wang, C., Zhang, M. Q. & Zhang, Z. Computational identification of active enhancers in model organisms. Genomics Proteomics Bioinformatics 11, 142-150 (2013).
18. Yang, J. & Corces, V. G. Chromatin insulators: a role in nuclear organization and gene expression. Adv. Cancer Res. 110, 43-76 (2011).
19. Doni Jayavelu, N., Jajodia, A., Mishra, A. & Hawkins, R. D. Candidate silencer elements for the human and mouse genomes. Nat. Commun. 11, 1061 (2020).
20. Smale, S. T. & Kadonaga, J. T. The RNA polymerase II core promoter. Annu. Rev.

Biochem. 72, 449-479 (2003).
21. Shen, M. W. et al. Predictable and precise template-free CRISPR editing of pathogenic variants. Nature 563, 646-651 (2018).
22. Chen, W. et al. Massively parallel profiling and predictive modeling of the outcomes of CRISPR/Cas9-mediated double-strand break repair. Nucleic Acids Res. 47, 7989-8003 (2019).
23. Nambiar, T. S. et al. Stimulation of CRISPR-mediated homology-directed repair by an engineered RAD18 variant. Nat. Commun. 10, 3395 (2019).
24. Komor, A. C., Kim, Y. B., Packer, M. S., Zuris, J. A. & Liu, D. R. Programmable editing of a target base in genomic DNA without double-stranded DNA cleavage. Nature 533, 420-424 (2016).
25. Gaudelli, N. M. et al. Programmable base editing of AT to GC in genomic DNA without DNA cleavage. Nature 551, 464-471 (2017).
26. Maresca, M., Lin, V. G., Guo, N. & Yang, Y. Obligate ligation-gated recombination (ObLiGaRe): custom-designed nuclease-mediated targeted integration through nonhomologous end joining. Genome Res. 23, 539-546 (2013).
27. Suzuki, K. et al. In vivo genome editing via CRISPR/Cas9 mediated homology-independent targeted integration. Nature 540, 144-149 (2016).
28. Lisse, T. S., Hewison, M. & Adams, J. S. Hormone response element binding proteins: novel regulators of vitamin D and estrogen signaling. Steroids 76, 331-339 (2011).
29. Kribelbauer, J. F., Rastogi, C., Bussemaker, H. J. & Mann, R. S. Low-Affinity Binding Sites and the Transcription Factor Specificity Paradox in Eukaryotes. Annu. Rev. Cell Dev. Biol. 35, 357-379 (2019).
30. Ibarra, I. L. et al. Mechanistic insights into transcription factor cooperativity and its impact on protein-phenotype interactions. Nat. Commun. 11, 124 (2020).
31. Mohaghegh, N. et al. NextPBM: a platform to study cell-specific transcription factor binding and cooperativity. Nucleic Acids Res. 47, e31 (2019).
32. Farley, E. K. et al. Suboptimization of developmental enhancers. Science 350, 325-328 (2015).
33. D'Alessio, A. C. et al. A Systematic Approach to Identify Candidate Transcription Factors that Control Cell Identity. Stem Cell Reports 5, 763-775 (2015).
34. Jones, P. A. & Takai, D. The role of DNA methylation in mammalian epigenetics. Science 293, 1068-1070 (2001).
35. DiNardo, D. N., Butcher, D. T., Robinson, D. P., Archer, T. K. & Rodenhiser, D. I. Functional analysis of CpG methylation in the BRCA1 promoter region. Oncogene 20, 5331-5340 (2001).
36. Guo, Y. et al. Regulation of DNA methylation in human breast cancer. Effect on the urokinase-type plasminogen activator gene production and tumor invasion. J. Biol. Chem. 277, 41571-41579 (2002).
37. Yu, L. et al. Global assessment of promoter methylation in a mouse model of cancer identifies ID4 as a putative tumor-suppressor gene in human leukemia. Nat. Genet. 37, 265-274 (2005).
38. Vierstra, J., Lazar, J., Sandstrom, R. et al. Global reference mapping of human transcription factor footprints. Nature 583, 729-736 (2020).

EXEMPLARY SEQUENCES
Streptococcus pyogenes Cas9, SEQ ID NO: 372
MDKKYSIGLDIGTNSVGWAVITDEYKVPSKKFKVLGNTDRHSIKKNLIGALLFDSGETA
EATRLKRTARRRYTRRKNRICYLQEIFSNEMAKVDDSFFHRLEESFLVEEDKKHERHPI
FGNIVDEVAYHEKYPTIYHLRKKLVDSTDKADLRLIYLALAHMIKFRGHFLIEGDLNPD
NSDVDKLFIQLVQTYNQLFEENPINASGVDAKAILSARLSKSRRLENLIAQLPGEKKNG
LFGNLIALSLGLTPNFKSNFDLAEDAKLQLSKDTYDDDLDNLLAQIGDQYADLFLAAKN
LSDAILLSDILRVNTEITKAPLSASMIKRYDEHHQDLTLLKALVRQQLPEKYKEIFFDQ
SKNGYAGYIDGGASQEEFYKFIKPILEKMDGTEELLVKLNREDLLRKQRTFDNGSIPHQ
IHLGELHAILRRQEDFYPFLKDNREKIEKILTFRIPYYVGPLARGNSRFAWMTRKSEET
ITPWNFEEVVDKGASAQSFIERMTNFDKNLPNEKVLPKHSLLYEYFTVYNELTKVKYVT
EGMRKPAFLSGEQKKAIVDLLFKTNRKVTVKQLKEDYFKKIECFDSVEISGVEDRFNAS
LGTYHDLLKIIKDKDFLDNEENEDILEDIVLTLTLFEDREMIEERLKTYAHLFDDKVMK
QLKRRRYTGWGRLSRKLINGIRDKQSGKTILDFLKSDGFANRNFMQLIHDDSLTFKEDI
QKAQVSGQGDSLHEHIANLAGSPAIKKGILQTVKVVDELVKVMGRHKPENIVIEMAREN
QTTQKGQKNSRERMKRIEEGIKELGSQILKEHPVENTQLQNEKLYLYYLQNGRDMYVDQ
ELDINRLSDYDVDHIVPQSFLKDDSIDNKVLTRSDKNRGKSDNVPSEEVVKKMKNYWRQ
LLNAKLITQRKFDNLTKAERGGLSELDKAGFIKRQLVETRQITKHVAQILDSRMNTKYD
ENDKLIREVKVITLKSKLVSDFRKDFQFYKVREINNYHHAHDAYLNAVVGTALIKKYPK
LESEFVYGDYKVYDVRKMIAKSEQEIGKATAKYFFYSNIMNFFKTEITLANGEIRKRPL
IETNGETGEIVWDKGRDFATVRKVLSMPQVNIVKKTEVQTGGFSKESILPKRNSDKLIA
RKKDWDPKKYGGFDSPTVAYSVLVVAKVEKGKSKKLKSVKELLGITIMERSSFEKNPID
FLEAKGYKEVKKDLIIKLPKYSLFELENGRKRMLASAGELQKGNELALPSKYVNFLYLA
SHYEKLKGSPEDNEQKQLFVEQHKHYLDEIIEQISEFSKRVILADANLDKVLSAYNKHR
DKPIREQAENIIHLFTLTNLGAPAAFKYFDTTIDRKRYTSTKEVLDATLIHQSITGLYE
TRIDLSQLGGDGSPKKKRKVSSDYKDHDGDYKDHDIDYKDDDDK
SpRY Cas9 variant, SEQ ID NO: 373
MDKKYSIGLAIGTNSVGWAVITDEYKVPSKKFKVLGNTDRHSIKKNLIGALLFDSGETA
ERTRLKRTARRRYTRRKNRICYLQEIFSNEMAKVDDSFFHRLEESFLVEEDKKHERHPI
FGNIVDEVAYHEKYPTIYHLRKKLVDSTDKADLRLIYLALAHMIKFRGHFLIEGDLNPD
NSDVDKLFIQLVQTYNQLFEENPINASGVDAKAILSARLSKSRRLENLIAQLPGEKKNG
LFGNLIALSLGLTPNFKSNFDLAEDAKLQLSKDTYDDDLDNLLAQIGDQYADLFLAAKN
LSDAILLSDILRVNTEITKAPLSASMIKRYDEHHQDLTLLKALVRQQLPEKYKEIFFDQ
SKNGYAGYIDGGASQEEFYKFIKPILEKMDGTEELLVKLNREDLLRKQRTFDNGSIPHQ
IHLGELHAILRRQEDFYPFLKDNREKIEKILTFRIPYYVGPLARGNSRFAWMTRKSEET
ITPWNFEEVVDKGASAQSFIERMTNFDKNLPNEKVLPKHSLLYEYFTVYNELTKVKYVT
EGMRKPAFLSGEQKKAIVDLLFKTNRKVTVKQLKEDYFKKIECFDSVEISGVEDRFNAS
LGTYHDLLKIIKDKDFLDNEENEDILEDIVLTLTLFEDREMIEERLKTYAHLFDDKVMK
QLKRRRYTGWGRLSRKLINGIRDKQSGKTILDFLKSDGFANRNFMQLIHDDSLTFKEDI
QKAQVSGQGDSLHEHIANLAGSPAIKKGILQTVKVVDELVKVMGRHKPENIVIEMAREN
QTTQKGQKNSRERMKRIEEGIKELGSQILKEHPVENTQLQNEKLYLYYLQNGRDMYVDQ
ELDINRLSDYDVDHIVPQSFLKDDSIDNKVLTRSDKNRGKSDNVPSEEVVKKMKNYWRQ
LLNAKLITQRKFDNLTKAERGGLSELDKAGFIKRQLVETRQITKHVAQILDSRMNTKYD
ENDKLIREVKVITLKSKLVSDFRKDFQFYKVREINNYHHAHDAYLNAVVGTALIKKYPK
LESEFVYGDYKVYDVRKMIAKSEQEIGKATAKYFFYSNIMNFFKTEITLANGEIRKRPL
IETNGETGEIVWDKGRDFATVRKVLSMPQVNIVKKTEVQTGGFSKESIRPKRNSDKLIA
RKKDWDPKKYGGFLWPTVAYSVLVVAKVEKGKSKKLKSVKELLGITIMERSSFEKNPID
FLEAKGYKEVKKDLIIKLPKYSLFELENGRKRMLASAKQLQKGNELALPSKYVNFLYLA
SHYEKLKGSPEDNEQKQLFVEQHKHYLDEIIEQISEFSKRVILADANLDKVLSAYNKHR
DKPIREQAENIIHLFTLTRLGAPRAFKYFDTTIDPKQYRSTKEVLDATLIHQSITGLYE
TRIDLSQLGGD
SpG Cas9 variant, SEQ ID NO: 374
MDKKYSIGLAIGTNSVGWAVITDEYKVPSKKFKVLGNTDRHSIKKNLIGALLFDSGETA
EATRLKRTARRRYTRRKNRICYLQEIFSNEMAKVDDSFFHRLEESFLVEEDKKHERHPI
FGNIVDEVAYHEKYPTIYHLRKKLVDSTDKADLRLIYLALAHMIKFRGHFLIEGDLNPD
NSDVDKLFIQLVQTYNQLFEENPINASGVDAKAILSARLSKSRRLENLIAQLPGEKKNG
LFGNLIALSLGLTPNFKSNFDLAEDAKLQLSKDTYDDDLDNLLAQIGDQYADLFLAAKN
LSDAILLSDILRVNTEITKAPLSASMIKRYDEHHQDLTLLKALVRQQLPEKYKEIFFDQ
SKNGYAGYIDGGASQEEFYKFIKPILEKMDGTEELLVKLNREDLLRKQRTFDNGSIPHQ
IHLGELHAILRRQEDFYPFLKDNREKIEKILTFRIPYYVGPLARGNSRFAWMTRKSEET
ITPWNFEEVVDKGASAQSFIERMTNFDKNLPNEKVLPKHSLLYEYFTVYNELTKVKYVT
EGMRKPAFLSGEQKKAIVDLLFKTNRKVTVKQLKEDYFKKIECFDSVEISGVEDRFNAS
LGTYHDLLKIIKDKDFLDNEENEDILEDIVLTLTLFEDREMIEERLKTYAHLFDDKVMK
QLKRRRYTGWGRLSRKLINGIRDKQSGKTILDFLKSDGFANRNFMQLIHDDSLTFKEDI
QKAQVSGQGDSLHEHIANLAGSPAIKKGILQTVKVVDELVKVMGRHKPENIVIEMAREN
QTTQKGQKNSRERMKRIEEGIKELGSQILKEHPVENTQLQNEKLYLYYLQNGRDMYVDQ
ELDINRLSDYDVDHIVPQSFLKDDSIDNKVLTRSDKNRGKSDNVPSEEVVKKMKNYWRQ
LLNAKLITQRKFDNLTKAERGGLSELDKAGFIKRQLVETRQITKHVAQILDSRMNTKYD
ENDKLIREVKVITLKSKLVSDFRKDFQFYKVREINNYHHAHDAYLNAVVGTALIKKYPK
LESEFVYGDYKVYDVRKMIAKSEQEIGKATAKYFFYSNIMNFFKTEITLANGEIRKRPL
IETNGETGEIVWDKGRDFATVRKVLSMPQVNIVKKTEVQTGGFSKESILPKRNSDKLIA
RKKDWDPKKYGGFLWPTVAYSVLVVAKVEKGKSKKLKSVKELLGITIMERSSFEKNPID
FLEAKGYKEVKKDLIIKLPKYSLFELENGRKRMLASAKQLQKGNELALPSKYVNFLYLA
SHYEKLKGSPEDNEQKQLFVEQHKHYLDEIIEQISEFSKRVILADANLDKVLSAYNKHR
DKPIREQAENIIHLFTLTNLGAPAAFKYFDTTIDRKQYRSTKEVLDATLIHQSITGLYE
TRIDLSQLGGD
BE1 for Mammalian expression (rAPOBEC1-XTEN-
dCas9-NLS), SEQ ID NO: 375
MSSETGPVAVDPTLRRRIEPHEFEVFFDPRELRKETCLLYEINWGGRHSIWRHTSQNTN
KHVEVNFIEKFTTERYFCPNTRCSITWFLSWSPCGECSRAITEFLSRYPHVTLFIYIAR
LYHHADPRNRQGLRDLISSGVTIQIMTEQESGYCWRNFVNYSPSNEAHWPRYPHLWVRL
YVLELYCIILGLPPCLNILRRKQPQLTFFTIALQSCHYQRLPPHILWATGLKSGSETPG
TSESATPESDKKYSIGLAIGTNSVGWAVITDEYKVPSKKFKVLGNTDRHSIKKNLIGAL
LFDSGETAEATRLKRTARRRYTRRKNRICYLQEIFSNEMAKVDDSFFHRLEESFLVEED
KKHERHPIFGNIVDEVAYHEKYPTIYHLRKKLVDSTDKADLRLIYLALAHMIKFRGHFL
IEGDLNPDNSDVDKLFIQLVQTYNQLFEENPINASGVDAKAILSARLSKSRRLENLIAQ
LPGEKKNGLFGNLIALSLGLTPNFKSNFDLAEDAKLQLSKDTYDDDLDNLLAQIGDQYA
DLFLAAKNLSDAILLSDILRVNTEITKAPLSASMIKRYDEHHQDLTLLKALVRQQLPEK
YKEIFFDQSKNGYAGYIDGGASQEEFYKFIKPILEKMDGTEELLVKLNREDLLRKQRTF
DNGSIPHQIHLGELHAILRRQEDFYPFLKDNREKIEKILTFRIPYYVGPLARGNSRFAW
MTRKSEETITPWNFEEVVDKGASAQSFIERMTNFDKNLPNEKVLPKHSLLYEYFTVYNE
LTKVKYVTEGMRKPAFLSGEQKKAIVDLLFKTNRKVTVKQLKEDYFKKIECFDSVEISG
VEDRFNASLGTYHDLLKIIKDKDFLDNEENEDILEDIVLTLTLFEDREMIEERLKTYAH
LFDDKVMKQLKRRRYTGWGRLSRKLINGIRDKQSGKTILDFLKSDGFANRNFMQLIHDD
SLTFKEDIQKAQVSGQGDSLHEHIANLAGSPAIKKGILQTVKVVDELVKVMGRHKPENI
VIEMARENQTTQKGQKNSRERMKRIEEGIKELGSQILKEHPVENTQLQNEKLYLYYLQN
GRDMYVDQELDINRLSDYDVDAIVPQSFLKDDSIDNKVLTRSDKNRGKSDNVPSEEVVK
KMKNYWRQLLNAKLITQRKFDNLTKAERGGLSELDKAGFIKRQLVETRQITKHVAQILD
SRMNTKYDENDKLIREVKVITLKSKLVSDFRKDFQFYKVREINNYHHAHDAYLNAVVGT
ALIKKYPKLESEFVYGDYKVYDVRKMIAKSEQEIGKATAKYFFYSNIMNFFKTEITLAN
GEIRKRPLIETNGETGEIVWDKGRDFATVRKVLSMPQVNIVKKTEVQTGGFSKESILPK
RNSDKLIARKKDWDPKKYGGFDSPTVAYSVLVVAKVEKGKSKKLKSVKELLGITIMERS
SFEKNPIDFLEAKGYKEVKKDLIIKLPKYSLFELENGRKRMLASAGELQKGNELALPSK
YVNFLYLASHYEKLKGSPEDNEQKQLFVEQHKHYLDEIIEQISEFSKRVILADANLDKV
LSAYNKHRDKPIREQAENIIHLFTLTNLGAPAAFKYFDTTIDRKRYTSTKEVLDATLIH
QSITGLYETRIDLSQLGGDSGGSPKKKRKV
BE2 (rAPOBEC1-XTEN-dCas9-UGI-NLS) SEQ ID NO: 376
MSSETGPVAVDPTLRRRIEPHEFEVFFDPRELRKETCLLYEINWGGRHSIWRHTSQNTN
KHVEVNFIEKFTTERYFCPNTRCSITWFLSWSPCGECSRAITEFLSRYPHVTLFIYIAR
LYHHADPRNRQGLRDLISSGVTIQIMTEQESGYCWRNFVNYSPSNEAHWPRYPHLWVRL
YVLELYCIILGLPPCLNILRRKQPQLTFFTIALQSCHYQRLPPHILWATGLKSGSETPG
TSESATPESDKKYSIGLAIGTNSVGWAVITDEYKVPSKKFKVLGNTDRHSIKKNLIGAL
LFDSGETAEATRLKRTARRRYTRRKNRICYLQEIFSNEMAKVDDSFFHRLEESFLVEED
KKHERHPIFGNIVDEVAYHEKYPTIYHLRKKLVDSTDKADLRLIYLALAHMIKFRGHFL
IEGDLNPDNSDVDKLFIQLVQTYNQLFEENPINASGVDAKAILSARLSKSRRLENLIAQ
LPGEKKNGLFGNLIALSLGLTPNFKSNFDLAEDAKLQLSKDTYDDDLDNLLAQIGDQYA
DLFLAAKNLSDAILLSDILRVNTEITKAPLSASMIKRYDEHHQDLTLLKALVRQQLPEK
YKEIFFDQSKNGYAGYIDGGASQEEFYKFIKPILEKMDGTEELLVKLNREDLLRKQRTF
DNGSIPHQIHLGELHAILRRQEDFYPFLKDNREKIEKILTFRIPYYVGPLARGNSRFAW
MTRKSEETITPWNFEEVVDKGASAQSFIERMTNFDKNLPNEKVLPKHSLLYEYFTVYNE
LTKVKYVTEGMRKPAFLSGEQKKAIVDLLFKTNRKVTVKQLKEDYFKKIECFDSVEISG
VEDRFNASLGTYHDLLKIIKDKDFLDNEENEDILEDIVLTLTLFEDREMIEERLKTYAH
LFDDKVMKQLKRRRYTGWGRLSRKLINGIRDKQSGKTILDFLKSDGFANRNFMQLIHDD
SLTFKEDIQKAQVSGQGDSLHEHIANLAGSPAIKKGILQTVKVVDELVKVMGRHKPENI
VIEMARENQTTQKGQKNSRERMKRIEEGIKELGSQILKEHPVENTQLQNEKLYLYYLQN
GRDMYVDQELDINRLSDYDVDAIVPQSFLKDDSIDNKVLTRSDKNRGKSDNVPSEEVVK
KMKNYWRQLLNAKLITQRKFDNLTKAERGGLSELDKAGFIKRQLVETRQITKHVAQILD
SRMNTKYDENDKLIREVKVITLKSKLVSDFRKDFQFYKVREINNYHHAHDAYLNAVVGT
ALIKKYPKLESEFVYGDYKVYDVRKMIAKSEQEIGKATAKYFFYSNIMNFFKTEITLAN
GEIRKRPLIETNGETGEIVWDKGRDFATVRKVLSMPQVNIVKKTEVQTGGFSKESILPK
RNSDKLIARKKDWDPKKYGGFDSPTVAYSVLVVAKVEKGKSKKLKSVKELLGITIMERS
SFEKNPIDFLEAKGYKEVKKDLIIKLPKYSLFELENGRKRMLASAGELQKGNELALPSK
YVNFLYLASHYEKLKGSPEDNEQKQLFVEQHKHYLDEIIEQISEFSKRVILADANLDKV
LSAYNKHRDKPIREQAENIIHLFTLTNLGAPAAFKYFDTTIDRKRYTSTKEVLDATLIH
QSITGLYETRIDLSQLGGDSGGSTNLSDIIEKETGKQLVIQESILMLPEEVEEVIGNKP
ESDILVHTAYDESTDENVMLLTSDAPEYKPWALVIQDSNGENKIKMLSGGSPKKKRKV
BE3 (rAPOBEC1-XTEN-Cas9n-UGI-NLS) SEQ ID NO: 377
MSSETGPVAVDPTLRRRIEPHEFEVFFDPRELRKETCLLYEINWGGRHSIWRHTSQNTN
KHVEVNFIEKFTTERYFCPNTRCSITWFLSWSPCGECSRAITEFLSRYPHVTLFIYIAR
LYHHADPRNRQGLRDLISSGVTIQIMTEQESGYCWRNFVNYSPSNEAHWPRYPHLWVRL
YVLELYCIILGLPPCLNILRRKQPQLTFFTIALQSCHYQRLPPHILWATGLKSGSETPG
TSESATPESDKKYSIGLAIGTNSVGWAVITDEYKVPSKKFKVLGNTDRHSIKKNLIGAL
LFDSGETAEATRLKRTARRRYTRRKNRICYLQEIFSNEMAKVDDSFFHRLEESFLVEED
KKHERHPIFGNIVDEVAYHEKYPTIYHLRKKLVDSTDKADLRLIYLALAHMIKFRGHFL
IEGDLNPDNSDVDKLFIQLVQTYNQLFEENPINASGVDAKAILSARLSKSRRLENLIAQ
LPGEKKNGLFGNLIALSLGLTPNFKSNFDLAEDAKLQLSKDTYDDDLDNLLAQIGDQYA
DLFLAAKNLSDAILLSDILRVNTEITKAPLSASMIKRYDEHHQDLTLLKALVRQQLPEK
YKEIFFDQSKNGYAGYIDGGASQEEFYKFIKPILEKMDGTEELLVKLNREDLLRKQRTF
DNGSIPHQIHLGELHAILRRQEDFYPFLKDNREKIEKILTFRIPYYVGPLARGNSRFAW
MTRKSEETITPWNFEEVVDKGASAQSFIERMTNFDKNLPNEKVLPKHSLLYEYFTVYNE
LTKVKYVTEGMRKPAFLSGEQKKAIVDLLFKTNRKVTVKQLKEDYFKKIECFDSVEISG
VEDRFNASLGTYHDLLKIIKDKDFLDNEENEDILEDIVLTLTLFEDREMIEERLKTYAH
LFDDKVMKQLKRRRYTGWGRLSRKLINGIRDKQSGKTILDFLKSDGFANRNFMQLIHDD
SLTFKEDIQKAQVSGQGDSLHEHIANLAGSPAIKKGILQTVKVVDELVKVMGRHKPENI
VIEMARENQTTQKGQKNSRERMKRIEEGIKELGSQILKEHPVENTQLQNEKLYLYYLQN
GRDMYVDQELDINRLSDYDVDHIVPQSFLKDDSIDNKVLTRSDKNRGKSDNVPSEEVVK
KMKNYWRQLLNAKLITQRKFDNLTKAERGGLSELDKAGFIKRQLVETRQITKHVAQILD
SRMNTKYDENDKLIREVKVITLKSKLVSDFRKDFQFYKVREINNYHHAHDAYLNAVVGT
ALIKKYPKLESEFVYGDYKVYDVRKMIAKSEQEIGKATAKYFFYSNIMNFFKTEITLAN
GEIRKRPLIETNGETGEIVWDKGRDFATVRKVLSMPQVNIVKKTEVQTGGFSKESILPK
RNSDKLIARKKDWDPKKYGGFDSPTVAYSVLVVAKVEKGKSKKLKSVKELLGITIMERS
SFEKNPIDFLEAKGYKEVKKDLIIKLPKYSLFELENGRKRMLASAGELQKGNELALPSK
YVNFLYLASHYEKLKGSPEDNEQKQLFVEQHKHYLDEIIEQISEFSKRVILADANLDKV
LSAYNKHRDKPIREQAENIIHLFTLTNLGAPAAFKYFDTTIDRKRYTSTKEVLDATLIH
QSITGLYETRIDLSQLGGDSGGSTNLSDIIEKETGKQLVIQESILMLPEEVEEVIGNKP
ESDILVHTAYDESTDENVMLLTSDAPEYKPWALVIQDSNGENKIKMLSGGSPKKKRKV
CDA1-BE3: SEQ ID NO: 378
MTDAEYVRIHEKLDIYTFKKQFFNNKKSVSHRCYVLFELKRRGERRACFWGYAVNKPQS
GTERGIHAEIFSIRKVEEYLRDNPGQFTINWYSSWSPCADCAEKILEWYNQELRGNGHT
LKIWACKLYYEKNARNQIGLWNLRDNGVGLNVMVSEHYQCCRKIFIQSSHNQLNENRWL
EKTLKRAEKRRSELSIMIQVKILHTTKSPAVSGSETPGTSESATPESDKKYSIGLAIGT
NSVGWAVITDEYKVPSKKFKVLGNTDRHSIKKNLIGALLFDSGETAEATRLKRTARRRY
TRRKNRICYLQEIFSNEMAKVDDSFFHRLEESFLVEEDKKHERHPIFGNIVDEVAYHEK
YPTIYHLRKKLVDSTDKADLRLIYLALAHMIKFRGHFLIEGDLNPDNSDVDKLFIQLVQ
TYNQLFEENPINASGVDAKAILSARLSKSRRLENLIAQLPGEKKNGLFGNLIALSLGLT
PNFKSNFDLAEDAKLQLSKDTYDDDLDNLLAQIGDQYADLFLAAKNLSDAILLSDILRV
NTEITKAPLSASMIKRYDEHHQDLTLLKALVRQQLPEKYKEIFFDQSKNGYAGYIDGGA
SQEEFYKFIKPILEKMDGTEELLVKLNREDLLRKQRTFDNGSIPHQIHLGELHAILRRQ
EDFYPFLKDNREKIEKILTFRIPYYVGPLARGNSRFAWMTRKSEETITPWNFEEVVDKG
ASAQSFIERMTNFDKNLPNEKVLPKHSLLYEYFTVYNELTKVKYVTEGMRKPAFLSGEQ
KKAIVDLLFKTNRKVTVKQLKEDYFKKIECFDSVEISGVEDRFNASLGTYHDLLKIIKD
KDFLDNEENEDILEDIVLTLTLFEDREMIEERLKTYAHLFDDKVMKQLKRRRYTGWGRL
SRKLINGIRDKQSGKTILDFLKSDGFANRNFMQLIHDDSLTFKEDIQKAQVSGQGDSLH
EHIANLAGSPAIKKGILQTVKVVDELVKVMGRHKPENIVIEMARENQTTQKGQKNSRER
MKRIEEGIKELGSQILKEHPVENTQLQNEKLYLYYLQNGRDMYVDQELDINRLSDYDVD
HIVPQSFLKDDSIDNKVLTRSDKNRGKSDNVPSEEVVKKMKNYWRQLLNAKLITQRKFD
NLTKAERGGLSELDKAGFIKRQLVETRQITKHVAQILDSRMNTKYDENDKLIREVKVIT
LKSKLVSDFRKDFQFYKVREINNYHHAHDAYLNAVVGTALIKKYPKLESEFVYGDYKVY
DVRKMIAKSEQEIGKATAKYFFYSNIMNFFKTEITLANGEIRKRPLIETNGETGEIVWD
KGRDFATVRKVLSMPQVNIVKKTEVQTGGFSKESILPKRNSDKLIARKKDWDPKKYGGF
DSPTVAYSVLVVAKVEKGKSKKLKSVKELLGITIMERSSFEKNPIDFLEAKGYKEVKKD
LIIKLPKYSLFELENGRKRMLASAGELQKGNELALPSKYVNFLYLASHYEKLKGSPEDN
EQKQLFVEQHKHYLDEIIEQISEFSKRVILADANLDKVLSAYNKHRDKPIREQAENIIH
LFTLTNLGAPAAFKYFDTTIDRKRYTSTKEVLDATLIHQSITGLYETRIDLSQLGGDSG
GSTNLSDIIEKETGKQLVIQESILMLPEEVEEVIGNKPESDILVHTAYDESTDENVMLL
TSDAPEYKPWALVIQDSNGENKIKMLSGGSPKKKRKV
AID-BE3: SEQ ID NO: 379
MDSLLMNRRKFLYQFKNVRWAKGRRETYLCYVVKRRDSATSFSLDFGYLRNKNGCHVEL
LFLRYISDWDLDPGRCYRVTWFTSWSPCYDCARHVADFLRGNPNLSLRIFTARLYFCED
RKAEPEGLRRLHRAGVQIAIMTFKDYFYCWNTFVENHERTFKAWEGLHENSVRLSRQLR
RILLPLYEVDDLRDAFRTLGLSGSETPGTSESATPESDKKYSIGLAIGTNSVGWAVITD
EYKVPSKKFKVLGNTDRHSIKKNLIGALLFDSGETAEATRLKRTARRRYTRRKNRICYL
QEIFSNEMAKVDDSFFHRLEESFLVEEDKKHERHPIFGNIVDEVAYHEKYPTIYHLRKK
LVDSTDKADLRLIYLALAHMIKFRGHFLIEGDLNPDNSDVDKLFIQLVQTYNQLFEENP
INASGVDAKAILSARLSKSRRLENLIAQLPGEKKNGLFGNLIALSLGLTPNFKSNFDLA
EDAKLQLSKDTYDDDLDNLLAQIGDQYADLFLAAKNLSDAILLSDILRVNTEITKAPLS
ASMIKRYDEHHQDLTLLKALVRQQLPEKYKEIFFDQSKNGYAGYIDGGASQEEFYKFIK
PILEKMDGTEELLVKLNREDLLRKQRTFDNGSIPHQIHLGELHAILRRQEDFYPFLKDN
REKIEKILTFRIPYYVGPLARGNSRFAWMTRKSEETITPWNFEEVVDKGASAQSFIERM
TNFDKNLPNEKVLPKHSLLYEYFTVYNELTKVKYVTEGMRKPAFLSGEQKKAIVDLLFK
TNRKVTVKQLKEDYFKKIECFDSVEISGVEDRFNASLGTYHDLLKIIKDKDFLDNEENE
DILEDIVLTLTLFEDREMIEERLKTYAHLFDDKVMKQLKRRRYTGWGRLSRKLINGIRD
KQSGKTILDFLKSDGFANRNFMQLIHDDSLTFKEDIQKAQVSGQGDSLHEHIANLAGSP
AIKKGILQTVKVVDELVKVMGRHKPENIVIEMARENQTTQKGQKNSRERMKRIEEGIKE
LGSQILKEHPVENTQLQNEKLYLYYLQNGRDMYVDQELDINRLSDYDVDHIVPQSFLKD
DSIDNKVLTRSDKNRGKSDNVPSEEVVKKMKNYWRQLLNAKLITQRKFDNLTKAERGGL
SELDKAGFIKRQLVETRQITKHVAQILDSRMNTKYDENDKLIREVKVITLKSKLVSDFR
KDFQFYKVREINNYHHAHDAYLNAVVGTALIKKYPKLESEFVYGDYKVYDVRKMIAKSE
QEIGKATAKYFFYSNIMNFFKTEITLANGEIRKRPLIETNGETGEIVWDKGRDFATVRK
VLSMPQVNIVKKTEVQTGGFSKESILPKRNSDKLIARKKDWDPKKYGGFDSPTVAYSVL
VVAKVEKGKSKKLKSVKELLGITIMERSSFEKNPIDFLEAKGYKEVKKDLIIKLPKYSL
FELENGRKRMLASAGELQKGNELALPSKYVNFLYLASHYEKLKGSPEDNEQKQLFVEQH
KHYLDEIIEQISEFSKRVILADANLDKVLSAYNKHRDKPIREQAENIIHLFTLTNLGAP
AAFKYFDTTIDRKRYTSTKEVLDATLIHQSITGLYETRIDLSQLGGDSGGSTNLSDIIE
KETGKQLVIQESILMLPEEVEEVIGNKPESDILVHTAYDESTDENVMLLTSDAPEYKPW
ALVIQDSNGENKIKMLSGGSPKKKRKV
BE3-Gam: SEQ ID NO: 380
MAKPAKRIKSAAAAYVPQNRDAVITDIKRIGDLQREASRLETEMNDAIAEITEKFAARI
APIKTDIETLSKGVQGWCEANRDELTNGGKVKTANLVTGDVSWRVRPPSVSIRGMDAVM
ETLERLGLQRFIRTKQEINKEAILLEPKAVAGVAGITVKSGIEDFSIIPFEQEAGISGS
ETPGTSESATPESSSETGPVAVDPTLRRRIEPHEFEVFFDPRELRKETCLLYEINWGGR
HSIWRHTSQNTNKHVEVNFIEKFTTERYFCPNTRCSITWFLSWSPCGECSRAITEFLSR
YPHVTLFIYIARLYHHADPRNRQGLRDLISSGVTIQIMTEQESGYCWRNFVNYSPSNEA
HWPRYPHLWVRLYVLELYCIILGLPPCLNILRRKQPQLTFFTIALQSCHYQRLPPHILW
ATGLKSGSETPGTSESATPESDKKYSIGLAIGTNSVGWAVITDEYKVPSKKFKVLGNTD
RHSIKKNLIGALLFDSGETAEATRLKRTARRRYTRRKNRICYLQEIFSNEMAKVDDSFF
HRLEESFLVEEDKKHERHPIFGNIVDEVAYHEKYPTIYHLRKKLVDSTDKADLRLIYLA
LAHMIKFRGHFLIEGDLNPDNSDVDKLFIQLVQTYNQLFEENPINASGVDAKAILSARL
SKSRRLENLIAQLPGEKKNGLFGNLIALSLGLTPNFKSNFDLAEDAKLQLSKDTYDDDL
DNLLAQIGDQYADLFLAAKNLSDAILLSDILRVNTEITKAPLSASMIKRYDEHHQDLTL
LKALVRQQLPEKYKEIFFDQSKNGYAGYIDGGASQEEFYKFIKPILEKMDGTEELLVKL
NREDLLRKQRTFDNGSIPHQIHLGELHAILRRQEDFYPFLKDNREKIEKILTFRIPYYV
GPLARGNSRFAWMTRKSEETITPWNFEEVVDKGASAQSFIERMTNFDKNLPNEKVLPKH
SLLYEYFTVYNELTKVKYVTEGMRKPAFLSGEQKKAIVDLLFKTNRKVTVKQLKEDYFK
KIECFDSVEISGVEDRFNASLGTYHDLLKIIKDKDFLDNEENEDILEDIVLTLTLFEDR
EMIEERLKTYAHLFDDKVMKQLKRRRYTGWGRLSRKLINGIRDKQSGKTILDFLKSDGF
ANRNFMQLIHDDSLTFKEDIQKAQVSGQGDSLHEHIANLAGSPAIKKGILQTVKVVDEL
VKVMGRHKPENIVIEMARENQTTQKGQKNSRERMKRIEEGIKELGSQILKEHPVENTQL
QNEKLYLYYLQNGRDMYVDQELDINRLSDYDVDHIVPQSFLKDDSIDNKVLTRSDKNRG
KSDNVPSEEVVKKMKNYWRQLLNAKLITQRKFDNLTKAERGGLSELDKAGFIKRQLVET
RQITKHVAQILDSRMNTKYDENDKLIREVKVITLKSKLVSDFRKDFQFYKVREINNYHH
AHDAYLNAVVGTALIKKYPKLESEFVYGDYKVYDVRKMIAKSEQEIGKATAKYFFYSNI
MNFFKTEITLANGEIRKRPLIETNGETGEIVWDKGRDFATVRKVLSMPQVNIVKKTEVQ
TGGFSKESILPKRNSDKLIARKKDWDPKKYGGFDSPTVAYSVLVVAKVEKGKSKKLKSV
KELLGITIMERSSFEKNPIDFLEAKGYKEVKKDLIIKLPKYSLFELENGRKRMLASAGE
LQKGNELALPSKYVNFLYLASHYEKLKGSPEDNEQKQLFVEQHKHYLDEIIEQISEFSK
RVILADANLDKVLSAYNKHRDKPIREQAENIIHLFTLTNLGAPAAFKYFDTTIDRKRYT
STKEVLDATLIHQSITGLYETRIDLSQLGGDSGGSTNLSDIIEKETGKQLVIQESILML
PEEVEEVIGNKPESDILVHTAYDESTDENVMLLTSDAPEYKPWALVIQDSNGENKIKML
SGGSPKKKRKV
SaBE3-Gam: SEQ ID NO: 381
MAKPAKRIKSAAAAYVPQNRDAVITDIKRIGDLQREASRLETEMNDAIAEITEKFAARI
APIKTDIETLSKGVQGWCEANRDELTNGGKVKTANLVTGDVSWRVRPPSVSIRGMDAVM
ETLERLGLQRFIRTKQEINKEAILLEPKAVAGVAGITVKSGIEDFSIIPFEQEAGISGS
ETPGTSESATPESSSETGPVAVDPTLRRRIEPHEFEVFFDPRELRKETCLLYEINWGGR
HSIWRHTSQNTNKHVEVNFIEKFTTERYFCPNTRCSITWFLSWSPCGECSRAITEFLSR
YPHVTLFIYIARLYHHADPRNRQGLRDLISSGVTIQIMTEQESGYCWRNFVNYSPSNEA
HWPRYPHLWVRLYVLELYCIILGLPPCLNILRRKQPQLTFFTIALQSCHYQRLPPHILW
ATGLKSGSETPGTSESATPESGKRNYILGLAIGITSVGYGIIDYETRDVIDAGVRLFKE
ANVENNEGRRSKRGARRLKRRRRHRIQRVKKLLFDYNLLTDHSELSGINPYEARVKGLS
QKLSEEEFSAALLHLAKRRGVHNVNEVEEDTGNELSTKEQISRNSKALEEKYVAELQLE
RLKKDGEVRGSINRFKTSDYVKEAKQLLKVQKAYHQLDQSFIDTYIDLLETRRTYYEGP
GEGSPFGWKDIKEWYEMLMGHCTYFPEELRSVKYAYNADLYNALNDLNNLVITRDENEK
LEYYEKFQIIENVFKQKKKPTLKQIAKEILVNEEDIKGYRVTSTGKPEFTNLKVYHDIK
DITARKEIIENAELLDQIAKILTIYQSSEDIQEELTNLNSELTQEEIEQISNLKGYTGT
HNLSLKAINLILDELWHTNDNQIAIFNRLKLVPKKVDLSQQKEIPTTLVDDFILSPVVK
RSFIQSIKVINAIIKKYGLPNDIIIELAREKNSKDAQKMINEMQKRNRQTNERIEEIIR
TTGKENAKYLIEKIKLHDMQEGKCLYSLEAIPLEDLLNNPFNYEVDHIIPRSVSFDNSF
NNKVLVKQEENSKKGNRTPFQYLSSSDSKISYETFKKHILNLAKGKGRISKTKKEYLLE
ERDINRFSVQKDFINRNLVDTRYATRGLMNLLRSYFRVNNLDVKVKSINGGFTSFLRRK
WKFKKERNKGYKHHAEDALIIANADFIFKEWKKLDKAKKVMENQMFEEKQAESMPEIET
EQEYKEIFITPHQIKHIKDFKDYKYSHRVDKKPNRELINDTLYSTRKDDKGNTLIVNNL
NGLYDKDNDKLKKLINKSPEKLLMYHHDPQTYQKLKLIMEQYGDEKNPLYKYYEETGNY
LTKYSKKDNGPVIKKIKYYGNKLNAHLDITDDYPNSRNKVVKLSLKPYRFDVYLDNGVY
KFVTVKNLDVIKKENYYEVNSKCYEEAKKLKKISNQAEFIASFYNNDLIKINGELYRVI
GVNNDLLNRIEVNMIDITYREYLENMNDKRPPRIIKTIASKTQSIKKYSTDILGNLYEV
KSKKHPQIIKKGGSPKKKRKVSSDYKDHDGDYKDHDIDYKDDDDKSGGSTNLSDIIEKE
TGKQLVIQESILMLPEEVEEVIGNKPESDILVHTAYDESTDENVMLLTSDAPEYKPWAL
VIQDSNGENKIKMLSGGSPKKKRKV
BE4: SEQ ID NO: 382
MSSETGPVAVDPTLRRRIEPHEFEVFFDPRELRKETCLLYEINWGGRHSIWRHTSQNTN
KHVEVNFIEKFTTERYFCPNTRCSITWFLSWSPCGECSRAITEFLSRYPHVTLFIYIAR
LYHHADPRNRQGLRDLISSGVTIQIMTEQESGYCWRNFVNYSPSNEAHWPRYPHLWVRL
YVLELYCIILGLPPCLNILRRKQPQLTFFTIALQSCHYQRLPPHILWATGLKSGGSSGG
SSGSETPGTSESATPESSGGSSGGSDKKYSIGLAIGTNSVGWAVITDEYKVPSKKFKVL
GNTDRHSIKKNLIGALLFDSGETAEATRLKRTARRRYTRRKNRICYLQEIFSNEMAKVD
DSFFHRLEESFLVEEDKKHERHPIFGNIVDEVAYHEKYPTIYHLRKKLVDSTDKADLRL
IYLALAHMIKFRGHFLIEGDLNPDNSDVDKLFIQLVQTYNQLFEENPINASGVDAKAIL
SARLSKSRRLENLIAQLPGEKKNGLFGNLIALSLGLTPNFKSNFDLAEDAKLQLSKDTY
DDDLDNLLAQIGDQYADLFLAAKNLSDAILLSDILRVNTEITKAPLSASMIKRYDEHHQ
DLTLLKALVRQQLPEKYKEIFFDQSKNGYAGYIDGGASQEEFYKFIKPILEKMDGTEEL
LVKLNREDLLRKQRTFDNGSIPHQIHLGELHAILRRQEDFYPFLKDNREKIEKILTFRI
PYYVGPLARGNSRFAWMTRKSEETITPWNFEEVVDKGASAQSFIERMTNFDKNLPNEKV
LPKHSLLYEYFTVYNELTKVKYVTEGMRKPAFLSGEQKKAIVDLLFKTNRKVTVKQLKE
DYFKKIECFDSVEISGVEDRFNASLGTYHDLLKIIKDKDFLDNEENEDILEDIVLTLTL
FEDREMIEERLKTYAHLFDDKVMKQLKRRRYTGWGRLSRKLINGIRDKQSGKTILDFLK
SDGFANRNFMQLIHDDSLTFKEDIQKAQVSGQGDSLHEHIANLAGSPAIKKGILQTVKV
VDELVKVMGRHKPENIVIEMARENQTTQKGQKNSRERMKRIEEGIKELGSQILKEHPVE
NTQLQNEKLYLYYLQNGRDMYVDQELDINRLSDYDVDHIVPQSFLKDDSIDNKVLTRSD
KNRGKSDNVPSEEVVKKMKNYWRQLLNAKLITQRKFDNLTKAERGGLSELDKAGFIKRQ
LVETRQITKHVAQILDSRMNTKYDENDKLIREVKVITLKSKLVSDFRKDFQFYKVREIN
NYHHAHDAYLNAVVGTALIKKYPKLESEFVYGDYKVYDVRKMIAKSEQEIGKATAKYFF
YSNIMNFFKTEITLANGEIRKRPLIETNGETGEIVWDKGRDFATVRKVLSMPQVNIVKK
TEVQTGGFSKESILPKRNSDKLIARKKDWDPKKYGGFDSPTVAYSVLVVAKVEKGKSKK
LKSVKELLGITIMERSSFEKNPIDFLEAKGYKEVKKDLIIKLPKYSLFELENGRKRMLA
SAGELQKGNELALPSKYVNFLYLASHYEKLKGSPEDNEQKQLFVEQHKHYLDEIIEQIS
EFSKRVILADANLDKVLSAYNKHRDKPIREQAENIIHLFTLTNLGAPAAFKYFDTTIDR
KRYTSTKEVLDATLIHQSITGLYETRIDLSQLGGDSGGSGGSGGSTNLSDIIEKETGKQ
LVIQESILMLPEEVEEVIGNKPESDILVHTAYDESTDENVMLLTSDAPEYKPWALVIQD
SNGENKIKMLSGGSGGSGGSTNLSDIIEKETGKQLVIQESILMLPEEVEEVIGNKPESD
ILVHTAYDESTDENVMLLTSDAPEYKPWALVIQDSNGENKIKMLSGGSPKKKRK
BE4-Gam: SEQ ID NO: 383
MAKPAKRIKSAAAAYVPQNRDAVITDIKRIGDLQREASRLETEMNDAIAEITEKFAARI
APIKTDIETLSKGVQGWCEANRDELTNGGKVKTANLVTGDVSWRVRPPSVSIRGMDAVM
ETLERLGLQRFIRTKQEINKEAILLEPKAVAGVAGITVKSGIEDFSIIPFEQEAGISGS
ETPGTSESATPESSSETGPVAVDPTLRRRIEPHEFEVFFDPRELRKETCLLYEINWGGR
HSIWRHTSQNTNKHVEVNFIEKFTTERYFCPNTRCSITWFLSWSPCGECSRAITEFLSR
YPHVTLFIYIARLYHHADPRNRQGLRDLISSGVTIQIMTEQESGYCWRNFVNYSPSNEA
HWPRYPHLWVRLYVLELYCIILGLPPCLNILRRKQPQLTFFTIALQSCHYQRLPPHILW
ATGLKSGGSSGGSSGSETPGTSESATPESSGGSSGGSDKKYSIGLAIGTNSVGWAVITD
EYKVPSKKFKVLGNTDRHSIKKNLIGALLFDSGETAEATRLKRTARRRYTRRKNRICYL
QEIFSNEMAKVDDSFFHRLEESFLVEEDKKHERHPIFGNIVDEVAYHEKYPTIYHLRKK
LVDSTDKADLRLIYLALAHMIKFRGHFLIEGDLNPDNSDVDKLFIQLVQTYNQLFEENP
INASGVDAKAILSARLSKSRRLENLIAQLPGEKKNGLFGNLIALSLGLTPNFKSNFDLA
EDAKLQLSKDTYDDDLDNLLAQIGDQYADLFLAAKNLSDAILLSDILRVNTEITKAPLS
ASMIKRYDEHHQDLTLLKALVRQQLPEKYKEIFFDQSKNGYAGYIDGGASQEEFYKFIK
PILEKMDGTEELLVKLNREDLLRKQRTFDNGSIPHQIHLGELHAILRRQEDFYPFLKDN
REKIEKILTFRIPYYVGPLARGNSRFAWMTRKSEETITPWNFEEVVDKGASAQSFIERM
TNFDKNLPNEKVLPKHSLLYEYFTVYNELTKVKYVTEGMRKPAFLSGEQKKAIVDLLFK
TNRKVTVKQLKEDYFKKIECFDSVEISGVEDRFNASLGTYHDLLKIIKDKDFLDNEENE
DILEDIVLTLTLFEDREMIEERLKTYAHLFDDKVMKQLKRRRYTGWGRLSRKLINGIRD
KQSGKTILDFLKSDGFANRNFMQLIHDDSLTFKEDIQKAQVSGQGDSLHEHIANLAGSP
AIKKGILQTVKVVDELVKVMGRHKPENIVIEMARENQTTQKGQKNSRERMKRIEEGIKE
LGSQILKEHPVENTQLQNEKLYLYYLQNGRDMYVDQELDINRLSDYDVDHIVPQSFLKD
DSIDNKVLTRSDKNRGKSDNVPSEEVVKKMKNYWRQLLNAKLITQRKFDNLTKAERGGL
SELDKAGFIKRQLVETRQITKHVAQILDSRMNTKYDENDKLIREVKVITLKSKLVSDFR
KDFQFYKVREINNYHHAHDAYLNAVVGTALIKKYPKLESEFVYGDYKVYDVRKMIAKSE
QEIGKATAKYFFYSNIMNFFKTEITLANGEIRKRPLIETNGETGEIVWDKGRDFATVRK
VLSMPQVNIVKKTEVQTGGFSKESILPKRNSDKLIARKKDWDPKKYGGFDSPTVAYSVL
VVAKVEKGKSKKLKSVKELLGITIMERSSFEKNPIDFLEAKGYKEVKKDLIIKLPKYSL
FELENGRKRMLASAGELQKGNELALPSKYVNFLYLASHYEKLKGSPEDNEQKQLFVEQH
KHYLDEIIEQISEFSKRVILADANLDKVLSAYNKHRDKPIREQAENIIHLFTLTNLGAP
AAFKYFDTTIDRKRYTSTKEVLDATLIHQSITGLYETRIDLSQLGGDSGGSGGSGGSTN
LSDIIEKETGKQLVIQESILMLPEEVEEVIGNKPESDILVHTAYDESTDENVMLLTSDA
PEYKPWALVIQDSNGENKIKMLSGGSGGSGGSTNLSDIIEKETGKQLVIQESILMLPEE
VEEVIGNKPESDILVHTAYDESTDENVMLLTSDAPEYKPWALVIQDSNGENKIKMLSGG
SPKKKRK
SaBE4: SEQ ID NO: 384
MSSETGPVAVDPTLRRRIEPHEFEVFFDPRELRKETCLLYEINWGGRHSIWRHTSQNTN
KHVEVNFIEKFTTERYFCPNTRCSITWFLSWSPCGECSRAITEFLSRYPHVTLFIYIAR
LYHHADPRNRQGLRDLISSGVTIQIMTEQESGYCWRNFVNYSPSNEAHWPRYPHLWVRL
YVLELYCIILGLPPCLNILRRKQPQLTFFTIALQSCHYQRLPPHILWATGLKSGGSSGG
SSGSETPGTSESATPESSGGSSGGSGKRNYILGLAIGITSVGYGIIDYETRDVIDAGVR
LFKEANVENNEGRRSKRGARRLKRRRRHRIQRVKKLLFDYNLLTDHSELSGINPYEARV
KGLSQKLSEEEFSAALLHLAKRRGVHNVNEVEEDTGNELSTKEQISRNSKALEEKYVAE
LQLERLKKDGEVRGSINRFKTSDYVKEAKQLLKVQKAYHQLDQSFIDTYIDLLETRRTY
YEGPGEGSPFGWKDIKEWYEMLMGHCTYFPEELRSVKYAYNADLYNALNDLNNLVITRD
ENEKLEYYEKFQIIENVFKQKKKPTLKQIAKEILVNEEDIKGYRVTSTGKPEFTNLKVY
HDIKDITARKEIIENAELLDQIAKILTIYQSSEDIQEELTNLNSELTQEEIEQISNLKG
YTGTHNLSLKAINLILDELWHTNDNQIAIFNRLKLVPKKVDLSQQKEIPTTLVDDFILS
PVVKRSFIQSIKVINAIIKKYGLPNDIIIELAREKNSKDAQKMINEMQKRNRQTNERIE
EIIRTTGKENAKYLIEKIKLHDMQEGKCLYSLEAIPLEDLLNNPFNYEVDHIIPRSVSF
DNSFNNKVLVKQEENSKKGNRTPFQYLSSSDSKISYETFKKHILNLAKGKGRISKTKKE
YLLEERDINRFSVQKDFINRNLVDTRYATRGLMNLLRSYFRVNNLDVKVKSINGGFTSF
LRRKWKFKKERNKGYKHHAEDALIIANADFIFKEWKKLDKAKKVMENQMFEEKQAESMP
EIETEQEYKEIFITPHQIKHIKDFKDYKYSHRVDKKPNRELINDTLYSTRKDDKGNTLI
VNNLNGLYDKDNDKLKKLINKSPEKLLMYHHDPQTYQKLKLIMEQYGDEKNPLYKYYEE
TGNYLTKYSKKDNGPVIKKIKYYGNKLNAHLDITDDYPNSRNKVVKLSLKPYRFDVYLD
NGVYKFVTVKNLDVIKKENYYEVNSKCYEEAKKLKKISNQAEFIASFYNNDLIKINGEL
YRVIGVNNDLLNRIEVNMIDITYREYLENMNDKRPPRIIKTIASKTQSIKKYSTDILGN
LYEVKSKKHPQIIKKGGSPKKKRKVSSDYKDHDGDYKDHDIDYKDDDDKSGGSGGSGGS
TNLSDIIEKETGKQLVIQESILMLPEEVEEVIGNKPESDILVHTAYDESTDENVMLLTS
DAPEYKPWALVIQDSNGENKIKMLSGGSGGSGGSTNLSDIIEKETGKQLVIQESILMLP
EEVEEVIGNKPESDILVHTAYDESTDENVMLLTSDAPEYKPWALVIQDSNGENKIKMLS
GGSPKKKRKV
SaBE4-Gam: SEQ ID NO: 385
MAKPAKRIKSAAAAYVPQNRDAVITDIKRIGDLQREASRLETEMNDAIAEITEKFAARI
APIKTDIETLSKGVQGWCEANRDELTNGGKVKTANLVTGDVSWRVRPPSVSIRGMDAVM
ETLERLGLQRFIRTKQEINKEAILLEPKAVAGVAGITVKSGIEDFSIIPFEQEAGISGS
ETPGTSESATPESSSETGPVAVDPTLRRRIEPHEFEVFFDPRELRKETCLLYEINWGGR
HSIWRHTSQNTNKHVEVNFIEKFTTERYFCPNTRCSITWFLSWSPCGECSRAITEFLSR
YPHVTLFIYIARLYHHADPRNRQGLRDLISSGVTIQIMTEQESGYCWRNFVNYSPSNEA
HWPRYPHLWVRLYVLELYCIILGLPPCLNILRRKQPQLTFFTIALQSCHYQRLPPHILW
ATGLKSGGSSGGSSGSETPGTSESATPESSGGSSGGSGKRNYILGLAIGITSVGYGIID
YETRDVIDAGVRLFKEANVENNEGRRSKRGARRLKRRRRHRIQRVKKLLFDYNLLTDHS
ELSGINPYEARVKGLSQKLSEEEFSAALLHLAKRRGVHNVNEVEEDTGNELSTKEQISR
NSKALEEKYVAELQLERLKKDGEVRGSINRFKTSDYVKEAKQLLKVQKAYHQLDQSFID
TYIDLLETRRTYYEGPGEGSPFGWKDIKEWYEMLMGHCTYFPEELRSVKYAYNADLYNA
LNDLNNLVITRDENEKLEYYEKFQIIENVFKQKKKPTLKQIAKEILVNEEDIKGYRVTS
TGKPEFTNLKVYHDIKDITARKEIIENAELLDQIAKILTIYQSSEDIQEELTNLNSELT
QEEIEQISNLKGYTGTHNLSLKAINLILDELWHTNDNQIAIFNRLKLVPKKVDLSQQKE
IPTTLVDDFILSPVVKRSFIQSIKVINAIIKKYGLPNDIIIELAREKNSKDAQKMINEM
QKRNRQTNERIEEIIRTTGKENAKYLIEKIKLHDMQEGKCLYSLEAIPLEDLLNNPFNY
EVDHIIPRSVSFDNSFNNKVLVKQEENSKKGNRTPFQYLSSSDSKISYETFKKHILNLA
KGKGRISKTKKEYLLEERDINRFSVQKDFINRNLVDTRYATRGLMNLLRSYFRVNNLDV
KVKSINGGFTSFLRRKWKFKKERNKGYKHHAEDALIIANADFIFKEWKKLDKAKKVMEN
QMFEEKQAESMPEIETEQEYKEIFITPHQIKHIKDFKDYKYSHRVDKKPNRELINDTLY
STRKDDKGNTLIVNNLNGLYDKDNDKLKKLINKSPEKLLMYHHDPQTYQKLKLIMEQYG
DEKNPLYKYYEETGNYLTKYSKKDNGPVIKKIKYYGNKLNAHLDITDDYPNSRNKVVKL
SLKPYRFDVYLDNGVYKFVTVKNLDVIKKENYYEVNSKCYEEAKKLKKISNQAEFIASF
YNNDLIKINGELYRVIGVNNDLLNRIEVNMIDITYREYLENMNDKRPPRIIKTIASKTQ
SIKKYSTDILGNLYEVKSKKHPQIIKKGGSPKKKRKVSSDYKDHDGDYKDHDIDYKDDD
DKSGGSGGSGGSTNLSDIIEKETGKQLVIQESILMLPEEVEEVIGNKPESDILVHTAYD
ESTDENVMLLTSDAPEYKPWALVIQDSNGENKIKMLSGGSGGSGGSTNLSDIIEKETGK
QLVIQESILMLPEEVEEVIGNKPESDILVHTAYDESTDENVMLLTSDAPEYKPWALVIQ
DSNGENKIKMLSGGSPKKKRKV
BE4max and AncBE4max, SEQ ID NO: 386
MKRTADGSEFESPKKKRKV [rat APOBEC1 or evoAPOBEC1, or FERNY,
or evoFERNY, or ancestral(anc) APOBEC, sequences see below]
SGGSSGGSSGSETPGTSESATPESSGGSSGGSDKKYSIGLAIGTNSVGWAVITDEYKVP
SKKFKVLGNTDRHSIKKNLIGALLFDSGETAEATRLKRTARRRYTRRKNRICYLQEIFS
NEMAKVDDSFFHRLEESFLVEEDKKHERHPIFGNIVDEVAYHEKYPTIYHLRKKLVDST
DKADLRLIYLALAHMIKFRGHFLIEGDLNPDNSDVDKLFIQLVQTYNQLFEENPINASG
VDAKAILSARLSKSRRLENLIAQLPGEKKNGLFGNLIALSLGLTPNFKSNFDLAEDAKL
QLSKDTYDDDLDNLLAQIGDQYADLFLAAKNLSDAILLSDILRVNTEITKAPLSASMIK
RYDEHHQDLTLLKALVRQQLPEKYKEIFFDQSKNGYAGYIDGGASQEEFYKFIKPILEK
MDGTEELLVKLNREDLLRKQRTFDNGSIPHQIHLGELHAILRRQEDFYPFLKDNREKIE
KILTFRIPYYVGPLARGNSRFAWMTRKSEETITPWNFEEVVDKGASAQSFIERMTNFDK
NLPNEKVLPKHSLLYEYFTVYNELTKVKYVTEGMRKPAFLSGEQKKAIVDLLFKTNRKV
TVKQLKEDYFKKIECFDSVEISGVEDRFNASLGTYHDLLKIIKDKDFLDNEENEDILED
IVLTLTLFEDREMIEERLKTYAHLFDDKVMKQLKRRRYTGWGRLSRKLINGIRDKQSGK
TILDFLKSDGFANRNFMQLIHDDSLTFKEDIQKAQVSGQGDSLHEHIANLAGSPAIKKG
ILQTVKVVDELVKVMGRHKPENIVIEMARENQTTQKGQKNSRERMKRIEEGIKELGSQI
LKEHPVENTQLQNEKLYLYYLQNGRDMYVDQELDINRLSDYDVDHIVPQSFLKDDSIDN
KVLTRSDKNRGKSDNVPSEEVVKKMKNYWRQLLNAKLITQRKFDNLTKAERGGLSELDK
AGFIKRQLVETRQITKHVAQILDSRMNTKYDENDKLIREVKVITLKSKLVSDFRKDFQF
YKVREINNYHHAHDAYLNAVVGTALIKKYPKLESEFVYGDYKVYDVRKMIAKSEQEIGK
ATAKYFFYSNIMNFFKTEITLANGEIRKRPLIETNGETGEIVWDKGRDFATVRKVLSMP
QVNIVKKTEVQTGGFSKESILPKRNSDKLIARKKDWDPKKYGGFDSPTVAYSVLVVAKV
EKGKSKKLKSVKELLGITIMERSSFEKNPIDFLEAKGYKEVKKDLIIKLPKYSLFELEN
GRKRMLASAGELQKGNELALPSKYVNFLYLASHYEKLKGSPEDNEQKQLFVEQHKHYLD
EIIEQISEFSKRVILADANLDKVLSAYNKHRDKPIREQAENIIHLFTLTNLGAPAAFKY
FDTTIDRKRYTSTKEVLDATLIHQSITGLYETRIDLSQLGGDSGGSGGSGGSTNLSDII
EKETGKQLVIQESILMLPEEVEEVIGNKPESDILVHTAYDESTDENVMLLTSDAPEYKP
WALVIQDSNGENKIKML_SGGSGGSGGS_TNLSDIIEKETGKQLVIQESILMLPEEVEE
VIGNKPESDILVHTAYDESTDENVMLLTSDAPEYKPWALVIQDSNGENKIKMLSGGSKR
TADGSEFEPKKKRKV
Rat APOBEC1, SEQ ID NO: 387
SSETGPVAVDPTLRRRIEPHEFEVFFDPRELRKETCLLYEINWGGRHSIWRHTSQNTNK
HVEVNFIEKFTTERYFCPNTRCSITWFLSWSPCGECSRAITEFLSRYPHVTLFIYIARL
YHHADPRNRQGLRDLISSGVTIQIMTEQESGYCWRNFVNYSPSNEAHWPRYPHLWVRLY
VLELYCIILGLPPCLNILRRKQPQLTFFTIALQSCHYQRLPPHILWATGLK
Anc689 APOBEC, SEQ ID NO: 388
SSETGPVAVDPTLRRRIEPHEFEVFFDPRELRKETCLLYEIKWGTSHKIWRHSSKNTTK
HVEVNFIEKFTSERHFCPSTSCSITWFLSWSPCGECSKAITEFLSQHPNVTLVIYVARL
YHHMDQQNRQGLRDLVNSGVTIQIMTAPEYDYCWRNFVNYPPGKEAHWPRYPPLWMKLY
ALELHAGILGLPPCLNILRRKQPQLTFFTIALQSCHYQRLPPHILWATGLK
Anc687 APOBEC, SEQ ID NO: 389
SSETGPVAVDPTLRRRIEPHEFEVFFDPRELRKEACLLYEIKWGTSHKIWRNSGKNTTK
HVEVNFIEKFTSERHFCPSISCSITWFLSWSPCWECSKAIREFLSQHPNVTLVIYVARL
FQHMDQQNRQGLRDLVNSGVTIQIMTASEYDHCWRNFVNYPPGKEAHWPRYPPLWMKLY
ALELHAGILGLPPCLNILRRKQPQLTFFTIALQSCHYQRLPPHILWATGLK
Anc686 APOBEC, SEQ ID NO: 390
SSETGPVAVDPTLRRRFEPEFFNRNYDPRELRKETYLLYEIKWGKESKIWRHTSNNRTQ
HAEVNFLENFFNELYFNPSTHCSITWFLSWSPCGECSKAIVEFLKEHPNVNLEIYVARL
YLCEDERNRQGLRDLVNSGVTIRIMNLPDYNYCWRTFVSHQGGDEDYWPRHFAPWVRLY
VLELYCIILGLPPCLNILRRKQPQLTFFTIALQSCHYQRLPPHILWATGLK
Anc655 APOBEC, SEQ ID NO: 391
SSETGPVAVDPTLRRRIEPFYFQFNNDPRACRRKTYLCYELKQDGSTWVWKRTLHNKGR
HAEICFLEKISSLEKLDPAQHYRITWYMSWSPCSNCAQKIVDFLKEHPHVNLRIYVARL
YYHEEERYQEGLRNLRRSGVSIRVMDLPDFEHCWETFVDNGGGPFQPWPGLEELNSKQL
SRRLQAGILGLPPCLNILRRKQPQLTFFTIALQSCHYQRLPPHILWATGLK
Anc733 APOBEC, SEQ ID NO: 392
SSETGPVAVDPTLRRRIEPFHFQFNNDPRAYRRKTYLCYELKQDGSTWVLDRTLRNKGR
HAEICFLDKINSWERLDPAQHYRVTWYMSWSPCSNCAQQVVDFLKEHPHVNLRIFAARL
YYHEQRRYQEGLRSLRGSGVPVAVMTLPDFEHCWETFVDHGGRPFQPWDGLEELNSRSL
SRRLQAGILGLPPCLNILRRKQPQLTFFTIALQSCHYQRLPPHILWATGLK
APOBEC ancestor #686, SEQ ID NO: 393
EFFNRNYDPRELRKETYLLYEIKWGKESKIWRHWCTSNNRTQHAEVNFLENFFNELYFN
PSTHCSITWFLSWSPCGECSKAIVEFLKEHPNVNLEIYVARLYLCEDERNRQGLRDLVN
SGVTIRIMNLPDYNYCWRTFVSHQGGDEDYWPRHFAPWVRL
APOBEC ancestor #733, SEQ ID NO: 394
FHFQFNNDPRAYRRKTYLCYELKQDGSTWVLDRGCTLRNKGRHAEICFLDKINSWERLD
PAQHYRVTWYMSWSPCSNCAQQVVDFLKEHPHVNLRIFAARLYYHEQRRYQEGLRSLRG
SGVPVAVMTLPDFEHCWETFVDHGGRPFQPWDGLEELNSRSLSRRLQAG
APOBEC ancestor #656_FERNY, SEQ ID NO: 395
FERNYDPRELRKETYLLYEIKWGKSGKLWRHWCQNNRTQHAEVYFLENIFNARRFNPST
HCSITWYLSWSPCAECSQKIVDFLKEHPNVNLEIYVARLYYHEDERNRQGLRDLVNSGV
TIRIMDLPDYNYCWKTFVSDQGGDEDYWPGHFAPWIKQYSLKL
APOBEC ancestor #655, SEQ ID NO: 396
FYFQFNNDPRACRRKTYLCYELKQDGSTWVWKRGCTLHNKGRHAEICFLEKISSLEKLD
PAQHYRITWYMSWSPCSNCAQKIVDFLKEHPHVNLRIYVARLYYHEEERYQEGLRNLRR
SGVSIRVMDLPDFEHCWETFVDNGGGPFQPWPGLEENSKQLSRRLQAG
APOBEC ancestor #649, SEQ ID NO: 397
FYEEFNNTLKSCRHKTLLCFSLKQDENTTLWKWGYAHNNGRHAEILVLREIENYEKLDP
AAKYRITLYMSYSPCNDCADKIVDFLKKHPNVNLNIKVSRLYYHEDEKYQEGLRNLKQP
GVSLKVMDRSDFEECFDLFVDPGGGEFQPWPGLEEKSKQYSATLQAG
evoFERNY-APOBEC1, SEQ ID NO: 398
SFERNYDPRELRKETYLLYEIKWGKSGKLWRHWCQNNRTQHAEVYFLENIFNARRFNPS
THCSITWYLSWSPCAECSQKIVDFLKEHPNVNLEIYVARLYYPENERNRQGLRDLVNSG
VTIRIMDLPDYNYCWKTFVSDQGGDEDYWPGHFAPWIKQYSLKL
evoAPOBEC1, SEQ ID NO: 399
SSKTGPVAVDPTLRRRIEPHEFEVFFDPRELRKETCLLYEINWGGRHSIWRHTSQNTNK
HVEVNFIEKFTTERYFCPNTRCSITWFLSWSPCGECSRAITEFLSRYPNVTLFIYIARL
YHLANPRNRQGLRDLISSGVTIQIMTEQESGYCWHNFVNYSPSNESHWPRYPHLWVRLY
VLELYCIILGLPPCLNILRRKQSQLTSFTIALQSCHYQRLPPHILWATGLK
ABE6.3, SEQ ID NO: 400
MSEVEFSHEYWMRHALTLAKRAWDEREVPVGAVLVHNNRVIGEGWNRPIGRHDPTAHAE
IMALRQGGLVMQNYRLIDATYVTLEPCVMCAGAMIHSRIGRVVFGARDAKTGAAGSLMD
VLHHPGMNHRVEITEGILADECAALLSDFFRMRRQEIKAQKKAQSSTDSGGSSGGSSGS
ETPGTSESATPESSGGSSGGSSEVEFSHEYWMRHALTLAKRAWDEREVPVGAVLVLNNR
VIGEGWNRSIGLHDPTAHAEIMALRQGGLVMQNYRLIDATLYVTFEPCVMCAGAMIHSR
IGRVVFGVRNAKTGAAGSLMDVLHYPGMNHRVEITEGILADECAALLCYFFRMRRQVFN
AQKKAQSSTDSGGSSGGSSGSETPGTSESATPESSGGSSGGSDKKYSIGLAIGTNSVGW
AVITDEYKVPSKKFKVLGNTDRHSIKKNLIGALLFDSGETAEATRLKRTARRRYTRRKN
RICYLQEIFSNEMAKVDDSFFHRLEESFLVEEDKKHERHPIFGNIVDEVAYHEKYPTIY
HLRKKLVDSTDKADLRLIYLALAHMIKFRGHFLIEGDLNPDNSDVDKLFIQLVQTYNQL
FEENPINASGVDAKAILSARLSKSRRLENLIAQLPGEKKNGLFGNLIALSLGLTPNFKS
NFDLAEDAKLQLSKDTYDDDLDNLLAQIGDQYADLFLAAKNLSDAILLSDILRVNTEIT
KAPLSASMIKRYDEHHQDLTLLKALVRQQLPEKYKEIFFDQSKNGYAGYIDGGASQEEF
YKFIKPILEKMDGTEELLVKLNREDLLRKQRTFDNGSIPHQIHLGELHAILRRQEDFYP
FLKDNREKIEKILTFRIPYYVGPLARGNSRFAWMTRKSEETITPWNFEEVVDKGASAQS
FIERMTNFDKNLPNEKVLPKHSLLYEYFTVYNELTKVKYVTEGMRKPAFLSGEQKKAIV
DLLFKTNRKVTVKQLKEDYFKKIECFDSVEISGVEDRFNASLGTYHDLLKIIKDKDFLD
NEENEDILEDIVLTLTLFEDREMIEERLKTYAHLFDDKVMKQLKRRRYTGWGRLSRKLI
NGIRDKQSGKTILDFLKSDGFANRNFMQLIHDDSLTFKEDIQKAQVSGQGDSLHEHIAN
LAGSPAIKKGILQTVKVVDELVKVMGRHKPENIVIEMARENQTTQKGQKNSRERMKRIE
EGIKELGSQILKEHPVENTQLQNEKLYLYYLQNGRDMYVDQELDINRLSDYDVDHIVPQ
SFLKDDSIDNKVLTRSDKNRGKSDNVPSEEVVKKMKNYWRQLLNAKLITQRKFDNLTKA
ERGGLSELDKAGFIKRQLVETRQITKHVAQILDSRMNTKYDENDKLIREVKVITLKSKL
VSDFRKDFQFYKVREINNYHHAHDAYLNAVVGTALIKKYPKLESEFVYGDYKVYDVRKM
IAKSEQEIGKATAKYFFYSNIMNFFKTEITLANGEIRKRPLIETNGETGEIVWDKGRDF
ATVRKVLSMPQVNIVKKTEVQTGGFSKESILPKRNSDKLIARKKDWDPKKYGGFDSPTV
AYSVLVVAKVEKGKSKKLKSVKELLGITIMERSSFEKNPIDFLEAKGYKEVKKDLIIKL
PKYSLFELENGRKRMLASAGELQKGNELALPSKYVNFLYLASHYEKLKGSPEDNEQKQL
FVEQHKHYLDEIIEQISEFSKRVILADANLDKVLSAYNKHRDKPIREQAENIIHLFTLT
NLGAPAAFKYFDTTIDRKRYTSTKEVLDATLIHQSITGLYETRIDLSQLGGDSGGSPKK
KRKV
ABE7.8, SEQ ID NO: 401
MSEVEFSHEYWMRHALTLAKRAWDEREVPVGAVLVHNNRVIGEGWNRPIGRHDPTAHAE
IMALRQGGLVMQNYRLIDATLYVTLEPCVMCAGAMIHSRIGRVVFGARDAKTGAAGSLM
DVLHHPGMNHRVEITEGILADECAALLSDFFRMRRQEIKAQKKAQSSTDSGGSSGGSSG
SETPGTSESATPESSGGSSGGSSEVEFSHEYWMRHALTLAKRALDEREVPVGAVLVLNN
RVIGEGWNRAIGLHDPTAHAEIMALRQGGLVMQNYRLIDATLYVTFEPCVMCAGAMIHS
RIGRVVFGVRNAKTGAAGSLMDVLHYPGMNHRVEITEGILADECNALLCYFFRMRRQVF
NAQKKAQSSTDSGGSSGGSSGSETPGTSESATPESSGGSSGGSDKKYSIGLAIGTNSVG
WAVITDEYKVPSKKFKVLGNTDRHSIKKNLIGALLFDSGETAEATRLKRTARRRYTRRK
NRICYLQEIFSNEMAKVDDSFFHRLEESFLVEEDKKHERHPIFGNIVDEVAYHEKYPTI
YHLRKKLVDSTDKADLRLIYLALAHMIKFRGHFLIEGDLNPDNSDVDKLFIQLVQTYNQ
LFEENPINASGVDAKAILSARLSKSRRLENLIAQLPGEKKNGLFGNLIALSLGLTPNFK
SNFDLAEDAKLQLSKDTYDDDLDNLLAQIGDQYADLFLAAKNLSDAILLSDILRVNTEI
TKAPLSASMIKRYDEHHQDLTLLKALVRQQLPEKYKEIFFDQSKNGYAGYIDGGASQEE
FYKFIKPILEKMDGTEELLVKLNREDLLRKQRTFDNGSIPHQIHLGELHAILRRQEDFY
PFLKDNREKIEKILTFRIPYYVGPLARGNSRFAWMTRKSEETITPWNFEEVVDKGASAQ
SFIERMTNFDKNLPNEKVLPKHSLLYEYFTVYNELTKVKYVTEGMRKPAFLSGEQKKAI
VDLLFKTNRKVTVKQLKEDYFKKIECFDSVEISGVEDRFNASLGTYHDLLKIIKDKDFL
DNEENEDILEDIVLTLTLFEDREMIEERLKTYAHLFDDKVMKQLKRRRYTGWGRLSRKL
INGIRDKQSGKTILDFLKSDGFANRNFMQLIHDDSLTFKEDIQKAQVSGQGDSLHEHIA
NLAGSPAIKKGILQTVKVVDELVKVMGRHKPENIVIEMARENQTTQKGQKNSRERMKRI
EEGIKELGSQILKEHPVENTQLQNEKLYLYYLQNGRDMYVDQELDINRLSDYDVDHIVP
QSFLKDDSIDNKVLTRSDKNRGKSDNVPSEEVVKKMKNYWRQLLNAKLITQRKFDNLTK
AERGGLSELDKAGFIKRQLVETRQITKHVAQILDSRMNTKYDENDKLIREVKVITLKSK
LVSDFRKDFQFYKVREINNYHHAHDAYLNAVVGTALIKKYPKLESEFVYGDYKVYDVRK
MIAKSEQEIGKATAKYFFYSNIMNFFKTEITLANGEIRKRPLIETNGETGEIVWDKGRD
FATVRKVLSMPQVNIVKKTEVQTGGFSKESILPKRNSDKLIARKKDWDPKKYGGFDSPT
VAYSVLVVAKVEKGKSKKLKSVKELLGITIMERSSFEKNPIDFLEAKGYKEVKKDLIIK
LPKYSLFELENGRKRMLASAGELQKGNELALPSKYVNFLYLASHYEKLKGSPEDNEQKQ
LFVEQHKHYLDEIIEQISEFSKRVILADANLDKVLSAYNKHRDKPIREQAENIIHLFTL
TNLGAPAAFKYFDTTIDRKRYTSTKEVLDATLIHQSITGLYETRIDLSQLGGDSGGSPK
KKRKV
ABE7.9, SEQ ID NO: 402
MSEVEFSHEYWMRHALTLAKRAWDEREVPVGAVLVHNNRVIGEGWNRPIGRHDPTAHAE
IMALRQGGLVMQNYRLIDATLYVTLEPCVMCAGAMIHSRIGRVVFGARDAKTGAAGSLM
DVLHHPGMNHRVEITEGILADECAALLSDFFRMRRQEIKAQKKAQSSTDSGGSSGGSSG
SETPGTSESATPESSGGSSGGSSEVEFSHEYWMRHALTLAKRALDEREVPVGAVLVLNN
RVIGEGWNRAIGLHDPTAHAEIMALRQGGLVMQNYRLIDATLYVTFEPCVMCAGAMIHS
RIGRVVFGVRNAKTGAAGSLMDVLHYPGMNHRVEITEGILADECNALLCYFFRMPRQVF
NAQKKAQSSTDSGGSSGGSSGSETPGTSESATPESSGGSSGGSDKKYSIGLAIGTNSVG
WAVITDEYKVPSKKFKVLGNTDRHSIKKNLIGALLFDSGETAEATRLKRTARRRYTRRK
NRICYLQEIFSNEMAKVDDSFFHRLEESFLVEEDKKHERHPIFGNIVDEVAYHEKYPTI
YHLRKKLVDSTDKADLRLIYLALAHMIKFRGHFLIEGDLNPDNSDVDKLFIQLVQTYNQ
LFEENPINASGVDAKAILSARLSKSRRLENLIAQLPGEKKNGLFGNLIALSLGLTPNFK
SNFDLAEDAKLQLSKDTYDDDLDNLLAQIGDQYADLFLAAKNLSDAILLSDILRVNTEI
TKAPLSASMIKRYDEHHQDLTLLKALVRQQLPEKYKEIFFDQSKNGYAGYIDGGASQEE
FYKFIKPILEKMDGTEELLVKLNREDLLRKQRTFDNGSIPHQIHLGELHAILRRQEDFY
PFLKDNREKIEKILTFRIPYYVGPLARGNSRFAWMTRKSEETITPWNFEEVVDKGASAQ
SFIERMTNFDKNLPNEKVLPKHSLLYEYFTVYNELTKVKYVTEGMRKPAFLSGEQKKAI
VDLLFKTNRKVTVKQLKEDYFKKIECFDSVEISGVEDRFNASLGTYHDLLKIIKDKDFL
DNEENEDILEDIVLTLTLFEDREMIEERLKTYAHLFDDKVMKQLKRRRYTGWGRLSRKL
INGIRDKQSGKTILDFLKSDGFANRNFMQLIHDDSLTFKEDIQKAQVSGQGDSLHEHIA
NLAGSPAIKKGILQTVKVVDELVKVMGRHKPENIVIEMARENQTTQKGQKNSRERMKRI
EEGIKELGSQILKEHPVENTQLQNEKLYLYYLQNGRDMYVDQELDINRLSDYDVDHIVP
QSFLKDDSIDNKVLTRSDKNRGKSDNVPSEEVVKKMKNYWRQLLNAKLITQRKFDNLTK
AERGGLSELDKAGFIKRQLVETRQITKHVAQILDSRMNTKYDENDKLIREVKVITLKSK
LVSDFRKDFQFYKVREINNYHHAHDAYLNAVVGTALIKKYPKLESEFVYGDYKVYDVRK
MIAKSEQEIGKATAKYFFYSNIMNFFKTEITLANGEIRKRPLIETNGETGEIVWDKGRD
FATVRKVLSMPQVNIVKKTEVQTGGFSKESILPKRNSDKLIARKKDWDPKKYGGFDSPT
VAYSVLVVAKVEKGKSKKLKSVKELLGITIMERSSFEKNPIDFLEAKGYKEVKKDLIIK
LPKYSLFELENGRKRMLASAGELQKGNELALPSKYVNFLYLASHYEKLKGSPEDNEQKQ
LFVEQHKHYLDEIIEQISEFSKRVILADANLDKVLSAYNKHRDKPIREQAENIIHLFTL
TNLGAPAAFKYFDTTIDRKRYTSTKEVLDATLIHQSITGLYETRIDLSQLGGDSGGSPK
KKRKV
ABE7.10, SEQ ID NO: 403
MSEVEFSHEYWMRHALTLAKRAWDEREVPVGAVLVHNNRVIGEGWNRPIGRHDPTAHAE
IMALRQGGLVMQNYRLIDATLYVTLEPCVMCAGAMIHSRIGRVVFGARDAKTGAAGSLM
DVLHHPGMNHRVEITEGILADECAALLSDFFRMRRQEIKAQKKAQSSTDSGGSSGGSSG
SETPGTSESATPESSGGSSGGSSEVEFSHEYWMRHALTLAKRARDEREVPVGAVLVLNN
RVIGEGWNRAIGLHDPTAHAEIMALRQGGLVMQNYRLIDATLYVTFEPCVMCAGAMIHS
RIGRVVFGVRNAKTGAAGSLMDVLHYPGMNHRVEITEGILADECAALLCYFFRMPRQVF
NAQKKAQSSTDSGGSSGGSSGSETPGTSESATPESSGGSSGGSDKKYSIGLAIGTNSVG
WAVITDEYKVPSKKFKVLGNTDRHSIKKNLIGALLFDSGETAEATRLKRTARRRYTRRK
NRICYLQEIFSNEMAKVDDSFFHRLEESFLVEEDKKHERHPIFGNIVDEVAYHEKYPTI
YHLRKKLVDSTDKADLRLIYLALAHMIKFRGHFLIEGDLNPDNSDVDKLFIQLVQTYNQ
LFEENPINASGVDAKAILSARLSKSRRLENLIAQLPGEKKNGLFGNLIALSLGLTPNFK
SNFDLAEDAKLQLSKDTYDDDLDNLLAQIGDQYADLFLAAKNLSDAILLSDILRVNTEI
TKAPLSASMIKRYDEHHQDLTLLKALVRQQLPEKYKEIFFDQSKNGYAGYIDGGASQEE
FYKFIKPILEKMDGTEELLVKLNREDLLRKQRTFDNGSIPHQIHLGELHAILRRQEDFY
PFLKDNREKIEKILTFRIPYYVGPLARGNSRFAWMTRKSEETITPWNFEEVVDKGASAQ
SFIERMTNFDKNLPNEKVLPKHSLLYEYFTVYNELTKVKYVTEGMRKPAFLSGEQKKAI
VDLLFKTNRKVTVKQLKEDYFKKIECFDSVEISGVEDRFNASLGTYHDLLKIIKDKDFL
DNEENEDILEDIVLTLTLFEDREMIEERLKTYAHLFDDKVMKQLKRRRYTGWGRLSRKL
INGIRDKQSGKTILDFLKSDGFANRNFMQLIHDDSLTFKEDIQKAQVSGQGDSLHEHIA
NLAGSPAIKKGILQTVKVVDELVKVMGRHKPENIVIEMARENQTTQKGQKNSRERMKRI
EEGIKELGSQILKEHPVENTQLQNEKLYLYYLQNGRDMYVDQELDINRLSDYDVDHIVP
QSFLKDDSIDNKVLTRSDKNRGKSDNVPSEEVVKKMKNYWRQLLNAKLITQRKFDNLTK
AERGGLSELDKAGFIKRQLVETRQITKHVAQILDSRMNTKYDENDKLIREVKVITLKSK
LVSDFRKDFQFYKVREINNYHHAHDAYLNAVVGTALIKKYPKLESEFVYGDYKVYDVRK
MIAKSEQEIGKATAKYFFYSNIMNFFKTEITLANGEIRKRPLIETNGETGEIVWDKGRD
FATVRKVLSMPQVNIVKKTEVQTGGFSKESILPKRNSDKLIARKKDWDPKKYGGFDSPT
VAYSVLVVAKVEKGKSKKLKSVKELLGITIMERSSFEKNPIDFLEAKGYKEVKKDLIIK
LPKYSLFELENGRKRMLASAGELQKGNELALPSKYVNFLYLASHYEKLKGSPEDNEQKQ
LFVEQHKHYLDEIIEQISEFSKRVILADANLDKVLSAYNKHRDKPIREQAENIIHLFTL
TNLGAPAAFKYFDTTIDRKRYTSTKEVLDATLIHQSITGLYETRIDLSQLGGDSGGSPK
KKRKV
ABEmax, SEQ ID NO: 404
MKRTADGSEFESPKKKRKVSEVEFSHEYWMRHALTLAKRAWDEREVPVGAVLVHNNRVI
GEGWNRPIGRHDPTAHAEIMALRQGGLVMQNYRLIDATLYVTLEPCVMCAGAMIHSRIG
RVVFGARDAKTGAAGSLMDVLHHPGMNHRVEITEGILADECAALLSDFFRMRRQEIKAQ
KKAQSSTDSGGSSGGSSGSETPGTSESATPESSGGSSGGSSEVEFSHEYWMRHALTLAK
RARDEREVPVGAVLVLNNRVIGEGWNRAIGLHDPTAHAEIMALRQGGLVMQNYRLIDAT
LYVTFEPCVMCAGAMIHSRIGRVVFGVRNAKTGAAGSLMDVLHYPGMNHRVEITEGILA
DECAALLCYFFRMPRQVFNAQKKAQSSTDSGGSSGGSSGSETPGTSESATPESSGGSSG
GSDKKYSIGLAIGTNSVGWAVITDEYKVPSKKFKVLGNTDRHSIKKNLIGALLFDSGET
AEATRLKRTARRRYTRRKNRICYLQEIFSNEMAKVDDSFFHRLEESFLVEEDKKHERHP
IFGNIVDEVAYHEKYPTIYHLRKKLVDSTDKADLRLIYLALAHMIKFRGHFLIEGDLNP
DNSDVDKLFIQLVQTYNQLFEENPINASGVDAKAILSARLSKSRRLENLIAQLPGEKKN
GLFGNLIALSLGLTPNFKSNFDLAEDAKLQLSKDTYDDDLDNLLAQIGDQYADLFLAAK
NLSDAILLSDILRVNTEITKAPLSASMIKRYDEHHQDLTLLKALVRQQLPEKYKEIFFD
QSKNGYAGYIDGGASQEEFYKFIKPILEKMDGTEELLVKLNREDLLRKQRTFDNGSIPH
QIHLGELHAILRRQEDFYPFLKDNREKIEKILTFRIPYYVGPLARGNSRFAWMTRKSEE
TITPWNFEEVVDKGASAQSFIERMTNFDKNLPNEKVLPKHSLLYEYFTVYNELTKVKYV
TEGMRKPAFLSGEQKKAIVDLLFKTNRKVTVKQLKEDYFKKIECFDSVEISGVEDRFNA
SLGTYHDLLKIIKDKDFLDNEENEDILEDIVLTLTLFEDREMIEERLKTYAHLFDDKVM
KQLKRRRYTGWGRLSRKLINGIRDKQSGKTILDFLKSDGFANRNFMQLIHDDSLTFKED
IQKAQVSGQGDSLHEHIANLAGSPAIKKGILQTVKVVDELVKVMGRHKPENIVIEMARE
NQTTQKGQKNSRERMKRIEEGIKELGSQILKEHPVENTQLQNEKLYLYYLQNGRDMYVD
QELDINRLSDYDVDHIVPQSFLKDDSIDNKVLTRSDKNRGKSDNVPSEEVVKKMKNYWR
QLLNAKLITQRKFDNLTKAERGGLSELDKAGFIKRQLVETRQITKHVAQILDSRMNTKY
DENDKLIREVKVITLKSKLVSDFRKDFQFYKVREINNYHHAHDAYLNAVVGTALIKKYP
KLESEFVYGDYKVYDVRKMIAKSEQEIGKATAKYFFYSNIMNFFKTEITLANGEIRKRP
LIETNGETGEIVWDKGRDFATVRKVLSMPQVNIVKKTEVQTGGFSKESILPKRNSDKLI
ARKKDWDPKKYGGFDSPTVAYSVLVVAKVEKGKSKKLKSVKELLGITIMERSSFEKNPI
DFLEAKGYKEVKKDLIIKLPKYSLFELENGRKRMLASAGELQKGNELALPSKYVNFLYL
ASHYEKLKGSPEDNEQKQLFVEQHKHYLDEIIEQISEFSKRVILADANLDKVLSAYNKH
RDKPIREQAENIIHLFTLTNLGAPAAFKYFDTTIDRKRYTSTKEVLDATLIHQSITGLY
ETRIDLSQLGGDSGGSKRTADGSEFEPKKKRKV
miniABEmax, SEQ ID NO: 405
NLS-tadA(7.10)-32AA linker*-hSpCas9n(D10A)-NLS-P2A-EGFP-NLS
MKRTADGSEFESPKKKRKVSEVEFSHEYWMRHALTLAKRARDEREVPVGAVLVLNNRVI
GEGWNRAIGLHDPTAHAEIMALRQGGLVMQNYRLIDATLYVTFEPCVMCAGAMIHSRIG
RVVFGVRNAKTGAAGSLMDVLHYPGMNHRVEITEGILADECAALLCYFFRMPRQVFNAQ
KKAQSSTDSGGSSGGSSGSETPGTSESATPESSGGSSGGSDKKYSIGLAIGTNSVGWAV
ITDEYKVPSKKFKVLGNTDRHSIKKNLIGALLFDSGETAEATRLKRTARRRYTRRKNRI
CYLQEIFSNEMAKVDDSFFHRLEESFLVEEDKKHERHPIFGNIVDEVAYHEKYPTIYHL
RKKLVDSTDKADLRLIYLALAHMIKFRGHFLIEGDLNPDNSDVDKLFIQLVQTYNQLFE
ENPINASGVDAKAILSARLSKSRRLENLIAQLPGEKKNGLFGNLIALSLGLTPNFKSNF
DLAEDAKLQLSKDTYDDDLDNLLAQIGDQYADLFLAAKNLSDAILLSDILRVNTEITKA
PLSASMIKRYDEHHQDLTLLKALVRQQLPEKYKEIFFDQSKNGYAGYIDGGASQEEFYK
FIKPILEKMDGTEELLVKLNREDLLRKQRTFDNGSIPHQIHLGELHAILRRQEDFYPFL
KDNREKIEKILTFRIPYYVGPLARGNSRFAWMTRKSEETITPWNFEEVVDKGASAQSFI
ERMTNFDKNLPNEKVLPKHSLLYEYFTVYNELTKVKYVTEGMRKPAFLSGEQKKAIVDL
LFKTNRKVTVKQLKEDYFKKIECFDSVEISGVEDRFNASLGTYHDLLKIIKDKDFLDNE
ENEDILEDIVLTLTLFEDREMIEERLKTYAHLFDDKVMKQLKRRRYTGWGRLSRKLING
IRDKQSGKTILDFLKSDGFANRNFMQLIHDDSLTFKEDIQKAQVSGQGDSLHEHIANLA
GSPAIKKGILQTVKVVDELVKVMGRHKPENIVIEMARENQTTQKGQKNSRERMKRIEEG
IKELGSQILKEHPVENTQLQNEKLYLYYLQNGRDMYVDQELDINRLSDYDVDHIVPQSF
LKDDSIDNKVLTRSDKNRGKSDNVPSEEVVKKMKNYWRQLLNAKLITQRKFDNLTKAER
GGLSELDKAGFIKRQLVETRQITKHVAQILDSRMNTKYDENDKLIREVKVITLKSKLVS
DFRKDFQFYKVREINNYHHAHDAYLNAVVGTALIKKYPKLESEFVYGDYKVYDVRKMIA
KSEQEIGKATAKYFFYSNIMNFFKTEITLANGEIRKRPLIETNGETGEIVWDKGRDFAT
VRKVLSMPQVNIVKKTEVQTGGFSKESILPKRNSDKLIARKKDWDPKKYGGFDSPTVAY
SVLVVAKVEKGKSKKLKSVKELLGITIMERSSFEKNPIDFLEAKGYKEVKKDLIIKLPK
YSLFELENGRKRMLASAGELQKGNELALPSKYVNFLYLASHYEKLKGSPEDNEQKQLFV
EQHKHYLDEIIEQISEFSKRVILADANLDKVLSAYNKHRDKPIREQAENIIHLFTLTNL
GAPAAFKYFDTTIDRKRYTSTKEVLDATLIHQSITGLYETRIDLSQLGGDSGGSKRTAD
GSEFEPKKKRKVGSGATNFSLLKQAGDVEENPGPMVSKGEELFTGVVPILVELDGDVNG
HKFSVSGEGEGDATYGKLTLKFICTTGKLPVPWPTLVTTLTYGVQCFSRYPDHMKQHDF
FKSAMPEGYVQERTIFFKDDGNYKTRAEVKFEGDTLVNRIELKGIDFKEDGNILGHKLE
YNYNSHNVYIMADKQKNGIKVNFKIRHNIEDGSVQLADHYQQNTPIGDGPVLLPDNHYL
STQSALSKDPNEKRDHMVLLEFVTAAGITLGMDELYKSGGSPKKKRKV
miniABEmax-V82G, SEQ ID NO: 406:
MKRTADGSEFESPKKKRKVSEVEFSHEYWMRHALTLAKRARDEREVPVGAVLVLNNRVI
GEGWNRAIGLHDPTAHAEIMALRQGGLVMQNYRLIDATLYGTFEPCVMCAGAMIHSRIG
RVVFGVRNAKTGAAGSLMDVLHYPGMNHRVEITEGILADECAALLCYFFRMPRQVFNAQ
KKAQSSTDSGGSSGGSSGSETPGTSESATPESSGGSSGGSDKKYSIGLAIGTNSVGWAV
ITDEYKVPSKKFKVLGNTDRHSIKKNLIGALLFDSGETAEATRLKRTARRRYTRRKNRI
CYLQEIFSNEMAKVDDSFFHRLEESFLVEEDKKHERHPIFGNIVDEVAYHEKYPTIYHL
RKKLVDSTDKADLRLIYLALAHMIKFRGHFLIEGDLNPDNSDVDKLFIQLVQTYNQLFE
ENPINASGVDAKAILSARLSKSRRLENLIAQLPGEKKNGLFGNLIALSLGLTPNFKSNF
DLAEDAKLQLSKDTYDDDLDNLLAQIGDQYADLFLAAKNLSDAILLSDILRVNTEITKA
PLSASMIKRYDEHHQDLTLLKALVRQQLPEKYKEIFFDQSKNGYAGYIDGGASQEEFYK
FIKPILEKMDGTEELLVKLNREDLLRKQRTFDNGSIPHQIHLGELHAILRRQEDFYPFL
KDNREKIEKILTFRIPYYVGPLARGNSRFAWMTRKSEETITPWNFEEVVDKGASAQSFI
ERMTNFDKNLPNEKVLPKHSLLYEYFTVYNELTKVKYVTEGMRKPAFLSGEQKKAIVDL
LFKTNRKVTVKQLKEDYFKKIECFDSVEISGVEDRFNASLGTYHDLLKIIKDKDFLDNE
ENEDILEDIVLTLTLFEDREMIEERLKTYAHLFDDKVMKQLKRRRYTGWGRLSRKLING
IRDKQSGKTILDFLKSDGFANRNFMQLIHDDSLTFKEDIQKAQVSGQGDSLHEHIANLA
GSPAIKKGILQTVKVVDELVKVMGRHKPENIVIEMARENQTTQKGQKNSRERMKRIEEG
IKELGSQILKEHPVENTQLQNEKLYLYYLQNGRDMYVDQELDINRLSDYDVDHIVPQSF
LKDDSIDNKVLTRSDKNRGKSDNVPSEEVVKKMKNYWRQLLNAKLITQRKFDNLTKAER
GGLSELDKAGFIKRQLVETRQITKHVAQILDSRMNTKYDENDKLIREVKVITLKSKLVS
DFRKDFQFYKVREINNYHHAHDAYLNAVVGTALIKKYPKLESEFVYGDYKVYDVRKMIA
KSEQEIGKATAKYFFYSNIMNFFKTEITLANGEIRKRPLIETNGETGEIVWDKGRDFAT
VRKVLSMPQVNIVKKTEVQTGGFSKESILPKRNSDKLIARKKDWDPKKYGGFDSPTVAY
SVLVVAKVEKGKSKKLKSVKELLGITIMERSSFEKNPIDFLEAKGYKEVKKDLIIKLPK
YSLFELENGRKRMLASAGELQKGNELALPSKYVNFLYLASHYEKLKGSPEDNEQKQLFV
EQHKHYLDEIIEQISEFSKRVILADANLDKVLSAYNKHRDKPIREQAENIIHLFTLTNL
GAPAAFKYFDTTIDRKRYTSTKEVLDATLIHQSITGLYETRIDLSQLGGDSGGSKRTAD
GSEFEPKKKRKVGSGATNFSLLKQAGDVEENPGPMVSKGEELFTGVVPILVELDGDVNG
HKFSVSGEGEGDATYGKLTLKFICTTGKLPVPWPTLVTTLTYGVQCFSRYPDHMKQHDF
FKSAMPEGYVQERTIFFKDDGNYKTRAEVKFEGDTLVNRIELKGIDFKEDGNILGHKLE
YNYNSHNVYIMADKQKNGIKVNFKIRHNIEDGSVQLADHYQQNTPIGDGPVLLPDNHYL
STQSALSKDPNEKRDHMVLLEFVTAAGITLGMDELYKSGGSPKKKRKV
miniABEmax-K20A/R21A, SEQ ID NO: 407:
MKRTADGSEFESPKKKRKVSEVEFSHEYWMRHALTLAAAARDEREVPVGAVLVLNNRVI
GEGWNRAIGLHDPTAHAEIMALRQGGLVMQNYRLIDATLYVTFEPCVMCAGAMIHSRIG
RVVFGVRNAKTGAAGSLMDVLHYPGMNHRVEITEGILADECAALLCYFFRMPRQVFNAQ
KKAQSSTDSGGSSGGSSGSETPGTSESATPESSGGSSGGSDKKYSIGLAIGTNSVGWAV
ITDEYKVPSKKFKVLGNTDRHSIKKNLIGALLFDSGETAEATRLKRTARRRYTRRKNRI
CYLQEIFSNEMAKVDDSFFHRLEESFLVEEDKKHERHPIFGNIVDEVAYHEKYPTIYHL
RKKLVDSTDKADLRLIYLALAHMIKFRGHFLIEGDLNPDNSDVDKLFIQLVQTYNQLFE
ENPINASGVDAKAILSARLSKSRRLENLIAQLPGEKKNGLFGNLIALSLGLTPNFKSNF
DLAEDAKLQLSKDTYDDDLDNLLAQIGDQYADLFLAAKNLSDAILLSDILRVNTEITKA
PLSASMIKRYDEHHQDLTLLKALVRQQLPEKYKEIFFDQSKNGYAGYIDGGASQEEFYK
FIKPILEKMDGTEELLVKLNREDLLRKQRTFDNGSIPHQIHLGELHAILRRQEDFYPFL
KDNREKIEKILTFRIPYYVGPLARGNSRFAWMTRKSEETITPWNFEEVVDKGASAQSFI
ERMTNFDKNLPNEKVLPKHSLLYEYFTVYNELTKVKYVTEGMRKPAFLSGEQKKAIVDL
LFKTNRKVTVKQLKEDYFKKIECFDSVEISGVEDRFNASLGTYHDLLKIIKDKDFLDNE
ENEDILEDIVLTLTLFEDREMIEERLKTYAHLFDDKVMKQLKRRRYTGWGRLSRKLING
IRDKQSGKTILDFLKSDGFANRNFMQLIHDDSLTFKEDIQKAQVSGQGDSLHEHIANLA
GSPAIKKGILQTVKVVDELVKVMGRHKPENIVIEMARENQTTQKGQKNSRERMKRIEEG
IKELGSQILKEHPVENTQLQNEKLYLYYLQNGRDMYVDQELDINRLSDYDVDHIVPQSF
LKDDSIDNKVLTRSDKNRGKSDNVPSEEVVKKMKNYWRQLLNAKLITQRKFDNLTKAER
GGLSELDKAGFIKRQLVETRQITKHVAQILDSRMNTKYDENDKLIREVKVITLKSKLVS
DFRKDFQFYKVREINNYHHAHDAYLNAVVGTALIKKYPKLESEFVYGDYKVYDVRKMIA
KSEQEIGKATAKYFFYSNIMNFFKTEITLANGEIRKRPLIETNGETGEIVWDKGRDFAT
VRKVLSMPQVNIVKKTEVQTGGFSKESILPKRNSDKLIARKKDWDPKKYGGFDSPTVAY
SVLVVAKVEKGKSKKLKSVKELLGITIMERSSFEKNPIDFLEAKGYKEVKKDLIIKLPK
YSLFELENGRKRMLASAGELQKGNELALPSKYVNFLYLASHYEKLKGSPEDNEQKQLFV
EQHKHYLDEIIEQISEFSKRVILADANLDKVLSAYNKHRDKPIREQAENIIHLFTLTNL
GAPAAFKYFDTTIDRKRYTSTKEVLDATLIHQSITGLYETRIDLSQLGGDSGGSKRTAD
GSEFEPKKKRKVGSGATNFSLLKQAGDVEENPGPMVSKGEELFTGVVPILVELDGDVNG
HKFSVSGEGEGDATYGKLTLKFICTTGKLPVPWPTLVTTLTYGVQCFSRYPDHMKQHDF
FKSAMPEGYVQERTIFFKDDGNYKTRAEVKFEGDTLVNRIELKGIDFKEDGNILGHKLE
YNYNSHNVYIMADKQKNGIKVNFKIRHNIEDGSVQLADHYQQNTPIGDGPVLLPDNHYL
STQSALSKDPNEKRDHMVLLEFVTAAGITLGMDELYKSGGSPKKKRKV
miniABEmax-V106W, SEQ ID NO: 408:
MKRTADGSEFESPKKKRKVSEVEFSHEYWMRHALTLAKRARDEREVPVGAVLVLNNRVI
GEGWNRAIGLHDPTAHAEIMALRQGGLVMQNYRLIDATLYVTFEPCVMCAGAMIHSRIG
RVVFGWRNAKTGAAGSLMDVLHYPGMNHRVEITEGILADECAALLCYFFRMPRQVFNAQ
KKAQSSTDSGGSSGGSSGSETPGTSESATPESSGGSSGGSDKKYSIGLAIGTNSVGWAV
ITDEYKVPSKKFKVLGNTDRHSIKKNLIGALLFDSGETAEATRLKRTARRRYTRRKNRI
CYLQEIFSNEMAKVDDSFFHRLEESFLVEEDKKHERHPIFGNIVDEVAYHEKYPTIYHL
RKKLVDSTDKADLRLIYLALAHMIKFRGHFLIEGDLNPDNSDVDKLFIQLVQTYNQLFE
ENPINASGVDAKAILSARLSKSRRLENLIAQLPGEKKNGLFGNLIALSLGLTPNFKSNF
DLAEDAKLQLSKDTYDDDLDNLLAQIGDQYADLFLAAKNLSDAILLSDILRVNTEITKA
PLSASMIKRYDEHHQDLTLLKALVRQQLPEKYKEIFFDQSKNGYAGYIDGGASQEEFYK
FIKPILEKMDGTEELLVKLNREDLLRKQRTFDNGSIPHQIHLGELHAILRRQEDFYPFL
KDNREKIEKILTFRIPYYVGPLARGNSRFAWMTRKSEETITPWNFEEVVDKGASAQSFI
ERMTNFDKNLPNEKVLPKHSLLYEYFTVYNELTKVKYVTEGMRKPAFLSGEQKKAIVDL
LFKTNRKVTVKQLKEDYFKKIECFDSVEISGVEDRFNASLGTYHDLLKIIKDKDFLDNE
ENEDILEDIVLTLTLFEDREMIEERLKTYAHLFDDKVMKQLKRRRYTGWGRLSRKLING
IRDKQSGKTILDFLKSDGFANRNFMQLIHDDSLTFKEDIQKAQVSGQGDSLHEHIANLA
GSPAIKKGILQTVKVVDELVKVMGRHKPENIVIEMARENQTTQKGQKNSRERMKRIEEG
IKELGSQILKEHPVENTQLQNEKLYLYYLQNGRDMYVDQELDINRLSDYDVDHIVPQSF
LKDDSIDNKVLTRSDKNRGKSDNVPSEEVVKKMKNYWRQLLNAKLITQRKFDNLTKAER
GGLSELDKAGFIKRQLVETRQITKHVAQILDSRMNTKYDENDKLIREVKVITLKSKLVS
DFRKDFQFYKVREINNYHHAHDAYLNAVVGTALIKKYPKLESEFVYGDYKVYDVRKMIA
KSEQEIGKATAKYFFYSNIMNFFKTEITLANGEIRKRPLIETNGETGEIVWDKGRDFAT
VRKVLSMPQVNIVKKTEVQTGGFSKESILPKRNSDKLIARKKDWDPKKYGGFDSPTVAY
SVLVVAKVEKGKSKKLKSVKELLGITIMERSSFEKNPIDFLEAKGYKEVKKDLIIKLPK
YSLFELENGRKRMLASAGELQKGNELALPSKYVNFLYLASHYEKLKGSPEDNEQKQLFV
EQHKHYLDEIIEQISEFSKRVILADANLDKVLSAYNKHRDKPIREQAENIIHLFTLTNL
GAPAAFKYFDTTIDRKRYTSTKEVLDATLIHQSITGLYETRIDLSQLGGDSGGSKRTAD
GSEFEPKKKRKVGSGATNFSLLKQAGDVEENPGPMVSKGEELFTGVVPILVELDGDVNG
HKFSVSGEGEGDATYGKLTLKFICTTGKLPVPWPTLVTTLTYGVQCFSRYPDHMKQHDF
FKSAMPEGYVQERTIFFKDDGNYKTRAEVKFEGDTLVNRIELKGIDFKEDGNILGHKLE
YNYNSHNVYIMADKQKNGIKVNFKIRHNIEDGSVQLADHYQQNTPIGDGPVLLPDNHYL
STQSALSKDPNEKRDHMVLLEFVTAAGITLGMDELYKSGGSPKKKRKV
ABEmax containing newer ABE8 TadA* variants
(see below), SEQ ID NO: 409
MKRTADGSEFESPKKKRKVSEVEFSHEYWMRHALTLAKRAWDEREVPVGAVLVHNNRVI
GEGWNRPIGRHDPTAHAEIMALRQGGLVMQNYRLIDATLYVTLEPCVMCAGAMIHSRIG
RVVFGARDAKTGAAGSLMDVLHHPGMNHRVEITEGILADECAALLSDFFRMRRQEIKAQ
KKAQSSTDSGGSSGGSSGSETPGTSESATPESSGGSSGGS[TadA* ABE8e or
other ABE8 sequences as indicated below]SGGSSGGSSGSETPGTSES
ATPESSGGSSGGSDKKYSIGLAIGTNSVGWAVITDEYKVPSKKFKVLGNTDRHSIKKNL
IGALLFDSGETAEATRLKRTARRRYTRRKNRICYLQEIFSNEMAKVDDSFFHRLEESFL
VEEDKKHERHPIFGNIVDEVAYHEKYPTIYHLRKKLVDSTDKADLRLIYLALAHMIKFR
GHFLIEGDLNPDNSDVDKLFIQLVQTYNQLFEENPINASGVDAKAILSARLSKSRRLEN
LIAQLPGEKKNGLFGNLIALSLGLTPNFKSNFDLAEDAKLQLSKDTYDDDLDNLLAQIG
DQYADLFLAAKNLSDAILLSDILRVNTEITKAPLSASMIKRYDEHHQDLTLLKALVRQQ
LPEKYKEIFFDQSKNGYAGYIDGGASQEEFYKFIKPILEKMDGTEELLVKLNREDLLRK
QRTFDNGSIPHQIHLGELHAILRRQEDFYPFLKDNREKIEKILTFRIPYYVGPLARGNS
RFAWMTRKSEETITPWNFEEVVDKGASAQSFIERMTNFDKNLPNEKVLPKHSLLYEYFT
VYNELTKVKYVTEGMRKPAFLSGEQKKAIVDLLFKTNRKVTVKQLKEDYFKKIECFDSV
EISGVEDRFNASLGTYHDLLKIIKDKDFLDNEENEDILEDIVLTLTLFEDREMIEERLK
TYAHLFDDKVMKQLKRRRYTGWGRLSRKLINGIRDKQSGKTILDFLKSDGFANRNFMQL
IHDDSLTFKEDIQKAQVSGQGDSLHEHIANLAGSPAIKKGILQTVKVVDELVKVMGRHK
PENIVIEMARENQTTQKGQKNSRERMKRIEEGIKELGSQILKEHPVENTQLQNEKLYLY
YLQNGRDMYVDQELDINRLSDYDVDHIVPQSFLKDDSIDNKVLTRSDKNRGKSDNVPSE
EVVKKMKNYWRQLLNAKLITQRKFDNLTKAERGGLSELDKAGFIKRQLVETRQITKHVA
QILDSRMNTKYDENDKLIREVKVITLKSKLVSDFRKDFQFYKVREINNYHHAHDAYLNA
VVGTALIKKYPKLESEFVYGDYKVYDVRKMIAKSEQEIGKATAKYFFYSNIMNFFKTEI
TLANGEIRKRPLIETNGETGEIVWDKGRDFATVRKVLSMPQVNIVKKTEVQTGGFSKES
ILPKRNSDKLIARKKDWDPKKYGGFDSPTVAYSVLVVAKVEKGKSKKLKSVKELLGITI
MERSSFEKNPIDFLEAKGYKEVKKDLIIKLPKYSLFELENGRKRMLASAGELQKGNELA
LPSKYVNFLYLASHYEKLKGSPEDNEQKQLFVEQHKHYLDEIIEQISEFSKRVILADAN
LDKVLSAYNKHRDKPIREQAENIIHLFTLTNLGAPAAFKYFDTTIDRKRYTSTKEVLDA
TLIHQSITGLYETRIDLSQLGGDSGGSKRTADGSEFEPKKKRKV
miniABEmax containing newer ABE8 TadA* variants
(see below), SEQ ID NO: 410
MKRTADGSEFESPKKKRKV[TadA* ABE8e or other ABE8 sequences as
indicated below]SGGSSGGSSGSETPGTSESATPESSGGSSGGSDKKYSIGLAIG
TNSVGWAVITDEYKVPSKKFKVLGNTDRHSIKKNLIGALLFDSGETAEATRLKRTARRR
YTRRKNRICYLQEIFSNEMAKVDDSFFHRLEESFLVEEDKKHERHPIFGNIVDEVAYHE
KYPTIYHLRKKLVDSTDKADLRLIYLALAHMIKFRGHFLIEGDLNPDNSDVDKLFIQLV
QTYNQLFEENPINASGVDAKAILSARLSKSRRLENLIAQLPGEKKNGLFGNLIALSLGL
TPNFKSNFDLAEDAKLQLSKDTYDDDLDNLLAQIGDQYADLFLAAKNLSDAILLSDILR
VNTEITKAPLSASMIKRYDEHHQDLTLLKALVRQQLPEKYKEIFFDQSKNGYAGYIDGG
ASQEEFYKFIKPILEKMDGTEELLVKLNREDLLRKQRTFDNGSIPHQIHLGELHAILRR
QEDFYPFLKDNREKIEKILTFRIPYYVGPLARGNSRFAWMTRKSEETITPWNFEEVVDK
GASAQSFIERMTNFDKNLPNEKVLPKHSLLYEYFTVYNELTKVKYVTEGMRKPAFLSGE
QKKAIVDLLFKTNRKVTVKQLKEDYFKKIECFDSVEISGVEDRFNASLGTYHDLLKIIK
DKDFLDNEENEDILEDIVLTLTLFEDREMIEERLKTYAHLFDDKVMKQLKRRRYTGWGR
LSRKLINGIRDKQSGKTILDFLKSDGFANRNFMQLIHDDSLTFKEDIQKAQVSGQGDSL
HEHIANLAGSPAIKKGILQTVKVVDELVKVMGRHKPENIVIEMARENQTTQKGQKNSRE
RMKRIEEGIKELGSQILKEHPVENTQLQNEKLYLYYLQNGRDMYVDQELDINRLSDYDV
DHIVPQSFLKDDSIDNKVLTRSDKNRGKSDNVPSEEVVKKMKNYWRQLLNAKLITQRKF
DNLTKAERGGLSELDKAGFIKRQLVETRQITKHVAQILDSRMNTKYDENDKLIREVKVI
TLKSKLVSDFRKDFQFYKVREINNYHHAHDAYLNAVVGTALIKKYPKLESEFVYGDYKV
YDVRKMIAKSEQEIGKATAKYFFYSNIMNFFKTEITLANGEIRKRPLIETNGETGEIVW
DKGRDFATVRKVLSMPQVNIVKKTEVQTGGFSKESILPKRNSDKLIARKKDWDPKKYGG
FDSPTVAYSVLVVAKVEKGKSKKLKSVKELLGITIMERSSFEKNPIDFLEAKGYKEVKK
DLIIKLPKYSLFELENGRKRMLASAGELQKGNELALPSKYVNFLYLASHYEKLKGSPED
NEQKQLFVEQHKHYLDEIIEQISEFSKRVILADANLDKVLSAYNKHRDKPIREQAENII
HLFTLTNLGAPAAFKYFDTTIDRKRYTSTKEVLDATLIHQSITGLYETRIDLSQLGGDS
GGSKRTADGSEFEPKKKRKV
TadA* (ABE8e), SEQ ID NO: 411
SEVEFSHEYWMRHALTLAKRARDEREVPVGAVLVLNNRVIGEGWNRAIGLHDPTAHAEI
MALRQGGLVMQNYRLIDATLYVTFEPCVMCAGAMIHSRIGRVVFGVRNSKRGAAGSLMN
VLNYPGMNHRVEITEGILADECAALLCDFYRMPRQVFNAQKKAQSSIN
TadA* (ABE8e_V82G), SEQ ID NO: 412
SEVEFSHEYWMRHALTLAKRARDEREVPVGAVLVLNNRVIGEGWNRAIGLHDPTAHAEI
MALRQGGLVMQNYRLIDATLYGTFEPCVMCAGAMIHSRIGRVVFGVRNSKRGAAGSLMN
VLNYPGMNHRVEITEGILADECAALLCDFYRMPRQVFNAQKKAQSSIN
TadA* (ABE8e_K20A/R21A), SEQ ID NO: 413
SEVEFSHEYWMRHALTLAAAARDEREVPVGAVLVLNNRVIGEGWNRAIGLHDPTAHAEI
MALRQGGLVMQNYRLIDATLYVTFEPCVMCAGAMIHSRIGRVVFGVRNSKRGAAGSLMN
VLNYPGMNHRVEITEGILADECAALLCDFYRMPRQVFNAQKKAQSSIN
TadA* (ABE8.8), SEQ ID NO: 414
SEVEFSHEYWMRHALTLAKRARDEREVPVGAVLVLNNRVIGEGWNRAIGLHDPTAHAEI
MALRQGGLVMQNYRLIDATLYVTFEPCVMCAGAMIHSRIGRVVFGVRNAKTGAAGSLMD
VLHHPGMNHRVEITEGILADECAALLCRFFRMPRRVFNAQKKAQSSTD
TadA* (ABE8.13), SEQ ID NO: 415
SEVEFSHEYWMRHALTLAKRARDEREVPVGAVLVLNNRVIGEGWNRAIGLHDPTAHAEI
MALRQGGLVMQNYRLYDATLYVTFEPCVMCAGAMIHSRIGRVVFGVRNAKTGAAGSLMD
VLHHPGMNHRVEITEGILADECAALLCRFFRMPRRVFNAQKKAQSSTD
TadA* (ABE8.17), SEQ ID NO: 416
SEVEFSHEYWMRHALTLAKRARDEREVPVGAVLVLNNRVIGEGWNRAIGLHDPTAHAEI
MALRQGGLVMQNYRLIDATLYSTFEPCVMCAGAMIHSRIGRVVFGVRNAKTGAAGSLMD
VLHYPGMNHRVEITEGILADECAALLCYFFRMPRRVFNAQKKAQSSTD
TadA* (ABE8.20), SEQ ID NO: 417
SEVEFSHEYWMRHALTLAKRARDEREVPVGAVLVLNNRVIGEGWNRAIGLHDPTAHAEI
MALRQGGLVMQNYRLYDATLYSTFEPCVMCAGAMIHSRIGRVVFGVRNAKTGAAGSLMD
VLHHPGMNHRVEITEGILADECAALLCRFFRMPRRVFNAQKKAQSSTD
SPACE (dual-deaminase base editor), SEQ ID NO: 418
MKRTADGSEFESPKKKRKVSEVEFSHEYWMRHALTLAKRARDEREVPVGAVLVLNNRVI
GEGWNRAIGLHDPTAHAEIMALRQGGLVMQNYRLIDATLYGTFEPCVMCAGAMIHSRIG
RVVFGVRNAKTGAAGSLMDVLHYPGMNHRVEITEGILADECAALLCYFFRMPRQVFNAQ
KKAQSSTDSGGSSGGSSGSETPGTSESATPESSGGSSGGSDKKYSIGLAIGTNSVGWAV
ITDEYKVPSKKFKVLGNTDRHSIKKNLIGALLFDSGETAEATRLKRTARRRYTRRKNRI
CYLQEIFSNEMAKVDDSFFHRLEESFLVEEDKKHERHPIFGNIVDEVAYHEKYPTIYHL
RKKLVDSTDKADLRLIYLALAHMIKFRGHFLIEGDLNPDNSDVDKLFIQLVQTYNQLFE
ENPINASGVDAKAILSARLSKSRRLENLIAQLPGEKKNGLFGNLIALSLGLTPNFKSNF
DLAEDAKLQLSKDTYDDDLDNLLAQIGDQYADLFLAAKNLSDAILLSDILRVNTEITKA
PLSASMIKRYDEHHQDLTLLKALVRQQLPEKYKEIFFDQSKNGYAGYIDGGASQEEFYK
FIKPILEKMDGTEELLVKLNREDLLRKQRTFDNGSIPHQIHLGELHAILRRQEDFYPFL
KDNREKIEKILTFRIPYYVGPLARGNSRFAWMTRKSEETITPWNFEEVVDKGASAQSFI
ERMTNFDKNLPNEKVLPKHSLLYEYFTVYNELTKVKYVTEGMRKPAFLSGEQKKAIVDL
LFKTNRKVTVKQLKEDYFKKIECFDSVEISGVEDRFNASLGTYHDLLKIIKDKDFLDNE
ENEDILEDIVLTLTLFEDREMIEERLKTYAHLFDDKVMKQLKRRRYTGWGRLSRKLING
IRDKQSGKTILDFLKSDGFANRNFMQLIHDDSLTFKEDIQKAQVSGQGDSLHEHIANLA
GSPAIKKGILQTVKVVDELVKVMGRHKPENIVIEMARENQTTQKGQKNSRERMKRIEEG
IKELGSQILKEHPVENTQLQNEKLYLYYLQNGRDMYVDQELDINRLSDYDVDHIVPQSF
LKDDSIDNKVLTRSDKNRGKSDNVPSEEVVKKMKNYWRQLLNAKLITQRKFDNLTKAER
GGLSELDKAGFIKRQLVETRQITKHVAQILDSRMNTKYDENDKLIREVKVITLKSKLVS
DFRKDFQFYKVREINNYHHAHDAYLNAVVGTALIKKYPKLESEFVYGDYKVYDVRKMIA
KSEQEIGKATAKYFFYSNIMNFFKTEITLANGEIRKRPLIETNGETGEIVWDKGRDFAT
VRKVLSMPQVNIVKKTEVQTGGFSKESILPKRNSDKLIARKKDWDPKKYGGFDSPTVAY
SVLVVAKVEKGKSKKLKSVKELLGITIMERSSFEKNPIDFLEAKGYKEVKKDLIIKLPK
YSLFELENGRKRMLASAGELQKGNELALPSKYVNFLYLASHYEKLKGSPEDNEQKQLFV
EQHKHYLDEIIEQISEFSKRVILADANLDKVLSAYNKHRDKPIREQAENIIHLFTLTNL
GAPAAFKYFDTTIDRKRYTSTKEVLDATLIHQSITGLYETRIDLSQLGGDSGGSKRTAD
GSEFEPKKKRKVSGGSSGGSSGSETPGTSESATPESSGGSSGGSTDAEYVRIHEKLDIY
TFKKQFFNNKKSVSHRCYVLFELKRRGERRACFWGYAVNKPQSGTERGIHAEIFSIRKV
EEYLRDNPGQFTINWYSSWSPCADCAEKILEWYNQELRGNGHTLKIWACKLYYEKNARN
QIGLWNLRDNGVGLNVMVSEHYQCCRKIFIQSSHNQLNENRWLEKTLKRAEKWRSELSI
MIQVKILHTTKSPAVSGGSGGSGGSTNLSDIIEKETGKQLVIQESILMLPEEVEEVIGN
KPESDILVHTAYDESTDENVMLLTSDAPEYKPWALVIQDSNGENKIKMLSGGSGGSGGS
TNLSDIIEKETGKQLVIQESILMLPEEVEEVIGNKPESDILVHTAYDESTDENVMLLTS
DAPEYKPWALVIQDSNGENKIKMLGSGATNFSLLKQAGDVEENPGPMVSKGEELFTGVV
PILVELDGDVNGHKFSVSGEGEGDATYGKLTLKFICTTGKLPVPWPTLVTTLTYGVQCF
SRYPDHMKQHDFFKSAMPEGYVQERTIFFKDDGNYKTRAEVKFEGDTLVNRIELKGIDF
KEDGNILGHKLEYNYNSHNVYIMADKQKNGIKVNFKIRHNIEDGSVQLADHYQQNTPIG
DGPVLLPDNHYLSTQSALSKDPNEKRDHMVLLEFVTAAGITLGMDELYKSGGSPKKKRK
SPACE-ΔUGI (SPACE without UGI fusion), SEQ
ID NO: 419
MKRTADGSEFESPKKKRKVSEVEFSHEYWMRHALTLAKRARDEREVPVGAVLVLNNRVI
GEGWNRAIGLHDPTAHAEIMALRQGGLVMQNYRLIDATLYGTFEPCVMCAGAMIHSRIG
RVVFGVRNAKTGAAGSLMDVLHYPGMNHRVEITEGILADECAALLCYFFRMPRQVFNAQ
KKAQSSTDSGGSSGGSSGSETPGTSESATPESSGGSSGGSDKKYSIGLAIGTNSVGWAV
ITDEYKVPSKKFKVLGNTDRHSIKKNLIGALLFDSGETAEATRLKRTARRRYTRRKNRI
CYLQEIFSNEMAKVDDSFFHRLEESFLVEEDKKHERHPIFGNIVDEVAYHEKYPTIYHL
RKKLVDSTDKADLRLIYLALAHMIKFRGHFLIEGDLNPDNSDVDKLFIQLVQTYNQLFE
ENPINASGVDAKAILSARLSKSRRLENLIAQLPGEKKNGLFGNLIALSLGLTPNFKSNF
DLAEDAKLQLSKDTYDDDLDNLLAQIGDQYADLFLAAKNLSDAILLSDILRVNTEITKA
PLSASMIKRYDEHHQDLTLLKALVRQQLPEKYKEIFFDQSKNGYAGYIDGGASQEEFYK
FIKPILEKMDGTEELLVKLNREDLLRKQRTFDNGSIPHQIHLGELHAILRRQEDFYPFL
KDNREKIEKILTFRIPYYVGPLARGNSRFAWMTRKSEETITPWNFEEVVDKGASAQSFI
ERMTNFDKNLPNEKVLPKHSLLYEYFTVYNELTKVKYVTEGMRKPAFLSGEQKKAIVDL
LFKTNRKVTVKQLKEDYFKKIECFDSVEISGVEDRFNASLGTYHDLLKIIKDKDFLDNE
ENEDILEDIVLTLTLFEDREMIEERLKTYAHLFDDKVMKQLKRRRYTGWGRLSRKLING
IRDKQSGKTILDFLKSDGFANRNFMQLIHDDSLTFKEDIQKAQVSGQGDSLHEHIANLA
GSPAIKKGILQTVKVVDELVKVMGRHKPENIVIEMARENQTTQKGQKNSRERMKRIEEG
IKELGSQILKEHPVENTQLQNEKLYLYYLQNGRDMYVDQELDINRLSDYDVDHIVPQSF
LKDDSIDNKVLTRSDKNRGKSDNVPSEEVVKKMKNYWRQLLNAKLITQRKFDNLTKAER
GGLSELDKAGFIKRQLVETRQITKHVAQILDSRMNTKYDENDKLIREVKVITLKSKLVS
DFRKDFQFYKVREINNYHHAHDAYLNAVVGTALIKKYPKLESEFVYGDYKVYDVRKMIA
KSEQEIGKATAKYFFYSNIMNFFKTEITLANGEIRKRPLIETNGETGEIVWDKGRDFAT
VRKVLSMPQVNIVKKTEVQTGGFSKESILPKRNSDKLIARKKDWDPKKYGGFDSPTVAY
SVLVVAKVEKGKSKKLKSVKELLGITIMERSSFEKNPIDFLEAKGYKEVKKDLIIKLPK
YSLFELENGRKRMLASAGELQKGNELALPSKYVNFLYLASHYEKLKGSPEDNEQKQLFV
EQHKHYLDEIIEQISEFSKRVILADANLDKVLSAYNKHRDKPIREQAENIIHLFTLTNL
GAPAAFKYFDTTIDRKRYTSTKEVLDATLIHQSITGLYETRIDLSQLGGDSGGSKRTAD
GSEFEPKKKRKVSGGSSGGSSGSETPGTSESATPESSGGSSGGSTDAEYVRIHEKLDIY
TFKKQFFNNKKSVSHRCYVLFELKRRGERRACFWGYAVNKPQSGTERGIHAEIFSIRKV
EEYLRDNPGQFTINWYSSWSPCADCAEKILEWYNQELRGNGHTLKIWACKLYYEKNARN
QIGLWNLRDNGVGLNVMVSEHYQCCRKIFIQSSHNQLNENRWLEKTLKRAEKWRSELSI
MIQVKILHTTKSPAVGSGATNFSLLKQAGDVEENPGPMVSKGEELFTGVVPILVELDGD
VNGHKFSVSGEGEGDATYGKLTLKFICTTGKLPVPWPTLVTTLTYGVQCFSRYPDHMKQ
HDFFKSAMPEGYVQERTIFFKDDGNYKTRAEVKFEGDTLVNRIELKGIDFKEDGNILGH
KLEYNYNSHNVYIMADKQKNGIKVNFKIRHNIEDGSVQLADHYQQNTPIGDGPVLLPDN
HYLSTQSALSKDPNEKRDHMVLLEFVTAAGITLGMDELYKSGGSPKKKRKV
SPACE-NG, SEQ ID NO: 420
MKRTADGSEFESPKKKRKVSEVEFSHEYWMRHALTLAKRARDEREVPVGAVLVLNNRVI
GEGWNRAIGLHDPTAHAEIMALRQGGLVMQNYRLIDATLYGTFEPCVMCAGAMIHSRIG
RVVFGVRNAKTGAAGSLMDVLHYPGMNHRVEITEGILADECAALLCYFFRMPRQVFNAQ
KKAQSSTDSGGSSGGSSGSETPGTSESATPESSGGSSGGSDKKYSIGLAIGTNSVGWAV
ITDEYKVPSKKFKVLGNTDRHSIKKNLIGALLFDSGETAEATRLKRTARRRYTRRKNRI
CYLQEIFSNEMAKVDDSFFHRLEESFLVEEDKKHERHPIFGNIVDEVAYHEKYPTIYHL
RKKLVDSTDKADLRLIYLALAHMIKFRGHFLIEGDLNPDNSDVDKLFIQLVQTYNQLFE
ENPINASGVDAKAILSARLSKSRRLENLIAQLPGEKKNGLFGNLIALSLGLTPNFKSNF
DLAEDAKLQLSKDTYDDDLDNLLAQIGDQYADLFLAAKNLSDAILLSDILRVNTEITKA
PLSASMIKRYDEHHQDLTLLKALVRQQLPEKYKEIFFDQSKNGYAGYIDGGASQEEFYK
FIKPILEKMDGTEELLVKLNREDLLRKQRTFDNGSIPHQIHLGELHAILRRQEDFYPFL
KDNREKIEKILTFRIPYYVGPLARGNSRFAWMTRKSEETITPWNFEEVVDKGASAQSFI
ERMTNFDKNLPNEKVLPKHSLLYEYFTVYNELTKVKYVTEGMRKPAFLSGEQKKAIVDL
LFKTNRKVTVKQLKEDYFKKIECFDSVEISGVEDRFNASLGTYHDLLKIIKDKDFLDNE
ENEDILEDIVLTLTLFEDREMIEERLKTYAHLFDDKVMKQLKRRRYTGWGRLSRKLING
IRDKQSGKTILDFLKSDGFANRNFMQLIHDDSLTFKEDIQKAQVSGQGDSLHEHIANLA
GSPAIKKGILQTVKVVDELVKVMGRHKPENIVIEMARENQTTQKGQKNSRERMKRIEEG
IKELGSQILKEHPVENTQLQNEKLYLYYLQNGRDMYVDQELDINRLSDYDVDHIVPQSF
LKDDSIDNKVLTRSDKNRGKSDNVPSEEVVKKMKNYWRQLLNAKLITQRKFDNLTKAER
GGLSELDKAGFIKRQLVETRQITKHVAQILDSRMNTKYDENDKLIREVKVITLKSKLVS
DFRKDFQFYKVREINNYHHAHDAYLNAVVGTALIKKYPKLESEFVYGDYKVYDVRKMIA
KSEQEIGKATAKYFFYSNIMNFFKTEITLANGEIRKRPLIETNGETGEIVWDKGRDFAT
VRKVLSMPQVNIVKKTEVQTGGFSKESIRPKRNSDKLIARKKDWDPKKYGGFVSPTVAY
SVLVVAKVEKGKSKKLKSVKELLGITIMERSSFEKNPIDFLEAKGYKEVKKDLIIKLPK
YSLFELENGRKRMLASARFLQKGNELALPSKYVNFLYLASHYEKLKGSPEDNEQKQLFV
EQHKHYLDEIIEQISEFSKRVILADANLDKVLSAYNKHRDKPIREQAENIIHLFTLTNL
GAPRAFKYFDTTIDRKVYRSTKEVLDATLIHQSITGLYETRIDLSQLGGDSGGSKRTAD
GSEFEPKKKRKVSGGSSGGSSGSETPGTSESATPESSGGSSGGSTDAEYVRIHEKLDIY
TFKKQFFNNKKSVSHRCYVLFELKRRGERRACFWGYAVNKPQSGTERGIHAEIFSIRKV
EEYLRDNPGQFTINWYSSWSPCADCAEKILEWYNQELRGNGHTLKIWACKLYYEKNARN
QIGLWNLRDNGVGLNVMVSEHYQCCRKIFIQSSHNQLNENRWLEKTLKRAEKWRSELSI
MIQVKILHTTKSPAVSGGSGGSGGSTNLSDIIEKETGKQLVIQESILMLPEEVEEVIGN
KPESDILVHTAYDESTDENVMLLTSDAPEYKPWALVIQDSNGENKIKMLSGGSGGSGGS
TNLSDIIEKETGKQLVIQESILMLPEEVEEVIGNKPESDILVHTAYDESTDENVMLLTS
DAPEYKPWALVIQDSNGENKIKMLGSGATNFSLLKQAGDVEENPGPMVSKGEELFTGVV
PILVELDGDVNGHKFsVSGEGEGDATYGKLTLKFICTTGKLPVPWPTLVTTLTYGVQCF
SRYPDHMKQHDFFKSAMPEGYVQERTIFFKDDGNYKTRAEVKFEGDTLVNRIELKGIDF
KEDGNILGHKLEYNYNSHNVYIMADKQKNGIKVNFKIRHNIEDGSVQLADHYQQNTPIG
DGPVLLPDNHYLSTQSALSKDPNEKRDHMVLLEFVTAAGITLGMDELYKSGGSPKKKRK
V
SPACE-VRQR, SEQ ID NO: 421
MKRTADGSEFESPKKKRKVSEVEFSHEYWMRHALTLAKRARDEREVPVGAVLVLNNRVI
GEGWNRAIGLHDPTAHAEIMALRQGGLVMQNYRLIDATLYGTFEPCVMCAGAMIHSRIG
RVVFGVRNAKTGAAGSLMDVLHYPGMNHRVEITEGILADECAALLCYFFRMPRQVFNAQ
KKAQSSTDSGGSSGGSSGSETPGTSESATPESSGGSSGGSDKKYSIGLAIGTNSVGWAV
ITDEYKVPSKKFKVLGNTDRHSIKKNLIGALLFDSGETAEATRLKRTARRRYTRRKNRI
CYLQEIFSNEMAKVDDSFFHRLEESFLVEEDKKHERHPIFGNIVDEVAYHEKYPTIYHL
RKKLVDSTDKADLRLIYLALAHMIKFRGHFLIEGDLNPDNSDVDKLFIQLVQTYNQLFE
ENPINASGVDAKAILSARLSKSRRLENLIAQLPGEKKNGLFGNLIALSLGLTPNFKSNF
DLAEDAKLQLSKDTYDDDLDNLLAQIGDQYADLFLAAKNLSDAILLSDILRVNTEITKA
PLSASMIKRYDEHHQDLTLLKALVRQQLPEKYKEIFFDQSKNGYAGYIDGGASQEEFYK
FIKPILEKMDGTEELLVKLNREDLLRKQRTFDNGSIPHQIHLGELHAILRRQEDFYPFL
KDNREKIEKILTFRIPYYVGPLARGNSRFAWMTRKSEETITPWNFEEVVDKGASAQSFI
ERMTNFDKNLPNEKVLPKHSLLYEYFTVYNELTKVKYVTEGMRKPAFLSGEQKKAIVDL
LFKTNRKVTVKQLKEDYFKKIECFDSVEISGVEDRFNASLGTYHDLLKIIKDKDFLDNE
ENEDILEDIVLTLTLFEDREMIEERLKTYAHLFDDKVMKQLKRRRYTGWGRLSRKLING
IRDKQSGKTILDFLKSDGFANRNFMQLIHDDSLTFKEDIQKAQVSGQGDSLHEHIANLA
GSPAIKKGILQTVKVVDELVKVMGRHKPENIVIEMARENQTTQKGQKNSRERMKRIEEG
IKELGSQILKEHPVENTQLQNEKLYLYYLQNGRDMYVDQELDINRLSDYDVDHIVPQSF
LKDDSIDNKVLTRSDKNRGKSDNVPSEEVVKKMKNYWRQLLNAKLITQRKFDNLTKAER
GGLSELDKAGFIKRQLVETRQITKHVAQILDSRMNTKYDENDKLIREVKVITLKSKLVS
DFRKDFQFYKVREINNYHHAHDAYLNAVVGTALIKKYPKLESEFVYGDYKVYDVRKMIA
KSEQEIGKATAKYFFYSNIMNFFKTEITLANGEIRKRPLIETNGETGEIVWDKGRDFAT
VRKVLSMPQVNIVKKTEVQTGGFSKESILPKRNSDKLIARKKDWDPKKYGGFVSPTVAY
SVLVVAKVEKGKSKKLKSVKELLGITIMERSSFEKNPIDFLEAKGYKEVKKDLIIKLPK
YSLFELENGRKRMLASARELQKGNELALPSKYVNFLYLASHYEKLKGSPEDNEQKQLFV
EQHKHYLDEIIEQISEFSKRVILADANLDKVLSAYNKHRDKPIREQAENIIHLFTLTNL
GAPAAFKYFDTTIDRKQYRSTKEVLDATLIHQSITGLYETRIDLSQLGGDSGGSKRTAD
GSEFEPKKKRKVSGGSSGGSSGSETPGTSESATPESSGGSSGGSTDAEYVRIHEKLDIY
TFKKQFFNNKKSVSHRCYVLFELKRRGERRACFWGYAVNKPQSGTERGIHAEIFSIRKV
EEYLRDNPGQFTINWYSSWSPCADCAEKILEWYNQELRGNGHTLKIWACKLYYEKNARN
QIGLWNLRDNGVGLNVMVSEHYQCCRKIFIQSSHNQLNENRWLEKTLKRAEKWRSELSI
MIQVKILHTTKSPAVSGGSGGSGGSTNLSDIIEKETGKQLVIQESILMLPEEVEEVIGN
KPESDILVHTAYDESTDENVMLLTSDAPEYKPWALVIQDSNGENKIKMLSGGSGGSGGS
TNLSDIIEKETGKQLVIQESILMLPEEVEEVIGNKPESDILVHTAYDESTDENVMLLTS
DAPEYKPWALVIQDSNGENKIKMLGSGATNFSLLKQAGDVEENPGPMVSKGEELFTGVV
PILVELDGDVNGHKFsVSGEGEGDATYGKLTLKFICTTGKLPVPWPTLVTTLTYGVQCF
SRYPDHMKQHDFFKSAMPEGYVQERTIFFKDDGNYKTRAEVKFEGDTLVNRIELKGIDF
KEDGNILGHKLEYNYNSHNVYIMADKQKNGIKVNFKIRHNIEDGSVQLADHYQQNTPIG
DGPVLLPDNHYLSTQSALSKDPNEKRDHMVLLEFVTAAGITLGMDELYKSGGSPKKKRK
V
SPACE-NAA, SEQ ID NO: 422
MKRTADGSEFESPKKKRKVSEVEFSHEYWMRHALTLAKRARDEREVPVGAVLVLNNRVI
GEGWNRAIGLHDPTAHAEIMALRQGGLVMQNYRLIDATLYGTFEPCVMCAGAMIHSRIG
RVVFGVRNAKTGAAGSLMDVLHYPGMNHRVEITEGILADECAALLCYFFRMPRQVFNAQ
KKAQSSTDSGGSSGGSSGSETPGTSESATPESSGGSSGGSDKKYSIGLAIGTNSVGWAV
ITDEYKVPSKKFKVLGNTDRHSIKKNLIGALLFDSGETAEATRLKRTARRRYTRRKNRI
CYLQEIFSNEMAKVDDSFFHRLEESFLVEEDKKHERHPIFGNIVDEVAYHEKYPTIYHL
RKKLVDSTDKADLRLIYLALAHMIKFRGHFLIEGDLNPDNSDVDKLFIQLVQTYNQLFE
ENPINASGVDAKAILSARLSKSRRLENLIAQLPGEKKNGLFGNLIALSLGLTPNFKSNF
DLAEDAKLQLSKDTYDDDLDNLLAQIGDQYADLFLAAKNLSDAILLSDILRVNTEITKA
PLSASMIKRYDEHHQDLTLLKALVRQQLPEKYKEIFFDQSKNGYAGYIDGGASQEEFYK
FIKPILEKMDGTEELLVKLNREDLLRKQRTFDNGSIPHQIHLGELHAILRRQEDFYPFL
KDNREKIEKILTFRIPYYVGPLARGNSRFAWMTRKSEETITPWNFEEVVDKGASAQSFI
ERMTNFDKNLPNEKVLPKHSLLYEYFTVYNELTKVKYVTEGMRKPAFLSGEQKKAIVDL
LFKTNRKVTVKQLKEDYFKKIECFDSVEISGVEDRFNASLGTYHDLLKIIKDKDFLDNE
ENEDILEDIVLTLTLFEDREMIEERLKTYAHLFDDKVMKQLKRRRYTGWGRLSRKLING
IRDKQSGKTILDFLKSDGFANRNFMQLIHDDSLTFKEDIQKAQVSGQGDSLHEHIANLA
GSPAIKKGILQTVKVVDELVKVMGRHKPENIVIEMARENQTTQKGQKNSRERMKRIEEG
IKELGSQILKEHPVENTQLQNEKLYLYYLQNGRDMYVDQELDINRLSDYDVDHIVPQSF
LKDDSIDNKVLTRSDKNRGKSDNVPSEEVVKKMKNYWRQLLNAKLITQRKFDNLTKAER
GGLSELDKAGFIKRQLVETRQITKHVAQILDSRMNTKYDENDKLIREVKVITLKSKLVS
DFRKDFQFYKVREINNYHHAHDAYLNAVVGTALIKKYPKLESEFVYGDYKVYDVRKMIA
KSEQEIGKATAKYFFYSNIMNFFKTEITLANGEIRKRPLIETNGETGEIVWDKGRDFAT
VRKVLSMPQVNIVKKTEIQTVGQNGGLFDDNPKSPLEVTPSKLVPLKKELNPKKYGGYQ
KPTTAYPVLLITDTKQLIPISVMNKKQFEQNPVKFLRDRGYQQVGKNDFIKLPKYTLVD
IGDGIKRLWASSKEIHKGNQLVVSKKSQILLYHAHHLDSDLSNDYLQNHNQQFDVLFNE
IISFSKKCKLGKEHIQKIENVYSNKKNSASIEELAESFIKLLGFTQLGATSPFNFLGVK
LNQKQYKGKKDYILPCTEGTLIRQSITGLYETRVDLSKIGEDSGGSKRTADGSEFEPKK
KRKVSGGSSGGSSGSETPGTSESATPESSGGSSGGSTDAEYVRIHEKLDIYTFKKQFFN
NKKSVSHRCYVLFELKRRGERRACFWGYAVNKPQSGTERGIHAEIFSIRKVEEYLRDNP
GQFTINWYSSWSPCADCAEKILEWYNQELRGNGHTLKIWACKLYYEKNARNQIGLWNLR
DNGVGLNVMVSEHYQCCRKIFIQSSHNQLNENRWLEKTLKRAEKWRSELSIMIQVKILH
TTKSPAVSGGSGGSGGSTNLSDIIEKETGKQLVIQESILMLPEEVEEVIGNKPESDILV
HTAYDESTDENVMLLTSDAPEYKPWALVIQDSNGENKIKMLSGGSGGSGGSTNLSDIIE
KETGKQLVIQESILMLPEEVEEVIGNKPESDILVHTAYDESTDENVMLLTSDAPEYKPW
ALVIQDSNGENKIKMLGSGATNFSLLKQAGDVEENPGPMVSKGEELFTGVVPILVELDG
DVNGHKFSVSGEGEGDATYGKLTLKFICTTGKLPVPWPTLVTTLTYGVQCFSRYPDHMK
QHDFFKSAMPEGYVQERTIFFKDDGNYKTRAEVKFEGDTLVNRIELKGIDFKEDGNILG
HKLEYNYNSHNVYIMADKQKNGIKVNFKIRHNIEDGSVQLADHYQQNTPIGDGPVLLPD
NHYLSTQSALSKDPNEKRDHMVLLEFVTAAGITLGMDELYKSGGSPKKKRKV
BE4max(R33A)-ΔUGI-eUNG, SEQ ID NO: 423
MKRTADGSEFESPKKKRKVSSETGPVAVDPTLRRRIEPHEFEVFFDPRELAKETCLLYE
INWGGRHSIWRHTSQNTNKHVEVNFIEKFTTERYFCPNTRCSITWFLSWSPCGECSRAI
TEFLSRYPHVTLFIYIARLYHHADPRNRQGLRDLISSGVTIQIMTEQESGYCWRNFVNY
SPSNEAHWPRYPHLWVRLYVLELYCIILGLPPCLNILRRKQPQLTFFTIALQSCHYQRL
PPHILWATGLKSGGSSGGSSGSETPGTSESATPESSGGSSGGSDKKYSIGLAIGTNSVG
WAVITDEYKVPSKKFKVLGNTDRHSIKKNLIGALLFDSGETAEATRLKRTARRRYTRRK
NRICYLQEIFSNEMAKVDDSFFHRLEESFLVEEDKKHERHPIFGNIVDEVAYHEKYPTI
YHLRKKLVDSTDKADLRLIYLALAHMIKFRGHFLIEGDLNPDNSDVDKLFIQLVQTYNQ
LFEENPINASGVDAKAILSARLSKSRRLENLIAQLPGEKKNGLFGNLIALSLGLTPNFK
SNFDLAEDAKLQLSKDTYDDDLDNLLAQIGDQYADLFLAAKNLSDAILLSDILRVNTEI
TKAPLSASMIKRYDEHHQDLTLLKALVRQQLPEKYKEIFFDQSKNGYAGYIDGGASQEE
FYKFIKPILEKMDGTEELLVKLNREDLLRKQRTFDNGSIPHQIHLGELHAILRRQEDFY
PFLKDNREKIEKILTFRIPYYVGPLARGNSRFAWMTRKSEETITPWNFEEVVDKGASAQ
SFIERMTNFDKNLPNEKVLPKHSLLYEYFTVYNELTKVKYVTEGMRKPAFLSGEQKKAI
VDLLFKTNRKVTVKQLKEDYFKKIECFDSVEISGVEDRFNASLGTYHDLLKIIKDKDFL
DNEENEDILEDIVLTLTLFEDREMIEERLKTYAHLFDDKVMKQLKRRRYTGWGRLSRKL
INGIRDKQSGKTILDFLKSDGFANRNFMQLIHDDSLTFKEDIQKAQVSGQGDSLHEHIA
NLAGSPAIKKGILQTVKVVDELVKVMGRHKPENIVIEMARENQTTQKGQKNSRERMKRI
EEGIKELGSQILKEHPVENTQLQNEKLYLYYLQNGRDMYVDQELDINRLSDYDVDHIVP
QSFLKDDSIDNKVLTRSDKNRGKSDNVPSEEVVKKMKNYWRQLLNAKLITQRKFDNLTK
AERGGLSELDKAGFIKRQLVETRQITKHVAQILDSRMNTKYDENDKLIREVKVITLKSK
LVSDFRKDFQFYKVREINNYHHAHDAYLNAVVGTALIKKYPKLESEFVYGDYKVYDVRK
MIAKSEQEIGKATAKYFFYSNIMNFFKTEITLANGEIRKRPLIETNGETGEIVWDKGRD
FATVRKVLSMPQVNIVKKTEVQTGGFSKESILPKRNSDKLIARKKDWDPKKYGGFDSPT
VAYSVLVVAKVEKGKSKKLKSVKELLGITIMERSSFEKNPIDFLEAKGYKEVKKDLIIK
LPKYSLFELENGRKRMLASAGELQKGNELALPSKYVNFLYLASHYEKLKGSPEDNEQKQ
LFVEQHKHYLDEIIEQISEFSKRVILADANLDKVLSAYNKHRDKPIREQAENIIHLFTL
TNLGAPAAFKYFDTTIDRKRYTSTKEVLDATLIHQSITGLYETRIDLSQLGGDSGGSGG
SGGSANELTWHDVLAEEKQQPYFLNTLQTVASERQSGVTIYPPQKDVFNAFRFTELGDV
KVVILGQDPYHGPGQAHGLAFSVRPGIAIPPSLLNMYKELENTIPGFTRPNHGYLESWA
RQGVLLLNTVLTVRAGQAHSHASLGWETFTDKVISLINQHREGVVFLLWGSHAQKKGAI
IDKQRHHVLKAPHPSPLSAHRGFFGCNHFVLANQWLEQRGETPIDWMPVLPAESESGGS
KRTADGSEFEPKKKRKVGGGGSGATNFSLLKQAGDVEENPGPMVSKGEELFTGVVPILV
ELDGDVNGHKFSVSGEGEGDATYGKLTLKFICTTGKLPVPWPTLVTTLTYGVQCFSRYP
DHMKQHDFFKSAMPEGYVQERTIFFKDDGNYKTRAEVKFEGDTLVNRIELKGIDFKEDG
NILGHKLEYNYNSHNVYIMADKQKNGIKVNFKIRHNIEDGSVQLADHYQQNTPIGDGPV
LLPDNHYLSTQSALSKDPNEKRDHMVLLEFVTAAGITLGMDELYK
eUNG-BE4max(R33A)-ΔUGI = CGBE1, SEQ ID NO: 424
MKRTADGSEFESPKKKRKVANELTWHDVLAEEKQQPYFLNTLQTVASERQSGVTIYPPQ
KDVFNAFRFTELGDVKVVILGQDPYHGPGQAHGLAFSVRPGIAIPPSLLNMYKELENTI
PGFTRPNHGYLESWARQGVLLLNTVLTVRAGQAHSHASLGWETFTDKVISLINQHREGV
VFLLWGSHAQKKGAIIDKQRHHVLKAPHPSPLSAHRGFFGCNHFVLANQWLEQRGETPI
DWMPVLPAESESGGSGGSGGSSSETGPVAVDPTLRRRIEPHEFEVFFDPRELAKETCLL
YEINWGGRHSIWRHTSQNTNKHVEVNFIEKFTTERYFCPNTRCSITWFLSWSPCGECSR
AITEFLSRYPHVTLFIYIARLYHHADPRNRQGLRDLISSGVTIQIMTEQESGYCWRNFV
NYSPSNEAHWPRYPHLWVRLYVLELYCIILGLPPCLNILRRKQPQLTFFTIALQSCHYQ
RLPPHILWATGLKSGGSSGGSSGSETPGTSESATPESSGGSSGGSDKKYSIGLAIGTNS
VGWAVITDEYKVPSKKFKVLGNTDRHSIKKNLIGALLFDSGETAEATRLKRTARRRYTR
RKNRICYLQEIFSNEMAKVDDSFFHRLEESFLVEEDKKHERHPIFGNIVDEVAYHEKYP
TIYHLRKKLVDSTDKADLRLIYLALAHMIKFRGHFLIEGDLNPDNSDVDKLFIQLVQTY
NQLFEENPINASGVDAKAILSARLSKSRRLENLIAQLPGEKKNGLFGNLIALSLGLTPN
FKSNFDLAEDAKLQLSKDTYDDDLDNLLAQIGDQYADLFLAAKNLSDAILLSDILRVNT
EITKAPLSASMIKRYDEHHQDLTLLKALVRQQLPEKYKEIFFDQSKNGYAGYIDGGASQ
EEFYKFIKPILEKMDGTEELLVKLNREDLLRKQRTFDNGSIPHQIHLGELHAILRRQED
FYPFLKDNREKIEKILTFRIPYYVGPLARGNSRFAWMTRKSEETITPWNFEEVVDKGAS
AQSFIERMTNFDKNLPNEKVLPKHSLLYEYFTVYNELTKVKYVTEGMRKPAFLSGEQKK
AIVDLLFKTNRKVTVKQLKEDYFKKIECFDSVEISGVEDRFNASLGTYHDLLKIIKDKD
FLDNEENEDILEDIVLTLTLFEDREMIEERLKTYAHLFDDKVMKQLKRRRYTGWGRLSR
KLINGIRDKQSGKTILDFLKSDGFANRNFMQLIHDDSLTFKEDIQKAQVSGQGDSLHEH
IANLAGSPAIKKGILQTVKVVDELVKVMGRHKPENIVIEMARENQTTQKGQKNSRERMK
RIEEGIKELGSQILKEHPVENTQLQNEKLYLYYLQNGRDMYVDQELDINRLSDYDVDHI
VPQSFLKDDSIDNKVLTRSDKNRGKSDNVPSEEVVKKMKNYWRQLLNAKLITQRKFDNL
TKAERGGLSELDKAGFIKRQLVETRQITKHVAQILDSRMNTKYDENDKLIREVKVITLK
SKLVSDFRKDFQFYKVREINNYHHAHDAYLNAVVGTALIKKYPKLESEFVYGDYKVYDV
RKMIAKSEQEIGKATAKYFFYSNIMNFFKTEITLANGEIRKRPLIETNGETGEIVWDKG
RDFATVRKVLSMPQVNIVKKTEVQTGGFSKESILPKRNSDKLIARKKDWDPKKYGGFDS
PTVAYSVLVVAKVEKGKSKKLKSVKELLGITIMERSSFEKNPIDFLEAKGYKEVKKDLI
IKLPKYSLFELENGRKRMLASAGELQKGNELALPSKYVNFLYLASHYEKLKGSPEDNEQ
KQLFVEQHKHYLDEIIEQISEFSKRVILADANLDKVLSAYNKHRDKPIREQAENIIHLF
TLTNLGAPAAFKYFDTTIDRKRYTSTKEVLDATLIHQSITGLYETRIDLSQLGGDSGGS
KRTADGSEFEPKKKRKVGGGGSGATNFSLLKQAGDVEENPGPMVSKGEELFTGVVPILV
ELDGDVNGHKFSVSGEGEGDATYGKLTLKFICTTGKLPVPWPTLVTTLTYGVQCFSRYP
DHMKQHDFFKSAMPEGYVQERTIFFKDDGNYKTRAEVKFEGDTLVNRIELKGIDFKEDG
NILGHKLEYNYNSHNVYIMADKQKNGIKVNFKIRHNIEDGSVQLADHYQQNTPIGDGPV
LLPDNHYLSTQSALSKDPNEKRDHMVLLEFVTAAGITLGMDELYK
BE4max-ΔUGI, SEQ ID NO: 425
MKRTADGSEFESPKKKRKVSSETGPVAVDPTLRRRIEPHEFEVFFDPRELRKETCLLYE
INWGGRHSIWRHTSQNTNKHVEVNFIEKFTTERYFCPNTRCSITWFLSWSPCGECSRAI
TEFLSRYPHVTLFIYIARLYHHADPRNRQGLRDLISSGVTIQIMTEQESGYCWRNFVNY
SPSNEAHWPRYPHLWVRLYVLELYCIILGLPPCLNILRRKQPQLTFFTIALQSCHYQRL
PPHILWATGLKSGGSSGGSSGSETPGTSESATPESSGGSSGGSDKKYSIGLAIGTNSVG
WAVITDEYKVPSKKFKVLGNTDRHSIKKNLIGALLFDSGETAEATRLKRTARRRYTRRK
NRICYLQEIFSNEMAKVDDSFFHRLEESFLVEEDKKHERHPIFGNIVDEVAYHEKYPTI
YHLRKKLVDSTDKADLRLIYLALAHMIKFRGHFLIEGDLNPDNSDVDKLFIQLVQTYNQ
LFEENPINASGVDAKAILSARLSKSRRLENLIAQLPGEKKNGLFGNLIALSLGLTPNFK
SNFDLAEDAKLQLSKDTYDDDLDNLLAQIGDQYADLFLAAKNLSDAILLSDILRVNTEI
TKAPLSASMIKRYDEHHQDLTLLKALVRQQLPEKYKEIFFDQSKNGYAGYIDGGASQEE
FYKFIKPILEKMDGTEELLVKLNREDLLRKQRTFDNGSIPHQIHLGELHAILRRQEDFY
PFLKDNREKIEKILTFRIPYYVGPLARGNSRFAWMTRKSEETITPWNFEEVVDKGASAQ
SFIERMTNFDKNLPNEKVLPKHSLLYEYFTVYNELTKVKYVTEGMRKPAFLSGEQKKAI
VDLLFKTNRKVTVKQLKEDYFKKIECFDSVEISGVEDRFNASLGTYHDLLKIIKDKDFL
DNEENEDILEDIVLTLTLFEDREMIEERLKTYAHLFDDKVMKQLKRRRYTGWGRLSRKL
INGIRDKQSGKTILDFLKSDGFANRNFMQLIHDDSLTFKEDIQKAQVSGQGDSLHEHIA
NLAGSPAIKKGILQTVKVVDELVKVMGRHKPENIVIEMARENQTTQKGQKNSRERMKRI
EEGIKELGSQILKEHPVENTQLQNEKLYLYYLQNGRDMYVDQELDINRLSDYDVDHIVP
QSFLKDDSIDNKVLTRSDKNRGKSDNVPSEEVVKKMKNYWRQLLNAKLITQRKFDNLTK
AERGGLSELDKAGFIKRQLVETRQITKHVAQILDSRMNTKYDENDKLIREVKVITLKSK
LVSDFRKDFQFYKVREINNYHHAHDAYLNAVVGTALIKKYPKLESEFVYGDYKVYDVRK
MIAKSEQEIGKATAKYFFYSNIMNFFKTEITLANGEIRKRPLIETNGETGEIVWDKGRD
FATVRKVLSMPQVNIVKKTEVQTGGFSKESILPKRNSDKLIARKKDWDPKKYGGFDSPT
VAYSVLVVAKVEKGKSKKLKSVKELLGITIMERSSFEKNPIDFLEAKGYKEVKKDLIIK
LPKYSLFELENGRKRMLASAGELQKGNELALPSKYVNFLYLASHYEKLKGSPEDNEQKQ
LFVEQHKHYLDEIIEQISEFSKRVILADANLDKVLSAYNKHRDKPIREQAENIIHLFTL
TNLGAPAAFKYFDTTIDRKRYTSTKEVLDATLIHQSITGLYETRIDLSQLGGDSGGSKR
TADGSEFEPKKKRKVGGGGSGATNFSLLKQAGDVEENPGPMVSKGEELFTGVVPILVEL
DGDVNGHKFSVSGEGEGDATYGKLTLKFICTTGKLPVPWPTLVTTLTYGVQCFSRYPDH
MKQHDFFKSAMPEGYVQERTIFFKDDGNYKTRAEVKFEGDTLVNRIELKGIDFKEDGNI
LGHKLEYNYNSHNVYIMADKQKNGIKVNFKIRHNIEDGSVQLADHYQQNTPIGDGPVLL
PDNHYLSTQSALSKDPNEKRDHMVLLEFVTAAGITLGMDELYK
eA3A-BE3-ΔUGI-eUNG, SEQ ID NO: 426
MEASPASGPRHLMDPHIFTSNFNNGIGRHKTYLCYEVERLDNGTSVKMDQHRGFLHGQA
KNLLCGFYGRHAELRFLDLVPSLQLDPAQIYRVTWFISWSPCFSWGCAGEVRAFLQENT
HVRLRIFAARIYDYDPLYKEALQMLRDAGAQVSIMTYDEFKHCWDTFVDHQGCPFQPWD
GLDEHSQALSGRLRAILQNQGNSGSETPGTSESATPESDKKYSIGLAIGTNSVGWAVIT
DEYKVPSKKFKVLGNTDRHSIKKNLIGALLFDSGETAEATRLKRTARRRYTRRKNRICY
LQEIFSNEMAKVDDSFFHRLEESFLVEEDKKHERHPIFGNIVDEVAYHEKYPTIYHLRK
KLVDSTDKADLRLIYLALAHMIKFRGHFLIEGDLNPDNSDVDKLFIQLVQTYNQLFEEN
PINASGVDAKAILSARLSKSRRLENLIAQLPGEKKNGLFGNLIALSLGLTPNFKSNFDL
AEDAKLQLSKDTYDDDLDNLLAQIGDQYADLFLAAKNLSDAILLSDILRVNTEITKAPL
SASMIKRYDEHHQDLTLLKALVRQQLPEKYKEIFFDQSKNGYAGYIDGGASQEEFYKFI
KPILEKMDGTEELLVKLNREDLLRKQRTFDNGSIPHQIHLGELHAILRRQEDFYPFLKD
NREKIEKILTFRIPYYVGPLARGNSRFAWMTRKSEETITPWNFEEVVDKGASAQSFIER
MTNFDKNLPNEKVLPKHSLLYEYFTVYNELTKVKYVTEGMRKPAFLSGEQKKAIVDLLF
KTNRKVTVKQLKEDYFKKIECFDSVEISGVEDRFNASLGTYHDLLKIIKDKDFLDNEEN
EDILEDIVLTLTLFEDREMIEERLKTYAHLFDDKVMKQLKRRRYTGWGRLSRKLINGIR
DKQSGKTILDFLKSDGFANRNFMQLIHDDSLTFKEDIQKAQVSGQGDSLHEHIANLAGS
PAIKKGILQTVKVVDELVKVMGRHKPENIVIEMARENQTTQKGQKNSRERMKRIEEGIK
ELGSQILKEHPVENTQLQNEKLYLYYLQNGRDMYVDQELDINRLSDYDVDHIVPQSFLK
DDSIDNKVLTRSDKNRGKSDNVPSEEVVKKMKNYWRQLLNAKLITQRKFDNLTKAERGG
LSELDKAGFIKRQLVETRQITKHVAQILDSRMNTKYDENDKLIREVKVITLKSKLVSDF
RKDFQFYKVREINNYHHAHDAYLNAVVGTALIKKYPKLESEFVYGDYKVYDVRKMIAKS
EQEIGKATAKYFFYSNIMNFFKTEITLANGEIRKRPLIETNGETGEIVWDKGRDFATVR
KVLSMPQVNIVKKTEVQTGGFSKESILPKRNSDKLIARKKDWDPKKYGGFDSPTVAYSV
LVVAKVEKGKSKKLKSVKELLGITIMERSSFEKNPIDFLEAKGYKEVKKDLIIKLPKYS
LFELENGRKRMLASAGELQKGNELALPSKYVNFLYLASHYEKLKGSPEDNEQKQLFVEQ
HKHYLDEIIEQISEFSKRVILADANLDKVLSAYNKHRDKPIREQAENIIHLFTLTNLGA
PAAFKYFDTTIDRKRYTSTKEVLDATLIHQSITGLYETRIDLSQLGGDSGGSANELTWH
DVLAEEKQQPYFLNTLQTVASERQSGVTIYPPQKDVFNAFRFTELGDVKVVILGQDPYH
GPGQAHGLAFSVRPGIAIPPSLLNMYKELENTIPGFTRPNHGYLESWARQGVLLLNTVL
TVRAGQAHSHASLGWETFTDKVISLINQHREGVVFLLWGSHAQKKGAIIDKQRHHVLKA
PHPSPLSAHRGFFGCNHFVLANQWLEQRGETPIDWMPVLPAESESGGSPKKKRKVGGGG
SGATNFSLLKQAGDVEENPGPMVSKGEELFTGVVPILVELDGDVNGHKFSVSGEGEGDA
TYGKLTLKFICTTGKLPVPWPTLVTTLTYGVQCFSRYPDHMKQHDFFKSAMPEGYVQER
TIFFKDDGNYKTRAEVKFEGDTLVNRIELKGIDFKEDGNILGHKLEYNYNSHNVYIMAD
KQKNGIKVNFKIRHNIEDGSVQLADHYQQNTPIGDGPVLLPDNHYLSTQSALSKDPNEK
RDHMVLLEFVTAAGITLGMDELYK
BE4max(R33A) w/o UGI = miniCGBEl, SEQ ID NO: 427
MKRTADGSEFESPKKKRKVSSETGPVAVDPTLRRRIEPHEFEVFFDPRELAKETCLLYE
INWGGRHSIWRHTSQNTNKHVEVNFIEKFTTERYFCPNTRCSITWFLSWSPCGECSRAI
TEFLSRYPHVTLFIYIARLYHHADPRNRQGLRDLISSGVTIQIMTEQESGYCWRNFVNY
SPSNEAHWPRYPHLWVRLYVLELYCIILGLPPCLNILRRKQPQLTFFTIALQSCHYQRL
PPHILWATGLKSGGSSGGSSGSETPGTSESATPESSGGSSGGSDKKYSIGLAIGTNSVG
WAVITDEYKVPSKKFKVLGNTDRHSIKKNLIGALLFDSGETAEATRLKRTARRRYTRRK
NRICYLQEIFSNEMAKVDDSFFHRLEESFLVEEDKKHERHPIFGNIVDEVAYHEKYPTI
YHLRKKLVDSTDKADLRLIYLALAHMIKFRGHFLIEGDLNPDNSDVDKLFIQLVQTYNQ
LFEENPINASGVDAKAILSARLSKSRRLENLIAQLPGEKKNGLFGNLIALSLGLTPNFK
SNFDLAEDAKLQLSKDTYDDDLDNLLAQIGDQYADLFLAAKNLSDAILLSDILRVNTEI
TKAPLSASMIKRYDEHHQDLTLLKALVRQQLPEKYKEIFFDQSKNGYAGYIDGGASQEE
FYKFIKPILEKMDGTEELLVKLNREDLLRKQRTFDNGSIPHQIHLGELHAILRRQEDFY
PFLKDNREKIEKILTFRIPYYVGPLARGNSRFAWMTRKSEETITPWNFEEVVDKGASAQ
SFIERMTNFDKNLPNEKVLPKHSLLYEYFTVYNELTKVKYVTEGMRKPAFLSGEQKKAI
VDLLFKTNRKVTVKQLKEDYFKKIECFDSVEISGVEDRFNASLGTYHDLLKIIKDKDFL
DNEENEDILEDIVLTLTLFEDREMIEERLKTYAHLFDDKVMKQLKRRRYTGWGRLSRKL
INGIRDKQSGKTILDFLKSDGFANRNFMQLIHDDSLTFKEDIQKAQVSGQGDSLHEHIA
NLAGSPAIKKGILQTVKVVDELVKVMGRHKPENIVIEMARENQTTQKGQKNSRERMKRI
EEGIKELGSQILKEHPVENTQLQNEKLYLYYLQNGRDMYVDQELDINRLSDYDVDHIVP
QSFLKDDSIDNKVLTRSDKNRGKSDNVPSEEVVKKMKNYWRQLLNAKLITQRKFDNLTK
AERGGLSELDKAGFIKRQLVETRQITKHVAQILDSRMNTKYDENDKLIREVKVITLKSK
LVSDFRKDFQFYKVREINNYHHAHDAYLNAVVGTALIKKYPKLESEFVYGDYKVYDVRK
MIAKSEQEIGKATAKYFFYSNIMNFFKTEITLANGEIRKRPLIETNGETGEIVWDKGRD
FATVRKVLSMPQVNIVKKTEVQTGGFSKESILPKRNSDKLIARKKDWDPKKYGGFDSPT
VAYSVLVVAKVEKGKSKKLKSVKELLGITIMERSSFEKNPIDFLEAKGYKEVKKDLIIK
LPKYSLFELENGRKRMLASAGELQKGNELALPSKYVNFLYLASHYEKLKGSPEDNEQKQ
LFVEQHKHYLDEIIEQISEFSKRVILADANLDKVLSAYNKHRDKPIREQAENIIHLFTL
TNLGAPAAFKYFDTTIDRKRYTSTKEVLDATLIHQSITGLYETRIDLSQLGGDSGGSKR
TADGSEFEPKKKRKVGGGGSGATNFSLLKQAGDVEENPGPMVSKGEELFTGVVPILVEL
DGDVNGHKFSVSGEGEGDATYGKLTLKFICTTGKLPVPWPTLVTTLTYGVQCFSRYPDH
MKQHDFFKSAMPEGYVQERTIFFKDDGNYKTRAEVKFEGDTLVNRIELKGIDFKEDGNI
LGHKLEYNYNSHNVYIMADKQKNGIKVNFKIRHNIEDGSVQLADHYQQNTPIGDGPVLL
PDNHYLSTQSALSKDPNEKRDHMVLLEFVTAAGITLGMDELYK
CGBE1_NG, SEQ ID NO: 428
MKRTADGSEFESPKKKRKVANELTWHDVLAEEKQQPYFLNTLQTVASERQSGVTIYPPQ
KDVFNAFRFTELGDVKVVILGQDPYHGPGQAHGLAFSVRPGIAIPPSLLNMYKELENTI
PGFTRPNHGYLESWARQGVLLLNTVLTVRAGQAHSHASLGWETFTDKVISLINQHREGV
VFLLWGSHAQKKGAIIDKQRHHVLKAPHPSPLSAHRGFFGCNHFVLANQWLEQRGETPI
DWMPVLPAESESGGSGGSGGSSSETGPVAVDPTLRRRIEPHEFEVFFDPRELAKETCLL
YEINWGGRHSIWRHTSQNTNKHVEVNFIEKFTTERYFCPNTRCSITWFLSWSPCGECSR
AITEFLSRYPHVTLFIYIARLYHHADPRNRQGLRDLISSGVTIQIMTEQESGYCWRNFV
NYSPSNEAHWPRYPHLWVRLYVLELYCIILGLPPCLNILRRKQPQLTFFTIALQSCHYQ
RLPPHILWATGLKSGGSSGGSSGSETPGTSESATPESSGGSSGGSDKKYSIGLAIGTNS
VGWAVITDEYKVPSKKFKVLGNTDRHSIKKNLIGALLFDSGETAEATRLKRTARRRYTR
RKNRICYLQEIFSNEMAKVDDSFFHRLEESFLVEEDKKHERHPIFGNIVDEVAYHEKYP
TIYHLRKKLVDSTDKADLRLIYLALAHMIKFRGHFLIEGDLNPDNSDVDKLFIQLVQTY
NQLFEENPINASGVDAKAILSARLSKSRRLENLIAQLPGEKKNGLFGNLIALSLGLTPN
FKSNFDLAEDAKLQLSKDTYDDDLDNLLAQIGDQYADLFLAAKNLSDAILLSDILRVNT
EITKAPLSASMIKRYDEHHQDLTLLKALVRQQLPEKYKEIFFDQSKNGYAGYIDGGASQ
EEFYKFIKPILEKMDGTEELLVKLNREDLLRKQRTFDNGSIPHQIHLGELHAILRRQED
FYPFLKDNREKIEKILTFRIPYYVGPLARGNSRFAWMTRKSEETITPWNFEEVVDKGAS
AQSFIERMTNFDKNLPNEKVLPKHSLLYEYFTVYNELTKVKYVTEGMRKPAFLSGEQKK
AIVDLLFKTNRKVTVKQLKEDYFKKIECFDSVEISGVEDRFNASLGTYHDLLKIIKDKD
FLDNEENEDILEDIVLTLTLFEDREMIEERLKTYAHLFDDKVMKQLKRRRYTGWGRLSR
KLINGIRDKQSGKTILDFLKSDGFANRNFMQLIHDDSLTFKEDIQKAQVSGQGDSLHEH
IANLAGSPAIKKGILQTVKVVDELVKVMGRHKPENIVIEMARENQTTQKGQKNSRERMK
RIEEGIKELGSQILKEHPVENTQLQNEKLYLYYLQNGRDMYVDQELDINRLSDYDVDHI
VPQSFLKDDSIDNKVLTRSDKNRGKSDNVPSEEVVKKMKNYWRQLLNAKLITQRKFDNL
TKAERGGLSELDKAGFIKRQLVETRQITKHVAQILDSRMNTKYDENDKLIREVKVITLK
SKLVSDFRKDFQFYKVREINNYHHAHDAYLNAVVGTALIKKYPKLESEFVYGDYKVYDV
RKMIAKSEQEIGKATAKYFFYSNIMNFFKTEITLANGEIRKRPLIETNGETGEIVWDKG
RDFATVRKVLSMPQVNIVKKTEVQTGGFSKESIRPKRNSDKLIARKKDWDPKKYGGFVS
PTVAYSVLVVAKVEKGKSKKLKSVKELLGITIMERSSFEKNPIDFLEAKGYKEVKKDLI
IKLPKYSLFELENGRKRMLASARFLQKGNELALPSKYVNFLYLASHYEKLKGSPEDNEQ
KQLFVEQHKHYLDEIIEQISEFSKRVILADANLDKVLSAYNKHRDKPIREQAENIIHLF
TLTNLGAPRAFKYFDTTIDRKVYRSTKEVLDATLIHQSITGLYETRIDLSQLGGDSGGS
KRTADGSEFEPKKKRKVGGGGSGATNFSLLKQAGDVEENPGPMVSKGEELFTGVVPILV
ELDGDVNGHKFSVSGEGEGDATYGKLTLKFICTTGKLPVPWPTLVTTLTYGVQCFSRYP
DHMKQHDFFKSAMPEGYVQERTIFFKDDGNYKTRAEVKFEGDTLVNRIELKGIDFKEDG
NILGHKLEYNYNSHNVYIMADKQKNGIKVNFKIRHNIEDGSVQLADHYQQNTPIGDGPV
LLPDNHYLSTQSALSKDPNEKRDHMVLLEFVTAAGITLGMDELYK
miniCGBE1_NG, SEQ ID NO: 429
MKRTADGSEFESPKKKRKVSSETGPVAVDPTLRRRIEPHEFEVFFDPRELAKETCLLYE
INWGGRHSIWRHTSQNTNKHVEVNFIEKFTTERYFCPNTRCSITWFLSWSPCGECSRAI
TEFLSRYPHVTLFIYIARLYHHADPRNRQGLRDLISSGVTIQIMTEQESGYCWRNFVNY
SPSNEAHWPRYPHLWVRLYVLELYCIILGLPPCLNILRRKQPQLTFFTIALQSCHYQRL
PPHILWATGLKSGGSSGGSSGSETPGTSESATPESSGGSSGGSDKKYSIGLAIGTNSVG
WAVITDEYKVPSKKFKVLGNTDRHSIKKNLIGALLFDSGETAEATRLKRTARRRYTRRK
NRICYLQEIFSNEMAKVDDSFFHRLEESFLVEEDKKHERHPIFGNIVDEVAYHEKYPTI
YHLRKKLVDSTDKADLRLIYLALAHMIKFRGHFLIEGDLNPDNSDVDKLFIQLVQTYNQ
LFEENPINASGVDAKAILSARLSKSRRLENLIAQLPGEKKNGLFGNLIALSLGLTPNFK
SNFDLAEDAKLQLSKDTYDDDLDNLLAQIGDQYADLFLAAKNLSDAILLSDILRVNTEI
TKAPLSASMIKRYDEHHQDLTLLKALVRQQLPEKYKEIFFDQSKNGYAGYIDGGASQEE
FYKFIKPILEKMDGTEELLVKLNREDLLRKQRTFDNGSIPHQIHLGELHAILRRQEDFY
PFLKDNREKIEKILTFRIPYYVGPLARGNSRFAWMTRKSEETITPWNFEEVVDKGASAQ
SFIERMTNFDKNLPNEKVLPKHSLLYEYFTVYNELTKVKYVTEGMRKPAFLSGEQKKAI
VDLLFKTNRKVTVKQLKEDYFKKIECFDSVEISGVEDRFNASLGTYHDLLKIIKDKDFL
DNEENEDILEDIVLTLTLFEDREMIEERLKTYAHLFDDKVMKQLKRRRYTGWGRLSRKL
INGIRDKQSGKTILDFLKSDGFANRNFMQLIHDDSLTFKEDIQKAQVSGQGDSLHEHIA
NLAGSPAIKKGILQTVKVVDELVKVMGRHKPENIVIEMARENQTTQKGQKNSRERMKRI
EEGIKELGSQILKEHPVENTQLQNEKLYLYYLQNGRDMYVDQELDINRLSDYDVDHIVP
QSFLKDDSIDNKVLTRSDKNRGKSDNVPSEEVVKKMKNYWRQLLNAKLITQRKFDNLTK
AERGGLSELDKAGFIKRQLVETRQITKHVAQILDSRMNTKYDENDKLIREVKVITLKSK
LVSDFRKDFQFYKVREINNYHHAHDAYLNAVVGTALIKKYPKLESEFVYGDYKVYDVRK
MIAKSEQEIGKATAKYFFYSNIMNFFKTEITLANGEIRKRPLIETNGETGEIVWDKGRD
FATVRKVLSMPQVNIVKKTEVQTGGFSKESIRPKRNSDKLIARKKDWDPKKYGGFVSPT
VAYSVLVVAKVEKGKSKKLKSVKELLGITIMERSSFEKNPIDFLEAKGYKEVKKDLIIK
LPKYSLFELENGRKRMLASARFLQKGNELALPSKYVNFLYLASHYEKLKGSPEDNEQKQ
LFVEQHKHYLDEIIEQISEFSKRVILADANLDKVLSAYNKHRDKPIREQAENIIHLFTL
TNLGAPRAFKYFDTTIDRKVYRSTKEVLDATLIHQSITGLYETRIDLSQLGGDSGGSKR
TADGSEFEPKKKRKVGGGGSGATNFSLLKQAGDVEENPGPMVSKGEELFTGVVPILVEL
DGDVNGHKFSVSGEGEGDATYGKLTLKFICTTGKLPVPWPTLVTTLTYGVQCFSRYPDH
MKQHDFFKSAMPEGYVQERTIFFKDDGNYKTRAEVKFEGDTLVNRIELKGIDFKEDGNI
LGHKLEYNYNSHNVYIMADKQKNGIKVNFKIRHNIEDGSVQLADHYQQNTPIGDGPVLL
PDNHYLSTQSALSKDPNEKRDHMVLLEFVTAAGITLGMDELYK
CGBE1_VRQR, SEQ ID NO: 430
MKRTADGSEFESPKKKRKVANELTWHDVLAEEKQQPYFLNTLQTVASERQSGVTIYPPQ
KDVFNAFRFTELGDVKVVILGQDPYHGPGQAHGLAFSVRPGIAIPPSLLNMYKELENTI
PGFTRPNHGYLESWARQGVLLLNTVLTVRAGQAHSHASLGWETFTDKVISLINQHREGV
VFLLWGSHAQKKGAIIDKQRHHVLKAPHPSPLSAHRGFFGCNHFVLANQWLEQRGETPI
DWMPVLPAESESGGSGGSGGSSSETGPVAVDPTLRRRIEPHEFEVFFDPRELAKETCLL
YEINWGGRHSIWRHTSQNTNKHVEVNFIEKFTTERYFCPNTRCSITWFLSWSPCGECSR
AITEFLSRYPHVTLFIYIARLYHHADPRNRQGLRDLISSGVTIQIMTEQESGYCWRNFV
NYSPSNEAHWPRYPHLWVRLYVLELYCIILGLPPCLNILRRKQPQLTFFTIALQSCHYQ
RLPPHILWATGLKSGGSSGGSSGSETPGTSESATPESSGGSSGGSDKKYSIGLAIGTNS
VGWAVITDEYKVPSKKFKVLGNTDRHSIKKNLIGALLFDSGETAEATRLKRTARRRYTR
RKNRICYLQEIFSNEMAKVDDSFFHRLEESFLVEEDKKHERHPIFGNIVDEVAYHEKYP
TIYHLRKKLVDSTDKADLRLIYLALAHMIKFRGHFLIEGDLNPDNSDVDKLFIQLVQTY
NQLFEENPINASGVDAKAILSARLSKSRRLENLIAQLPGEKKNGLFGNLIALSLGLTPN
FKSNFDLAEDAKLQLSKDTYDDDLDNLLAQIGDQYADLFLAAKNLSDAILLSDILRVNT
EITKAPLSASMIKRYDEHHQDLTLLKALVRQQLPEKYKEIFFDQSKNGYAGYIDGGASQ
EEFYKFIKPILEKMDGTEELLVKLNREDLLRKQRTFDNGSIPHQIHLGELHAILRRQED
FYPFLKDNREKIEKILTFRIPYYVGPLARGNSRFAWMTRKSEETITPWNFEEVVDKGAS
AQSFIERMTNFDKNLPNEKVLPKHSLLYEYFTVYNELTKVKYVTEGMRKPAFLSGEQKK
AIVDLLFKTNRKVTVKQLKEDYFKKIECFDSVEISGVEDRFNASLGTYHDLLKIIKDKD
FLDNEENEDILEDIVLTLTLFEDREMIEERLKTYAHLFDDKVMKQLKRRRYTGWGRLSR
KLINGIRDKQSGKTILDFLKSDGFANRNFMQLIHDDSLTFKEDIQKAQVSGQGDSLHEH
IANLAGSPAIKKGILQTVKVVDELVKVMGRHKPENIVIEMARENQTTQKGQKNSRERMK
RIEEGIKELGSQILKEHPVENTQLQNEKLYLYYLQNGRDMYVDQELDINRLSDYDVDHI
VPQSFLKDDSIDNKVLTRSDKNRGKSDNVPSEEVVKKMKNYWRQLLNAKLITQRKFDNL
TKAERGGLSELDKAGFIKRQLVETRQITKHVAQILDSRMNTKYDENDKLIREVKVITLK
SKLVSDFRKDFQFYKVREINNYHHAHDAYLNAVVGTALIKKYPKLESEFVYGDYKVYDV
RKMIAKSEQEIGKATAKYFFYSNIMNFFKTEITLANGEIRKRPLIETNGETGEIVWDKG
RDFATVRKVLSMPQVNIVKKTEVQTGGFSKESILPKRNSDKLIARKKDWDPKKYGGFVS
PTVAYSVLVVAKVEKGKSKKLKSVKELLGITIMERSSFEKNPIDFLEAKGYKEVKKDLI
IKLPKYSLFELENGRKRMLASARELQKGNELALPSKYVNFLYLASHYEKLKGSPEDNEQ
KQLFVEQHKHYLDEIIEQISEFSKRVILADANLDKVLSAYNKHRDKPIREQAENIIHLF
TLTNLGAPAAFKYFDTTIDRKQYRSTKEVLDATLIHQSITGLYETRIDLSQLGGDSGGS
KRTADGSEFEPKKKRKVGGGGSGATNFSLLKQAGDVEENPGPMVSKGEELFTGVVPILV
ELDGDVNGHKFSVSGEGEGDATYGKLTLKFICTTGKLPVPWPTLVTTLTYGVQCFSRYP
DHMKQHDFFKSAMPEGYVQERTIFFKDDGNYKTRAEVKFEGDTLVNRIELKGIDFKEDG
NILGHKLEYNYNSHNVYIMADKQKNGIKVNFKIRHNIEDGSVQLADHYQQNTPIGDGPV
LLPDNHYLSTQSALSKDPNEKRDHMVLLEFVTAAGITLGMDELYK
miniCGBE1_VRQR, SEQ ID NO: 431
MKRTADGSEFESPKKKRKVSSETGPVAVDPTLRRRIEPHEFEVFFDPRELAKETCLLYE
INWGGRHSIWRHTSQNTNKHVEVNFIEKFTTERYFCPNTRCSITWFLSWSPCGECSRAI
TEFLSRYPHVTLFIYIARLYHHADPRNRQGLRDLISSGVTIQIMTEQESGYCWRNFVNY
SPSNEAHWPRYPHLWVRLYVLELYCIILGLPPCLNILRRKQPQLTFFTIALQSCHYQRL
PPHILWATGLKSGGSSGGSSGSETPGTSESATPESSGGSSGGSDKKYSIGLAIGTNSVG
WAVITDEYKVPSKKFKVLGNTDRHSIKKNLIGALLFDSGETAEATRLKRTARRRYTRRK
NRICYLQEIFSNEMAKVDDSFFHRLEESFLVEEDKKHERHPIFGNIVDEVAYHEKYPTI
YHLRKKLVDSTDKADLRLIYLALAHMIKFRGHFLIEGDLNPDNSDVDKLFIQLVQTYNQ
LFEENPINASGVDAKAILSARLSKSRRLENLIAQLPGEKKNGLFGNLIALSLGLTPNFK
SNFDLAEDAKLQLSKDTYDDDLDNLLAQIGDQYADLFLAAKNLSDAILLSDILRVNTEI
TKAPLSASMIKRYDEHHQDLTLLKALVRQQLPEKYKEIFFDQSKNGYAGYIDGGASQEE
FYKFIKPILEKMDGTEELLVKLNREDLLRKQRTFDNGSIPHQIHLGELHAILRRQEDFY
PFLKDNREKIEKILTFRIPYYVGPLARGNSRFAWMTRKSEETITPWNFEEVVDKGASAQ
SFIERMTNFDKNLPNEKVLPKHSLLYEYFTVYNELTKVKYVTEGMRKPAFLSGEQKKAI
VDLLFKTNRKVTVKQLKEDYFKKIECFDSVEISGVEDRFNASLGTYHDLLKIIKDKDFL
DNEENEDILEDIVLTLTLFEDREMIEERLKTYAHLFDDKVMKQLKRRRYTGWGRLSRKL
INGIRDKQSGKTILDFLKSDGFANRNFMQLIHDDSLTFKEDIQKAQVSGQGDSLHEHIA
NLAGSPAIKKGILQTVKVVDELVKVMGRHKPENIVIEMARENQTTQKGQKNSRERMKRI
EEGIKELGSQILKEHPVENTQLQNEKLYLYYLQNGRDMYVDQELDINRLSDYDVDHIVP
QSFLKDDSIDNKVLTRSDKNRGKSDNVPSEEVVKKMKNYWRQLLNAKLITQRKFDNLTK
AERGGLSELDKAGFIKRQLVETRQITKHVAQILDSRMNTKYDENDKLIREVKVITLKSK
LVSDFRKDFQFYKVREINNYHHAHDAYLNAVVGTALIKKYPKLESEFVYGDYKVYDVRK
MIAKSEQEIGKATAKYFFYSNIMNFFKTEITLANGEIRKRPLIETNGETGEIVWDKGRD
FATVRKVLSMPQVNIVKKTEVQTGGFSKESILPKRNSDKLIARKKDWDPKKYGGFVSPT
VAYSVLVVAKVEKGKSKKLKSVKELLGITIMERSSFEKNPIDFLEAKGYKEVKKDLIIK
LPKYSLFELENGRKRMLASARELQKGNELALPSKYVNFLYLASHYEKLKGSPEDNEQKQ
LFVEQHKHYLDEIIEQISEFSKRVILADANLDKVLSAYNKHRDKPIREQAENIIHLFTL
TNLGAPAAFKYFDTTIDRKQYRSTKEVLDATLIHQSITGLYETRIDLSQLGGDSGGSKR
TADGSEFEPKKKRKVGGGGSGATNFSLLKQAGDVEENPGPMVSKGEELFTGVVPILVEL
DGDVNGHKFSVSGEGEGDATYGKLTLKFICTTGKLPVPWPTLVTTLTYGVQCFSRYPDH
MKQHDFFKSAMPEGYVQERTIFFKDDGNYKTRAEVKFEGDTLVNRIELKGIDFKEDGNI
LGHKLEYNYNSHNVYIMADKQKNGIKVNFKIRHNIEDGSVQLADHYQQNTPIGDGPVLL
PDNHYLSTQSALSKDPNEKRDHMVLLEFVTAAGITLGMDELYK
Prime Editor 1 (PE1), SEQ ID NO: 432
MKRTADGSEFESPKKKRKVDKKYSIGLDIGTNSVGWAVITDEYKVPSKKFKVLGNTDRH
SIKKNLIGALLFDSGETAEATRLKRTARRRYTRRKNRICYLQEIFSNEMAKVDDSFFHR
LEESFLVEEDKKHERHPIFGNIVDEVAYHEKYPTIYHLRKKLVDSTDKADLRLIYLALA
HMIKFRGHFLIEGDLNPDNSDVDKLFIQLVQTYNQLFEENPINASGVDAKAILSARLSK
SRRLENLIAQLPGEKKNGLFGNLIALSLGLTPNFKSNFDLAEDAKLQLSKDTYDDDLDN
LLAQIGDQYADLFLAAKNLSDAILLSDILRVNTEITKAPLSASMIKRYDEHHQDLTLLK
ALVRQQLPEKYKEIFFDQSKNGYAGYIDGGASQEEFYKFIKPILEKMDGTEELLVKLNR
EDLLRKQRTFDNGSIPHQIHLGELHAILRRQEDFYPFLKDNREKIEKILTFRIPYYVGP
LARGNSRFAWMTRKSEETITPWNFEEVVDKGASAQSFIERMTNFDKNLPNEKVLPKHSL
LYEYFTVYNELTKVKYVTEGMRKPAFLSGEQKKAIVDLLFKTNRKVTVKQLKEDYFKKI
ECFDSVEISGVEDRFNASLGTYHDLLKIIKDKDFLDNEENEDILEDIVLTLTLFEDREM
IEERLKTYAHLFDDKVMKQLKRRRYTGWGRLSRKLINGIRDKQSGKTILDFLKSDGFAN
RNFMQLIHDDSLTFKEDIQKAQVSGQGDSLHEHIANLAGSPAIKKGILQTVKVVDELVK
VMGRHKPENIVIEMARENQTTQKGQKNSRERMKRIEEGIKELGSQILKEHPVENTQLQN
EKLYLYYLQNGRDMYVDQELDINRLSDYDVDAIVPQSFLKDDSIDNKVLTRSDKNRGKS
DNVPSEEVVKKMKNYWRQLLNAKLITQRKFDNLTKAERGGLSELDKAGFIKRQLVETRQ
ITKHVAQILDSRMNTKYDENDKLIREVKVITLKSKLVSDFRKDFQFYKVREINNYHHAH
DAYLNAVVGTALIKKYPKLESEFVYGDYKVYDVRKMIAKSEQEIGKATAKYFFYSNIMN
FFKTEITLANGEIRKRPLIETNGETGEIVWDKGRDFATVRKVLSMPQVNIVKKTEVQTG
GFSKESILPKRNSDKLIARKKDWDPKKYGGFDSPTVAYSVLVVAKVEKGKSKKLKSVKE
LLGITIMERSSFEKNPIDFLEAKGYKEVKKDLIIKLPKYSLFELENGRKRMLASAGELQ
KGNELALPSKYVNFLYLASHYEKLKGSPEDNEQKQLFVEQHKHYLDEIIEQISEFSKRV
ILADANLDKVLSAYNKHRDKPIREQAENIIHLFTLTNLGAPAAFKYFDTTIDRKRYTST
KEVLDATLIHQSITGLYETRIDLSQLGGDSGGSSGGSSGSETPGTSESATPESSGGSSG
GSSTLNIEDEYRLHETSKEPDVSLGSTWLSDFPQAWAETGGMGLAVRQAPLIIPLKATS
TPVSIKQYPMSQEARLGIKPHIQRLLDQGILVPCQSPWNTPLLPVKKPGTNDYRPVQDL
REVNKRVEDIHPTVPNPYNLLSGLPPSHQWYTVLDLKDAFFCLRLHPTSQPLFAFEWRD
PEMGISGQLTWTRLPQGFKNSPTLFDEALHRDLADFRIQHPDLILLQYVDDLLLAATSE
LDCQQGTRALLQTLGNLGYRASAKKAQICQKQVKYLGYLLKEGQRWLTEARKETVMGQP
TPKTPRQLREFLGTAGFCRLWIPGFAEMAAPLYPLTKTGTLFNWGPDQQKAYQEIKQAL
LTAPALGLPDLTKPFELFVDEKQGYAKGVLTQKLGPWRRPVAYLSKKLDPVAAGWPPCL
RMVAAIAVLTKDAGKLTMGQPLVILAPHAVEALVKQPPDRWLSNARMTHYQALLLDTDR
VQFGPVVALNPATLLPLPEEGLQHNCLDILAEAHGTRPDLTDQPLPDADHTWYTDGSSL
LQEGQRKAGAAVTTETEVIWAKALPAGTSAQRAELIALTQALKMAEGKKLNVYTDSRYA
FATAHIHGEIYRRRGLLTSEGKEIKNKDEILALLKALFLPKRLSIIHCPGHQKGHSAEA
RGNRMADQAARKAAITETPDTSTLLIENSSPSGGSKRTADGSEFEPKKKRKV
Prime Editor 2 (PE2), SEQ ID NO: 433
MKRTADGSEFESPKKKRKVDKKYSIGLDIGTNSVGWAVITDEYKVPSKKFKVLGNTDRH
SIKKNLIGALLFDSGETAEATRLKRTARRRYTRRKNRICYLQEIFSNEMAKVDDSFFHR
LEESFLVEEDKKHERHPIFGNIVDEVAYHEKYPTIYHLRKKLVDSTDKADLRLIYLALA
HMIKFRGHFLIEGDLNPDNSDVDKLFIQLVQTYNQLFEENPINASGVDAKAILSARLSK
SRRLENLIAQLPGEKKNGLFGNLIALSLGLTPNFKSNFDLAEDAKLQLSKDTYDDDLDN
LLAQIGDQYADLFLAAKNLSDAILLSDILRVNTEITKAPLSASMIKRYDEHHQDLTLLK
ALVRQQLPEKYKEIFFDQSKNGYAGYIDGGASQEEFYKFIKPILEKMDGTEELLVKLNR
EDLLRKQRTFDNGSIPHQIHLGELHAILRRQEDFYPFLKDNREKIEKILTFRIPYYVGP
LARGNSRFAWMTRKSEETITPWNFEEVVDKGASAQSFIERMTNFDKNLPNEKVLPKHSL
LYEYFTVYNELTKVKYVTEGMRKPAFLSGEQKKAIVDLLFKTNRKVTVKQLKEDYFKKI
ECFDSVEISGVEDRFNASLGTYHDLLKIIKDKDFLDNEENEDILEDIVLTLTLFEDREM
IEERLKTYAHLFDDKVMKQLKRRRYTGWGRLSRKLINGIRDKQSGKTILDFLKSDGFAN
RNFMQLIHDDSLTFKEDIQKAQVSGQGDSLHEHIANLAGSPAIKKGILQTVKVVDELVK
VMGRHKPENIVIEMARENQTTQKGQKNSRERMKRIEEGIKELGSQILKEHPVENTQLQN
EKLYLYYLQNGRDMYVDQELDINRLSDYDVDAIVPQSFLKDDSIDNKVLTRSDKNRGKS
DNVPSEEVVKKMKNYWRQLLNAKLITQRKFDNLTKAERGGLSELDKAGFIKRQLVETRQ
ITKHVAQILDSRMNTKYDENDKLIREVKVITLKSKLVSDFRKDFQFYKVREINNYHHAH
DAYLNAVVGTALIKKYPKLESEFVYGDYKVYDVRKMIAKSEQEIGKATAKYFFYSNIMN
FFKTEITLANGEIRKRPLIETNGETGEIVWDKGRDFATVRKVLSMPQVNIVKKTEVQTG
GFSKESILPKRNSDKLIARKKDWDPKKYGGFDSPTVAYSVLVVAKVEKGKSKKLKSVKE
LLGITIMERSSFEKNPIDFLEAKGYKEVKKDLIIKLPKYSLFELENGRKRMLASAGELQ
KGNELALPSKYVNFLYLASHYEKLKGSPEDNEQKQLFVEQHKHYLDEIIEQISEFSKRV
ILADANLDKVLSAYNKHRDKPIREQAENIIHLFTLTNLGAPAAFKYFDTTIDRKRYTST
KEVLDATLIHQSITGLYETRIDLSQLGGDSGGSSGGSSGSETPGTSESATPESSGGSSG
GSSTLNIEDEYRLHETSKEPDVSLGSTWLSDFPQAWAETGGMGLAVRQAPLIIPLKATS
TPVSIKQYPMSQEARLGIKPHIQRLLDQGILVPCQSPWNTPLLPVKKPGTNDYRPVQDL
REVNKRVEDIHPTVPNPYNLLSGLPPSHQWYTVLDLKDAFFCLRLHPTSQPLFAFEWRD
PEMGISGQLTWTRLPQGFKNSPTLFNEALHRDLADFRIQHPDLILLQYVDDLLLAATSE
LDCQQGTRALLQTLGNLGYRASAKKAQICQKQVKYLGYLLKEGQRWLTEARKETVMGQP
TPKTPRQLREFLGKAGFCRLFIPGFAEMAAPLYPLTKPGTLFNWGPDQQKAYQEIKQAL
LTAPALGLPDLTKPFELFVDEKQGYAKGVLTQKLGPWRRPVAYLSKKLDPVAAGWPPCL
RMVAAIAVLTKDAGKLTMGQPLVILAPHAVEALVKQPPDRWLSNARMTHYQALLLDTDR
VQFGPVVALNPATLLPLPEEGLQHNCLDILAEAHGTRPDLTDQPLPDADHTWYTDGSSL
LQEGQRKAGAAVTTETEVIWAKALPAGTSAQRAELIALTQALKMAEGKKLNVYTDSRYA
FATAHIHGEIYRRRGWLTSEGKEIKNKDEILALLKALFLPKRLSIIHCPGHQKGHSAEA
RGNRMADQAARKAAITETPDTSTLLIENSSPSGGSKRTADGSEFEPKKKRKV
CAST system, CRISPR-associated transposase
from cyanobacteria Scytonema hofmanni-
ShCas12k (WP_029636312.1) SEQ ID NO: 434
MSQITIQARLISFESNRQQLWKLMADLNTPLINELLCQLGQHPDFEKWQQKGKLPSTVV
SQLCQPLKTDPRFAGQPSRLYMSAIHIVDYIYKSWLAIQKRLQQQLDGKTRWLEMLNSD
AELVELSGDTLEAIRVKAAEILAIAMPASESDSASPKGKKGKKEKKPSSSSPKRSLSKT
LFDAYQETEDIKSRSAISYLLKNGCKLTDKEEDSEKFAKRRRQVEIQIQRLTEKLISRM
PKGRDLTNAKWLETLLTATTTVAEDNAQAKRWQDILLTRSSSLPFPLVFETNEDMVWSK
NQKGRLCVHFNGLSDLIFEVYCGNRQLHWFQRFLEDQQTKRKSKNQHSSGLFTLRNGHL
VWLEGEGKGEPWNLHHLTLYCCVDNRLWTEEGTEIVRQEKADEITKFITNMKKKSDLSD
TQQALIQRKQSTLTRINNSFERPSQPLYQGQSHILVGVSLGLEKPATVAVVDAIANKVL
AYRSIKQLLGDNYELLNRQRRQQQYLSHERHKAQKNFSPNQFGASELGQHIDRLLAKAI
VALARTYKAGSIVLPKLGDMREVVQSEIQAIAEQKFPGYIEGQQKYAKQYRVNVHRWSY
GRLIQSIQSKAAQTGIVIEEGKQPIRGSPHDKAKELALSAYNLRLTRRS
CAST system, CRISPR-associated transposase
from cyanobacteria Scytonema hofmanni-
ShTniQ -- WP_029636334.1 SEQ ID NO: 435
MIEAPDVKPWLFLIKPYEGESLSHFLGRFRRANHLSASGLGTLAGIGAIVARWERFHFN
PRPSQQELEAIASVVEVDAQRLAQMLPPAGVGMQHEPIRLCGACYAESPCHRIEWQYKS
VWKCDRHQLKILAKCPNCQAPFKMPALWEDGCCHRCRMPFAEMAKLQKV
CAST system, CRISPR-associated transposase
from cyanobacteria Scytonema hofmanni-
ShTnsB -- WP_084763316.1 SEQ ID NO: 436
MNSQQNPDLAVHPLAIPMEGLLGESATTLEKNVIATQLSEEAQVKLEVIQSLLEPCDRT
TYGQKLREAAEKLNVSLRTVQRLVKNWEQDGLVGLTQTSRADKGKHRIGEFWENFITKT
YKEGNKGSKRMTPKQVALRVEAKARELKDSKPPNYKTVLRVLAPILEKQQKAKSIRSPG
WRGTTLSVKTREGKDLSVDYSNHVWQCDHTRVDVLLVDQHGEILSRPWLTTVIDTYSRC
IMGINLGFDAPSSGVVALALRHAILPKRYGSEYKLHCEWGTYGKPEHFYTDGGKDFRSN
HLSQIGAQLGFVCHLRDRPSEGGVVERPFKTLNDQLFSTLPGYTGSNVQERPEDAEKDA
RLTLRELEQLLVRYIVDRYNQSIDARMGDQTRFERWEAGLPTVPVPIPERDLDICLMKQ
SRRTVQRGGCLQFQNLMYRGEYLAGYAGETVNLRFDPRDITTILVYRQENNQEVFLTRA
HAQGLETEQLALDEAEAASRRLRTAGKTISNQSLLQEVVDRDALVATKKSRKERQKLEQ
TVLRSAAVDESNRESLPSQIVEPDEVESTETVHSQYEDIEVWDYEQLREEYGF
CAST system, CRISPR-associated transposase
from cyanobacteria Scytonema hofmanni-
ShTnsC - WP_029636336.1 SEQ ID NO: 437
MTEAQAIAKQLGGVKPDDEWLQAEIARLKGKSIVPLQQVKTLHDWLDGKRKARKSCRVV
GESRTGKTVACDAYRYRHKPQQEAGRPPTVPVVYIRPHQKCGPKDLFKKITEYLKYRVT
KGTVSDFRDRTIEVLKGCGVEMLIIDEADRLKPETFADVRDIAEDLGIAVVLVGTDRLD
AVIKRDEQVLERFRAHLRFGKLSGEDFKNTVEMWEQMVLKLPVSSNLKSKEMLRILTSA
TEGYIGRLDEILREAAIRSLSRGLKKIDKAVLQEVAKEYK
INTEGRATE system - Vibrio cholerae TniQ, Cas8,
Cas7 ,and Cas6 (CASCADE) SEQ ID NO: 438
MFLQRPKPYSDESLESFFIRVANKNGYGDVHRFLEATKRFLQDIDHNGYQTFPTDITRI
NPYSAKNSSSARTASFLKLAQLTFNEPPELLGLAINRTNMKYSPSTSAVVRGAEVFPRS
LLRTHSIPCCPLCLRENGYASYLWHFQGYEYCHSHNVPLITTCSCGKEFDYRVSGLKGI
CCKCKEPITLTSRENGHEAACTVSNWLAGHESKPLPNLPKSYRWGLVHWWMGIKDSEFD
HFSFVQFFSNWPRSFHSIIEDEVEFNLEHAVVSTSELRLKDLLGRLFFGSIRLPERNLQ
HNIILGELLCYLENRLWQDKGLIANLKMNALEATVMLNCSLDQIASMVEQRILKPNRKS
KPNSPLDVTDYLFHFGDIFCLWLAEFQSDEFNRSFYVSRWMQTLKELIASNPDDLTTEL
KRAFRPLTPHIAIDGNELDALTILVNLTDKTDDQKDLLDRAKCKQKLRDEKWWASCINC
VNYRQSHNPKFPDIRSEGVIRTQALGELPSFLLSSSKIPPYHWSYSHDSKYVNKSAFLT
NEFCWDGEISCLGELLKDADHPLWNTLKKLGCSQKTCKAMAKQLADITLTTINVTLAPN
YLTQISLPDSDTSYISLSPVASLSMQSHFHQRLQDENRHSAITRFSRTTNMGVTAMTCG
GAFRMLKSGAKFSSPPHHRLNSKRSWLTSEHVQSLKQYQRLNKSLIPENSRIALRRKYK
IELQNMVRSWFAMQDHTLDSNILIQHLNHDLSYLGATKRFAYDPAMTKLFTELLKRELS
NSINNGEQHTNGSFLVLPNIRVCGATALSSPVTVGIPSLTAFFGFVHAFERNINRTTSS
FRVESFAICVHQLHVEKRGLTAEFVEKGDGTISAPATRDDWQCDVVFSLILNTNFAQHI
DQDTLVTSLPKRLARGSAKIAIDDFKHINSFSTLETAIESLPIEAGRWLSLYAQSNNNL
SDLLAAMTEDHQLMASCVGYHLLEEPKDKPNSLRGYKHAIAECIIGLINSITFSSETDP
NTIFWSLKNYQNYLVVQPRSINDETTDKSSLMEAASLAYDVSGQPIKSATAEALAQGNP
HQVDFCHVPYGASHIECSFSVSFSSELRQPYKCNSSKVKQTLVQLVELYETKIGWTELA
TRYLMNICNGKWLWKNTRKAYCWNIVLTPWPWNGEKVGFEDIRTNYTSRQDFKNNKNWS
AIVEMIKTAFSSTDGLAIFEVRATLHLPTNAMVRPSQVFTEKESGSKSKSKTQNSRVFQ
STTIDGERSPILGAFKTGAAIATIDDWYPEATEPLRVGRFGVHREDVTCYRHPSTGKDF
FSILQQAEHYIEVLSANKTPAQETINDMHFLMANLIKGGMFQHKGD
INTEGRATE system - Vibrio cholerae TnsA, TnsB,
and TnsC SEQ ID NO: 439
MATSLPTPSAITTSALEYAFHTPARNLTKSRGKNIHRYVSVKMSKRITVESTLECDACY
HFDFEPSIVRFCAQPIRFLYYLNGQSHSYVPDFLVQFDTNEFVLYEVKSAYAKNKPDFD
VEWEAKVKAATELGLELELVEESDIRDTVVLNNLKRMHRYASKDELNNVHNSLLKIIKY
NGAQSARCLGEQLGLKGRTVLPILCDLLSRCLLDTRLDKPLSLESRFELASYGMSETRE
ARISRAKRAFVSTPSVRKILSYMDRCRDLSDLESEPTCMMVYGASGVGKTTVIKKYLNQ
NRRESEAGGDIIPVLHIELPDNAKPVDAARELLVEMGDPLALYETDLARLTKRLTELIP
AVGVKLIIIDEFQHLVEERSNRVLTQVGNWLKMILNKTKCPIVIFGMPYSKVVLQANSQ
LHGRFSIQVELRPFSYQGGRGVFKTFLEYLDKALPFEKQAGLANESLQKKLYAFSQGNM
RSLRNLIYQASIEAIDNQHETITEEDFVFASKLTSGDKPNSWKNPFEEGVEVTEDMLRP
PPKDIGWEDYLRHSTPRVSKPGRNKNFFEMAKKGFSSFHRKAVSSQDTLESIELVSSAN
CLESVTYQDISAFPETIAVEINFRLSILRFLARKCETIVAKSIEPHRVELQQNYSRKIP
SAITIYRWWLAFRKSDYNPISLAPNIKDRGNRETKVSTVVDSIMEQAVERVISGRKVNV
SSAYKRVRRKVRQYNLTHGTKYTYPKYESVRKRVKKKTPFELLAAGKGERVAKREFRRM
GKKILTSSVLERVEIDHTVVDLFAVHEEYRIPLGRPWLTQLVDCYSKAVIGFYLGFEPP
SYVSVSLALKNAIQRKDDLISSYESIENEWLCYGIPDLLVTDNGKEFLSKAFDQACESL
LINVHQNKVETPDNKPHVERNYGTINTSLLDDLPGKSFSQYLQREGYDSVGEATLTLNE
IREIYLIWLVDIYHKKPNQRGTNCPNVAWKKGCQEWEPEEFSGSKDELDFKFAIVDYKQ
LTKVGITVYKELSYSNDRLAEYRGKKGNHKVQFKYNPECMAVIWVLDEDMNEYFTVNAI
DYEYASRVSLWQHKYNMKYQAELNSAEYDEDKEIDAEIKIEEIADRSIVKTNKIRARRR
GARHQENSARAKSISNANPASIQKHEDEIVSADNDDWDIDYV
Escherichia phage P1 (Bacteriophage P1) Cre
Recombinase (Uniprot P06956), SEQ ID NO: 440
MSNLLTVHQNLPALPVDATSDEVRKNLMDMFRDRQAFSEHTWKMLLSVCRSWAAWCKLN
NRKWFPAEPEDVRDYLLYLQARGLAVKTIQQHLGQLNMLHRRSGLPRPSDSNAVSLVMR
RIRKENVDAGERAKQALAFERTDFDQVRSLMENSDRCQDIRNLAFLGIAYNTLLRIAEI
ARIRVKDISRTDGGRMLIHIGRTKTLVSTAGVEKALSLGVTKLVERWISVSGVADDPNN
YLFCRVRKNGVAAPSATSQLSTRALEGIFEATHRLIYGAKDDSGQRYLAWSGHSARVGA
ARDMARAGVSIPEIMQAGGWTNVNIVMNYIRNLDSETGAMVRLLEDGD

What is claimed is:

1. A method for identifying a genetic modifier to alter expression of a target gene in a selected cell type, the method comprising:
providing, optionally from a database, one or more candidate regulatory motif sequences with regulatory potential in the selected cell type;
selecting a sequence of a putative regulatory region of the target gene, preferably wherein the putative regulatory region is in a promoter, enhancer, insulator, untranslated region (UTR), or intron, optionally in a non-coding region of the target gene;
comparing the sequence of the putative regulatory region to the candidate regulatory motif sequences,
identifying a candidate regulatory motif sequence that has either no identity at all as a potential insertion site or that has at least 500% identity and at least one mismatch to a portion of the sequence of the regulatory sequence as a potential substitution site;
determining sequence alterations needed to make the putative regulatory region match the candidate regulatory motif sequence; and
identifying one or more genetic modifiers capable of making the sequence alterations needed to make the putative regulatory region match the candidate regulatory motif sequence, or;
optionally wherein identifying a genetic modifier comprises using an algorithm that compares the putative regulatory region and candidate regulatory motif sequences from a database and identifies candidate regulatory motif sequences that differ from the putative regulatory region by at least one nucleotide and up to 100% as a potential insertion site, or that differ from the putative regulatory region by at least one nucleotide and up to a selected amount, optionally at least 50% identity, as a potential substitution site, determines sequence alterations needed to make the putative regulatory region match the candidate regulatory motif sequence, compares the sequence alterations to a database of modifications that could be made by a set of genetic modifiers, and identifying one or more genetic modifiers that can alter the putative regulatory region to match the candidate regulatory motif sequence, to thereby introduce a functional regulatory motif.

2. The method of claim 1, wherein the candidate regulatory sequence motif has regulatory potential to affect target gene expression at the pre-transcriptional or post-transcriptional level.

3. The method of claim 2, wherein the candidate regulatory sequence motif is a transcription factor binding sequence that can recruit endogenous transcription factors within a cell type or cell types of interest, where the sequence motif may or may not exist in the genome of the selected cell type.

4. The method of claim 2, wherein the candidate regulatory sequence motif alters spacing of endogenous transcription factor binding sites in the putative regulatory region.

5. The method of claim 2, wherein the candidate regulatory sequence motif is a response element that is activated by a receptor-ligand complex through binding of an exogenously delivered small molecule, hormone, or drug for inducible target gene activation.

6. The method of claim 2, wherein the candidate regulatory sequence motif either stabilizes or de-stabilizes target gene transcripts, where the candidate regulatory sequence motif may or may not exist in the genome of the selected cell type.

7. The method of claim 2, wherein the candidate regulatory sequence motif is a hybridization target for endogenous non-coding RNAs, where the sequence motif that may or may not exist in the genome of interest.

8. The method of claim 2, wherein the candidate regulatory sequence motif modifies the translation initiation and/or elongation efficiency for target gene transcripts, and wherein the candidate regulatory sequence motif may or may not exist in the genome of the selected cell type.

9. The method of claims 1-8, wherein the putative regulatory region has the potential to modify expression of the target gene at the pre-transcriptional or post-transcriptional level.

10. The method of claim 9, wherein the putative regulatory region is a non-coding DNA sequence within 1 Mb or more of a target gene of interest, or spatially-proximal as determined by chromosome conformation capture assays.

11. The method of claim 9, wherein the putative regulatory region is a promoter of a target gene of interest.

12. The method of claim 9, wherein the putative regulatory region comprises putative enhancer elements of a target gene of interest as defined by histone marks associated and/or chromatin accessibility features associated with functional enhancer elements; putative insulator elements of the target gene of interest as defined by histone marks associated and/or chromatin accessibility features associated with functional insulator elements; and/or putative silencer elements of the target genes of interest as defined by histone marks associated and/or chromatin accessibility features associated with functional silencer elements.

13. The method of claim 9, wherein the putative regulatory region comprises untranslated regions (UTRs) of the target gene transcripts.

14. The method of claim 9, wherein the putative regulatory regions comprise an intronic region of the target gene transcripts.

15. The method of claim 9, wherein the putative regulatory regions comprises a coding sequence of target gene transcripts.

16. The method of claims 1-15, wherein the identified genetic modifier can introduce a specific sequence motif or modification at the target genomic region.

17. The method of claim 16, wherein the genetic modifier comprises a CRISPR-Cas domain, a zinc-finger DNA binding domain, or a transcription activator-like (TAL) effector domain.

18. The method of claim 17, wherein the CRISPR-Cas domain is used with a gRNA, wherein the gRNA comprises a sequence complementary to a sequence of the target cis-regulatory element of interest.

19. The method of claim 16, wherein the genetic modifier is a programmable nuclease, a base editor, or a prime editor.

20. The method of claim 17, wherein the CRISPR-Cas prime editor further comprises a prime editing gRNA (pegRNA) and nicking sgRNA (ngRNA) wherein the pegRNA and ngRNA comprise a sequence complementary to a sequence of the target cis-regulatory element of interest.

21. A method for altering expression of a target gene in a selected cell type, the method comprising:
providing, optionally from a database, one or more candidate regulatory motif sequences with regulatory potential in the selected cell type;
selecting a sequence of a putative regulatory region of the target gene, preferably wherein the putative regulatory region is in a promoter, enhancer, insulator, untranslated region (UTR), or intron, optionally in a non-coding region of the target gene;
comparing the sequence of the putative regulatory region to the candidate regulatory motif sequences,
identifying a candidate regulatory motif sequence that has either no identity at all as a potential insertion site or that has at least 50% identity and at least one mismatch to a portion of the sequence of the regulatory sequence as a potential substitution site;
determining sequence alterations needed to make the putative regulatory region match the candidate regulatory motif sequence; and
identifying one or more genetic modifiers capable of making the sequence alterations needed to make the putative regulatory region match the candidate regulatory motif sequence, and
contacting the cell with the one or more genetic modifiers under conditions and for a time sufficient for the one or more genetic modifiers to make the putative regulatory region match the candidate regulatory motif sequence.

22. The method of claim 21, wherein identifying a genetic modifier comprises using an algorithm that compares the putative regulatory region and candidate regulatory motif sequences from a database and identifies candidate regulatory motif sequences that differ from the putative regulatory region by at least one nucleotide and up to 100% as a potential insertion site, or that differ from the putative regulatory region by at least one nucleotide and up to a selected amount, optionally at least 50% identity, as a potential substitution site, determines sequence alterations needed to make the putative regulatory region match the candidate regulatory motif sequence, compares the sequence alterations to a database of modifications that could be made by a set of genetic modifiers, and identifying one or more genetic modifiers that can alter the putative regulatory region to match the candidate regulatory motif sequence, to thereby introduce a functional regulatory motif.

23. A method for altering expression of a target gene in a selected cell, the method comprising contacting the selected cell with a genetic modifier identified using the method of claims 1-20, under conditions sufficient to increase the target gene expression in the cell.

24. A method for heterotopic activation of a target gene expression in a selected cell, the method comprising contacting the cell with a genetic modifier identified using the method of claims 1-20, under conditions sufficient to increase the target gene expression in the cell.

25. The method of claims 21-24, wherein the candidate regulatory sequence motif is introduced into the putative regulatory region as a single motif or a repetitive sequence with multiple copies of the single motif, optionally with linker sequences therebetween.

26. The method of claims 21-24, wherein the genetic modifier introduces multiplex edits to alter expression of a single target gene.

27. The method of claims 21-24, wherein the genetic modifier introduces multiplex edits in to alter expression of multiple target genes.

28. The method of any one of claims 1-27, wherein the cell is a eukaryotic cell.

29. The method of claim 28, wherein the cell is a mammalian cell.

30. The method of claim 29, wherein the cell is a human cell.

31. A method for treating or reducing risk of a condition or a disease in a subject, wherein the condition or the disease is caused, at least in part, by insufficient expression of the target gene, the method comprising administering to the subject an effective amount of a genetic modifier identified using the method of any one of claims 1-20, under conditions sufficient to increase the target gene expression in the cell, thereby treating or reducing risk of the condition or the disease in the subject.

32. The method of claim 31, wherein the condition or the disease is caused, at least in part, by insufficient expression of the target gene on an allele.

33. The method of claim 31, wherein the condition or the disease is related to haploinsufficiency.

34. The method of claim 31 or 32, wherein the condition or the disease is caused, at least in part, by a dominant-negative gene.

35. The method of claim 31 or 32, wherein the condition or the disease is caused, at least in part, by insufficient expression of a target gene that is under the control of an enhancer, wherein the enhancer controls the expression of a plurality of genes.

36. The method of any one of claims 31-35, wherein the method causes an increase in the expression of the target gene in the cell or in the cell of the subject by at least 1.1 fold as measured by mRNA expression.

37. A method for treating or reducing risk of a condition or a disease in a subject, wherein the condition or the disease is caused, at least in part, by overexpression of the target gene, the method comprising administering to the subject an effective amount of a genetic modifier identified using the method of any one of claims 1-20, under conditions sufficient to increase the target gene expression in the cell, thereby treating or reducing risk of the condition or the disease in the subject.

38. The method of any one of claims 31-37, wherein the method causes a decrease in the expression of the target gene in the cell or in the cell of the subject by at least 1.1 fold.
